(19) United States
(12) Patent Application Publication
(10) Pub. No.: US 2024/0239828 A1
(43) Pub. Date: Jul. 18, 2024

(54) MANNOSE 6-PHOSPHATE OR ASGPR RECEPTOR BINDING COMPOUNDS FOR THE DEGRADATION OF EXTRACELLULAR PROTEINS

(71) Applicant: Avilar Therapeutics, Inc., Waltham, MA (US)

(72) Inventors: Jason Allan Wiles, Madison, CT (US); Srinivasa Karra, Pembrooke, MA (US); Daniel Grau, Arlington, MA (US); Soumya Ray, Quincy, MA (US)

(73) Assignee: Avilar Therapeutics, Inc., Waltham, MA (US)

(21) Appl. No.: 18/586,086

(22) Filed: Feb. 23, 2024

Related U.S. Application Data
(63) Continuation of application No. PCT/US2022/041738, filed on Aug. 26, 2022.
(60) Provisional application No. 63/293,432, filed on Dec. 23, 2021.
(60) Provisional application No. 63/245,460, filed on Sep. 17, 2021.
(60) Provisional application No. 63/238,013, filed on Aug. 27, 2021.

(51) Int. Cl. C07H 7/04; C07H 7/02; C07H 7/06; C07H 13/00

(52) U.S. Cl. CPC C07H 7/04; C07H 7/02; C07H 7/06; C07H 13/00

(57) ABSTRACT

Compounds and compositions that have a mannose 6-phosphate receptor or ASGPR binding ligand bound to an extracellular protein binding ligand are provided for the selective degradation of a target extracellular protein in vivo to treat disorders mediated by the extracellular protein.

### CROSS-REFERENCE TO RELATED APPLICATIONS

This application is a continuation of International Patent Application No. PCT/US2022/041738, filed in the U.S. Receiving Office on Aug. 26, 2022, which claims the benefit of U.S. Provisional Application No. 63/238,013, filed Aug. 27, 2021, U.S. Provisional Application No. 63/245,460, filed Sep. 17, 2021, and U.S. Provisional Application No. 63/293,432, filed Dec. 23, 2021. The entirety of each of these applications is hereby incorporated by reference for all purposes.

### FIELD OF THE INVENTION

This invention provides compounds and compositions that have a mannose 6-phosphate receptor or ASGPR binding ligand bound to an extracellular protein binding ligand for the selective degradation of a target extracellular protein in vivo to treat disorders mediated by the extracellular protein.

### INCORPORATION BY REFERENCE

The contents of the XML file named “19121-009WO1_SequenceListing_ST26” which was created on Aug. 26, 2022 and is 543 KB in size, are hereby incorporated by reference in their entirety.

### BACKGROUND OF THE INVENTION

Historically, therapeutic strategies for the inhibition of proteins employed small molecule inhibitors which bound in an enzymatic pocket or at an allosteric position. Those proteins which were not enzymes were difficult to control, and some were considered “not druggable.”

Intracellular protein degradation is a natural and highly regulated, essential process that maintains cellular homeostasis. The selective identification and removal of damaged, misfolded, or excess proteins within the cell is achieved via the ubiquitin-proteasome pathway (UPP). The UPP is central to the regulation of almost all intracellular processes. A number of companies and institutions have designed intracellular protein degrading molecules that take advantage of this natural process to degrade disease-mediating proteins intracellularly by linking a ligand to the protein to be degraded to a protein in the UPP. Examples are found in U.S. 2014/0356322 assigned to Yale University, GlaxoSmithKline, and Cambridge Enterprise Limited University of Cambridge; Buckley et al. (J. Am. Chem. Soc. 2012, 134, 4465-4468) titled “Targeting the Von Hippel-Lindau E3 Ubiquitin Ligase Using Small Molecules to Disrupt the Vhl/Hif-1alpha Interaction”; WO 2015/160845 assigned to Arvinas Inc. titled “Imide Based Modulators of Proteolysis and Associated Methods of Use”; Lu et al. (Chem. Biol. 2015, 22, 755-763) titled “Hijacking the E3 Ubiquitin Ligase Cereblon to Efficiently Target Brd4”; Bondeson et al. (Nat. Chem. Biol. 2015, 11, 611-617) titled “Catalytic in Vivo Protein Knockdown by Small-Molecule Protacs”; Gustafson et al. (Angewandte Chemie, International Edition in English 2015, 54, 9659-9662) titled “Small-Molecule-Mediated Degradation of the Androgen Receptor through Hydrophobic Tagging”; Lai et al. (Angewandte Chemie, International Edition in English 2016, 55, 807-810) titled “Modular Protac Design for the Degradation of Oncogenic Bcr-Abl”; Toure et al. (Angew. Chem. Int. Ed. 2016, 55, 1966-1973) titled “Small-Molecule Protacs: New Approaches to Protein Degradation”; Winter et al. (Science 2015, 348, 1376-1381) titled “Drug Development. Phthalimide Conjugation as a Strategy for in Vivo Targeted Protein Degradation”; U.S. 2016/0058872 assigned to Arvinas, Inc. titled “Imide Based Modulators of Proteolysis and Associated Methods of Use” and U.S. 2016/0045607 assigned to Arvinas Inc. titled “Estrogen-related Receptor Alpha Based PROTAC Compounds and Associated Methods of Use”.

The highjacking of the UPP intracellular process to degrade difficult or undruggable proteins, however, is not available to degrade extracellular proteins. Nonlimiting examples of extracellular proteins that can play a strong role in creating or exacerbating serious diseases include immunoglobulins and cytokines, including IgA, IgG, IgD, IgE, and IgM.

The human body includes several cell surface receptors that function to regulate circulating extracellular protein concentration by transporting their extracellular protein target into the cell for degradation. Mannose 6-phosphate receptors (M6PR) and the asialoglycoprotein receptor (ASGPR) traffic proteins to the lysosome for degradation. There are two mannose 6-phosphate receptors, one of which is dependent on cation presence for efficient lysosomal activity (cation dependent mannose 6-phosphate receptor, also known as CD-M6PR and 46 kDa mannose 6-phosphate receptor) and another that does not depend on cation presence for efficient lysosomal activity (cation independent mannose 6-phosphate receptor, also known as CI-M6PR, IGF2 receptor, and CD222). The asialoglycoprotein receptor (ASGPR) is a Ca²⁺-dependent lectin that is primarily expressed in parenchymal hepatocyte cells. The main role of ASGPR is to help regulate serum glycoprotein levels by mediating endocytosis of desialylated glycoproteins.

Preliminary research has been reported on the use of cell surface receptors for the targeted degradation of extracellular proteins. For example, WO2021/142377 assigned to Lycia Therapeutics describes compounds with ASGPR receptor ligands or mannose 6-phosphate receptor ligands for the degradation of extracellular proteins. WO2021/156792 assigned to Novartis AG describes additional compounds with ASGPR receptor ligands or mannose 6-phosphate and describes their use for the degradation of FHR3 and PCSK9.

The Board of Trustees of the Leland Stanford Junior University has filed a PCT application, WO2020/132100, which describes the use of compounds that bind a lysosomal targeting molecule such as the mannose 6-phophate receptor or ASGPR to degrade a cell surface molecule or extracellular molecule. Compounds related to the WO2020/132100 disclosure are described in an article by Banik et al. (“Lysosome-Targeting Chimaeras for Degradation of Extracellular Proteins” Nature, 2020, 584, 291). Additional research from Bertozzi, et al. was published in an article titled “Lysosome Targeting Chimeras (LYTACs) That Engage a Liver-Specific Asialoglycoprotein Receptor for Targeted Protein Degradation,” Nature 17, 937-946, (2021).

Yale University has filed applications including WO 2019/199621, WO 2019/199634, WO 2021/072269, WO 2021/072246, WO 2022/178428, and WO 2022/178425 which describe the use of certain cellular receptor targeting ligands covalently bound to a circulating protein binding moiety.

U.S. Pat. Nos. 9,340,553; 9,617,293; 10,039,778; 10,376,531, and 10,813,942 assigned to Pfizer Inc. describe certain bicyclic, bridged ketal derivatives of N-acetylgalactosamine (GalNAc) as targeting agents for the ASGPR receptor that in one embodiment are bound to a linker and/or a therapeutic agent.

Avilar Therapeutics Inc. describes new ASGPR binding ligands with various C2 substituents in WO2021/155317.

While some progress has been made in the area of targeted degradation of disease-mediating extracellular proteins, much is left to be accomplished. There remains an unmet need for additional chemical compounds and approaches to treat medical disorders mediated by extracellular proteins.

### SUMMARY OF THE INVENTION

Novel compounds and their pharmaceutically acceptable salts and compositions thereof that degrade disease-mediating extracellular proteins, as well as starting materials and intermediates for such compounds and their methods of use and processes of manufacture are provided. The invention provides new extracellular protein degraders that include (i) a ligand for the mannose 6-phosphate receptor; (ii) a ligand for the ASGPR receptor; or (iii) both. In certain aspects the compounds of the present invention include new improved ligands related to mannose 6-phosphate, galactose, and talose which bind their target membrane bound protein with improved activity. The structures of mannose 6-phosphate, galactose, and talose are:

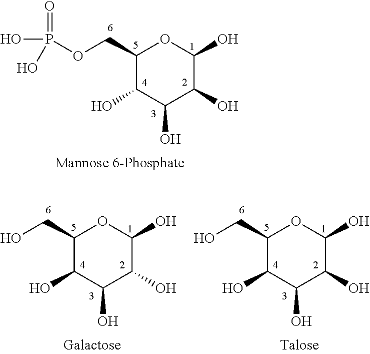 [SMILES: O=P(O)(O)OC[C@H]1O[C@@H](O)[C@@H](O)[C@@H](O)[C@@H]1O ; OC[C@H]1O[C@@H](O)[C@H](O)[C@@H](O)[C@H]1O ; OC[C@H]1O[C@@H](O)[C@@H](O)[C@@H](O)[C@H]1O]

The extracellular protein degrading compounds described herein can be used to degrade a selected extracellular protein by attaching a ligand for the extracellular protein to a selected cell surface receptor ligand, for example a mannose 6-phosphate receptor ligand (for example which can be cation independent or cation dependent) or ASGPR receptor ligand. Extracellular proteins that can be targeted according to the present invention include but are not limited to immunoglobulins such as IgA, IgG, IgD, IgE, and IgM and cytokines such as interferons, interleukins, chemokines, lymphokines, MIP, growth factors, hormones, complement factors, neurotransmitters, capsids, soluble receptors, enzymes, and tumor necrosis factors. In certain embodiments, the extracellular protein is selected from IgA, IgG, IgE, TNF (α or β), IL-1b, IL-2, IFN-γ, IL-6, VGEF, TGF-b1, PCSK-9, Factor B, Factor D, Factor H and CC5. In other nonlimiting embodiments, a compound of the present invention crosses the blood brain barrier and can be used in the treatment of a CNS related disorder such as Alzheimer's disease.

In certain aspects, an extracellular protein degrader of Formula I, Formula II, or Formula III is provided that includes a new Mannose 6-Phosphate Ligand:

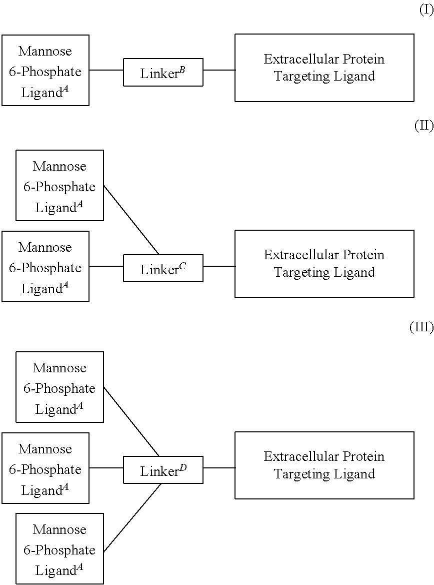

or a pharmaceutically acceptable salt thereof, wherein the variables are as defined herein.

In certain aspects, an extracellular protein degrader of Formula IV, Formula V, or Formula VI is provided that includes a new ASGPR Ligand:

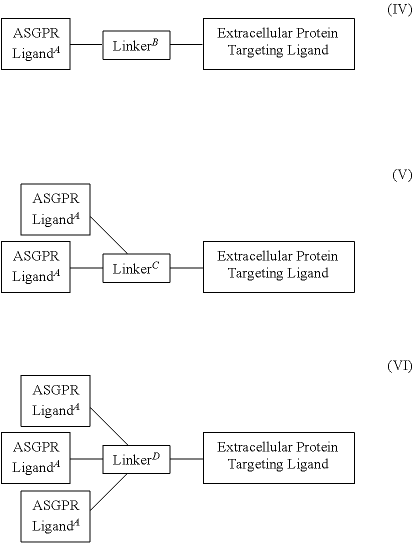

or a pharmaceutically acceptable salt thereof, wherein the variables are as defined herein.

In other aspects an extracellular protein degrader of Formula VII, Formula VIII, or Formula IX is provided that includes a mixture of Mannose 6-Phosphate Ligands and ASGPR Ligands:

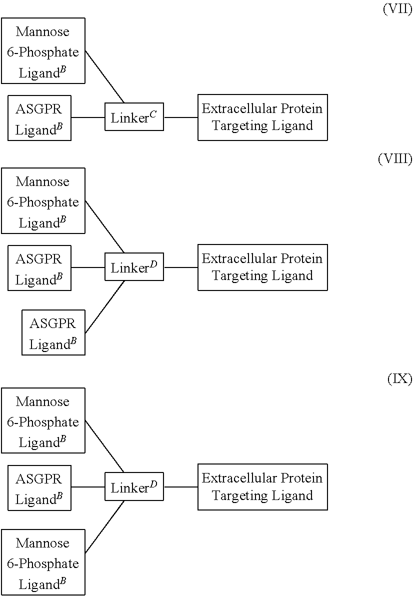

wherein:

Mannose 6-Phosphate Ligand^{A} is selected from:

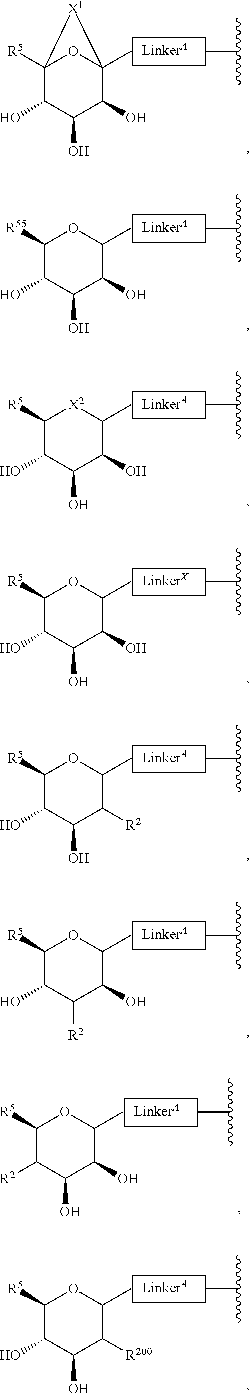

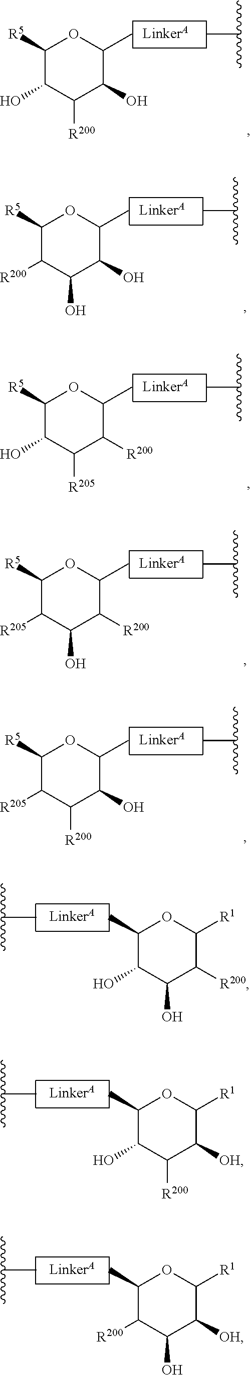

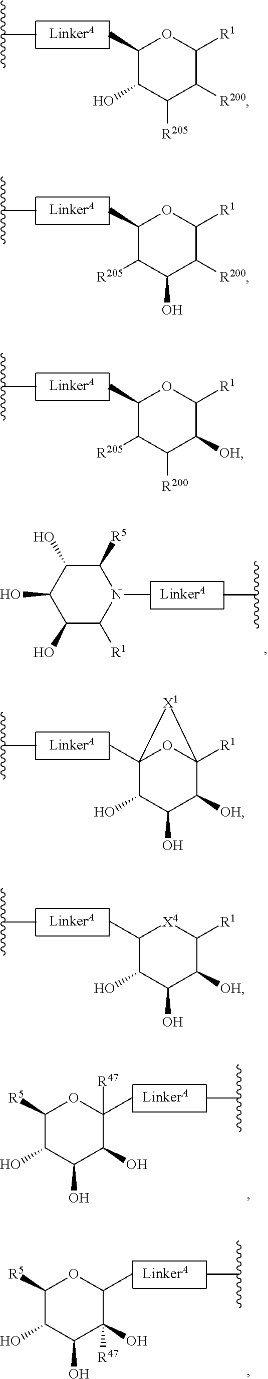

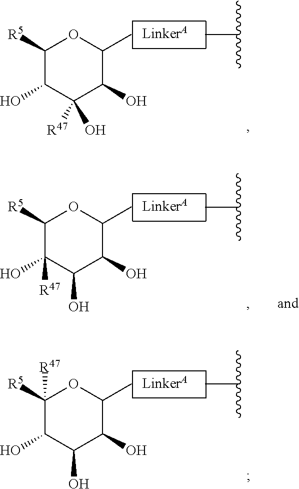

Mannose 6-Phosphate Ligand^{B} is selected from:
(i) Mannose 6-Phosphate Ligand^{A}; and

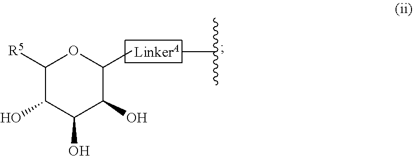

ASGPR Ligand^{A} is selected from:

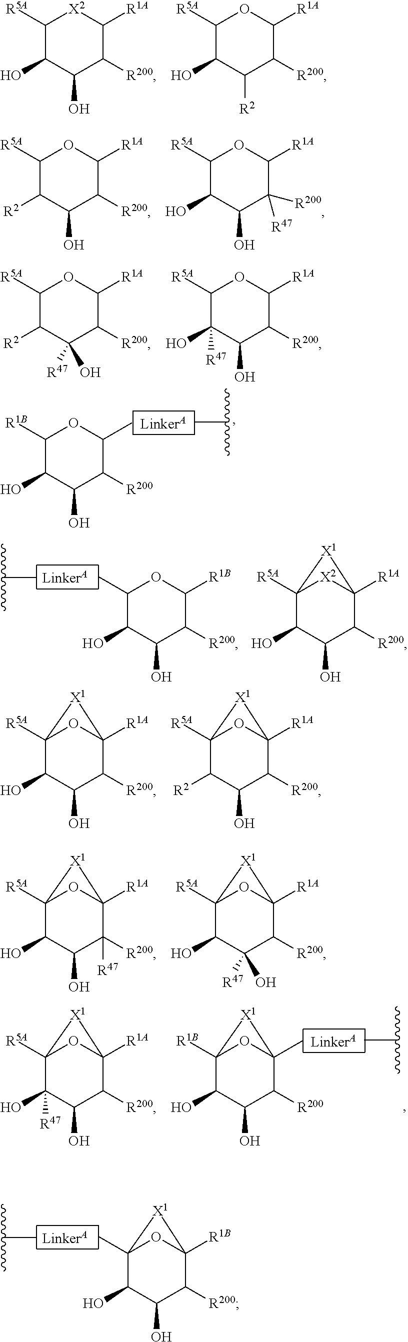

ASGPR Ligand^{B} is selected from:
(i) ASGPR Ligand^{A};

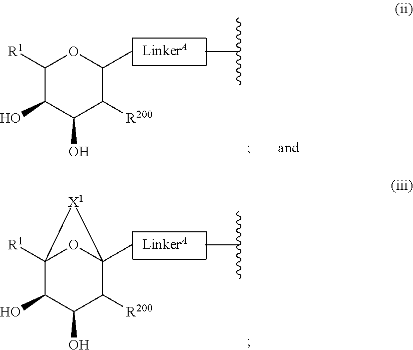

X¹ is 1 to 5 contiguous atoms independently selected from O, S, S(O), S(O)₂, N(R⁶), and C(R⁴)(R⁴), wherein if X¹ is 1 atom then X¹ is O, S, S(O), S(O)₂, N(R⁶), or C(R⁴)(R⁴), if X¹ is 2 atoms then no more than 1 atom of X¹ is O, S, or N(R⁶), if X¹ is 3, 4, or 5 atoms then no more than 2 atoms of X¹ are O, S, or N(R⁶);
X² is NR⁸, S, NC(O)R³, CR⁴R⁴, or CR⁴R⁸;
X³ is NR⁸, S, NC(O)R³, CR⁴R⁴, or CR⁴R⁸;
X⁴ is NR⁸, S, NC(O)R³, CR⁴R⁴, or CR⁴R⁸;
R² is selected from F, SH, OR⁴³, and NHR⁸;
R²⁰⁰ and R²⁰⁵ are independently selected from hydrogen, halogen, —SH, —O-alkyl, —NR⁸—C(O)—R³, —NR⁶-alkyl, aryl, heterocycle, heteroaryl, NR⁸—S(O)₂—R³, —NR⁶-heteroalkyl, —NR⁸—S(O)—R³, —NR⁸—C(S)—R³, —NR⁸—S(O)(NR⁶)—R³, —N═S(O)(R³)₂, —NR⁸C(O)NR⁹S(O)₂R³, —NR⁸—S(O)₂—R¹⁰, —NR⁸—C(NR⁶)—R³, R¹⁰, alkyl-C(O)—R³, —C(O)—R³, alkyl, haloalkyl, —OC(O)R³, and —NR⁸—C(O)R¹⁰ each of which is optionally substituted with 1, 2, 3, or 4 substituents;
R¹⁰ is selected from alkenyl, allyl, alkynyl, —NR⁶-alkenyl, —O-alkenyl, —NR⁶-alkynyl, —NR⁶-heteroaryl, —NR⁶-aryl, —O-heteroaryl, —O-aryl, —O-alkynyl, aryl, alkyl-NR⁸—C(O)—R³, alkyl-aryl, alkyl-heteroaryl with 1, 2, or 4 heteroatoms, alkyl-cyano, alkyl-OR⁶, alkyl-NR⁶R⁸, NR⁸—NR⁶—C(O)R³, NR⁸—S(O)₂—R³, each of which R¹⁰ is optionally substituted with 1, 2, 3, or 4 substituents;
R¹ is selected from hydrogen, heteroalkyl, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-P(O)(R³)₂, C₀-C₆alkyl-OP(O)(R³)₂, C₀-C₆alkyl-NR⁸P(O)(R³)₂, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, 3, or 4 substituents;
R^{1A} and R^{5A} are independently selected from

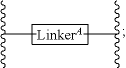

and R¹, wherein 1 of R^{1A} and R^{5A} is

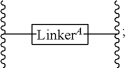

R^{1B} is selected from 4-membered heterocycle, 7-membered heterocycle, haloalkyl with 4, 5, or 6 halogen atoms, spiroheterocycle, and spirocycloalkyl;

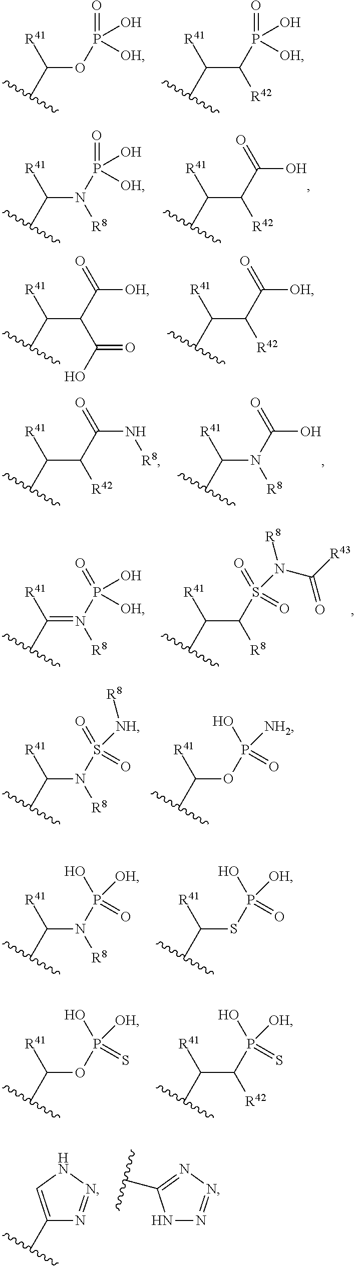

heteroaryl, heterocycle, and R⁵⁵;
R⁵⁵ is selected from

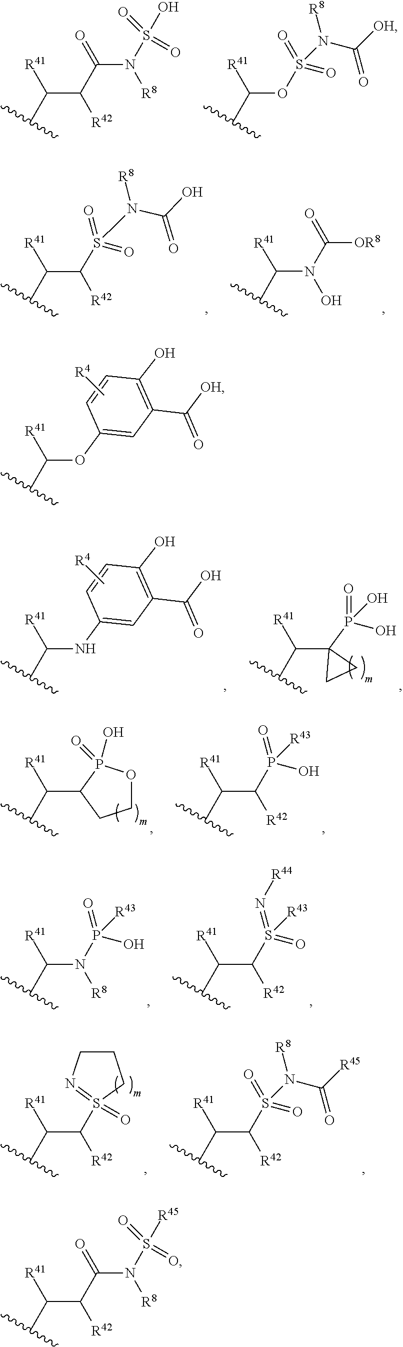

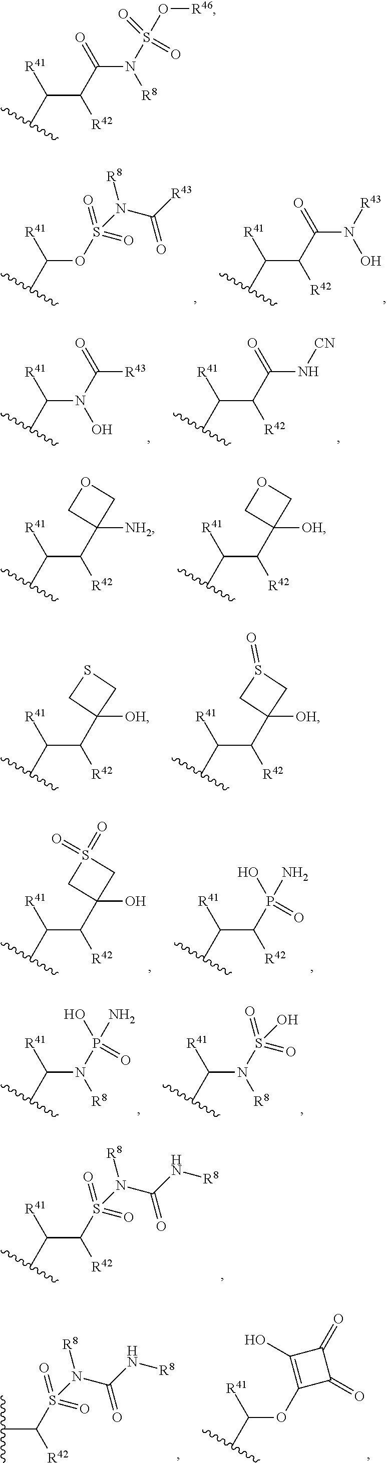

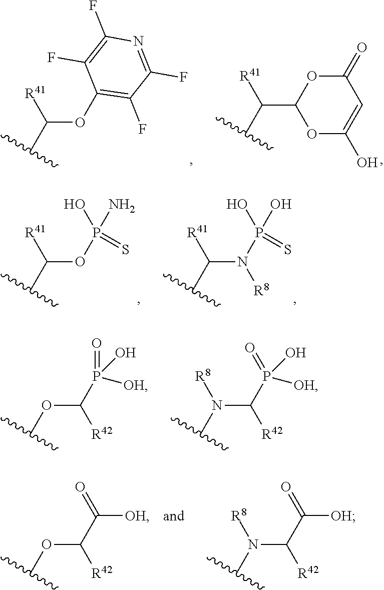

m is 1, 2, or 3;
R⁴¹ and R⁴² are independently selected from hydrogen, heteroalkyl, alkyl, arylalkyl, heteroarylalkyl, alkenyl, alkynyl, aryl, heteroaryl, and heterocycle;
or R⁴¹ and R⁴² are independently selected from hydrogen, halogen, heteroalkyl, alkyl, arylalkyl, heteroarylalkyl, alkenyl, alkynyl, aryl, heteroaryl, and heterocycle;
R⁴³ is selected from alkyl, alkenyl, alkynyl, aryl, -alkyl-aryl, heteroaryl, -alkyl-heteroaryl, heterocycle, and -alkyl-heterocycle, each of which is optionally substituted with 1, 2, 3, or 4 substituents;
R⁴⁴ is selected from hydrogen, alkyl, cyano, alkenyl, alkynyl, aryl, heteroaryl, and heterocycle each of which except hydrogen and cyano is optionally substituted with 1, 2, 3, or 4 substituents;
R⁴⁵ is selected from cycloalkyl, aryl, heteroaryl, and heterocycle each of which is optionally substituted with 1, 2, 3, or 4 substituents;
R⁴⁶ is selected from hydrogen, alkyl, alkenyl, alkynyl, aryl, heteroaryl, and heterocycle each of which except hydrogen is optionally substituted with 1, 2, 3, or 4 substituents;
R⁴⁷ is selected from C₁-C₃ alkyl and C₁-C₃ haloalkyl;
R³ at each occurrence is independently selected from hydrogen, alkyl, heteroalkyl, haloalkyl, arylalkyl, heteroarylalkyl, alkenyl, alkynyl, aryl, heteroaryl, heterocycle, —OR⁸, and —NR⁸R⁹;
R⁴ is independently selected at each occurrence from hydrogen, halogen, heteroalkyl, alkyl, haloalkyl, arylalkyl, heteroarylalkyl, alkenyl, alkynyl, aryl, heteroaryl, heterocycle, —OR⁸, —NR⁶R⁷, C(O)R³, S(O)R³, C(S)R³, and S(O)₂R³;
R⁶ and R⁷ are independently selected at each occurrence from hydrogen, heteroalkyl, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, heteroalkyl, alkyl, arylalkyl, heteroarylalkyl, alkenyl, alkynyl, aryl, heteroaryl, and heterocycle;
each Linker^{X} is selected from Linker^{Y} and Linker^{Z};
each Linker^{Y} is selected from:

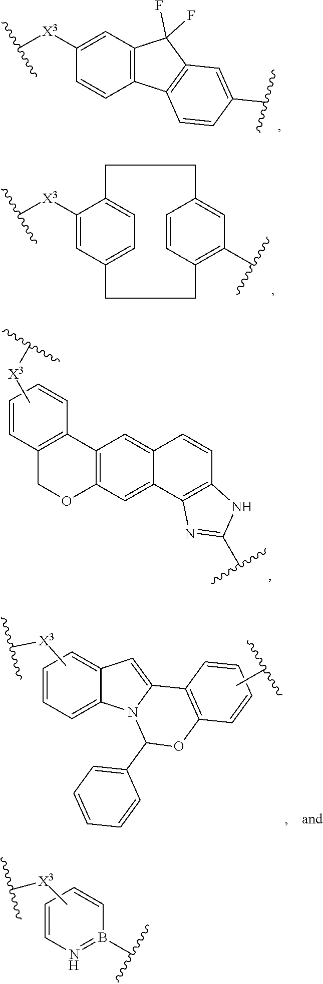

each of which is optionally substituted with 1, 2, 3, or 4 substituents;
each Linker^{Z} is selected from bivalent heteroaryl, aryl, heterocycle, cycloalkyl, —NR⁸C(O)-aryl, and —NR⁸C(O)-heteroaryl each of which is optionally substituted with 1, 2, 3, or 4 substituents;
each Linker^{A} is a bond or a moiety that covalently links the sugar (e.g. the C1 or C5 position) to Linker^{B};
Linker^{B} is a bond or a moiety that covalently links Linker^{A} to an Extracellular Protein Targeting Ligand;
Linker^{C} is a moiety that covalently links Linker^{A} to an Extracellular Protein Targeting Ligand;
Linker^{D} is a moiety that covalently links Linker^{A} to an Extracellular Protein Targeting Ligand;
Extracellular Protein Targeting Ligand is a chemical moiety that binds to the targeted disease-mediating extracellular protein; and
when a compounds is “optionally substituted” it may be substituted as allowed by valence with one or more groups selected from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, heteroalkyl, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, —SF₅,

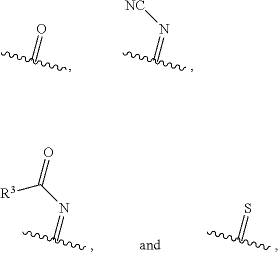

wherein the optional substituent is selected such that a stable compound results.

In certain embodiments the Mannose 6-Phosphate Ligand binds the cation independent mannose 6-phosphate receptor (which is also known as CI-MPR, IGF2 receptor, or CD222). In other embodiments the Mannose 6-Phosphate Ligand binds the cation dependent mannose 6-phosphate receptor (which is also known as CD-MPR and the 46 kDa mannose 6-phosphate receptor). In certain embodiments the Mannose 6-Phosphate Ligand binds to both the cation independent mannose 6-phosphate receptor and the cation dependent mannose 6-phosphate receptor.

In an embodiment of the invention, the Extracellular Protein Targeting Ligand is a small organic molecule (i.e., a non-biologic) that adequately binds to the protein in such a manner that it is able to transport it to the ASGPR or mannose 6-phosphate receptor, the residue of a pharmaceutically active compound that binds to the target extracellular protein (for example but not limited to a compound of the sort that would be reviewed as a drug by CDER of the FDA, or an approved or clinical stage drug) or a peptide, protein or biologic or a binding fragment thereof that adequately binds to the protein in such a manner that it is able to transport it to the ASGPR or mannose 6-phosphate receptor. A plethora of illustrative nonlimiting examples of Extracellular Protein Targeting Ligands is provided in FIG. 1.

In certain embodiments the extracellular protein is a membrane bound protein.

The extracellular protein degraders of the present invention can be administered in any manner that allows the degrader to bind to the Extracellular Protein, typically in the blood stream, and carry it to a mannose 6-phosphate receptor bearing or ASGPR-bearing cell for endocytosis and degradation. As such, examples of methods to deliver the degraders of the present invention include, but are not limited to, oral, intravenous, buccal, sublingual, subcutaneous and transnasal.

In other aspects of the present invention new mannose 6-phosphate receptor ligands are provided of Formula XX:

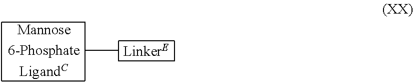

wherein:
Mannose 6-Phosphate Ligand^{C} is selected from:

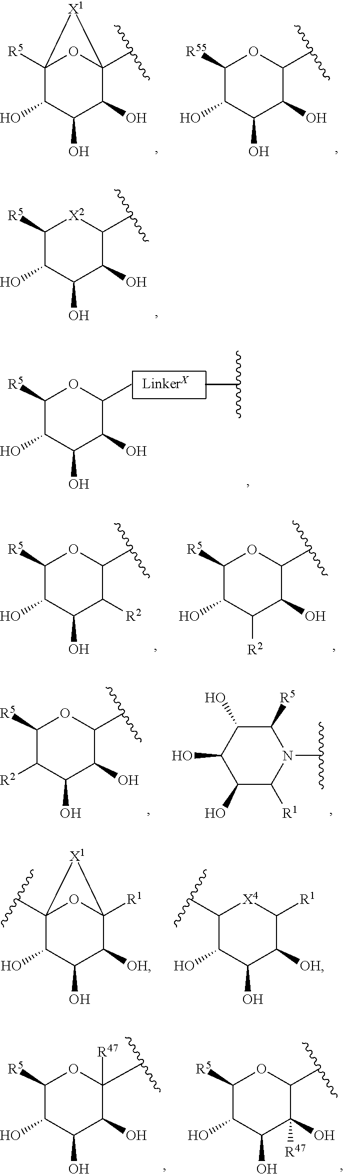

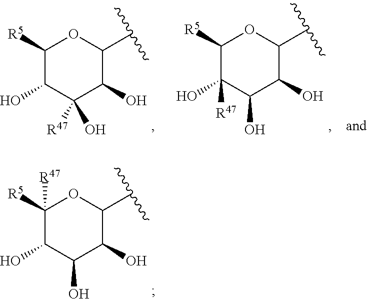

or Mannose 6-Phosphate Ligand^{C} is selected from:

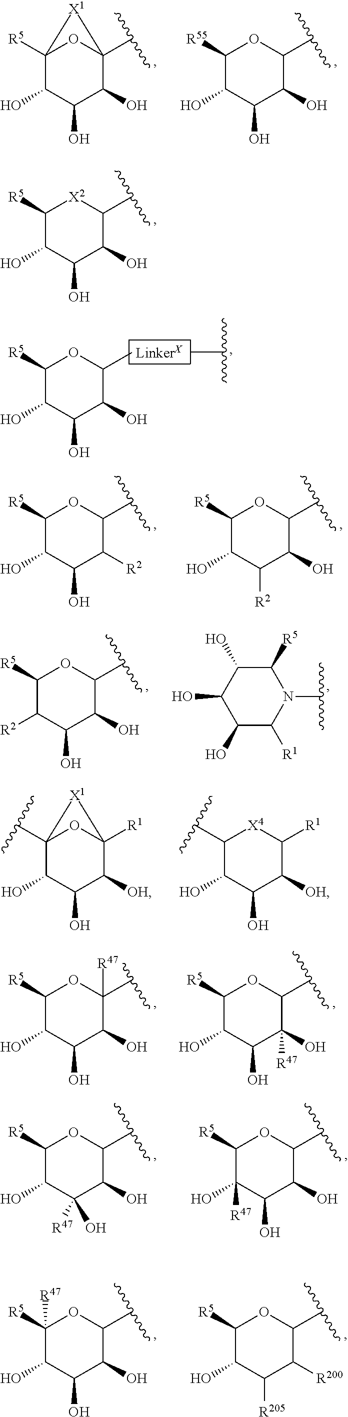

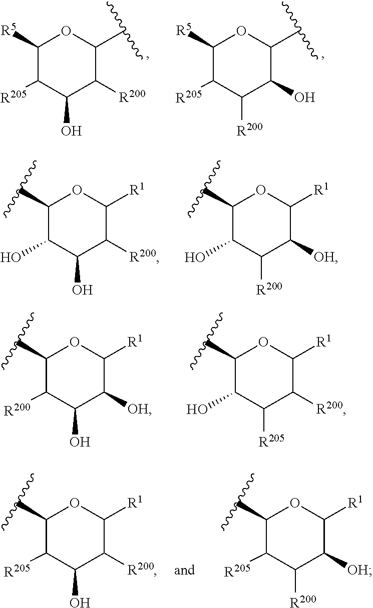

Linker^{E} is selected from

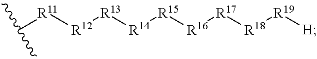

R¹¹, R¹², R¹³, R¹⁴, R¹⁵, R¹⁶, R¹⁷, R¹⁸, and R¹⁹ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —C(O)N(OH)—, —N(OH)C(O)—, —O—, —S—, —NR⁶—, —C(R²¹R²¹)—, —S(O)(═N—R⁴⁴)—, —S(O)(═NR), —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, aryl, heteroaryl, and heterocycle;
and all other variables are as defined herein.

The compounds of Formula XX can be used as intermediates in the manufacture of compounds of the present invention, probe molecules for assessing the effect of binding the cation independent or cation dependent mannose 6-phosphate receptor, therapeutics for the treatment of diseases mediated by mannose 6-phosphate, or for other uses.

In other aspects of the present invention new ASGPR receptor ligands are provided of Formula XXI:

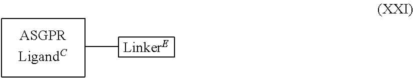

wherein
ASGPR Ligand^{C} is selected from:

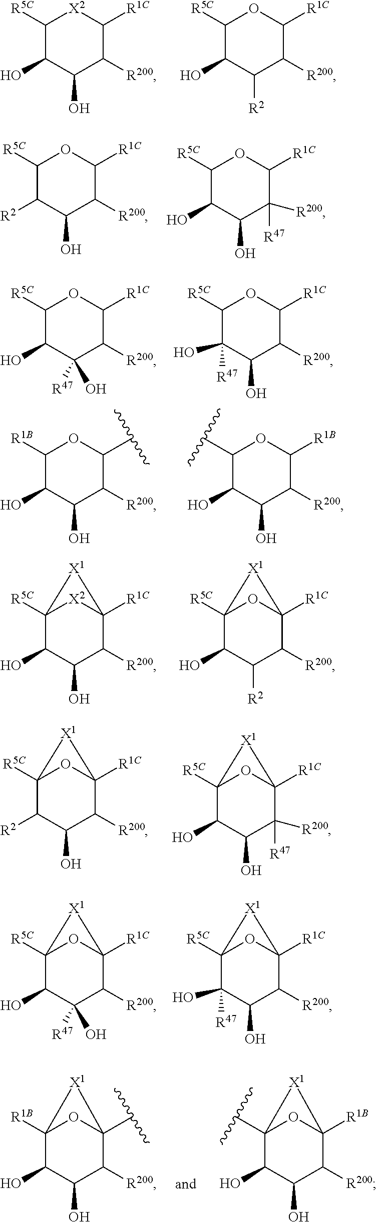

R^{1C} and R^{5C} are selected from bond to Linker^{E} and R¹, wherein one of R^{1C} and R^{5C} is bond to Linker^{E};
and wherein all other variables are as defined herein.

The compounds of Formula XXI can be used as intermediates in the manufacture of compounds of the present invention, probe molecules for assessing the effect of binding the ASGPR receptor, therapeutics for the treatment of diseases mediated by ASGPR, or for other uses.

In certain aspects one or more of the mannose 6-phosphate receptor, endocytosis associated receptor, or ASGPR ligands is an oligomer. For example, in certain embodiments the compound of the present invention is selected from:

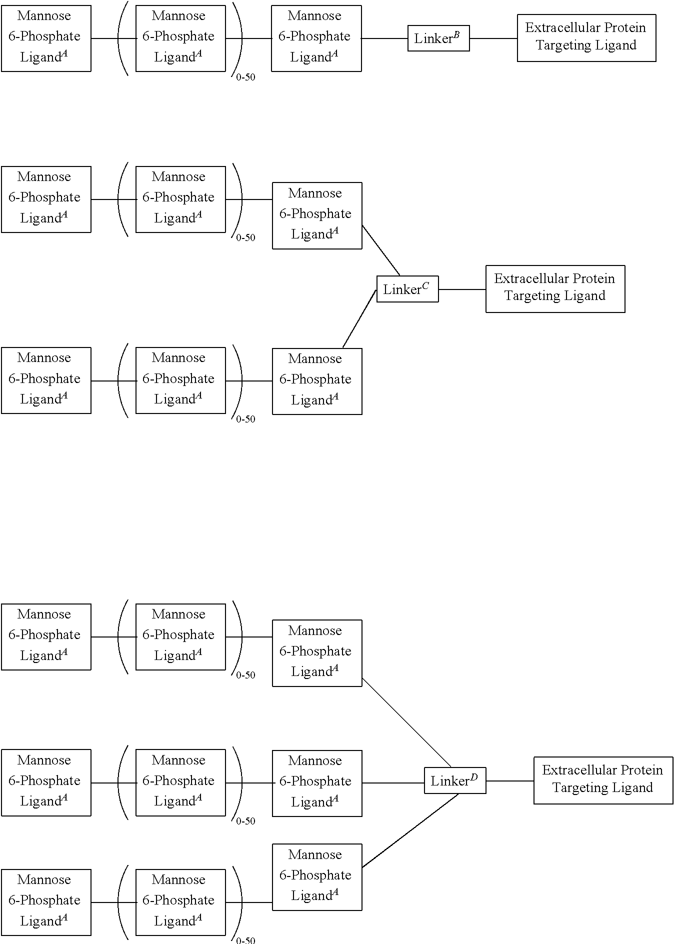

When presented as an oligomer the mannose 6-phosphate receptor ligand may only have the phosphate group or analog thereof on the terminal position or may have the phosphate group or analog thereof in between each sugar moiety. For example, oligomers of Formula I include both.

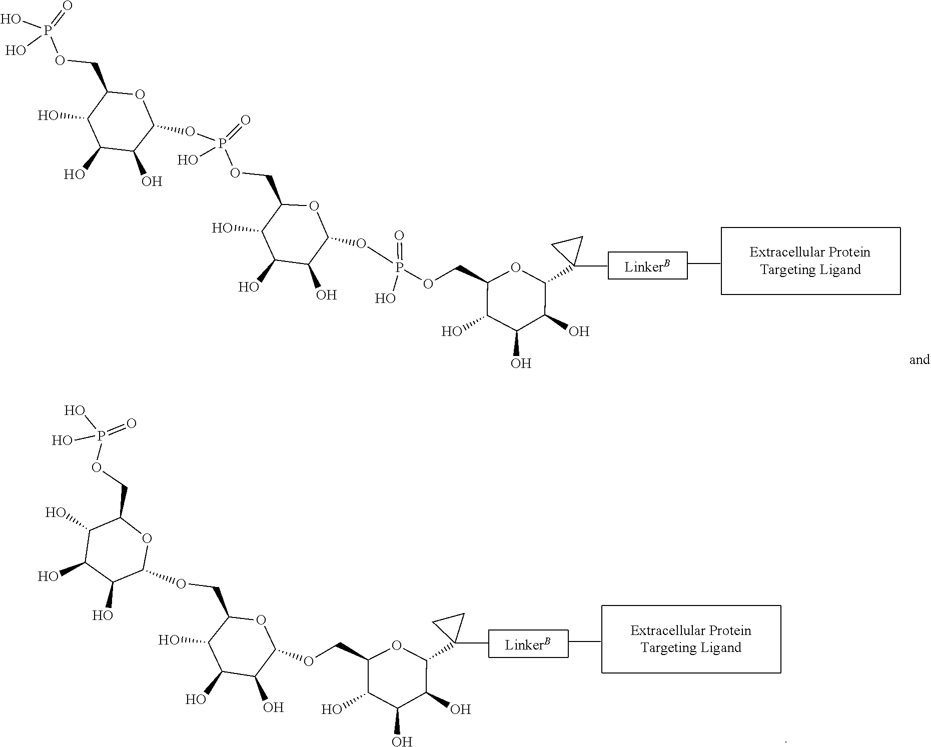

### BRIEF DESCRIPTION OF FIGURES

FIG. 1A provides a non-limiting list of Extracellular Protein Targeting Ligands that target Immunoglobulin A (IgA).

FIG. 1B provides a non-limiting list of Extracellular Protein Targeting Ligands that target Immunoglobulin G (IgG).

FIG. 1C-1G provides a non-limiting list of Extracellular Protein Targeting Ligands that target Immunoglobulin E (IgE).

FIG. 1H-1M provides a non-limiting list of Extracellular Protein Targeting Ligands that target Tumor Necrosis Factor alpha (TNF-α).

FIG. 1N provides a non-limiting list of Extracellular Protein Targeting Ligands that target Interleukin-1 (IL-1).

FIG. 1O-1S provides a non-limiting list of Extracellular Protein Targeting Ligands that target Interleukin-2 (IL-2).

FIG. 1T-1W provides a non-limiting list of Extracellular Protein Targeting Ligands that target Interleukin-6 (IL-6).

FIG. 1X-IAA provides a non-limiting list of Extracellular Protein Targeting Ligands that target Interferon gamma (IFN-γ).

FIG. 1BB-1KK provides a non-limiting list of Extracellular Protein Targeting Ligands that target Vascular endothelial growth factor (VEGF).

FIG. 1LL provides a non-limiting list of Extracellular Protein Targeting Ligands that target Transforming growth factor beta (TGF-β1).

FIG. 1MM-1PP provides a non-limiting list of Extracellular Protein Targeting Ligands that target proprotein convertase subtilisin kexin 9 (PCSK-9).

FIG. 1QQ-1SS provides a non-limiting list of Extracellular Protein Targeting Ligands that target Carboxypeptidase B2 (CPB2).

FIG. 1TT-1UU provides a non-limiting list of Extracellular Protein Targeting Ligands that target Cholinesterase (ChE).

FIG. 1VV-1WW provides a non-limiting list of Extracellular Protein Targeting Ligands that target C-C Motif Chemokine Ligand 2 (CCL2).

FIG. 1XX-1BBB provides a non-limiting list of Extracellular Protein Targeting Ligands that target coagulation factor VII (Factor VII).

FIG. 1CCC-1FFF provides a non-limiting list of Extracellular Protein Targeting Ligands that target coagulation factor IX (Factor IX).

FIG. 1GGG provides a non-limiting list of Extracellular Protein Targeting Ligands that target CD40 Ligand (CD40L).

FIG. 1HHH-1JJJ provides a non-limiting list of Extracellular Protein Targeting Ligands that target coagulation factor Xa (Factor Xa).

FIG. 1KKK-1MMM provides a non-limiting list of Extracellular Protein Targeting Ligands that target coagulation factor XI (Factor XI).

FIGS. 1NNN and 1OOO provides a non-limiting list of Extracellular Protein Targeting Ligands that target coagulation factor XII (Factor XII).

FIGS. 1PPP and 1QQQ provides a non-limiting list of Extracellular Protein Targeting Ligands that target coagulation factor XIII (Factor XIII).

FIG. 1RRR-1UUU provides a non-limiting list of Extracellular Protein Targeting Ligands that target fibroblast growth factor 1 (FGF1).

FIG. 1VVV-1XXX provides a non-limiting list of Extracellular Protein Targeting Ligands that target fibroblast growth factor 2 (FGF2).

FIGS. 1YYY and 1ZZZ provides a non-limiting list of Extracellular Protein Targeting Ligands that target fibronectin (FN1).

FIGS. 1AAAA and 1BBBB provides a non-limiting list of Extracellular Protein Targeting Ligands that target Interleukin-5 (TL-5).

FIG. 1CCCC provides a non-limiting list of Extracellular Protein Targeting Ligands that target Interleukin-8 (IL-8).

FIGS. 1DDDD and 1EEEE provides a non-limiting list of Extracellular Protein Targeting Ligands that target Interleukin-10 (IL-10).

FIGS. 1FFFF and 1GGGG provides a non-limiting list of Extracellular Protein Targeting Ligands that target Interleukin-21 (IL-21).

FIGS. 1HHHH and 1IIII provides a non-limiting list of Extracellular Protein Targeting Ligands that target Interleukin-22 (IL-22).

FIG. 1JJJJ-1NNNN provides a non-limiting list of Extracellular Protein Targeting Ligands that target Kallikrein 1.

FIG. 1OOOO provides a non-limiting list of Extracellular Protein Targeting Ligands that target lipoprotein lipase (LPL).

FIGS. 1PPPP and 1QQQQ provides a non-limiting list of Extracellular Protein Targeting Ligands that target matrix metalloproteinase-1 (MMP1).

FIG. 1RRRR-1DDDDD provides a non-limiting list of Extracellular Protein Targeting Ligands that target Macrophage migration inhibitory factor (MIF), also known as glycosylation-inhibiting factor (GIF), L-dopachrome isomerase, or phenylpyruvate tautomerase.

FIG. 1EEEEE-1GGGGG provides a non-limiting list of Extracellular Protein Targeting Ligands that target neutrophil elastase (NE).

FIGS. 1HHHHH and 1IIIII provides a non-limiting list of Extracellular Protein Targeting Ligands that target Prothrombin.

FIG. 1JJJJJ-1NNNNN provides a non-limiting list of Extracellular Protein Targeting Ligands that target Plasma kallikrein (KLKB1).

FIG. 1OOOOO-1SSSSS provides a non-limiting list of Extracellular Protein Targeting Ligands that target plasminogen (PLG).

FIG. 1TTTTT-1XXXXX provides a non-limiting list of Extracellular Protein Targeting Ligands that target Plasminogen activator inhibitor-1 (PAI-1), endothelial plasminogen activator inhibitor or serpin E1.

FIG. 1YYYYY-1AAAAAA provides a non-limiting list of Extracellular Protein Targeting Ligands that target phospholipases A2, for example type 1B or group 1B (PLA2, PA21B, PLA2G1B, PLA2-IB).

FIG. 1BBBBBB-1DDDDDD provides a non-limiting list of Extracellular Protein Targeting Ligands that target phospholipases A2, for example type IIA or group IIA (PLA2, PLA2A, PA2IIA, PLA2G2A, PLA2-IIA).

FIG. 1EEEEEE-1NNNNNN provides a non-limiting list of Extracellular Protein Targeting Ligands that target placental growth factor (PGF).

FIG. 1OOOOOO-IQQQQQQ provides a non-limiting list of Extracellular Protein Targeting Ligands that target plasminogen activator, tissue type (tPA, PLAT).

FIG. 1RRRRRR provides a non-limiting list of Extracellular Protein Targeting Ligands that target Transforming growth factor beta 2 (TGF-β2, TGFB2).

FIG. 1SSSSSS provides a non-limiting list of Extracellular Protein Targeting Ligands that target thrombospondin 1 (TSP1, TSP-1, THBS1).

FIG. 1TTTTTT-1XXXXXX provides a non-limiting list of Extracellular Protein Targeting Ligands that target Urokinase or Urokinase-type plasminogen activator (UPA, uPA).

FIG. 2 provides a non-limiting list of exemplary Extracellular Protein Targeting Ligands that target complement factor B.

FIGS. 3A and 3B provides a non-limiting list of exemplary Extracellular Protein Targeting Ligands that target complement factor D.

FIG. 4 provides a non-limiting list of exemplary Extracellular Protein Targeting Ligands that target complement factor H.

FIG. 5 provides a non-limiting list of exemplary Extracellular Protein Targeting Ligands that target complement component 5.

FIG. 6 provides a non-limiting list of exemplary Extracellular Protein Targeting Ligands that target TNF-alpha.

FIG. 7 provides a non-limiting list of exemplary Extracellular Protein Targeting Ligands that target factor XI.

FIG. 8 provides a non-limiting list of exemplary formulas of the present invention.

### DETAILED DESCRIPTION OF THE INVENTION

Novel compounds and their pharmaceutically acceptable salts and compositions thereof that degrade disease-mediating extracellular proteins, as well as starting materials and intermediates for such compounds and their methods of use and processes of manufacture are provided. This invention provides novel modifications of mannose 6-phosphate, galactose, and talose for use in extracellular protein degradation. The invention also provides new extracellular protein degraders that use a mixture of mannose 6-phosphate, galactose, and talose based targeting ligands. These compounds can be used for intracorporeal therapeutic bloodpheresis.

### I. Mannose-Based Extracellular Protein Degraders of the Present Invention

In certain embodiments the extracellular protein degrading compound is selected from:

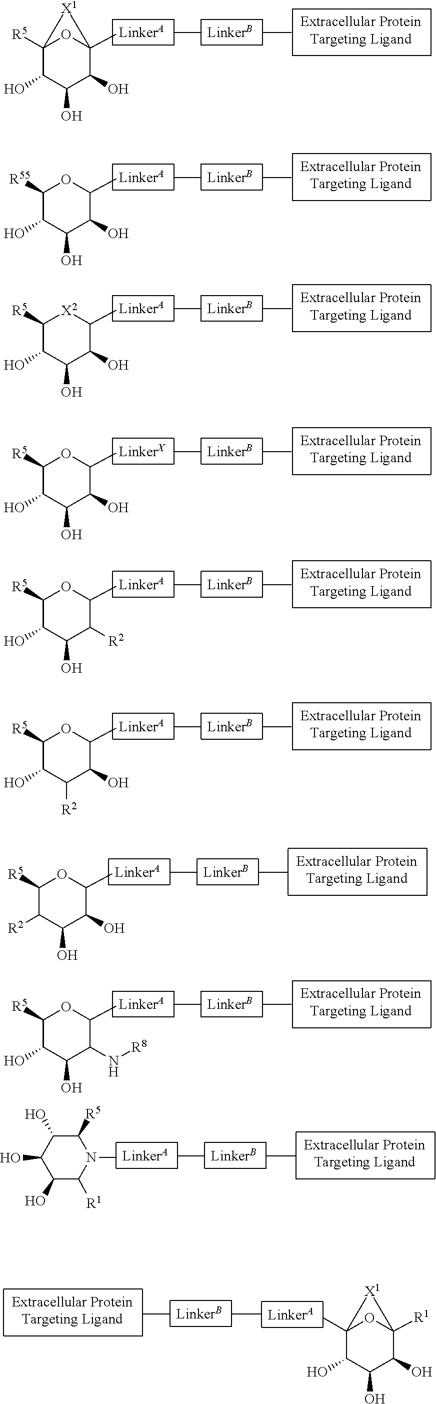

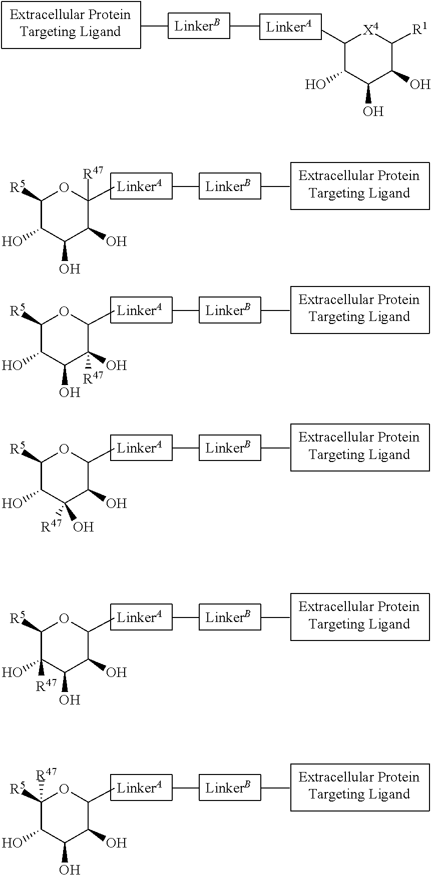

or a pharmaceutically acceptable salt thereof.

In other embodiments the extracellular protein degrading compound is a bi- (Formula II) or tri- (Formula III) version of the above structures. Non-limiting examples of compounds of Formula II include:

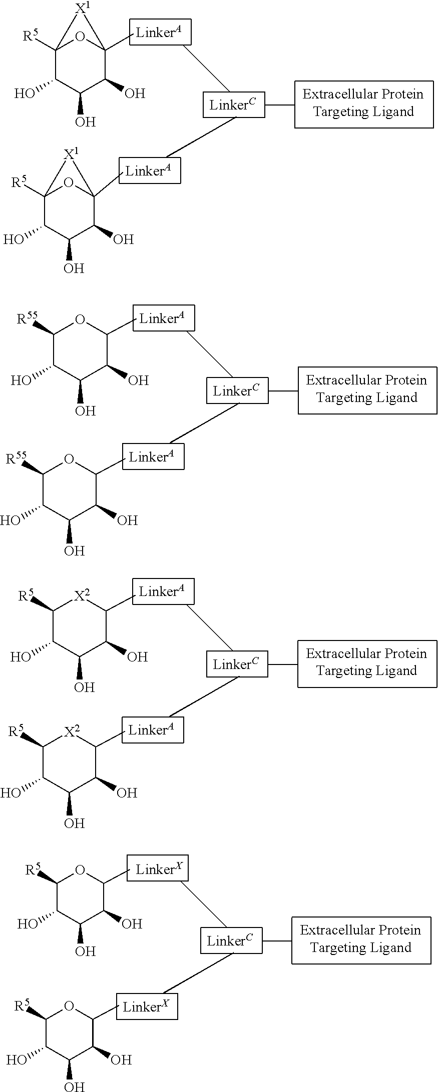

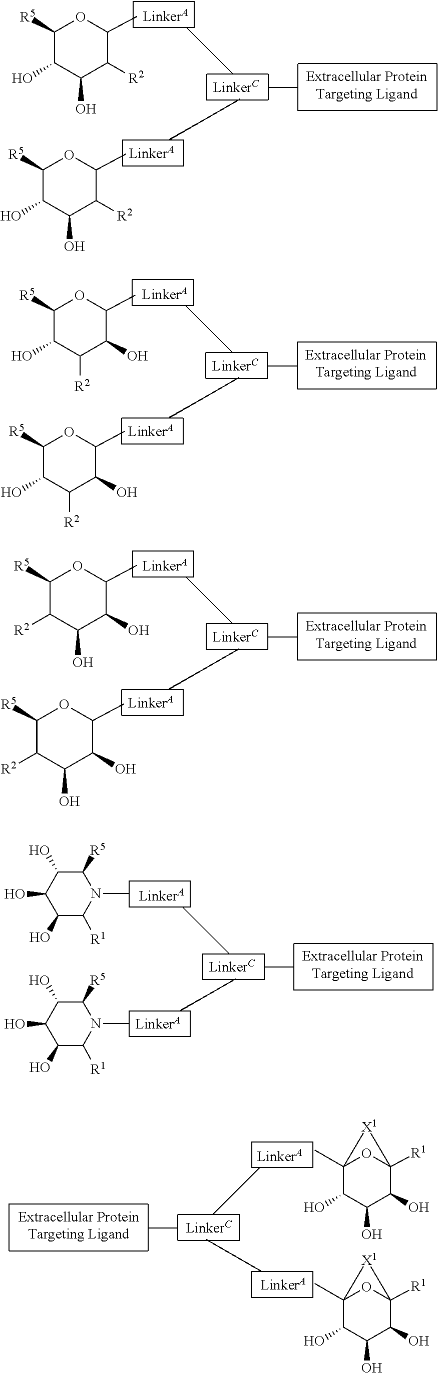

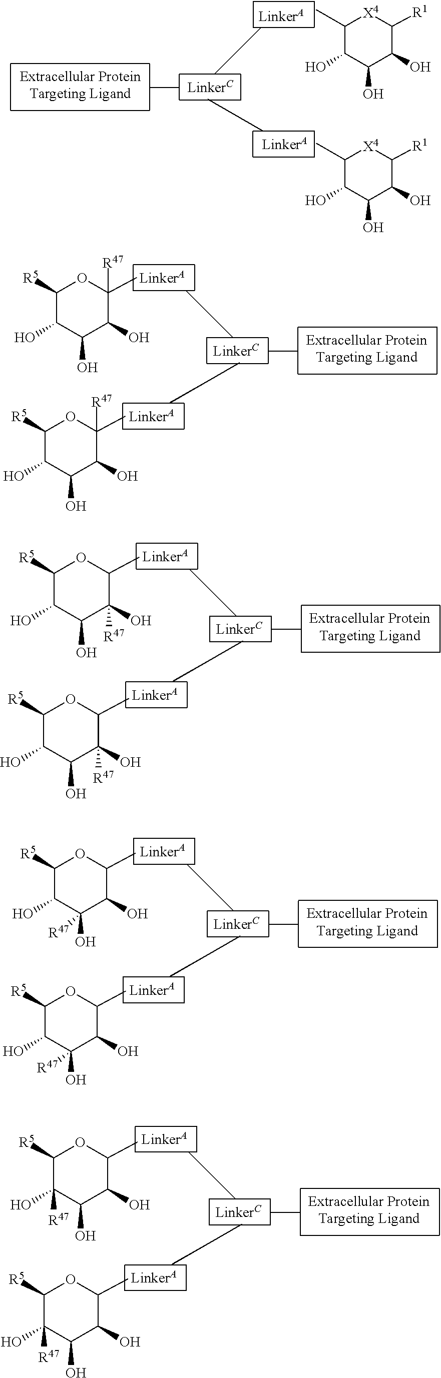

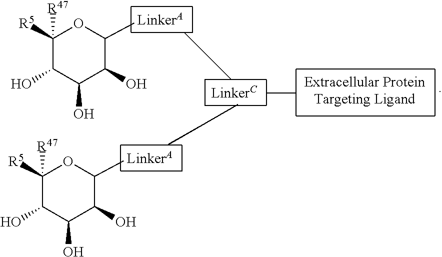

In certain embodiments, the extracellular protein degrading compound is of Formula III. Non-limiting examples of compounds of Formula III include.

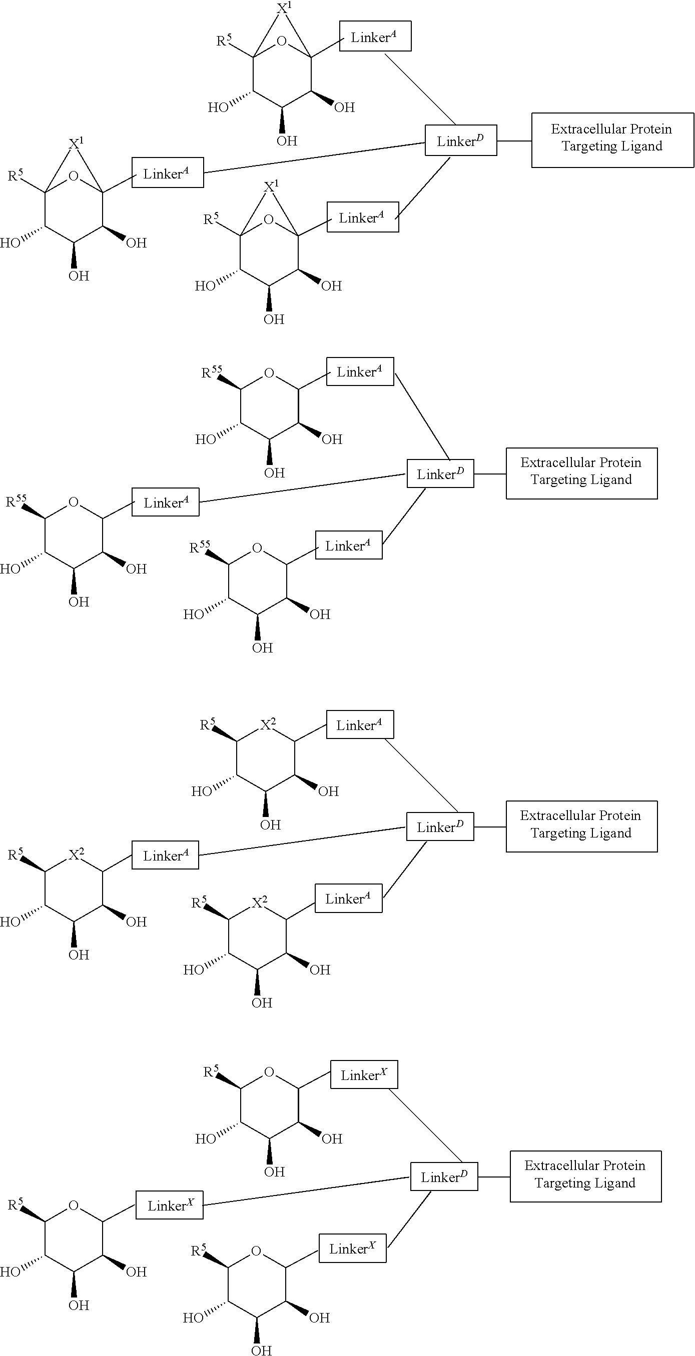

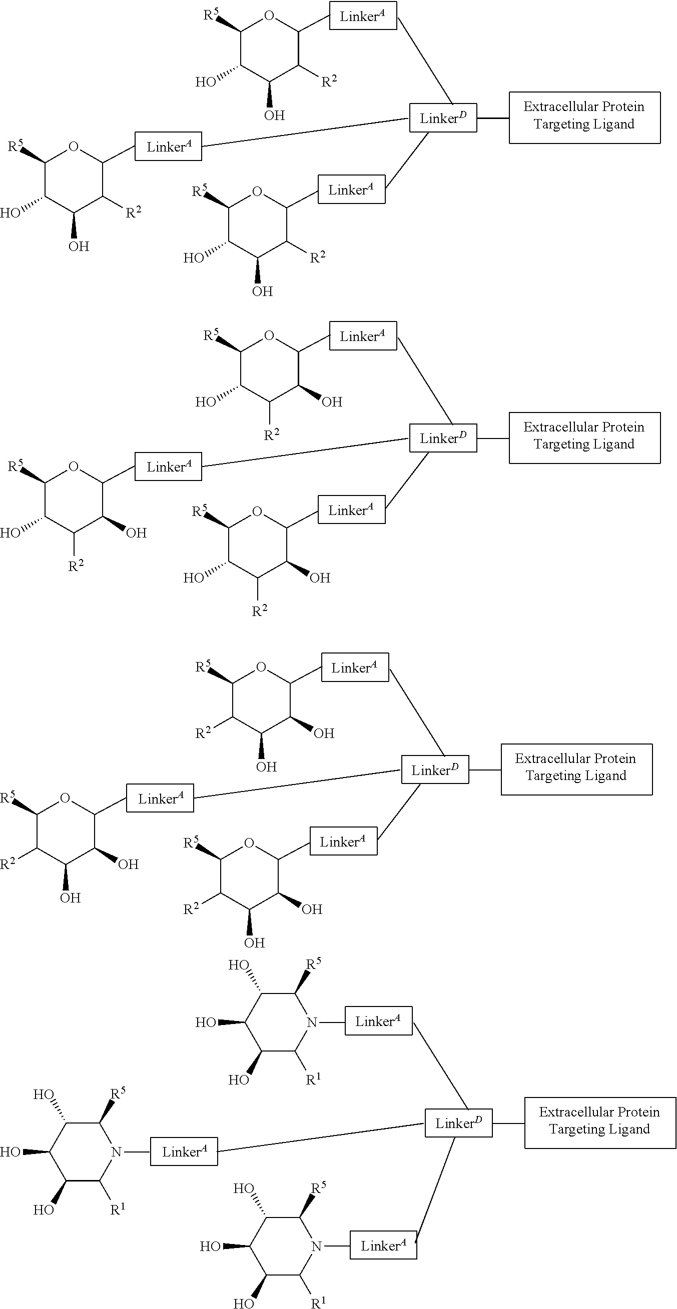

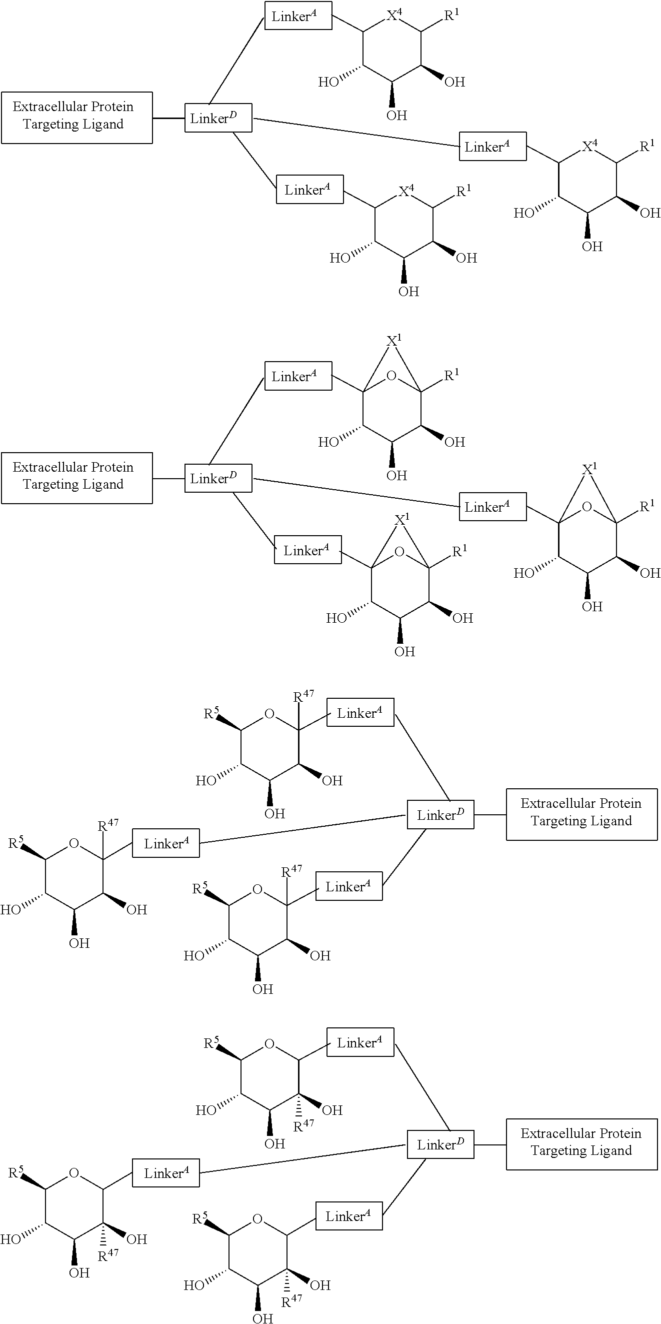

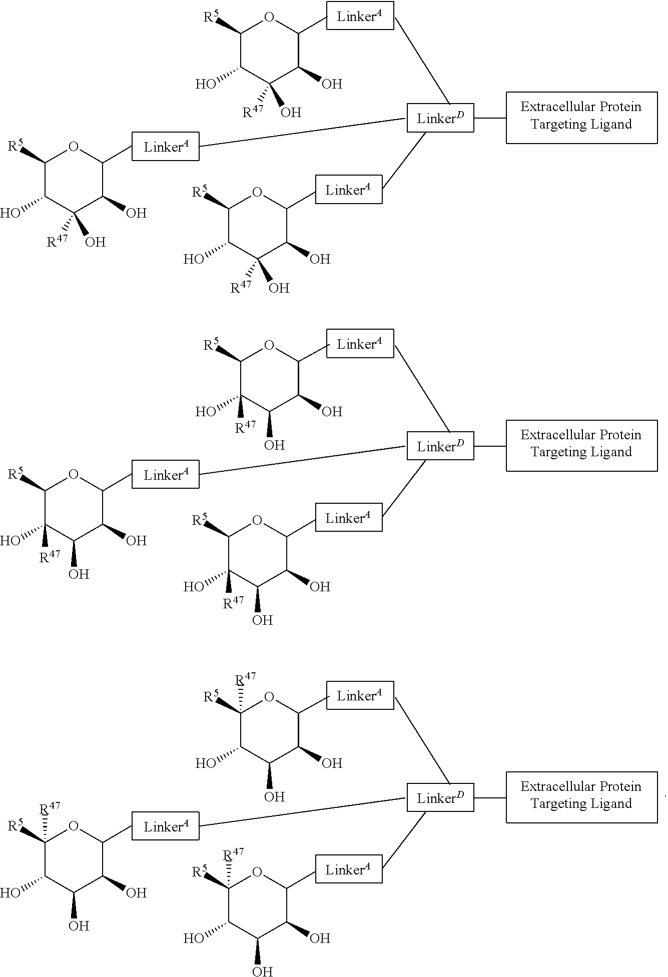

As used in the embodiments here, xx is independently selected from 0, 1, 2, 3, 4, 5, 6, 7, 8, 9, 10, 11, 12, 13, 14, 15, 16, 17, 18, 19, 20, 21, 22, 23, 24, and 25.
or each xx is independently selected from 0, 1, 2, 3, 4, 5, 6, 7, 8, 9, 10, 11, 12, 13, 14, 15, 16, 17, 18, 19, and 20.

As used in the embodiments here, yy is independently selected from 0, 1, 2, 3, 4, 5, 6, 7, 8, 9, 10, 11, 12, 13, 14, 15, 16, 17, 18, 19, 20, 21, 22, 23, 24, and 25.
or each yy is independently selected from 0, 1, 2, 3, 4, 5, 6, 7, 8, 9, 10, 11, 12, 13, 14, 15, 16, 17, 18, 19, and 20.

In certain embodiments the compound of the present invention is selected from:

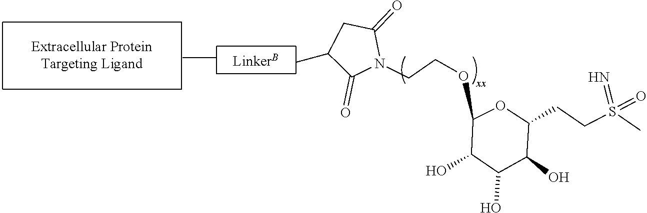

In certain embodiments the compound of the present invention is selected from:

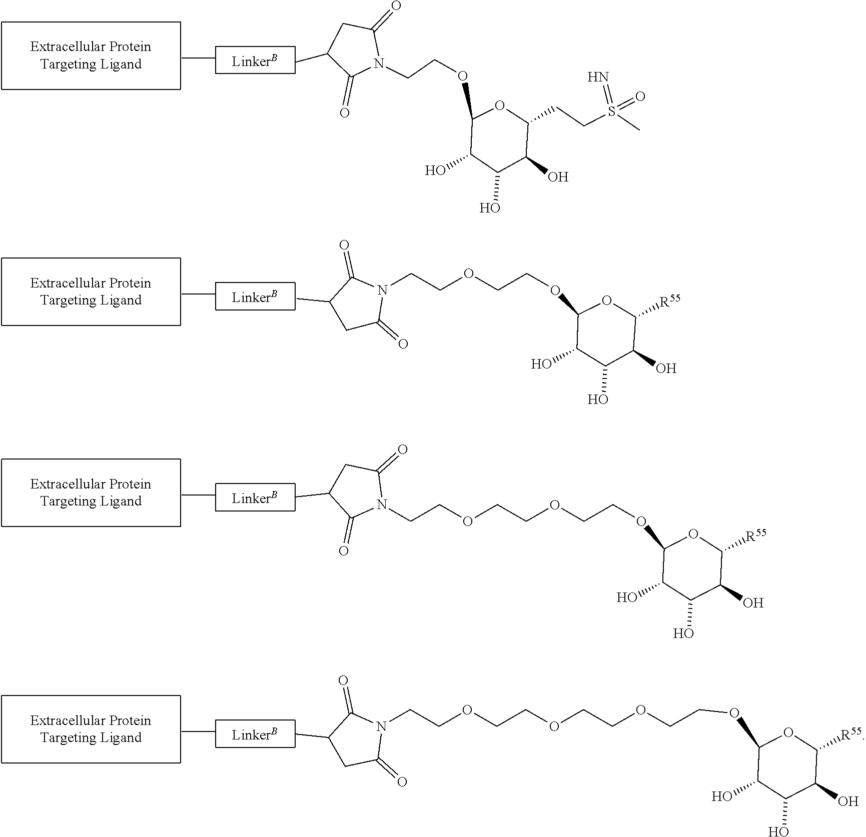

In certain embodiments the compound of the present invention is selected from:

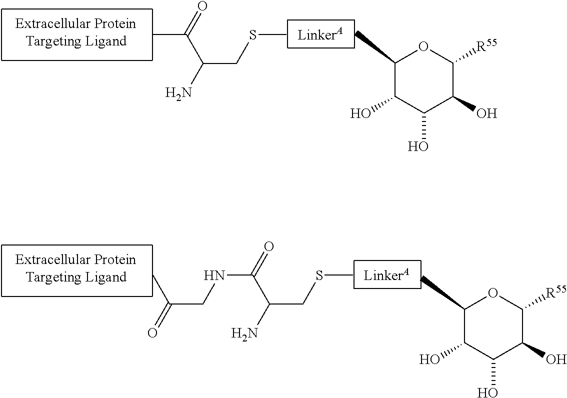

In certain embodiments the compound of the present invention is selected from:

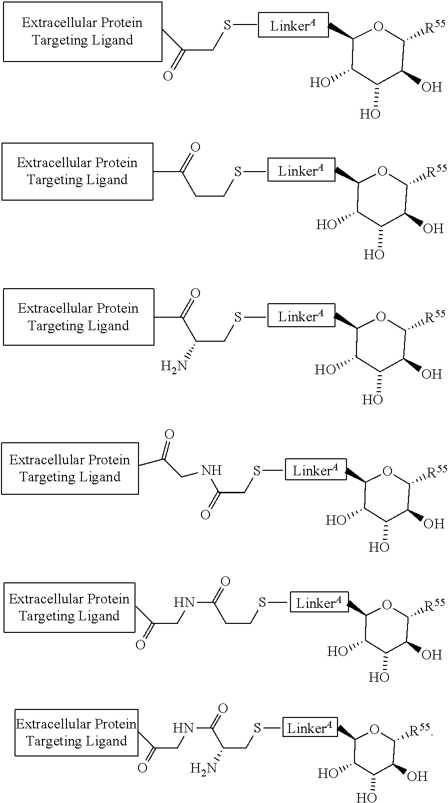

In certain embodiments the compound of the present invention is selected from:

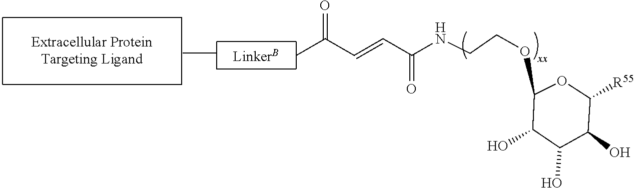

In certain embodiments the compound of the present invention is selected from:

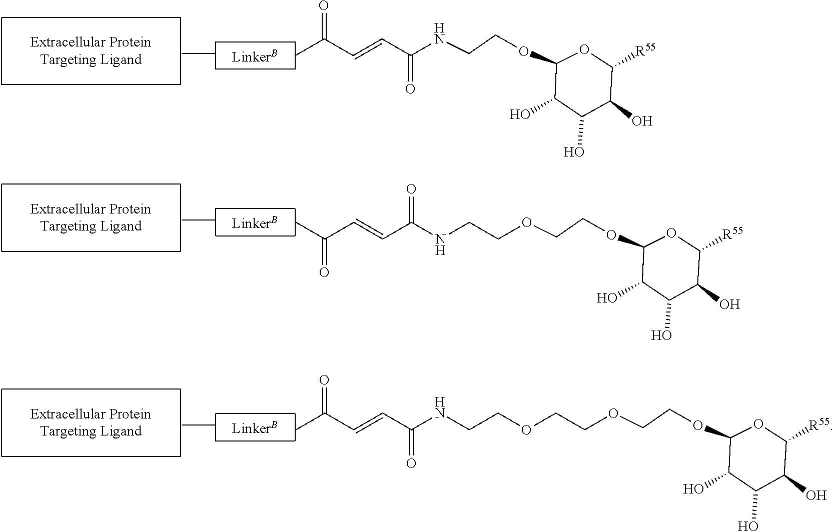

In certain embodiments the compound of the present invention is selected from:

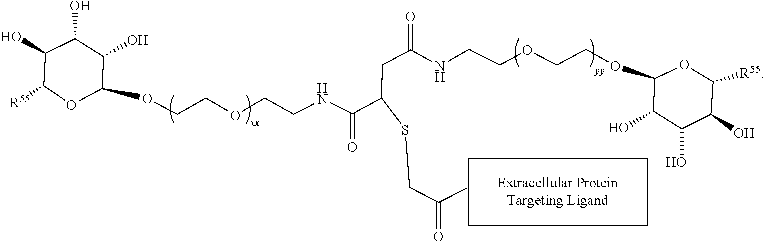

In one aspect of the present invention an Extracellular Protein degrading compound is provided wherein the mannose 6-phosphate receptor ligand or ASGPR ligand is a ligand described herein. In this aspect the mannose 6-phosphate receptor ligand or ASGPR ligand is linked in either the C1 or C5 position to form a degrading compound, for example, when the mannose 6-phosphate receptor ligand is

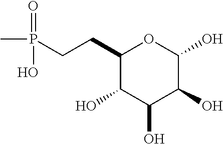 [SMILES: CP(=O)(O)CC[C@H]1O[C@H](O)[C@@H](O)[C@@H](O)[C@@H]1O]

then non-limiting examples of mannose 6-phosphate receptor binding compounds contemplated by this aspect include:

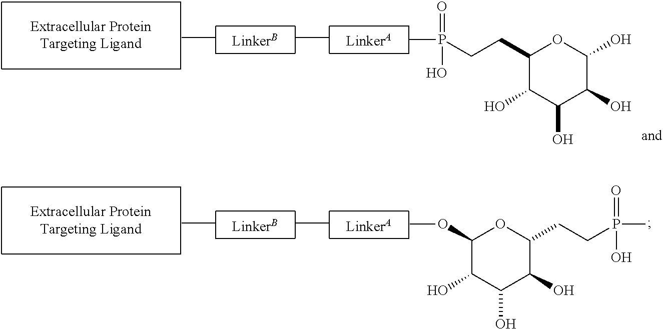

or the bi- or tri- version thereof or a pharmaceutically acceptable salt thereof.

In certain embodiments, a mannose 6-phosphate receptor ligand useful for incorporation into a compound of the present invention is selected from:

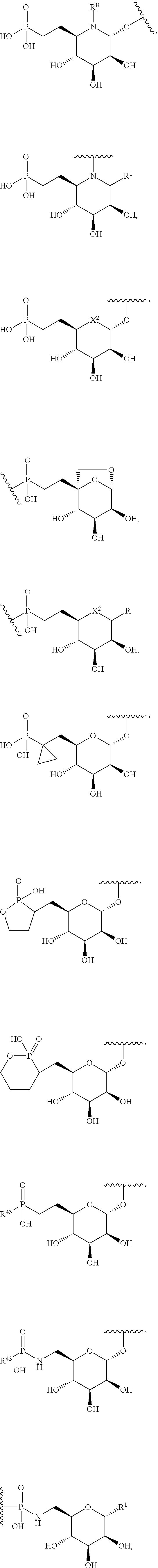

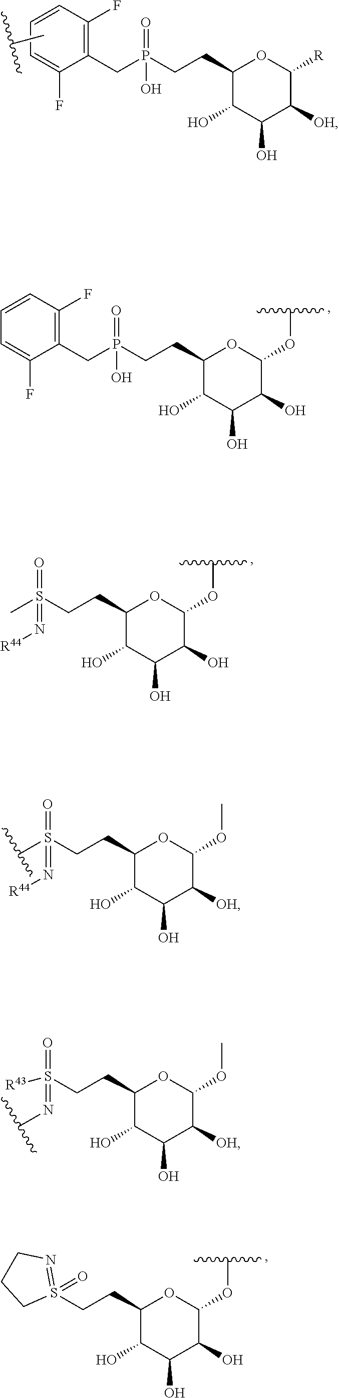

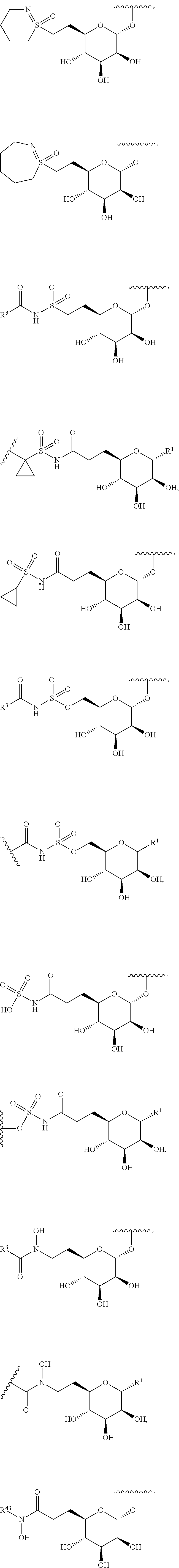

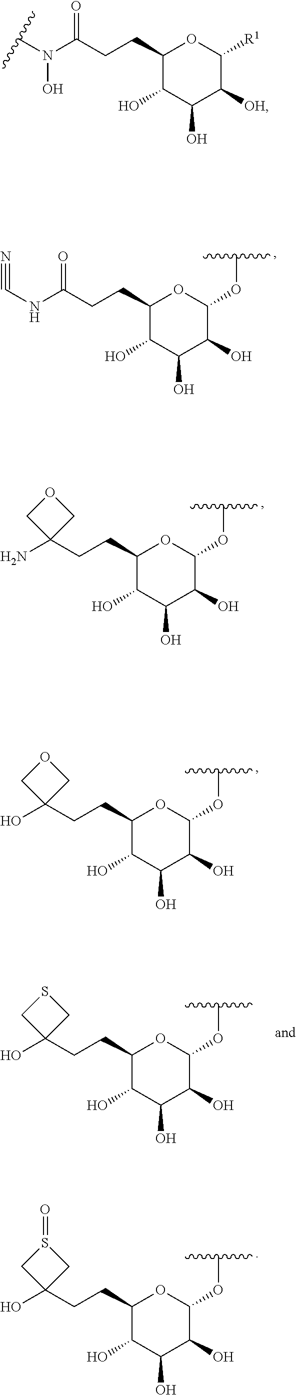

In certain embodiments, the mannose 6-phosphate is selected from:

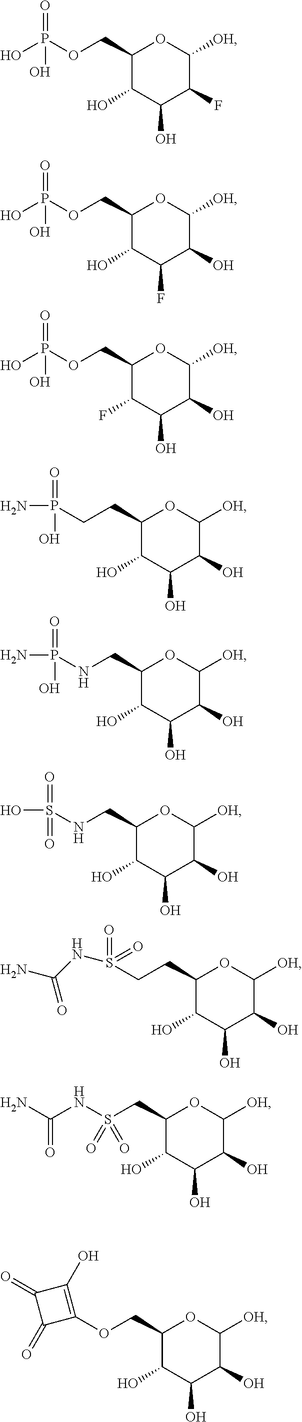

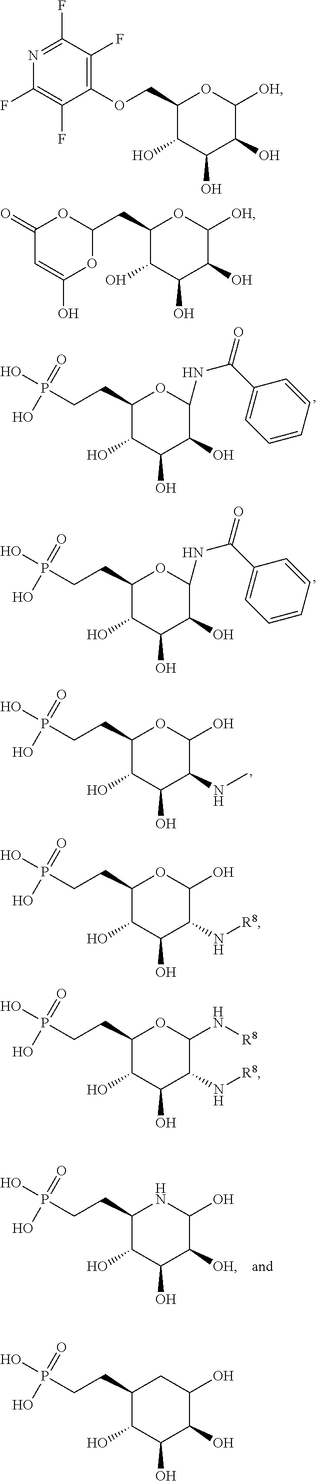

In certain embodiments, the tricyclic linker is selected from:

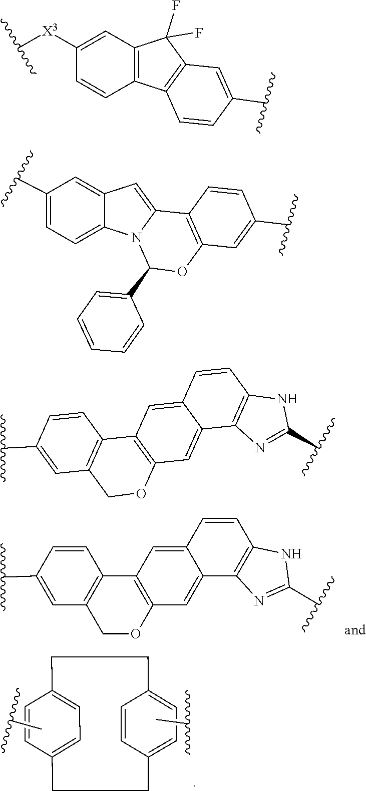

In certain embodiments, the spirocyclic or bicyclic linker is selected from:

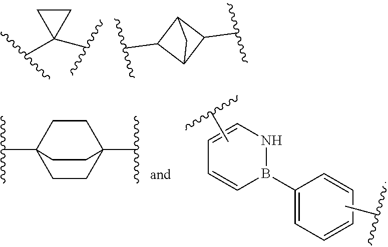

In certain embodiments, the Mannose 6-Phosphate Ligand^{A} is selected from:

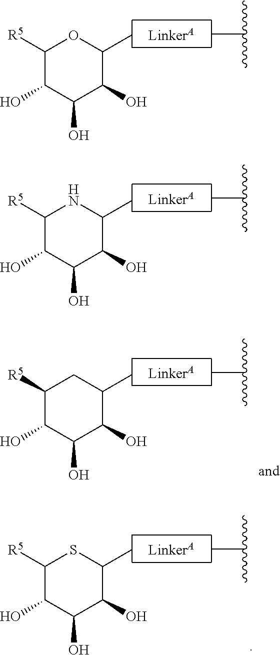

In certain embodiments, the Mannose 6-Phosphate Ligand^{A} is selected from:

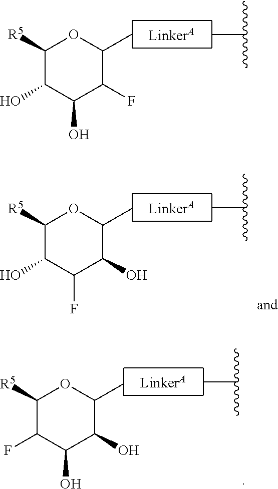

In certain embodiments, the Mannose 6-Phosphate Ligand^{A} is selected from:

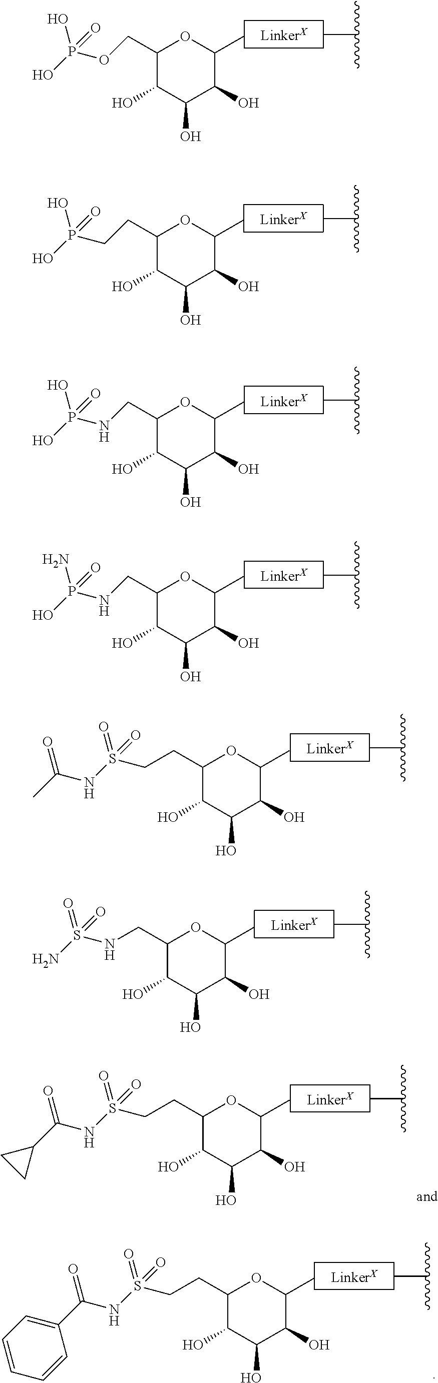

In certain embodiments, the Mannose 6-Phosphate Ligand^{A} is selected from:

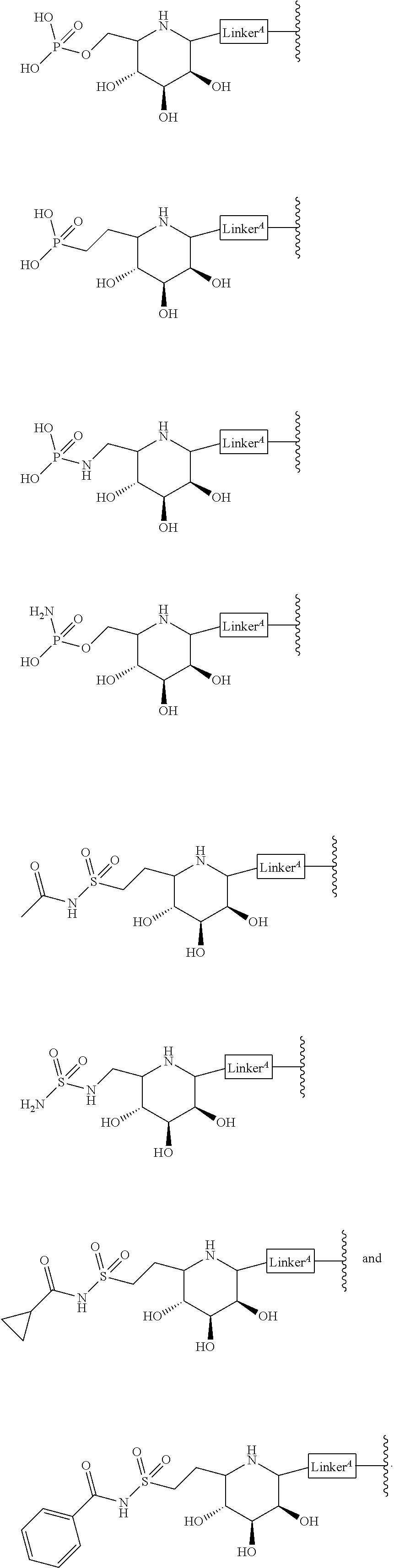

In certain embodiments, the Mannose 6-Phosphate Ligand^{A} is selected from:

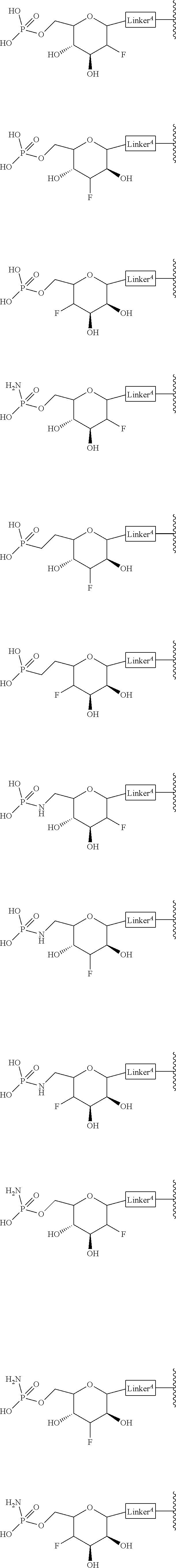

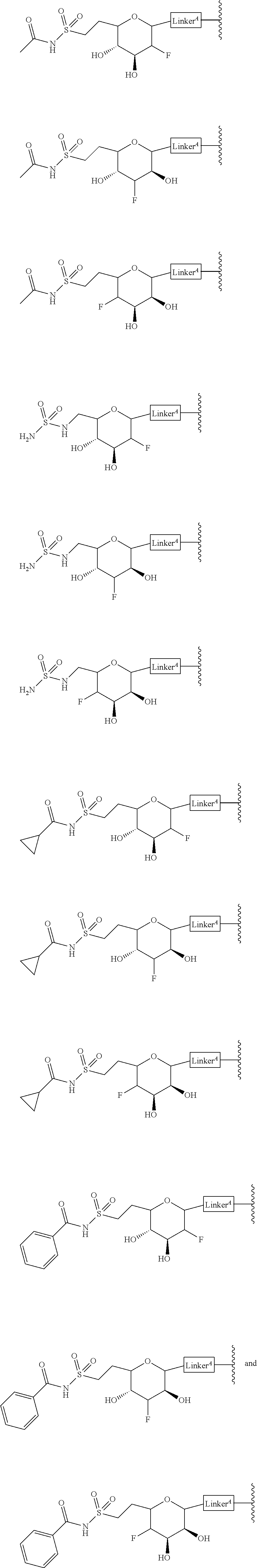

In certain embodiments, the Mannose 6-Phosphate Ligand^{A} is selected from:

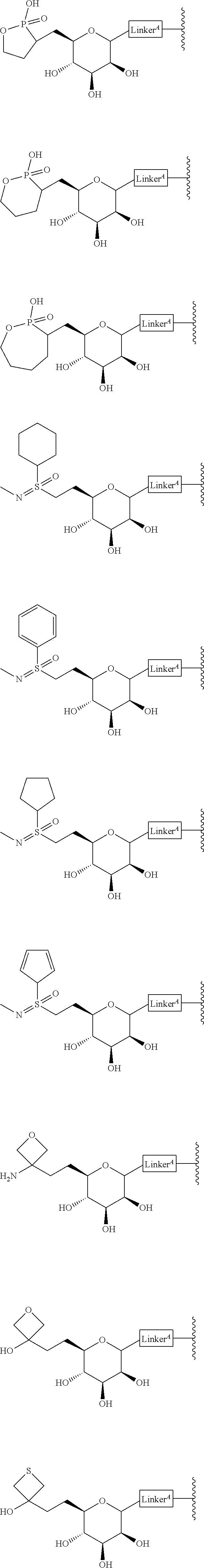

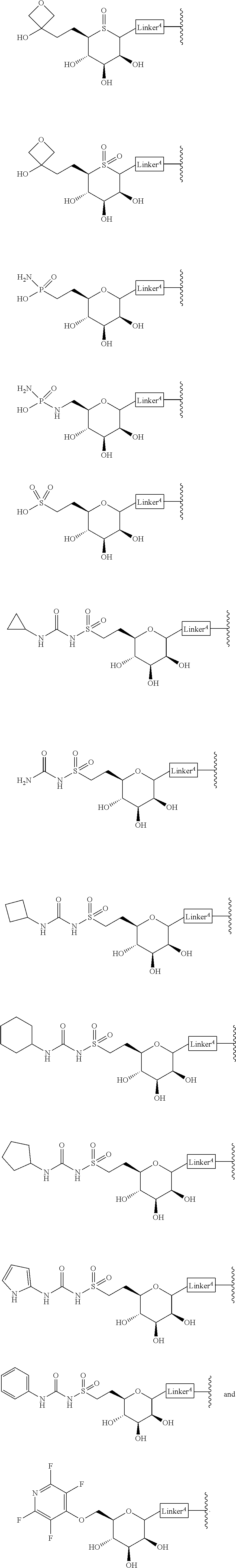

In certain embodiments, the Mannose 6-Phosphate Ligand^{A} is selected from:

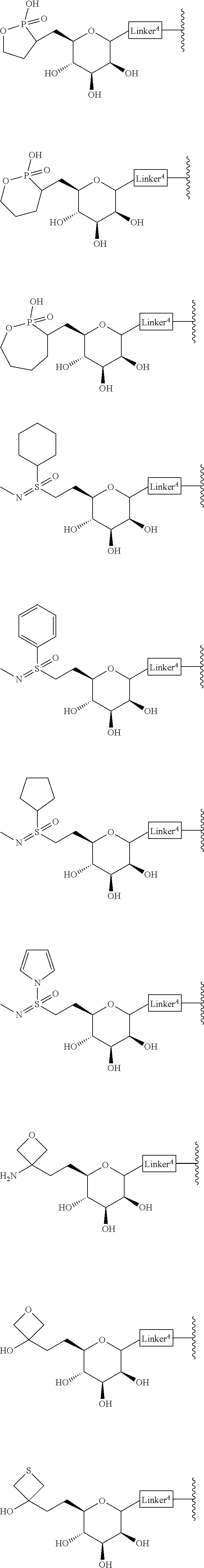

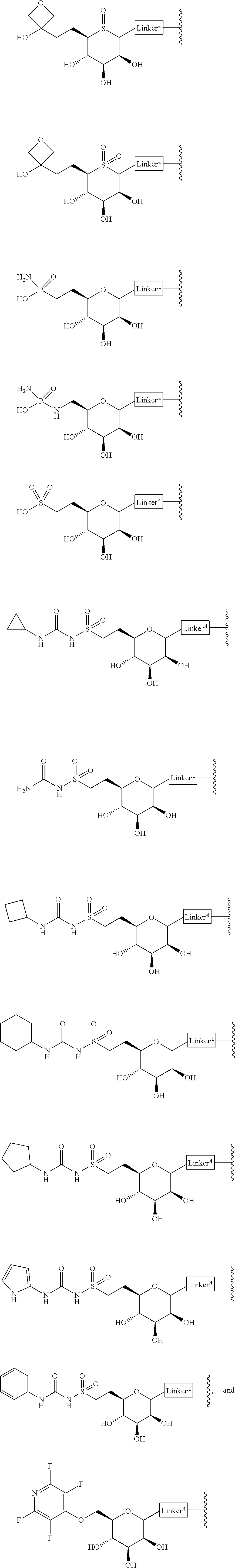

In certain embodiments, the Mannose 6-Phosphate Ligand^{A} is selected from:

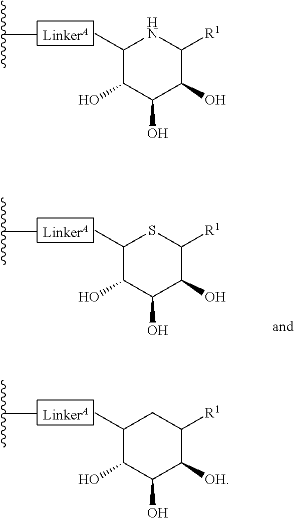

In certain alternative embodiments the Mannose 6-Phosphate Ligand^{A} is selected from:

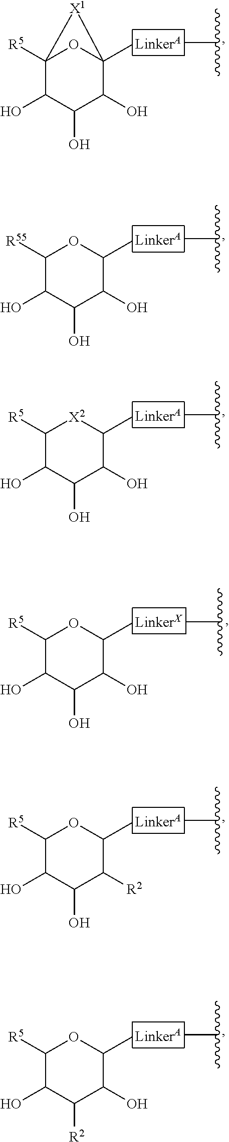

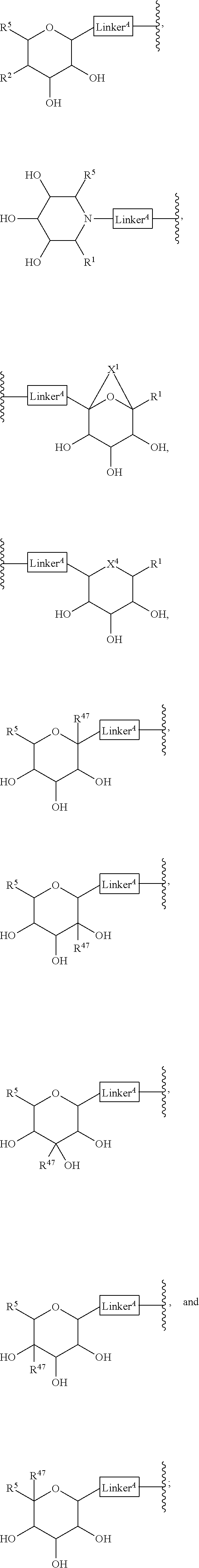

In certain alternative embodiments Mannose 6-Phosphate Ligand^{B} is selected from: Mannose 6-Phosphate Ligand^{A} and OH

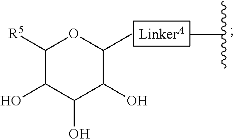

In certain alternative embodiments ASGPR Ligand^{B} is selected from:

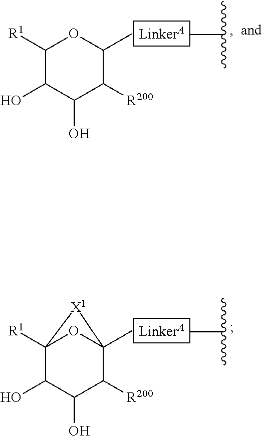

In certain embodiments Mannose 6-Phosphate Ligand^{A} is selected from:

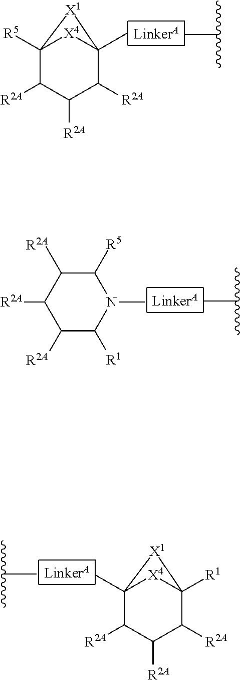

wherein
R^{2A} is selected from R² and O;

In certain embodiments Mannose 6-Phosphate Ligand^{A} is selected from:

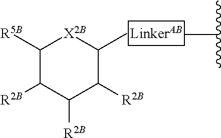

wherein
each R^{2B} is independently selected from R² and OH;
X^{2B} is selected from X² and O;
R^{5B} is selected from R⁵ and R⁵⁵;
each R^{47B} is independently selected from R⁴⁷ and H;
Linker^{AB} is selected from Linker^{A} and Linker^{X}; and
wherein at least one of the following criteria is met:
   a. at least one R^{2B} is R²;
   b. X^{2B} is X²;
   c. R^{5B} is R⁵⁵;
   d. Linker^{AB} is Linker^{X}; or
   e. at least one R^{47B} is R⁴⁷.

### II. Galactose and Talose-Based Extracellular Protein Degraders of the Present Invention

In certain embodiments, the extracellular protein degrading compound is selected from Formula IV. Non-limiting examples of compounds include:

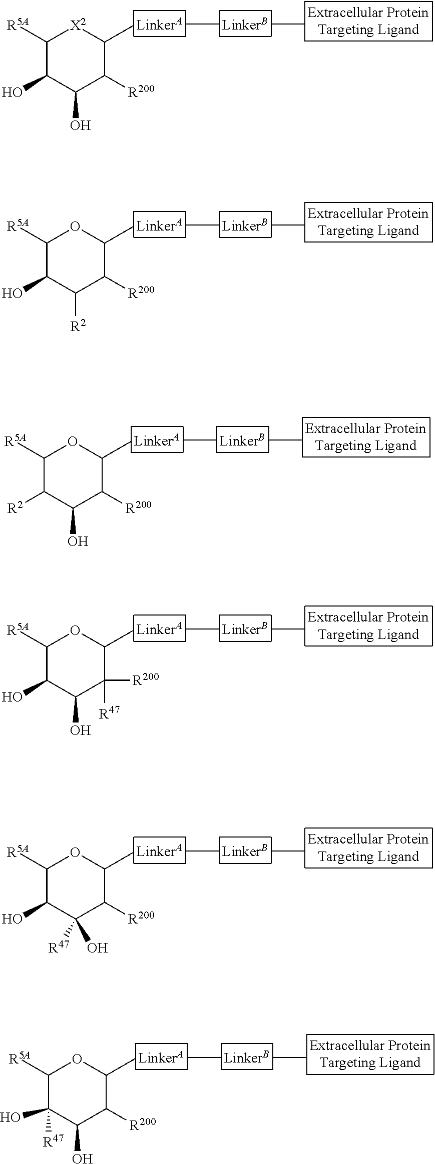

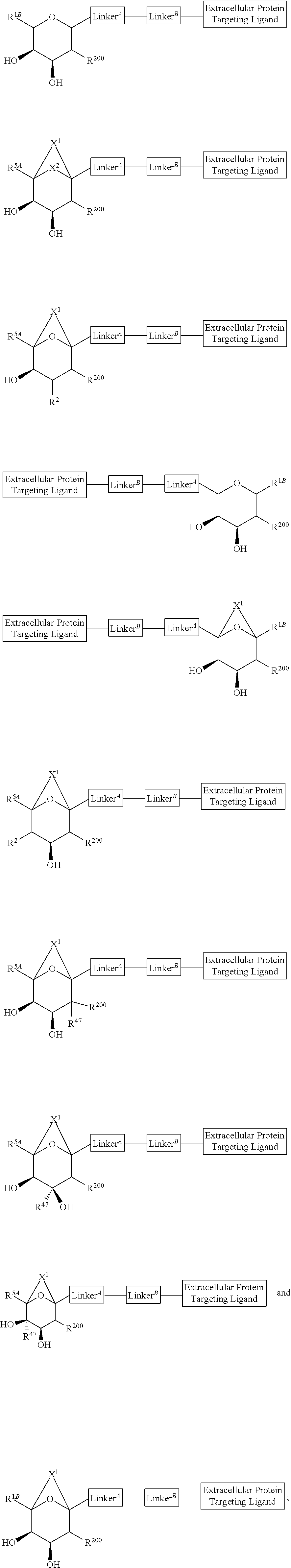

or a pharmaceutically acceptable salt thereof.

In other embodiments, the extracellular protein degrading compound is selected from Formula V. Non-limiting examples of compounds include:

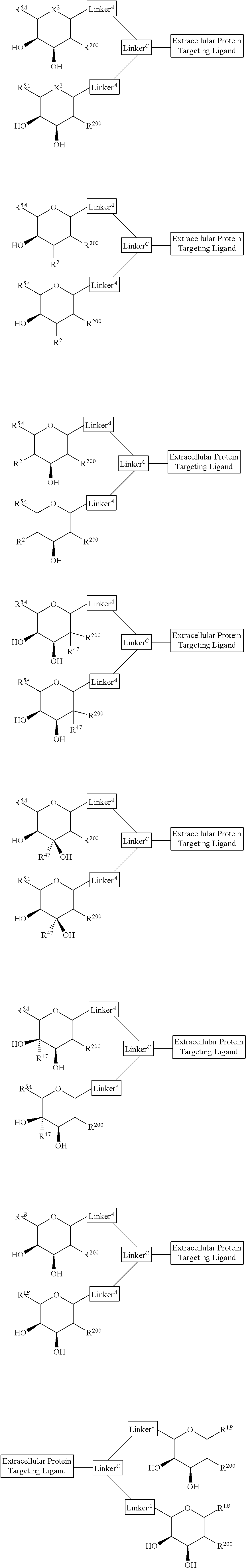

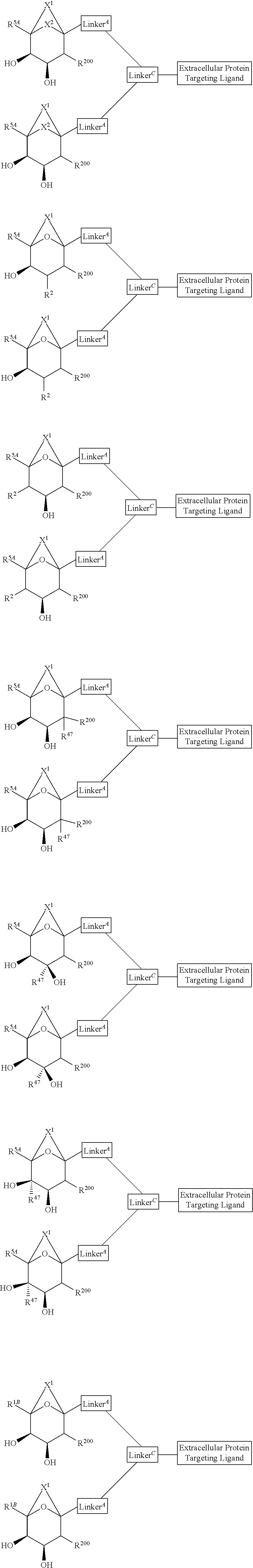

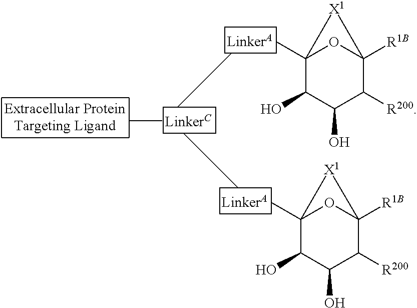

In certain embodiments, the extracellular protein degrading compound is selected from Formula VI. Non-limiting examples of compounds include:

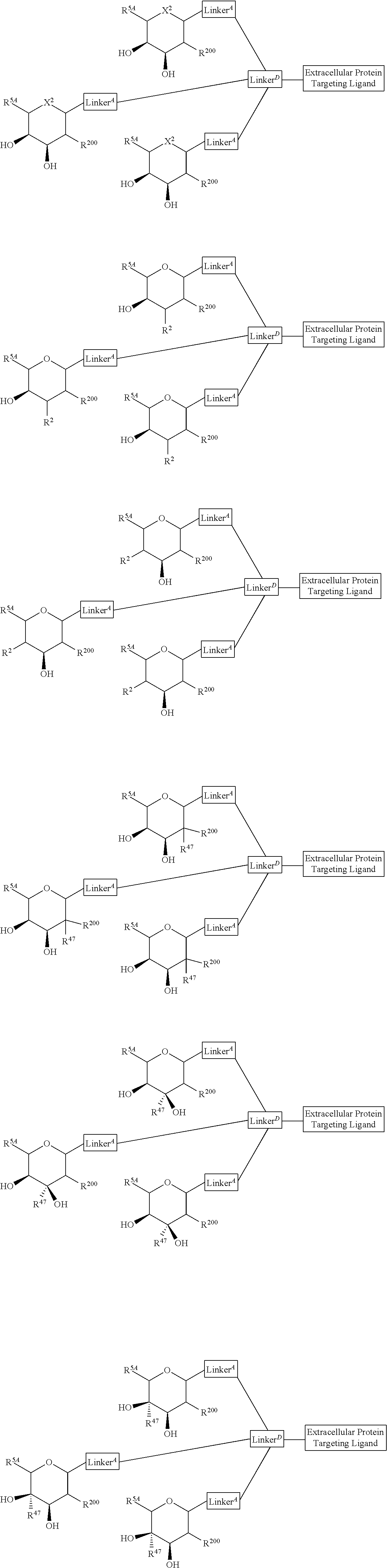

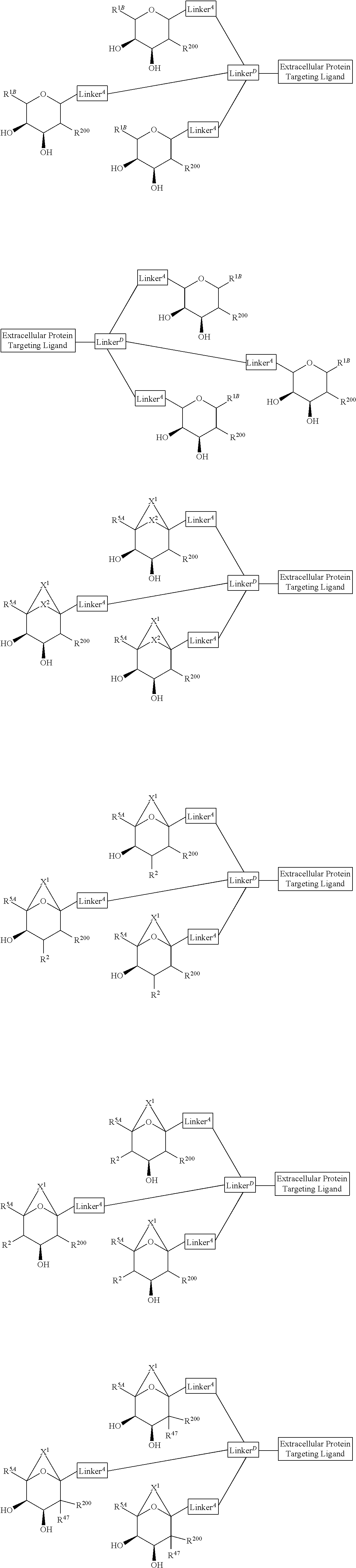

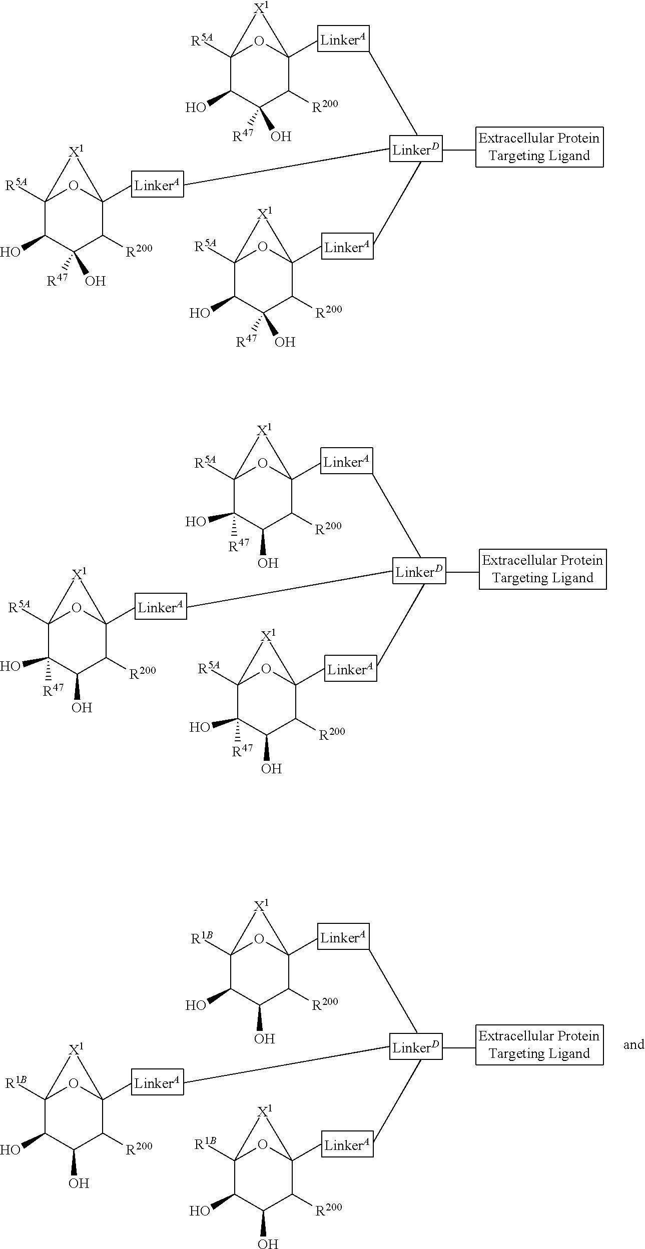

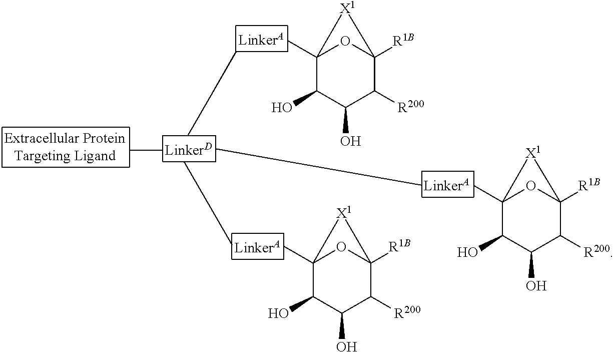

In certain embodiments, the ASGPR Ligand^{A} is selected from:

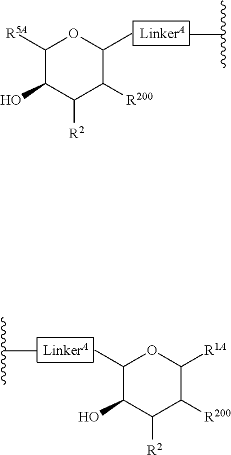

In certain embodiments, the ASGPR Ligand^{A} is selected from:

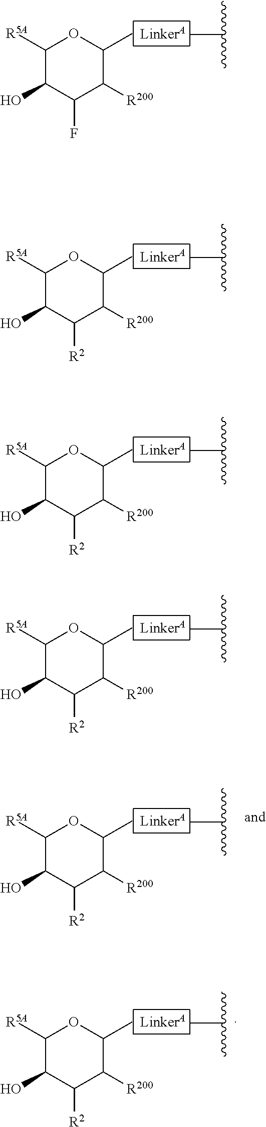

In certain embodiments, the ASGPR Ligand^{A} is selected from:

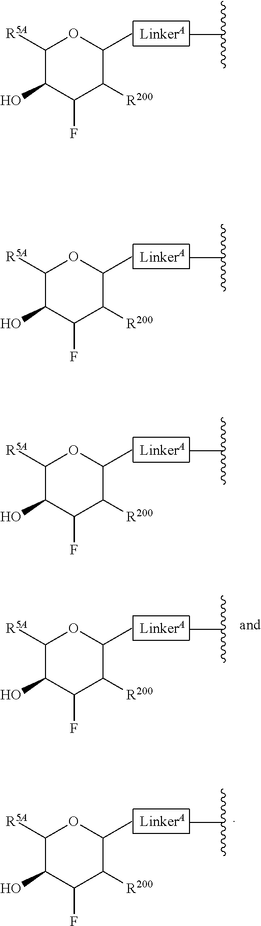

In certain embodiments ASGPR Ligand^{A} is selected from

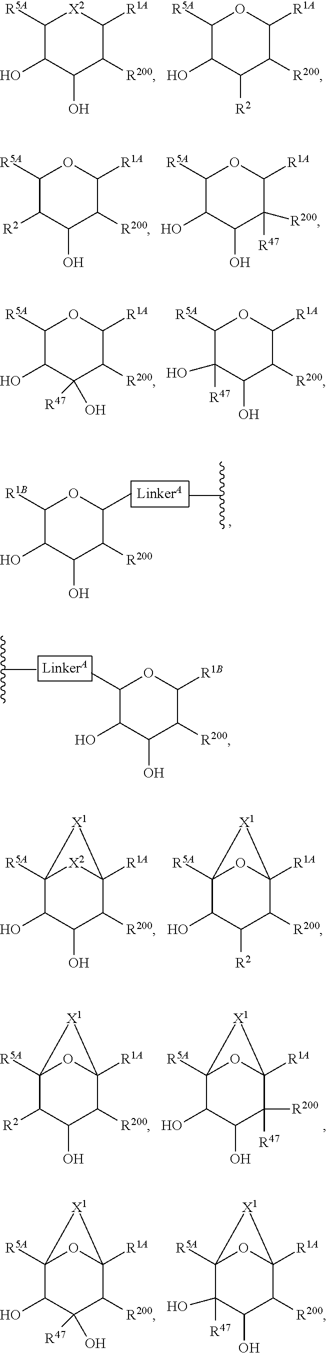

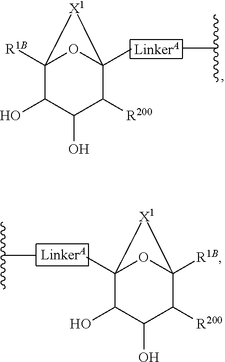

### III. Mixed Degraders of the Present Invention

In certain aspects of the present invention, an extracellular protein degrader of Formula VII, Formula VIII, or Formula IX is provided that includes a mixture of Mannose 6-Phosphate Ligands and ASGPR Ligands:

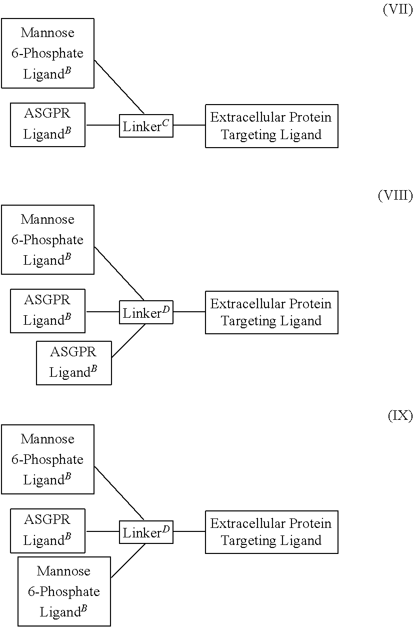

or a pharmaceutically acceptable salt thereof.

### IV. Non-Limiting Embodiments of the Present Invention

### Embodiments of R¹

In certain embodiments R¹ is hydrogen.

In certain embodiments R¹ is

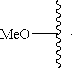 [SMILES: *OC]

In certain embodiments R¹ is

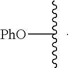 [SMILES: *Oc1ccccc1]

In certain embodiments R¹ is

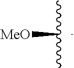

In certain embodiments R¹ is

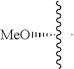

In certain embodiments R¹ is

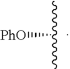

In certain embodiments R¹ is

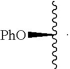

In certain embodiments R¹ is heteroalkyl optionally substituted with 1, 2, 3, or 4 substituents.

In certain embodiments R¹ is C₀-C₆alkyl-cyano optionally substituted with 1, 2, 3, or 4 substituents.

In certain embodiments R¹ is alkyl optionally substituted with 1, 2, 3, or 4 substituents.

In certain embodiments R¹ is alkenyl optionally substituted with 1, 2, 3, or 4 substituents.

In certain embodiments R¹ is alkynyl optionally substituted with 1, 2, 3, or 4 substituents.

In certain embodiments R¹ is haloalkyl optionally substituted with 1, 2, 3, or 4 substituents.

In certain embodiments R¹ is F.

In certain embodiments R¹ is Cl.

In certain embodiments R¹ is Br.

In certain embodiments R¹ is aryl optionally substituted with 1, 2, 3, or 4 substituents.

In certain embodiments R¹ is arylalkyl optionally substituted with 1, 2, 3, or 4 substituents.

In certain embodiments R¹ is heteroaryl optionally substituted with 1, 2, 3, or 4 substituents.

In certain embodiments R¹ is heteroarylalkyl optionally substituted with 1, 2, 3, or 4 substituents.

In certain embodiments R¹ is heterocycle optionally substituted with 1, 2, 3, or 4 substituents.

In certain embodiments R¹ is heterocycloalkyl optionally substituted with 1, 2, 3, or 4 substituents.

In certain embodiments R¹ is haloalkoxy optionally substituted with 1, 2, 3, or 4 substituents.

In certain embodiments R¹ is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³, each of which is optionally substituted with 1, 2, 3, or 4 substituents.

### Embodiments of R²

In certain embodiments R² is F.

In certain embodiments R² is SH.

In certain embodiments R² is NHR⁸.

In certain embodiments R² is OR⁴³.

### Embodiments of R²⁰⁰

In certain embodiments R²⁰⁰ is aryl optionally substituted with 1, 2, 3, or 4 substituents.

In certain embodiments R²⁰⁰ is heterocycle optionally substituted with 1, 2, 3, or 4 substituents.

In certain embodiments R²⁰⁰ is heteroaryl containing 1 or 2 heteroatoms independently selected from N, O, and S optionally substituted with 1, 2 3 or 4 substituents.

In certain embodiments R²⁰⁰ is selected from

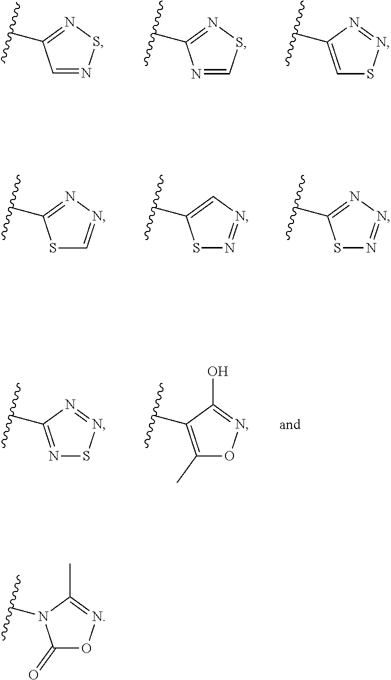

In certain embodiments R²⁰⁰ is heterocycle optionally substituted with 1, 2, 3, or 4 substituents.

In certain embodiments R²⁰⁰ is —NR⁸—S(O)—R³ optionally substituted with 1, 2, 3, or 4 substituents.

In certain embodiments R²⁰⁰ is —NR⁸—C(S)—R³ optionally substituted with 1, 2, 3, or 4 substituents.

In certain embodiments R²⁰⁰ is —NR⁸—S(O)(NR⁶)—R³ optionally substituted with 1, 2, 3, or 4 substituents.

In certain embodiments R²⁰⁰ is —N═S(O)(R³)₂ optionally substituted with 1, 2, 3, or 4 substituents.

In certain embodiments R²⁰⁰ is —NR⁸C(O)NR⁹S(O)₂R³ optionally substituted with 1, 2, 3, or 4 substituents.

In certain embodiments R²⁰⁰ is —NR⁸—S(O)₂—R¹⁰ optionally substituted with 1, 2, 3, or 4 substituents.

In certain embodiments R²⁰⁰ is —NR⁸—C(NR⁶)—R³ optionally substituted with 1, 2, 3, or 4 substituents.

In certain embodiments R²⁰⁰ is hydrogen.

In certain embodiments R²⁰⁰ is R¹⁰.

In certain embodiments R²⁰⁰ is alkyl-C(O)—R³.

In certain embodiments R²⁰⁰ is —C(O)—R³.

In certain embodiments R²⁰⁰ is alkyl.

In certain embodiments R²⁰⁰ is haloalkyl.

In certain embodiments R²⁰⁰ is —OC(O)R³.

In certain embodiments R²⁰⁰ is —NR⁸—C(O)R¹⁰.

In certain embodiments R²⁰⁰ is alkenyl optionally substituted with 1, 2, 3, or 4 substituents.

In certain embodiments R²⁰⁰ is allyl optionally substituted with 1, 2, 3, or 4 substituents.

In certain embodiments R²⁰⁰ is alkynyl optionally substituted with 1, 2, 3, or 4 substituents.

In certain embodiments R²⁰⁰ is —NR⁶-alkenyl optionally substituted with 1, 2, 3, or 4 substituents.

In certain embodiments R²⁰⁰ is —O-alkenyl optionally substituted with 1, 2, 3, or 4 substituents.

In certain embodiments R²⁰⁰ is —NR⁶-alkynyl optionally substituted with 1, 2, 3, or 4 substituents.

In certain embodiments R²⁰⁰ is —NR⁶-heteroaryl optionally substituted with 1, 2, 3, or 4 substituents.

In certain embodiments R²⁰⁰ is —NR⁶-aryl optionally substituted with 1, 2, 3, or 4 substituents.

In certain embodiments R²⁰⁰ is —O-heteroaryl optionally substituted with 1, 2, 3, or 4 substituents.

In certain embodiments R²⁰⁰ is —O-aryl optionally substituted with 1, 2, 3, or 4 substituents.

In certain embodiments R²⁰⁰ is —O-alkynyl optionally substituted with 1, 2, 3, or 4 substituents.

In certain embodiments R²⁰⁰ is selected from

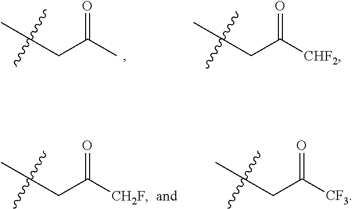

In certain embodiments R²⁰⁰ is selected from:

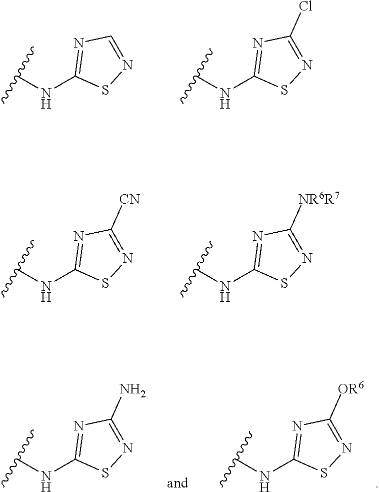

In certain embodiments R²⁰⁰ is selected from

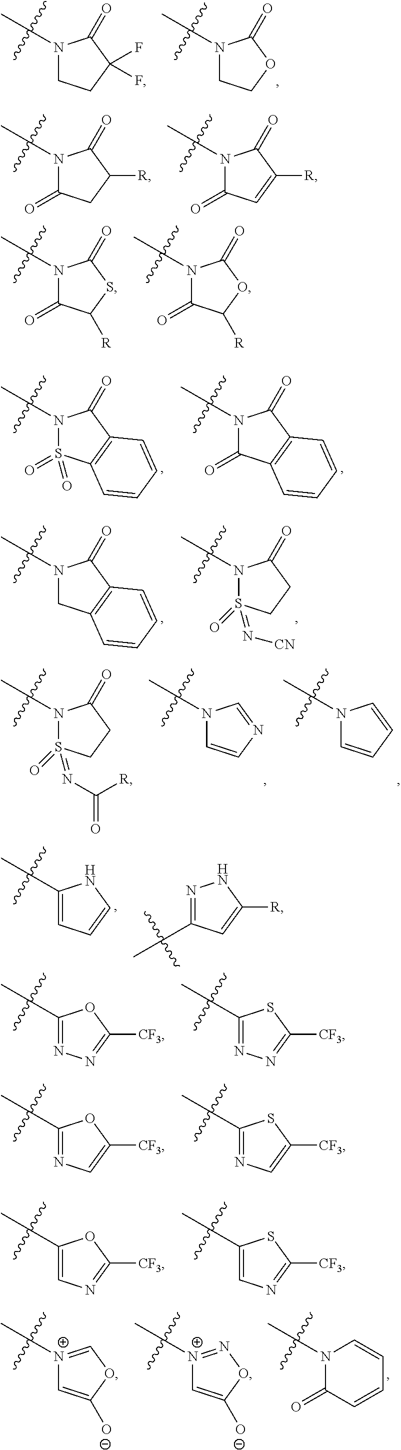

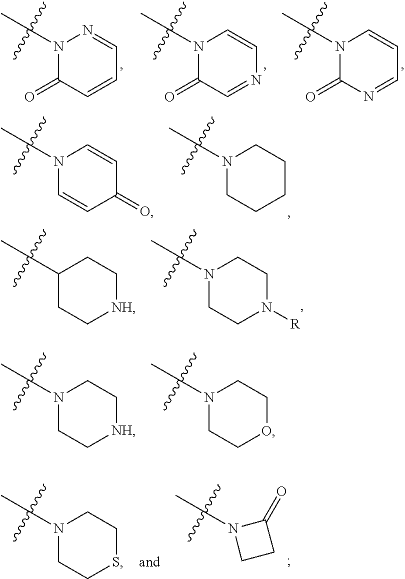

wherein R is an optional substituent as defined herein.

In certain embodiments R²⁰⁰ is selected from

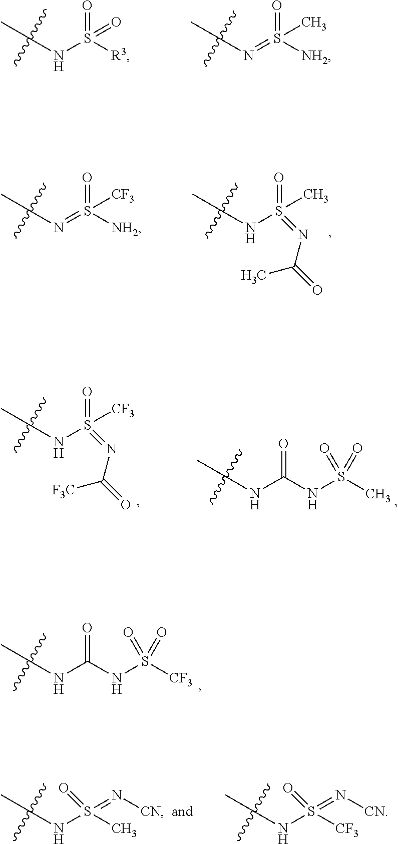

In certain embodiments R^{200 A} is selected from

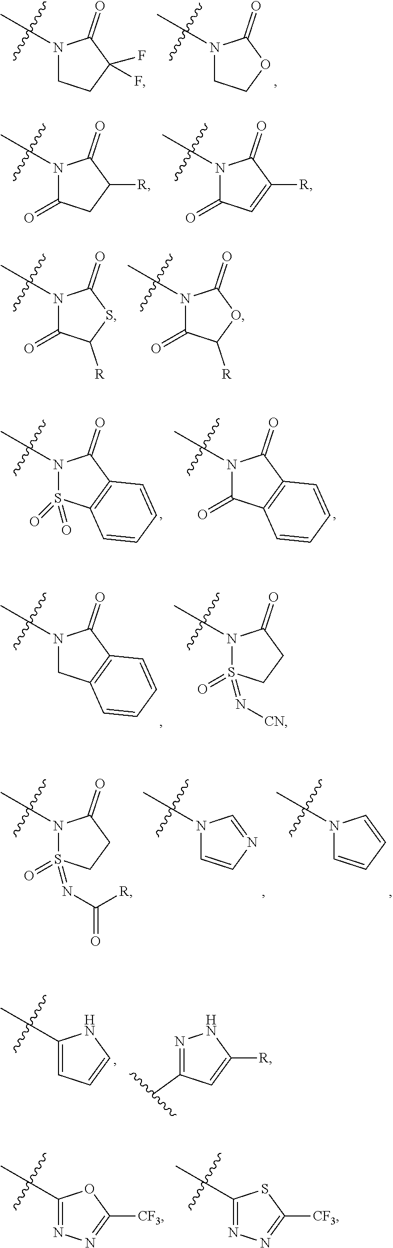

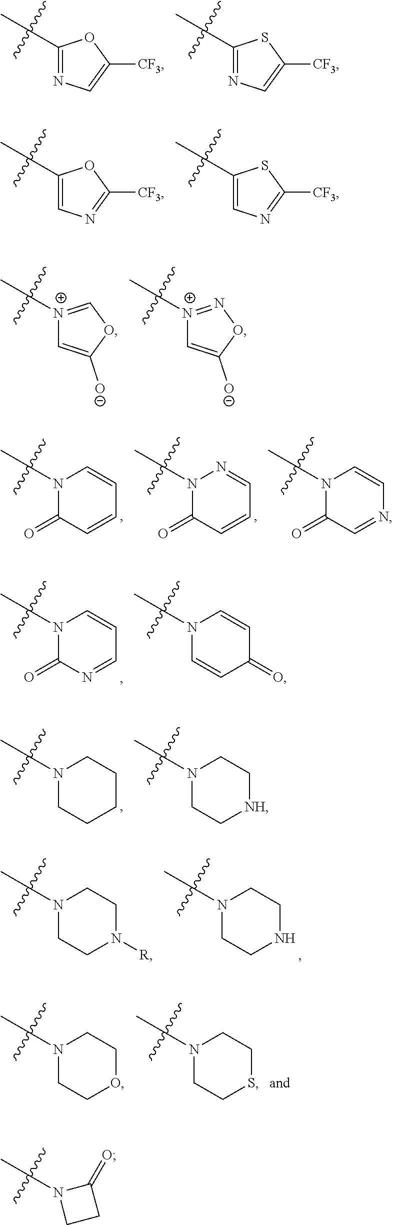

wherein R is an optional substituent as defined herein.

In certain embodiments R²⁰⁰ is selected from

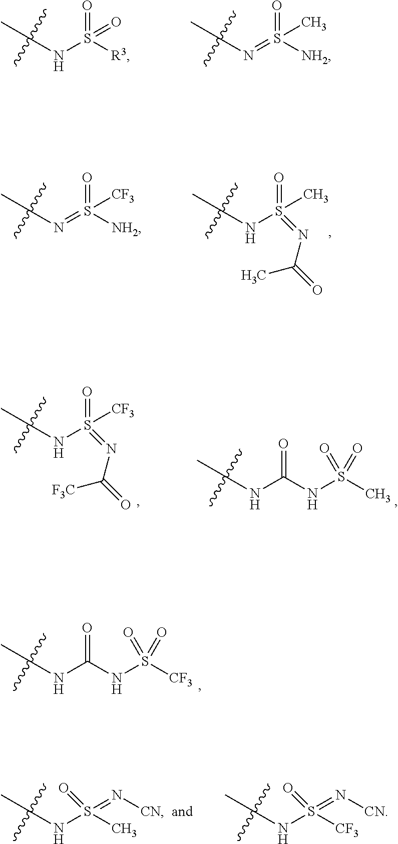

In certain embodiments, R²⁰⁰ is selected from

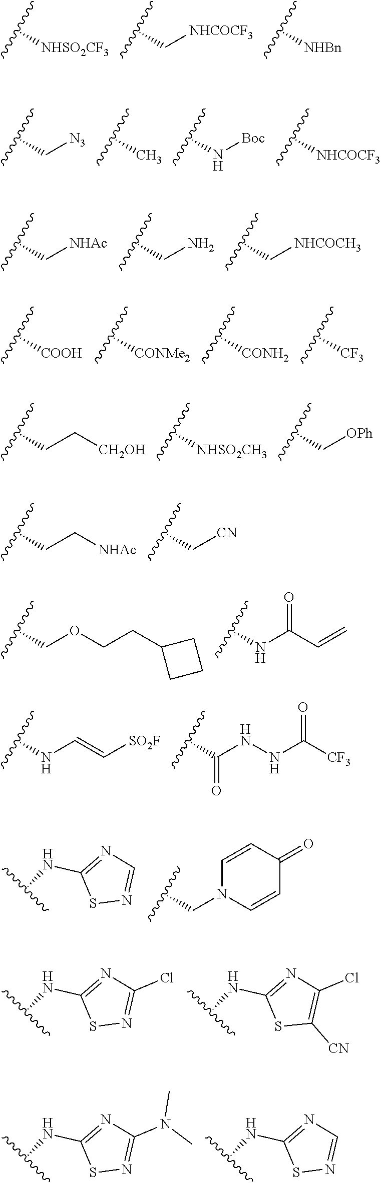

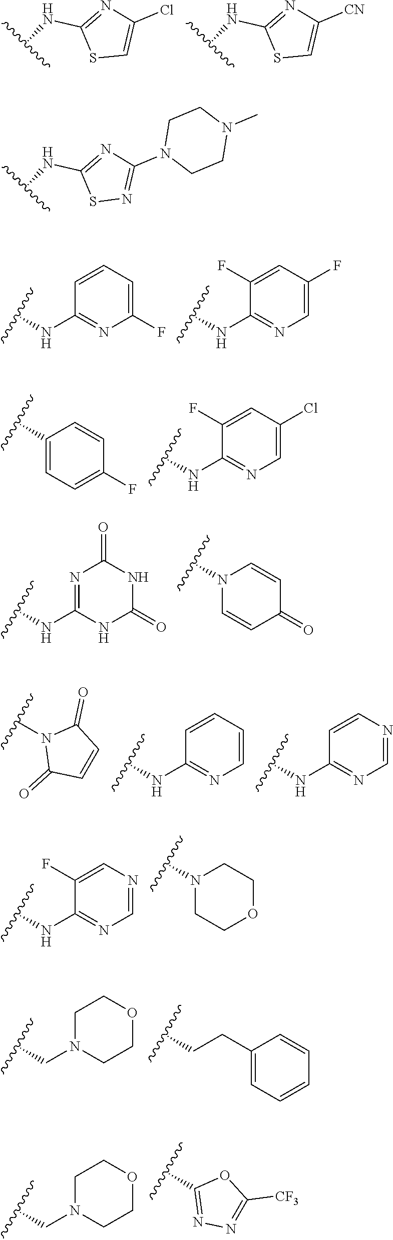

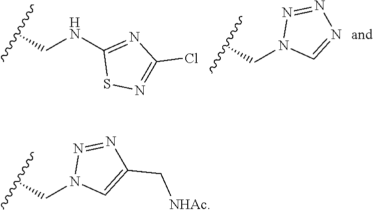

In certain embodiments, R²⁰⁰ is selected from

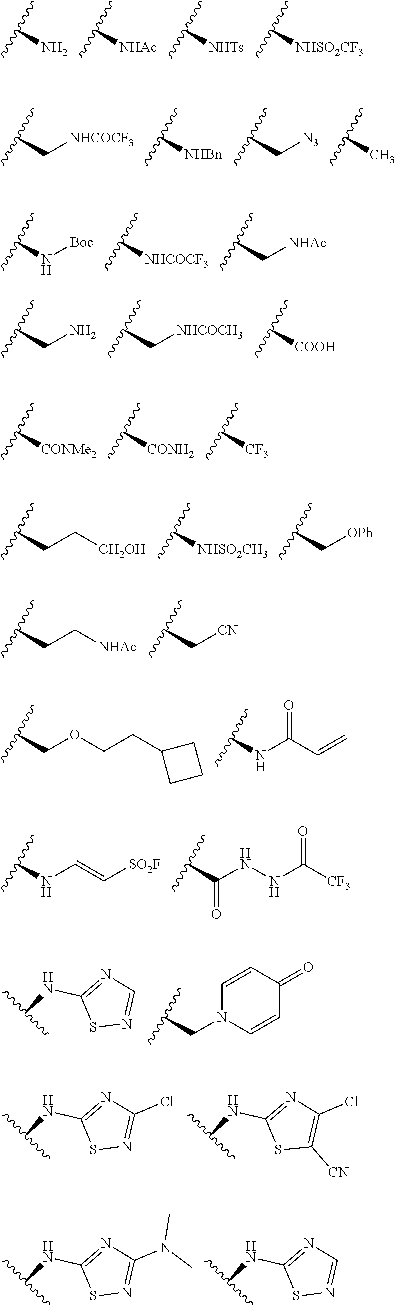

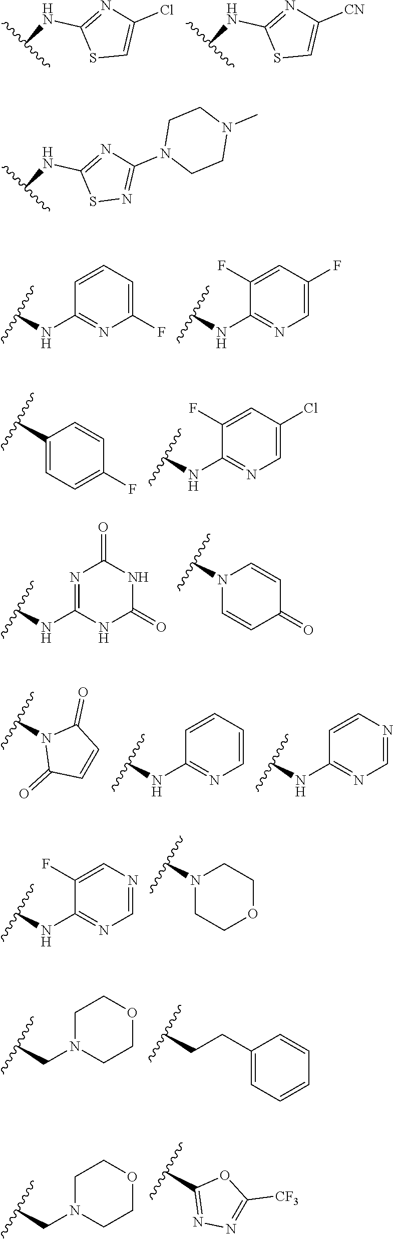

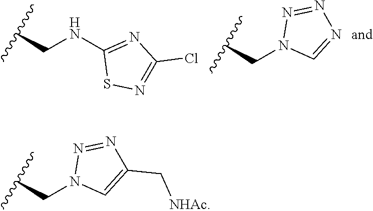

In certain embodiments R²⁰⁰ is selected from

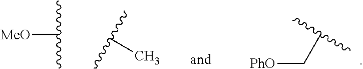

In certain embodiments R²⁰⁰ is selected from

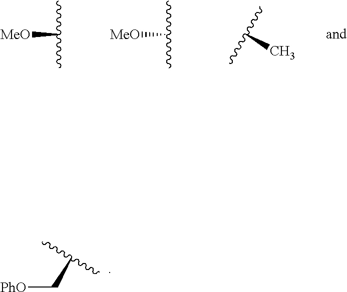

In certain embodiments R²⁰⁰ is selected from

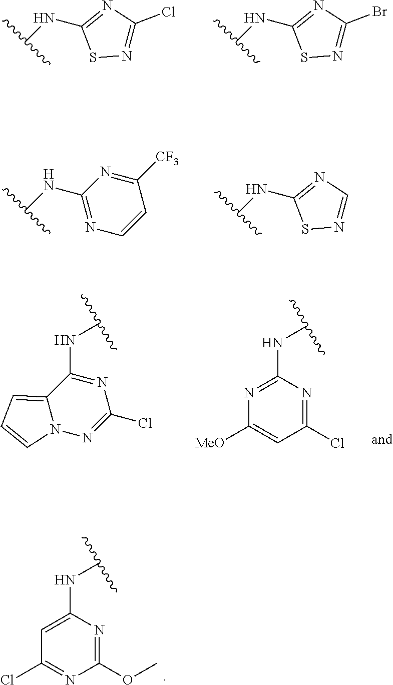

In certain embodiments R²⁰⁰ is selected from

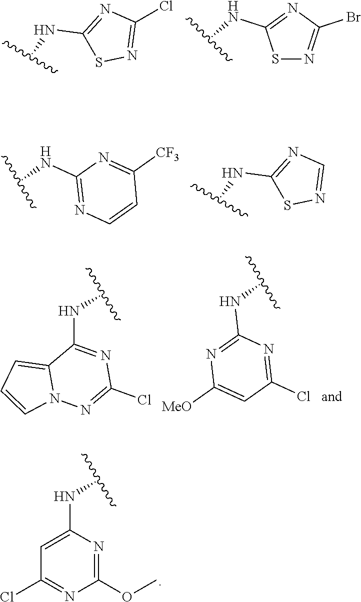

In certain embodiments R²⁰⁰ is selected from

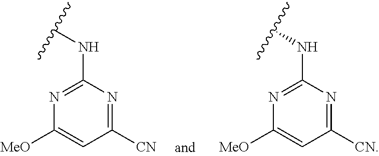

In certain embodiments R²⁰⁰ is selected from

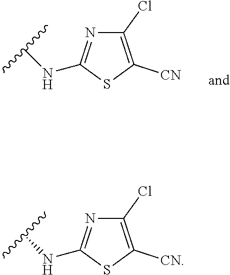

In certain embodiments R²⁰⁰ is selected from

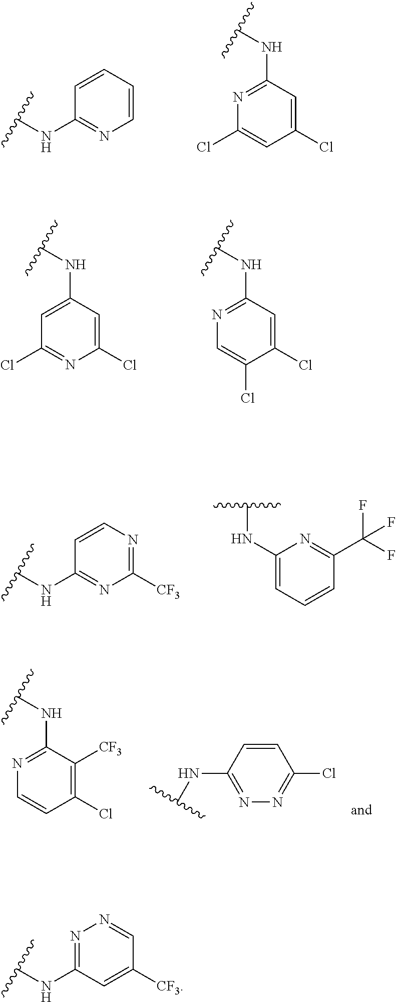

In certain embodiments R²⁰⁰ is selected from

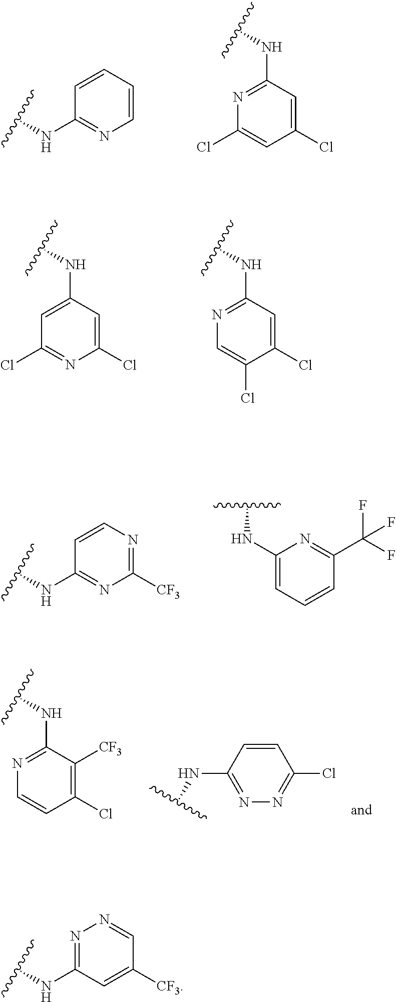

In certain embodiments R²⁰⁰ is selected from

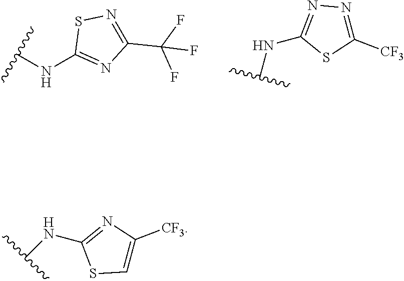 [SMILES: *Nc1nc(C(F)(F)F)ns1 ; *Nc1nnc(C(F)(F)F)s1 ; *Nc1nc(C(F)(F)F)cs1]

In certain embodiments R²⁰⁰ is selected from

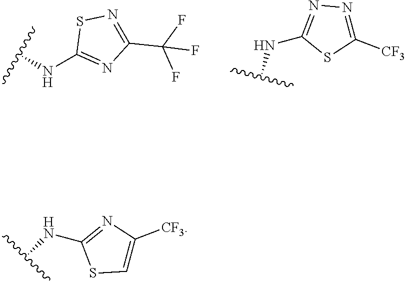

In certain embodiments R²⁰⁰ is selected from

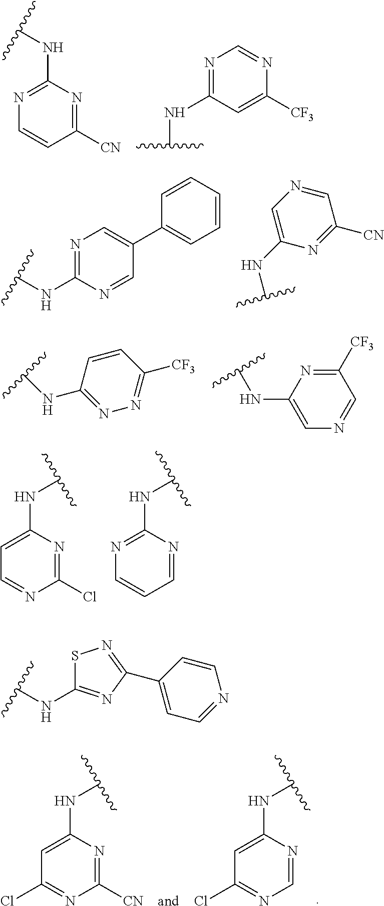

In certain embodiments R²⁰⁰ is selected from

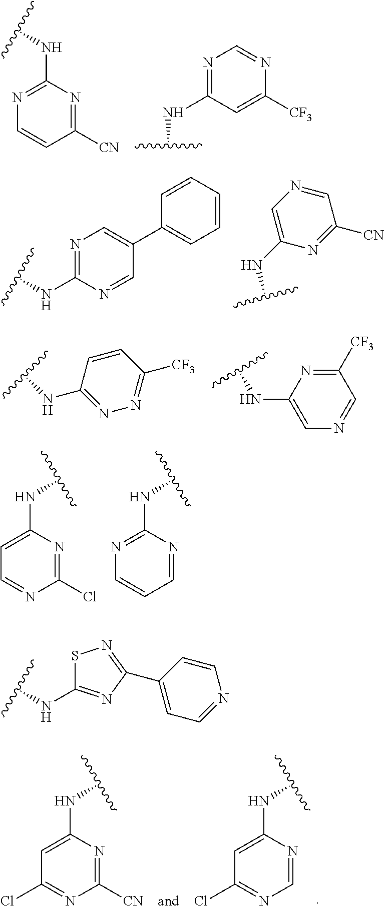

In certain embodiments R²⁰⁰ is selected from

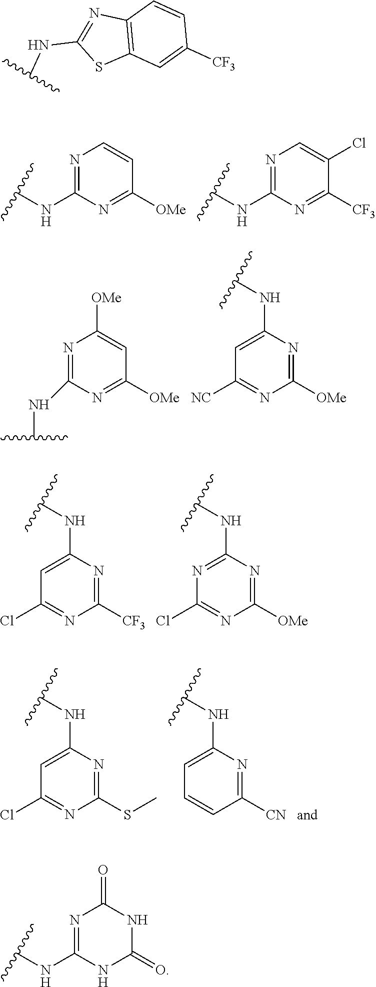

In certain embodiments R²⁰⁰ is selected from

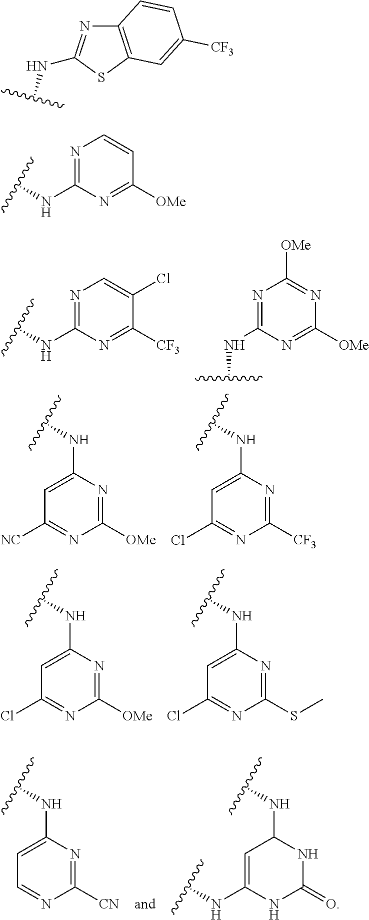

In certain embodiments R²⁰⁰ is selected from

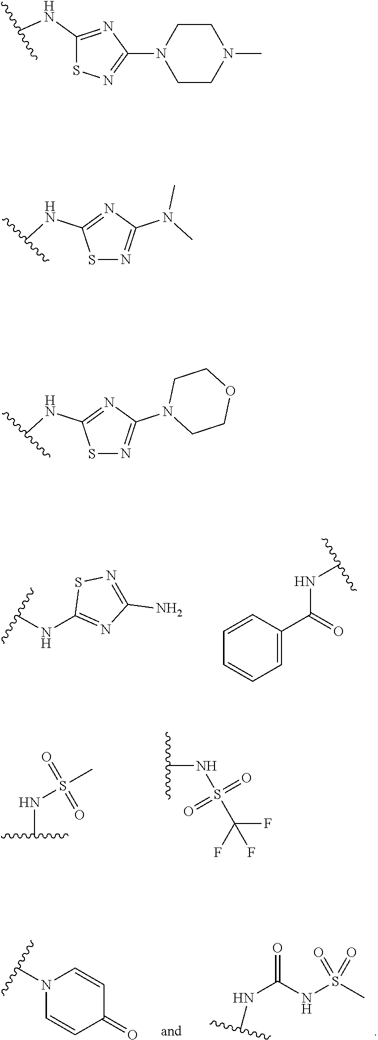

In certain embodiments R²⁰⁰ is selected from

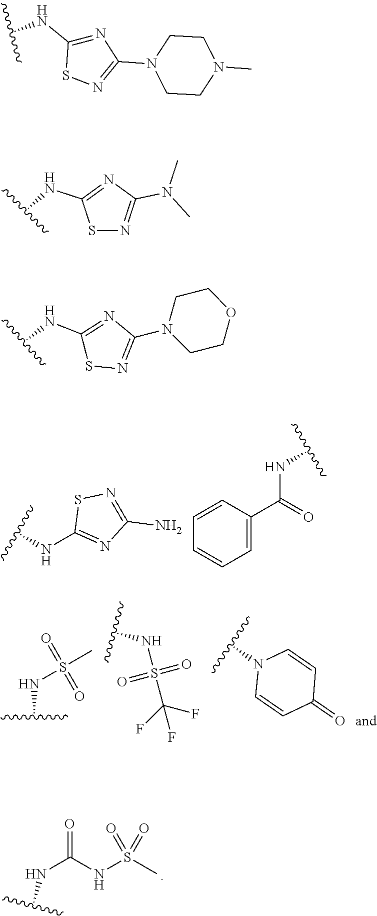

In certain embodiments R²⁰⁰ is selected from

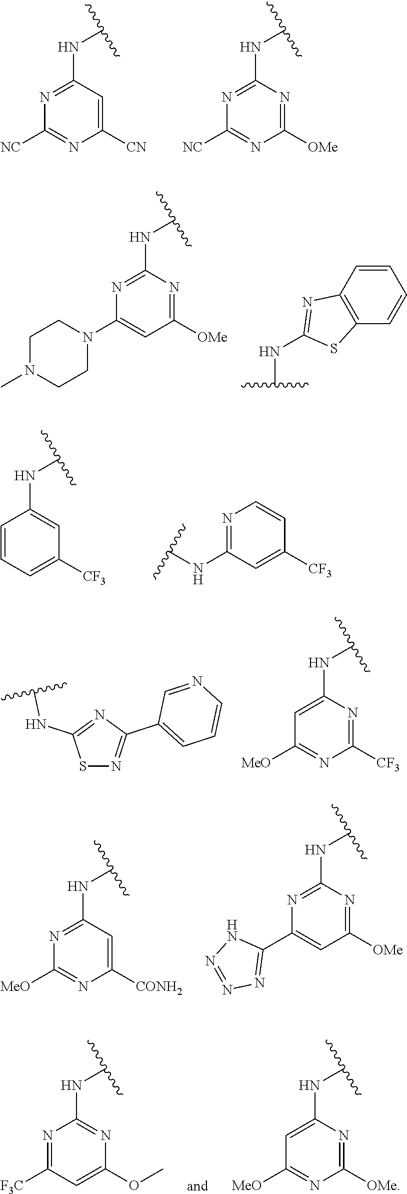

In certain embodiments R²⁰⁰ is selected from

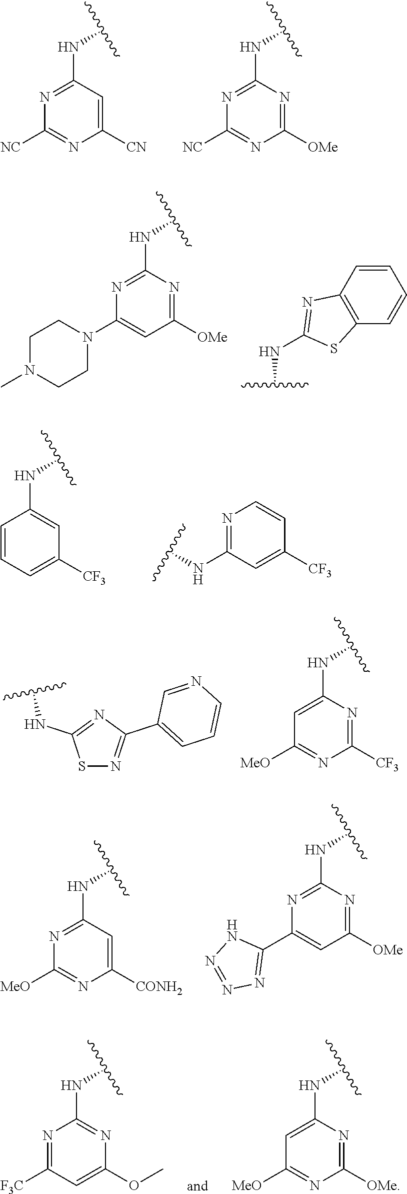

In certain embodiments R²⁰⁰ is selected from

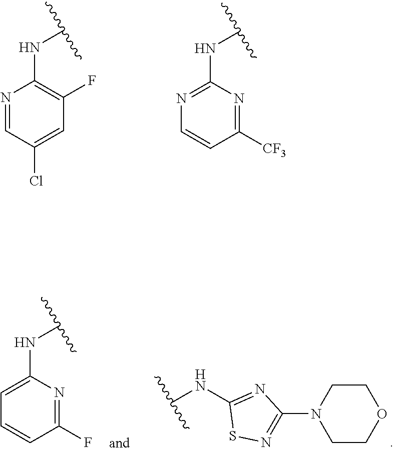 [SMILES: *Nc1ncc(Cl)cc1F ; *Nc1nccc(C(F)(F)F)n1 ; *Nc1cccc(F)n1 ; *Nc1nc(N2CCOCC2)ns1]

In certain embodiments R²⁰⁰ is selected from

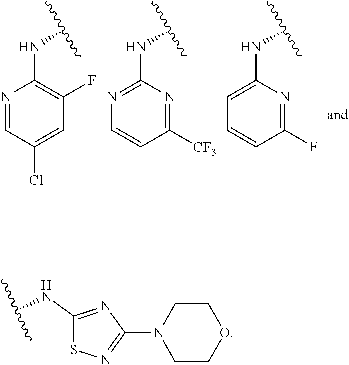

In certain embodiments R is selected from

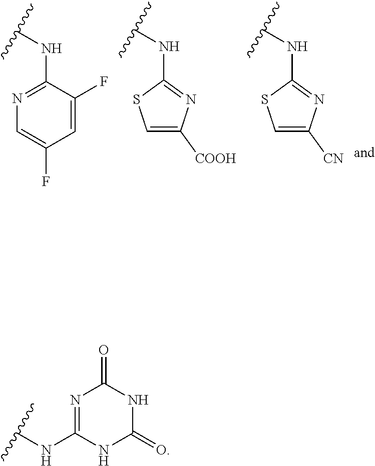

In certain embodiments R²⁰⁰ is selected from

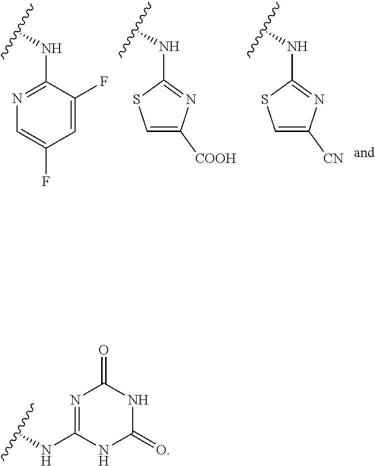

In certain embodiments R²⁰⁰ is selected from

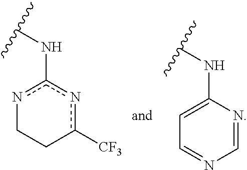

In certain embodiments R²⁰⁰ is selected from

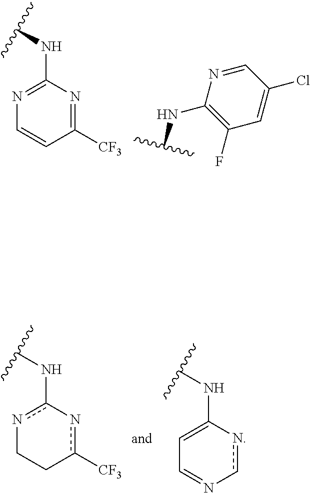

In certain embodiments R²⁰⁰ is selected from

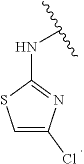 [SMILES: *Nc1nc(Cl)cs1]

In certain embodiments R²⁰⁰ is selected from

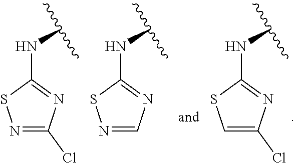

In certain embodiments R²⁰⁰ is selected from

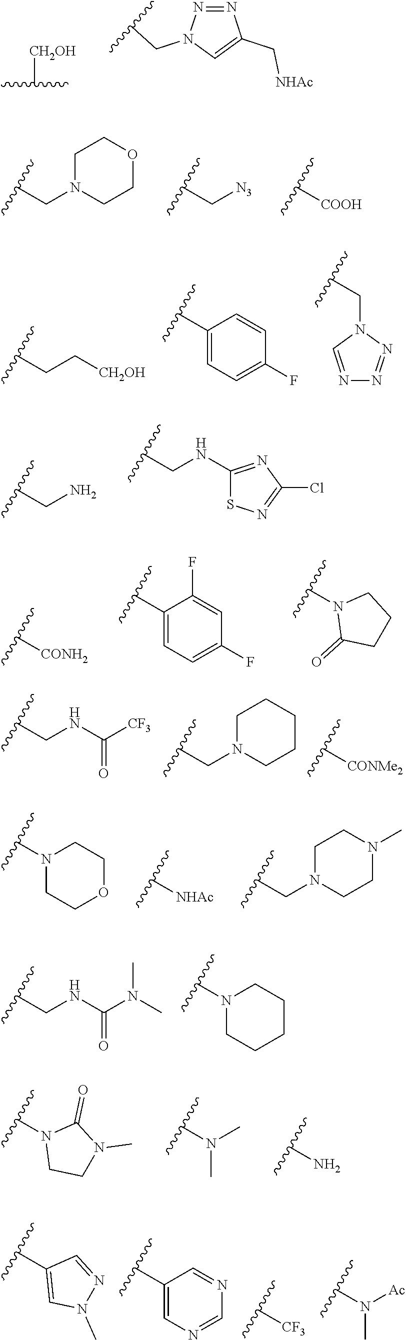

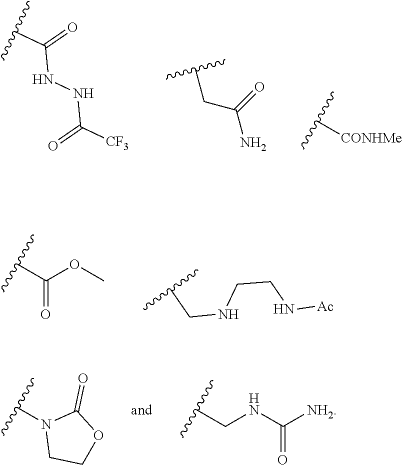

In certain embodiments R²⁰⁰ is selected from

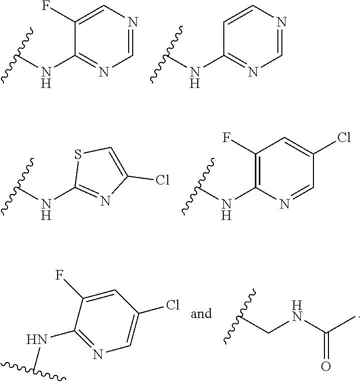

In certain embodiments R²⁰⁰ is selected from

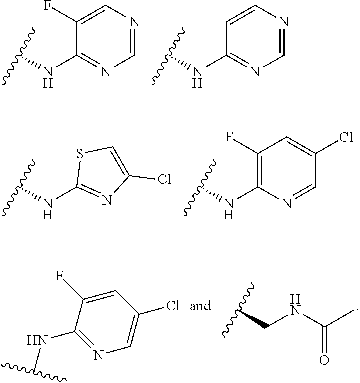

In certain embodiments R²⁰⁰ is a spirocyclic heterocycle for example

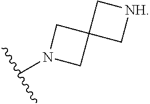 [SMILES: *N1CC2(CNC2)C1]

In certain embodiments R²⁰⁰ is a silicon containing heterocycle for example

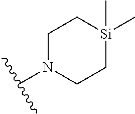 [SMILES: *N1CC[Si](C)(C)CC1]

In certain embodiments R²⁰⁰ is substituted with SF₅ for example

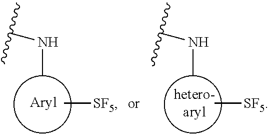

In certain embodiments R²⁰⁰ is substituted with a sulfoxime for example

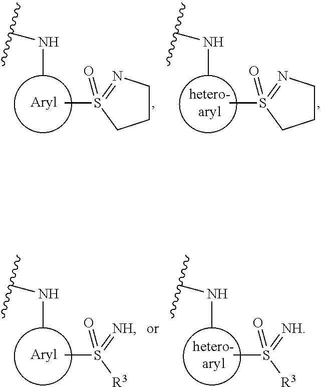

In certain embodiments R²⁰⁰ is

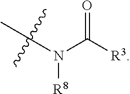

In certain embodiments R²⁰⁰ is

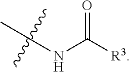

In certain embodiments R²⁰⁰ is

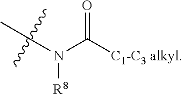

In certain embodiments R²⁰⁰ is

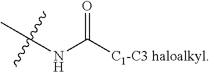

In certain embodiments R²⁰⁰ is

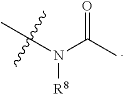

In certain embodiments R²⁰⁰ is

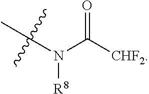

In certain embodiments R²⁰⁰ is

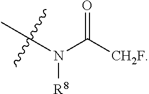

In certain embodiments R²⁰⁰ is

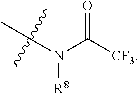

In certain embodiments R²⁰⁰ is

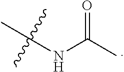 [SMILES: *NC(C)=O ; "."]

In certain embodiments R²⁰⁰ is

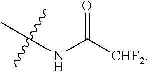 [SMILES: *NC(=O)C(F)F]

In certain embodiments R²⁰⁰ is

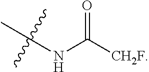

In certain embodiments R²⁰⁰ is

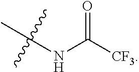 [SMILES: *NC(=O)C(F)(F)F]

### Embodiments of R¹⁰

In certain embodiments, R¹⁰ is selected from bicyclic heterocycle.

In certain embodiments, R¹⁰ is selected from spirocyclic heterocycle.

In certain embodiments, R¹⁰ is selected from —NR⁶-heterocycle.

In certain embodiments, R¹⁰ is selected from

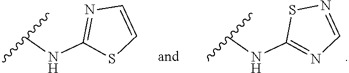 [SMILES: *Nc1nccs1 ; *Nc1ncns1]

In certain embodiments, R¹⁰ is selected from

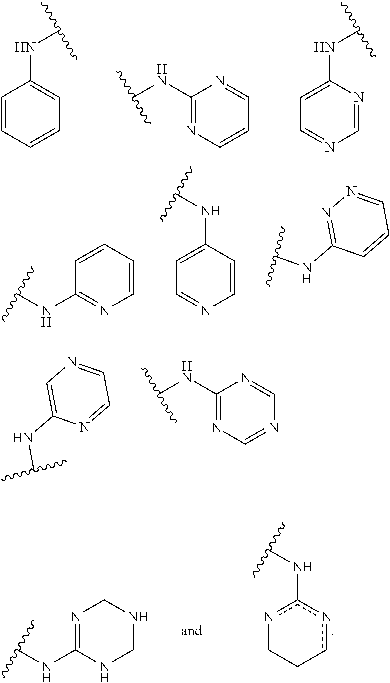

In certain embodiments, R¹⁰ is selected from

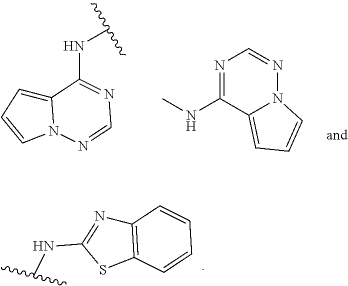

In certain embodiments, R¹⁰ is selected from

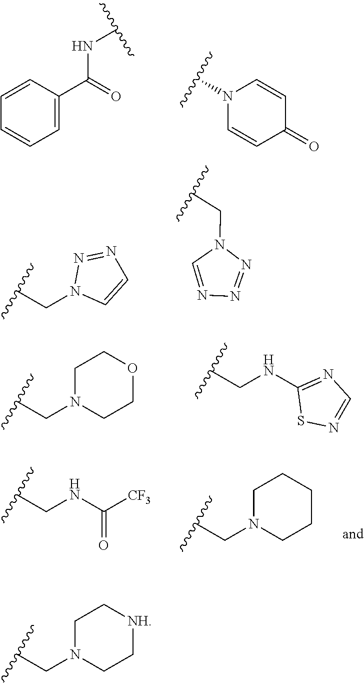

### Embodiments of Cycle

In certain embodiments Cycle is selected from

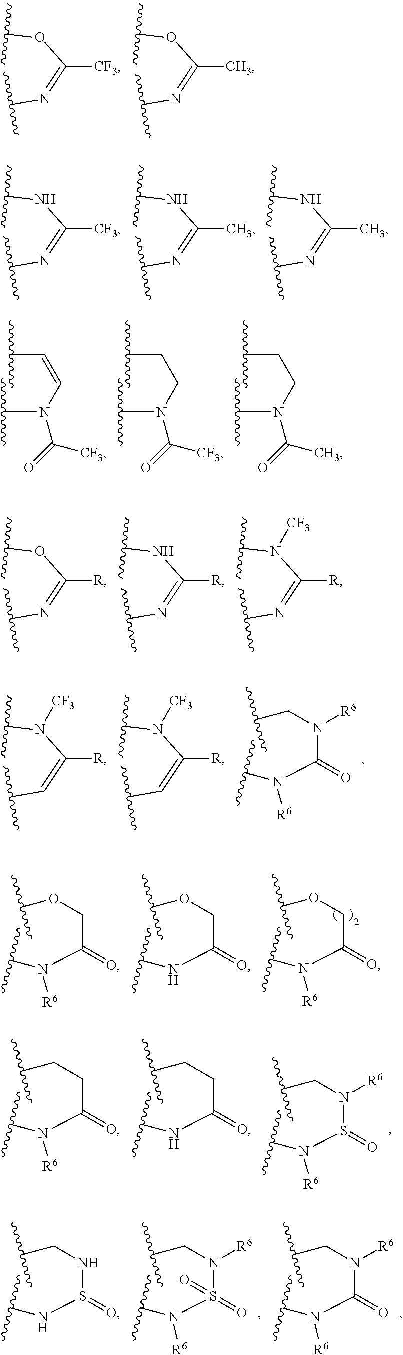

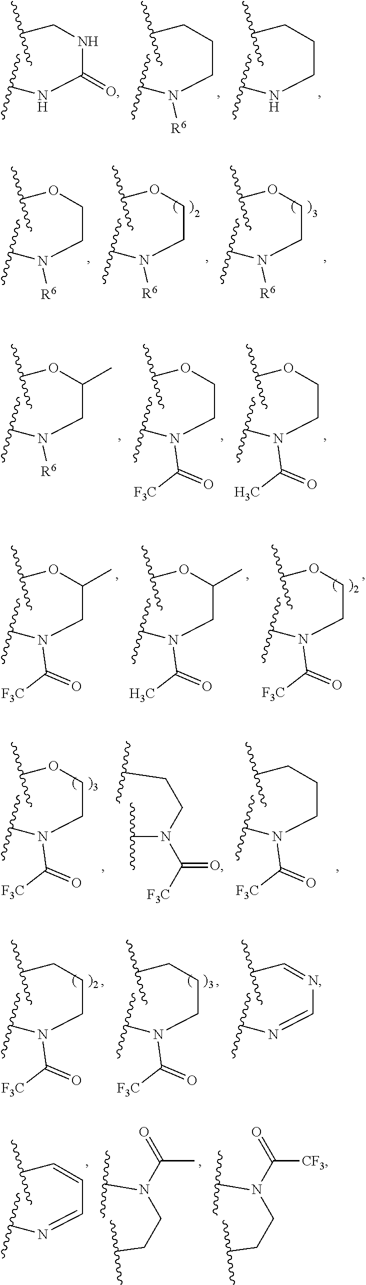

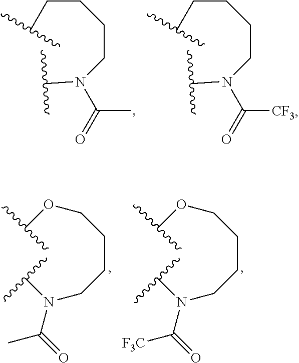

### Embodiments of R³⁰

In one embodiment R³⁰ is selected from:

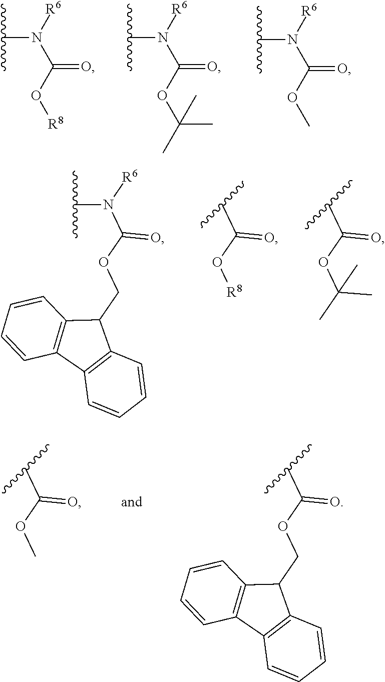

### Additional Embodiments of the Present Invention

1. In certain embodiments a compound is provided of Formula

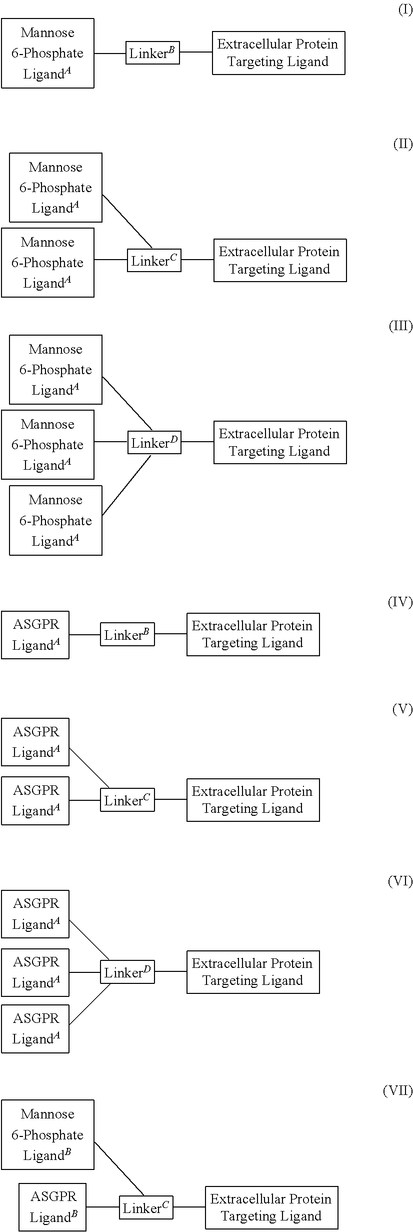

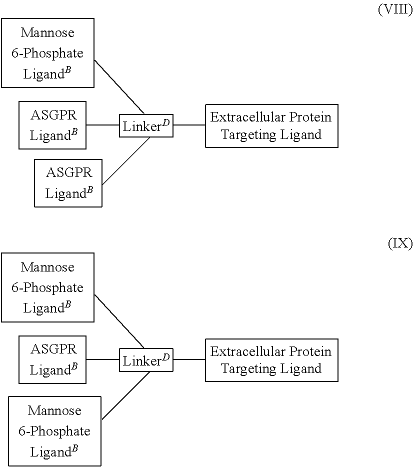

or a pharmaceutically acceptable salt thereof;

wherein:

Mannose 6-Phosphate Ligand^{A} is selected from:

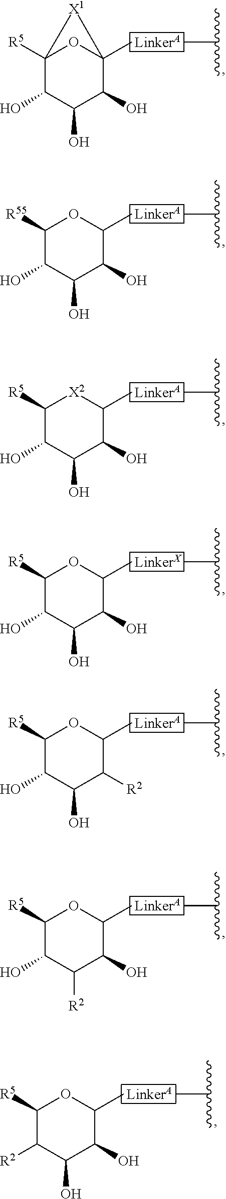

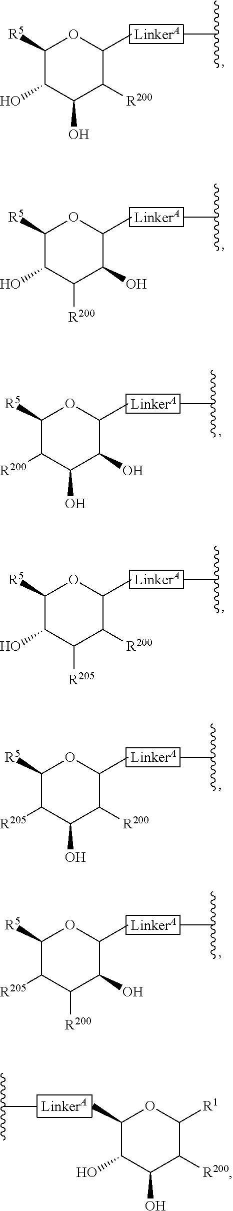

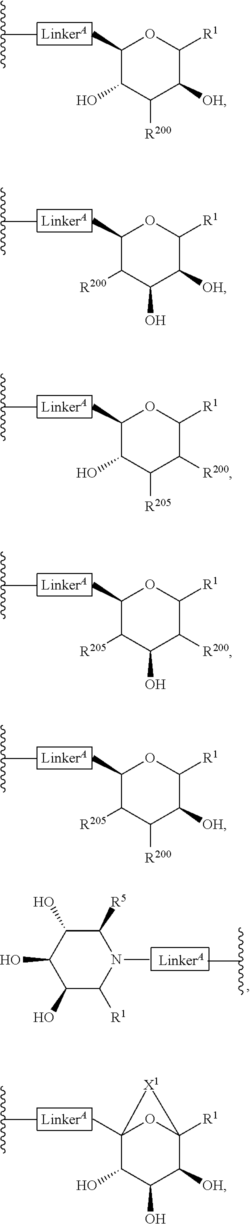

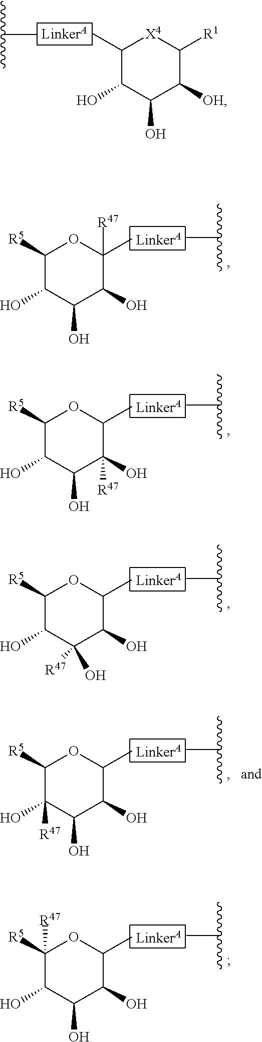

Mannose 6-Phosphate Ligand^{B} is selected from:
(i) Mannose 6-Phosphate Ligand^{A}; and

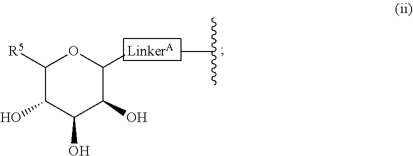

ASGPR Ligand^{A} is selected from:

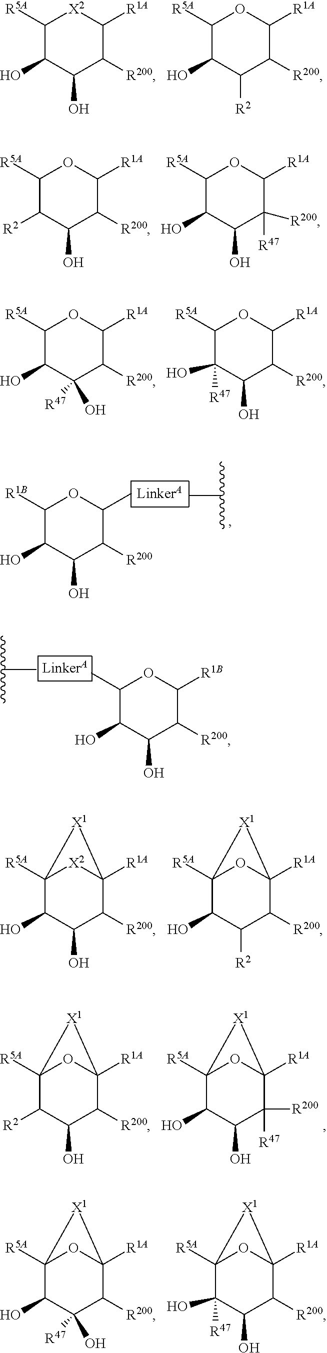

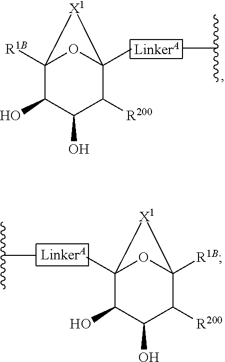

ASGPR Ligand^{B} is selected from:
(iv) ASGPR Ligand^{A};

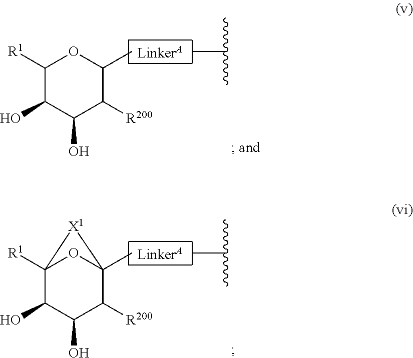

X¹ is 1 to 5 contiguous atoms independently selected from O, S, S(O), S(O)₂, N(R⁶), and C(R⁴)(R⁴), wherein if X¹ is 1 atom then X¹ is O, S, S(O), S(O)₂, N(R⁶), or C(R⁴)(R⁴), if X¹ is 2 atoms then no more than 1 atom of X¹ is O, S, or N(R⁶), if X¹ is 3, 4, or 5 atoms then no more than 2 atoms of X¹ are O, S, or N(R⁶);
X² is NR⁸, S, NC(O)R³, or CR⁴R⁸,
X³ is bond, NR⁸, NC(O)R³, S, or CR⁴R⁸,
X⁴ is NR⁸, NC(O)R³, S, or CR⁴R⁸,
R² is selected from F, SH, OR⁴³, and NHR⁸;
R²⁰⁰ and R²⁰⁵ are independently selected from hydrogen, halogen, —SH, —O-alkyl, —NR⁸—C(O)—R³, —NR⁶-alkyl, aryl, heterocycle, heteroaryl, NR⁸—S(O)₂—R³, —NR⁶-heteroalkyl, —NR⁸—S(O)—R³, —NR⁸—C(S)—R³, —NR⁸—S(O)(NR⁶)—R³, —N═S(O)(R³)₂, —NR⁸C(O)NR⁹S(O)₂R³, —NR⁸—S(O)₂—R¹⁰, —NR⁸—C(NR⁶)—R³, R¹⁰, alkyl-C(O)—R³, —C(O)—R³, alkyl, haloalkyl, —OC(O)R³, and —NR⁸—C(O)R¹⁰ each of which is optionally substituted with 1, 2, 3, or 4 substituents;
R¹⁰ is selected from alkenyl, allyl, alkynyl, —NR⁶-alkenyl, —O-alkenyl, —NR⁶-alkynyl, —NR⁶-heteroaryl, —NR⁶-aryl, —O-heteroaryl, —O-aryl, —O-alkynyl, aryl, alkyl-NR⁸—C(O)—R³, alkyl-aryl, alkyl-heteroaryl with 1, 2, or 4 heteroatoms, alkyl-cyano, alkyl-OR⁶, alkyl-NR⁶R⁸, NR⁸—NR⁶—C(O)R³, NR⁸—S(O)₂—R³, each of which R¹⁰ is optionally substituted with 1, 2, 3, or 4 substituents;
R¹ is selected from hydrogen, heteroalkyl, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-P(O)(R³)₂, C₀-C₆alkyl-OP(O)(R³)₂, C₀-C₆alkyl-NR⁸P(O)(R³)₂, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, 3, or 4 substituents;
R^{1A} and R^{5A} are independently selected from

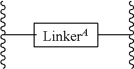

and R¹, wherein 1 of R^{1A} and R^{5A} is

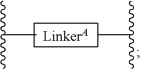

R^{1B} is selected from 4-membered heterocycle, 7-membered heterocycle, haloalkyl with 4, 5, or 6 halogen atoms, spiroheterocycle, and spirocycloalkyl;
R⁵ is selected from

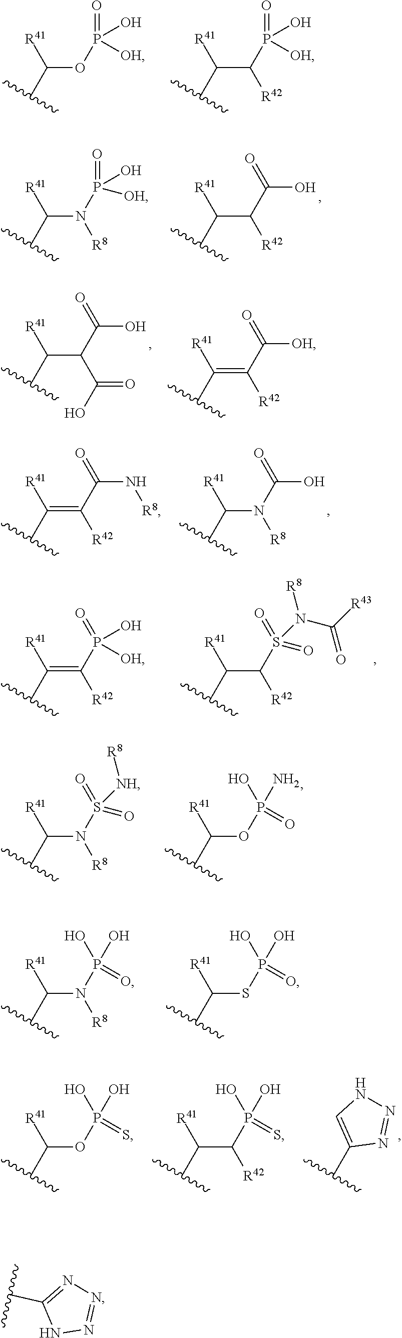

heteroaryl, heterocycle, and R⁵⁵;
R⁵⁵ is selected from

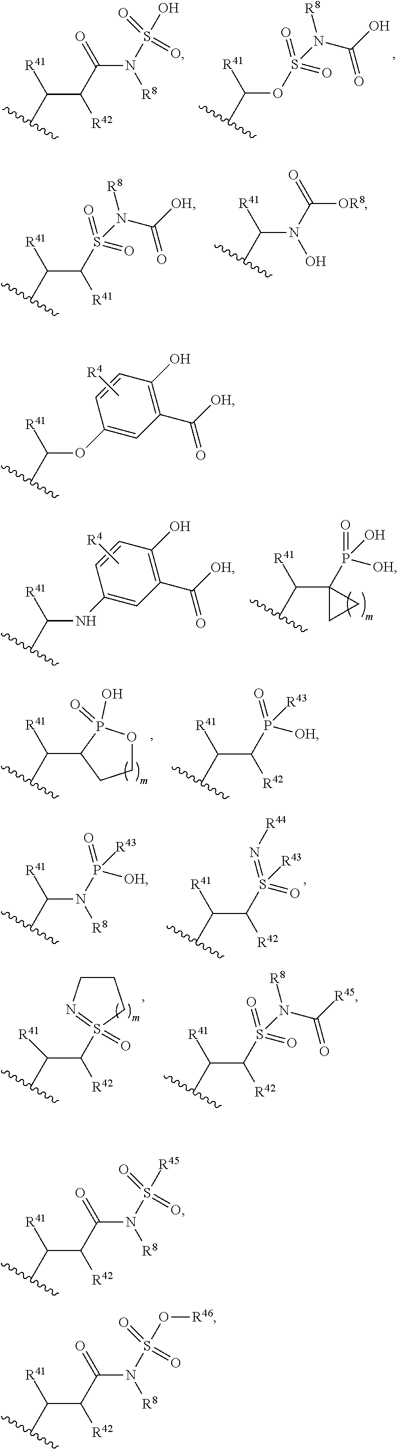

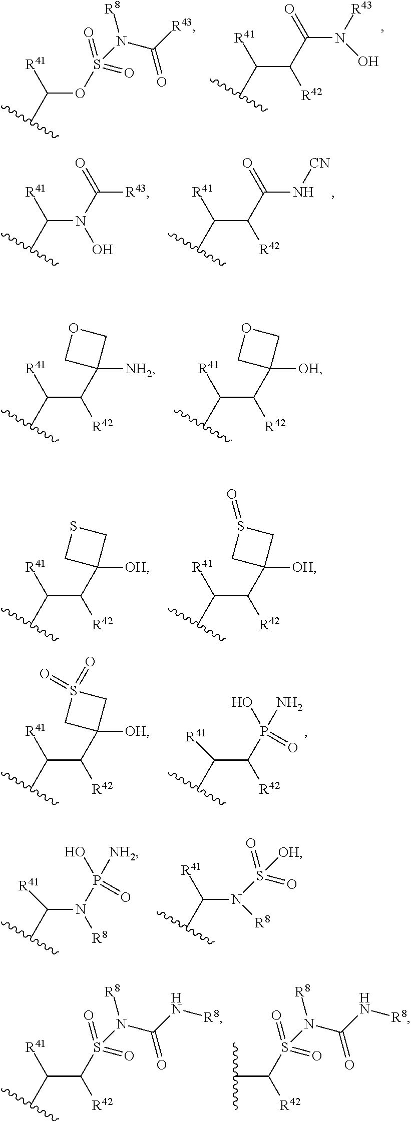

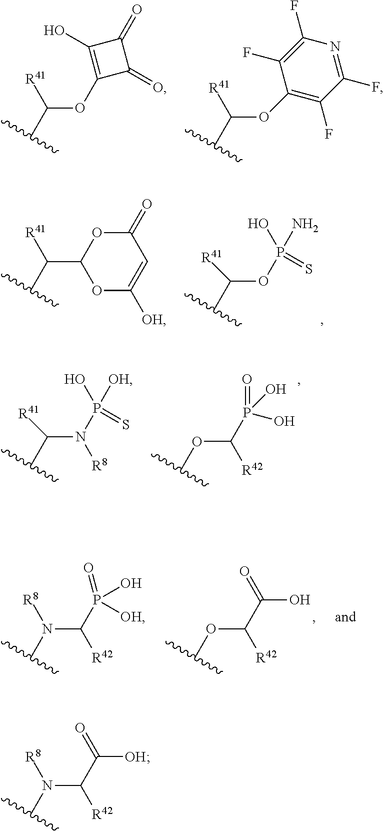

m is 1, 2, or 3;
R⁴¹ and R⁴² are independently selected from hydrogen, heteroalkyl, alkyl, arylalkyl, heteroarylalkyl, alkenyl, alkynyl, aryl, heteroaryl, and heterocycle;
R⁴³ is selected from alkyl, alkenyl, alkynyl, aryl, -alkyl-aryl, heteroaryl, -alkyl-heteroaryl, heterocycle, and -alkyl-heterocycle, each of which is optionally substituted with 1, 2, 3, or 4 substituents;
R⁴⁴ is selected from hydrogen, alkyl, cyano, alkenyl, alkynyl, aryl, heteroaryl, and heterocycle each of which except hydrogen and cyano is optionally substituted with 1, 2, 3, or 4 substituents;
R⁴⁵ is selected from cycloalkyl, aryl, heteroaryl, and heterocycle each of which is optionally substituted with 1, 2, 3, or 4 substituents;
R⁴⁶ is selected from hydrogen, alkyl, alkenyl, alkynyl, aryl, heteroaryl, and heterocycle each of which except hydrogen is optionally substituted with 1, 2, 3, or 4 substituents;
R⁴⁷ is selected from C₁-C₃ alkyl and C₁-C₃ haloalkyl;
R³ at each occurrence is independently selected from hydrogen, alkyl, heteroalkyl, haloalkyl, arylalkyl, heteroarylalkyl, alkenyl, alkynyl, aryl, heteroaryl, heterocycle, —OR⁸, and —NR⁸R⁹;
R⁴ is independently selected at each occurrence from hydrogen, halogen, heteroalkyl, alkyl, haloalkyl, arylalkyl, heteroarylalkyl, alkenyl, alkynyl, aryl, heteroaryl, heterocycle, —OR⁶, —NR⁶R⁷, C(O)R³, S(O)R³, C(S)R³, and S(O)₂R³;
R⁶ and R⁷ are independently selected at each occurrence from hydrogen, heteroalkyl, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, heteroalkyl, alkyl, arylalkyl, heteroarylalkyl, alkenyl, alkynyl, aryl, heteroaryl, and heterocycle;
each Linker^{X} is selected from Linker^{Y} and Linker^{Z};
each Linker^{Y} is selected from:

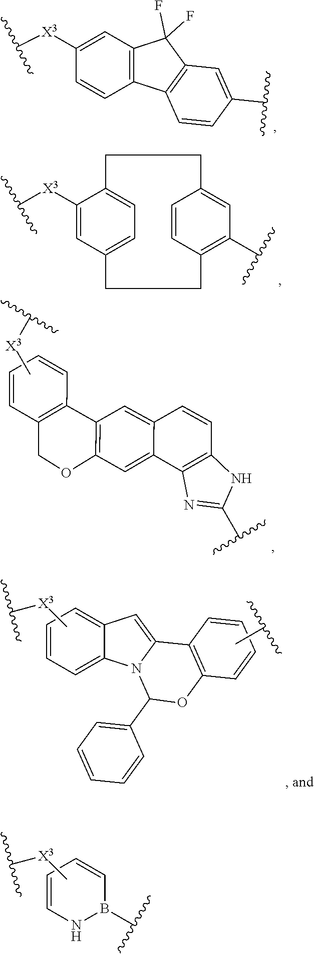

each of which is optionally substituted with 1, 2, 3, or 4 substituents;
each Linker^{Z} is selected from bivalent heteroaryl, aryl, heterocycle, cycloalkyl, —NR⁸C(O)-aryl, and —NR⁸C(O)-heteroaryl each of which is optionally substituted with 1, 2, 3, or 4 substituents;
each Linker^{A} is a bond or a moiety that covalently links the sugar (e.g. the C1 or C5 position) to Linker^{B};
Linker^{B} is a bond or a moiety that covalently links Linker^{A} to an Extracellular Protein Targeting Ligand;
Linker^{C} is a moiety that covalently links Linker^{A} to an Extracellular Protein Targeting Ligand;
Linker^{D} is a moiety that covalently links Linker^{A} to an Extracellular Protein Targeting Ligand;
Extracellular Protein Targeting Ligand is a chemical moiety that binds to the targeted disease-mediating extracellular protein; and
when a compounds is “optionally substituted” it may be substituted as allowed by valence with one or more groups selected from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, heteroalkyl, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, —SF₅,

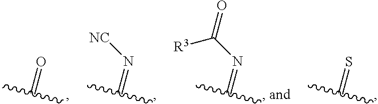

wherein the optional substituent is selected such that a stable compound results.

2. The compound of embodiment 1 wherein the compound is of Formula:

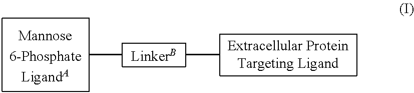

or a pharmaceutically acceptable salt thereof.

3. The compound of embodiment 1 wherein the compound is of Formula:

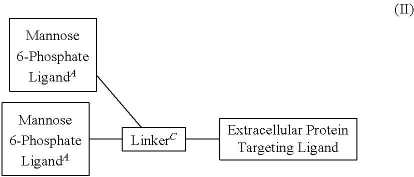

or a pharmaceutically acceptable salt thereof.

4. The compound of embodiment 1 wherein the compound is of Formula:

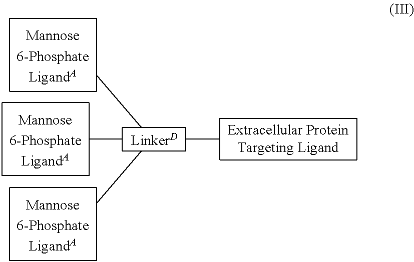

or a pharmaceutically acceptable salt thereof.

5. The compound of embodiment 1 wherein the compound is of Formula:

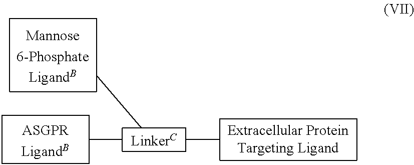

or a pharmaceutically acceptable salt thereof.

6. The compound of embodiment 1 wherein the compound is of Formula:

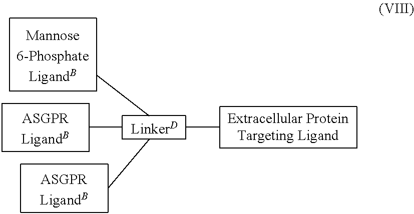

or a pharmaceutically acceptable salt thereof.

7. The compound of embodiment 1 wherein the compound is of Formula:

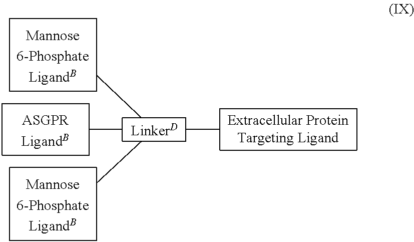

or a pharmaceutically acceptable salt thereof.

8. The compound of any one of embodiments 1-7 wherein Mannose 6-Phosphate Ligand^{A} is selected from:

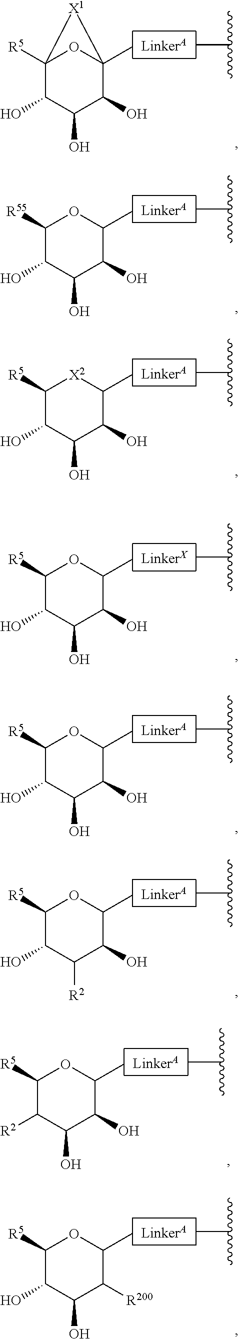

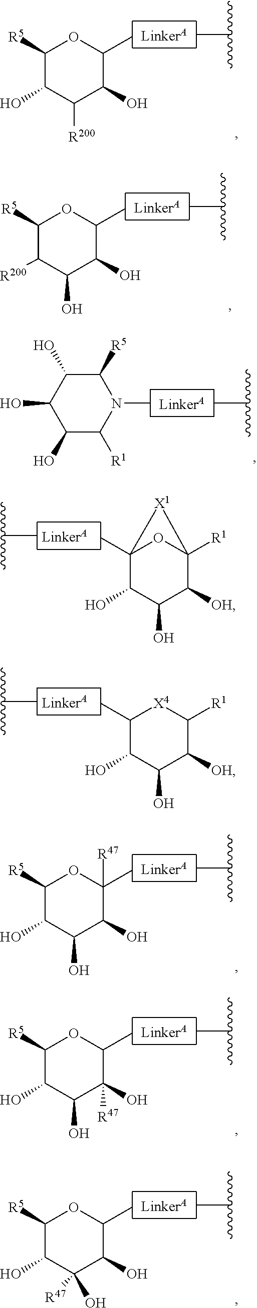

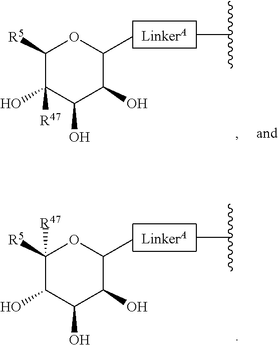

9. The compound of any one of embodiments 1-8 wherein
R⁴³ is selected from alkyl, alkenyl, alkynyl, aryl, heteroaryl, and heterocycle each of which is optionally substituted with 1, 2, 3, or 4 substituents;

10. The compound of any one of embodiments 1-9 wherein
R⁵⁵ is selected from

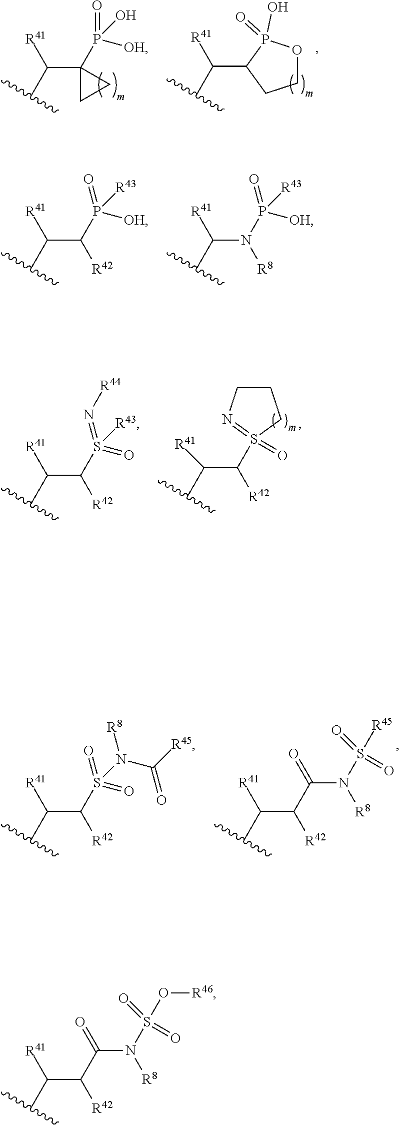

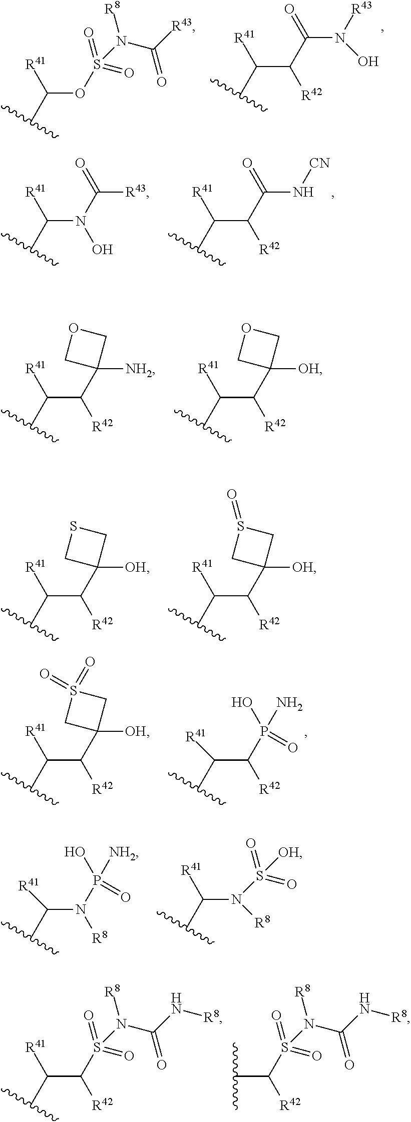

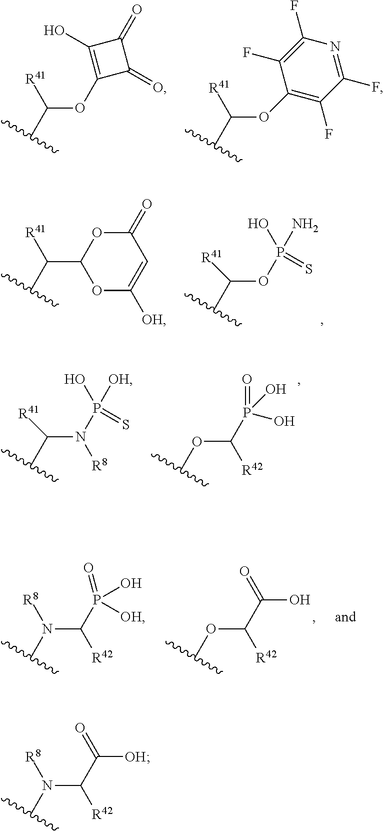

11. The compound of embodiment 1 wherein the compound is of Formula:

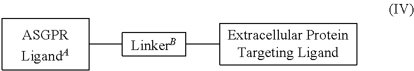

or a pharmaceutically acceptable salt thereof.

12. The compound of embodiment 1 wherein the compound is of Formula:

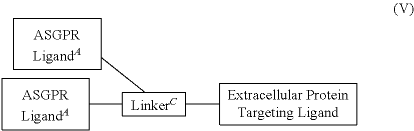

or a pharmaceutically acceptable salt thereof.

13. The compound of embodiment 1 wherein the compound is of Formula:

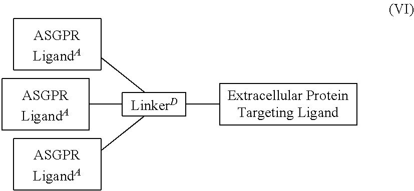

or a pharmaceutically acceptable salt thereof.

14. The compound of any one of embodiments 1-13 wherein
X² is NR⁸, S, NC(O)R³, or CR⁴R⁸,
X³ is bond, NR⁸, NC(O)R³, S, or CR⁴R⁸, and
X⁴ is NR⁸, NC(O)R³, S, or CR⁴R⁸.

15. The compound of any one of embodiments 1-14 wherein
R²⁰⁰ and R²⁰⁵ are independently selected from hydrogen, —NR⁸—C(O)—R³, —NR⁶-alkyl, aryl, heterocycle, heteroaryl, NR⁸—S(O)₂—R³, —NR⁶-heteroalkyl, —NR⁸—S(O)—R³, —NR⁸—C(S)—R³, —NR⁸—S(O)(NR⁶)—R³, —N═S(O)(R³)₂, —NR⁸C(O)NR⁹S(O)₂R³, —NR⁸—S(O)₂—R¹⁰, —NR⁸—C(NR⁶)—R³, R¹⁰, alkyl-C(O)—R³, —C(O)—R³, alkyl, haloalkyl, —OC(O)R³, and —NR⁸—C(O)R¹⁰ each of which is optionally substituted with 1, 2, 3, or 4 substituents; and
R⁴ is independently selected at each occurrence from hydrogen, heteroalkyl, alkyl, haloalkyl, arylalkyl, heteroarylalkyl, alkenyl, alkynyl, aryl, heteroaryl, heterocycle, —OR⁶, —NR⁶R⁷, C(O)R³, S(O)R³, C(S)R³, and S(O)₂R³.

16. The compound of any one of embodiments 1-15 wherein the ASGPR Ligand is

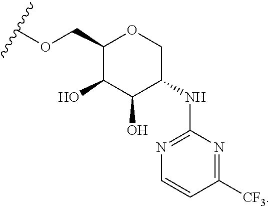 [SMILES: *OC[C@H]1OC[C@H](Nc2nccc(C(F)(F)F)n2)[C@@H](O)[C@H]1O]

17. The compound of any one of embodiments 1-15 wherein the ASGPR Ligand is

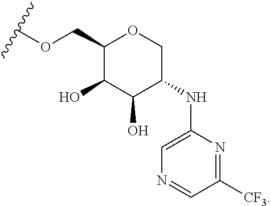 [SMILES: *OC[C@H]1OC[C@H](Nc2cncc(C(F)(F)F)n2)[C@@H](O)[C@H]1O]

18. The compound of any one of embodiments 1 to 17, wherein R¹ is selected from hydrogen, alkyl, F, Cl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, and C₀-C₆alkyl-S(O)₂R³ each of which is optionally substituted with 1, 2, 3, or 4 substituents.

19. The compound of any one of embodiments 1 to 17, wherein R¹ is selected from hydrogen, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, and C₀-C₆alkyl-S(O)₂R³ each of which is optionally substituted with 1, 2, or 3 substituents.

20. The compound of any one of embodiments 1 to 17, wherein R¹ is selected from hydrogen, C₀-C₆alkyl-OR⁶, and C₀-C₆alkyl-NR⁶R⁷.

21. The compound of any one of embodiments 1 to 20, wherein R³ is selected from aryl, heteroaryl, and heterocycle.

22. The compound of any one of embodiments 1 to 20, wherein R³ is —OR⁸.

23. The compound of any one of embodiments 1 to 20, wherein R³ is —NR⁸R⁹.

24. The compound of any one of embodiments 1 to 23, wherein R⁶ and R⁷ are independently selected at each occurrence from hydrogen, alkyl, aryl, haloalkyl, heteroaryl, heterocycle, C(O)R³, S(O)R³, C(S)R³, and S(O)₂R³.

25. The compound of any one of embodiments 1 to 23, wherein R⁶ and R⁷ are independently selected at each occurrence from hydrogen, alkyl, and C(O)R³, S(O)R³, C(S)R³, and S(O)₂R³.

26. The compound of any one of embodiments 1 to 25, wherein at least one of R⁶ and R⁷ are hydrogen.

27. The compound of any one of embodiments 1 to 26, wherein R⁸ and R⁹ are independently selected at each occurrence from hydrogen, alkyl, aryl, heteroaryl, and heterocycle.

28. The compound of any one of embodiments 1 to 26, wherein R⁸ and R⁹ are independently selected at each occurrence from hydrogen and alkyl.

29. The compound of any one of embodiments 1 to 26, wherein R⁸ and R⁹ are both hydrogen at each occurrence.

30. The compound of any one of embodiments 1 to 29, wherein Linker^{A} is selected from:

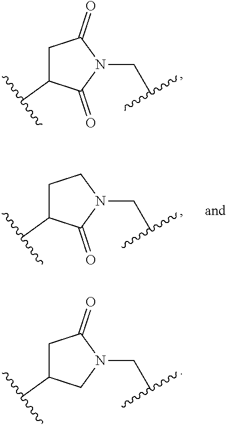

31. The compound of any one of embodiments 1 to 29, wherein Linker^{A} is selected from:

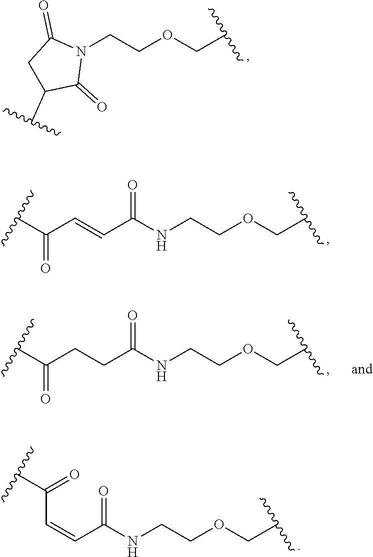 [SMILES: *COCCN1C(=O)CC(*)C1=O ; *COCCNC(=O)/C=C/C(*)=O ; *COCCNC(=O)CCC(*)=O ; *COCCNC(=O)/C=C\C(*)=O]

32. The compound of any one of embodiments 1 to 29, wherein Linker^{A} is selected from:

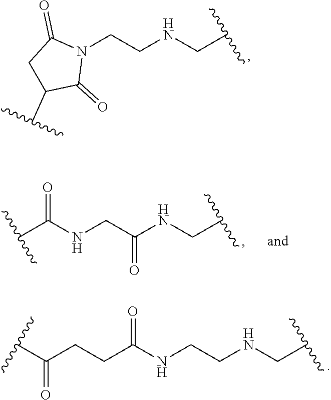

33. The compound of any one of embodiments 1, 2, 8-11, and 14-32, wherein Linker^{B} is:

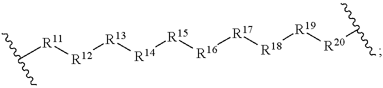

wherein:
R¹¹, R¹², R¹³, R¹⁴, R¹⁵, R¹⁶, R¹⁷, R⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[0-(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid, each of which is optionally substituted with 1, 2, 3, or 4 substituents independently selected from R²¹;
n is independently selected at each instance from 0, 1, 2, 3, 4, 5, 6, 7, 8, 9, or 10; and
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, aryl, heteroaryl, heterocyclyl, —SR³, —C(O)OR³,

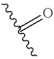 [SMILES: *=O]

34. The compound of embodiment 33, wherein R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, aryl, heteroaryl, and heterocycle.

35. The compound of embodiment 33 or 34, wherein 1, 2, 3, or 4 of R¹¹, R¹², R¹³, R¹⁴, R¹⁵, R¹⁶, R¹⁷, R¹⁸, and R¹⁹ is bond.

36. The compound of embodiment 33 or 34, wherein 1, 2, 3, or 4 of R¹¹, R¹², R¹³, R¹⁴, R¹⁵, R¹⁶, R¹⁷, R¹⁸, and R¹⁹ is an amino acid.

37. The compound of embodiment 33, wherein Linker^{B} is selected from:

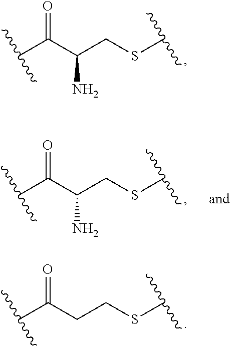 [SMILES: *SC[C@@H](N)C(*)=O ; *SC[C@H](N)C(*)=O ; *SCCC(*)=O]

38. The compound of embodiment 33, wherein Linker^{B} is selected from:

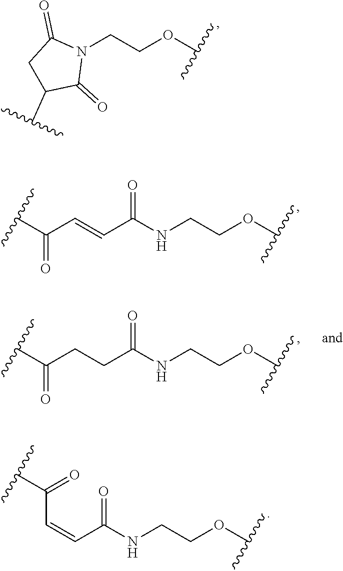 [SMILES: *OCCN1C(=O)CC(*)C1=O ; *OCCNC(=O)/C=C/C(*)=O ; *OCCNC(=O)CCC(*)=O ; *OCCNC(=O)/C=C\C(*)=O]

39. The compound of any one of embodiments 1, 3, 5, 8-10, 12 and 14-32, wherein Linker^{C} is selected from:

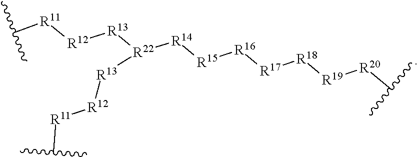

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid, each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, aryl, heteroaryl, heterocyclyl, —SR³, —C(O)OR³, —C(O)NR⁶NR⁷, —OR³,

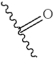 [SMILES: *=O]

and heterocycle; and
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹.

40. The compound of embodiment 39, wherein R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, aryl, heteroaryl, and heterocycle.

41. The compound of embodiment 39 or 40, wherein Linker^{C} is selected from:

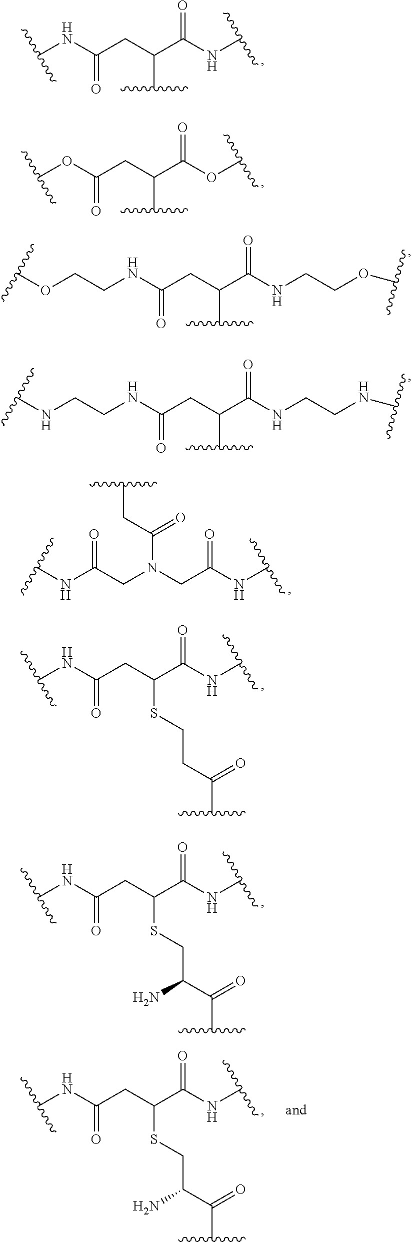

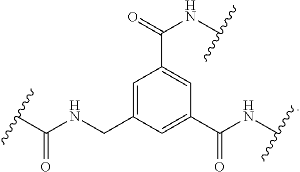 [SMILES: *NC(=O)c1cc(CNC(*)=O)cc(C(=O)N*)c1]

42. The compound of any one of embodiments 1, 4, 6-10, and 13-32, wherein Linker^{D} is selected from:

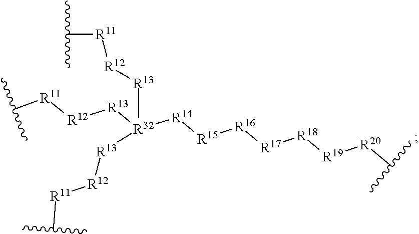

wherein:
R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹; and
X⁻ is an anionic group.

43. The compound of any one of embodiments 1 to 42, wherein the Extracellular Protein Targeting Ligand targets an immunoglobin.

44. The compound of any one of embodiments 1 to 42, wherein the Extracellular Protein Targeting Ligand targets IgA.

45. The compound of embodiment 44, wherein the Extracellular Protein Targeting Ligand is:

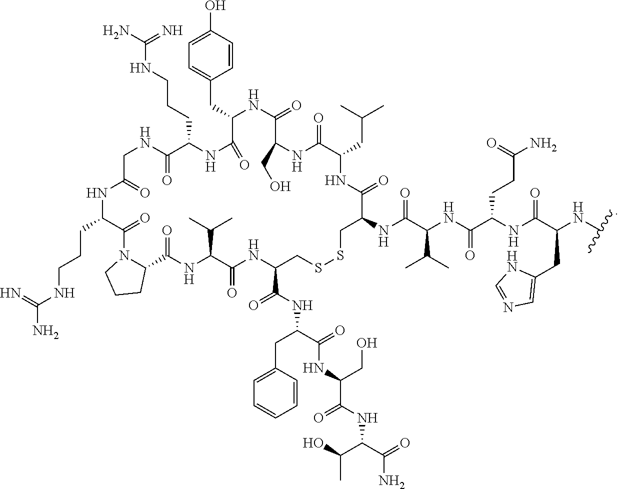 [SMILES: *N[C@@H](Cc1cnc[nH]1)C(=O)N[C@@H](CCC(N)=O)C(=O)N[C@H](C(=O)N[C@H]1CSSC[C@@H](C(=O)N[C@@H](Cc2ccccc2)C(=O)N[C@@H](CO)C(=O)N[C@H](C(N)=O)[C@@H](C)O)NC(=O)[C@H](C(C)C)NC(=O)[C@@H]2CCCN2C(=O)[C@H](CCCNC(=N)N)NC(=O)CNC(=O)[C@H](CCCNC(=N)N)NC(=O)[C@H](Cc2ccc(O)cc2)NC(=O)[C@H](CO)NC(=O)[C@H](CC(C)C)NC1=O)C(C)C]

46. The compound of any one of embodiments 1 to 42, wherein the Extracellular Protein Targeting Ligand targets IgG.

47. The compound of any one of embodiments 1 to 42, wherein the Extracellular Protein Targeting Ligand targets IgE.

48. The compound of any one of embodiments 1 to 42, wherein the Extracellular Protein Targeting Ligand targets TNF-α.

49. The compound of any one of embodiments 1 to 42, wherein the Extracellular Protein Targeting Ligand targets IL-1b.

50. The compound of any one of embodiments 1 to 42, wherein the Extracellular Protein Targeting Ligand targets IL-2.

51. The compound of any one of embodiments 1 to 42, wherein the Extracellular Protein Targeting Ligand targets IL-6.

52. The compound of any one of embodiments 1 to 42, wherein the Extracellular Protein Targeting Ligand targets IFN-7.

53. The compound of any one of embodiments 1 to 42, wherein the Extracellular Protein Targeting Ligand targets VEGF.

54. The compound of any one of embodiments 1 to 42, wherein the Extracellular Protein Targeting Ligand targets TGF-b1.

55. The compound of any one of embodiments 1 to 42, wherein the Extracellular Protein Targeting Ligand targets PCSK-9.

56. A pharmaceutical composition comprising a compound of any one of embodiments 1 to 55 and a pharmaceutically acceptable carrier.

57. A method of treating a disorder mediated by an effective amount of Extracellular Protein comprising administering a compound of any one of embodiments 1 to 55 that includes an Extracellular Protein Targeting Ligand that binds to the Extracellular Protein, or a pharmaceutically acceptable salt thereof, to a patient in need thereof.

58. The method of embodiment 57, wherein the extracellular protein is IgA and the disorder is selected from IgA nephropathy (Berger's disease), celiac disease, Crohn's disease, Henoch-Sconiein purpura (HSP), liner IgA bullous dermatosis, IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, α-chain disease, IgA monoclonal gammopathy, monoclonal gammopathy of undetermined significance (MGUS), and linear IgA bullous dermatosis.

59. The method of embodiment 57, wherein the extracellular protein is IgG and the disorder is selected from type 1 autoimmune pancreatitis, interstitial nephritis, Riedel's thyroiditis, storiform fibrosis, Mikulicz's disease, Kuttner's tumor, inflammatory pseudotumors, mediastinal fibrosis, retroperitoneal fibrosis (Ormond's disease), aortitis, periaortitis, proximal biliary strictures, idiopathic hypocomplementic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenic gravis, thyroid eye disease, chronic inflammatory demyelinating polyneuropathy, warm autoimmune hemolytic anemia, ankylosing spondylitis, primary Sjögren's syndrome, psoriatic arthritis, and systemic lupus erythematosus (SLE), sclerosing cholangitis, and IgG monoclonal gammopathy, monoclonal gammopathy of undetermined significance (MGUS).

60. The method of embodiment 57, wherein the extracellular protein is IgE and the disorder is selected from atopic asthma, allergic rhinitis, atopic dermatitis, IgE-mediated food allergy, IgE-mediated animal allergies, allergic conjunctivitis, allergic urticaria, anaphylactic shock, nasal polyposis, keratoconjunctivitis, mastocytosis, and eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, and monoclonal gammopathy of undetermined significance (MGUS).

61. The method of embodiment 57, wherein the disorder is dementia or Alzheimer's disease.

62. The method of embodiment 57, wherein the extracellular protein is TNF-α and the disorder is selected from rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia.

63. The method of embodiment 57, wherein the extracellular protein is IL-2 and the disorder is selected from host versus graft rejection in transplants and autoimmune disorders, including, but not limited to, multiple sclerosis, idiopathic arthritis, iritis, anterior uveitis, IL-2 induced hypotension, and psoriasis.

64. The method of embodiment 57, wherein the extracellular protein is IL-6 and the disorder is selected from Castleman's disease, metastatic castration-associated prostate cancer, renal cell carcinoma, large-cell lung carcinoma, ovarian cancer, rheumatoid arthritis, and asthma.

65. The method of embodiment 57, wherein the extracellular protein is IFN-7 and the disorder is selected from rheumatoid arthritis, multiple sclerosis (MS), corneal transplant rejection, and various autoimmune skin diseases such as psoriasis, alopecia areata, vitiligo, and acne vulgaris.

66. The method of embodiment 57, wherein the disorder is a cancer.

### V. Extracellular Protein Degrading Molecules with ASGPR and Mannose 6-Phosphate Receptor Ligands

In certain aspects a compound of the present invention has at least one ASGPR binding ligand and at least one mannose 6-phosphate receptor binding ligand.

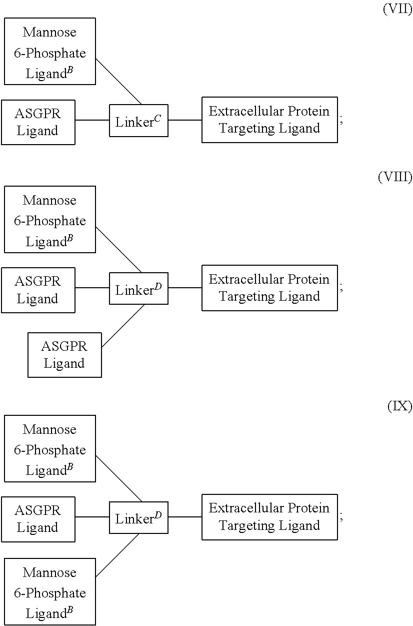

A compound of Formula VII, Formula VIII, or Formula IX may offer several advantages over traditional extracellular protein degrading compounds based on mannose 6-phosphate or galactose. For example, the compound of Formula VII, Formula VIII, or Formula IX may be more potent, require a smaller dose, have fewer side effects, provide faster degradation, provide more robust degradation, or target and degrade proteins that traditional extracellular protein degrading compounds cannot degrade.

In certain embodiments a compound of Formula VII, Formula VIII, or Formula IX degrades proteins by interacting with proteins of multiple tissues in the body. For example the compound may degrade proteins that are in circulation nearer to the liver by interacting with the ASGPR receptor and may degrade proteins circulating nearer to or in the brain by interacting with the mannose 6-phosphate receptor.

Non-limiting examples of compounds of Formula VII, Formula VIII, or Formula IX include:

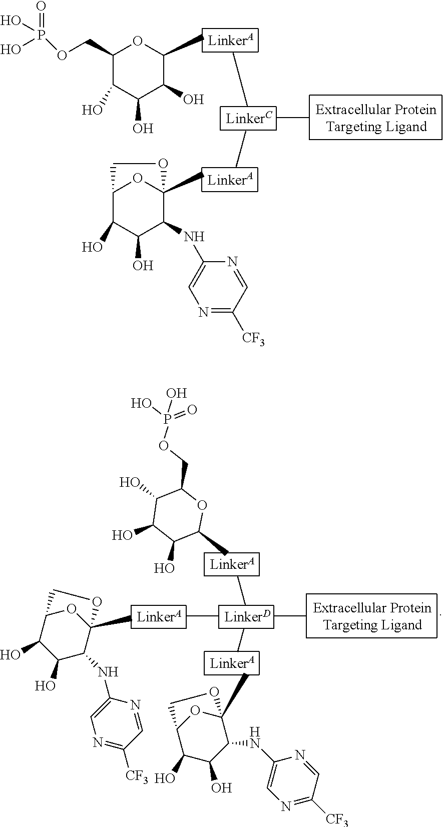

Non-limiting examples of compounds of Formula IV, Formula V, and Formula VI include:

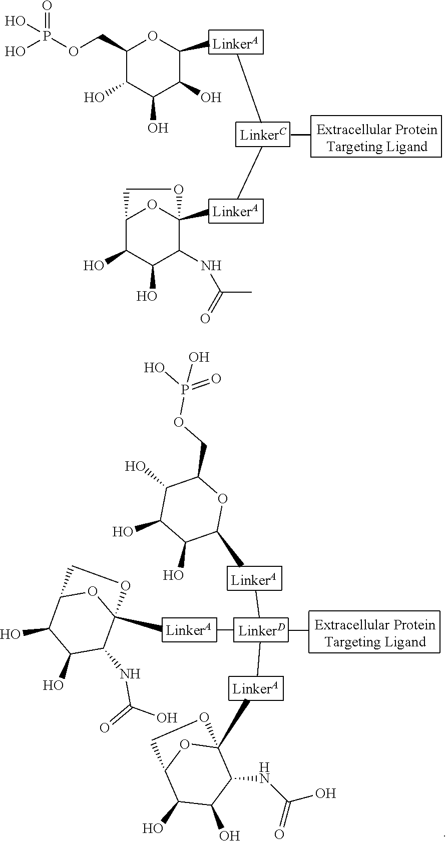

In certain embodiments the ASGPR Ligand is selected from:

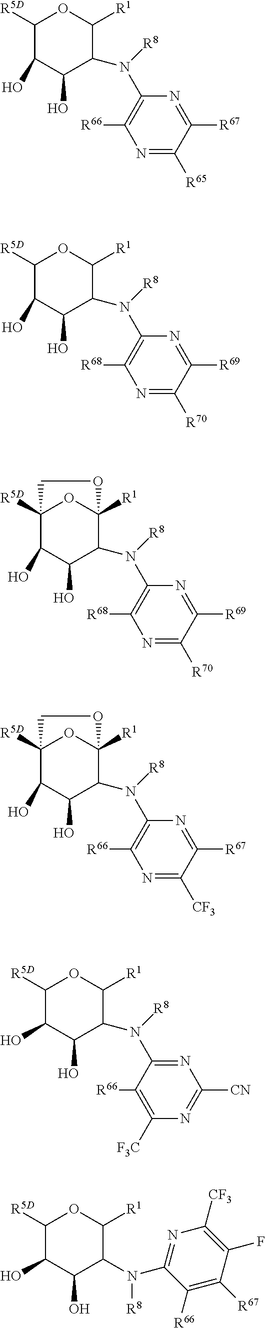

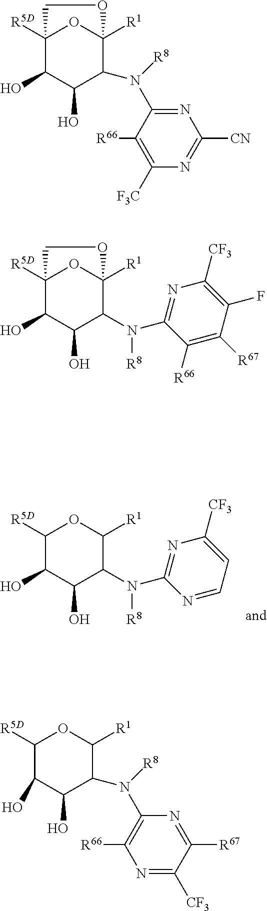

or a pharmaceutically acceptable salt thereof,
wherein:
R¹ and R^{5D} are independently selected from hydrogen, heteroalkyl, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, 3, or 4 substituents;
R⁶⁵, R⁶⁶, and R⁶⁷ are independently selected from hydrogen, heteroalkyl, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, 3, or 4 substituents;
R⁶⁸, R⁶⁹, and R⁷⁰ are independently selected from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)-S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, heteroaryl, aryl, and C₀-C₆alkyl-O—S(O)₂R³ each of which is optionally substituted with 1, 2, 3, or 4 substituents; and
when a compound is “optionally substituted” it may be substituted as allowed by valence with one or more groups selected from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, heteroalkyl, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, —SF₅,

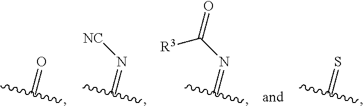

wherein the optional substituent is selected such that a stable compound results;
wherein all other variables are as defined herein.

In certain embodiments the ASGPR Ligand is:

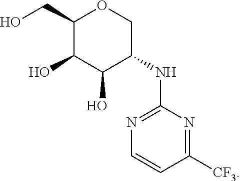 [SMILES: OC[C@H]1OC[C@H](Nc2nccc(C(F)(F)F)n2)[C@@H](O)[C@H]1O]

In certain embodiments the ASGPR Ligand is:

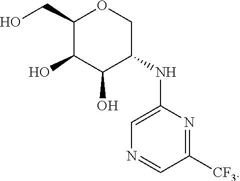 [SMILES: OC[C@H]1OC[C@H](Nc2cncc(C(F)(F)F)n2)[C@@H](O)[C@H]1O]

In other embodiments the ASGPR Ligand is selected from:

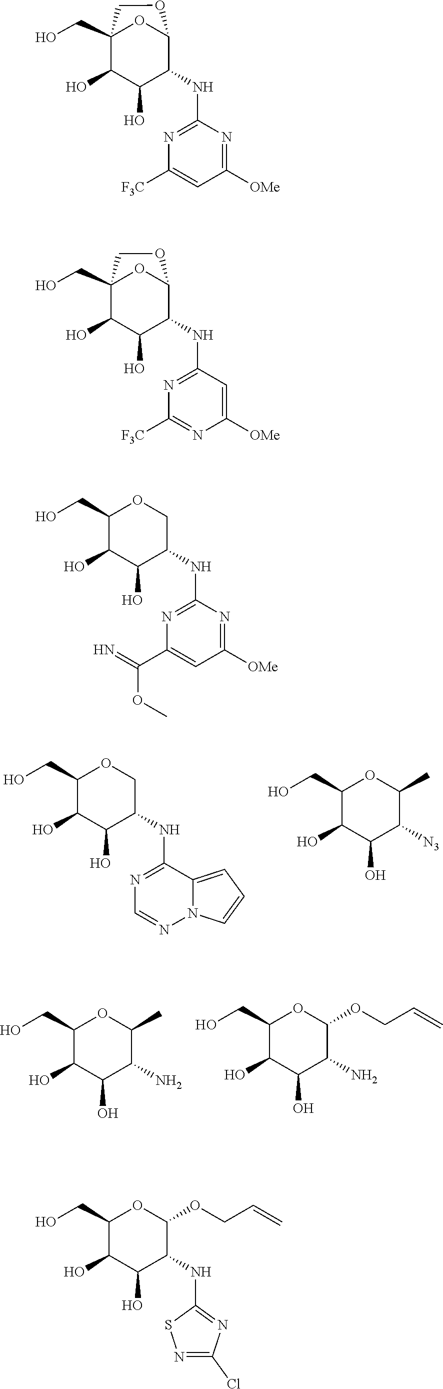

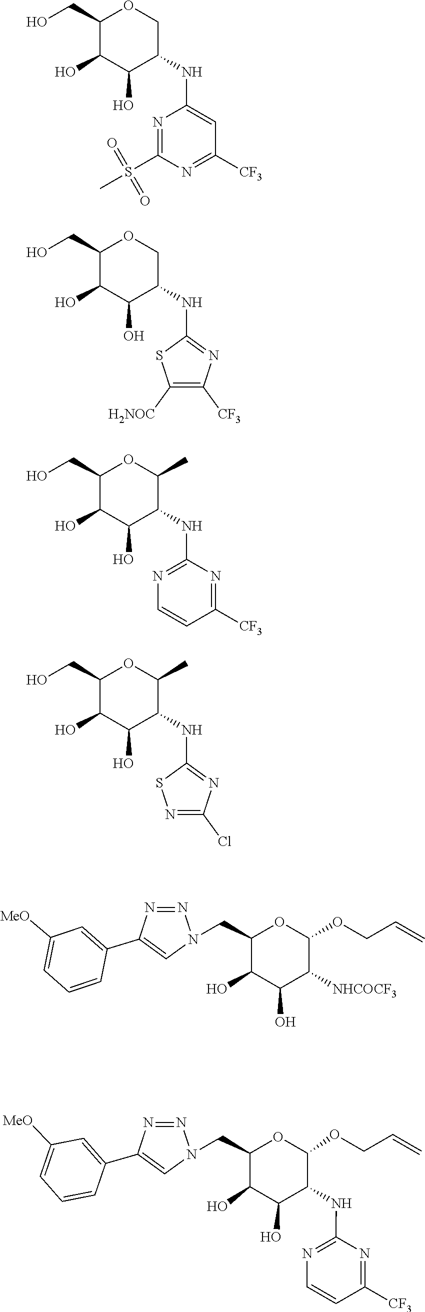

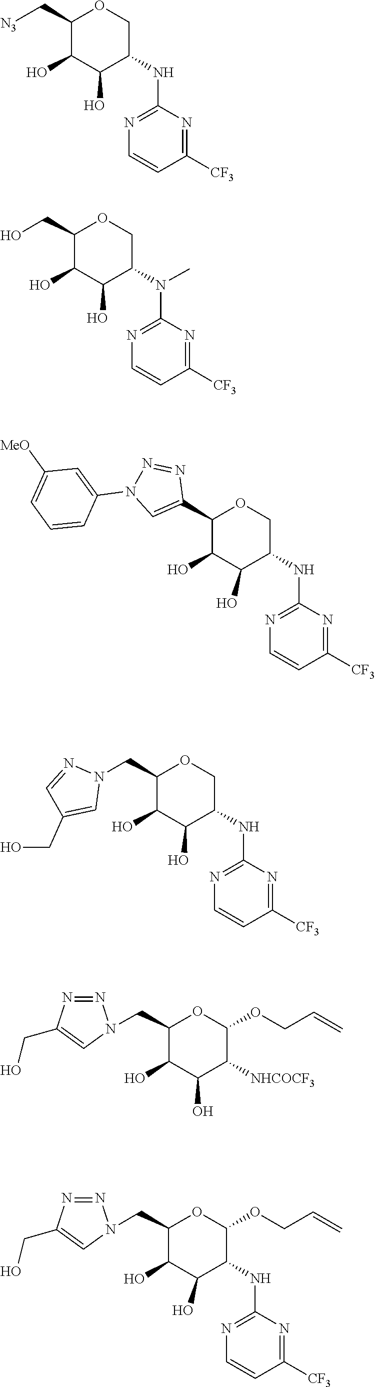

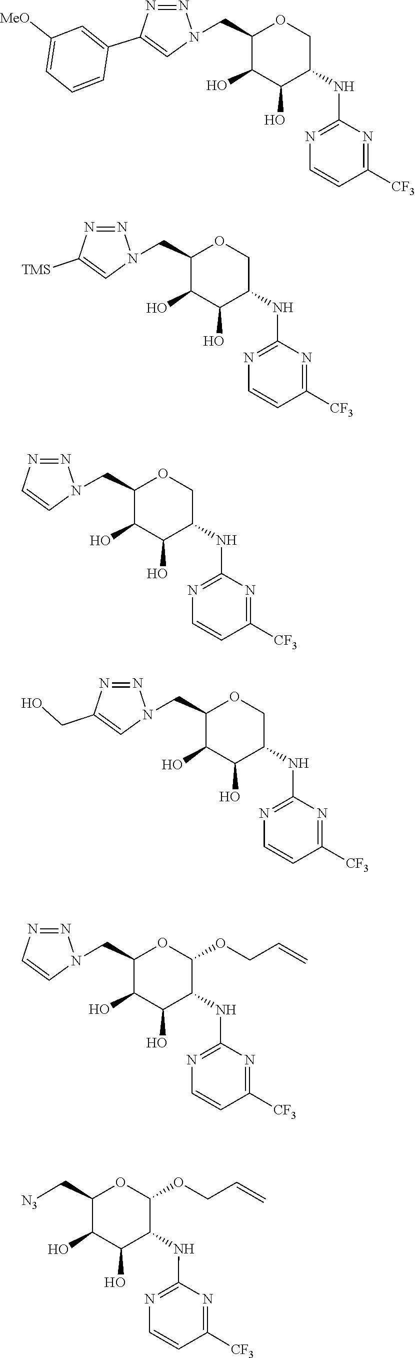 [SMILES: COc1cccc(-c2cn(C[C@H]3OC[C@H](Nc4nccc(C(F)(F)F)n4)[C@@H](O)[C@H]3O)nn2)c1 ; C[Si](C)(C)c1cn(C[C@H]2OC[C@H](Nc3nccc(C(F)(F)F)n3)[C@@H](O)[C@H]2O)nn1 ; O[C@@H]1[C@H](O)[C@@H](Nc2nccc(C(F)(F)F)n2)CO[C@@H]1Cn1ccnn1 ; OCc1cn(C[C@H]2OC[C@H](Nc3nccc(C(F)(F)F)n3)[C@@H](O)[C@H]2O)nn1 ; C=CCO[C@H]1O[C@H](Cn2ccnn2)[C@H](O)[C@H](O)[C@H]1Nc1nccc(C(F)(F)F)n1 ; C=CCO[C@H]1O[C@H](CN=[N+]=[N-])[C@H](O)[C@H](O)[C@H]1Nc1nccc(C(F)(F)F)n1]

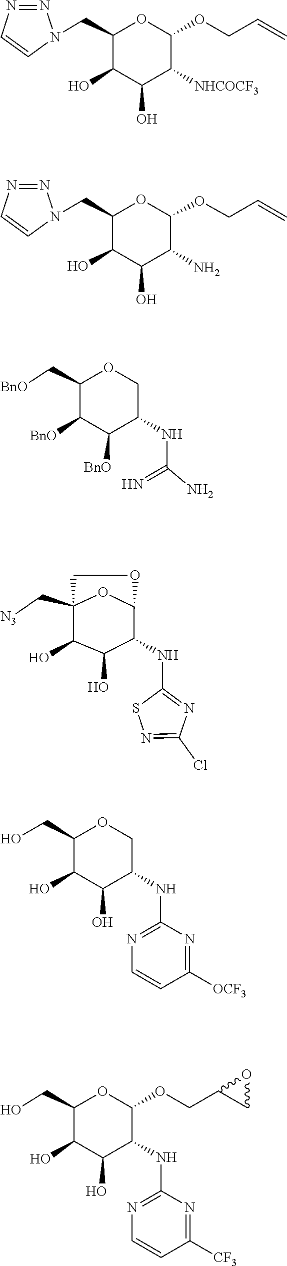

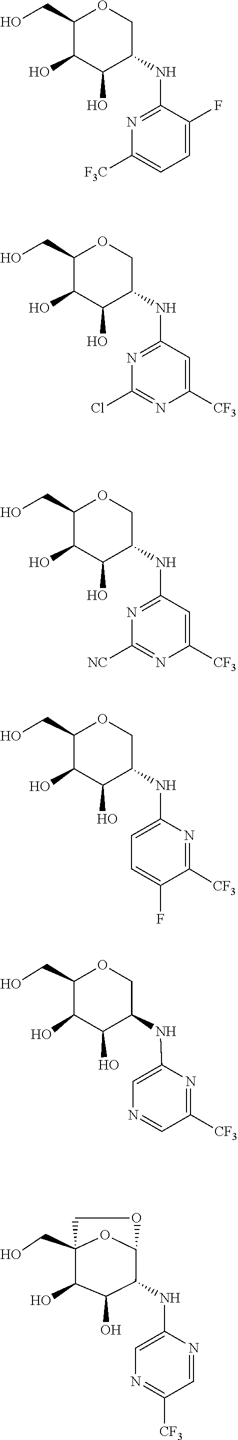 [SMILES: OC[C@H]1OC[C@H](Nc2nc(C(F)(F)F)ccc2F)[C@@H](O)[C@H]1O ; OC[C@H]1OC[C@H](Nc2cc(C(F)(F)F)nc(Cl)n2)[C@@H](O)[C@H]1O ; N#Cc1nc(N[C@H]2CO[C@H](CO)[C@H](O)[C@@H]2O)cc(C(F)(F)F)n1 ; OC[C@H]1OC[C@H](Nc2ccc(F)c(C(F)(F)F)n2)[C@@H](O)[C@H]1O ; OC[C@H]1OC[C@@H](Nc2cncc(C(F)(F)F)n2)[C@@H](O)[C@H]1O ; OC[C@@]12CO[C@@H](O1)[C@H](Nc1cnc(C(F)(F)F)cn1)[C@@H](O)[C@H]2O]

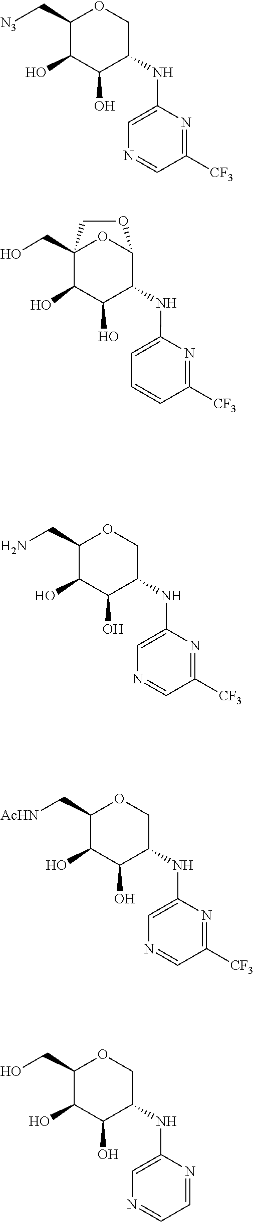 [SMILES: [N-]=[N+]=NC[C@H]1OC[C@H](Nc2cncc(C(F)(F)F)n2)[C@@H](O)[C@H]1O ; OC[C@@]12CO[C@@H](O1)[C@H](Nc1cccc(C(F)(F)F)n1)[C@@H](O)[C@H]2O ; NC[C@H]1OC[C@H](Nc2cncc(C(F)(F)F)n2)[C@@H](O)[C@H]1O ; CC(=O)NC[C@H]1OC[C@H](Nc2cncc(C(F)(F)F)n2)[C@@H](O)[C@H]1O ; OC[C@H]1OC[C@H](Nc2cnccn2)[C@@H](O)[C@H]1O]

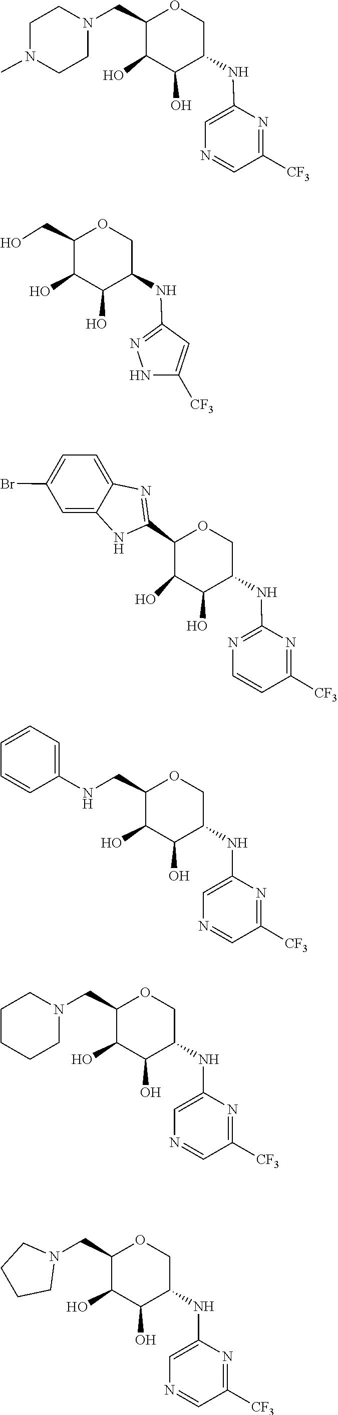 [SMILES: CN1CCN(C[C@H]2OC[C@H](Nc3cncc(C(F)(F)F)n3)[C@@H](O)[C@H]2O)CC1 ; OC[C@H]1OC[C@@H](Nc2cc(C(F)(F)F)[nH]n2)[C@@H](O)[C@H]1O ; O[C@@H]1[C@H](O)[C@@H](Nc2nccc(C(F)(F)F)n2)CO[C@@H]1c1nc2ccc(Br)cc2[nH]1 ; O[C@@H]1[C@H](O)[C@@H](Nc2cncc(C(F)(F)F)n2)CO[C@@H]1CNc1ccccc1 ; O[C@@H]1[C@H](O)[C@@H](Nc2cncc(C(F)(F)F)n2)CO[C@@H]1CN1CCCCC1 ; O[C@@H]1[C@H](O)[C@@H](Nc2cncc(C(F)(F)F)n2)CO[C@@H]1CN1CCCC1]

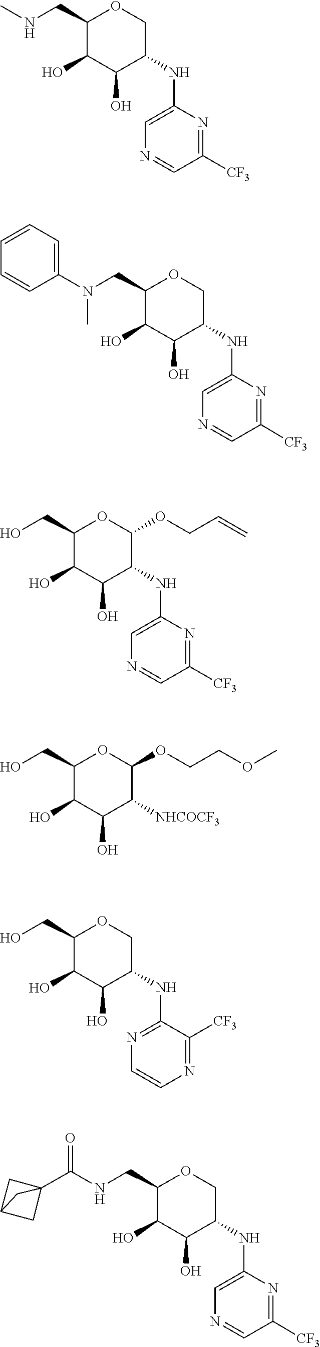

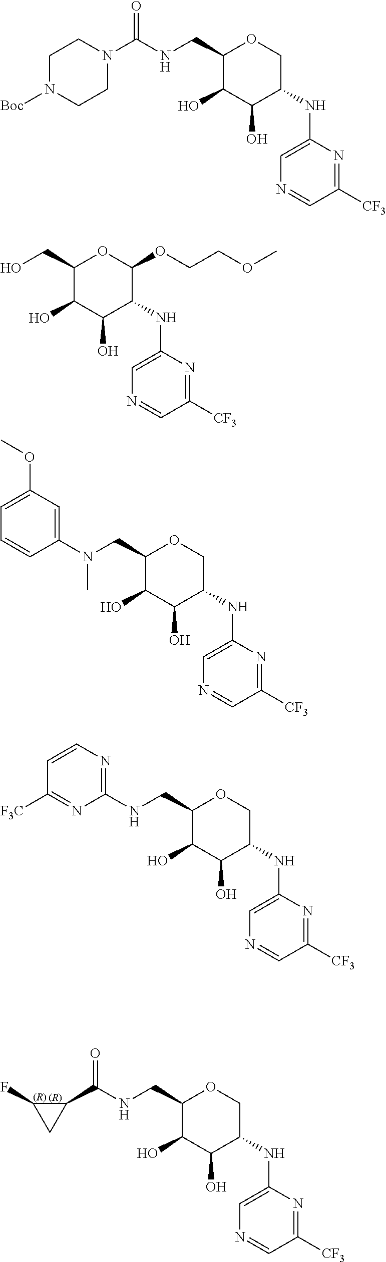

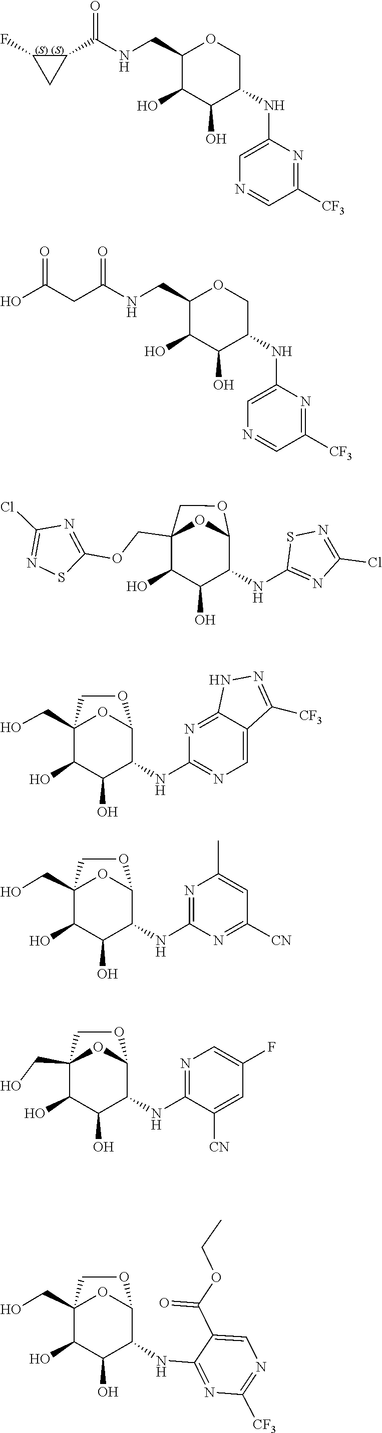

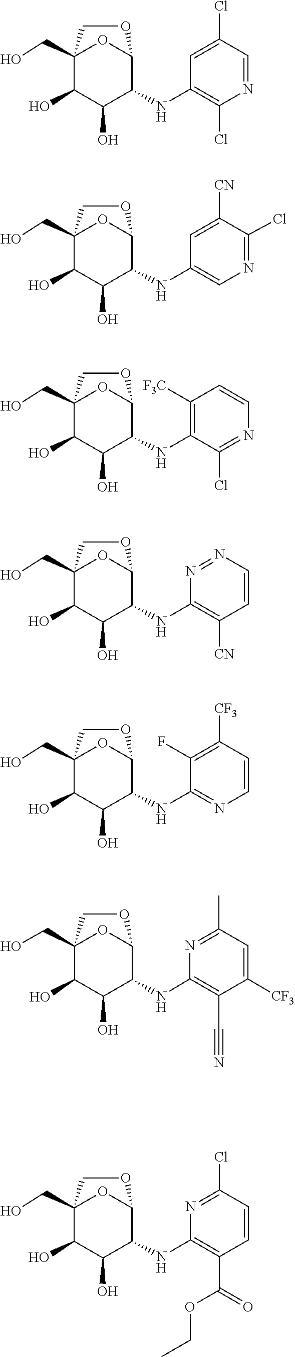

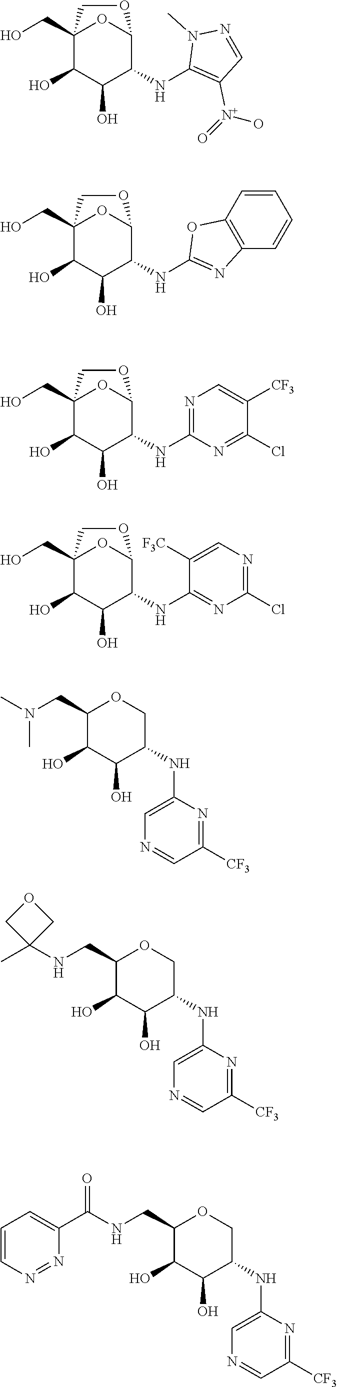

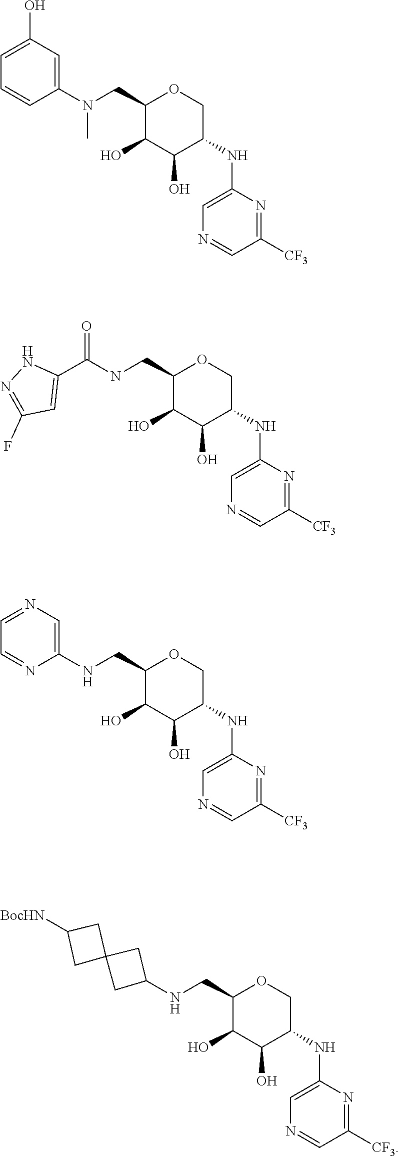

### VII. Embodiments of the Linker

In non-limiting embodiments, Linker^{A} and Linker^{B} are independently selected from:

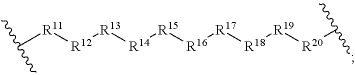

wherein:
R¹¹, R¹², R¹³, R¹⁴, R¹⁵, R¹⁶, R¹⁷, R⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —C(O)N(OH)—, —N(OH)C(O)—, —O—, —S—, —NR⁶—, —C(R²¹R²¹)—, —S(O)(═N—R⁴⁴)—, —S(O)(═NR), —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, aryl, heteroaryl, and heterocycle;
and the remaining variables are as defined herein.

In one embodiment Linker^{A} is bond and Linker^{B} is

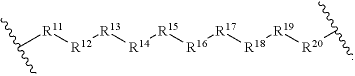

In one embodiment Linker^{B} is bond and Linker^{A} is

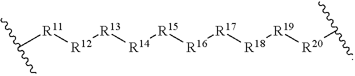

In one embodiment, a divalent residue of an amino acid is selected from

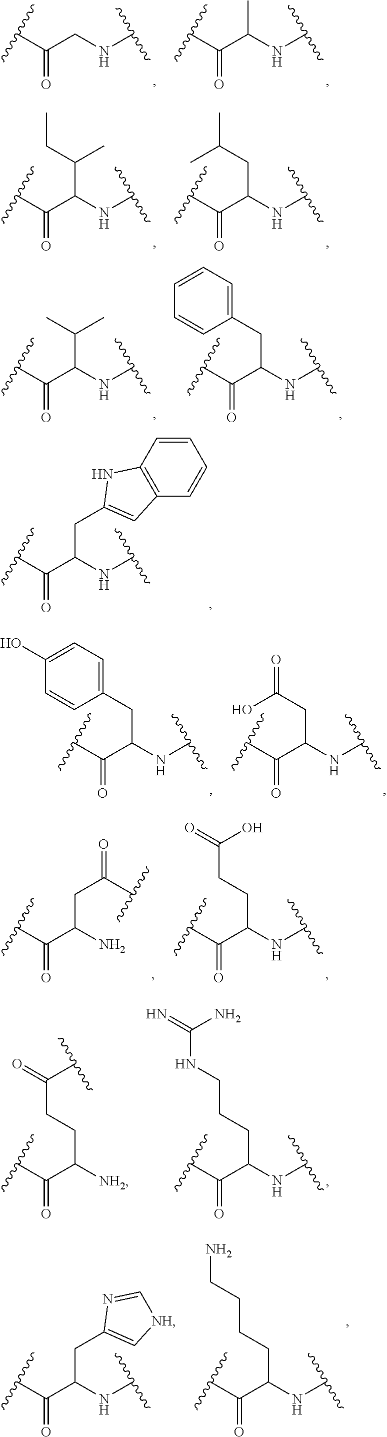

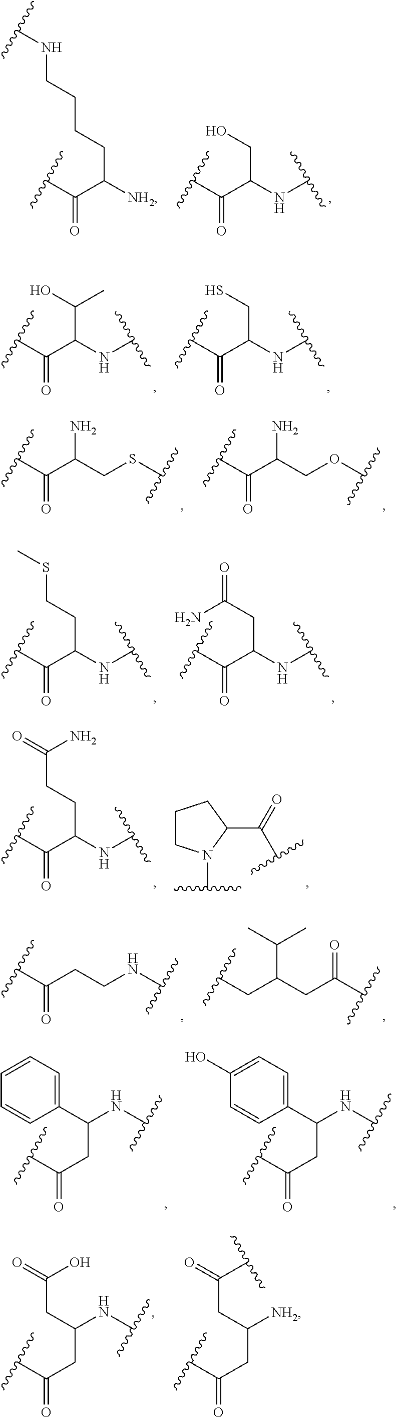

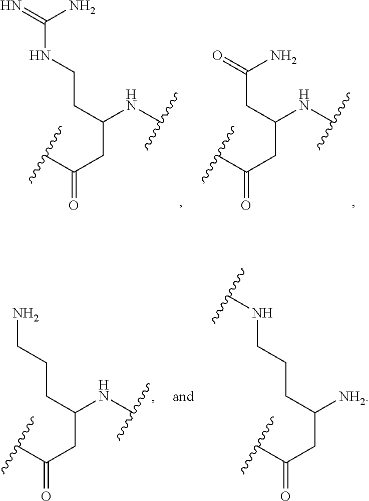 [SMILES: *NC(CCNC(=N)N)CC(*)=O ; *NC(CC(*)=O)CC(N)=O ; *NC(CCCN)CC(*)=O ; *NCCCC(N)CC(*)=O]

wherein the amino acid can be oriented in either direction and wherein the amino acid can be in the L- or D-form or a mixture thereof.

In one embodiment, a divalent residue of a dicarboxylic acid is generated from a nucleophilic addition reaction:

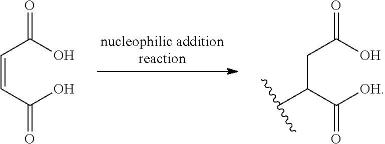 [SMILES: O=C(O)/C=C\C(=O)O ; *C(CC(=O)O)C(=O)O]

Non-limiting embodiments of a divalent residue of a dicarboxylic acid generated from a nucleophilic addition reaction include:

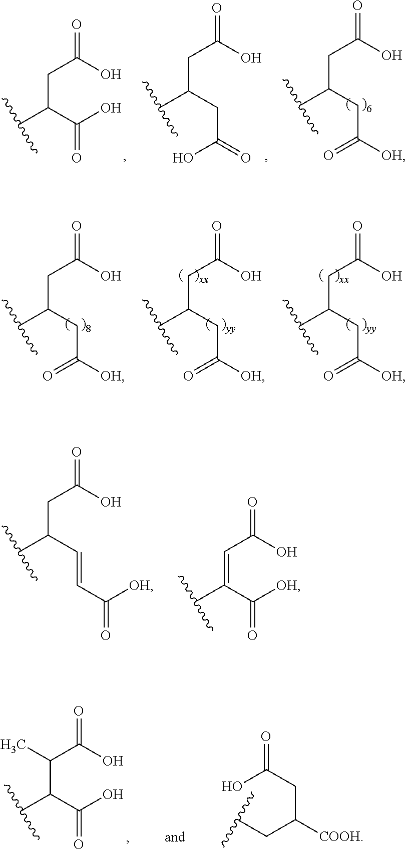

In one embodiment, a divalent residue of a dicarboxylic acid is generated from a condensation reaction:

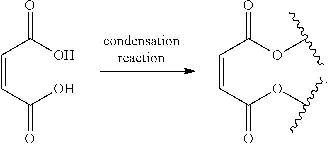 [SMILES: O=C(O)/C=C\C(=O)O ; *OC(=O)/C=C\C(=O)O*]

Non-limiting embodiments of a divalent residue of a dicarboxylic acid generated from a condensation include:

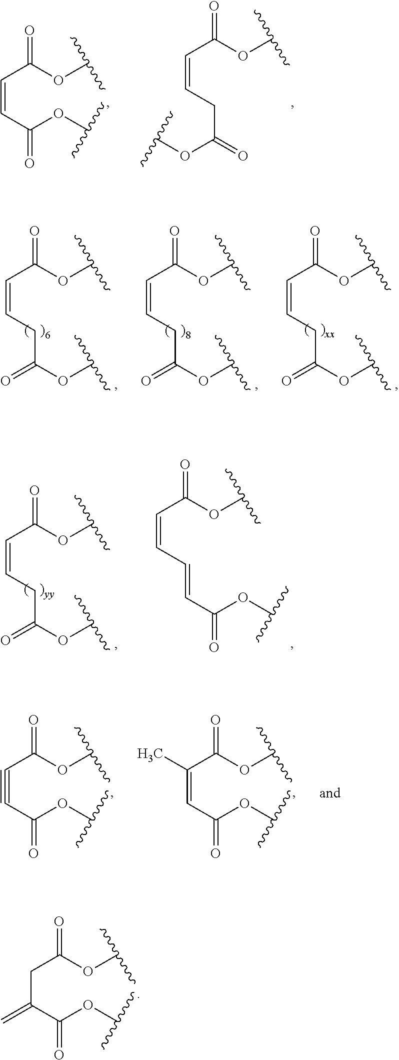

Non-limiting embodiments of a divalent residue of a saturated dicarboxylic acid include:

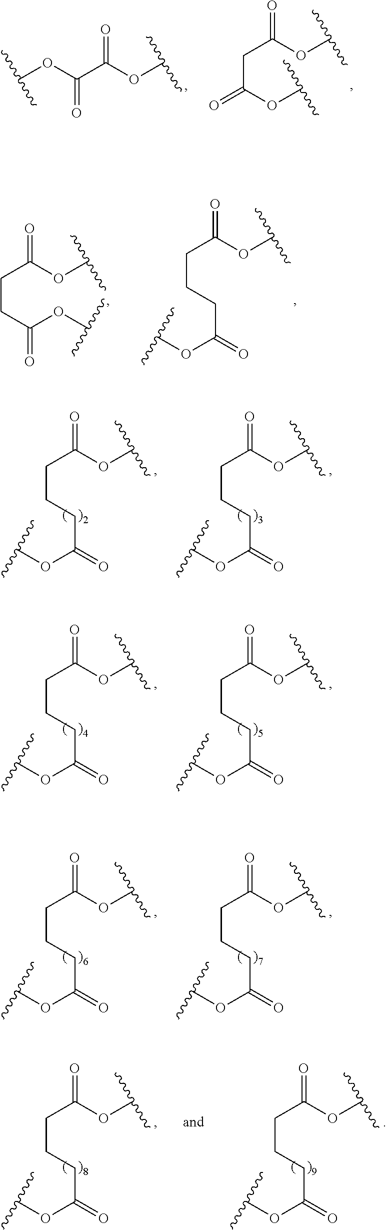

Non-limiting embodiments of a divalent residue of a saturated dicarboxylic acid include:

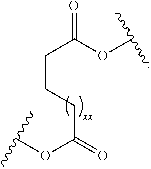

Non-limiting embodiments of a divalent residue of a saturated monocarboxylic acid is selected from butyric acid (—OC(O)(CH₂)₂CH₂—), caproic acid (—OC(O)(CH₂)₄CH₂—), caprylic acid (—OC(O)(CH₂)₅CH₂—), capric acid (—OC(O)(CH₂)₈CH₂—), lauric acid (—OC(O)(CH₂)₁₀CH₂—), myristic acid (—OC(O)(CH₂)₁₂CH₂—), pentadecanoic acid (—OC(O)(CH₂)₁₃CH₂—), palmitic acid (—OC(O)(CH₂)₁₄CH₂—), stearic acid (—OC(O)(CH₂)₁₆CH₂—), behenic acid (—OC(O)(CH₂)₂₀CH₂—), and lignoceric acid (—OC(O)(CH₂)₂₂CH₂—);

Non-limiting embodiments of a divalent residue of a fatty acid include residues selected from linoleic acid, palmitoleic acid, vaccenic acid, paullinic acid, oleic acid, elaidic acid, gondoic acid, gadoleic acid, nervonic acid, myristoleic acid, and erucic acid:

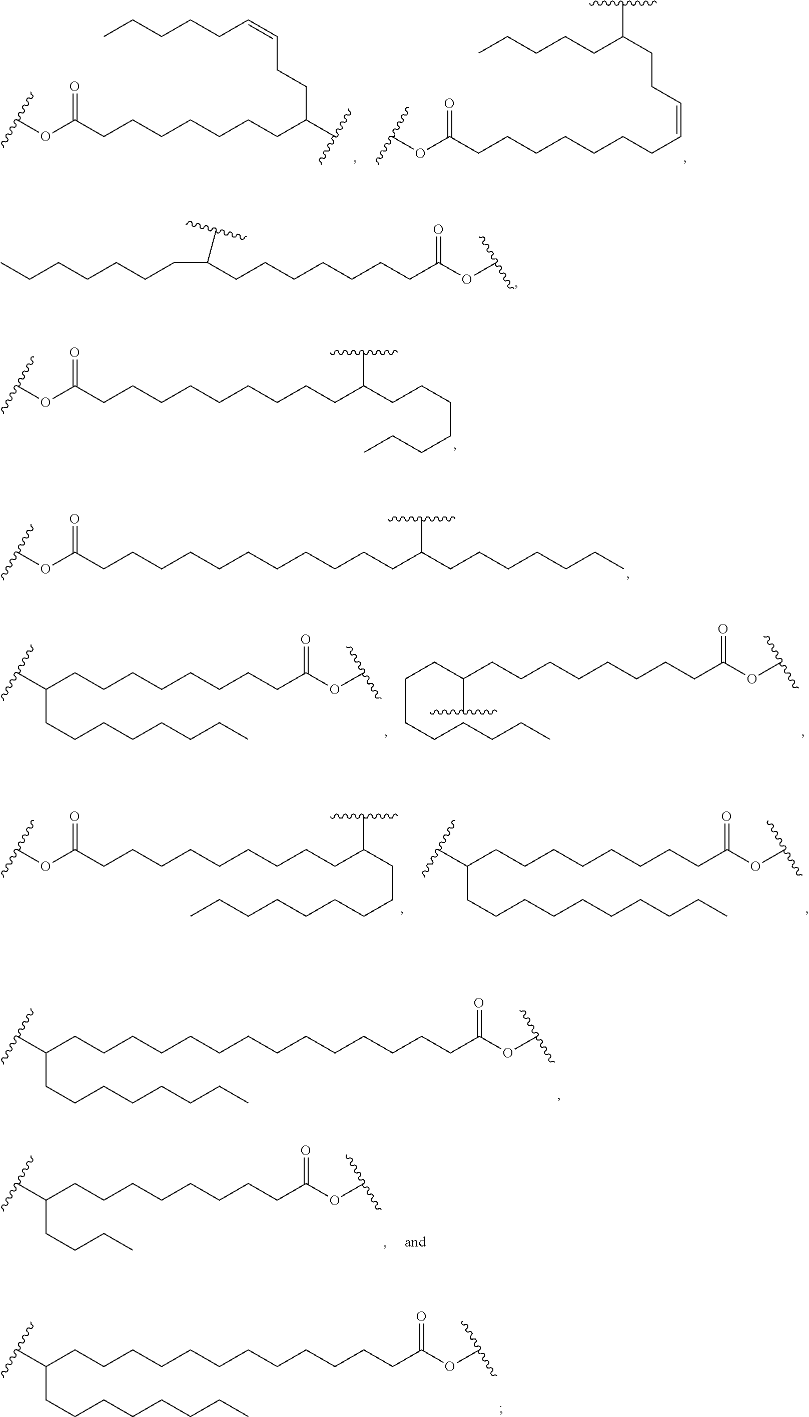

Non-limiting embodiments of a divalent residue of a fatty acid is selected from linoleic acid (—C(O)(CH₂)₇(CH)₂CH₂(CH)₂(CH₂)₄CH₂—), docosahexaenoic acid (—C(O)(CH₂)₂(CHCHCH₂)₆CH₂—), eicosapentaenoic acid (—C(O)(CH₂)₃(CHCHCH₂)₅CH₂—), alpha-linolenic acid (—C(O)(CH₂)₇(CHCHCH₂)₃CH₂—) stearidonic acid (—C(O)(CH₂)₄(CHCHCH₂)₄CH₂—), γ-linolenic acid (—C(O)(CH₂)₄(CHCHCH₂)₃(CH₂)₃CH₂—), arachidonic acid (—C(O)(CH₂)₃, (CHCHCH₂)₄(CH₂)₄CH₂—), docosatetraenoic acid (—C(O)(CH₂)₅(CHCHCH₂)₄(CH₂)₄CH₂—), palmitoleic acid (—C(O)(CH₂)₇CHCH(CH₂)₅CH₂—), vaccenic acid (—C(O)(CH₂)₉CHCH(CH₂)₅CH₂—), paullinic acid (—C(O)(CH₂)₁₁CHCH(CH₂)₅CH₂—), oleic acid (—C(O)(CH₂)₇CHCH(CH₂)₇CH₂—), elaidic acid (—C(O)(CH₂)₇CHCH(CH₂)₇CH₂—), gondoic acid (—C(O)(CH₂)₉CHCH(CH₂)₇CH₂—), gadoleic acid (—C(O)(CH₂)₇CHCH(CH₂)₉CH₂—), nervonic acid (—C(O)(CH₂)₁₃CHCH(CH₂)₇CH₂—), mead acid (—C(O)(CH₂)₃(CHCHCH₂)₃(CH₂)₆CH₂—), myristoleic acid (—C(O)(CH₂)₇CHCH(CH₂)₃CH₂—), and erucic acid (—C(O)(CH₂)₁₁CHCH(CH₂)₇CH₂—).

In certain embodiments Linker^{C} is selected from:

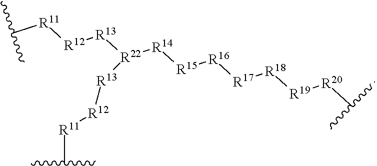

wherein:
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
and the remaining variables are as defined herein.

In certain embodiments Linker^{D} is selected from:

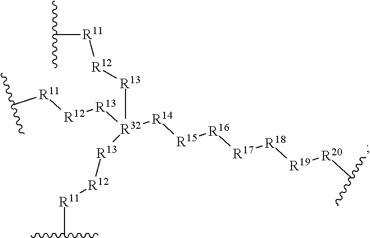

wherein:
R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹;
X⁻ is an anionic group, for example Br⁻ or Cl⁻; and
all other variables are as defined herein.

In certain embodiments the Linker^{A} is selected from

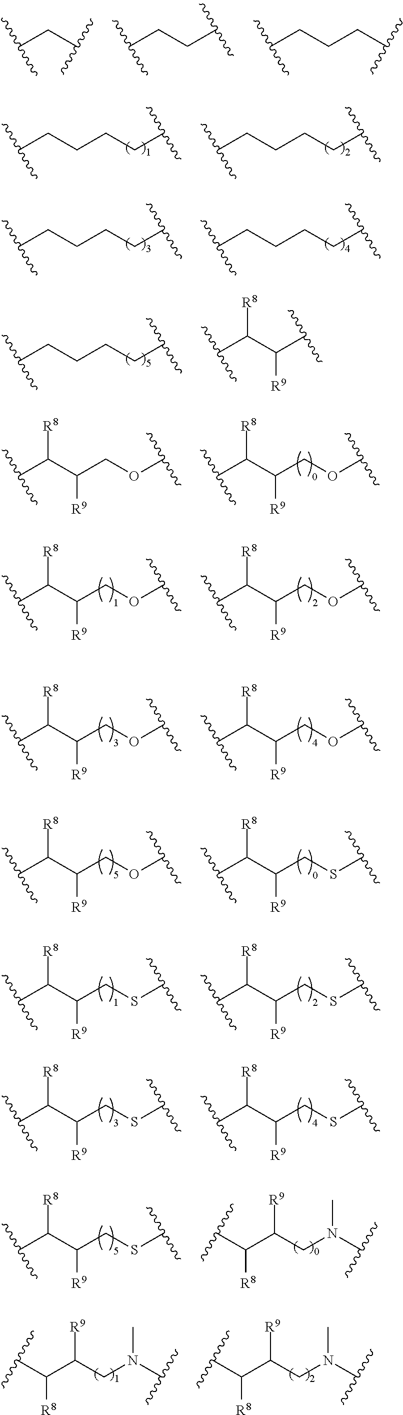

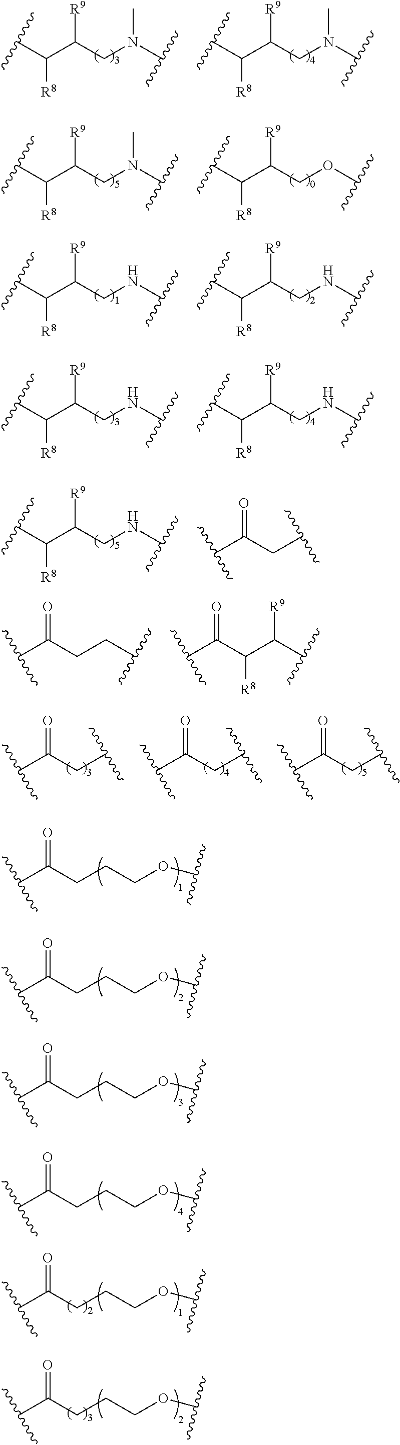

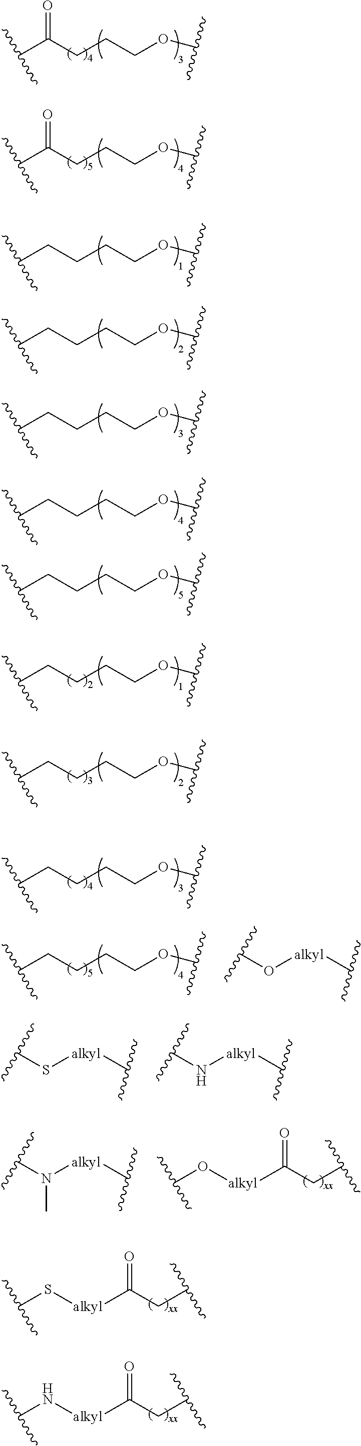

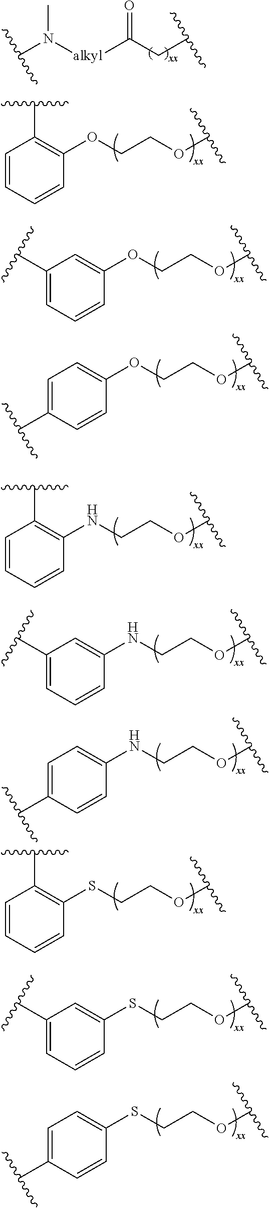

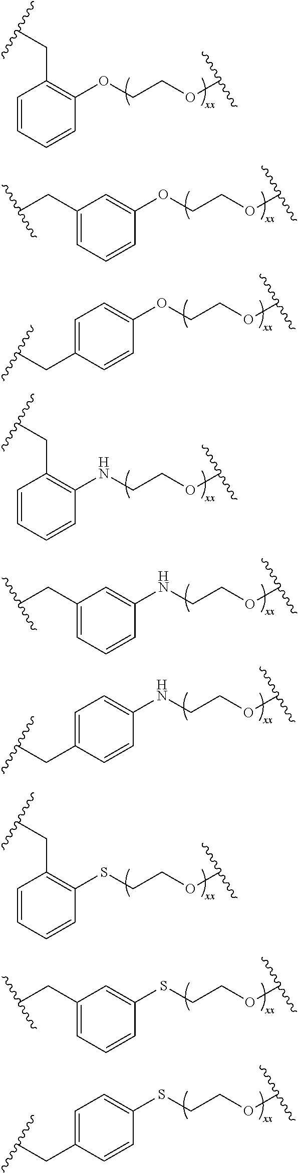

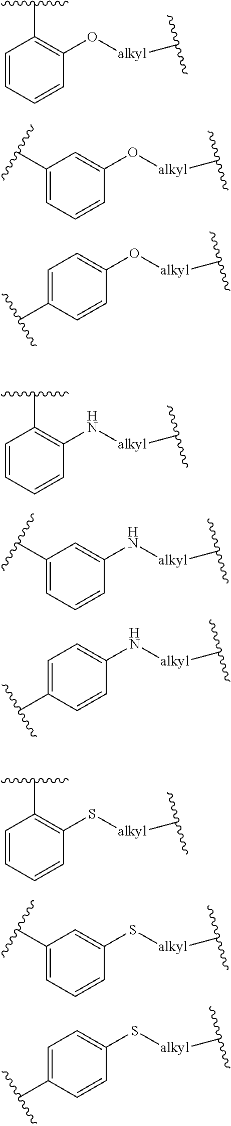

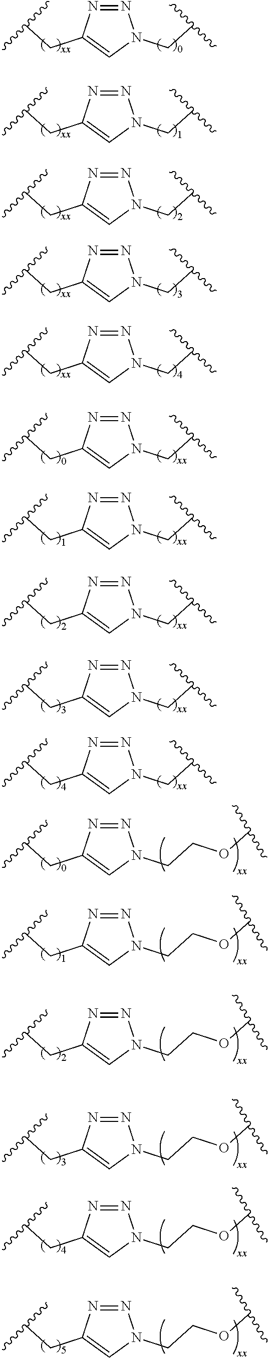

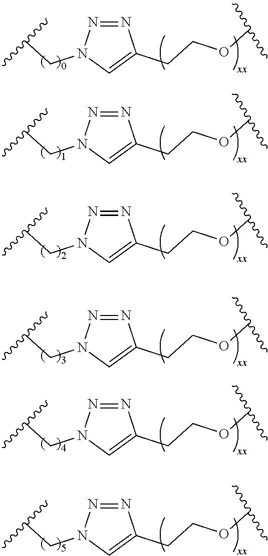

In certain embodiments Linker^{A} is selected from:

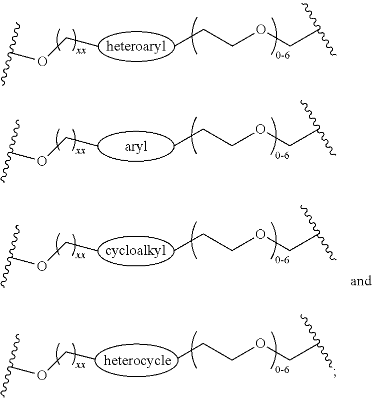

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

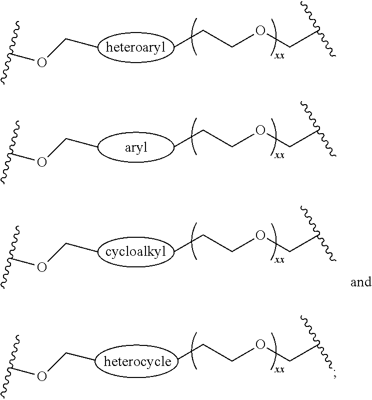

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

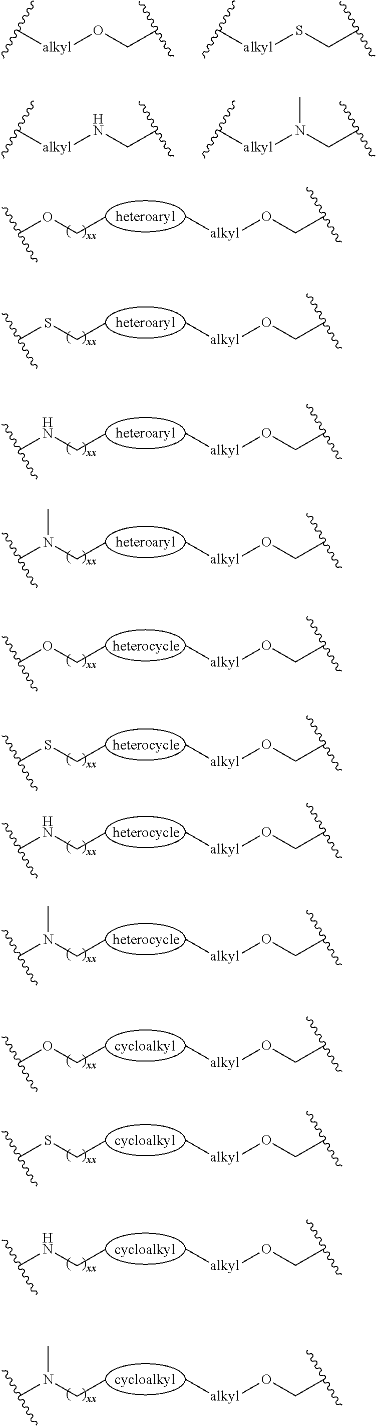

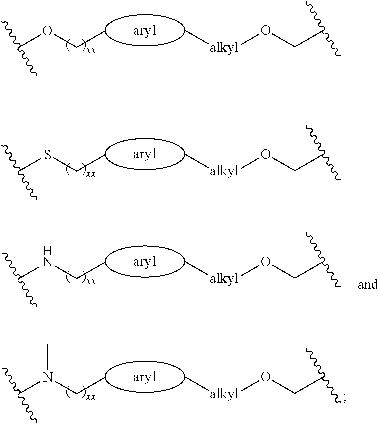

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B} is selected from:

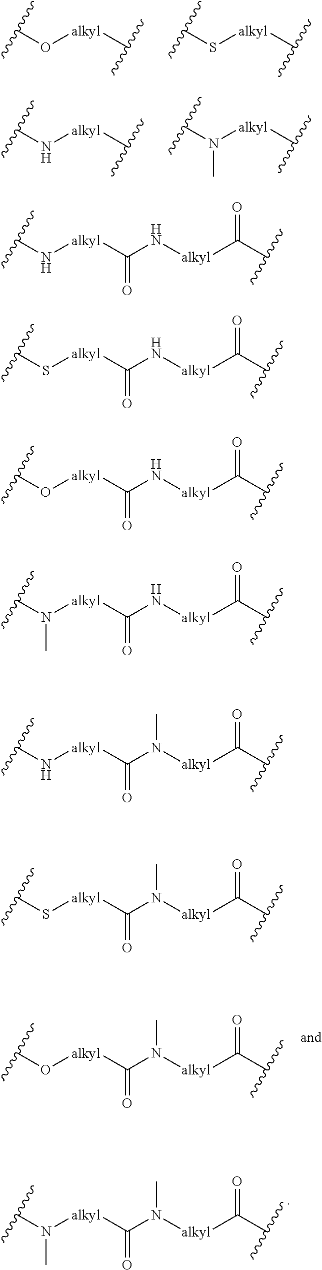

In certain embodiments Linker^{B} is selected from:

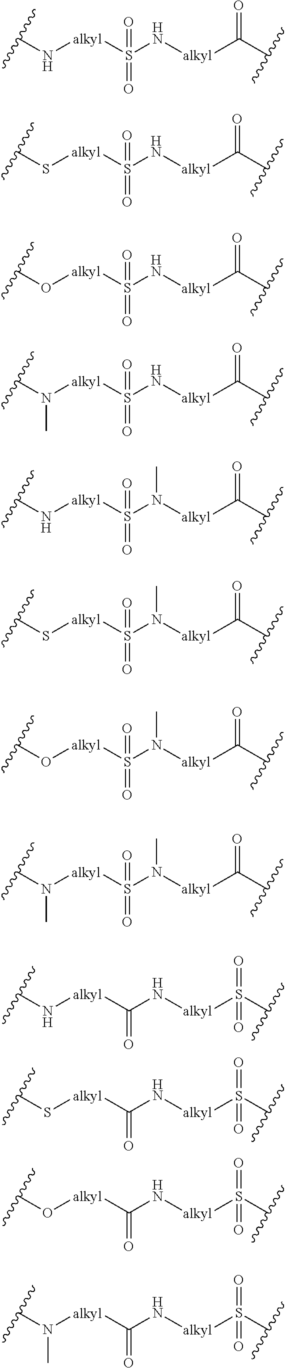

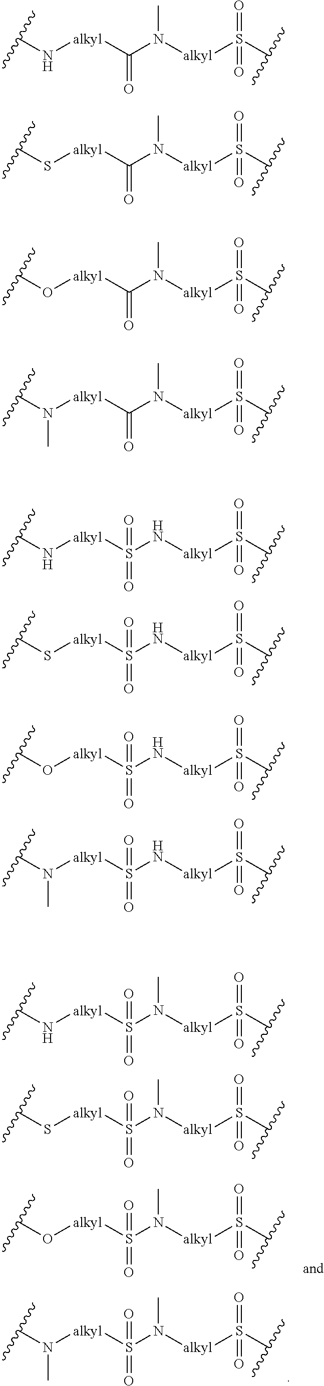

In certain embodiments Linker^{B}, Linker^{C}, or Linker^{D} is selected from:

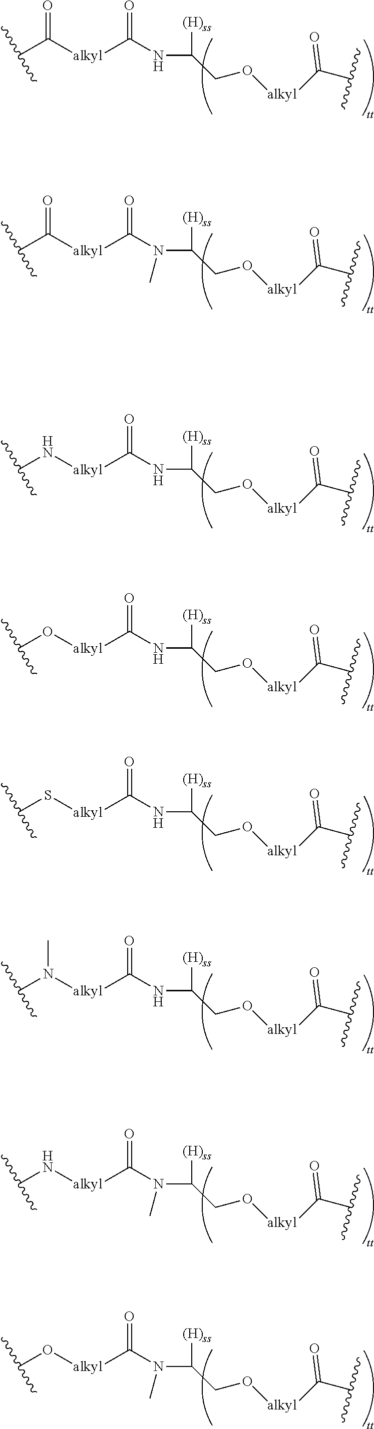

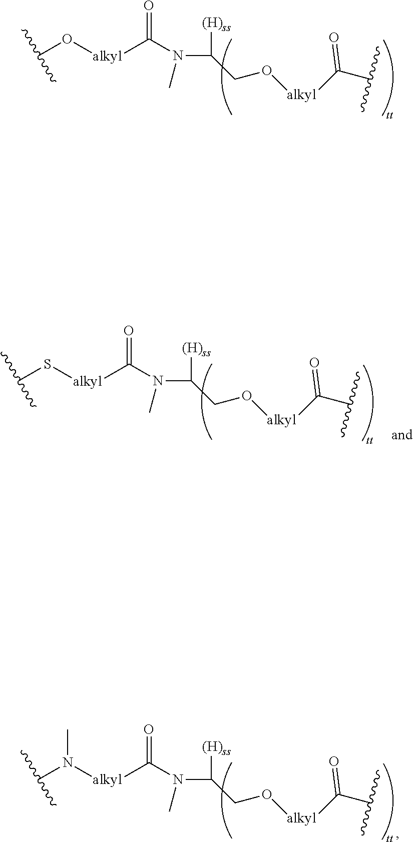

wherein tt is independently selected from 1, 2, or 3 and ss is 3 minus tt.

In certain embodiments Linker^{B}, Linker^{C}, or Linker^{D} is selected from:

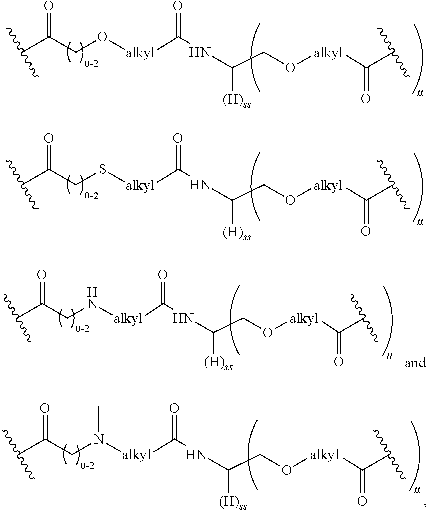

wherein tt and ss are as defined herein.

In certain embodiments Linker^{B} Linker^{C} or Linker^{D} is selected from:

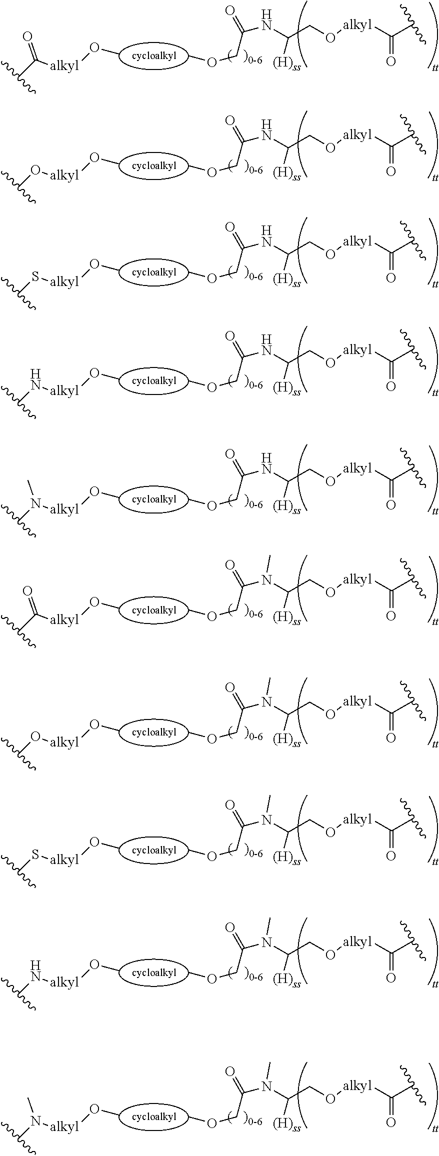

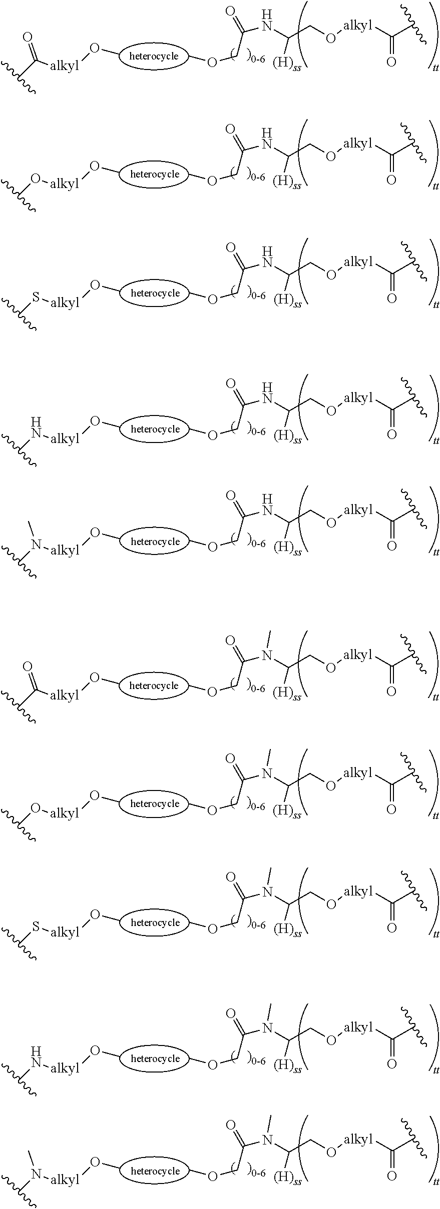

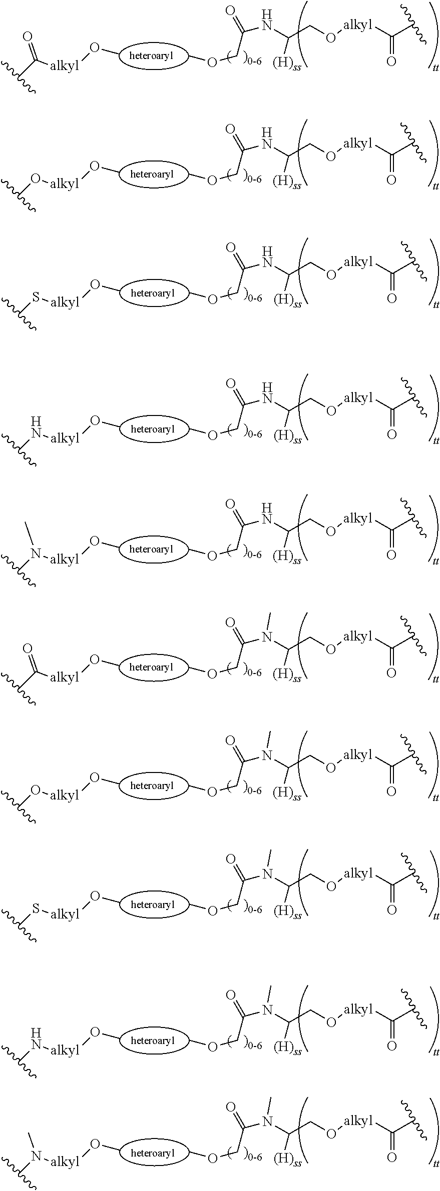

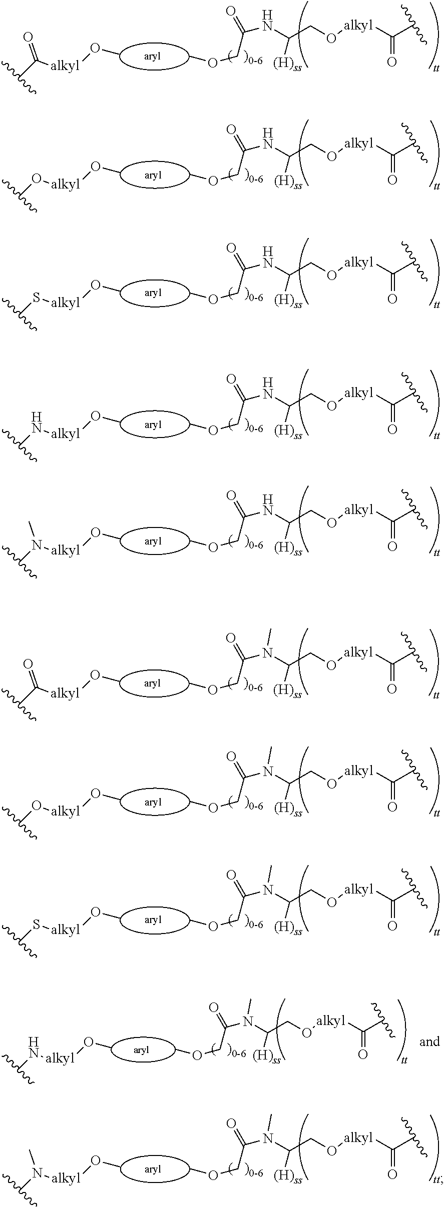

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{B}, Linker^{C}, or Linker^{D} is selected from:

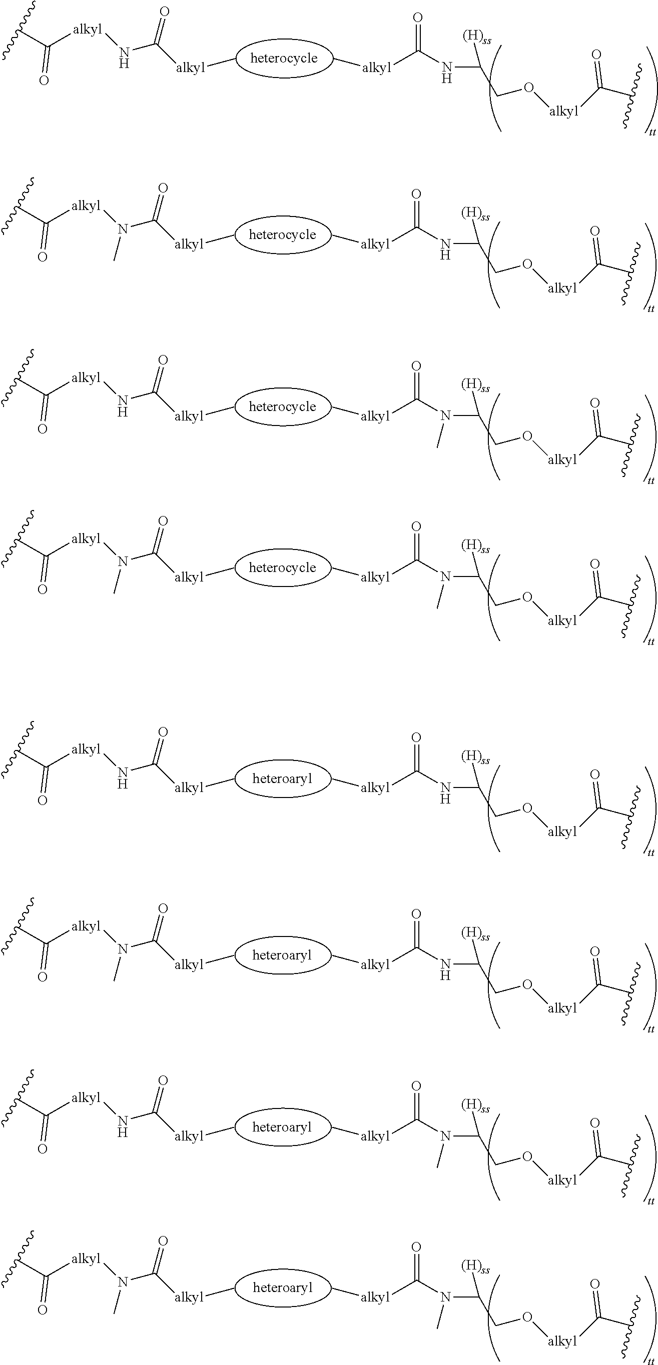

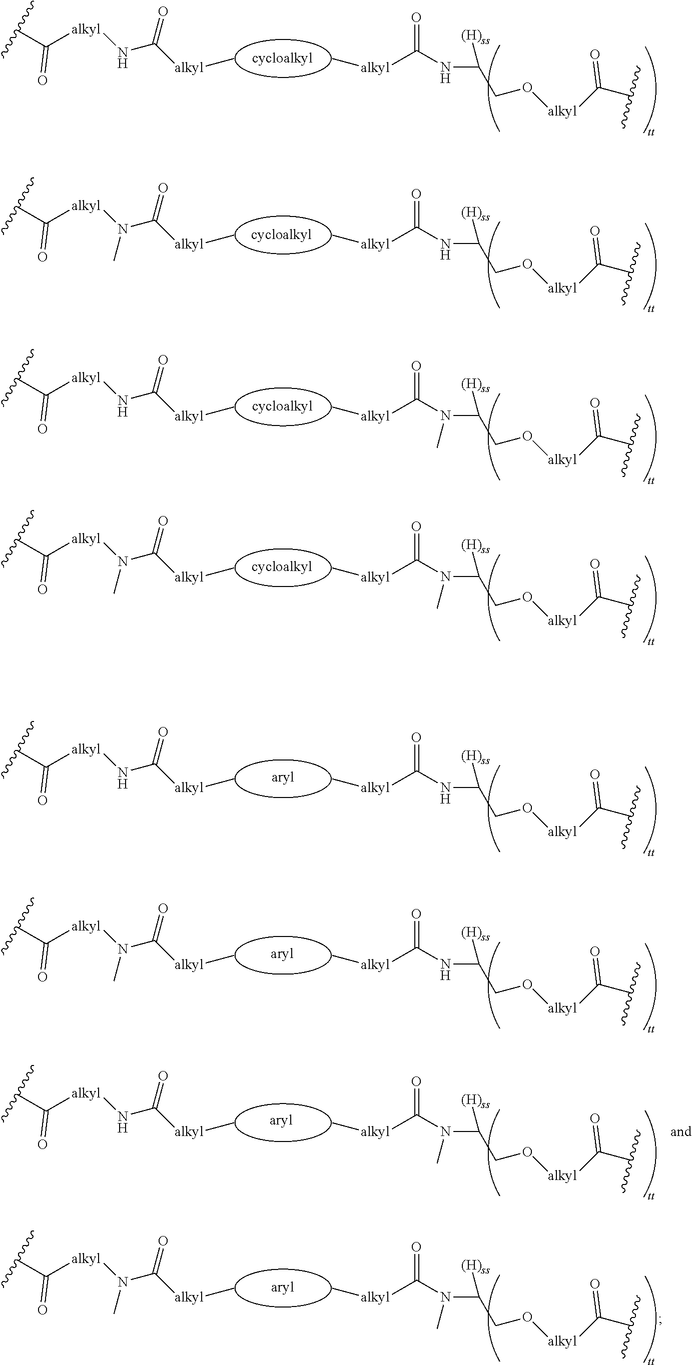

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{B}, Linker^{C}, or Linker^{D} is selected from:

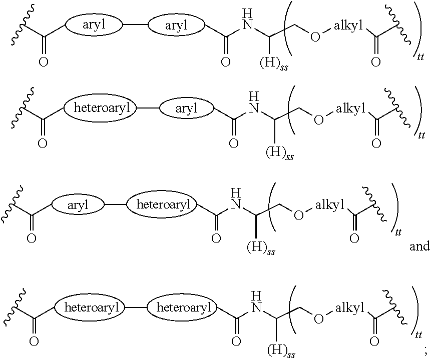

wherein each heteroaryl and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{A} is selected from:

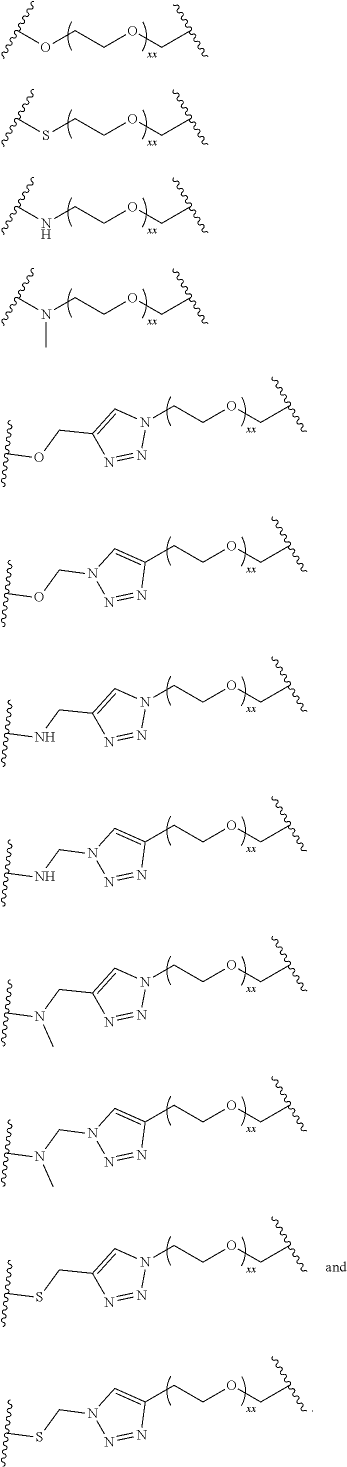

In certain embodiments Linker^{A} is selected from:

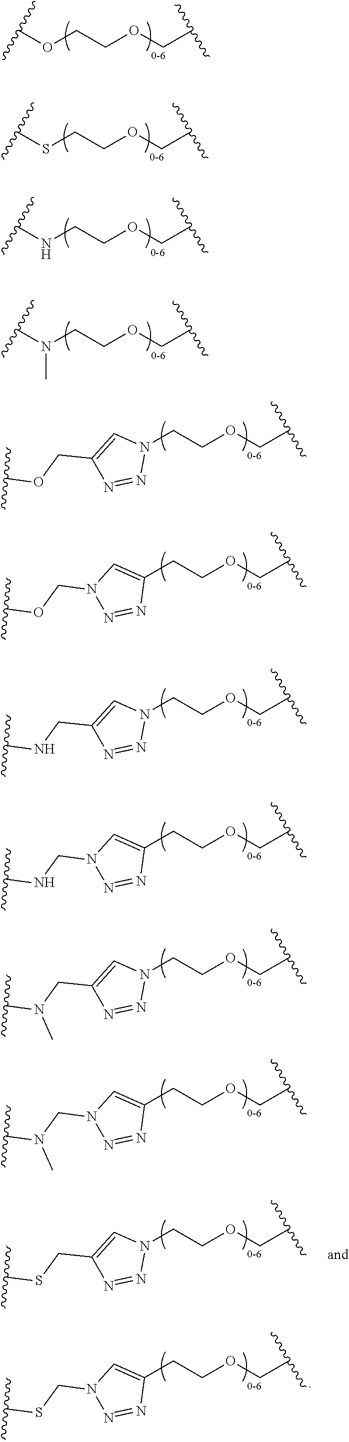

In certain embodiments Linker^{A} is selected from:

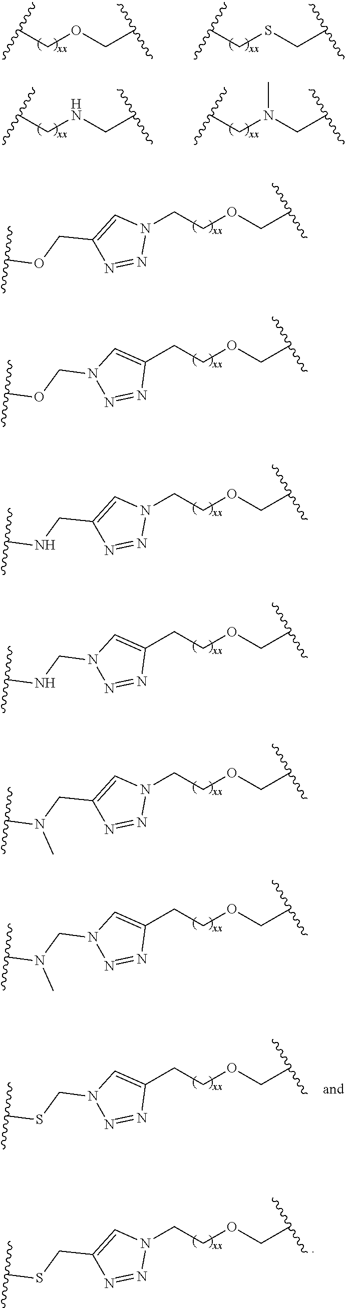

In certain embodiments Linker^{A} is selected from:

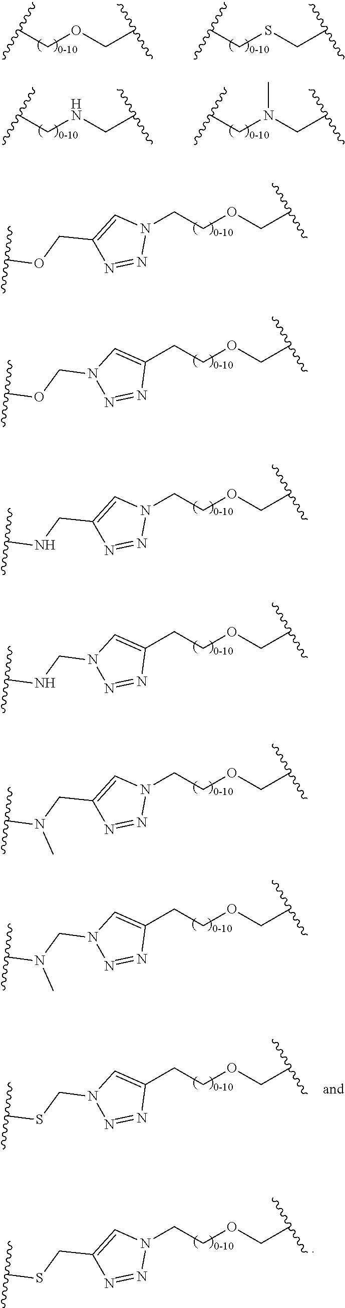

In certain embodiments Linker^{B} is selected from:

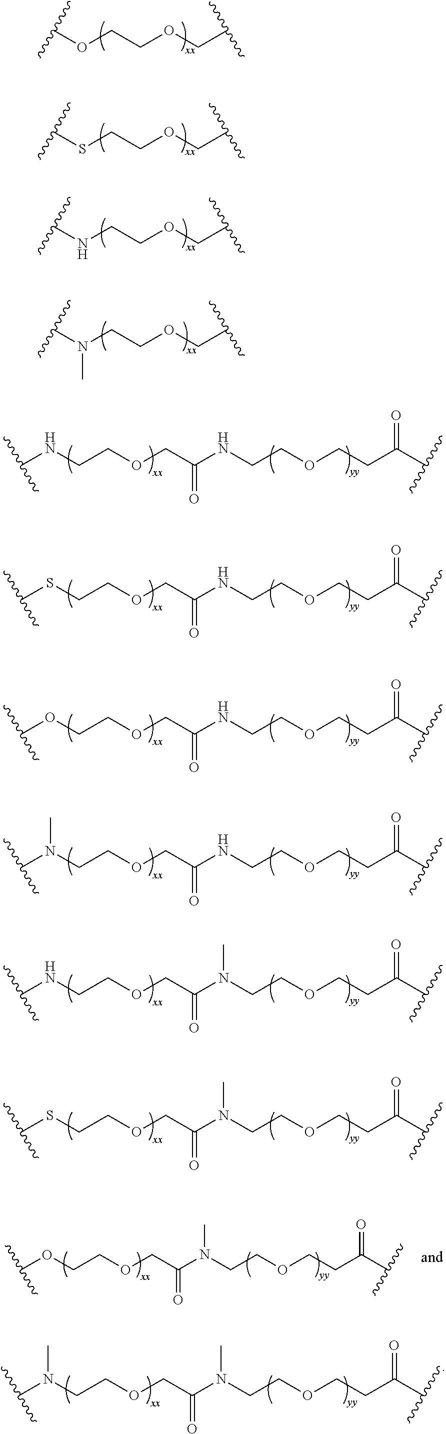

In certain embodiments Linker^{B} is selected from:

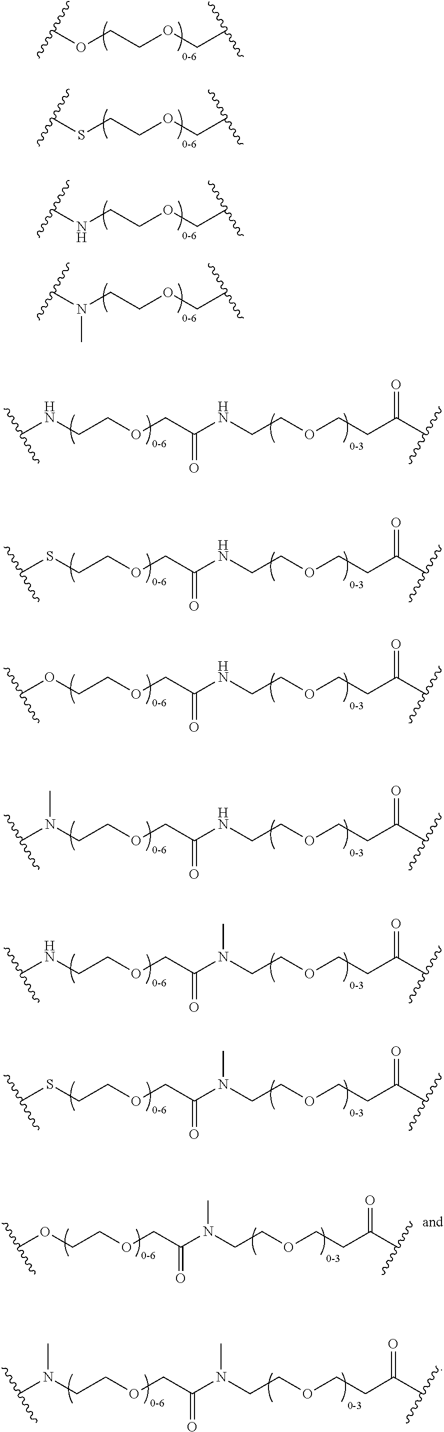

In certain embodiments Linker^{B} is selected from:

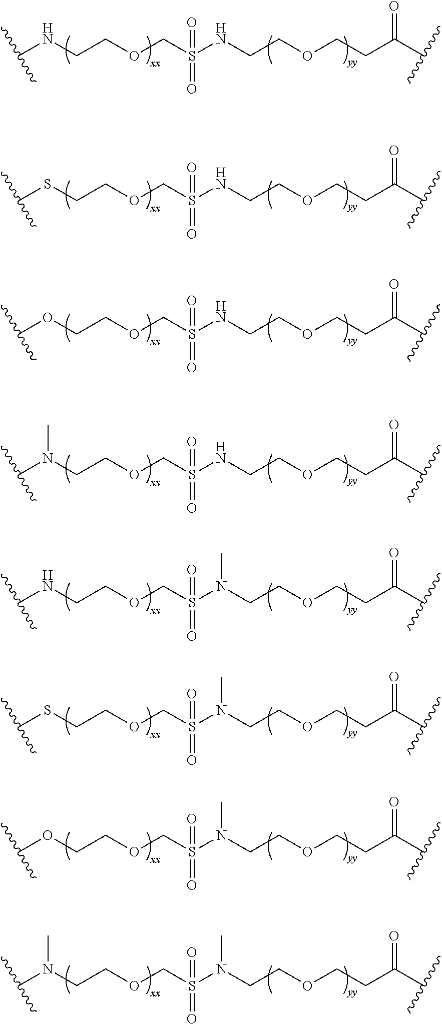

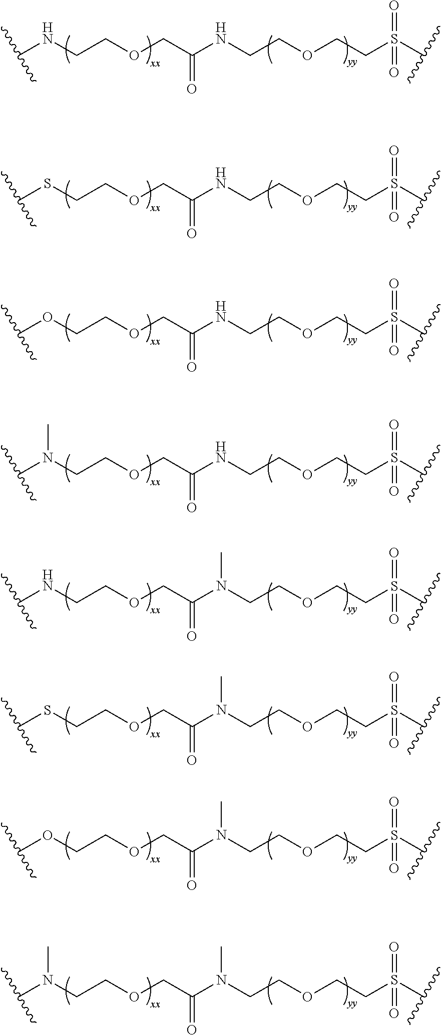

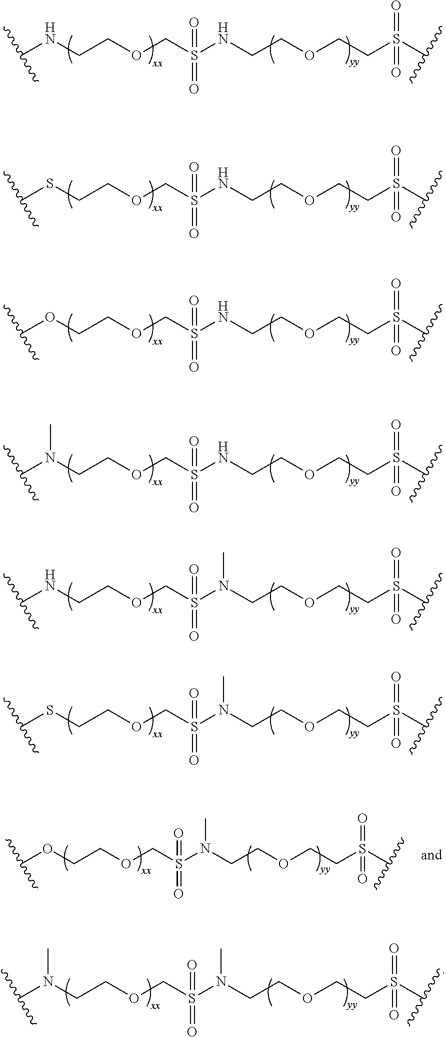

In certain embodiments Linker^{B} is selected from:

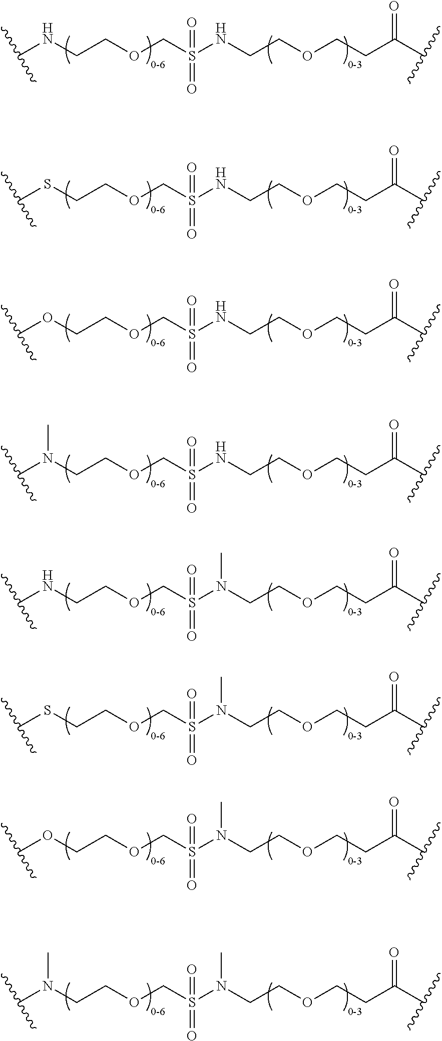

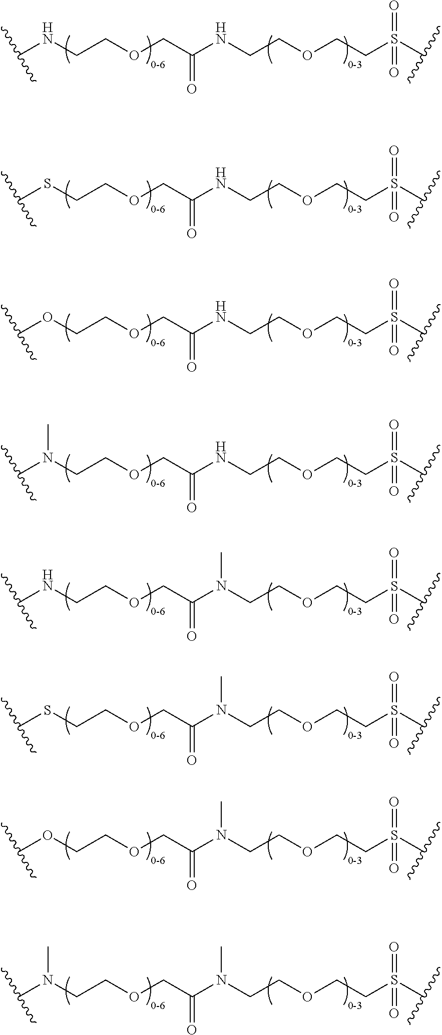

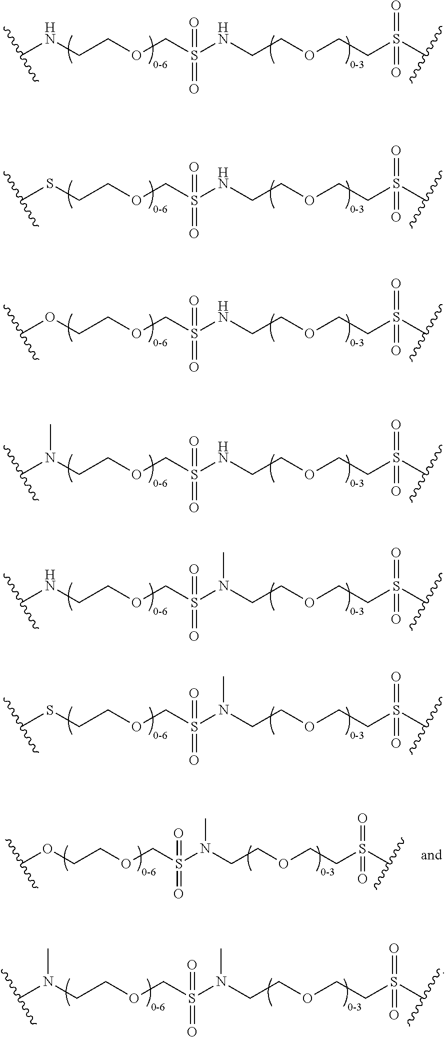

In certain embodiments Linker^{C} is selected from:

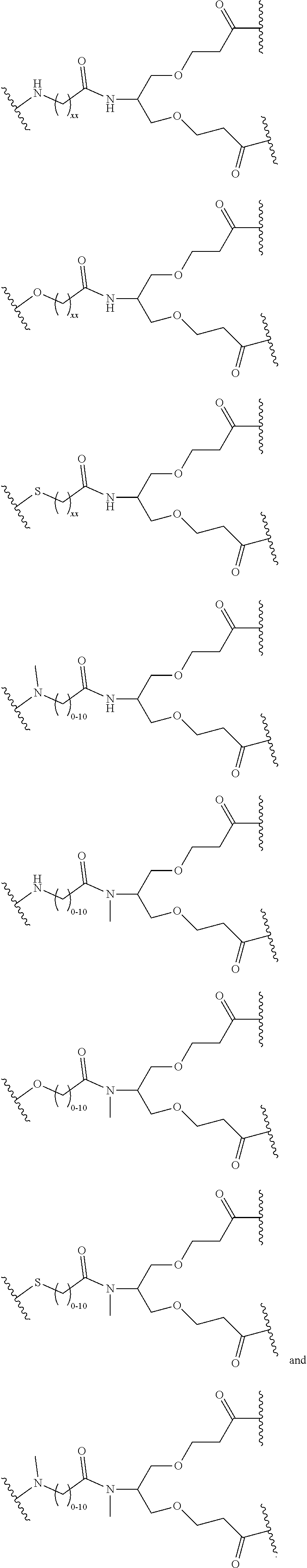

In certain embodiments Linker^{C} is selected from:

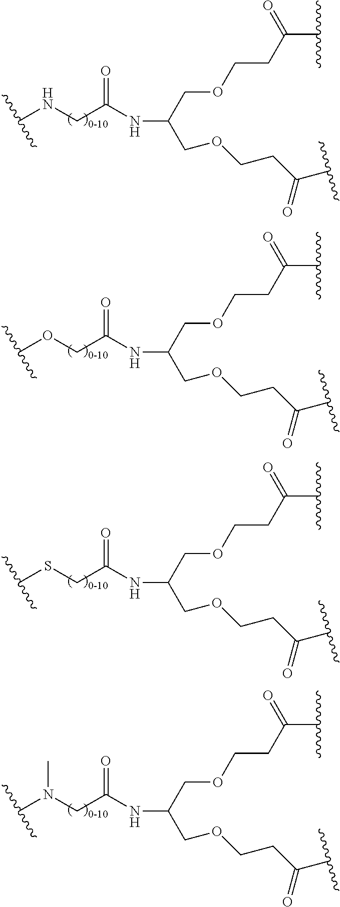

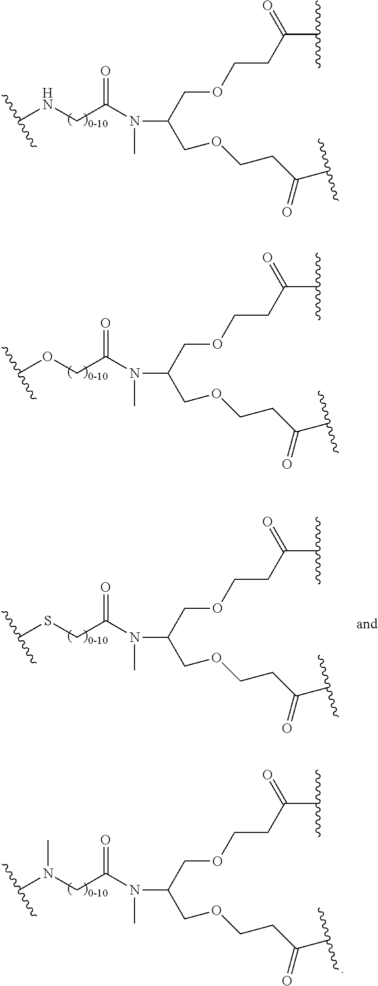

In certain embodiments Linker^{C} is selected from:

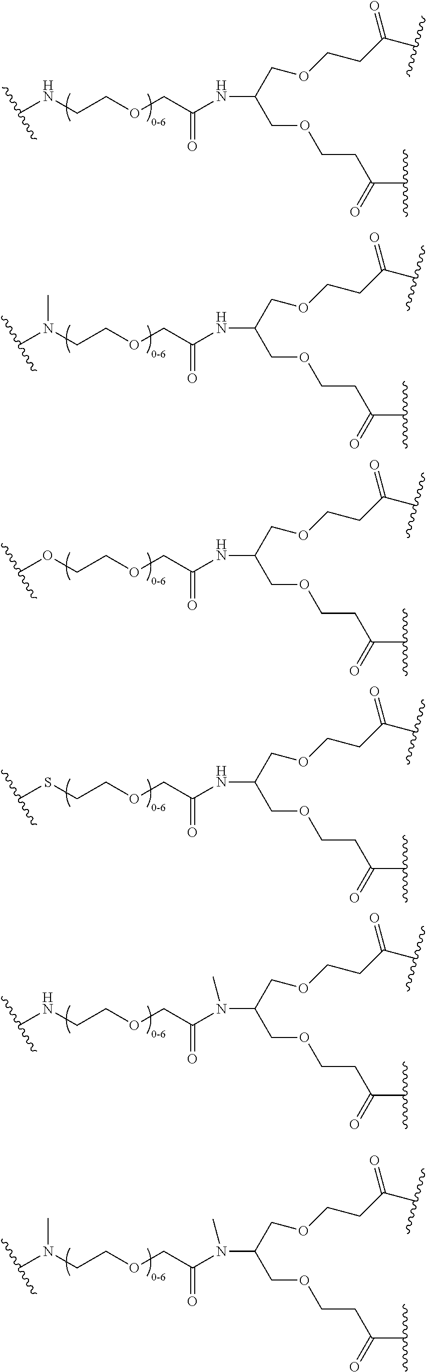

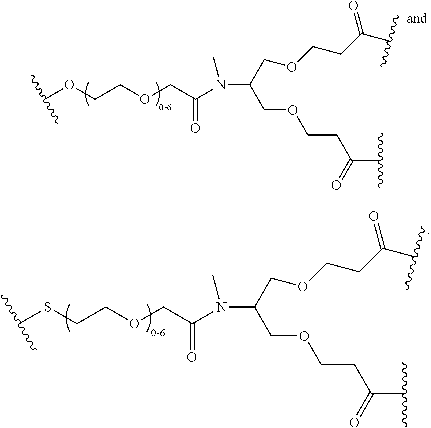

In certain embodiments Linker^{C} is selected from:

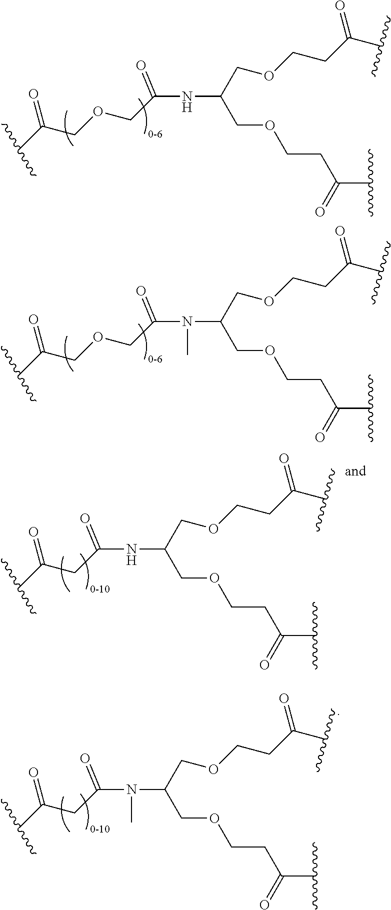

In certain embodiments Linker^{C} is selected from:

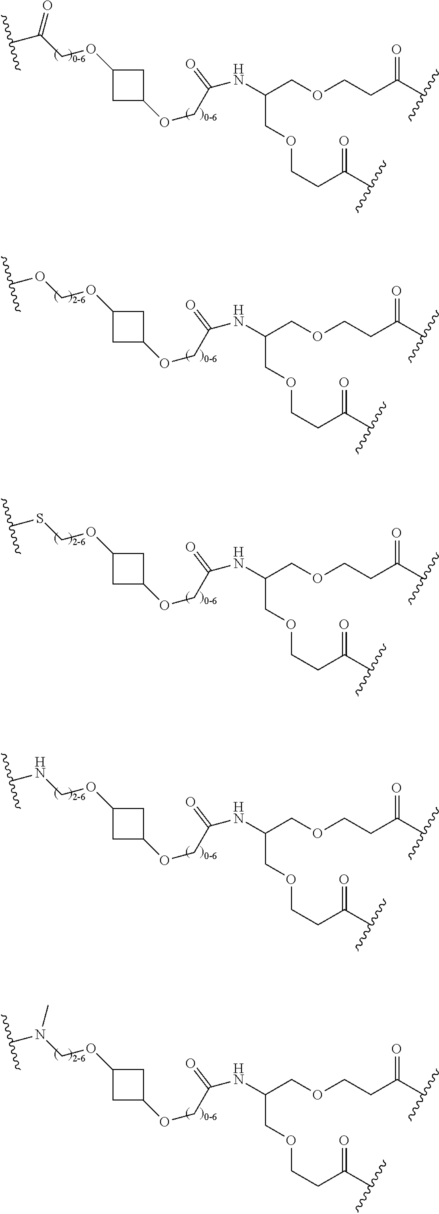

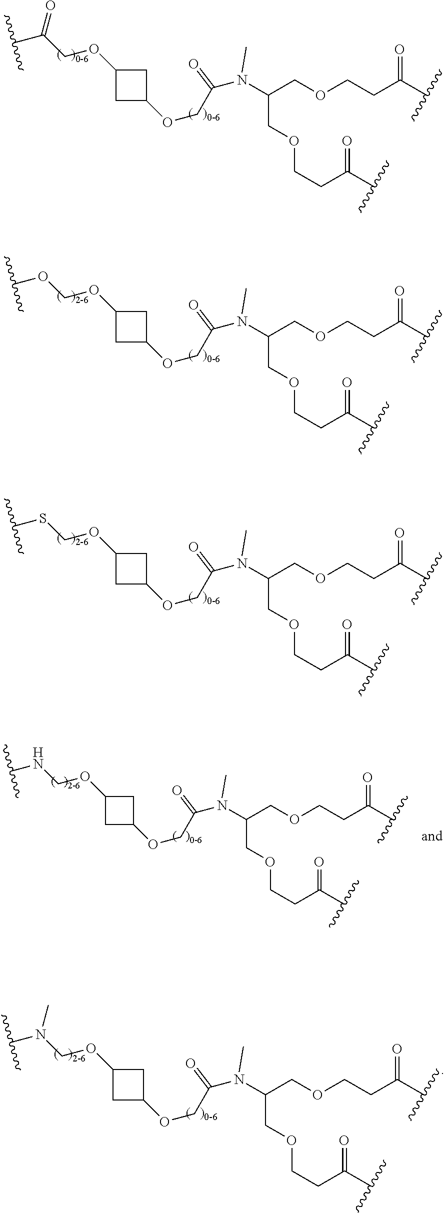

In certain embodiments Linker^{C} is selected from:

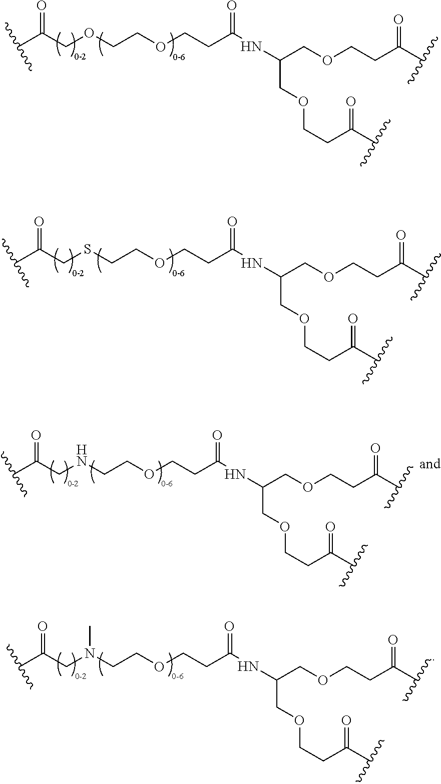

In certain embodiments Linker^{C} is selected from:

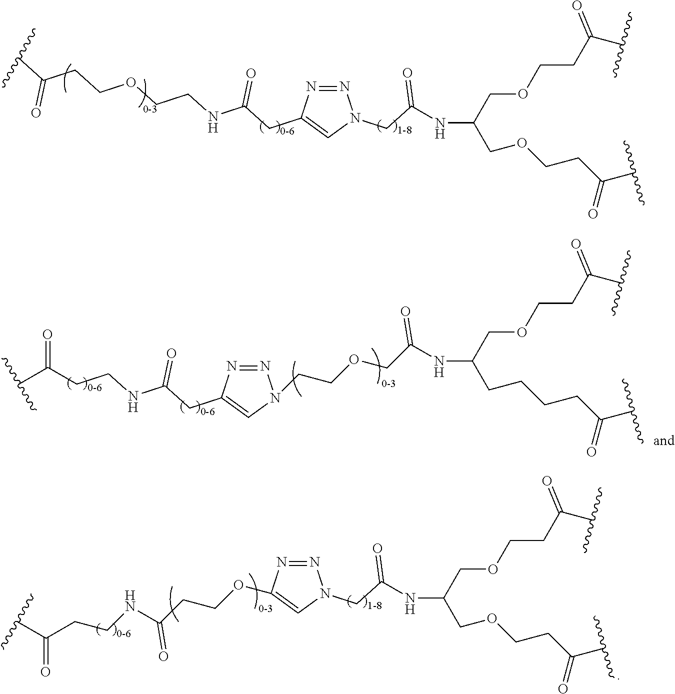

In certain embodiments Linker^{C} is selected from:

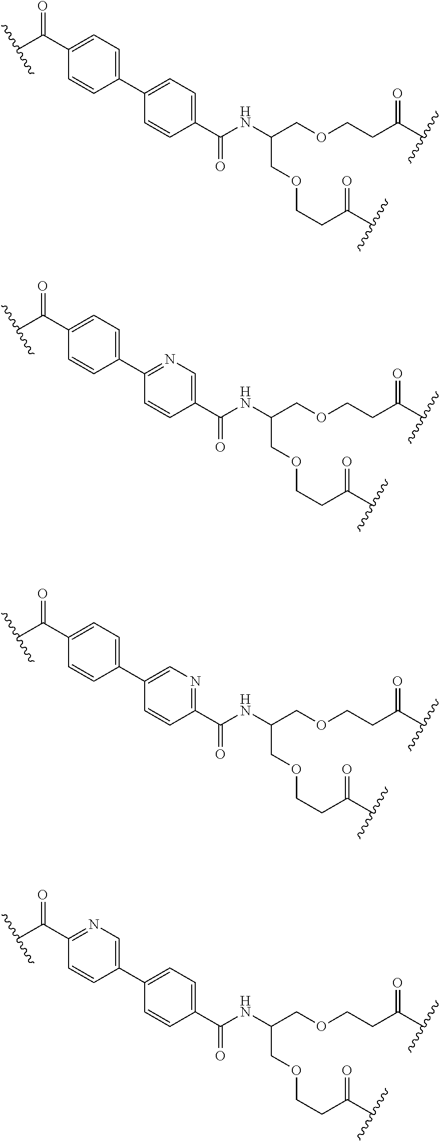 [SMILES: *C(=O)CCOCC(COCCC(*)=O)NC(=O)c1ccc(-c2ccc(C(*)=O)cc2)cc1 ; *C(=O)CCOCC(COCCC(*)=O)NC(=O)c1ccc(-c2ccc(C(*)=O)cc2)nc1 ; *C(=O)CCOCC(COCCC(*)=O)NC(=O)c1ccc(-c2ccc(C(*)=O)cc2)cn1 ; *C(=O)CCOCC(COCCC(*)=O)NC(=O)c1ccc(-c2ccc(C(*)=O)nc2)cc1]

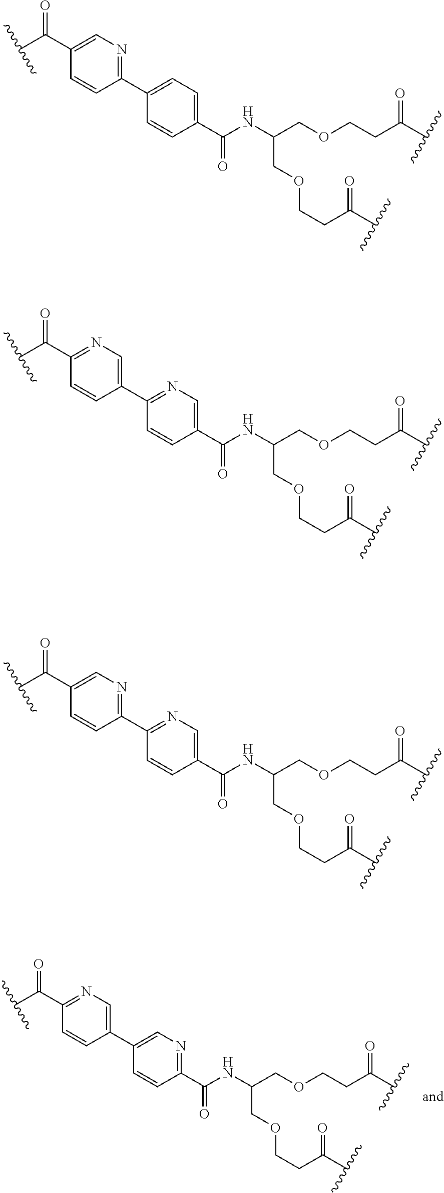 [SMILES: *C(=O)CCOCC(COCCC(*)=O)NC(=O)c1ccc(-c2ccc(C(*)=O)cn2)cc1 ; *C(=O)CCOCC(COCCC(*)=O)NC(=O)c1ccc(-c2ccc(C(*)=O)nc2)nc1 ; *C(=O)CCOCC(COCCC(*)=O)NC(=O)c1ccc(-c2ccc(C(*)=O)cn2)nc1 ; *C(=O)CCOCC(COCCC(*)=O)NC(=O)c1ccc(-c2ccc(C(*)=O)nc2)cn1]

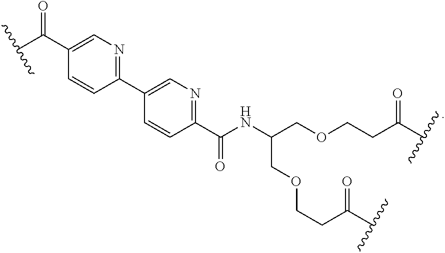 [SMILES: *C(=O)CCOCC(COCCC(*)=O)NC(=O)c1ccc(-c2ccc(C(*)=O)cn2)cn1]

In certain embodiments Linker^{D} is selected from:

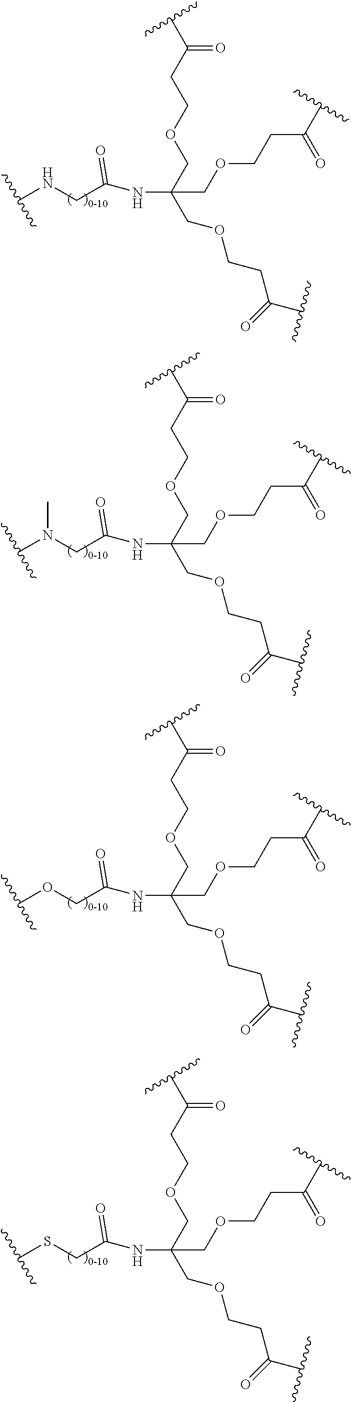

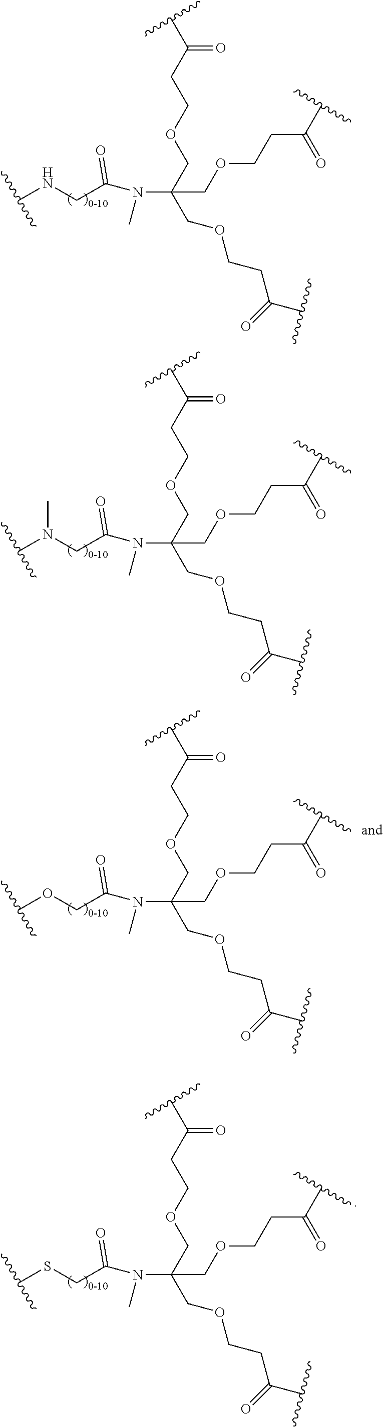

In certain embodiments Linker^{D} is selected from:

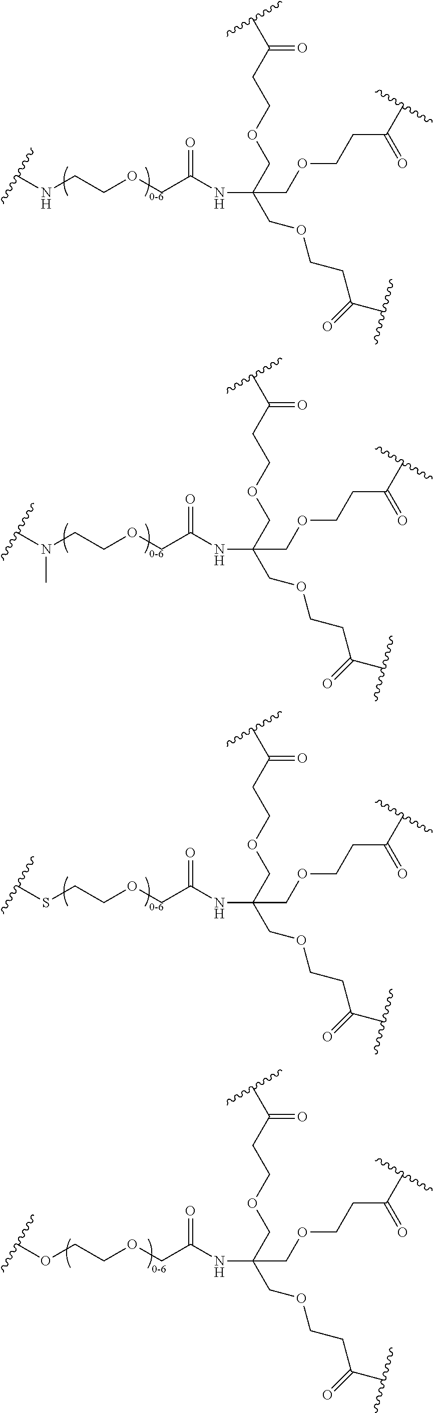

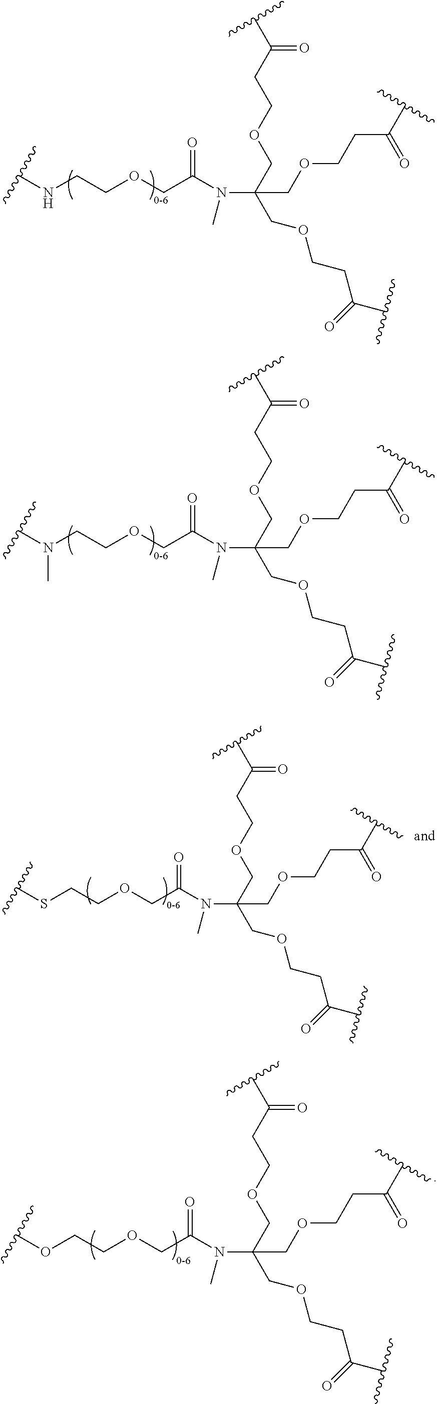

In certain embodiments Linker^{D} is selected from:

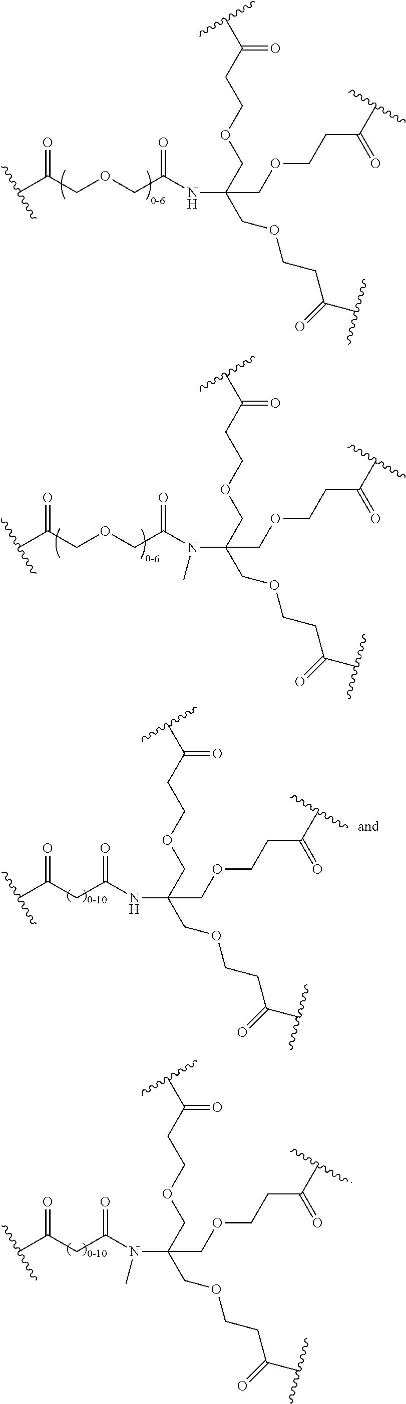

In certain embodiments Linker^{D} is selected from:

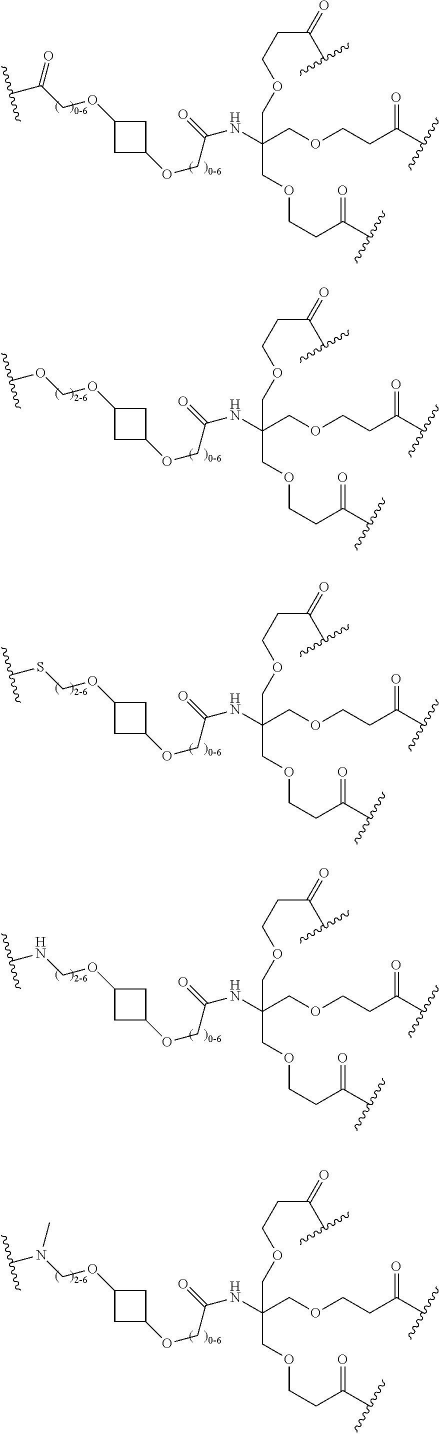

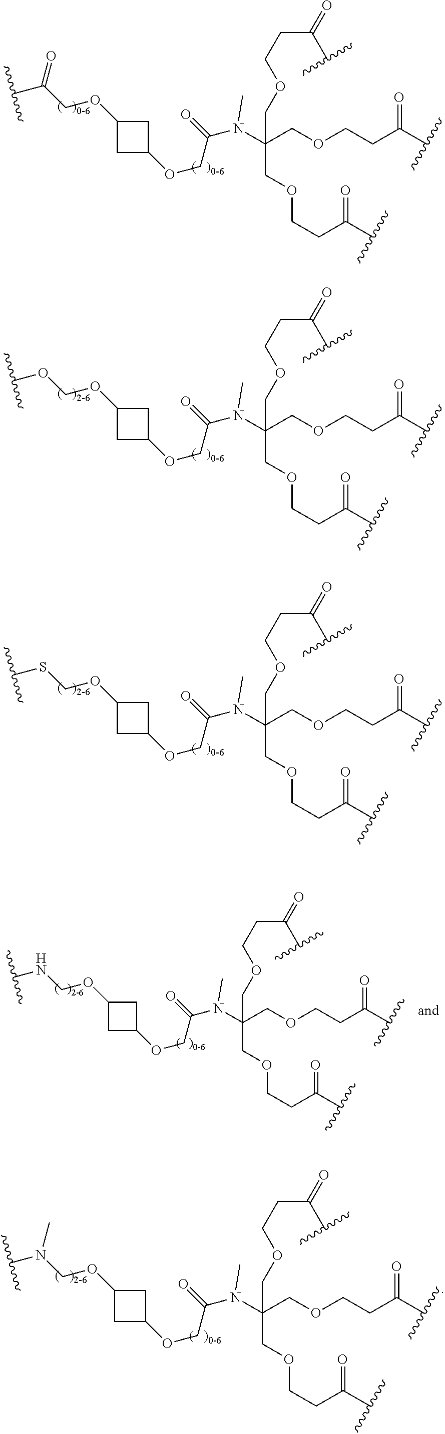

In certain embodiments Linker^{D} is selected from:

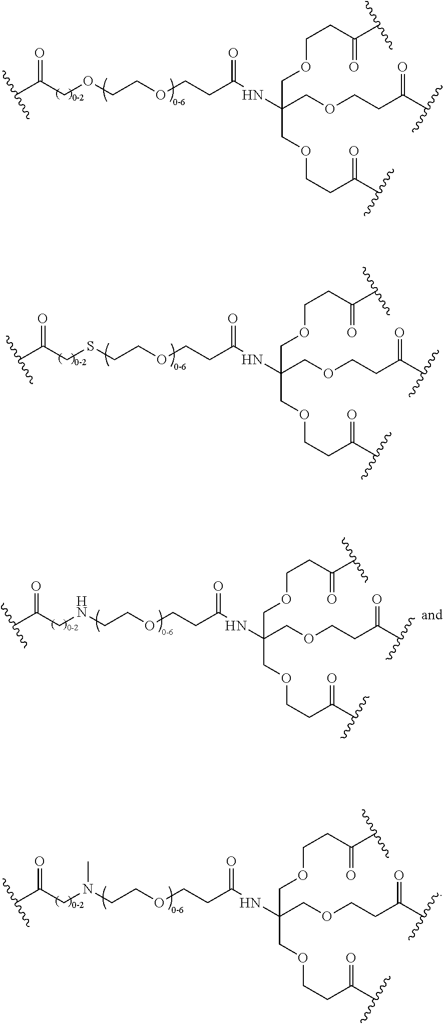

In certain embodiments Linker^{D} is selected from:

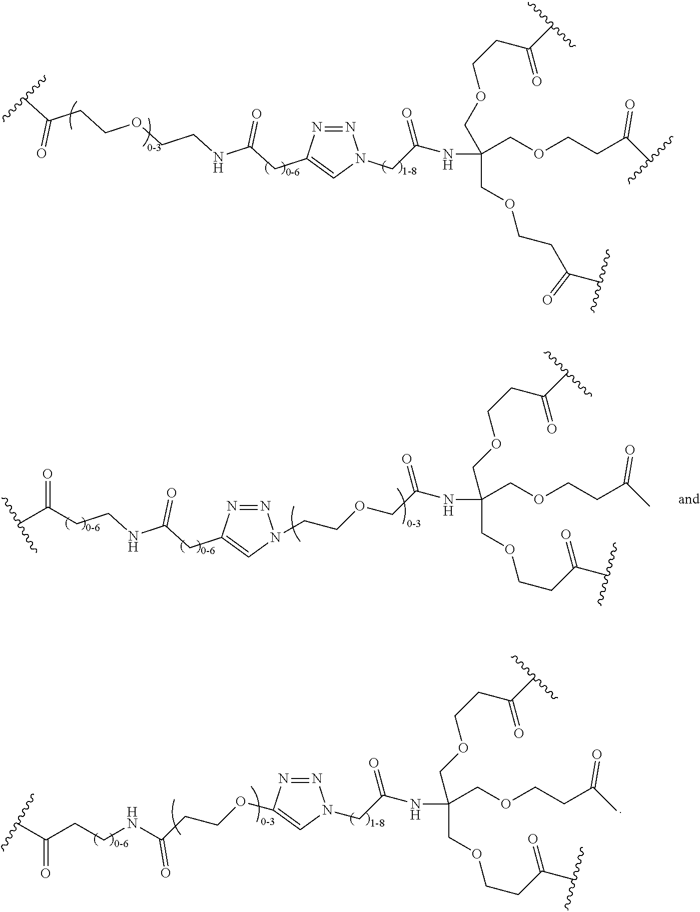

In certain embodiments Linker^{D} is selected from:

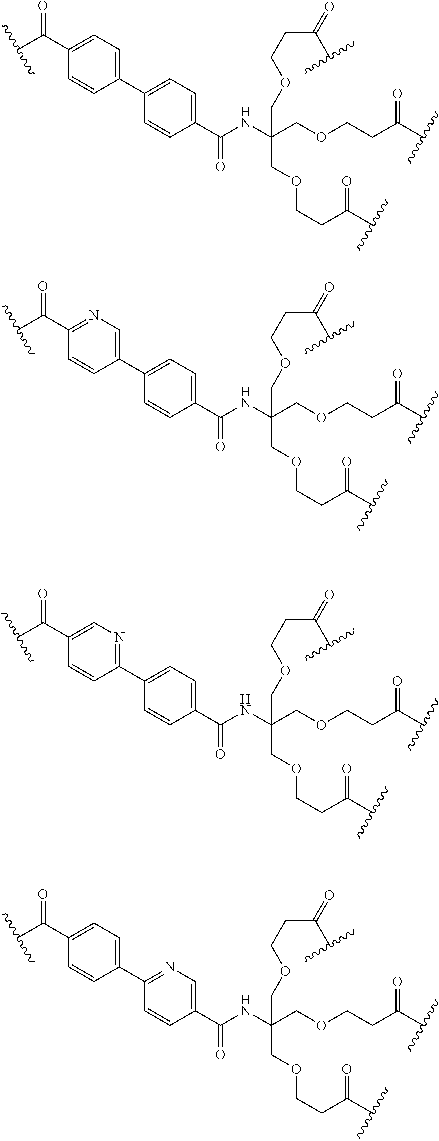 [SMILES: *C(=O)CCOCC(COCCC(*)=O)(COCCC(*)=O)NC(=O)c1ccc(-c2ccc(C(*)=O)cc2)cc1 ; *C(=O)CCOCC(COCCC(*)=O)(COCCC(*)=O)NC(=O)c1ccc(-c2ccc(C(*)=O)nc2)cc1 ; *C(=O)CCOCC(COCCC(*)=O)(COCCC(*)=O)NC(=O)c1ccc(-c2ccc(C(*)=O)cn2)cc1 ; *C(=O)CCOCC(COCCC(*)=O)(COCCC(*)=O)NC(=O)c1ccc(-c2ccc(C(*)=O)cc2)nc1]

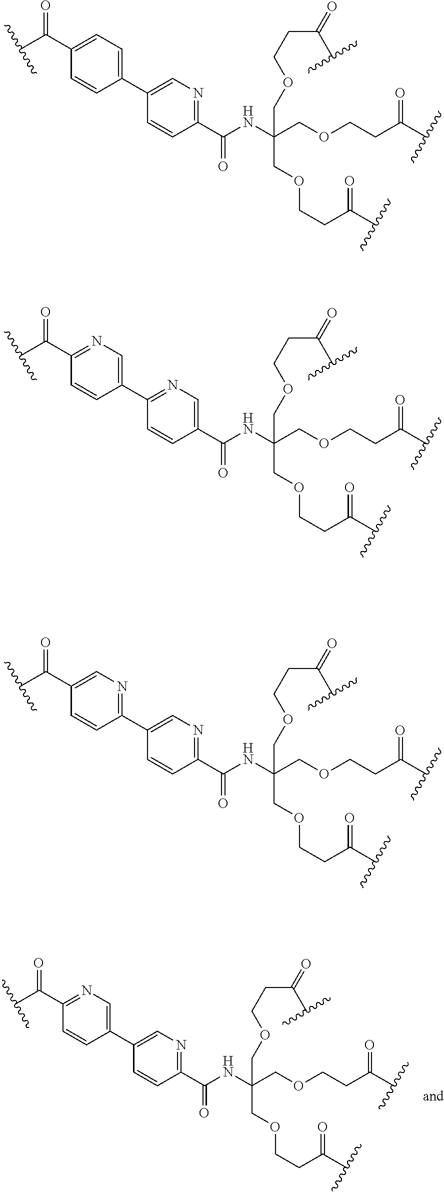 [SMILES: *C(=O)CCOCC(COCCC(*)=O)(COCCC(*)=O)NC(=O)c1ccc(-c2ccc(C(*)=O)cc2)cn1 ; *C(=O)CCOCC(COCCC(*)=O)(COCCC(*)=O)NC(=O)c1ccc(-c2ccc(C(*)=O)nc2)nc1 ; *C(=O)CCOCC(COCCC(*)=O)(COCCC(*)=O)NC(=O)c1ccc(-c2ccc(C(*)=O)cn2)cn1 ; *C(=O)CCOCC(COCCC(*)=O)(COCCC(*)=O)NC(=O)c1ccc(-c2ccc(C(*)=O)nc2)cn1]

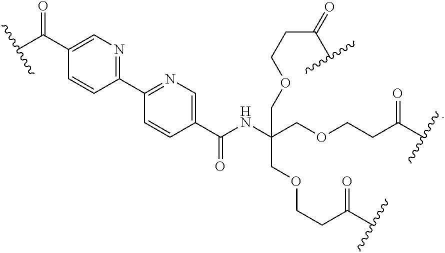 [SMILES: *C(=O)CCOCC(COCCC(*)=O)(COCCC(*)=O)NC(=O)c1ccc(-c2ccc(C(*)=O)cn2)nc1]

In certain embodiments, the Linker^{A} is selected from

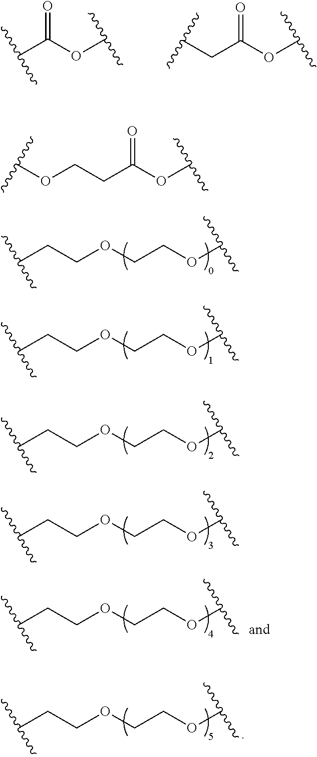

In certain embodiments, the Linker^{A} is selected from

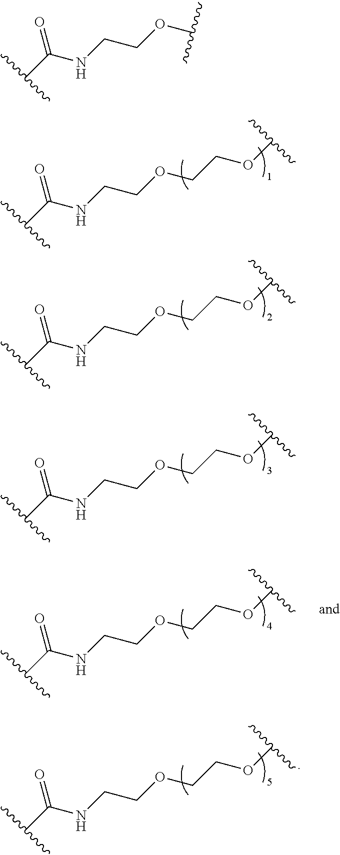

In certain embodiments, the Linker^{A} is selected from

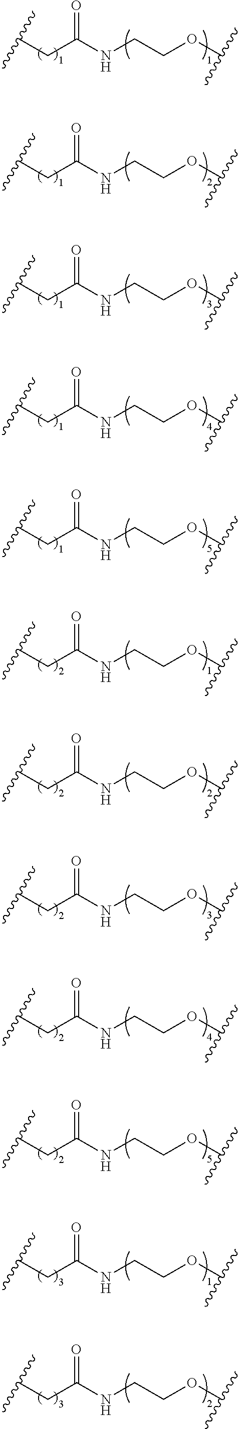

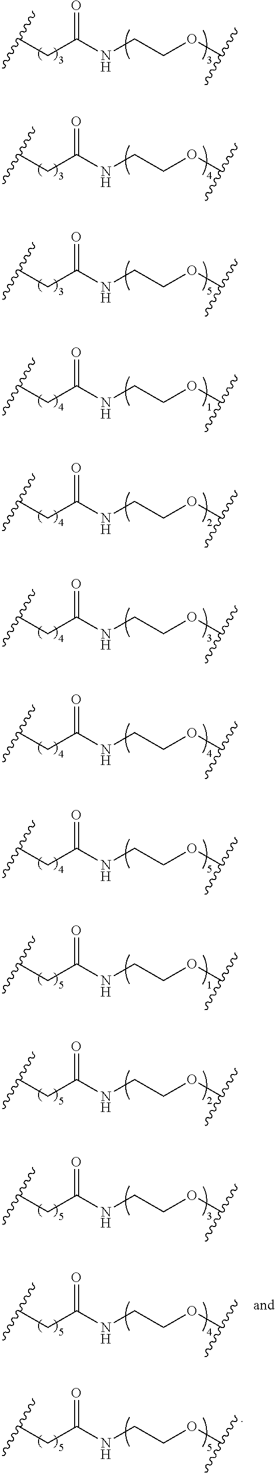

In certain embodiments, the Linker^{A} is selected from

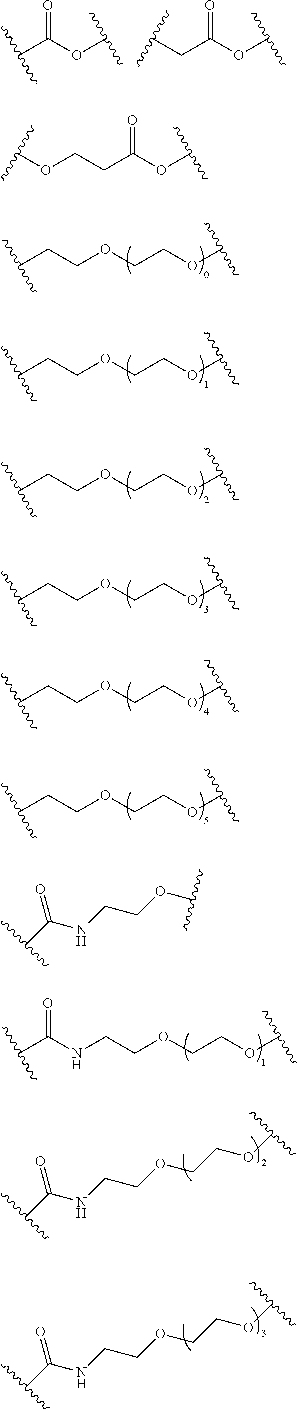

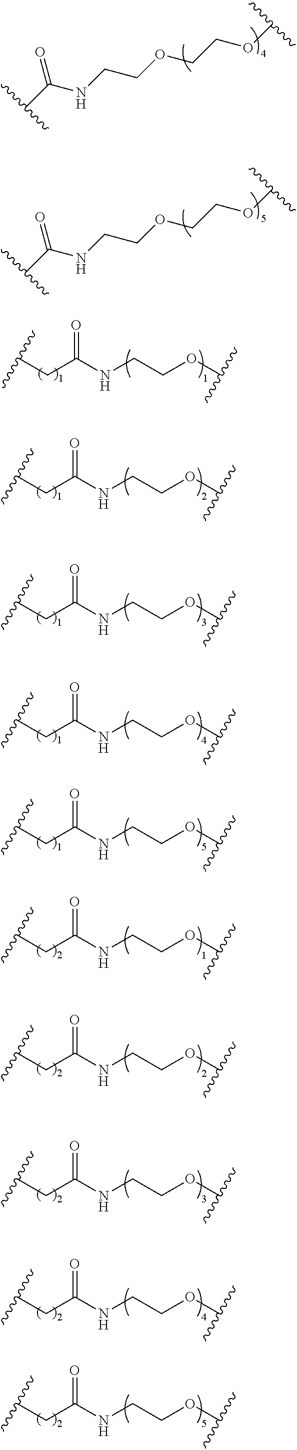

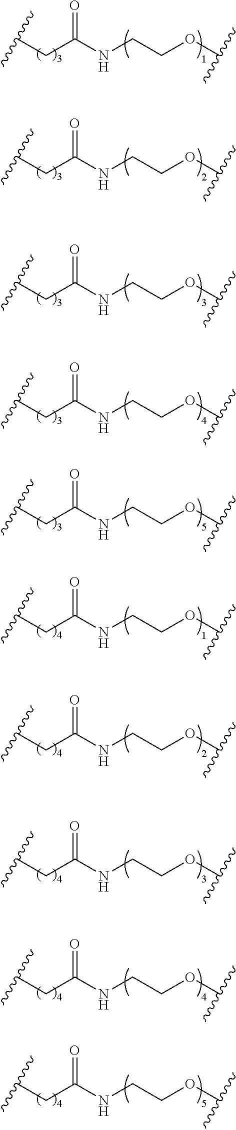

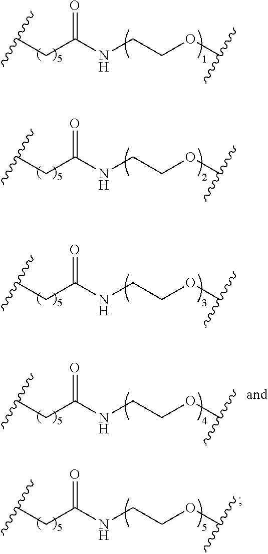

wherein each is optionally substituted with 1, 2, 3, or 4 substituents substituent selected from R²¹.

In certain embodiments Linker^{A} is selected from:

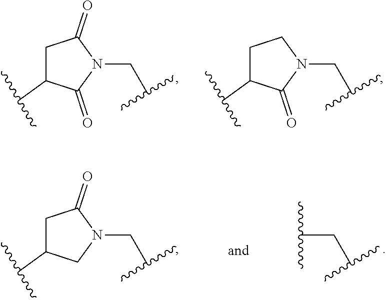

In certain embodiments Linker^{A} is selected from:

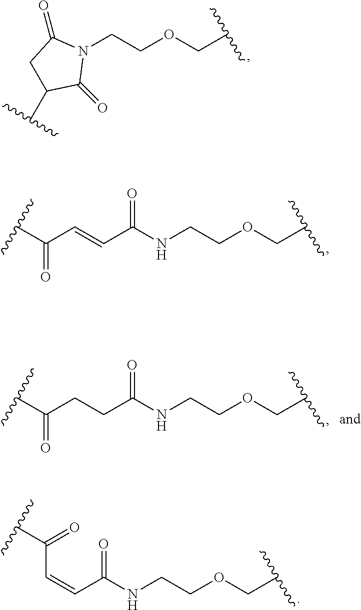 [SMILES: *COCCN1C(=O)CC(*)C1=O ; *COCCNC(=O)/C=C/C(*)=O ; *COCCNC(=O)CCC(*)=O ; *COCCNC(=O)/C=C\C(*)=O]

In certain embodiments Linker^{A} is selected from:

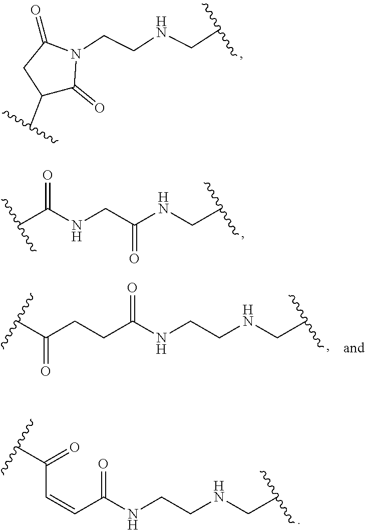

In certain embodiments Linker^{A} is selected from:

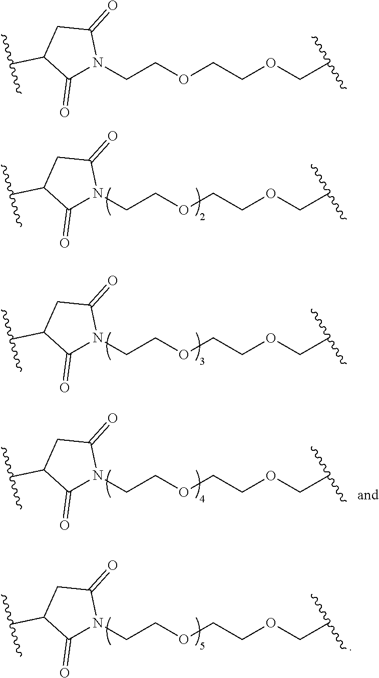

In certain embodiments Linker^{A} is selected from:

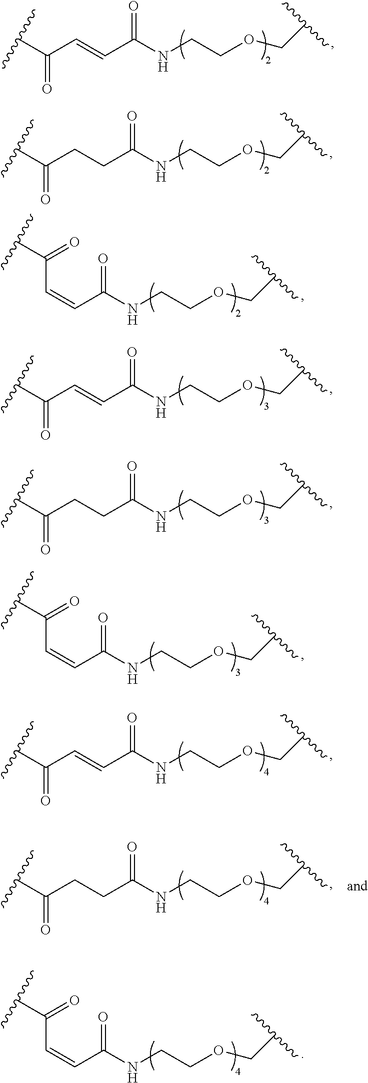

In certain embodiments Linker^{A} is selected from:

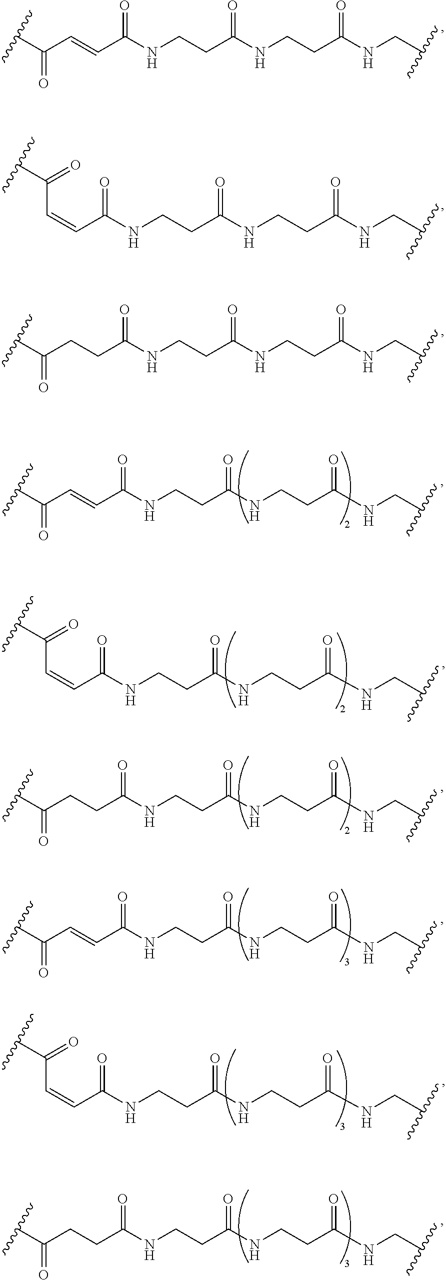

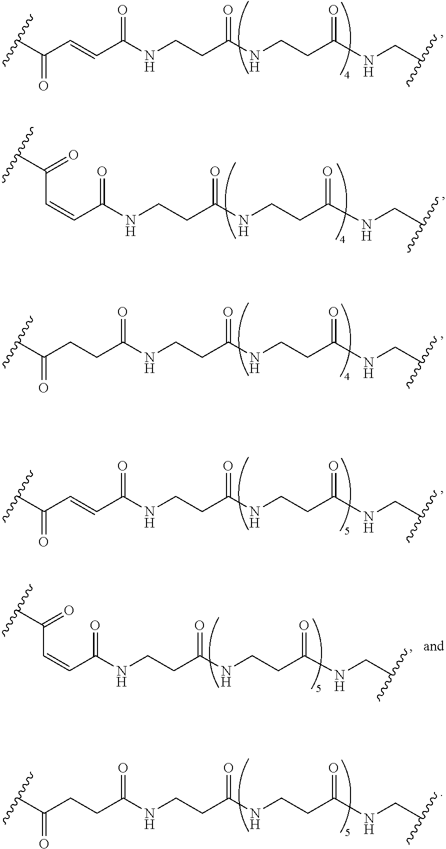

In certain embodiments Linker^{A} is selected from:

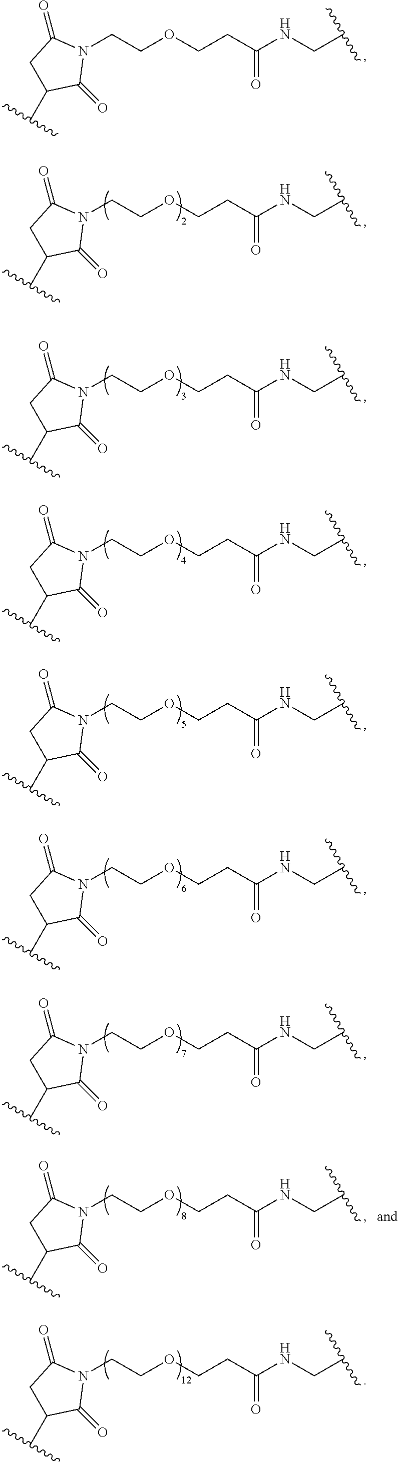

In certain embodiments Linker^{A} is selected from:

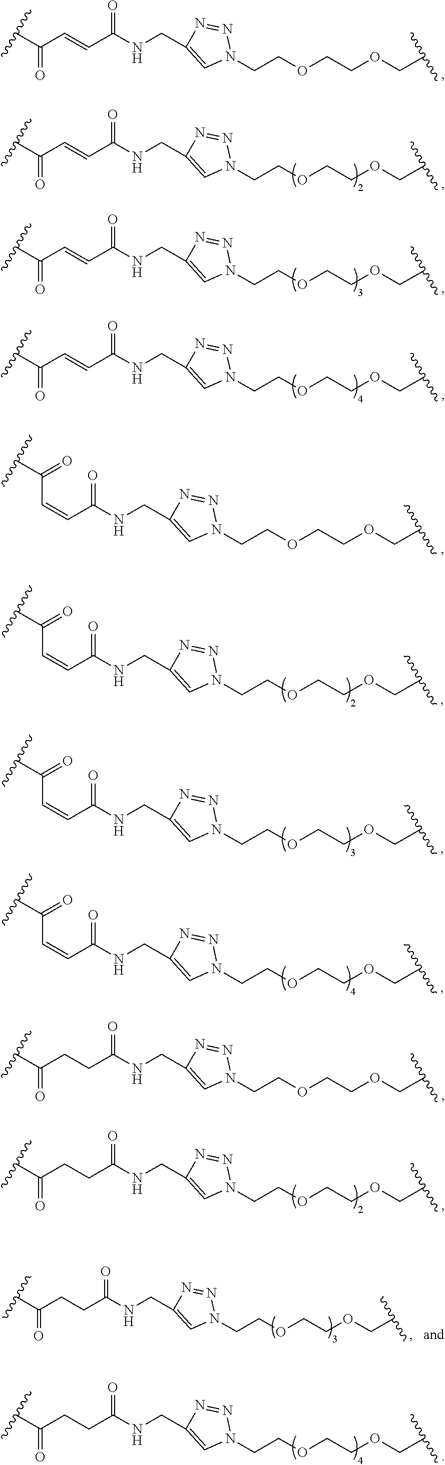

In certain embodiments Linker^{A} is selected from:

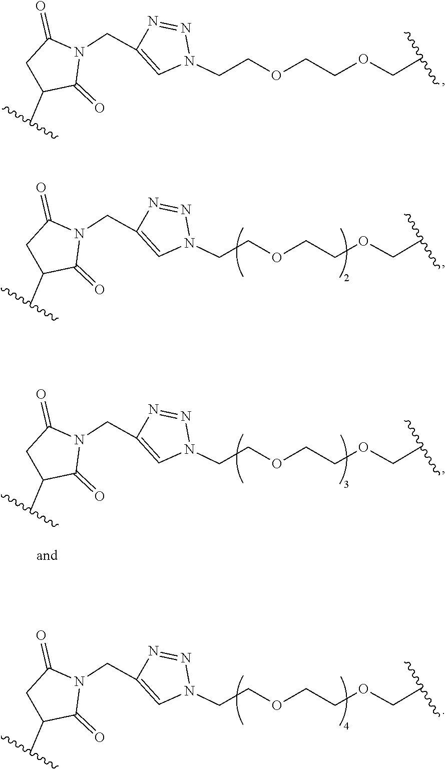

In certain embodiments Linker^{A} is selected from:

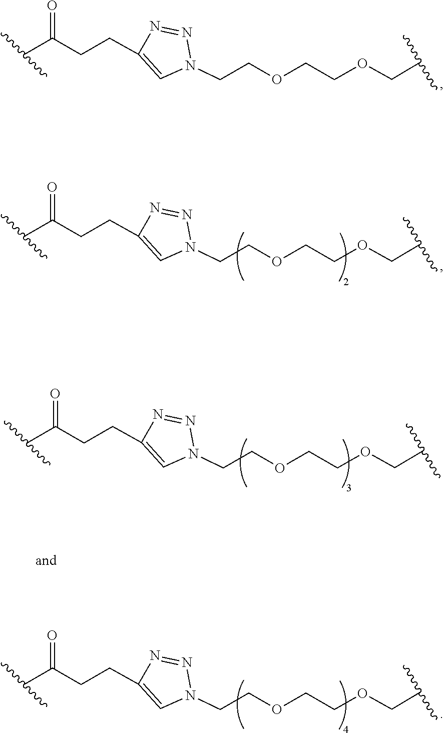

In certain embodiments Linker^{A} is selected from:

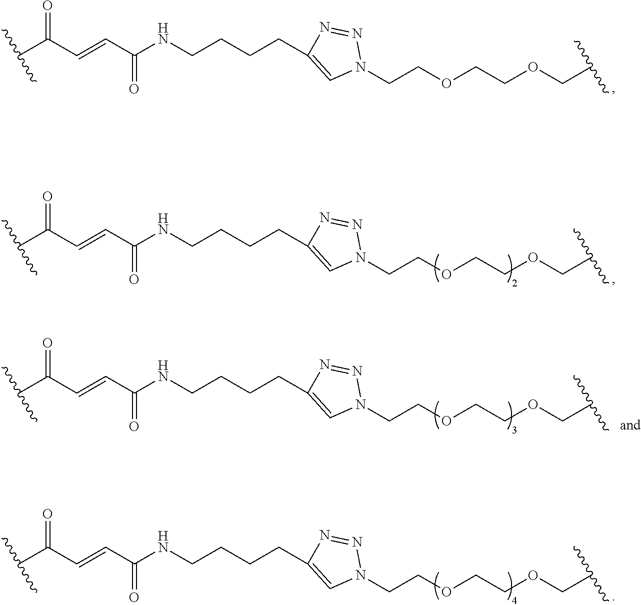

In certain embodiments Linker^{A} is selected from:

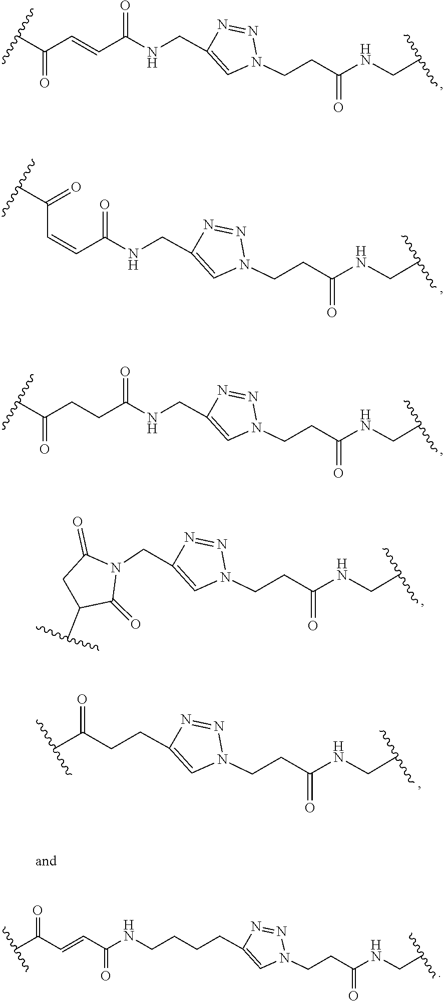 [SMILES: *CNC(=O)CCn1cc(CNC(=O)/C=C/C(*)=O)nn1 ; *CNC(=O)CCn1cc(CNC(=O)/C=C\C(*)=O)nn1 ; *CNC(=O)CCn1cc(CNC(=O)CCC(*)=O)nn1 ; *CNC(=O)CCn1cc(CN2C(=O)CC(*)C2=O)nn1 ; *CNC(=O)CCn1cc(CCC(*)=O)nn1 ; *CNC(=O)CCn1cc(CCCCNC(=O)/C=C/C(*)=O)nn1]

In certain embodiments Linker^{A} is selected from:

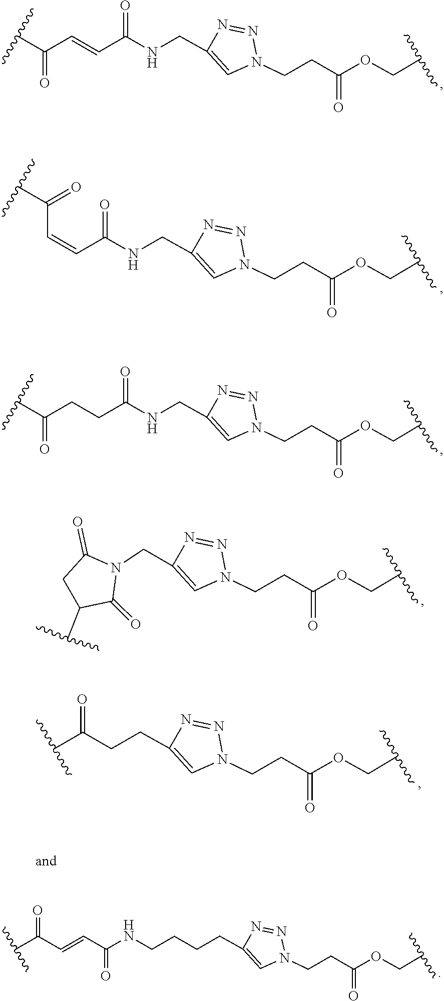 [SMILES: *COC(=O)CCn1cc(CNC(=O)/C=C/C(*)=O)nn1 ; *COC(=O)CCn1cc(CNC(=O)/C=C\C(*)=O)nn1 ; *COC(=O)CCn1cc(CNC(=O)CCC(*)=O)nn1 ; *COC(=O)CCn1cc(CN2C(=O)CC(*)C2=O)nn1 ; *COC(=O)CCn1cc(CCC(*)=O)nn1 ; *COC(=O)CCn1cc(CCCCNC(=O)/C=C/C(*)=O)nn1]

In certain embodiments, the Linker^{B} is selected from

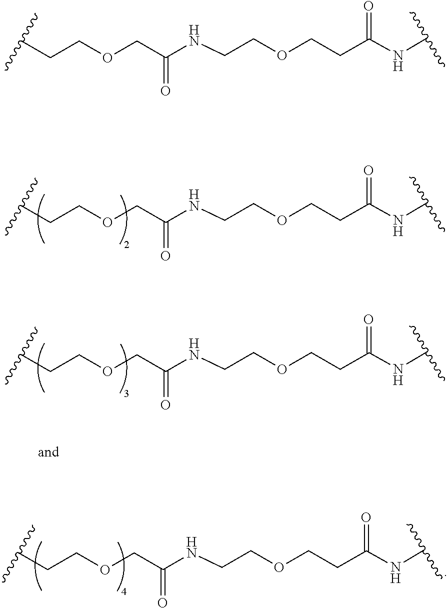

In certain embodiments, the Linker^{B} is selected from

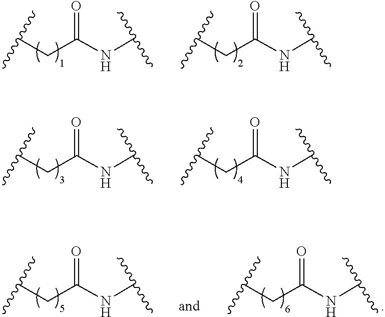

In certain embodiments, the Linker^{B} is selected from

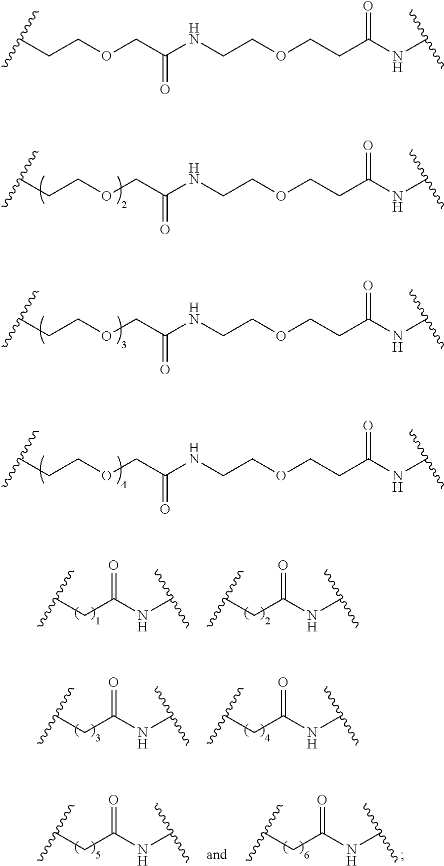

wherein each is optionally substituted with 1, 2, 3, or 4 substituents substituent selected from R²¹.

In certain embodiments Linker^{B} is selected from:

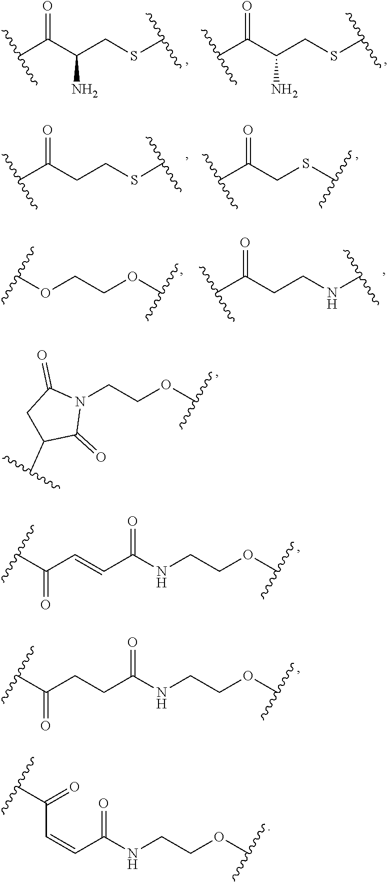

In certain embodiments Linker^{B} is selected from:

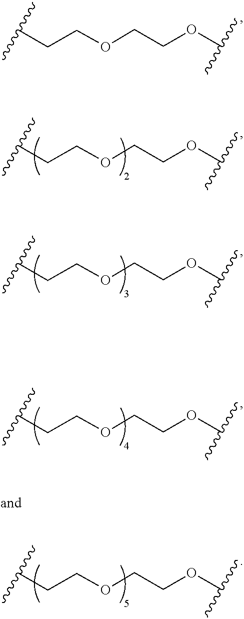

In certain embodiments Linker^{B} is selected from:

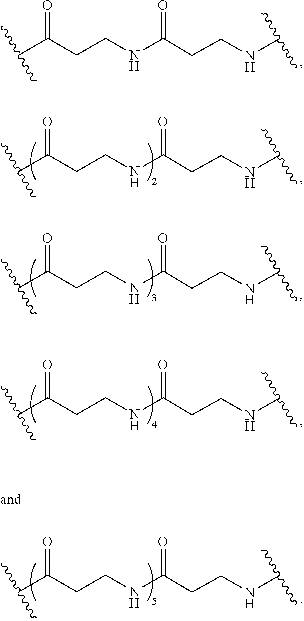

In certain embodiments Linker^{B} is selected from:

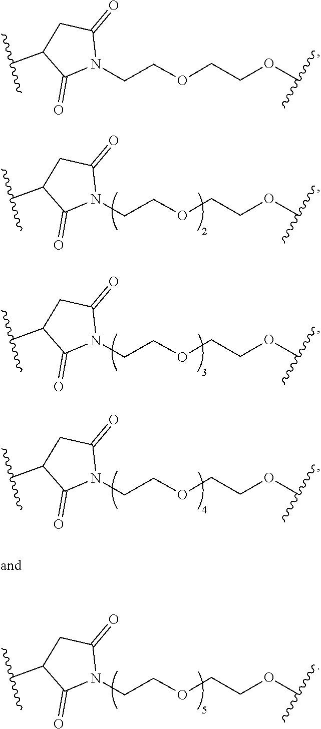

In certain embodiments Linker^{B} is selected from:

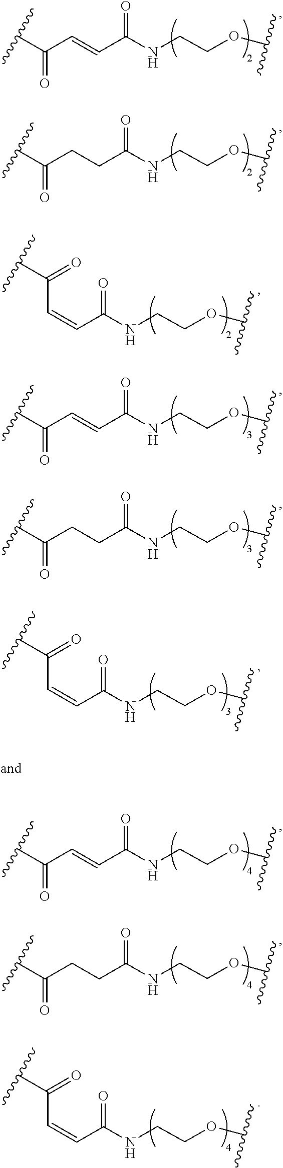

In certain embodiments Linker^{B} is selected from:

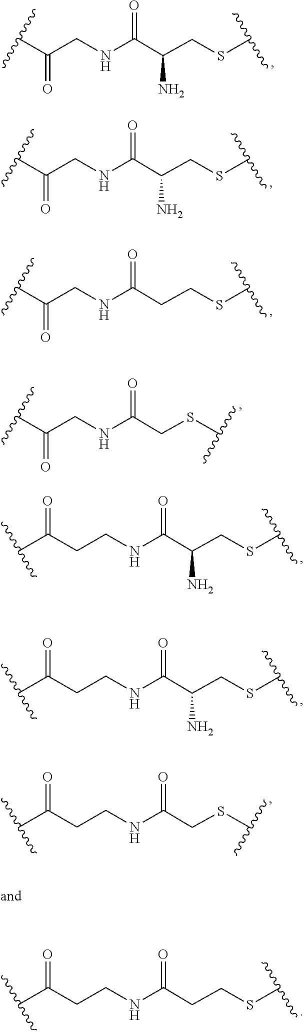

In certain embodiments Linker^{B} is selected from:

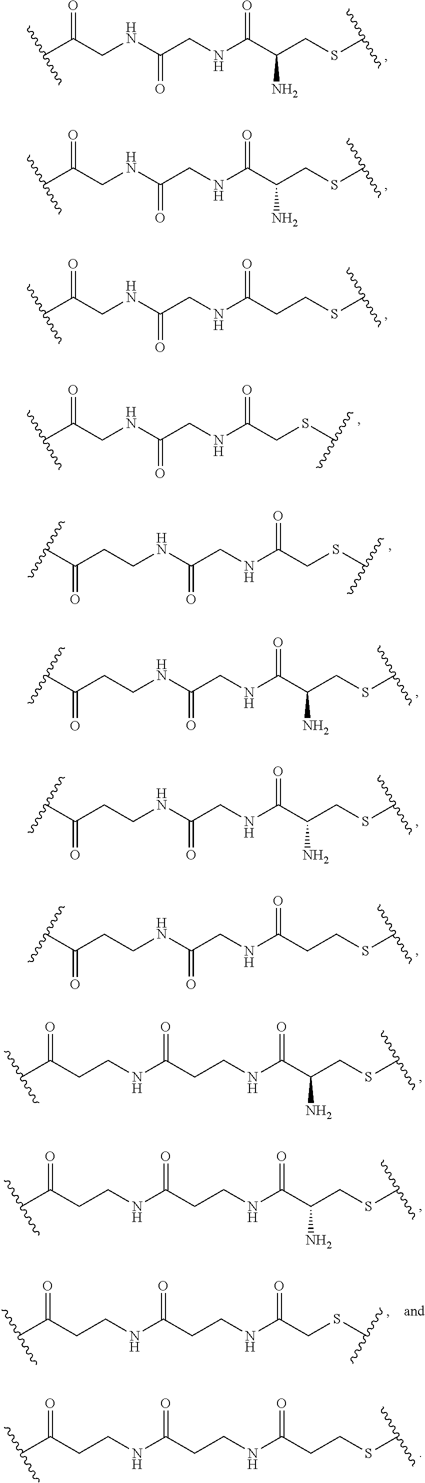

In certain embodiments Linker^{B} is selected from:

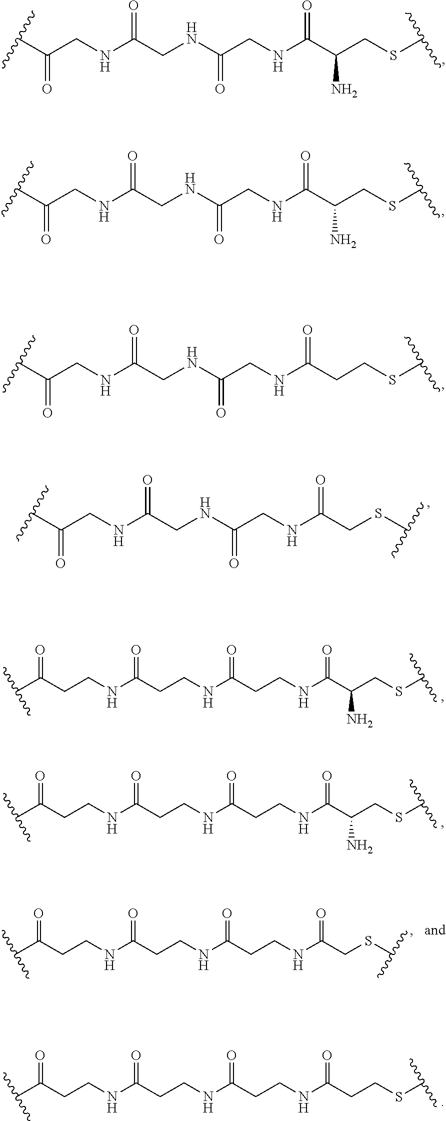

In certain embodiments Linker^{B}-Linker^{A} is selected from:

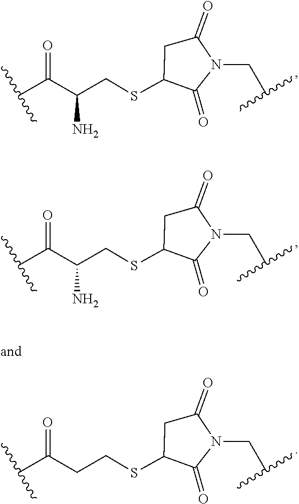 [SMILES: *CN1C(=O)CC(SC[C@@H](N)C(*)=O)C1=O ; *CN1C(=O)CC(SC[C@H](N)C(*)=O)C1=O ; *CN1C(=O)CC(SCCC(*)=O)C1=O]

In certain embodiments Linker^{B}-Linker^{A} is selected from:

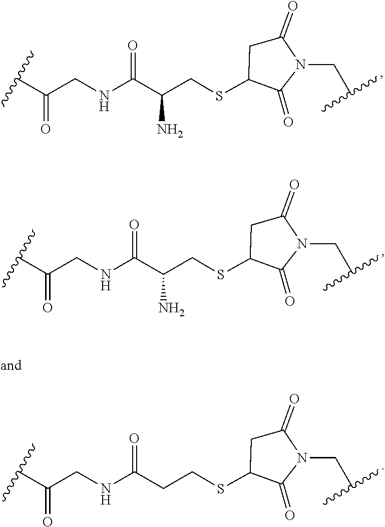 [SMILES: *CN1C(=O)CC(SC[C@@H](N)C(=O)NCC(*)=O)C1=O ; *CN1C(=O)CC(SC[C@H](N)C(=O)NCC(*)=O)C1=O ; *CN1C(=O)CC(SCCC(=O)NCC(*)=O)C1=O]

In certain embodiments, the Linker^{C} is selected from

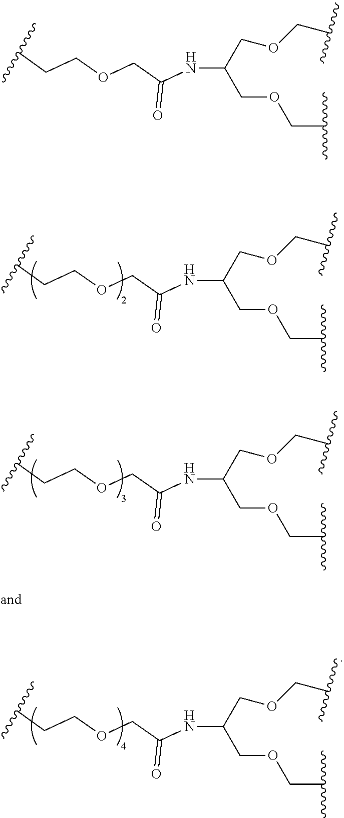

In certain embodiments, the Linker^{C} is selected from

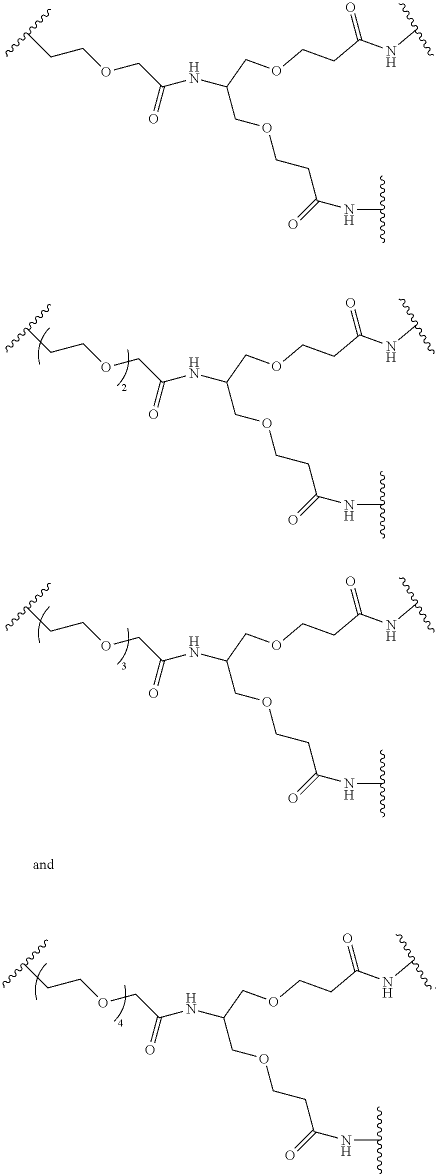

In certain embodiments, the Linker^{C} is selected from

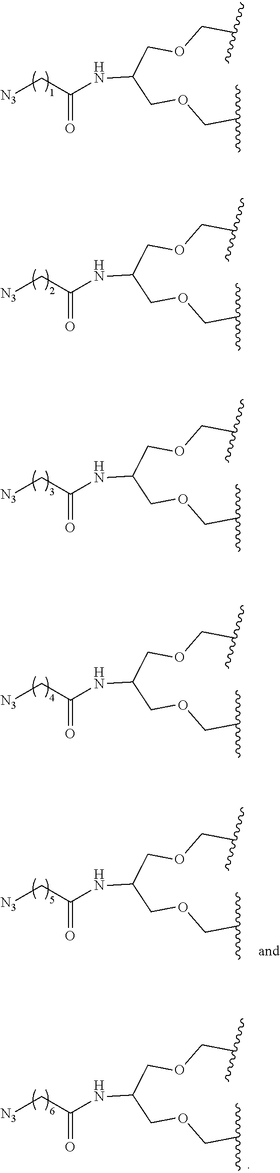

In certain embodiments, the Linker^{C} is selected from

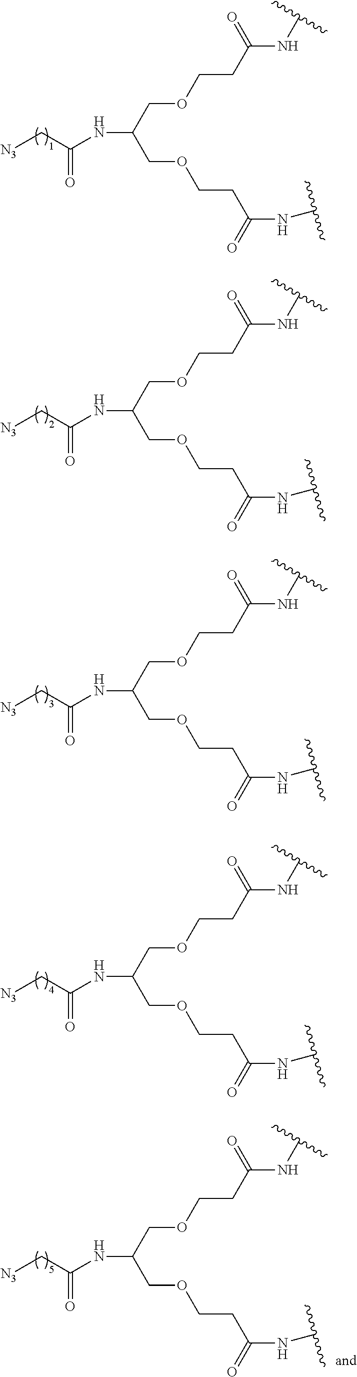

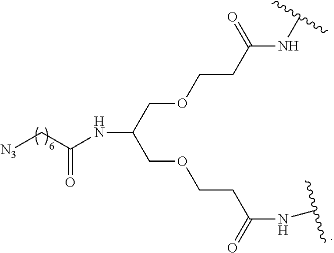

In certain embodiments, the Linker^{C} is selected from

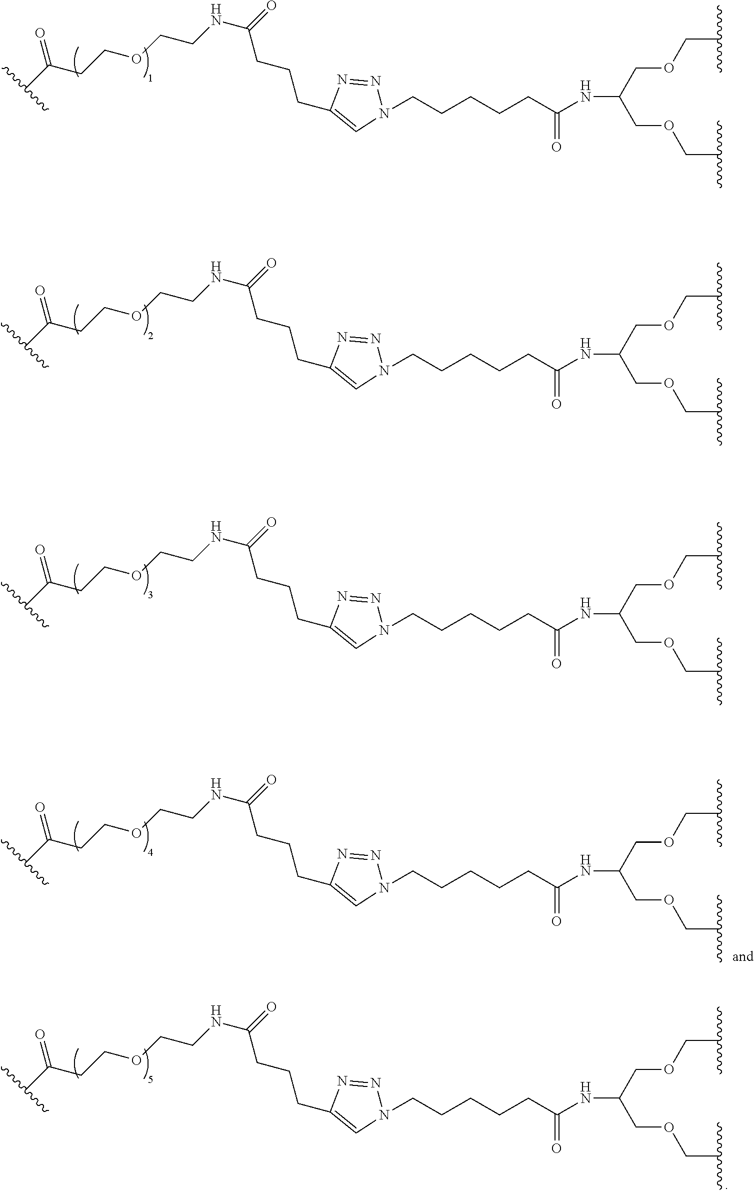

In certain embodiments, the Linker^{C} is selected from

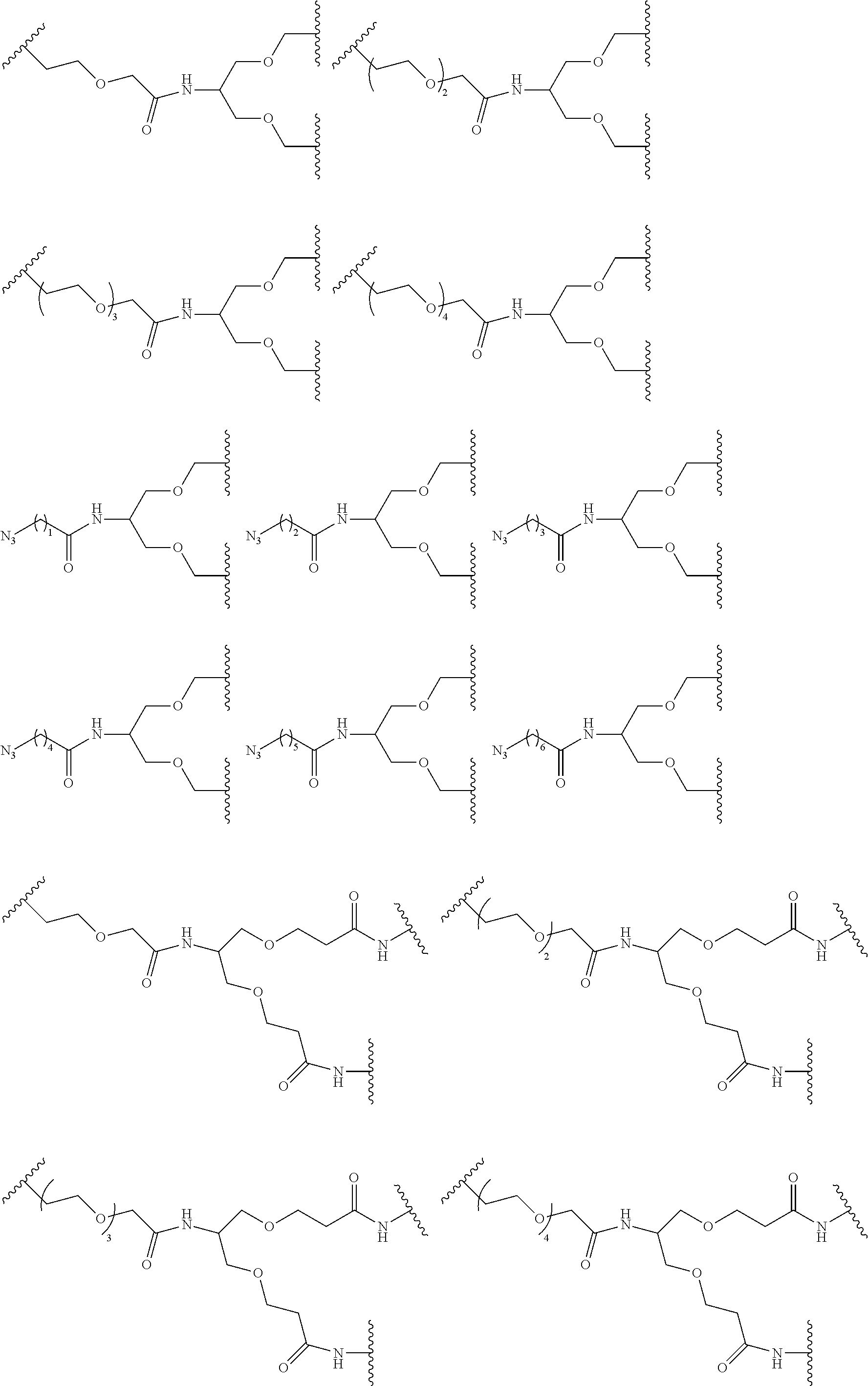

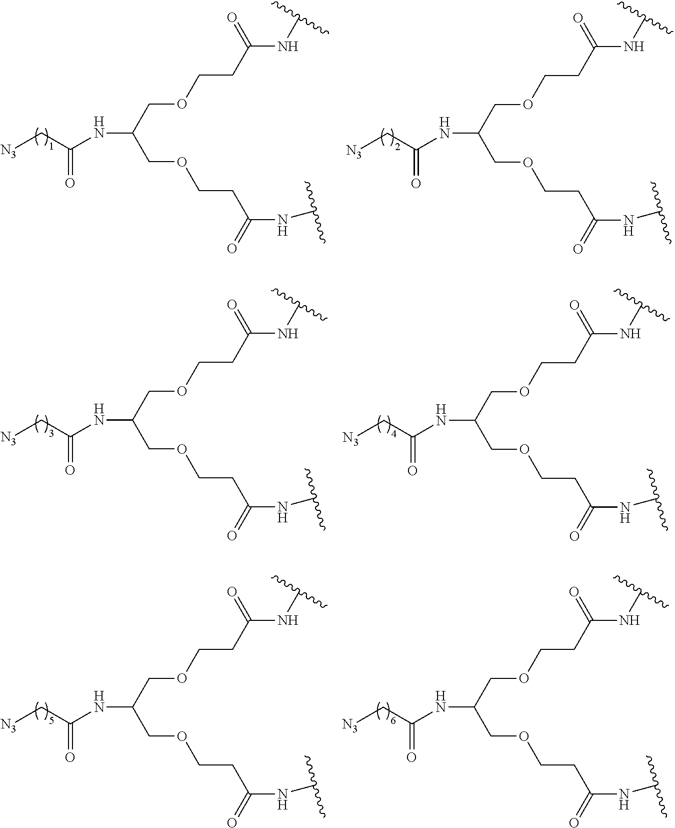

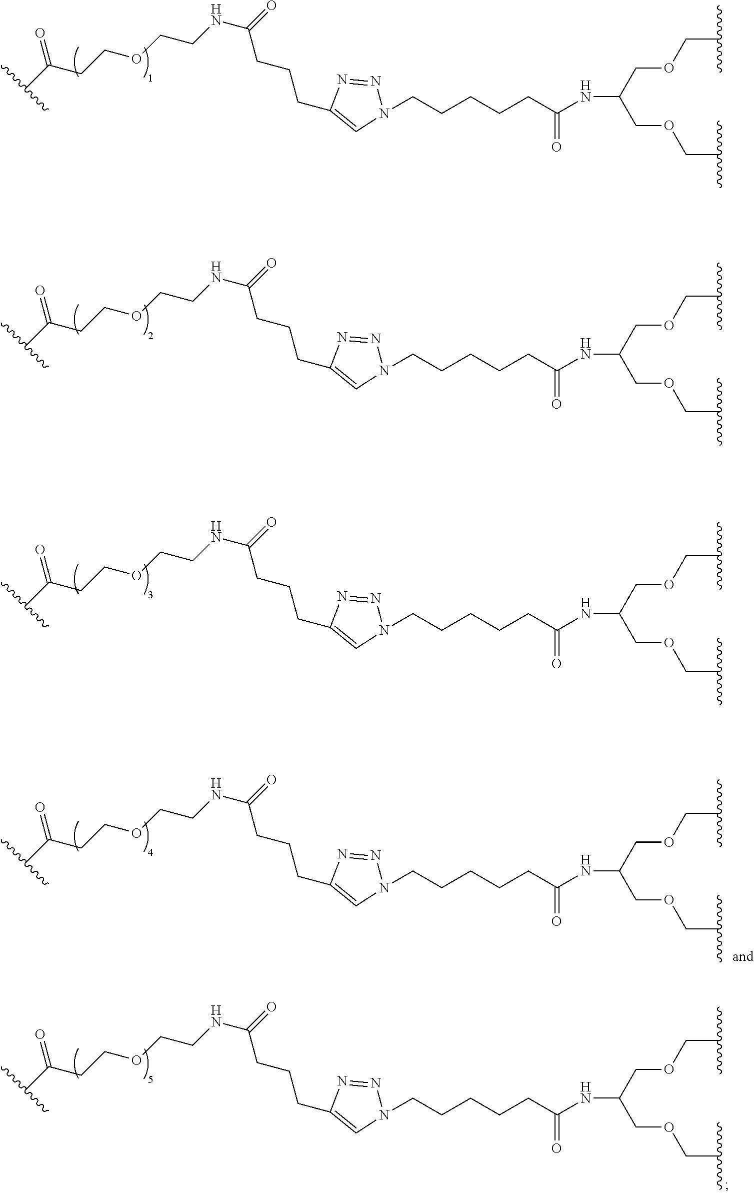

wherein each is optionally substituted with 1, 2, 3, or 4 substituents substituent selected from R²¹.

In certain embodiments Linker^{C} is selected from:

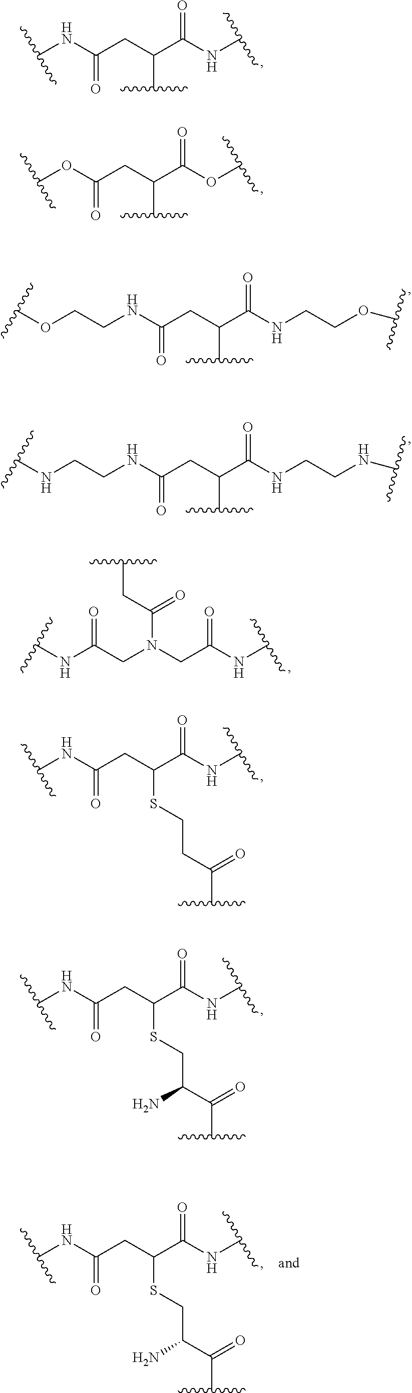

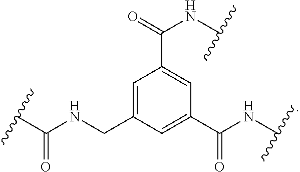 [SMILES: *NC(=O)c1cc(CNC(*)=O)cc(C(=O)N*)c1]

In certain embodiments Linker^{C} is selected from:

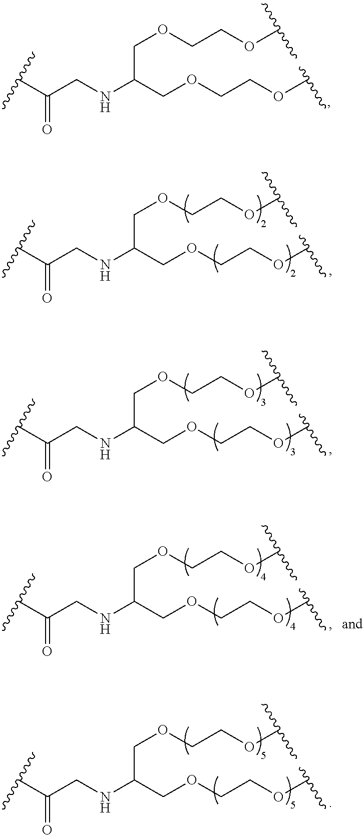

In certain embodiments Linker^{C} is selected from:

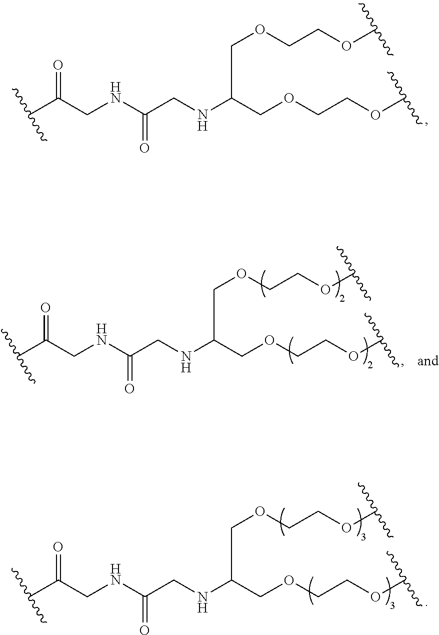

In certain embodiments Linker^{C} is selected from:

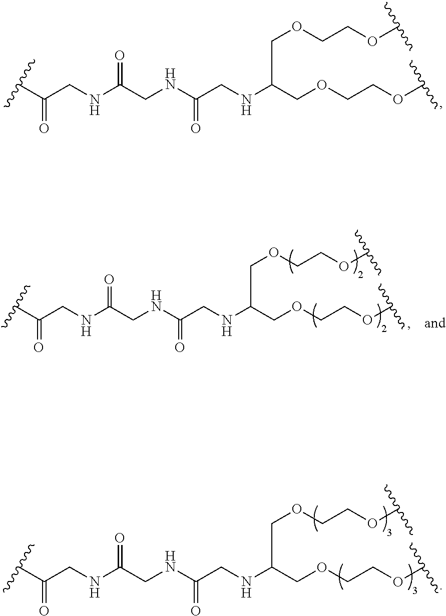

In certain embodiments Linker^{C} is selected from:

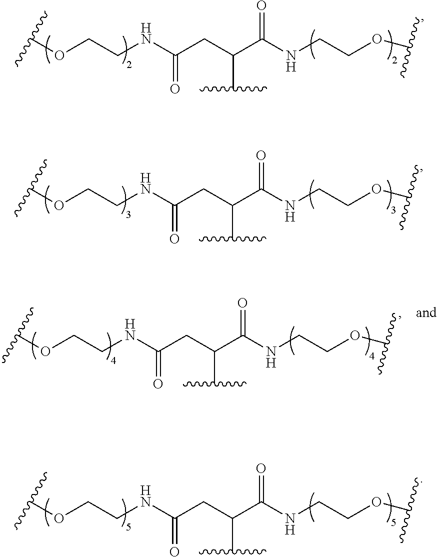

In certain embodiments Linker^{C} is selected from:

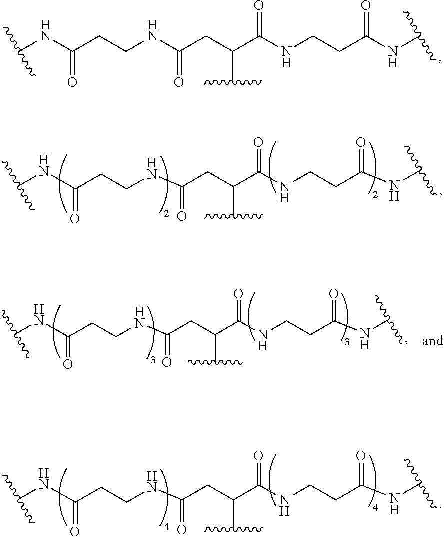

In certain embodiments Linker^{C} is selected from:

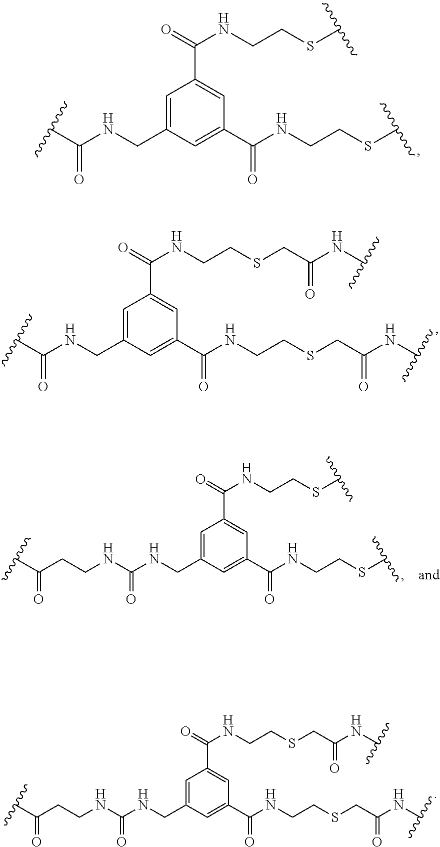 [SMILES: *SCCNC(=O)c1cc(CNC(*)=O)cc(C(=O)NCCS*)c1 ; *NC(=O)CSCCNC(=O)c1cc(CNC(*)=O)cc(C(=O)NCCSCC(=O)N*)c1 ; *SCCNC(=O)c1cc(CNC(=O)NCCC(*)=O)cc(C(=O)NCCS*)c1 ; *NC(=O)CSCCNC(=O)c1cc(CNC(=O)NCCC(*)=O)cc(C(=O)NCCSCC(=O)N*)c1]

In certain embodiments Linker^{C}-(Linker^{A})₂ is selected from:

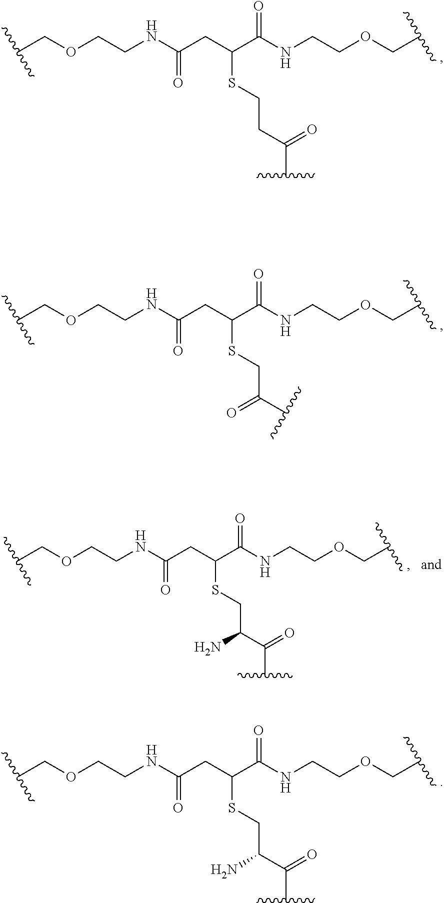 [SMILES: *COCCNC(=O)CC(SCCC(*)=O)C(=O)NCCOC* ; *COCCNC(=O)CC(SCC(*)=O)C(=O)NCCOC* ; *COCCNC(=O)CC(SC[C@H](N)C(*)=O)C(=O)NCCOC* ; *COCCNC(=O)CC(SC[C@@H](N)C(*)=O)C(=O)NCCOC*]

In certain embodiments Linker^{C}-(Linker^{A})₂ is selected from:

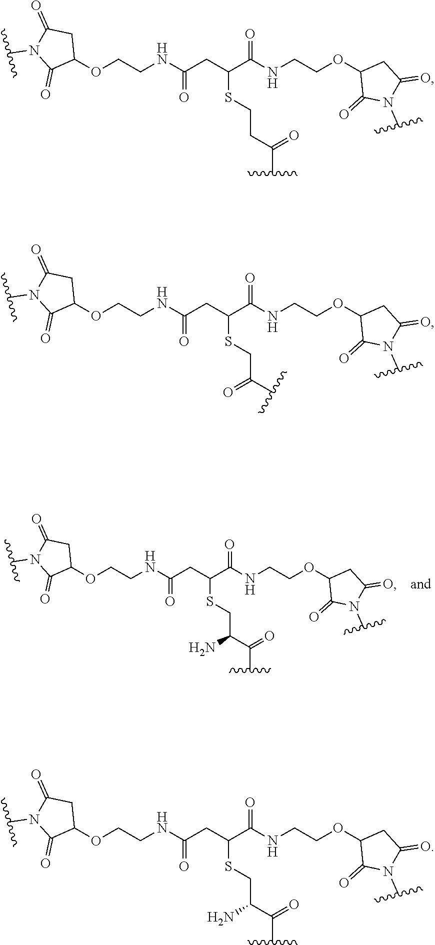 [SMILES: *C(=O)CCSC(CC(=O)NCCOC1CC(=O)N(*)C1=O)C(=O)NCCOC1CC(=O)N(*)C1=O ; *C(=O)CSC(CC(=O)NCCOC1CC(=O)N(*)C1=O)C(=O)NCCOC1CC(=O)N(*)C1=O ; *C(=O)[C@@H](N)CSC(CC(=O)NCCOC1CC(=O)N(*)C1=O)C(=O)NCCOC1CC(=O)N(*)C1=O ; *C(=O)[C@H](N)CSC(CC(=O)NCCOC1CC(=O)N(*)C1=O)C(=O)NCCOC1CC(=O)N(*)C1=O]

In certain embodiments Linker^{C}-(Linker^{A})₂ is selected from:

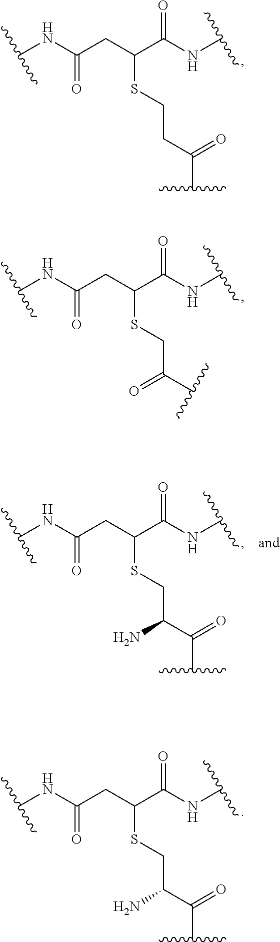 [SMILES: *NC(=O)CC(SCCC(*)=O)C(=O)N* ; *NC(=O)CC(SCC(*)=O)C(=O)N* ; *NC(=O)CC(SC[C@H](N)C(*)=O)C(=O)N* ; *NC(=O)CC(SC[C@@H](N)C(*)=O)C(=O)N*]

In certain embodiments Linker^{C}-(Linker^{A})₂ is selected from:

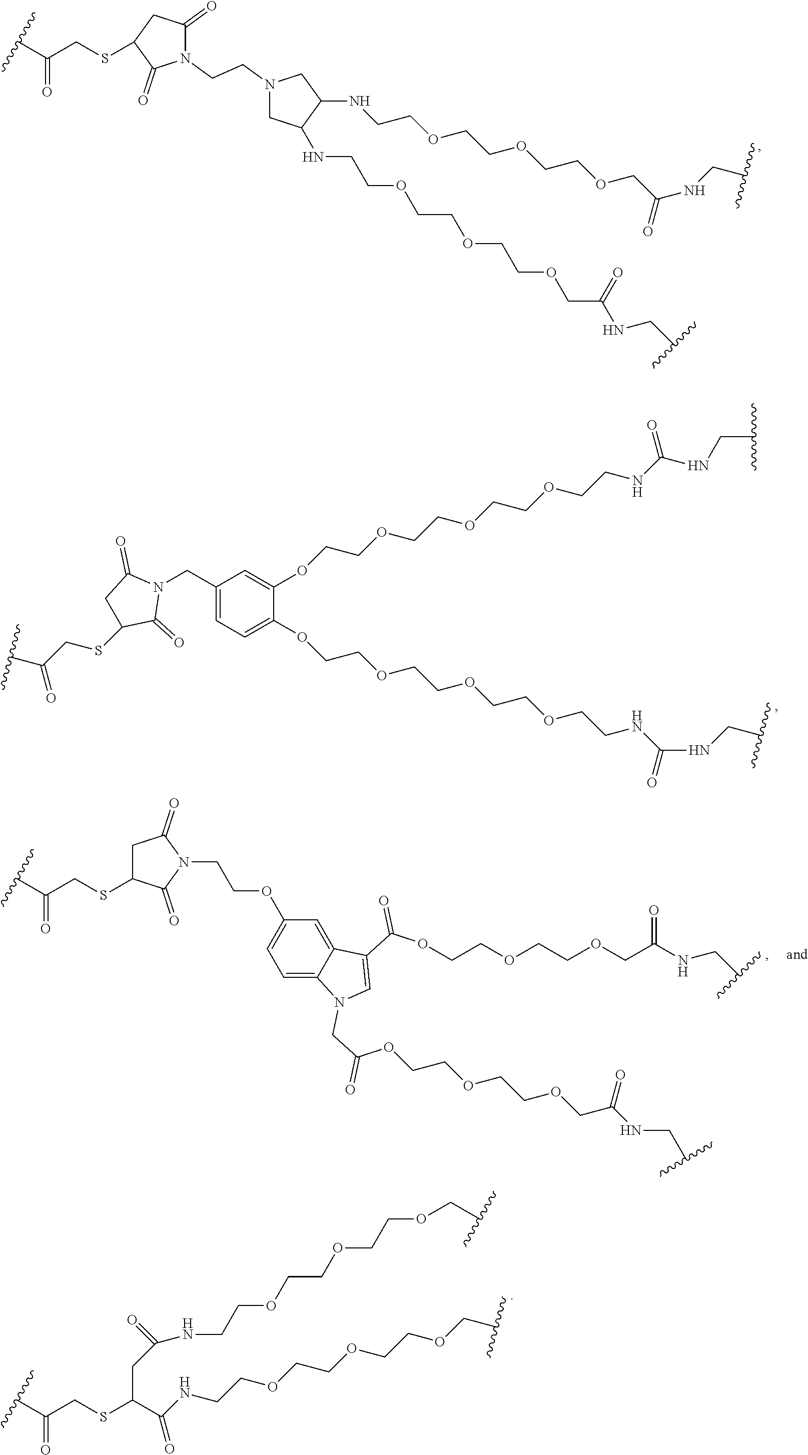 [SMILES: *CNC(=O)COCCOCCOCCNC1CN(CCN2C(=O)CC(SCC(*)=O)C2=O)CC1NCCOCCOCCOCC(=O)NC* ; *CNC(=O)NCCOCCOCCOCCOc1ccc(CN2C(=O)CC(SCC(*)=O)C2=O)cc1OCCOCCOCCOCCNC(=O)NC* ; *CNC(=O)COCCOCCOC(=O)Cn1cc(C(=O)OCCOCCOCC(=O)NC*)c2cc(OCCN3C(=O)CC(SCC(*)=O)C3=O)ccc21 ; *COCCOCCOCCNC(=O)CC(SCC(*)=O)C(=O)NCCOCCOCCOC*]

In certain embodiments, the Linker^{D} is selected from

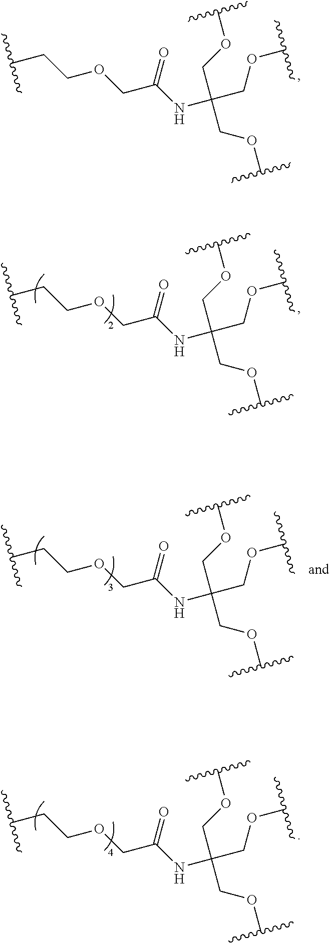

In certain embodiments, the Linker^{D} is selected from

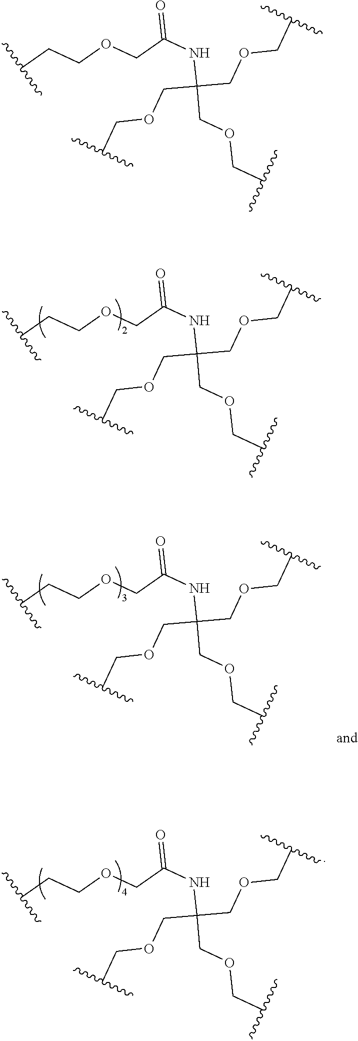

In certain embodiments, the Linker^{D} is selected from

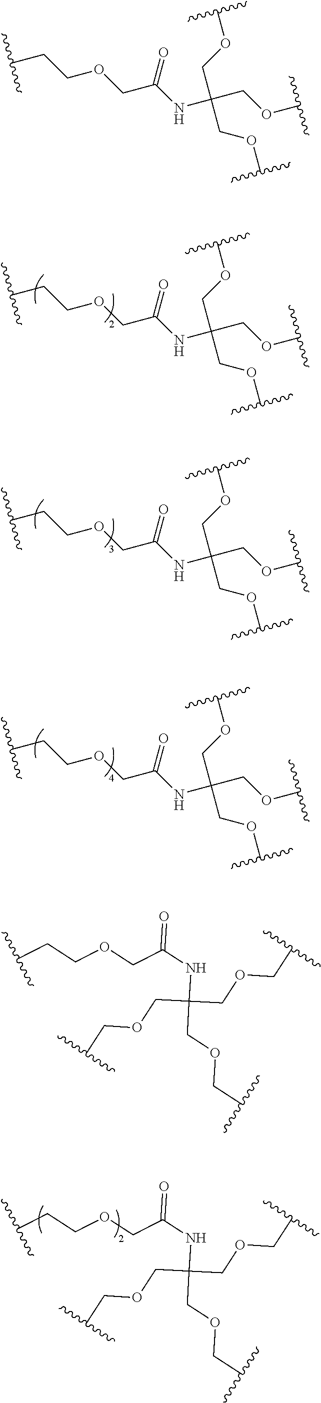

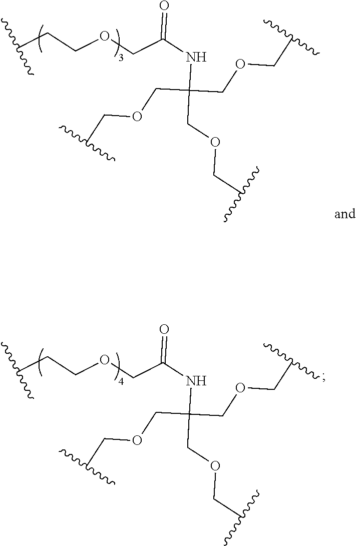

wherein each is optionally substituted with 1, 2, 3, or 4 substituents are selected from R²¹.

In certain embodiments, Linker^{B}-(Linker^{A}) is selected from

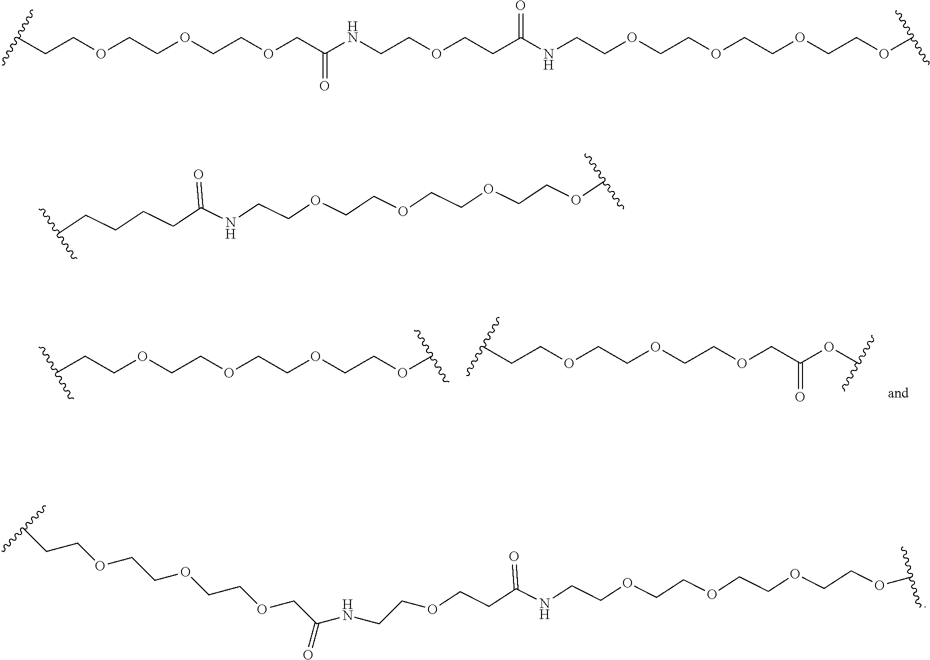 [SMILES: *CCOCCOCCOCC(=O)NCCOCCC(=O)NCCOCCOCCOCCO* ; *CCCCC(=O)NCCOCCOCCOCCO* ; *CCOCCOCCOCCO* ; *CCOCCOCCOCC(=O)O* ; *CCOCCOCCOCC(=O)NCCOCCC(=O)NCCOCCOCCOCCO*]

In certain embodiments, Linker^{C}-(Linker^{A}) is selected from

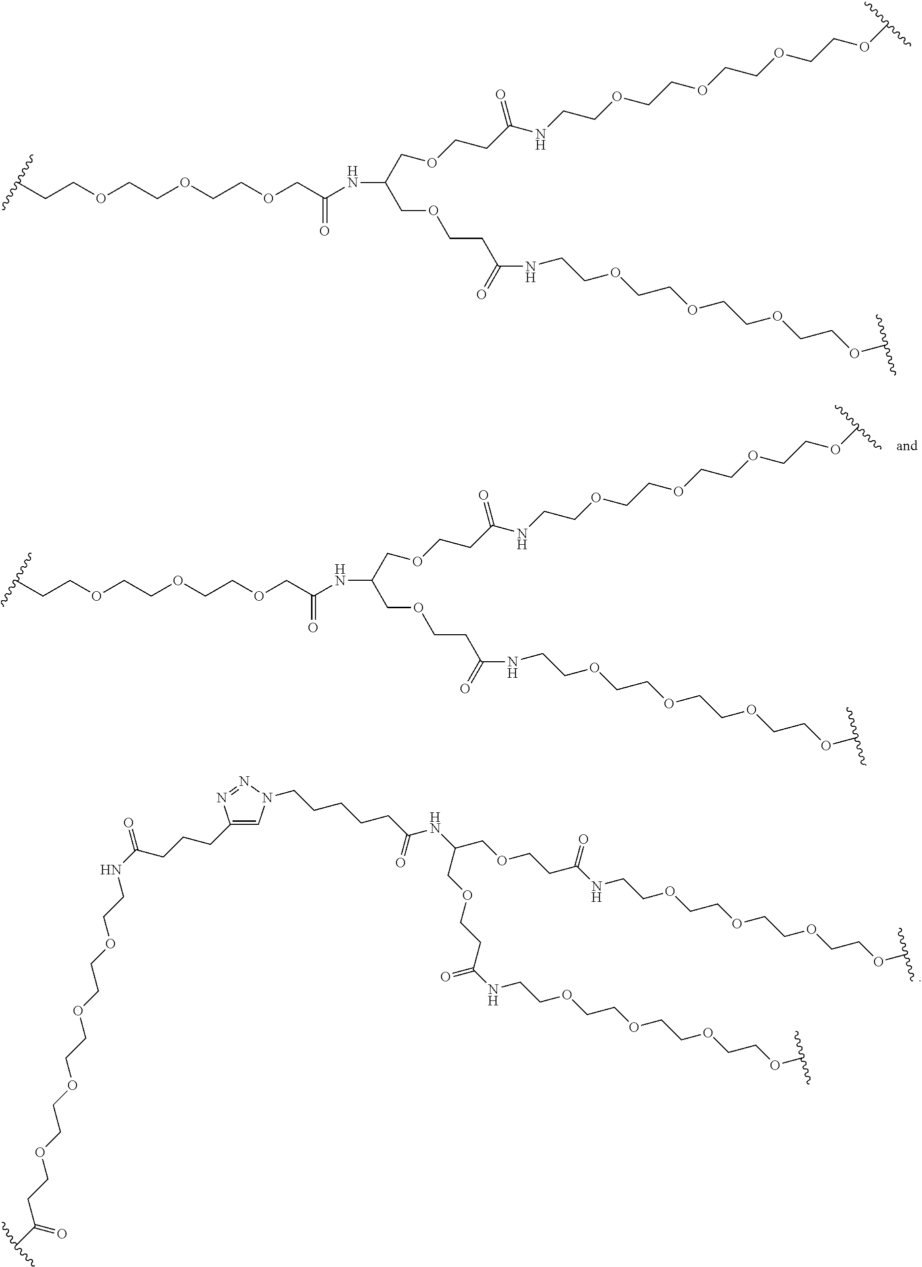 [SMILES: *CCOCCOCCOCC(=O)NC(COCCC(=O)NCCOCCOCCOCCO*)COCCC(=O)NCCOCCOCCOCCO* ; *CCOCCOCCOCC(=O)NC(COCCC(=O)NCCOCCOCCOCCO*)COCCC(=O)NCCOCCOCCOCCO* ; *OCCOCCOCCOCCNC(=O)CCOCC(COCCC(=O)NCCOCCOCCOCCO*)NC(=O)CCCCCn1cc(CCCC(=O)NCCOCCOCCOCCOCCC(*)=O)nn1]

In certain embodiments, Linker^{D}-(Linker^{A}) is selected from

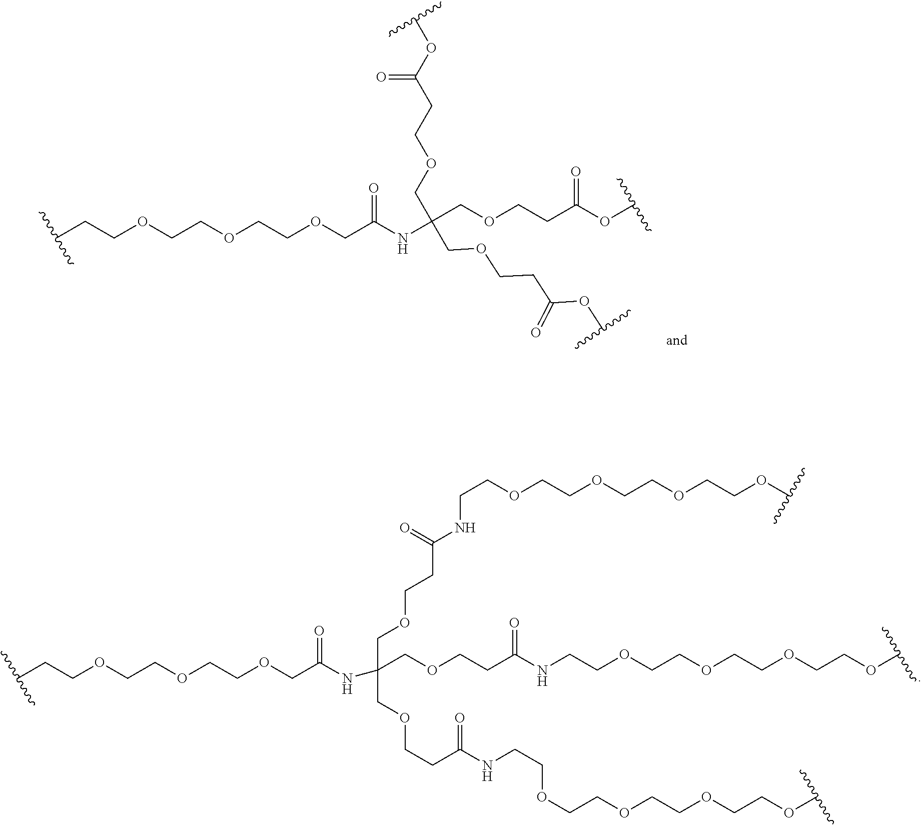 [SMILES: *CCOCCOCCOCC(=O)NC(COCCC(=O)O*)(COCCC(=O)O*)COCCC(=O)O* ; *CCOCCOCCOCC(=O)NC(COCCC(=O)NCCOCCOCCOCCO*)(COCCC(=O)NCCOCCOCCOCCO*)COCCC(=O)NCCOCCOCCOCCO*]

In certain aspects of the present invention a compound of Formula L is provided, wherein Formula L is selected from:

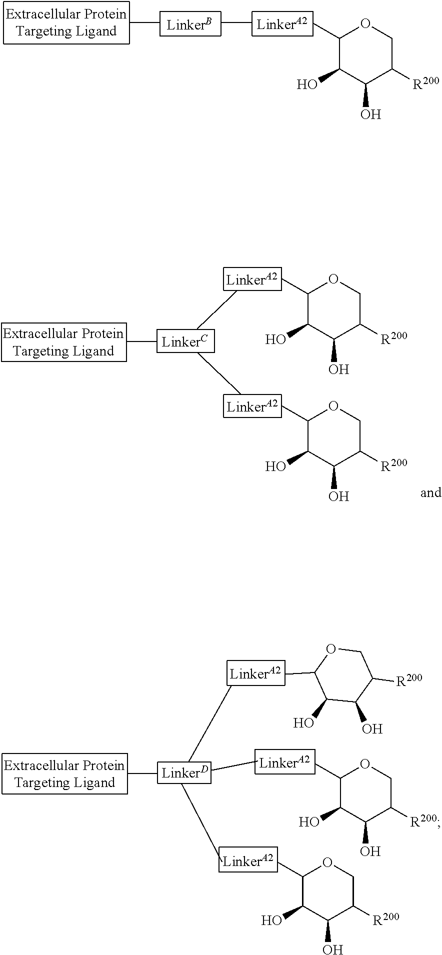

or a pharmaceutically acceptable salt thereof
wherein Linker^{A2} is selected from:

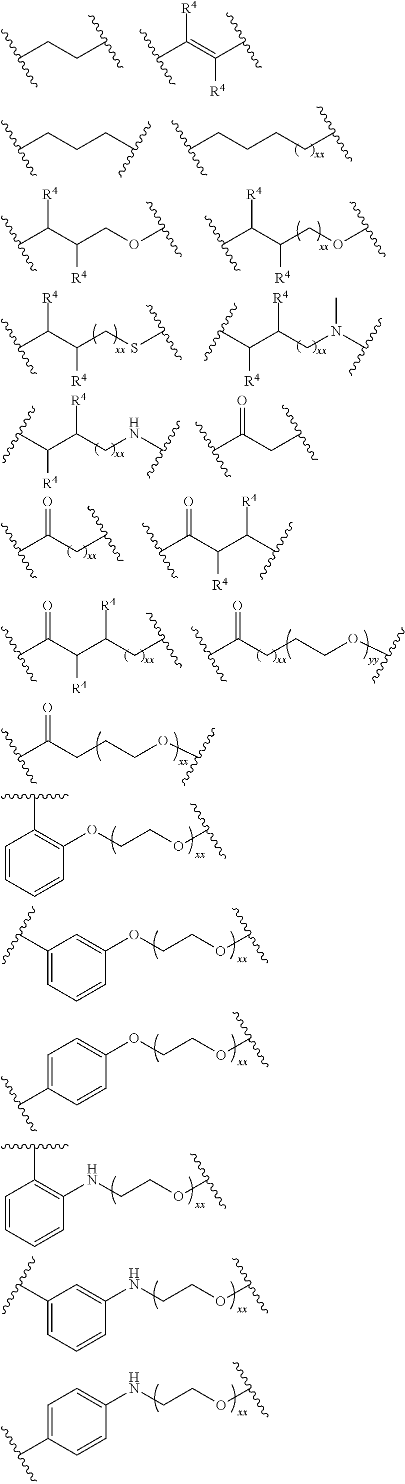

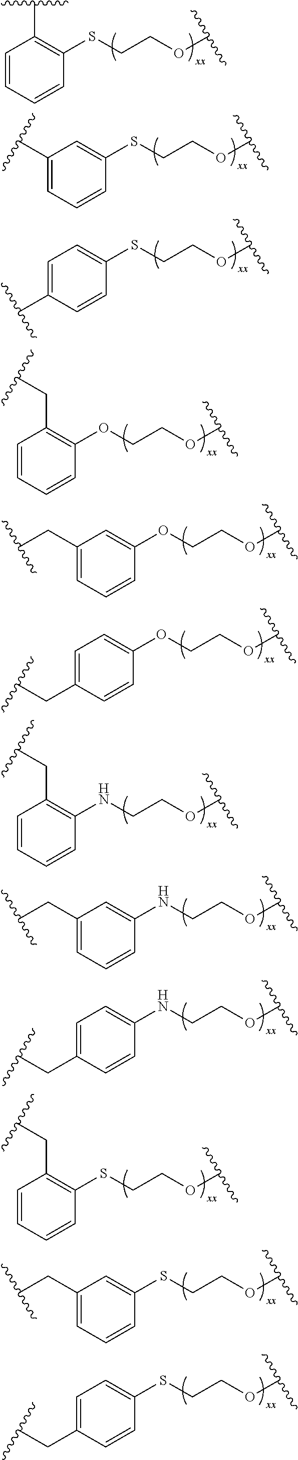

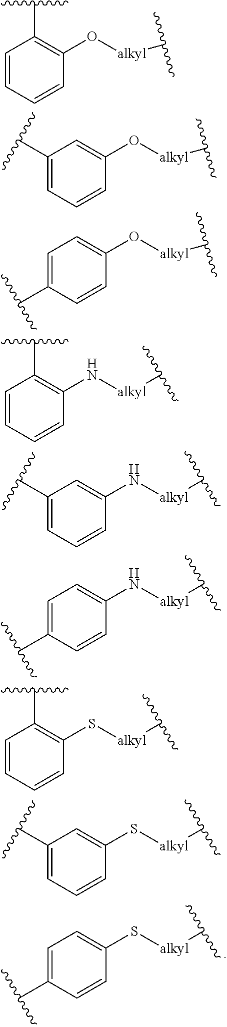

Non-limiting examples of compounds of Formula L include:

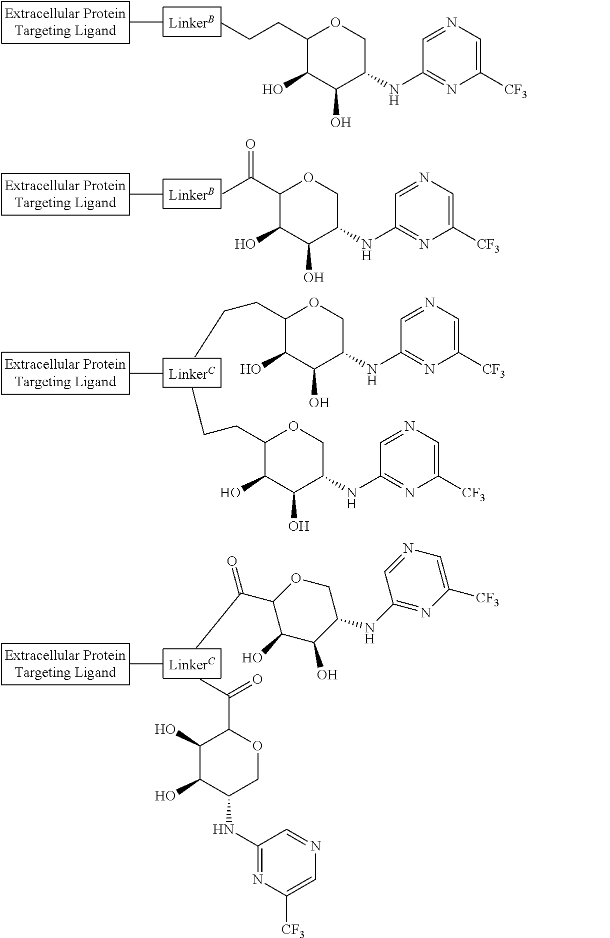

or a pharmaceutically acceptable salt thereof.

Non-limiting examples of compounds of Formula L include.

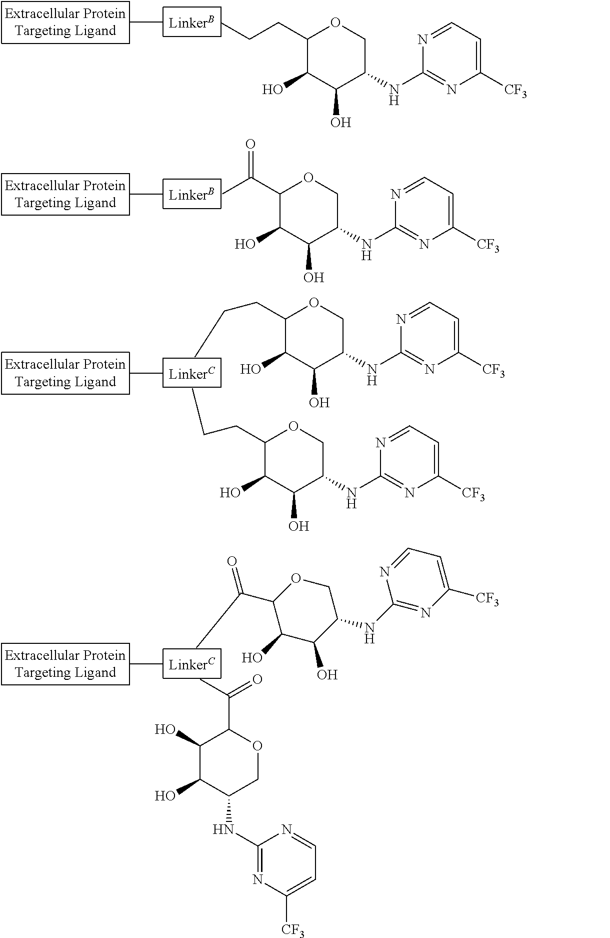

or a pharmaceutically acceptable salt thereof.

### IV. Compound Terminology

Compounds are described using standard nomenclature. Unless defined otherwise, all technical and scientific terms used herein have the same meaning as is commonly understood by one of skill in the art to which this invention belongs.

The compounds in any of the Formulas described herein include as separate embodiments enantiomers, diastereomers, tautomers, racemates, rotamers or mixtures thereof, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

The present invention includes compounds with at least one desired isotopic substitution of an atom, at an amount above the natural abundance of the isotope, i.e., enriched.

Examples of isotopes that can be incorporated into compounds of the invention include isotopes of hydrogen, carbon, nitrogen, oxygen, phosphorous, fluorine, and chlorine, such as 2H, ³H, ¹¹C, ¹³C, ¹⁴C, ¹⁵N, ¹⁷O, ¹⁸O, ¹⁸F ³¹P, ³²P, ³⁵S, ³⁶Cl, and ¹²⁵I respectively. In one embodiment, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. For example, a ¹⁸F labeled compound may be desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (2H) and tritium (3H) may optionally be used anywhere in described structures that achieves the desired result. Alternatively, or in addition, isotopes of carbon, e.g., ¹³C and ¹⁴C, may be used. In one embodiment, the isotopic substitution is replacing hydrogen with a deuterium at one or more locations on the molecule to improve the performance of the drug, for example, the pharmacodynamics, pharmacokinetics, biodistribution, half-life, stability, AUC, Tmax, Cmax, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95 or 99% or more enriched in an isotope at any location of interest. In certain embodiments deuterium is 80, 85, 90, 95 or 99% enriched at a desired location. Unless otherwise stated, the enrichment at any point is above natural abundance, and in an embodiment is enough to alter a detectable property of the drug in a human.

In one embodiment, the substitution of a hydrogen atom for a deuterium atom occurs within any variable group. For example, when any variable group is, or contain for example through substitution, methyl, ethyl, or methoxy, the alkyl residue may be deuterated (in nonlimiting embodiments, CDH₂, CD₂H, CD₃, CD₂CD₃, CHDCH₂D, CH₂CD₃, CHDCHD₂, OCDH₂, OCD₂H, or OCD₃ etc.). In certain other embodiments, a variable group has a “′” or an “a” designation, which in one embodiment can be deuterated. In certain other embodiments, when two substituents of the central core ring are combined to form a cyclopropyl ring, the unsubstituted methylene carbon may be deuterated.

The compound of the present invention may form a solvate with solvents (including water). Therefore, in one embodiment, the invention includes a solvated form of the active compound. The term “solvate” refers to a molecular complex of a compound of the present invention (including a salt thereof) with one or more solvent molecules. Nonlimiting examples of solvents are water, ethanol, dimethyl sulfoxide, acetone and other common organic solvents. The term “hydrate” refers to a molecular complex comprising a compound of the invention and water. Pharmaceutically acceptable solvates in accordance with the invention include those wherein the solvent of crystallization may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example a pyridyl group substituted by oxo is a pyridone. Combinations of substituents and/or variables are permissible only if such combinations result in stable compounds or useful synthetic intermediates.

“Alkyl” is a branched, straight chain, or cyclic saturated aliphatic hydrocarbon group. In one embodiment, the alkyl contains from 1 to about 12 carbon atoms, more generally from 1 to about 6 carbon atoms, from 1 to about 4 carbon atoms, or from 1 to 3 carbon atoms. In one embodiment, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having from 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

When a term is used that includes “alk” it should be understood that “cycloalkyl” or “carbocyclic” can be considered part of the definition, unless unambiguously excluded by the context. For example and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Nonlimiting examples are C₂-C₈alkenyl, C₂-C₇alkenyl, C₂-C₆alkenyl, C₂-C₈alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl.

“Alkynyl” is a branched or straight chain aliphatic hydrocarbon group having one or more carbon-carbon triple bonds that may occur at any stable point along the chain, for example, C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a“thioalkyl” group is an alkyl group as defined above with the indicated number of carbon atoms covalently bound through a sulfur bridge (—S—). In one embodiment, the alkoxy group is optionally substituted as described above.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. The term “aryl” includes groups where a saturated or partially unsaturated carbocycle group is fused with an aromatic ring. The term “aryl” also includes groups where a saturated or partially unsaturated heterocycle group is fused with an aromatic ring so long as the attachment point is the aromatic ring. Such compounds may include aryl rings fused to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O— or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl.

Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused or bridged with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

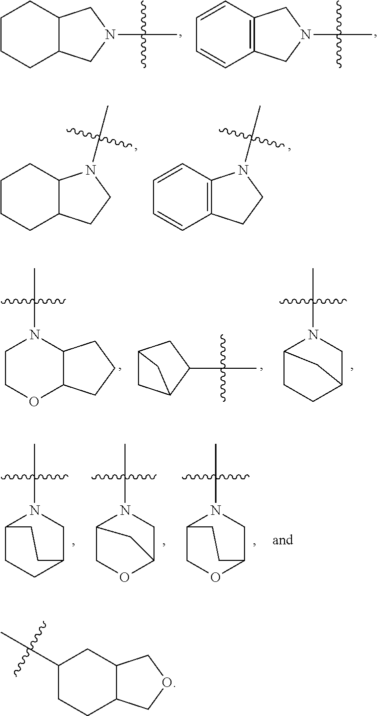

Unless otherwise drawn or clear from the context, the term “bicyclic heterocycle” includes cis and trans diastereomers. Non-limiting examples of chiral bicyclic heterocycles include:

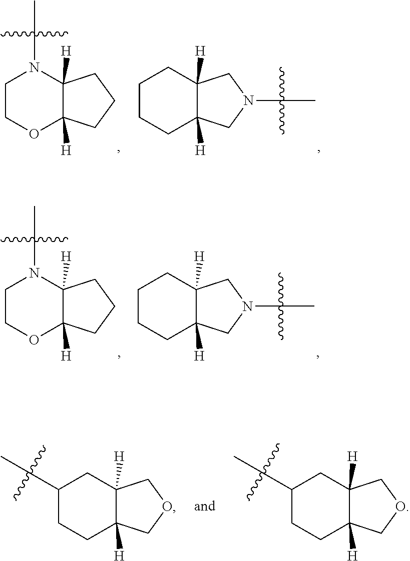

In certain alternative embodiments the term “heterocycle” refers to saturated and partially saturated heteroatom-containing ring radicals, where the heteroatoms may be selected from N, S, O, B, Si, and P.

“Heteroaryl” refers to a stable monocyclic, bicyclic, or multicyclic aromatic ring which contains from 1 to 5, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 5, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5 or 6 ring atoms. In some embodiments bicyclic heteroaryl groups are 8- to 10-membered heteroaryl groups, that is, groups containing 8 or 10 ring atoms in which one 5, 6, or 7-member aromatic ring is fused to a second aromatic or non-aromatic ring wherein the point of attachment is the aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein. “Heteroaryloxy” is a heteroaryl group as described bound to the group it substituted via an oxygen, —O—, linker.

“Heteroarylalkyl” is an alkyl group as described herein substituted with a heteroaryl group as described herein.

“Arylalkyl” is an alkyl group as described herein substituted with an aryl group as described herein.

“Heterocycloalkyl” is an alkyl group as described herein substituted with a heterocyclo group as described herein.

The term “heteroalkyl” refers to an alkyl, alkenyl, alkynyl, or haloalkyl moiety as defined herein wherein a CH₂ group is either replaced by a heteroatom or a carbon atom is substituted with a heteroatom for example, an amine, carbonyl, carboxy, oxo, thio, phosphate, phosphonate, nitrogen, phosphorus, silicon, or boron. In one embodiment, the only heteroatom is nitrogen. In one embodiment, the only heteroatom is oxygen. In one embodiment, the only heteroatom is sulfur.

In one embodiment, “heteroalkyl” is used to indicate a heteroaliphatic group (cyclic, acyclic, substituted, unsubstituted, branched or unbranched) having 1-20 carbon atoms. Nonlimiting examples of heteroalkyl moieties include polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

When a compound moiety is “optionally substituted” it may be substituted as allowed by valence with one or more groups selected from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, heteroalkyl, cyano, nitro, C(O)R³,

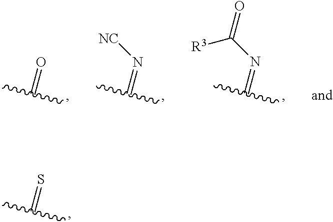

wherein the optional substituent is selected such that a stable compound results. For example

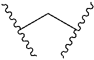 [SMILES: *C*]

could be substituted with 1 or 2 groups independently selected from alkyl, alkenyl, alkynyl, haloalkyl, —OR⁶, F, Cl, Br, I, —NR⁶R⁷, heteroalkyl, cyano, nitro, C(O)R³ so long as a stable compound results but only one group selected from

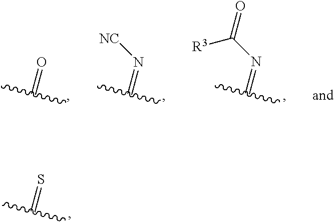

so long as a stable compound results

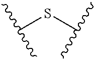 [SMILES: *S*]

on the other hand could only be substituted with 1 or 2 groups selected from

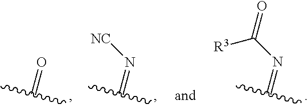

Non-limiting examples of optionally substituted CH₂ groups include:

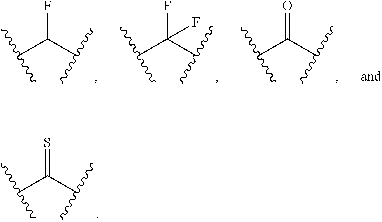

Non-limiting examples of optionally substituted —S— groups include:

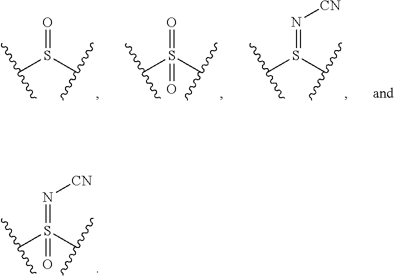

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, subcutaneous, intramuscular, parenteral, systemic, intravenous, and the like. A “dosage form” can also include an implant for controlled delivery.

“Pharmaceutical compositions” are compositions comprising at least one active agent, and at least one other substance, such as a carrier. The present invention includes pharmaceutical compositions of the described compounds.

“Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed compound in which the parent compound is modified by making an inorganic or organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic acids. Examples, of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active compound is provided.

A “pharmaceutically acceptable excipient” means an excipient that is useful in preparing a pharmaceutical composition/combination that is generally safe, acceptable for human consumption, and neither biologically nor otherwise inappropriate for administration to a host, typically a human. In one embodiment, an excipient is used that is acceptable for veterinary use.

A “patient” or “host” or “subject” is a human or non-human animal in need of treatment or prevention of any of the disorders as specifically described herein. Typically, the host is a human. A “patient” or “host” or “subject” also refers to for example, a mammal, primate (e.g., human), cow, sheep, goat, horse, dog, cat, rabbit, rat, mice, bird and the like.

A “therapeutically effective amount” of a compound, pharmaceutical composition, or combination of this invention means an amount effective, when administered to a host, that provides a therapeutic benefit such as an amelioration of symptoms or reduction or diminution of the disease itself. In another aspect, a preventative amount can be administered that prevents or minimizes the risk of the disease mediated by the Extracellular Target Protein.

### Embodiments of “Alkyl”

In one embodiment “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In one embodiment “alkyl” has one carbon.

In one embodiment “alkyl” has two carbons.

In one embodiment “alkyl” has three carbons.

In one embodiment “alkyl” has four carbons.

In one embodiment “alkyl” has five carbons.

In one embodiment “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment the “alkyl” group is optionally substituted.

In an alternative embodiment the “alkenyl” group is optionally substituted.

In an alternative embodiment the “alkynyl” group is optionally substituted.

### Embodiments of “Haloalkyl”

In one embodiment “haloalkyl” is a C₁-C₁₀haloalkyl, C₁-C₉haloalkyl, C₁-C₈haloalkyl, C₁-C₇haloalkyl, C₁-C₆haloalkyl, C₁-C₈haloalkyl, C₁-C₄haloalkyl, C₁-C₃haloalkyl, and C₁-C₂haloalkyl.

In one embodiment “haloalkyl” has one carbon.

In one embodiment “haloalkyl” has one carbon and one halogen.

In one embodiment “haloalkyl” has one carbon and two halogens.

In one embodiment “haloalkyl” has one carbon and three halogens.

In one embodiment “haloalkyl” has two carbons.

In one embodiment “haloalkyl” has three carbons.

In one embodiment “haloalkyl” has four carbons.

In one embodiment “haloalkyl” has five carbons.

In one embodiment “haloalkyl” has six carbons.

Non-limiting examples of “haloalkyl” include:

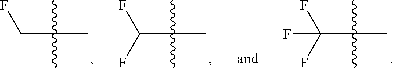

Additional non-limiting examples of “haloalkyl” include:

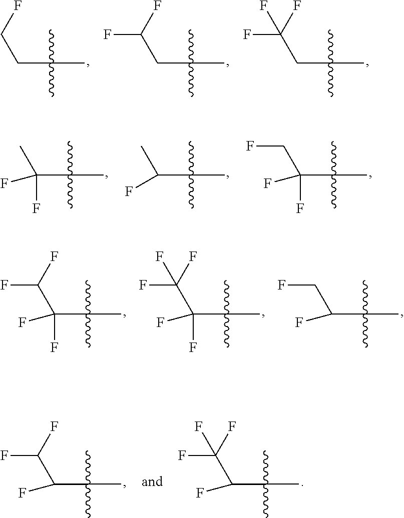

Additional non-limiting examples of “haloalkyl” include:

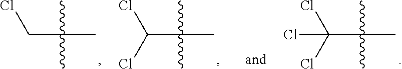

Additional non-limiting examples of “haloalkyl” include:

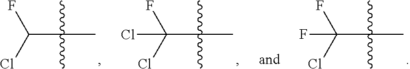

### Embodiments of “Heteroaryl”

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

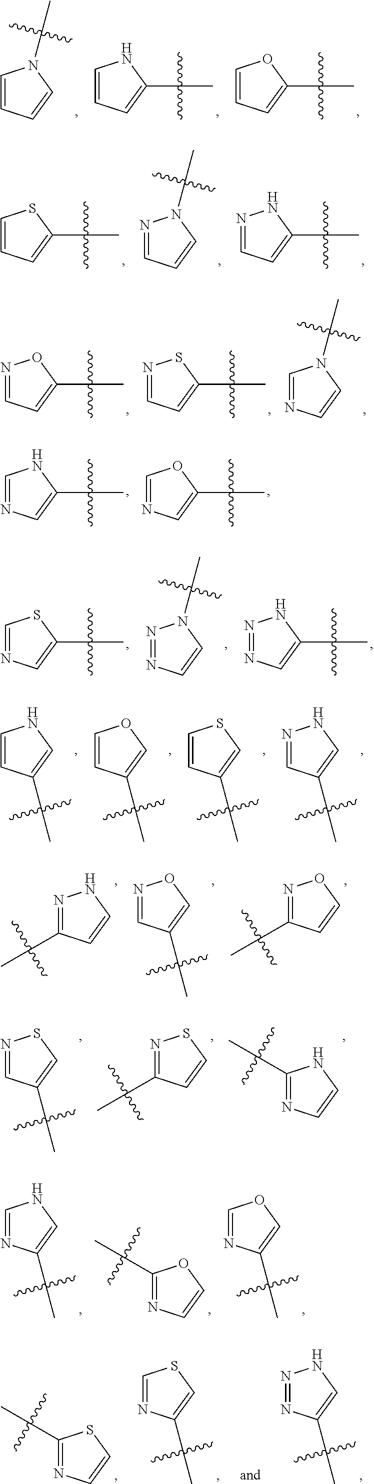

In one embodiment “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

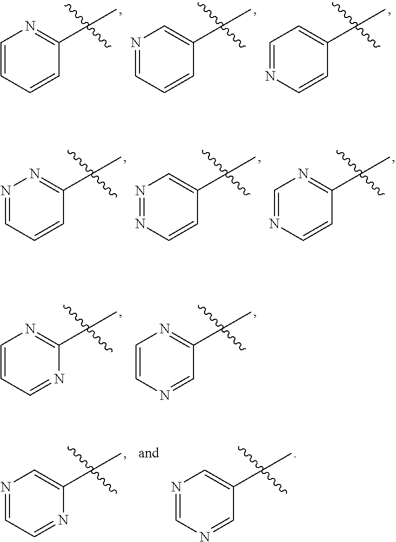

Additional non-limiting examples of heteroaryl groups include:

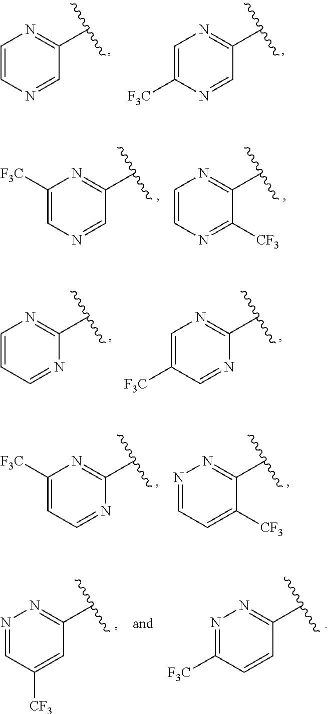

In one embodiment “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

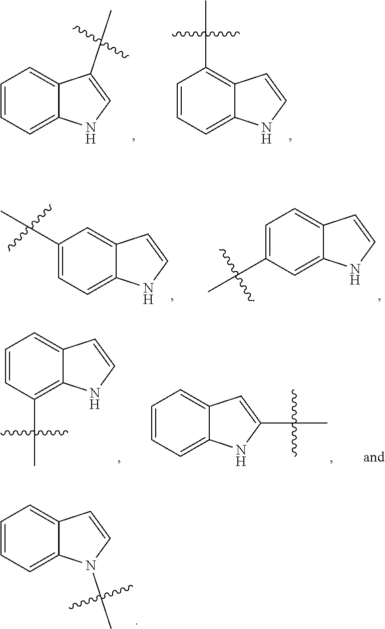

Additional non-limiting examples of “heteroaryl” groups that are bicyclic include:

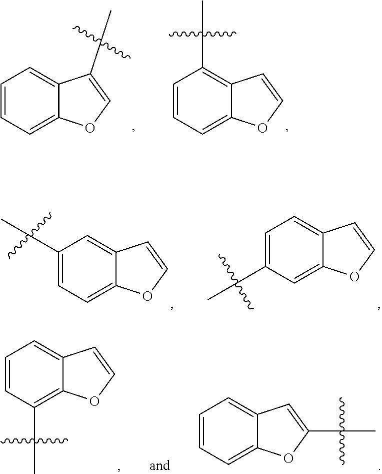

Additional non-limiting examples of “heteroaryl” groups that are bicyclic include:

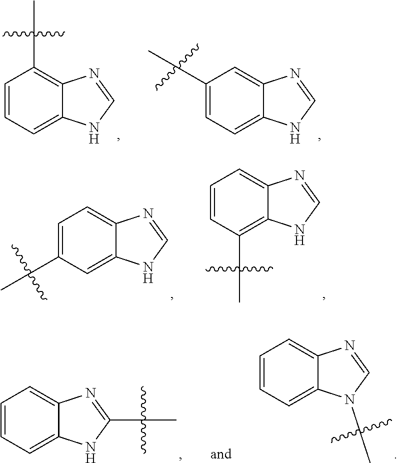

In one embodiment “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

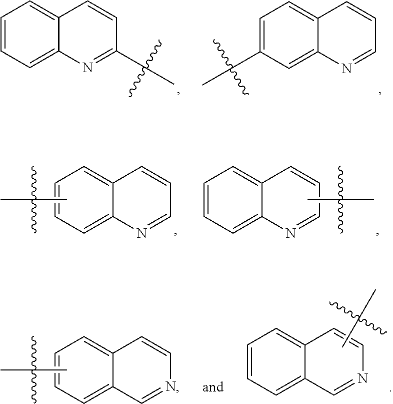

In certain embodiments “heteroaryl” refers to a stable monocyclic, bicyclic, or multicyclic aromatic ring which contains from 1 to 3, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon.

### Embodiments of “Heterocycle”

In one embodiment “heterocycle” refers to a cyclic ring with one nitrogen and 3, 4, 5, 6, 7, or 8 carbon atoms.

In one embodiment “heterocycle” refers to a cyclic ring with one nitrogen and one oxygen and 3, 4, 5, 6, 7, or 8 carbon atoms.

In one embodiment “heterocycle” refers to a cyclic ring with two nitrogens and 3, 4, 5, 6, 7, or 8 carbon atoms.

In one embodiment “heterocycle” refers to a cyclic ring with one oxygen and 3, 4, 5, 6, 7, or 8 carbon atoms.

In one embodiment “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

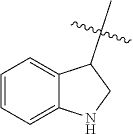 [SMILES: *C1CNc2ccccc21]

is a “heterocycle” group.

However,

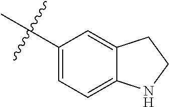 [SMILES: *c1ccc2c(c1)CCN2]

is an “aryl” group.

Non-limiting examples of “heterocycle” also include:

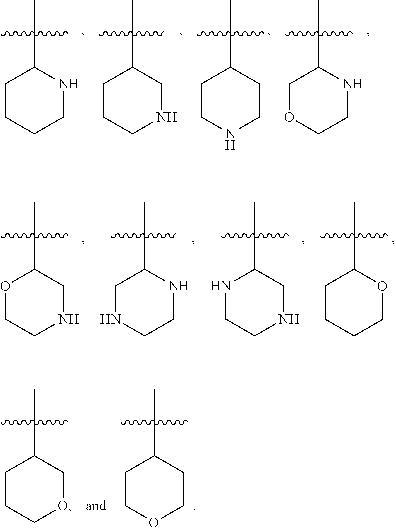

Additional non-limiting examples of “heterocycle” include:

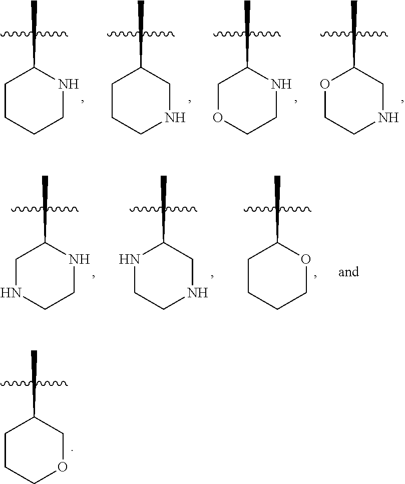

Additional non-limiting examples of “heterocycle” include

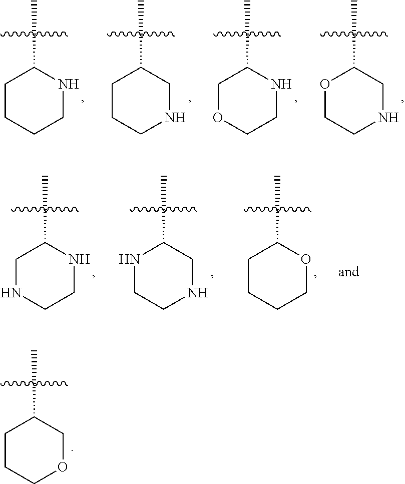

Non-limiting examples of “heterocycle” also include:

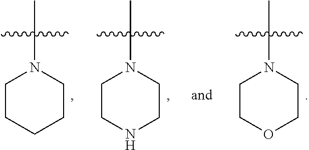

Non-limiting examples of “heterocycle” also include:

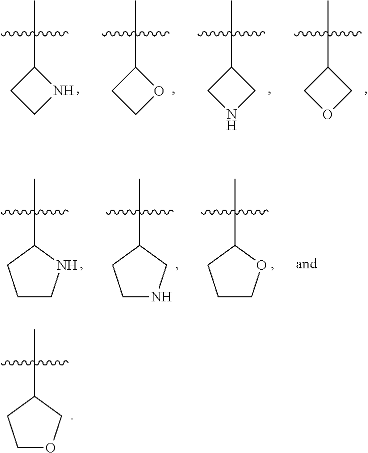

Additional non-limiting examples of “heterocycle” include:

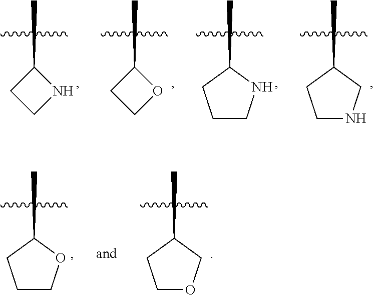

Additional non-limiting examples of “heterocycle” include:

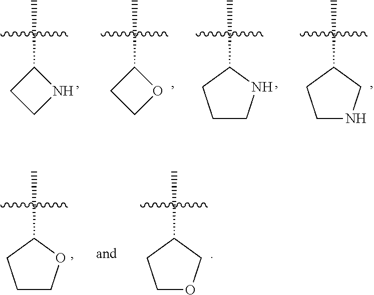

### Aryl

In one embodiment “aryl” is a 6 carbon aromatic group (phenyl).

In one embodiment “aryl” is a 10 carbon aromatic group (naphthyl).

In one embodiment “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

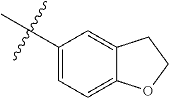 [SMILES: *c1ccc2c(c1)CCO2]

is an “aryl” group.

However,

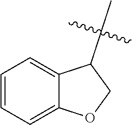 [SMILES: *C1COc2ccccc21]

is a “heterocycle” group.

### Embodiments of “Arylalkyl”

Non-limiting examples of “arylalkyl” include:

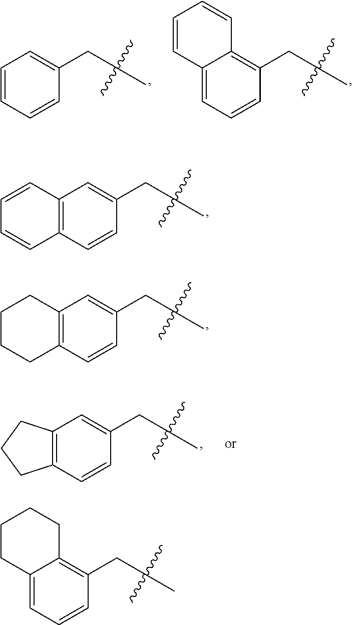

In one embodiment “arylalkyl” is

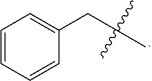 [SMILES: *Cc1ccccc1]

In one embodiment the “arylalkyl” refers to a 2 carbon alkyl group substituted with an aryl group.

Non-limiting examples of “arylalkyl” include:

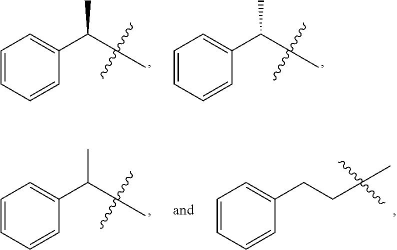

### V. Extracellular Protein Degradation

A wide range of well-known and characterized extracellular proteins can cause, modulate, or amplify diseases in vivo, such as abnormal cellular proliferation such as tumors and cancer, autoimmune disorders, inflammation and aging-related diseases. For example, extracellular proteins such as growth factors, cytokines, and chemokines bind to cell surface receptors, often initiate aberrant signaling in multiple diseases such as cancer and inflammation.

An extracellular protein degrader described herein or its pharmaceutically acceptable salt and/or its pharmaceutically acceptable compositions can be used to treat a disorder which is mediated by the Target Extracellular Protein that binds to the Extracellular Protein Targeting Ligand. The described degraders are capable of targeting specific Extracellular Proteins that mediate pathological disorders for lysosomal degradation. The Target Extracellular Protein may modulate a disorder in a human via a mechanism of action such as modification of a biological pathway, pathogenic signaling, or modulation of a signal cascade or cellular entry. In one embodiment, the Target Extracellular Protein is a protein that is not druggable in the classic sense in that it does not have a binding pocket or an active site that can be inhibited or otherwise bound, and cannot be easily allosterically controlled. In another embodiment, the Target Extracellular Protein is a protein that is druggable in the classic sense, yet for therapeutic purposes, degradation of the protein is preferred to inhibition. The Target Extracellular Protein is recruited with an Extracellular Protein Targeting Ligand, which is a ligand for the Target Extracellular Protein.

Typically, the Extracellular Protein Targeting Ligand binds the Target Extracellular Protein in a non-covalent fashion. In an alternative embodiment, the Target Extracellular Protein is covalently bound to the Extracellular Protein Targeting Ligand in a covalent manner that can be irreversible or reversible.

Accordingly, in some embodiments, a method to treat a host with a disorder mediated by a Target Extracellular Protein is provided that includes administering an effective amount of a degrader targeting the Target Extracellular Protein to the host, typically a human, optionally in a pharmaceutically acceptable composition.

The Target Extracellular Protein can be any amino acid sequence to which the degrader comprising an Extracellular Protein Targeting Ligand can be bound which through degradation thereof, results in a beneficial therapeutic effect. In one embodiment, the Target Extracellular Protein is a non-endogenous peptide such as that from a pathogen or toxin. In another embodiment, the Target Extracellular Protein can be an endogenous protein that mediates a disorder. The endogenous protein can be either the normal form of the protein or an aberrant form. For example, the Target Extracellular Protein can be an extracellular mutant protein, or a protein, for example, where a partial, or full, gain-of-function or loss-of-function is encoded by nucleotide polymorphisms. In some embodiments, the degrader targets the aberrant form of the protein and not the normal form of the protein.

The Extracellular Protein Targeting Ligand is a ligand which covalently or non-covalently binds to a Target Extracellular Protein which has been selected for lysosomal degradation. In certain embodiments the Extracellular Protein Targeting Ligand is a small molecule or moiety (for example a peptide, nucleotide, antibody fragment, aptamer, biomolecule, or other chemical structure) that binds to a Target Extracellular Protein, and wherein the Target Extracellular Protein is a mediator of disease in a host as described in detail below. Exemplary Extracellular Protein Targeting Ligands are provided in the Figures.

### Anchor Bond

The Extracellular Protein Targeting Ligand (“EPTL”) is covalently bound to Linker in the protein degrader compound through the Anchor Bond (which is the chemical bond between the EPTL and either Linker^{B}, Linker^{C} or Linker^{D}). This bond can be placed at any location on the ligand that does not unacceptably disrupt the ability of the EPTL to bind to the Target Extracellular Protein. The Anchor Bond is depicted on the nonlimiting examples of Extracellular Protein Target Ligands in the figures as:

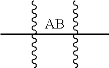 [SMILES: **]

A number of exemplary Target Extracellular Proteins for medical therapy described below have characterizing structural information in the well-known Protein Data Bank (“PDB”), which is a database for the three-dimensional structural information for large biological molecules such as proteins and nucleic acids. PDB includes x-ray crystallography and other information submitted by scientists around the world, and is freely accessible. See for example www.rcsb.org; www.wwpdb.org and www.uniprot.org. Using the PDB codes for example provided in Section ** or in the Data Bank itself, and technical references provided herein or otherwise publicly available, the skilled artisan can determine appropriate locations where the EPTL can be linked through an Anchor Bond to Linker^{B}, Linker^{C} or Linker^{D} to the receptor-binding moiety. For many of these proteins, published references describe how a range of ligands bind to the Target Extracellular Proteins, and from this information, one can determine reasonable Anchor Bond locations.

For example, the skilled artisan can use available visualization tools, including those available on the PDB website, to determine where the Extracellular Protein Targeting Ligand docks into to the Target Extracellular Protein. The skilled artisan can also import the crystal structure and the selected Extracellular Protein Targeting Ligand of interest into modeling software (including for example PyMOL, Glide, Maestro, RasMol, Visual Molecular Dynamics, Jmol, and AutoDock) to determine what portion of the Extracellular Protein Targeting Ligand is bound to the Target Extracellular Protein. The ASGPR or mannose 6-phosphate receptor ligand is then bound through the Linker and the Anchor Bond at a point that does not unduly adversely affect binding to the Target Extracellular Protein.

### Optional Substituents

In certain embodiments an Extracellular Protein Targeting Ligand described herein, for example in one of the figures or below is optionally substituted with 1, 2, 3, or 4 optional substituents independently selected from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, heteroalkyl, cyano, nitro, C(O)R³,

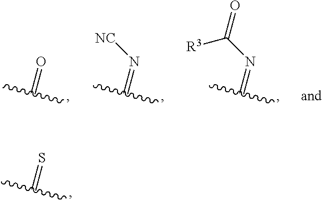

wherein the optional substituent is selected such that a stable compound results.

In certain embodiments the Target Extracellular Protein is selected from IgA, IgG, IgE, TNF-alpha, IL-1, IL-2, IL-6, IFN-γ, VEGF, TGF-β1, PCSK-9, CPB2, ChE, CCL2, Factor VII, Factor IX, CD40L, Factor Xa, Factor XI, Factor XIa, Factor XII, Factor XIII, FGF1, FGF2, FN1, IL-5, IL-8, IL-10, IL-21, IL-22, Kallikrein 1, LPL, MMP1, MIF, GIF, L-dopachrome isomerase, or phenylpyruvate tautomerase, neutrophil elastase, Prothrombin, KLKB1, PLG, PAI-1, endothelial plasminogen activator inhibitor, serpin E1, phospholipases A2, PLA2, PA21B, PLA2G1B, PLA2-IB, PLA2, PLA2A, PA2IIA, PLA2G2A, PLA2-IIA, PGF, plasminogen activator, tissue type (tPA, PLAT), Transforming growth factor beta 2 (TGF-β2, TGFB2), thrombospondin 1, Urokinase, Urokinase-type plasminogen activator, complement factor B, complement factor D, target complement factor H, and complement component 5.

In certain embodiments, where the Target Extracellular Protein has a receptor the Target Extracellular Protein can be used to degrade the receptor.

In certain embodiments the Extracellular Protein Targeting Ligand is selected from IgA, IgG, IgE, TNF-alpha, IL-1, IL-2, IL-6, IFN-γ, VEGF, TGF-β1, PCSK-9, CPB2, ChE, CCL2, Factor VII, Factor IX, CD40L, Factor Xa, Factor XI, Factor XIa, Factor XII, Factor XIII, FGF1, FGF2, FN1, IL-5, IL-8, IL-10, IL-21, IL-22, Kallikrein 1, LPL, MMP1, MIF, GIF, L-dopachrome isomerase, or phenylpyruvate tautomerase, neutrophil elastase, Prothrombin, KLKB1, PLG, PAI-1, endothelial plasminogen activator inhibitor, serpin E1, phospholipases A2, PLA2, PA21B, PLA2G1B, PLA2-IB, PLA2, PLA2A, PA2IIA, PLA2G2A, PLA2-IIA, PGF, plasminogen activator, tissue type (tPA, PLAT), Transforming growth factor beta 2 (TGF-β2, TGFB2), thrombospondin 1, Urokinase, Urokinase-type plasminogen activator, complement factor B, complement factor D, target complement factor H, and complement component 5.

### Amino Acids

In certain embodiments the Extracellular Protein Targeting Ligand comprises one or more amino acids. The invention contemplates using natural amino acids, unnatural amino acids, or any combination thereof to achieve desired targeting ligand properties.

The term “natural amino acid” refers to an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

In certain embodiments a natural amino acid is replaced with a corresponding unnatural amino acid for example substituting a phenylalanine for a 4-chloro-phenylalanine. Non-limiting examples of unnatural amino acids include: 4-chloro-phenylalanine, 3-fluoro-phenylalanine, 4-trifluoromethyl-phenylalanine, 3,4-dichloro-phenylalanine, 4-phenyl-phenylalanine, N-methylalanine, N-methylglutamic acid, N-methylphenylalanine, and homoserine.

Additional examples of non-natural amino acids include.

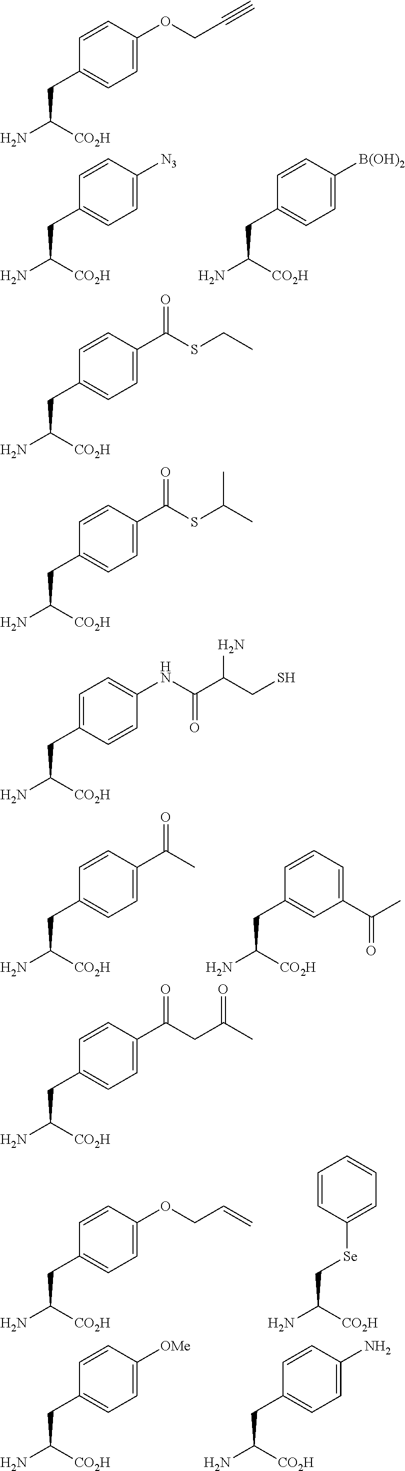

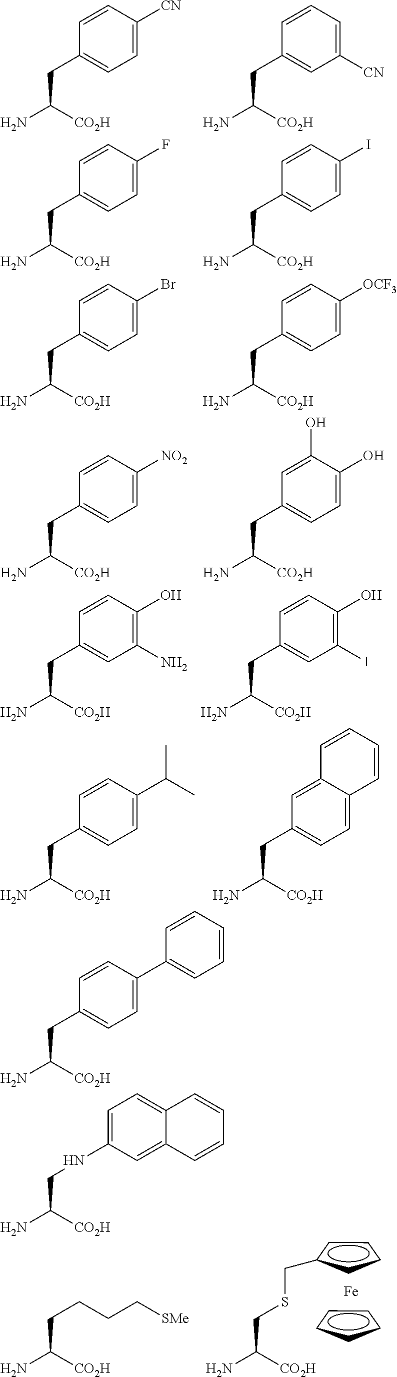

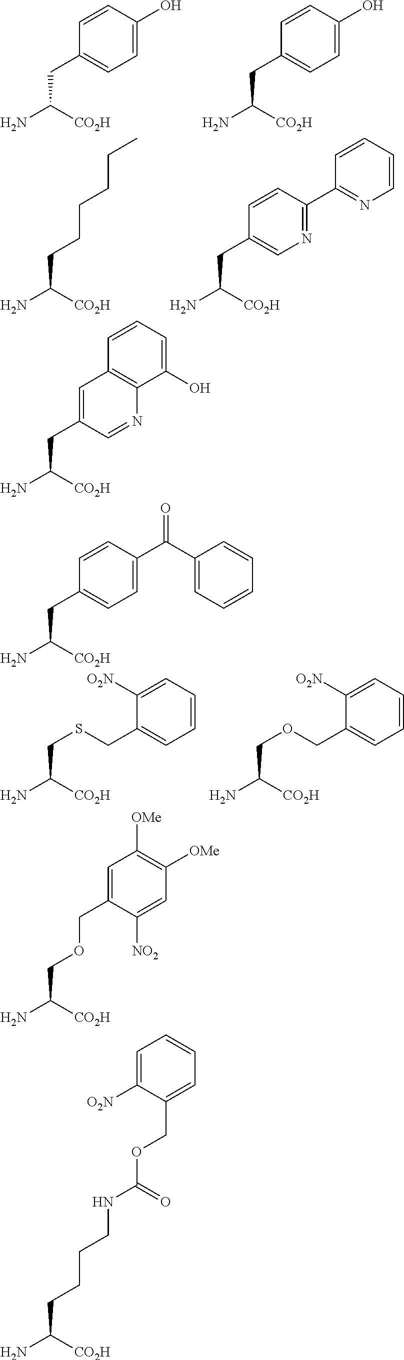

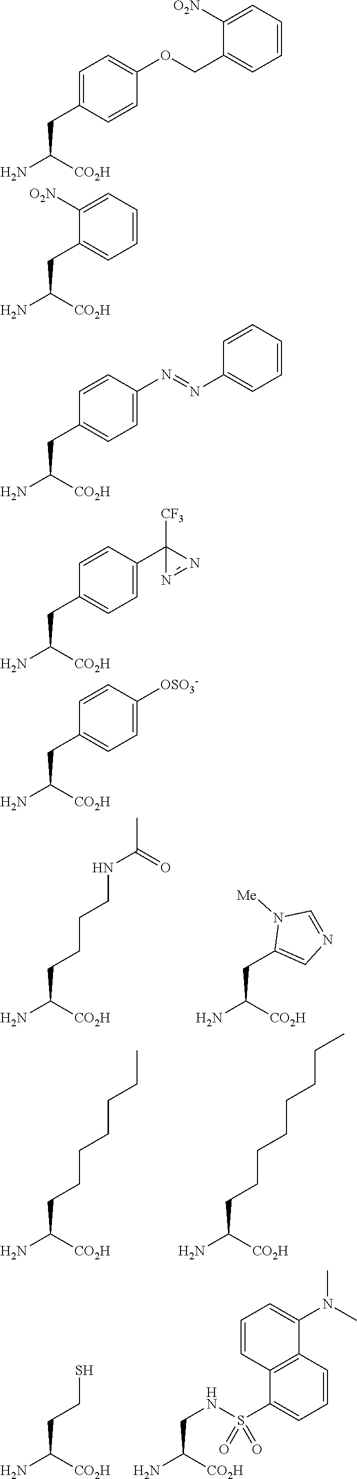

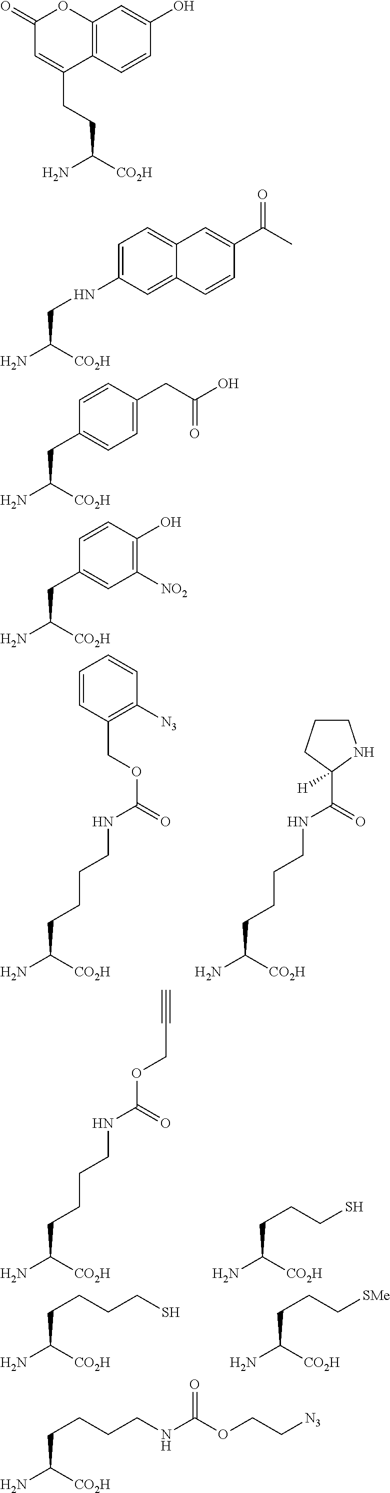

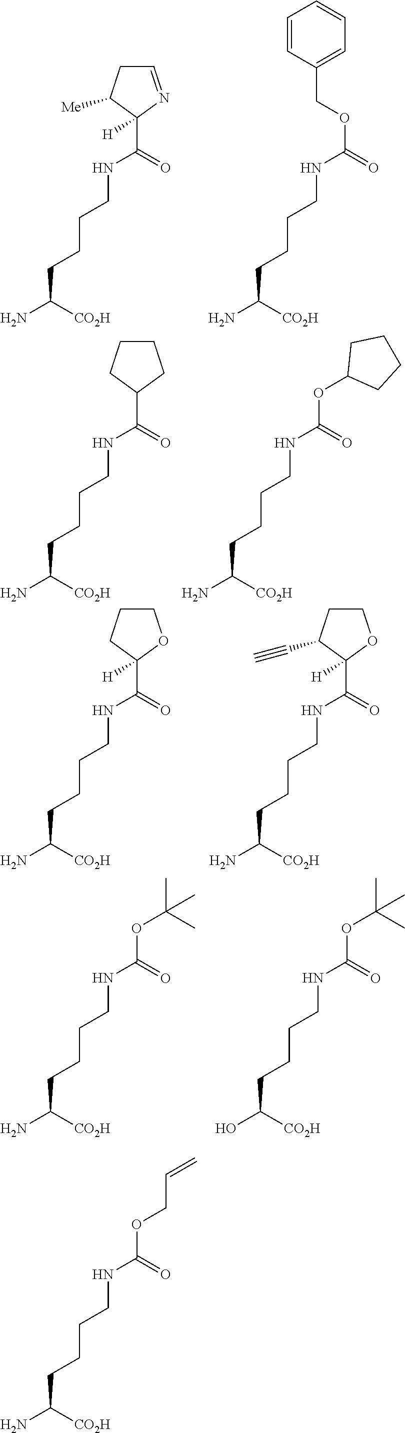

In certain embodiments the Extracellular Protein Targeting Ligand is a sequence of amino acids. In certain embodiments the amino acid sequence is connected to the Linker portion of the molecule by a bond to a terminal amine. In certain embodiments the amino acid sequence is connected to the Linker portion of the molecule by a bond to a terminal carboxylic acid (e.g. an ester or amide). In certain embodiments the peptide includes an amine, hydroxyl, or carboxylic acid side chain and the linker may be bound to one of these sidechains.

For example, when the amino acid sequence is SEQ ID NO: 1 MLKKIE non-limiting examples of locations wherein the peptide may be attached to the linker include:

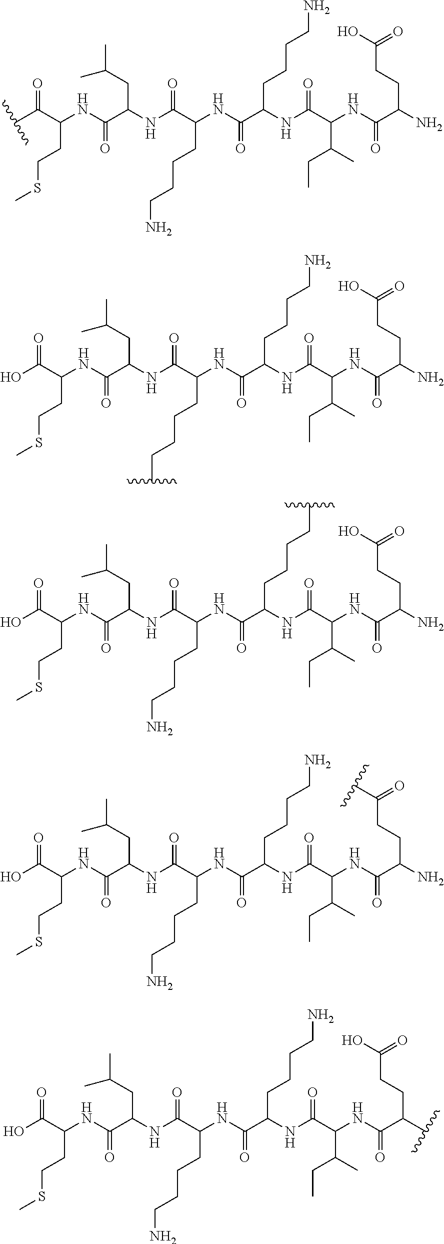 [SMILES: *C(=O)C(CCSC)NC(=O)C(CC(C)C)NC(=O)C(CCCCN)NC(=O)C(CCCCN)NC(=O)C(NC(=O)C(N)CCC(=O)O)C(C)CC ; *CCCCC(NC(=O)C(CCCCN)NC(=O)C(NC(=O)C(N)CCC(=O)O)C(C)CC)C(=O)NC(CC(C)C)C(=O)NC(CCSC)C(=O)O ; *CCCCC(NC(=O)C(NC(=O)C(N)CCC(=O)O)C(C)CC)C(=O)NC(CCCCN)C(=O)NC(CC(C)C)C(=O)NC(CCSC)C(=O)O ; *C(=O)CCC(N)C(=O)NC(C(=O)NC(CCCCN)C(=O)NC(CCCCN)C(=O)NC(CC(C)C)C(=O)NC(CCSC)C(=O)O)C(C)CC ; *C(CCC(=O)O)C(=O)NC(C(=O)NC(CCCCN)C(=O)NC(CCCCN)C(=O)NC(CC(C)C)C(=O)NC(CCSC)C(=O)O)C(C)CC]

The amino acid sequence can be attached to the Linker with chemistry described herein and as otherwise known in the art. For example, when the desired linking group is an amide the linker can be presented with an amine, carboxylic acid, ester or other amide precursor and the targeting ligand can be attached with an amide coupling reaction such as a HATU or HBTU coupling reaction.

Non-limiting examples of Extracellular Protein Targeting Ligands that are a sequence of amino acids include aptamers, antibodies, and peptides. In certain embodiments the left most amino acid listed in the sequence listing is the C-terminus. In other embodiments the right most amino acid listed in the sequence listing is the C-terminus.

In certain embodiments the amino acid sequence refers to a sequence without specified chirality. In other embodiments the amino acid sequence is all D-, all L-, or a mixture of D- and L-amino acids.

### TNF-Alpha (TNF-α)

In some embodiments, the Target Extracellular Protein is human TNF-α (UniProtKB-P01375 (TNFA_HUMAN)). TNF-α is a pro-inflammatory cytokine active in the bodily immune response and serious inflammatory diseases. TNF-α has been implicated in a number of disorders, including but not limited to rheumatoid arthritis, inflammatory bowel disease, graft-vs-host disease, ankylosing spondylitis, psoriasis, hidradenitis suppurativa, refractory asthma, systemic lupus erthyematosus, diabetes, and the induction of cachexia.

The Protein Data Bank website provides the crystal structure of TNF-α searchable by 6RMJ (Valentinis, B., et al., Int. J. Mol. Sci., 2019, 20); 5UUI (Carrington et al., Biophys J., 2017, 113 371-380); 600Y, 600Z and 60PO (O'Connell, J., et al., Nat. Commun., 2019, 10 5795-5795); and 5TSW (Cha, S. S., J Biol Chem., 1998, 273 2153-2160); as well as the crystal structure of TNF-α bound to various compounds searchable by 5YOY (Ono et al., Protein Sci., 2018, 27 1038-1046); 2AZ5 (He., M. M., et al., Science, 2005, 310: 1022-1025); 5WUX (Lee, J. U., Int J Mol Sci., 2017, 18); 5MU8 (Blevitt et al., J Med Chem., 2017, 60 3511-3517); 4Y60 (Feldman J.

L., et al., Biochemistry, 2015, 54 3037-3050); 3WD5 (Hu, S., et al., J Biol Chem, 2013, 288 27059-27067); and 4G3Y (Liang, S. Y., J Biol Chem., 2013, 288 13799-13807).

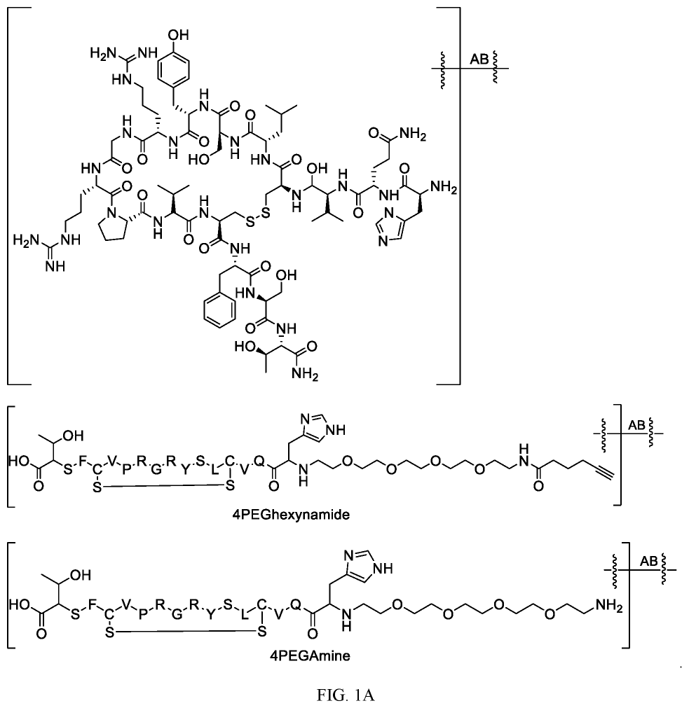

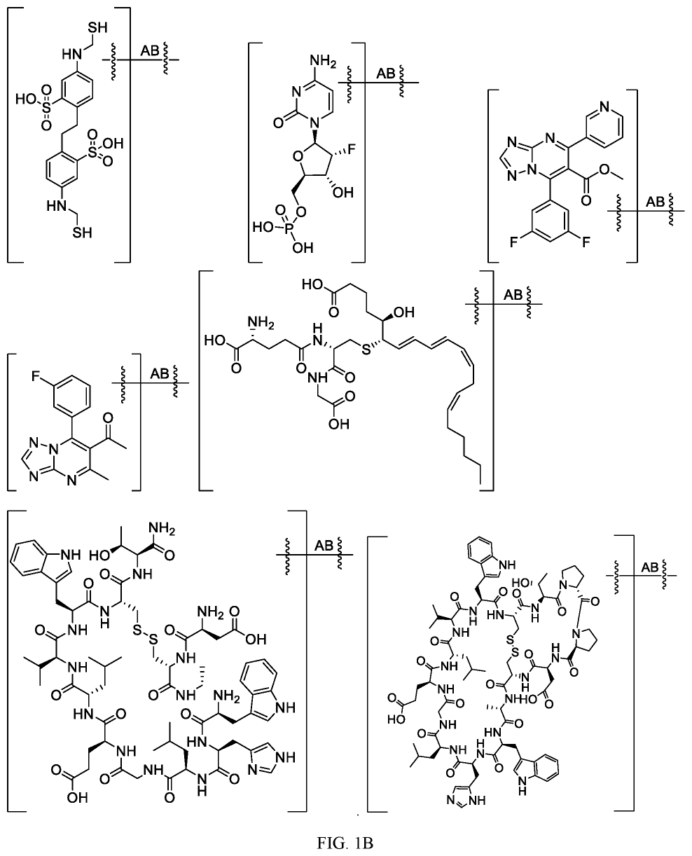

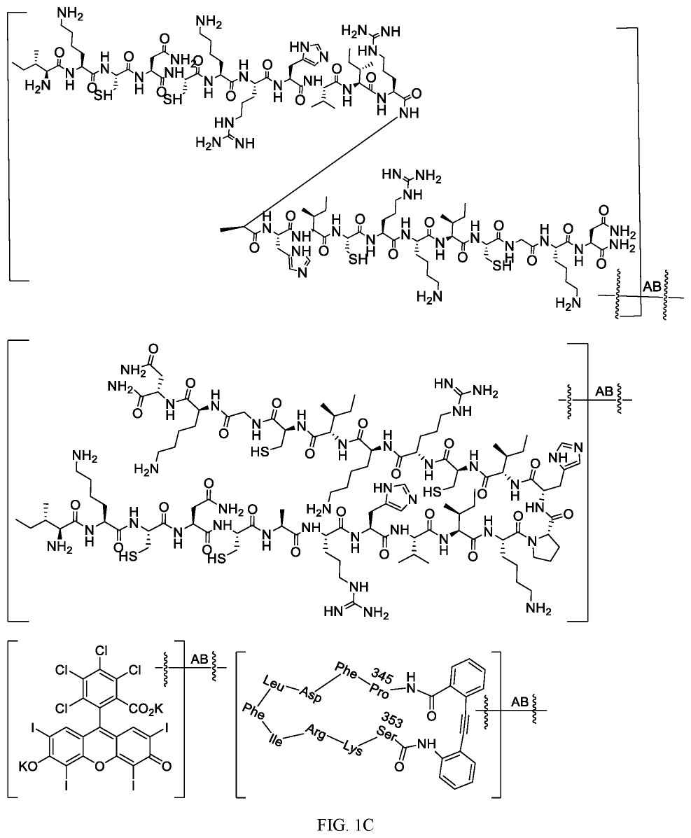

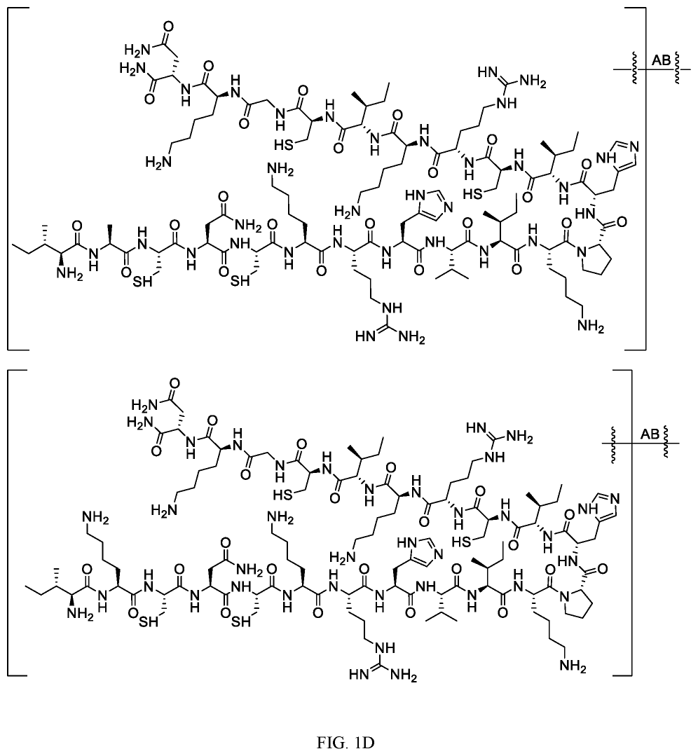

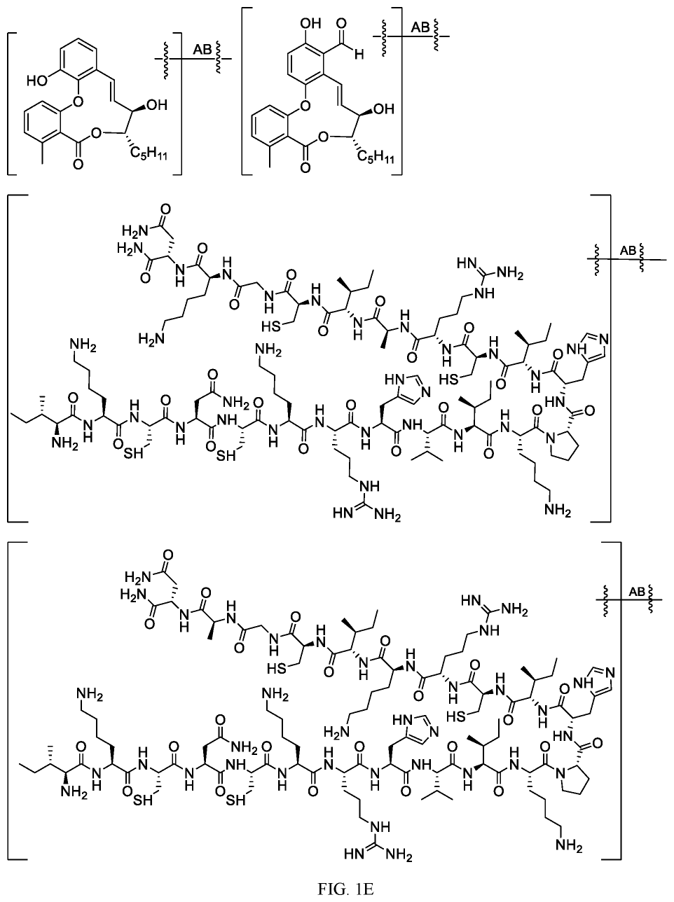

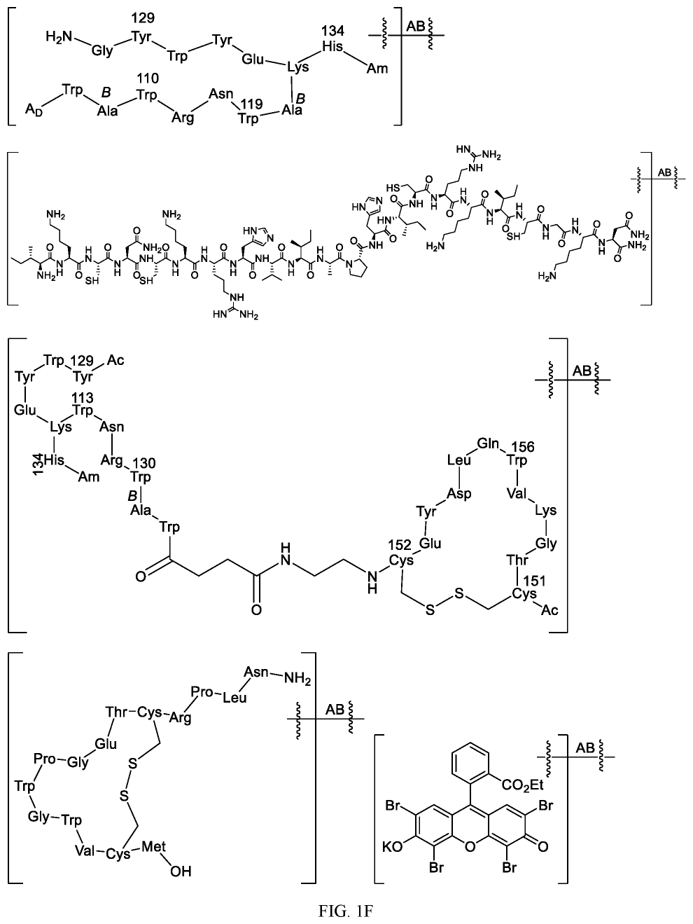

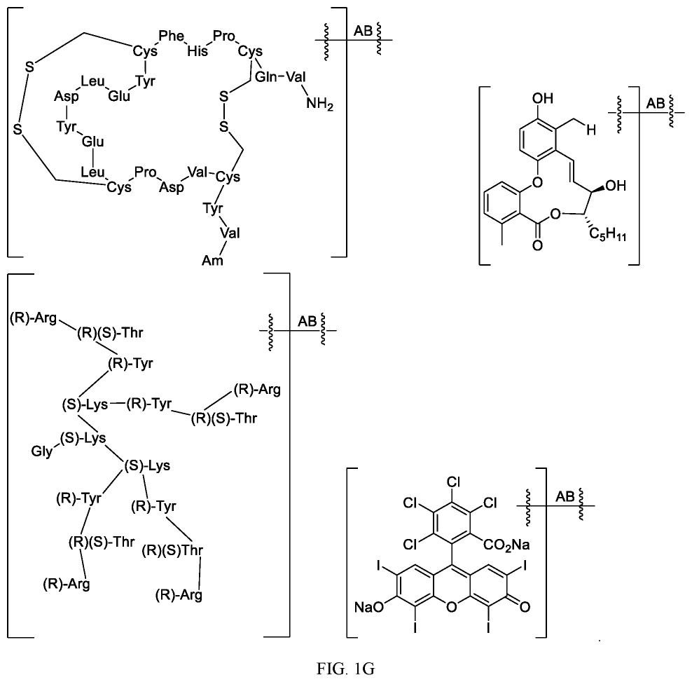

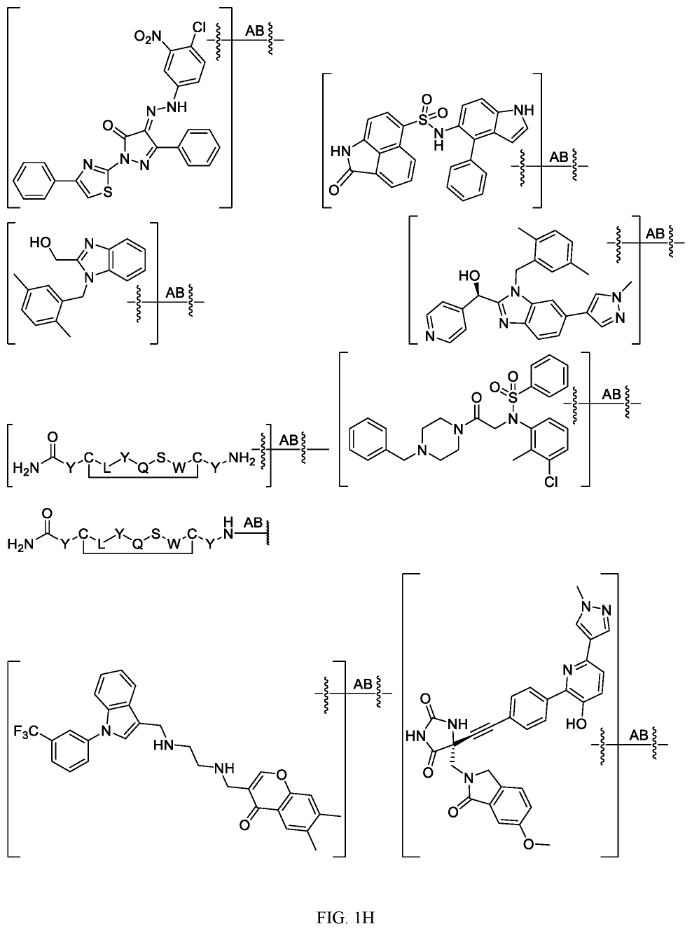

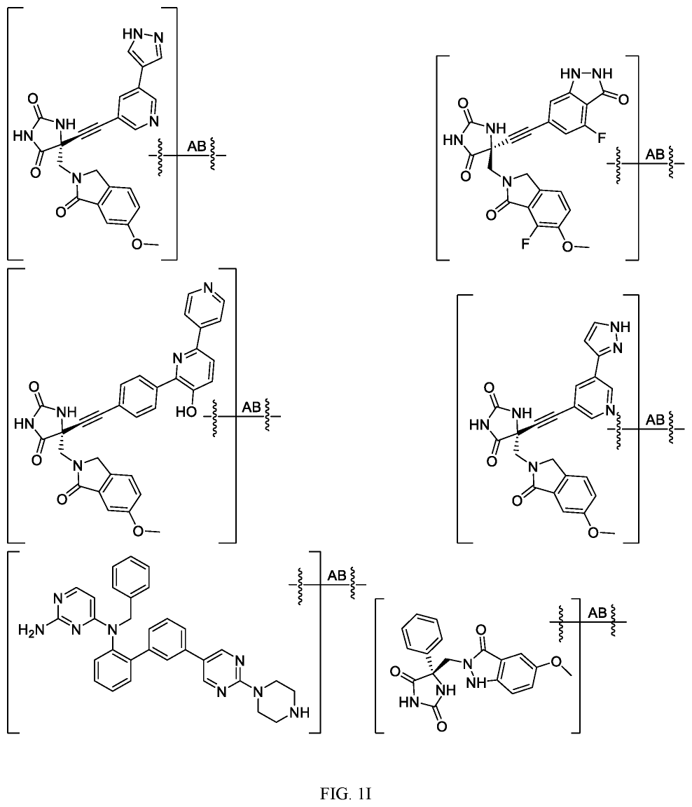

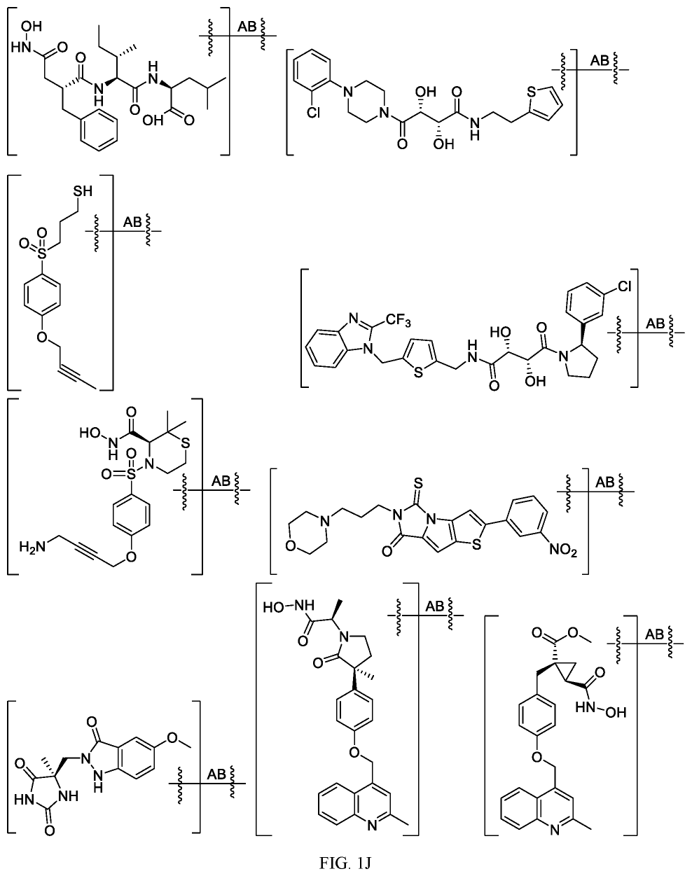

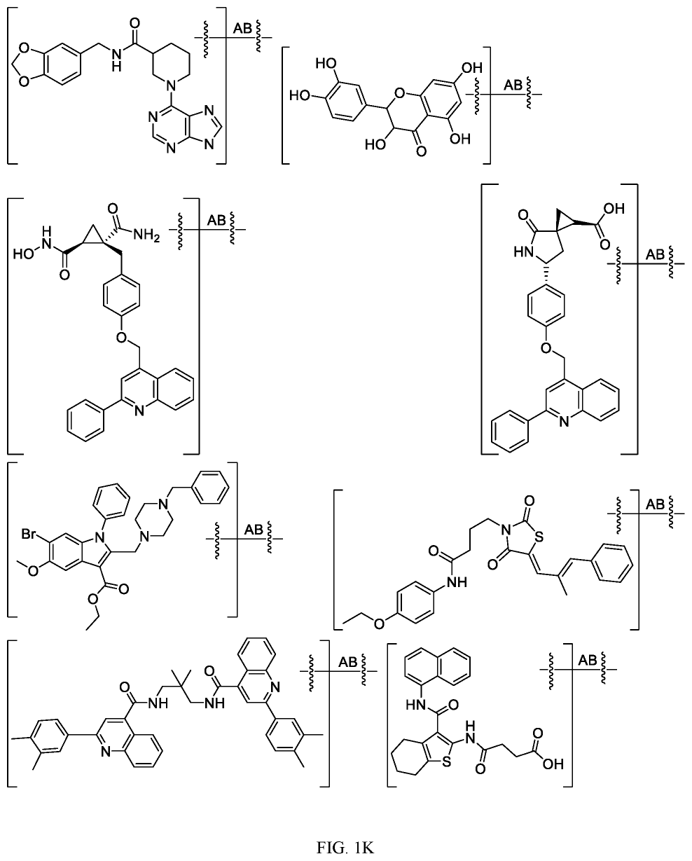

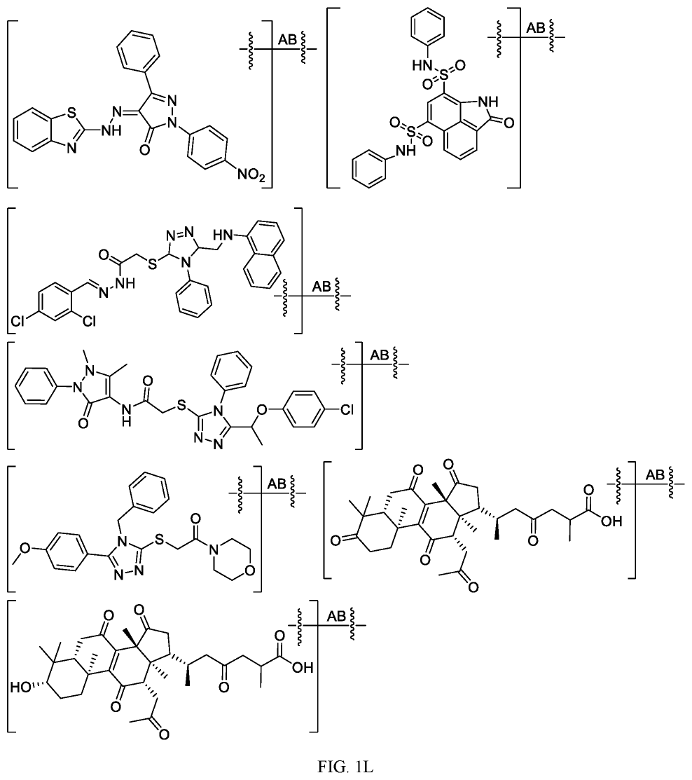

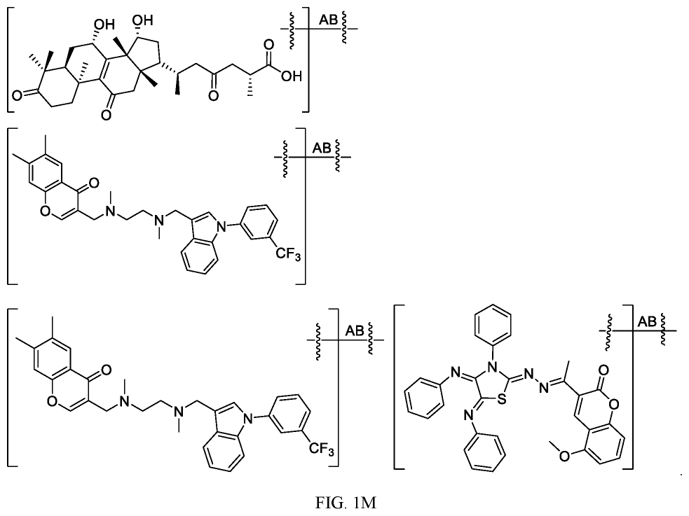

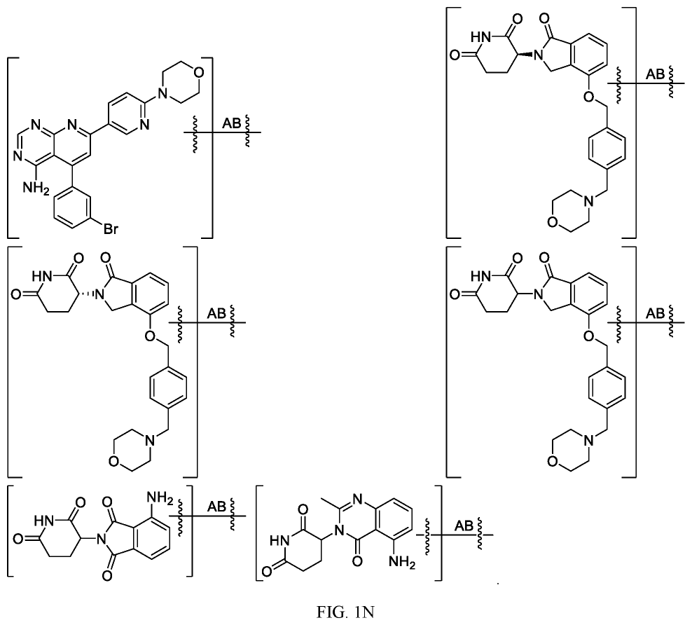

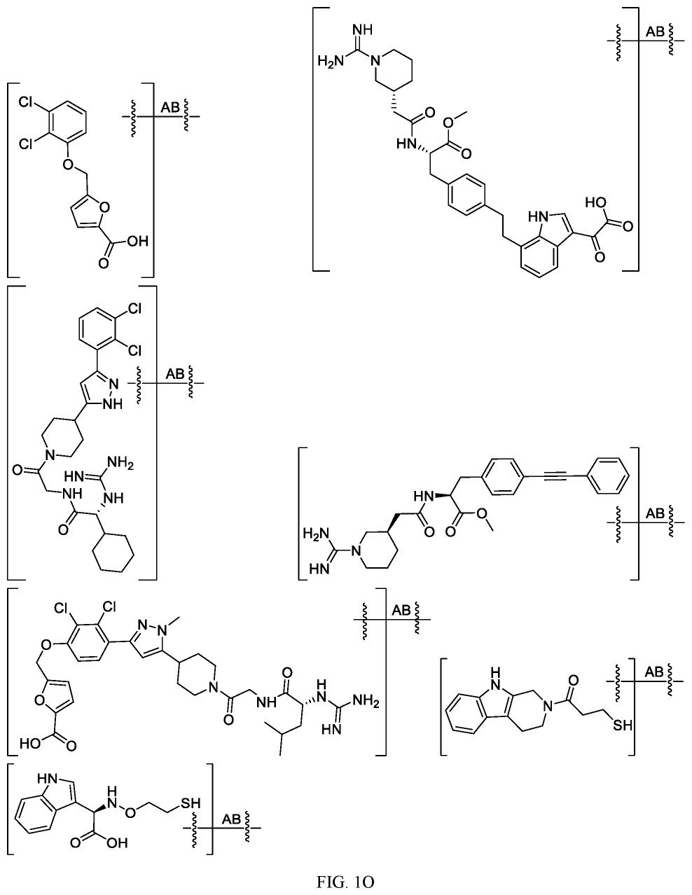

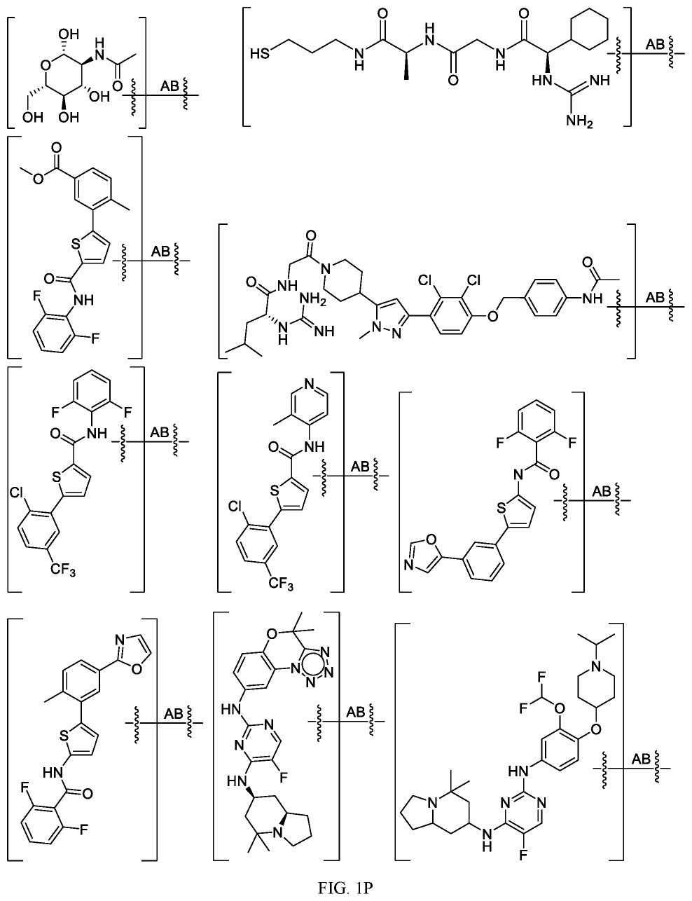

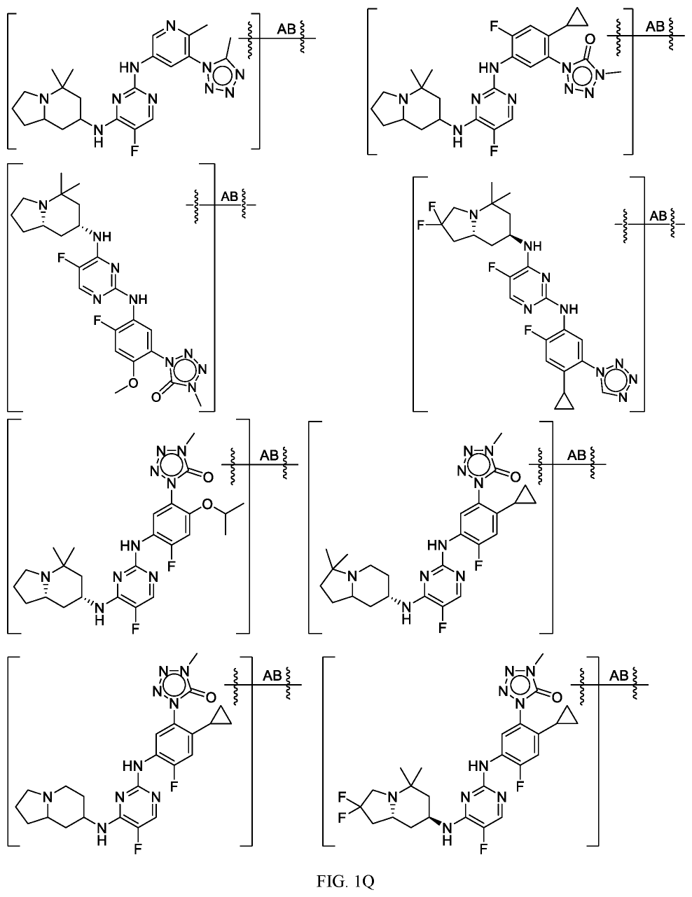

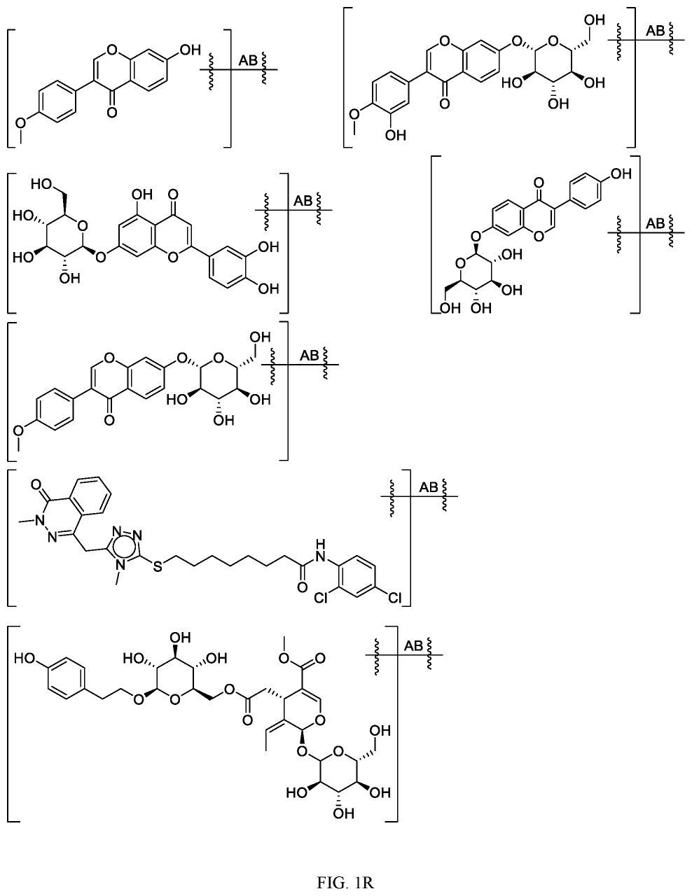

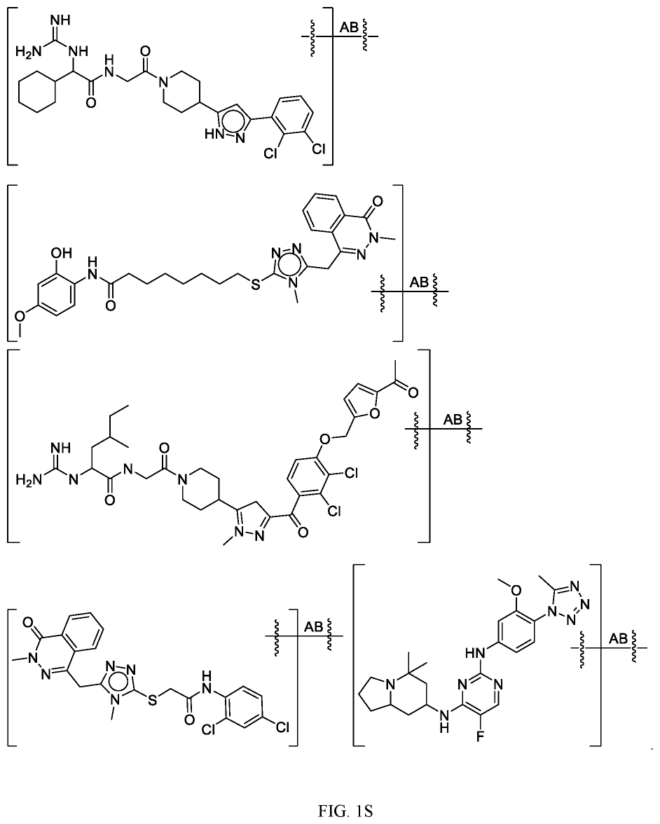

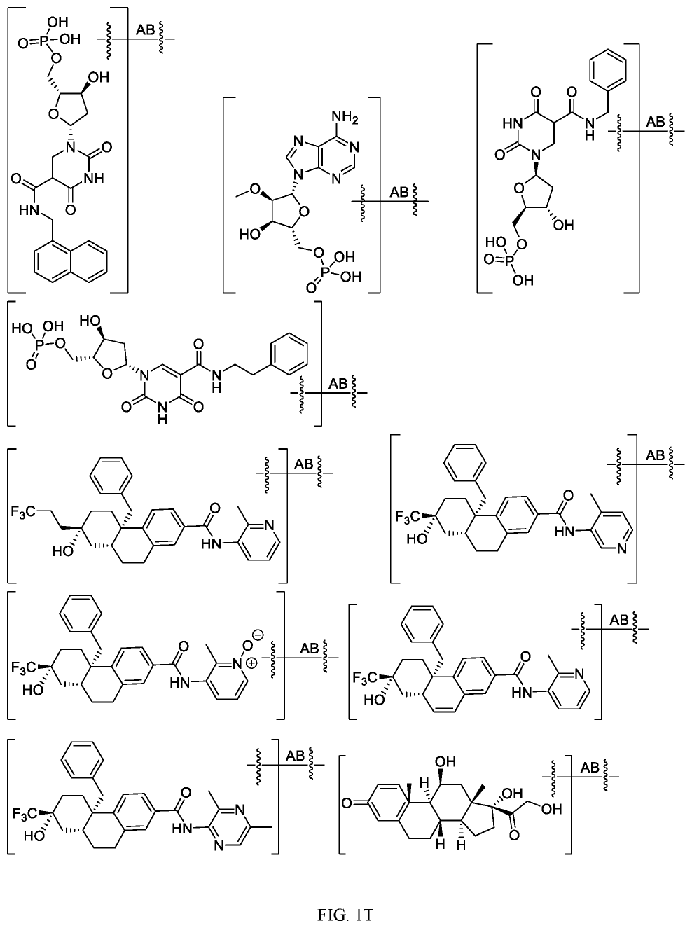

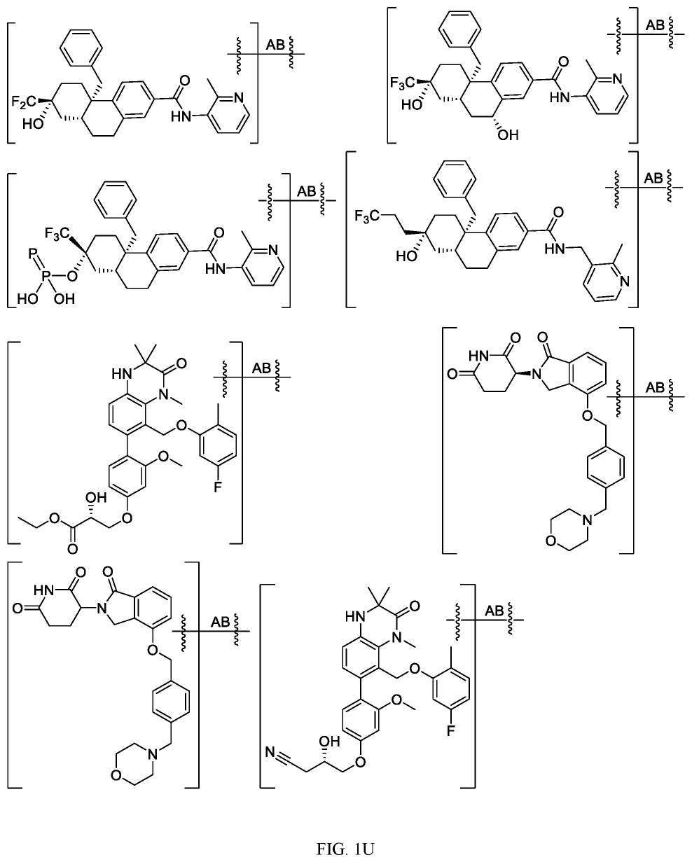

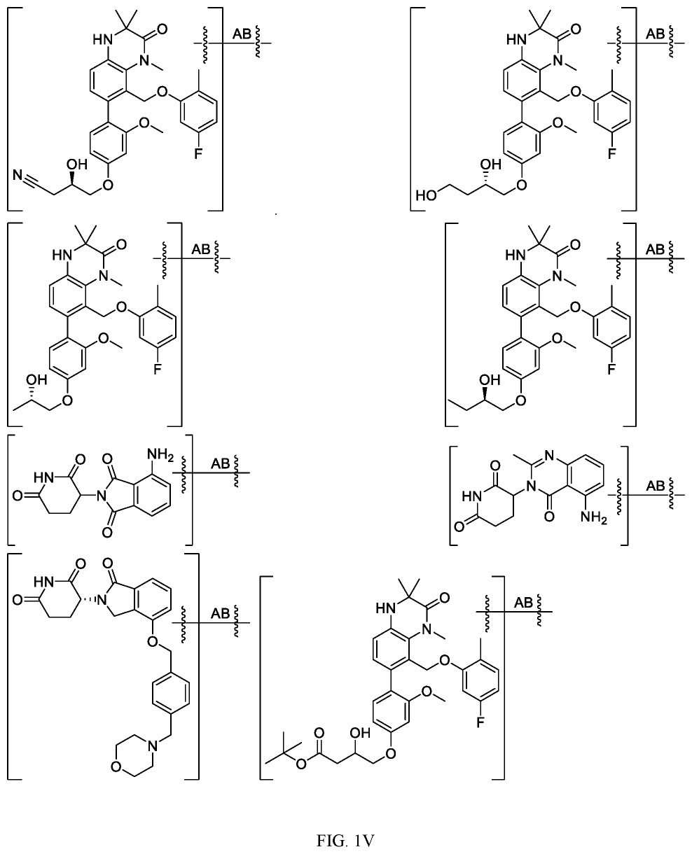

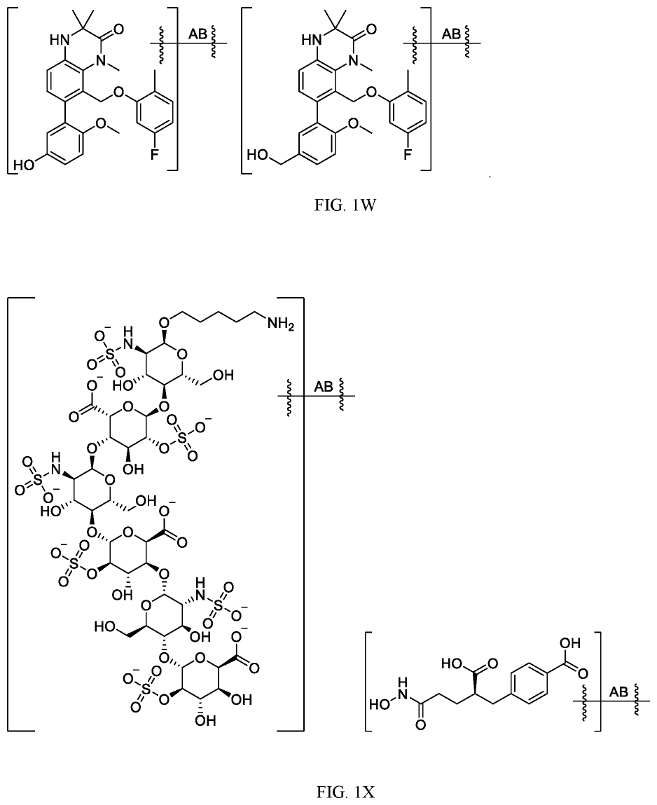

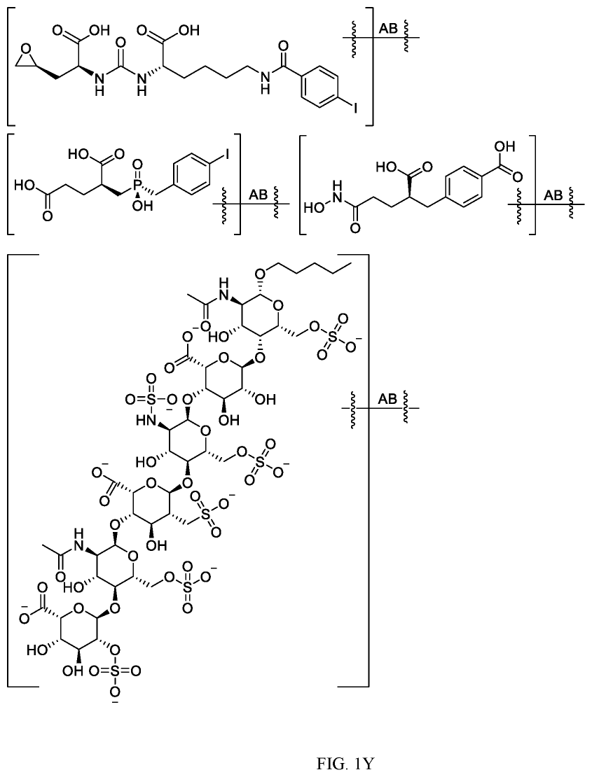

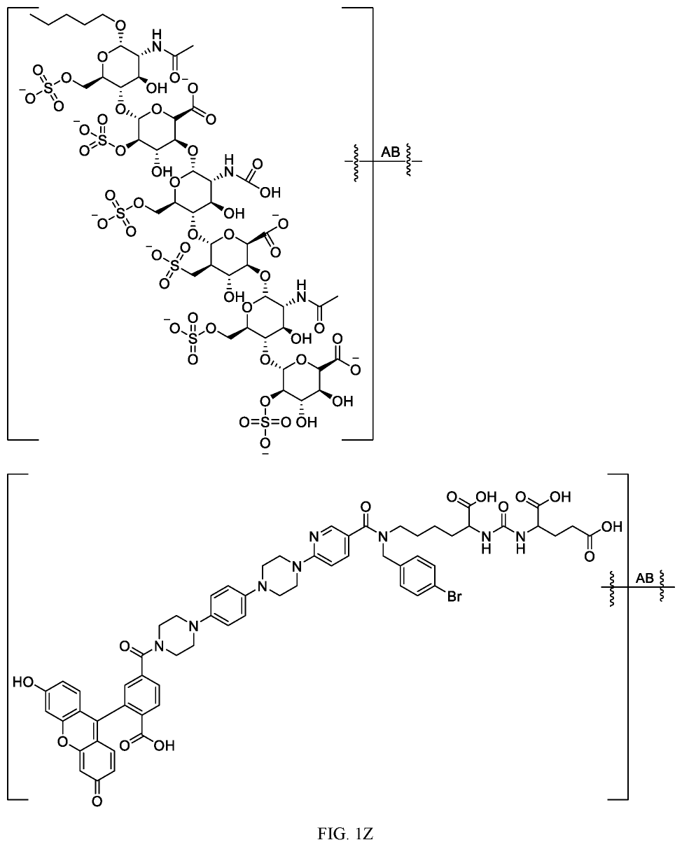

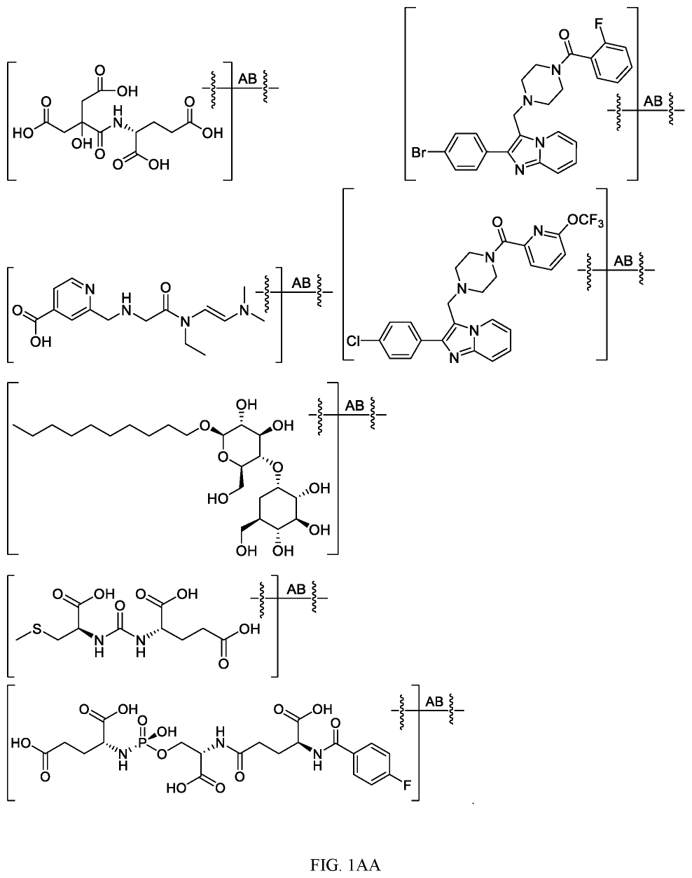

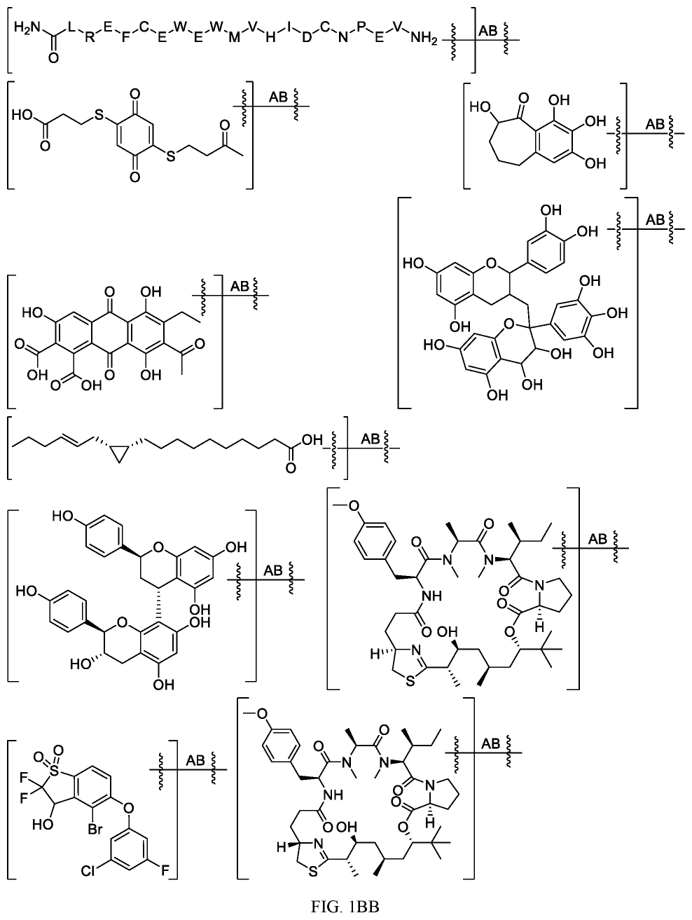

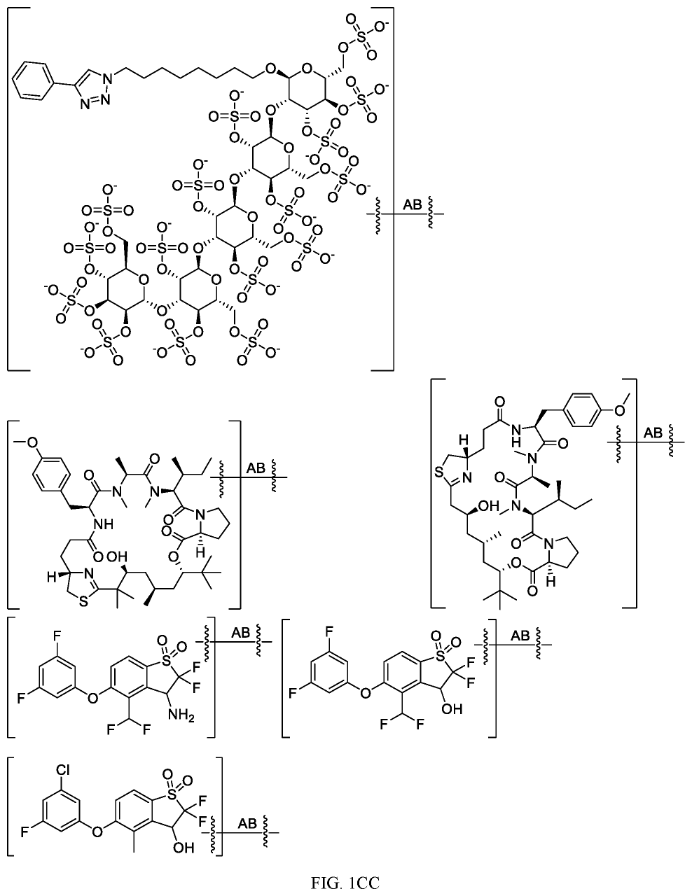

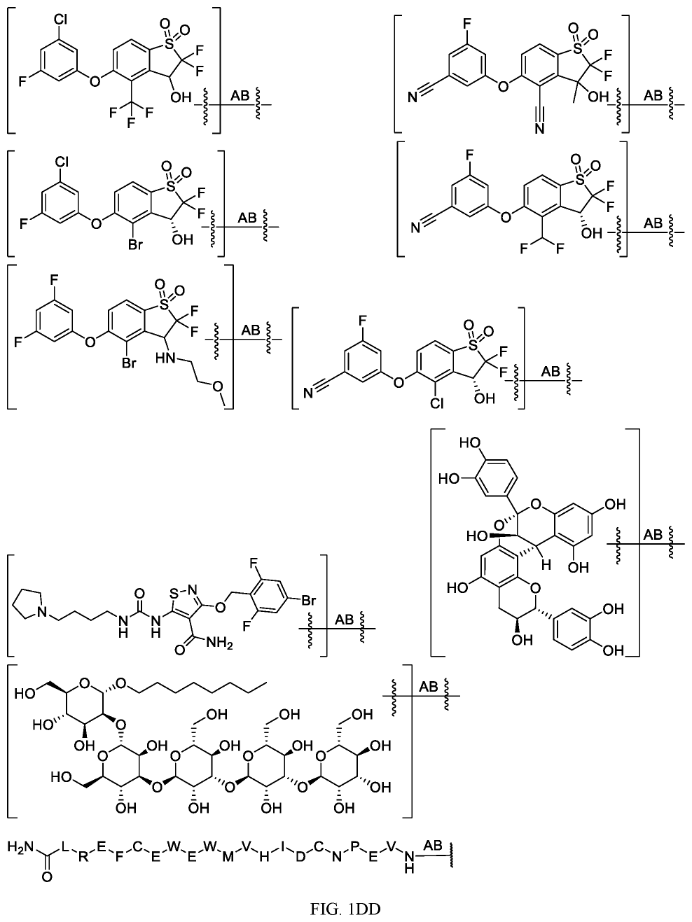

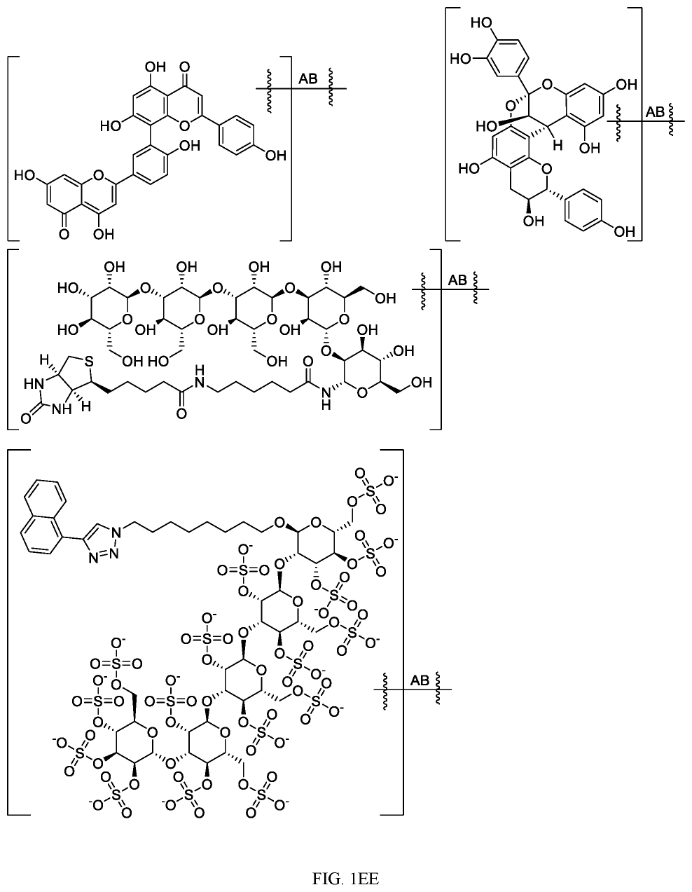

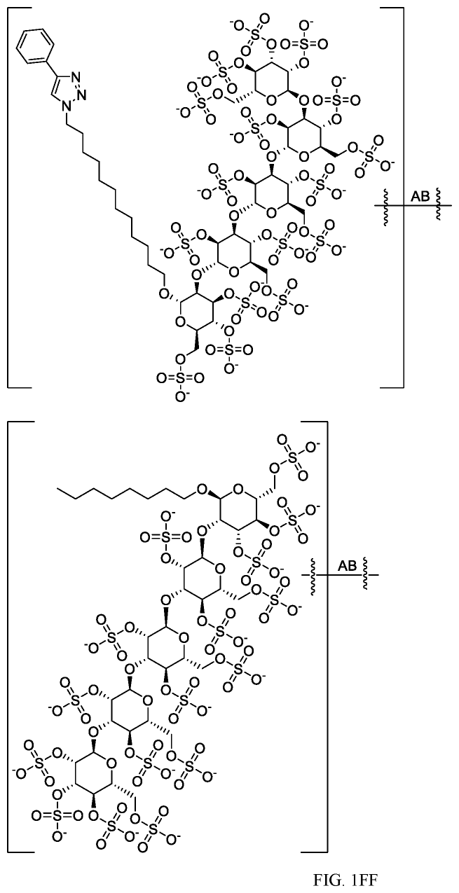

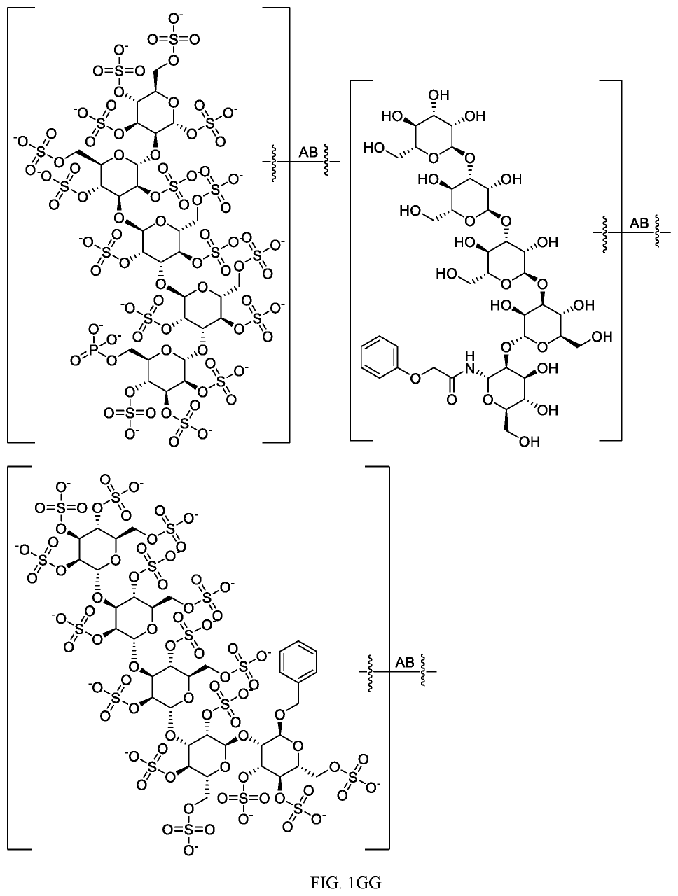

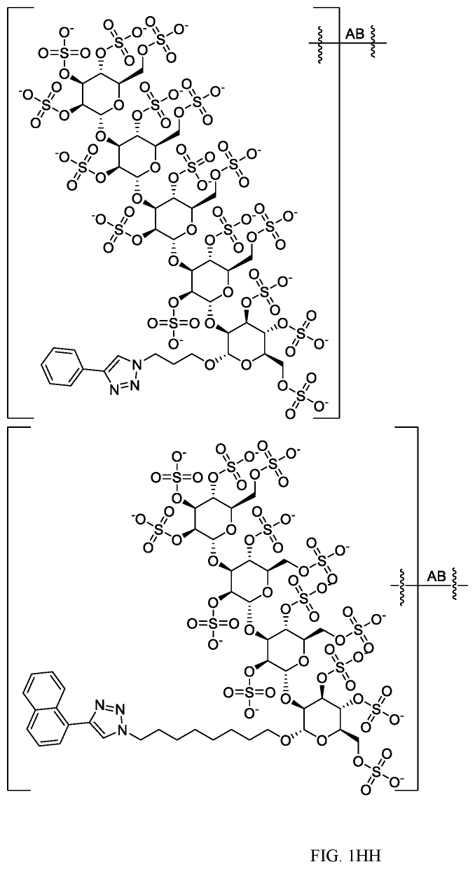

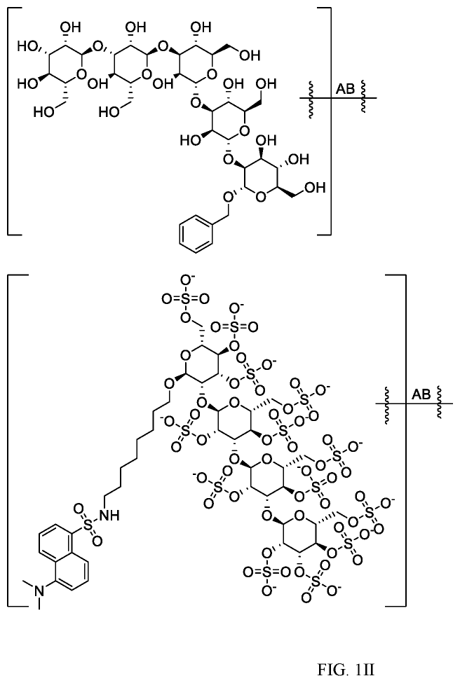

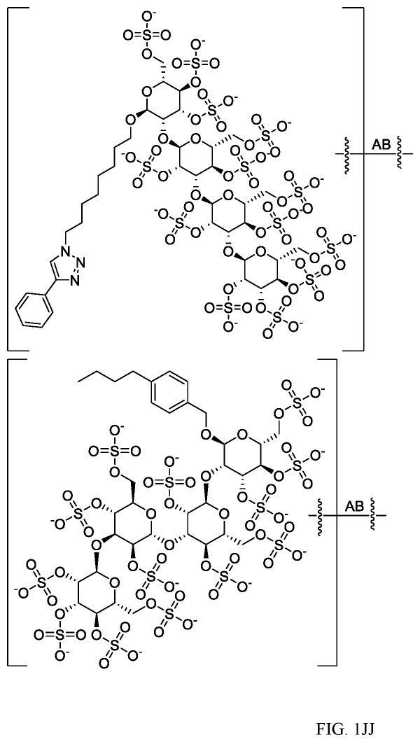

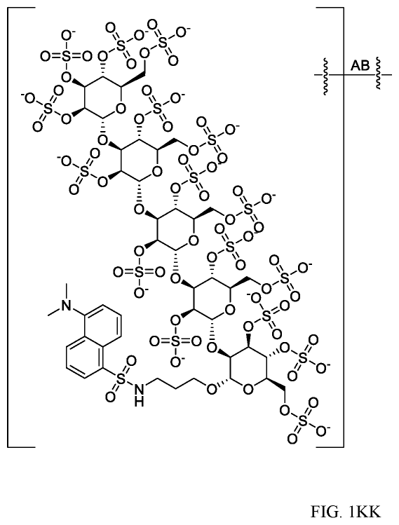

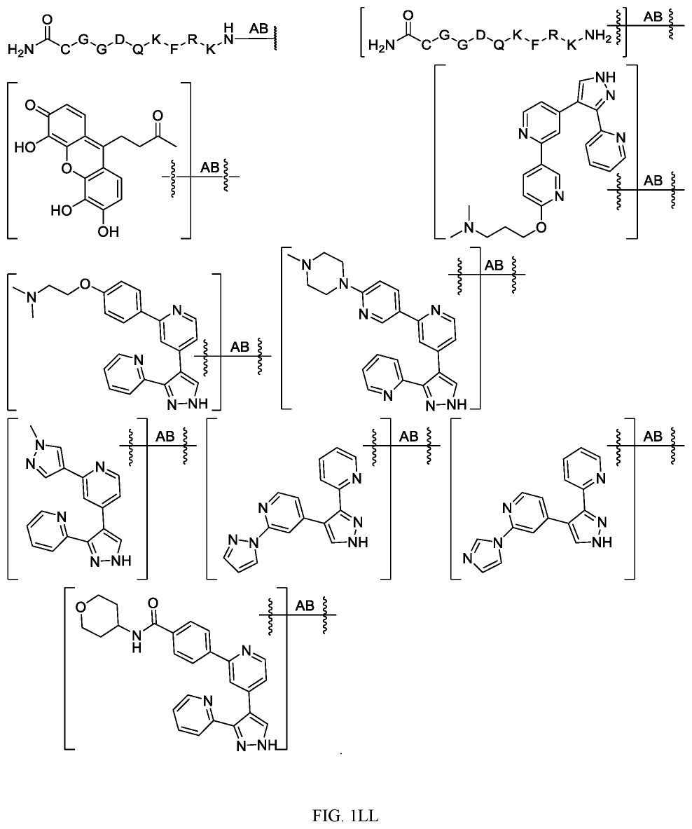

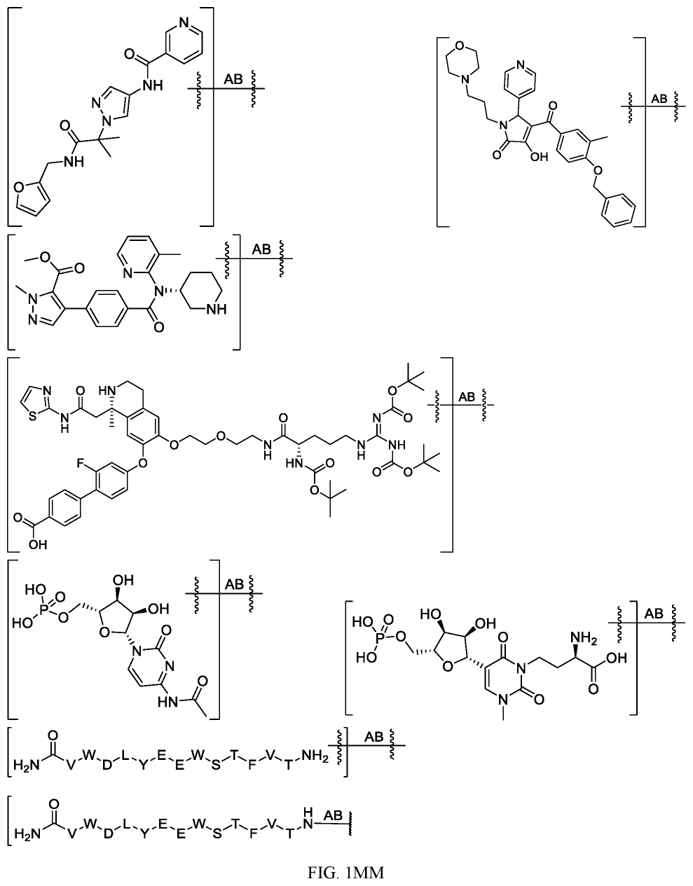

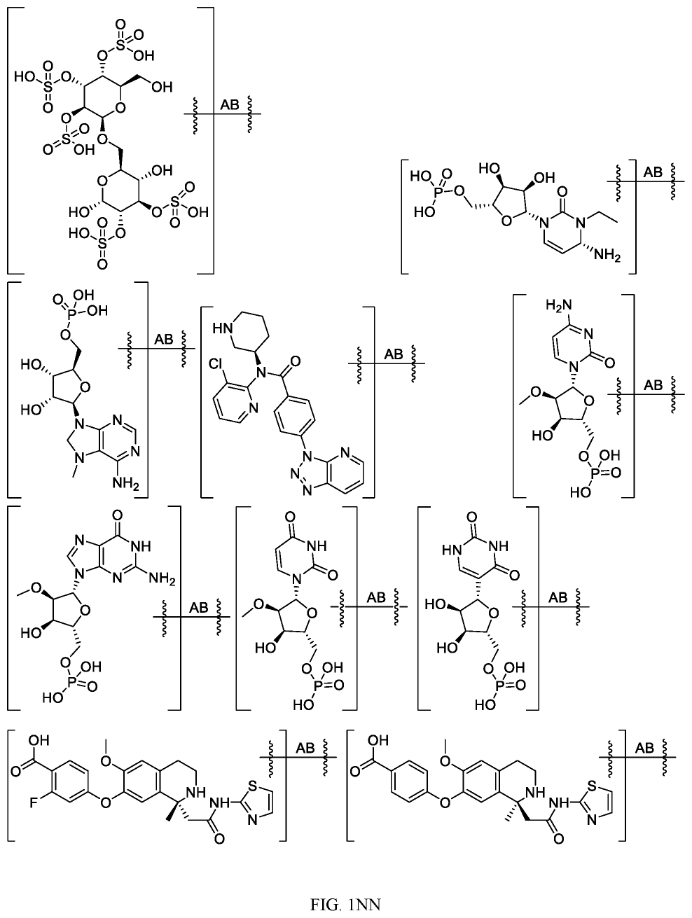

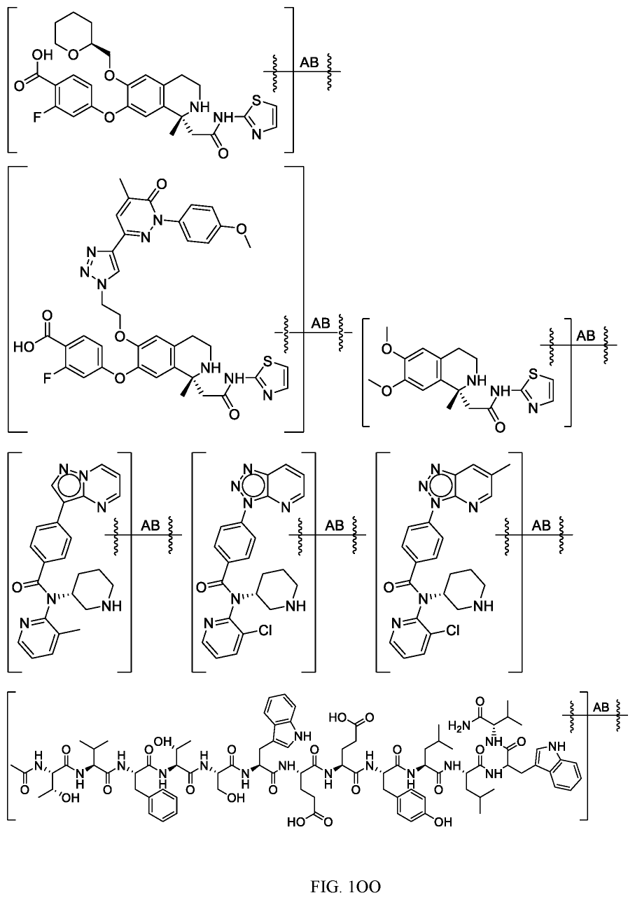

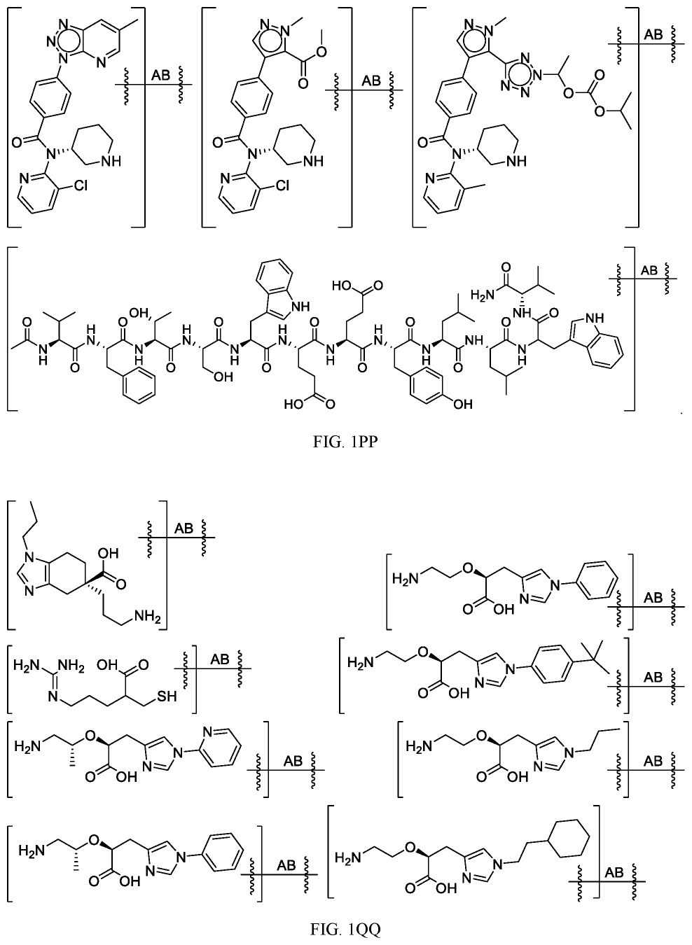

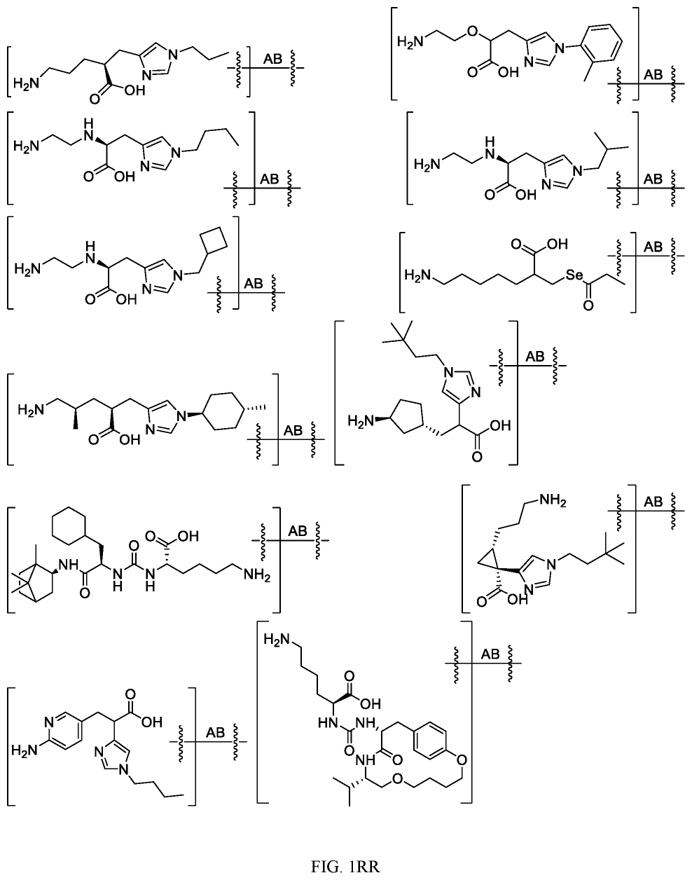

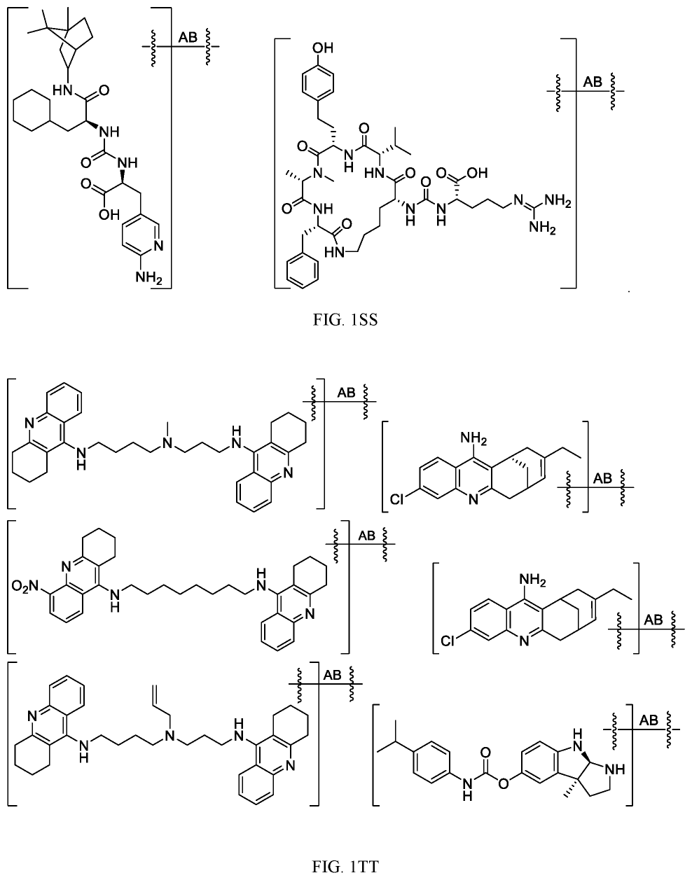

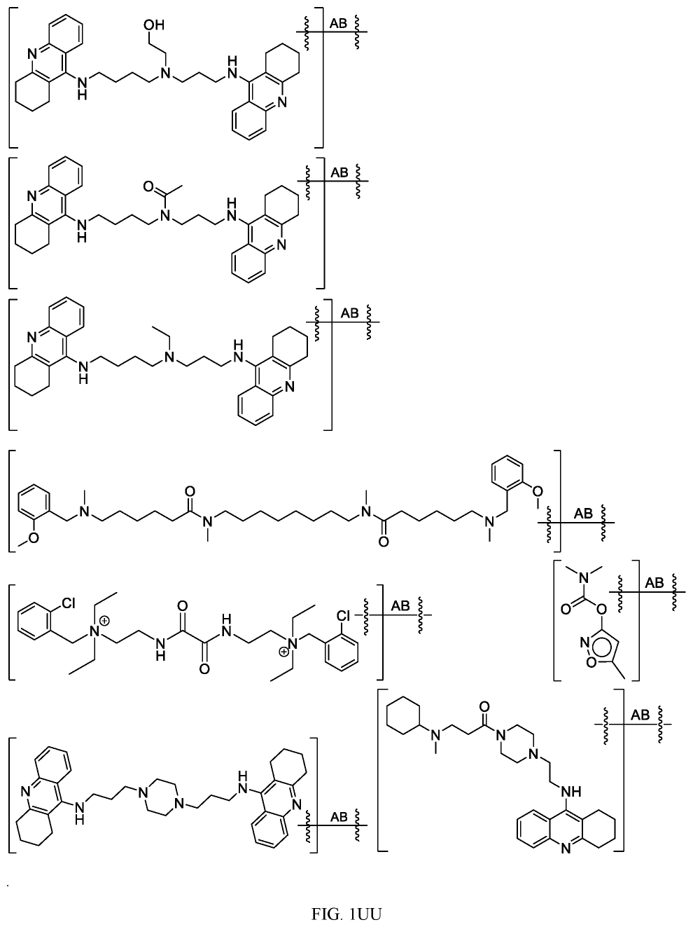

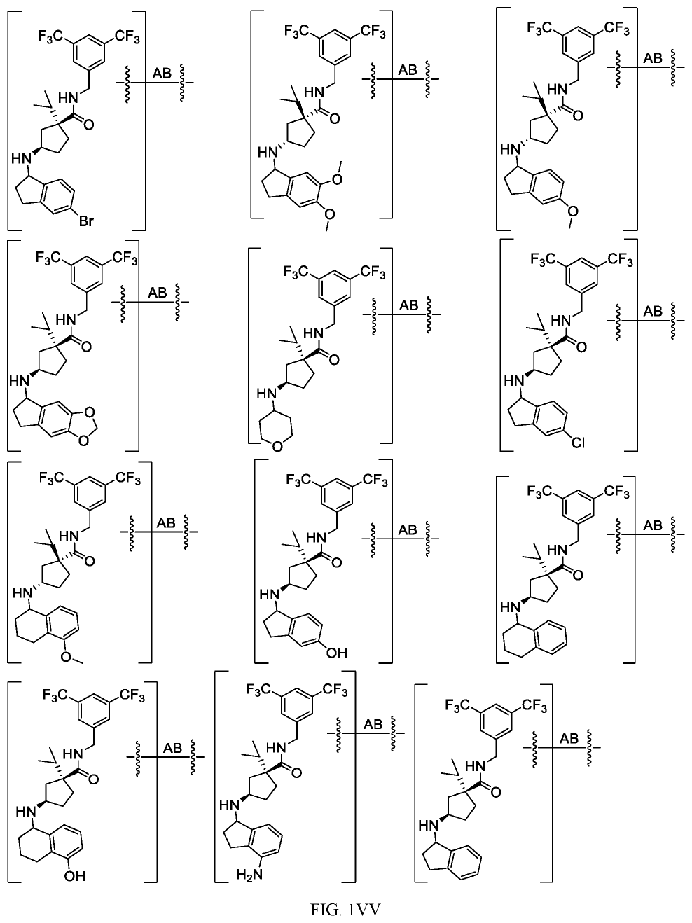

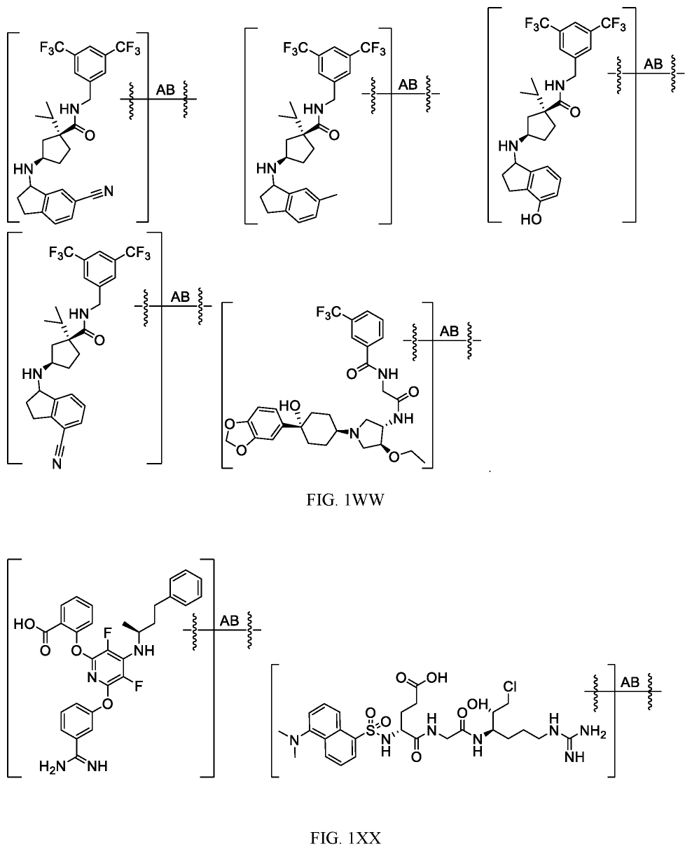

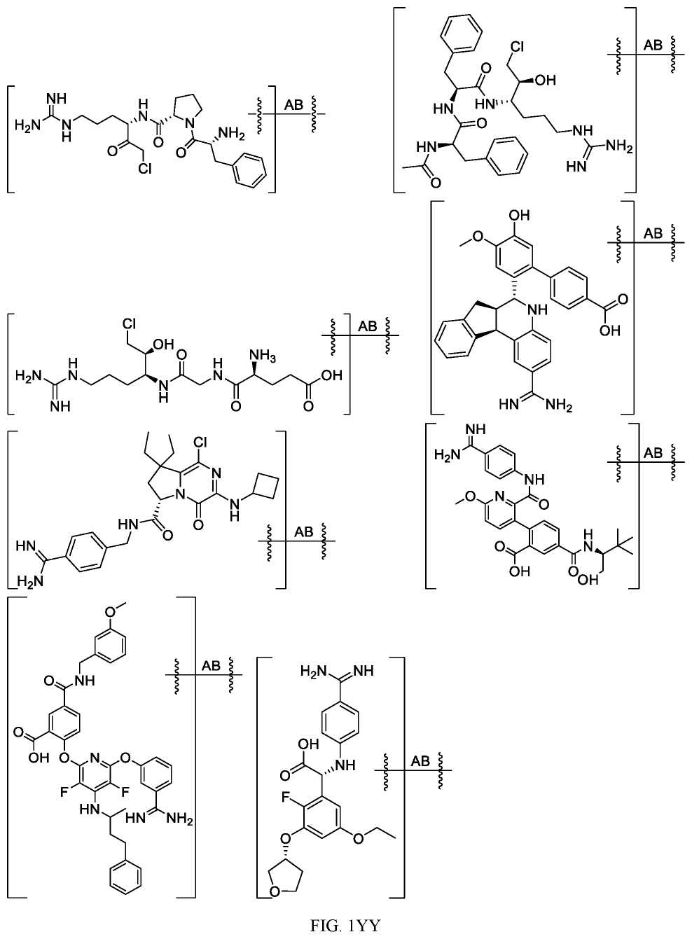

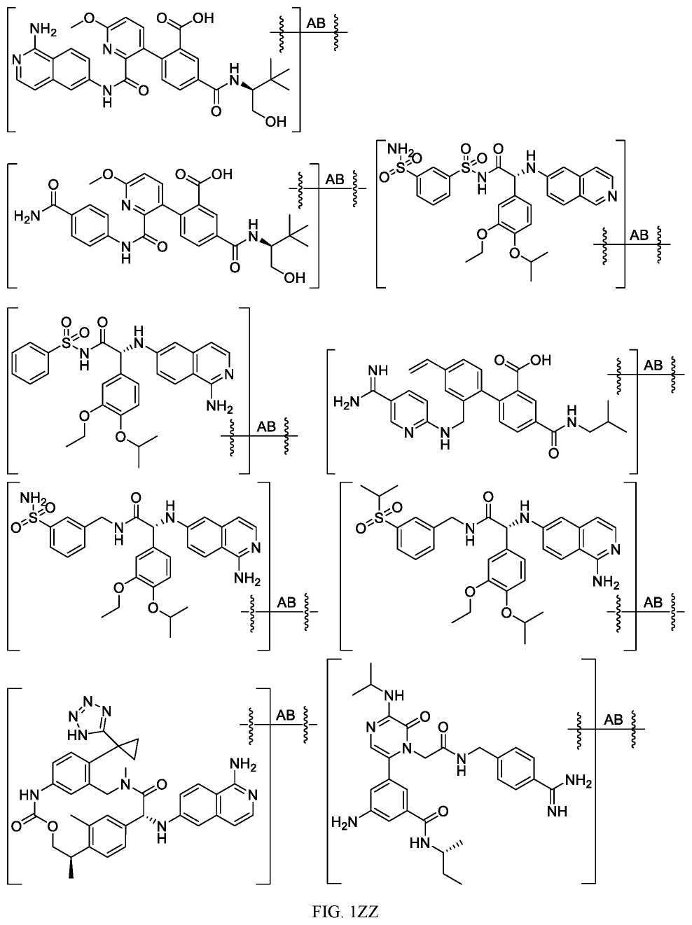

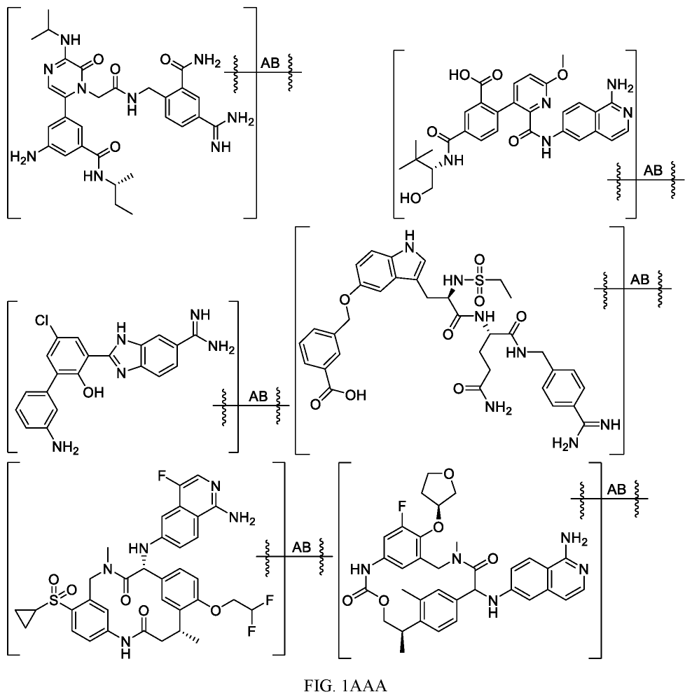

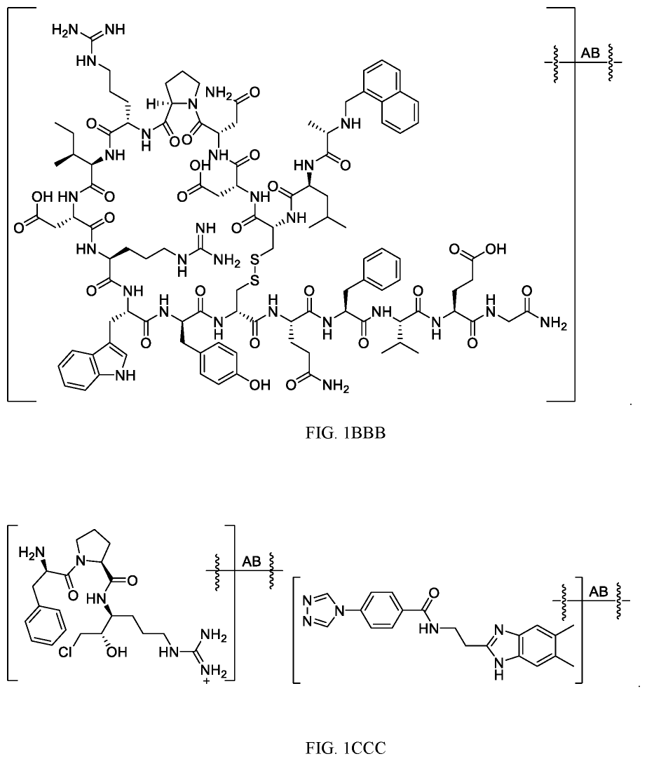

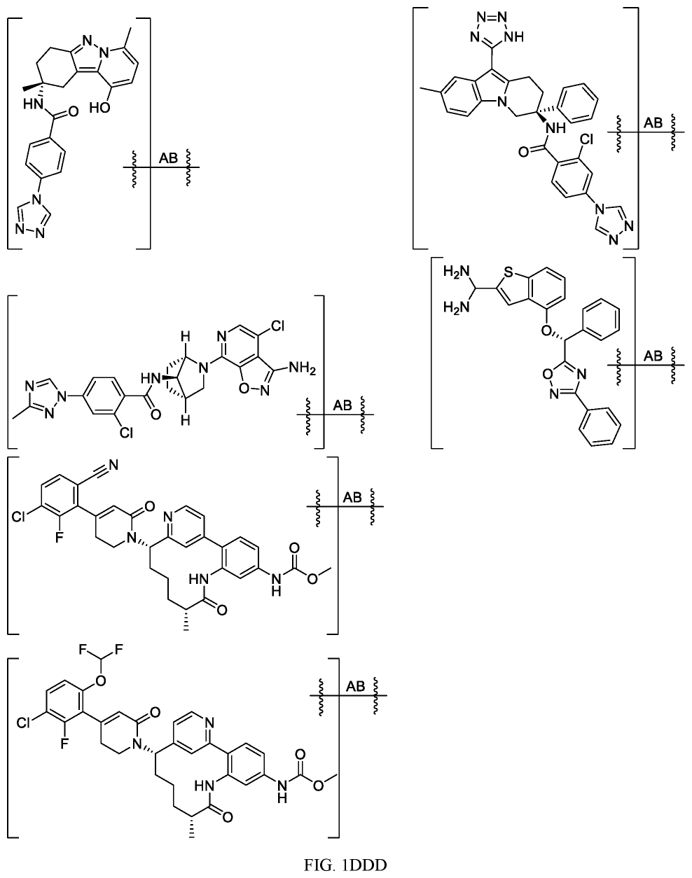

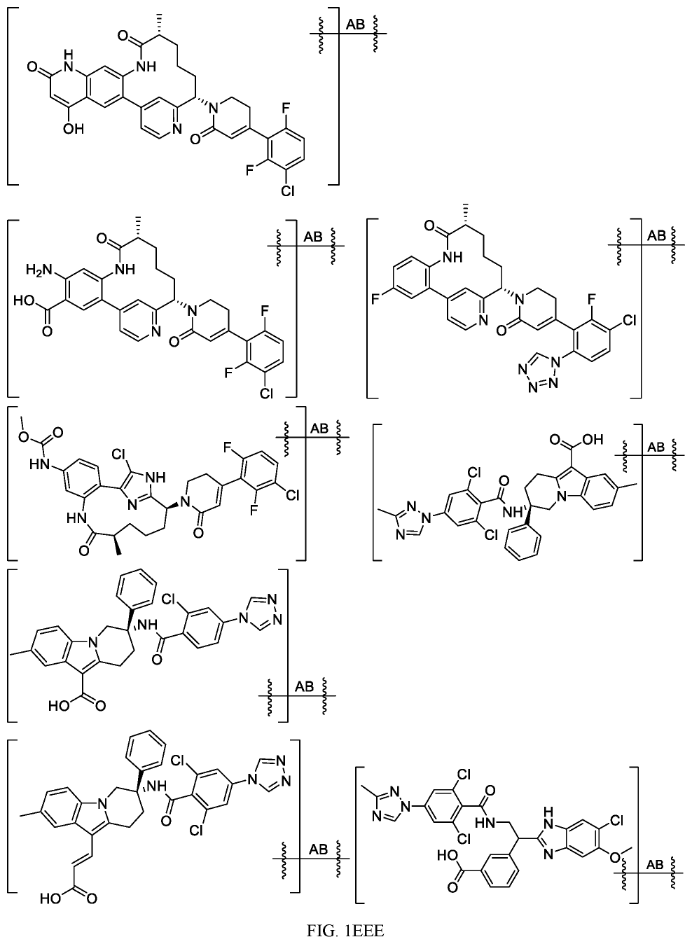

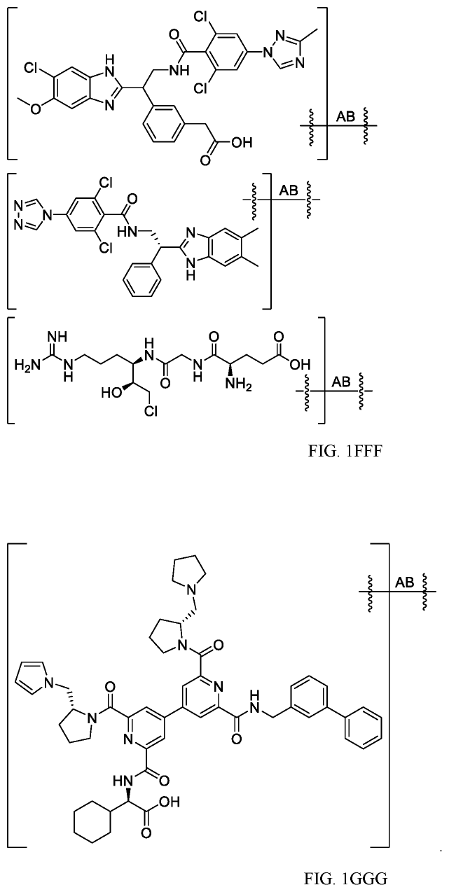

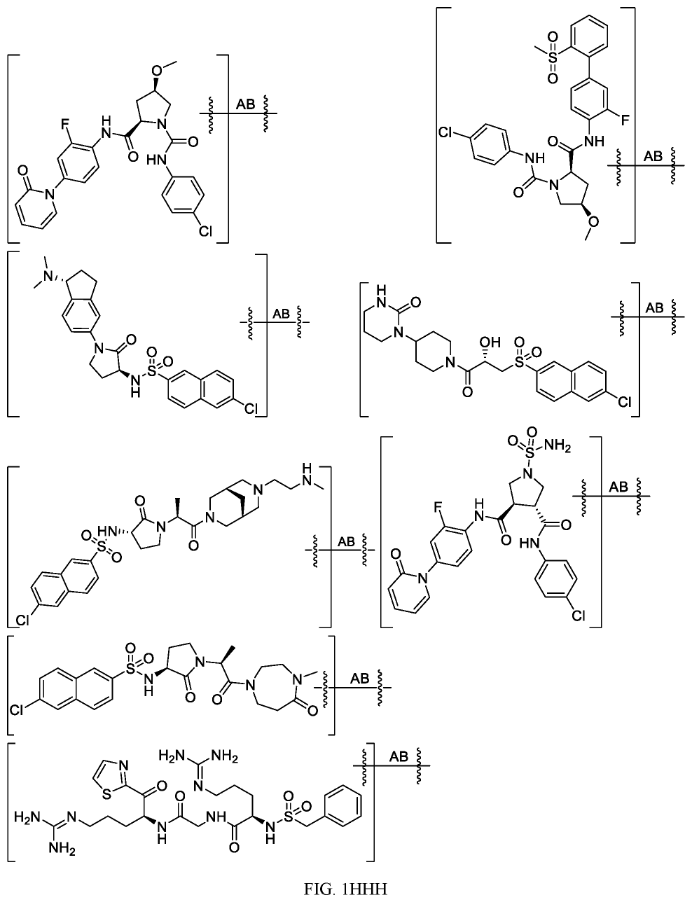

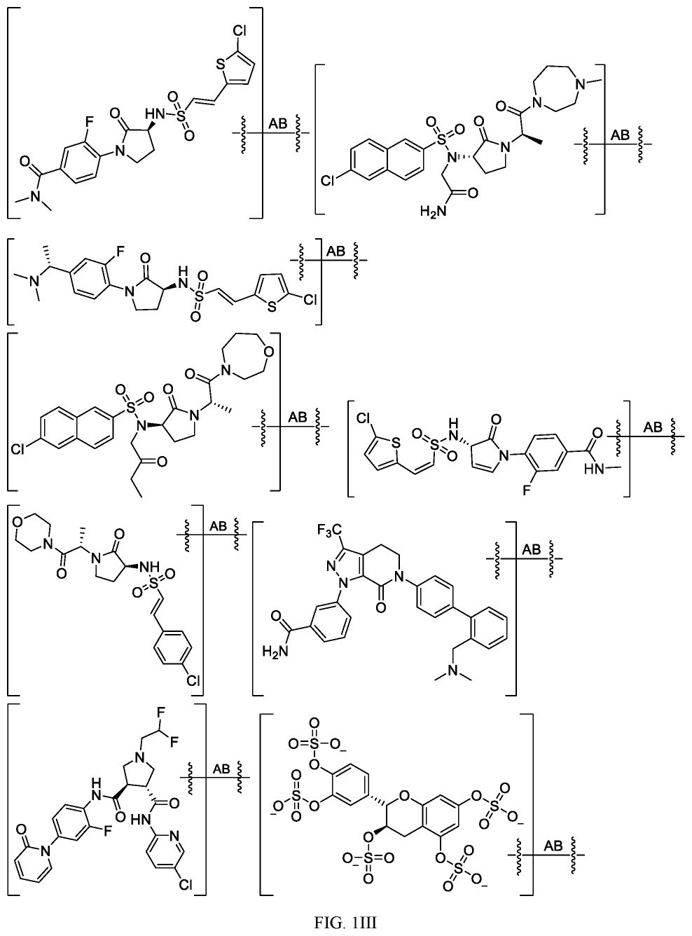

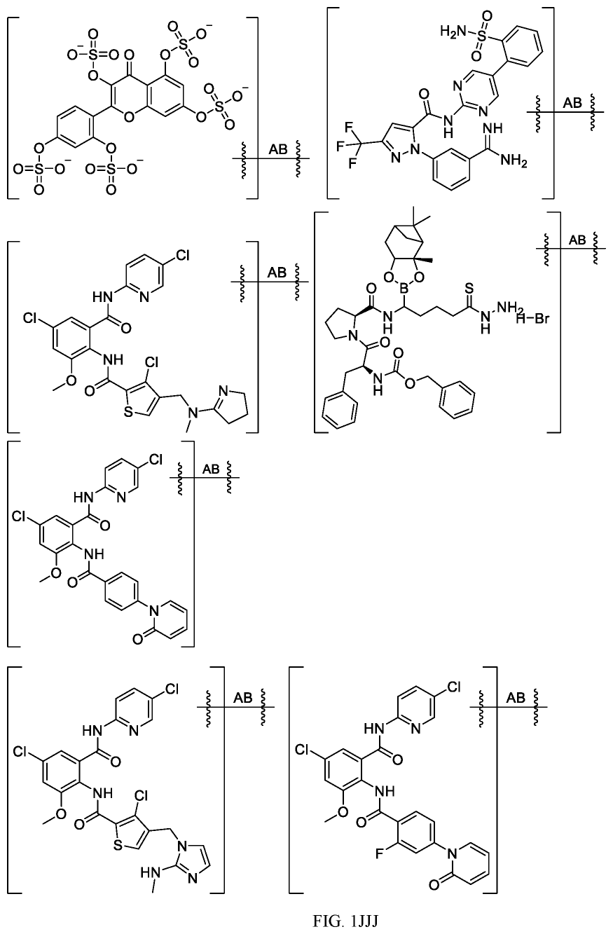

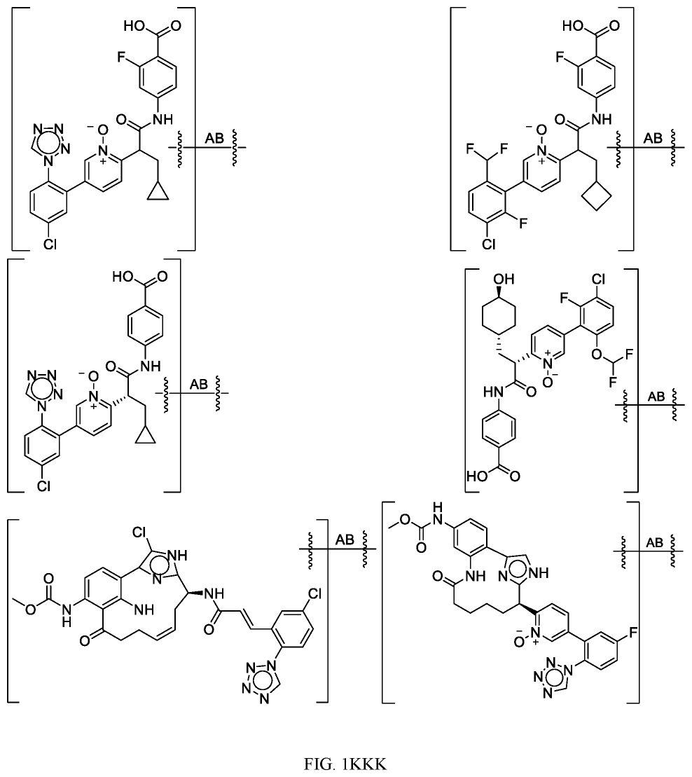

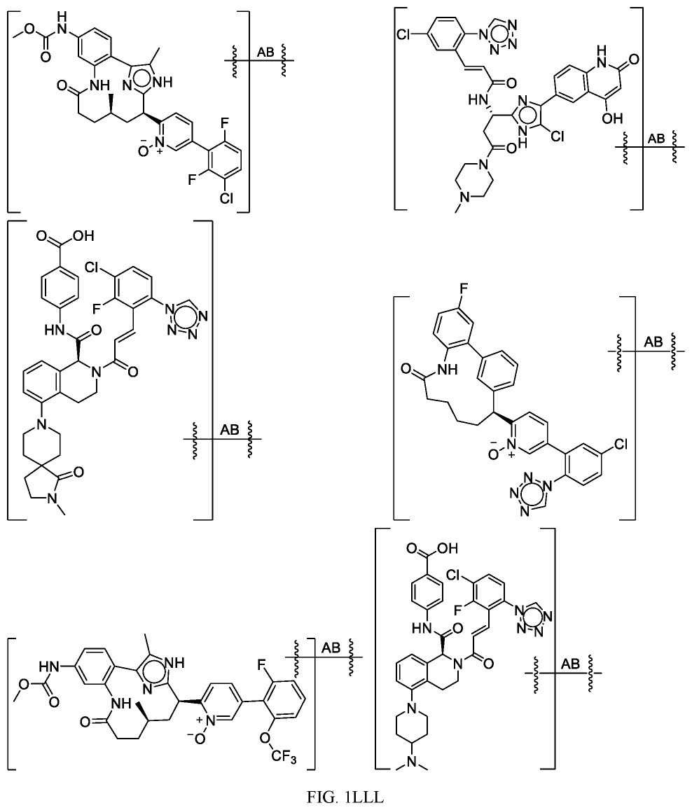

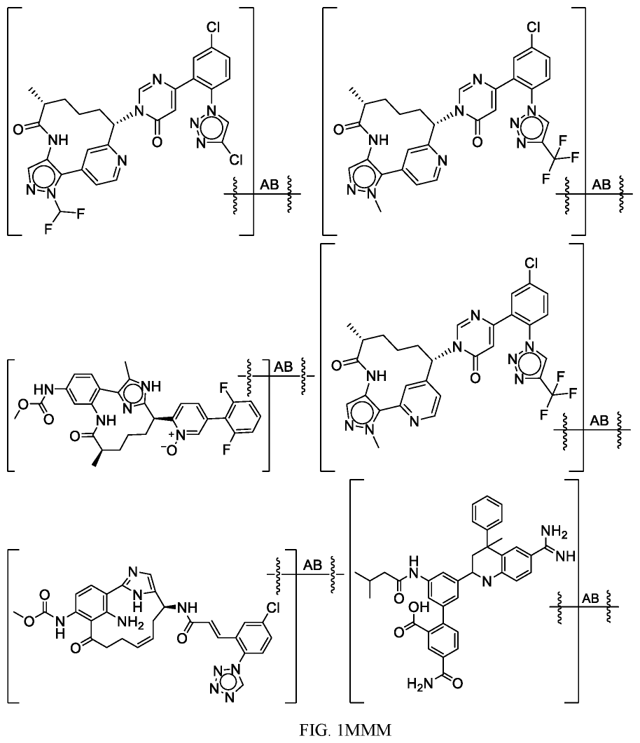

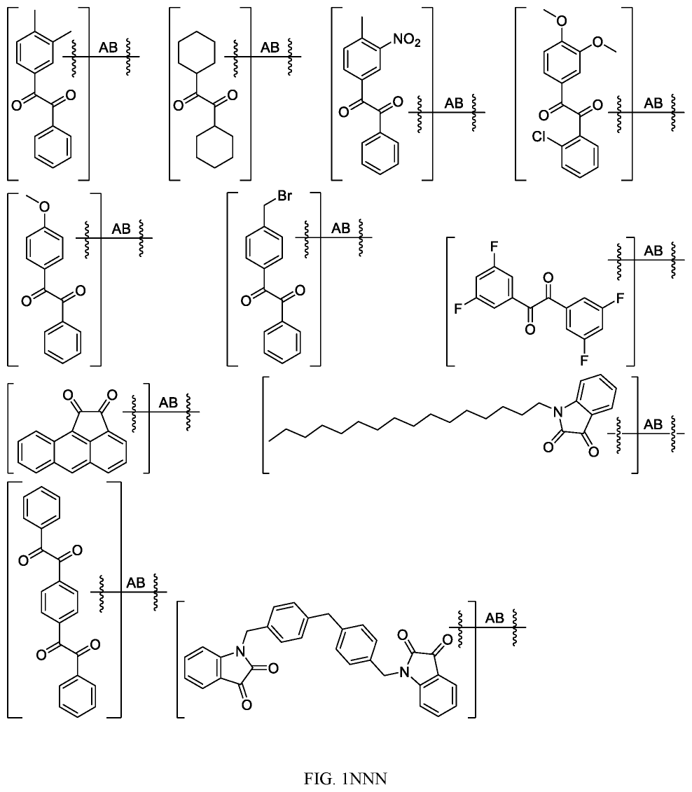

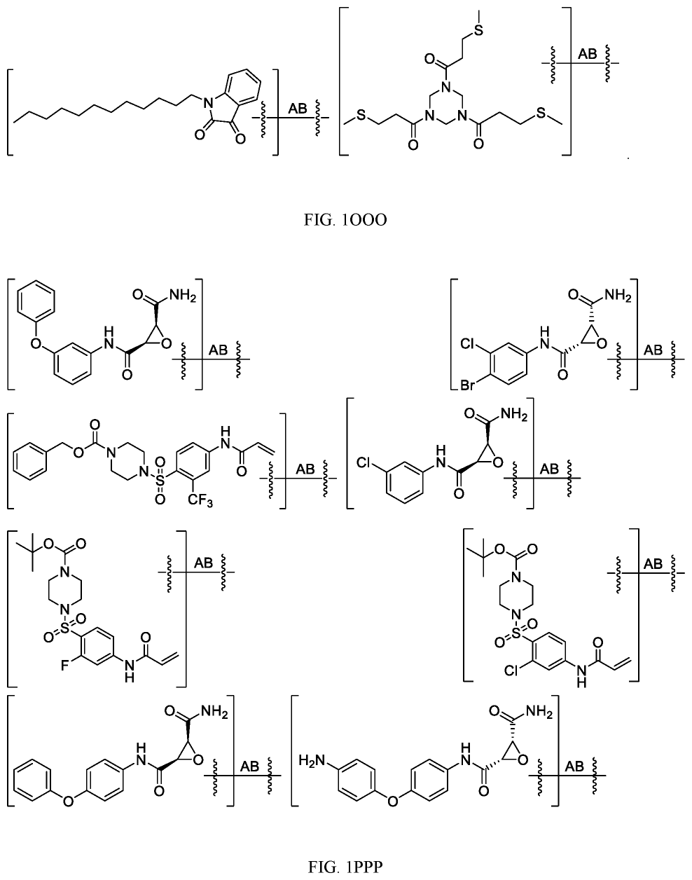

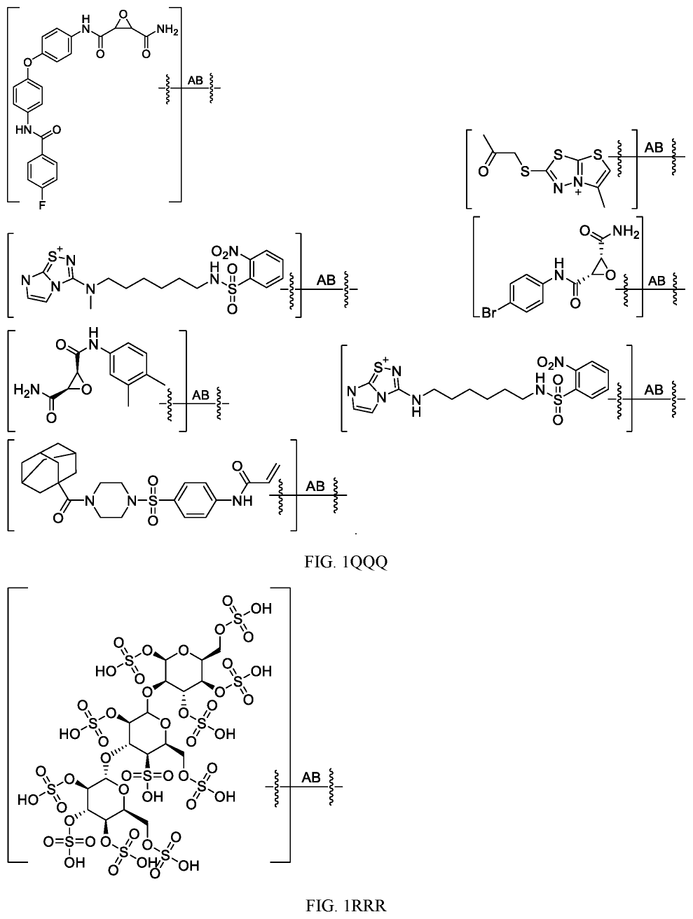

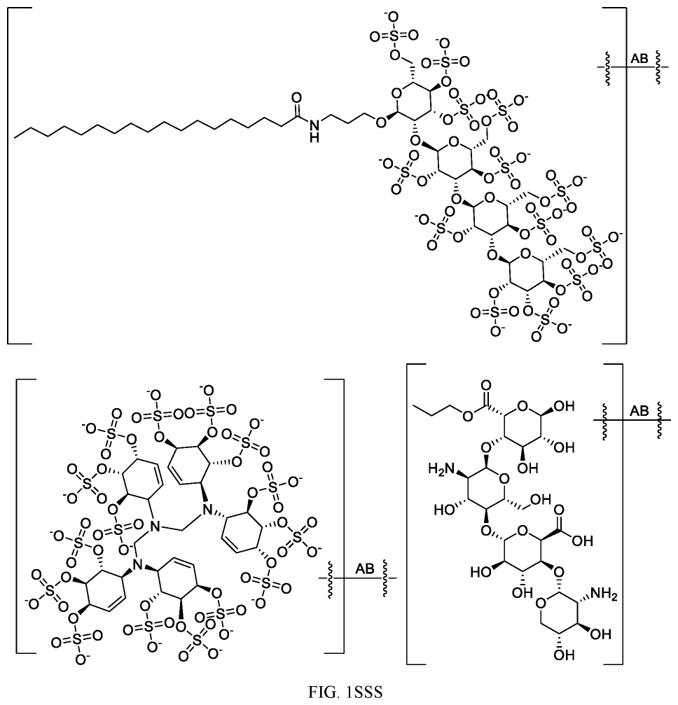

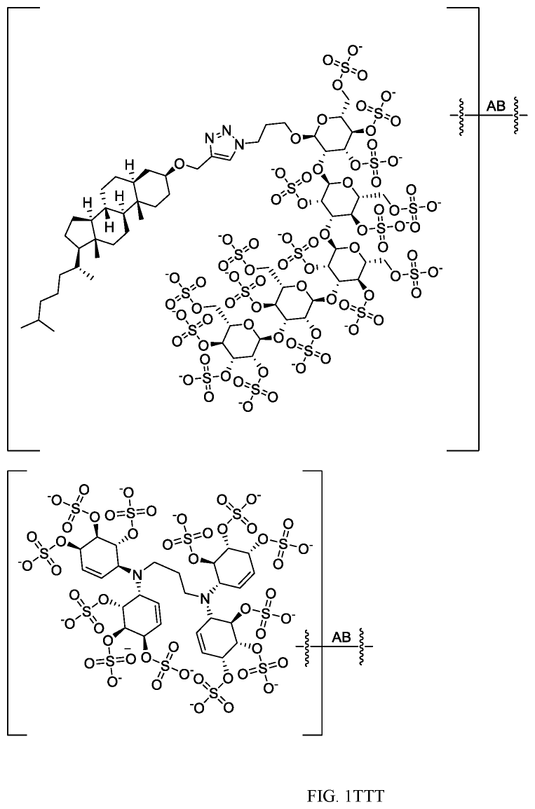

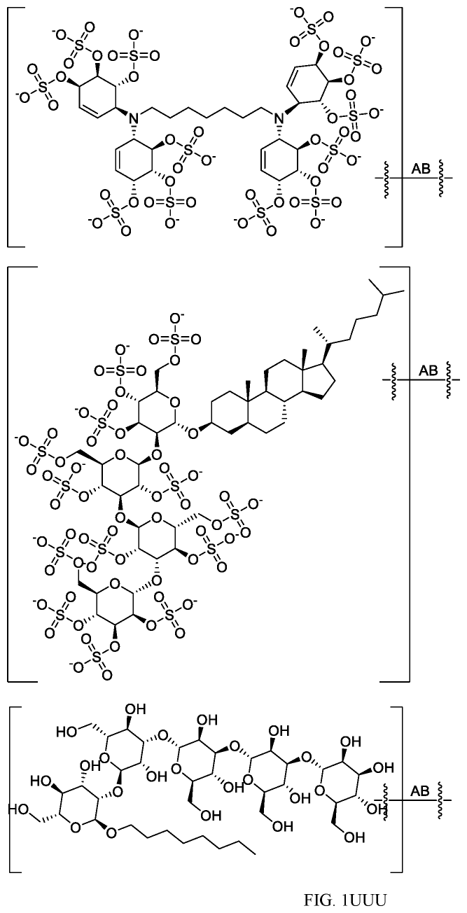

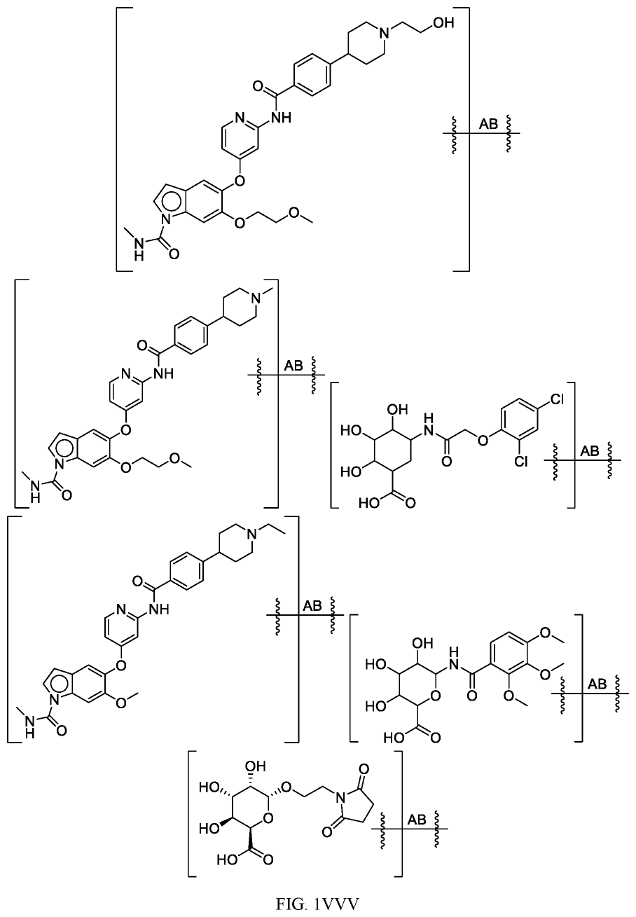

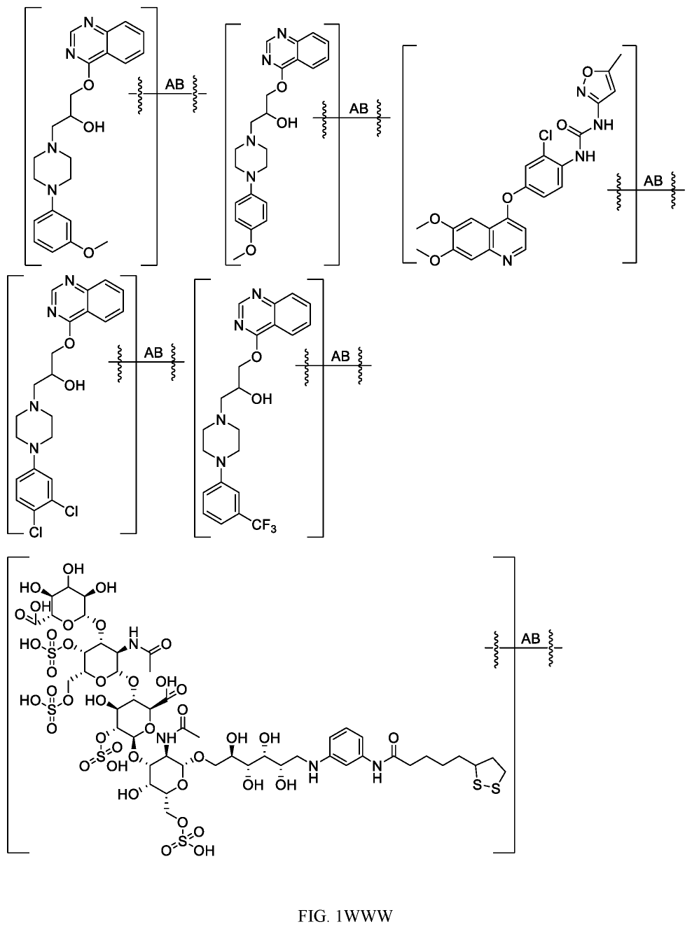

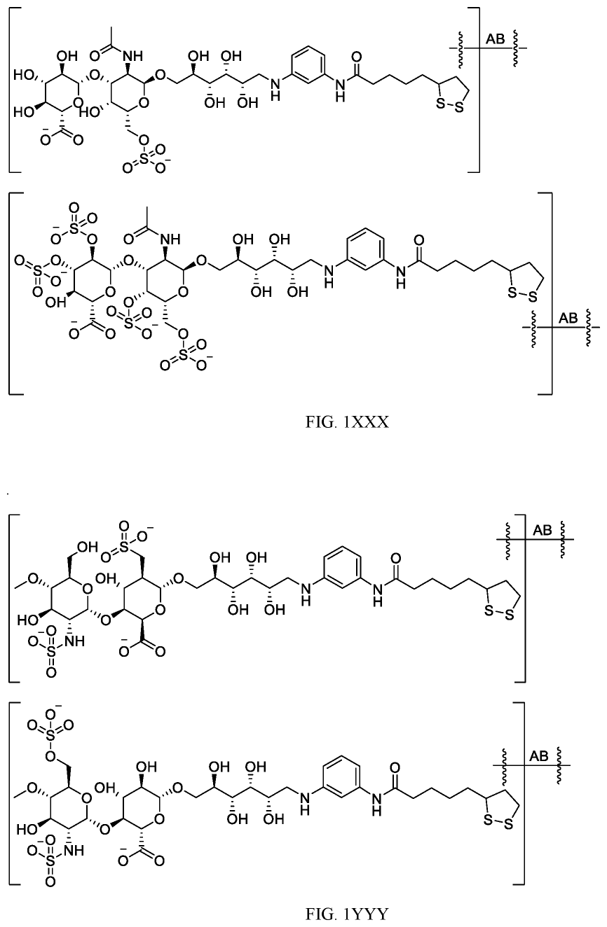

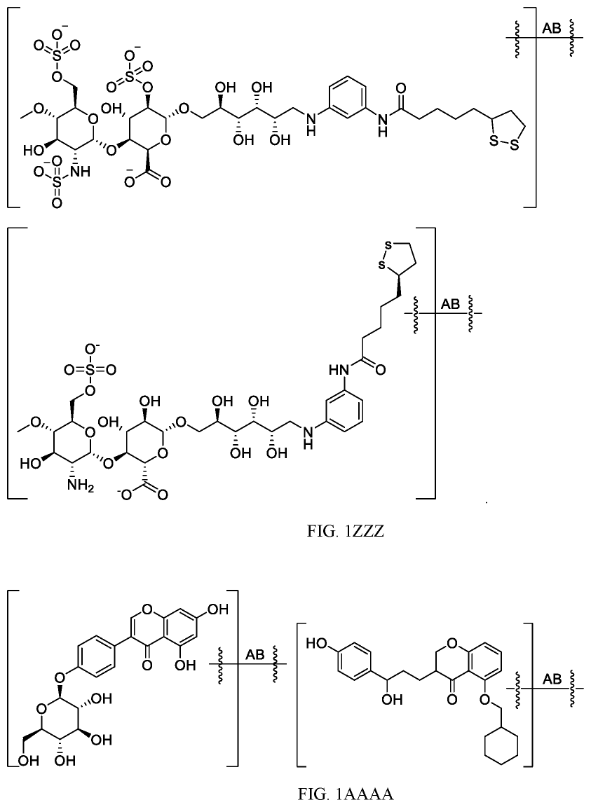

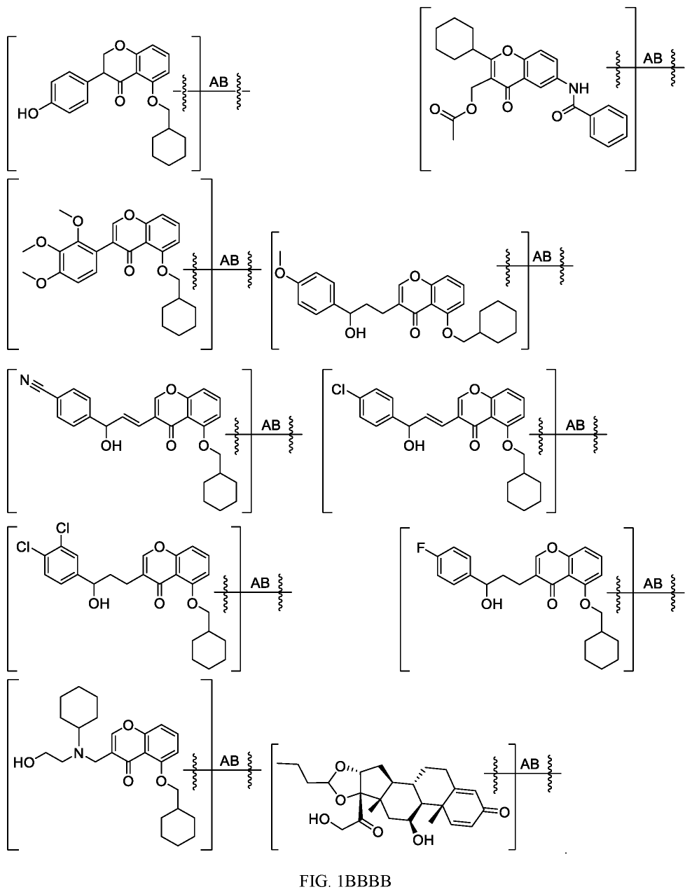

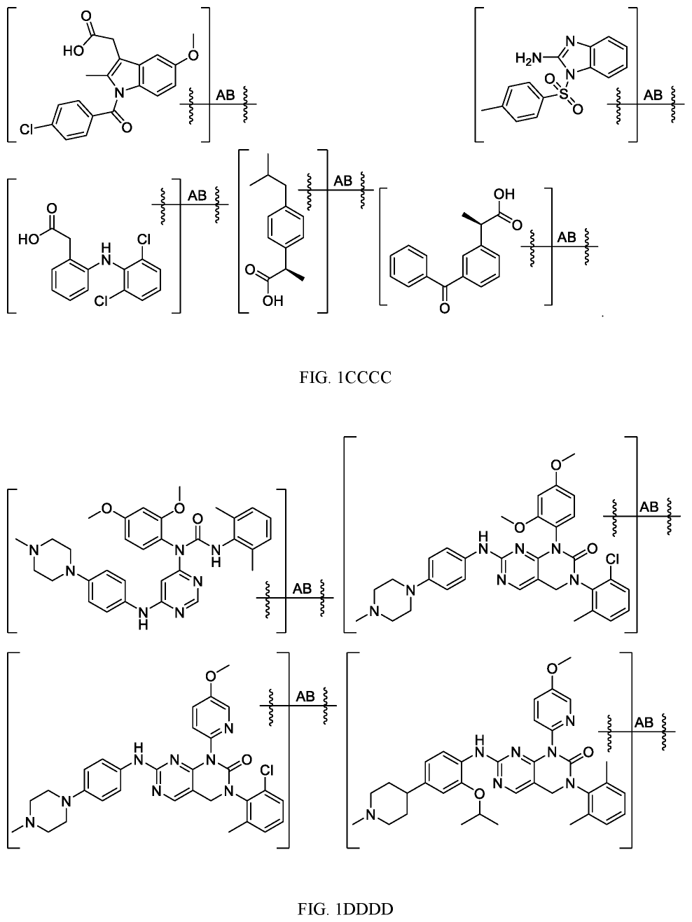

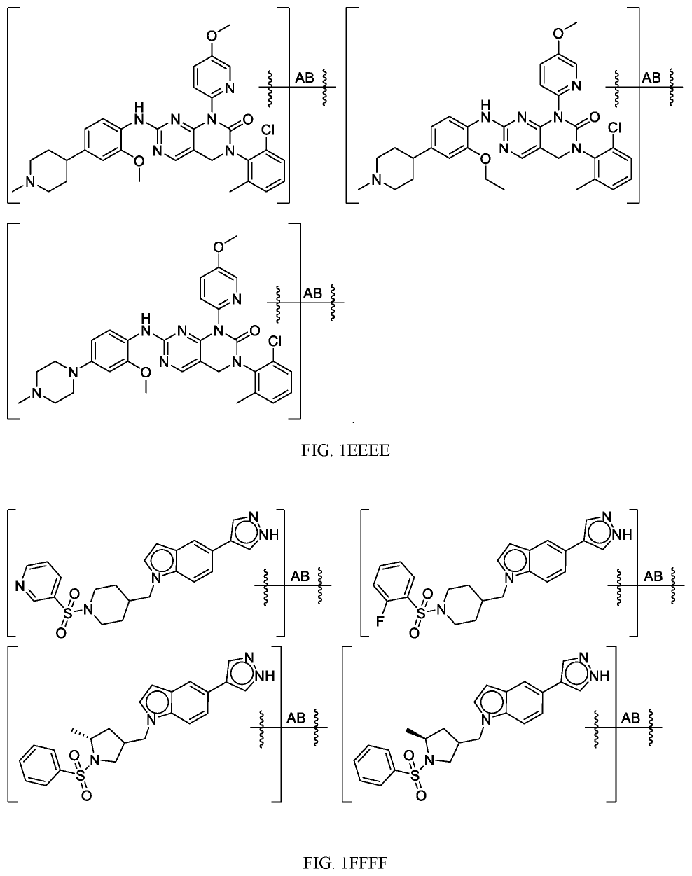

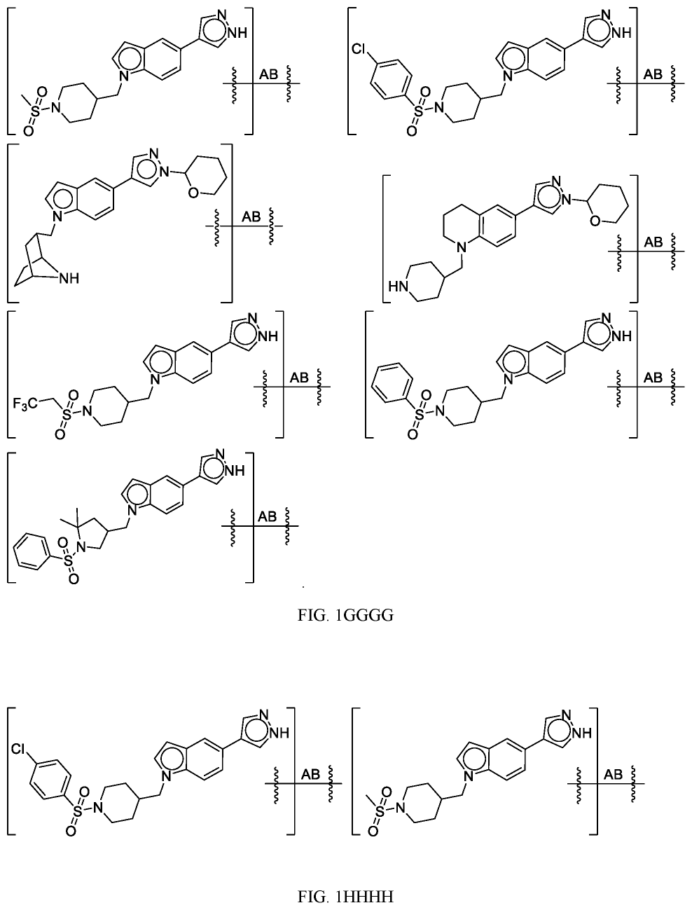

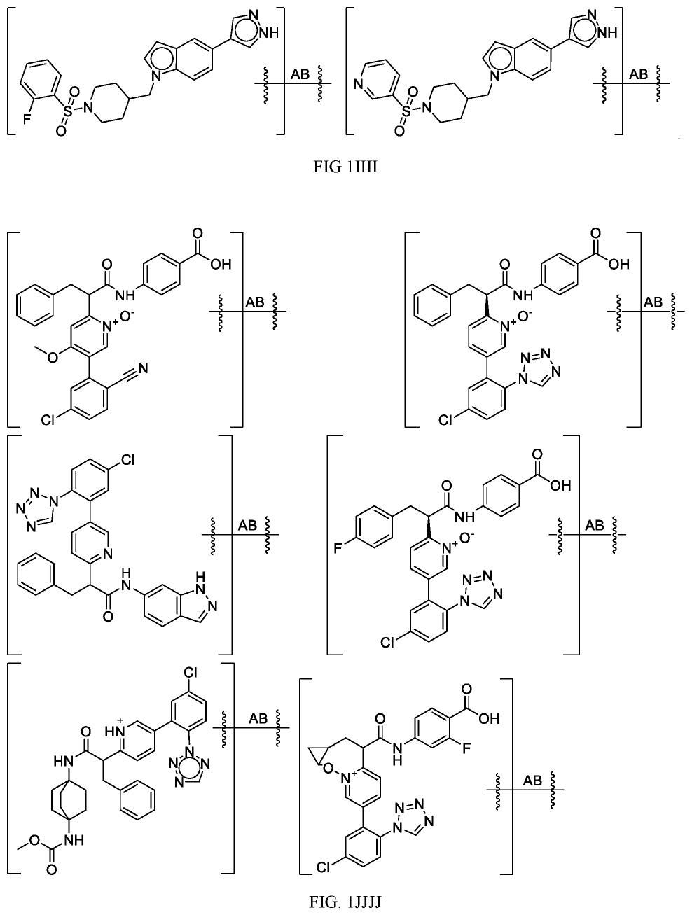

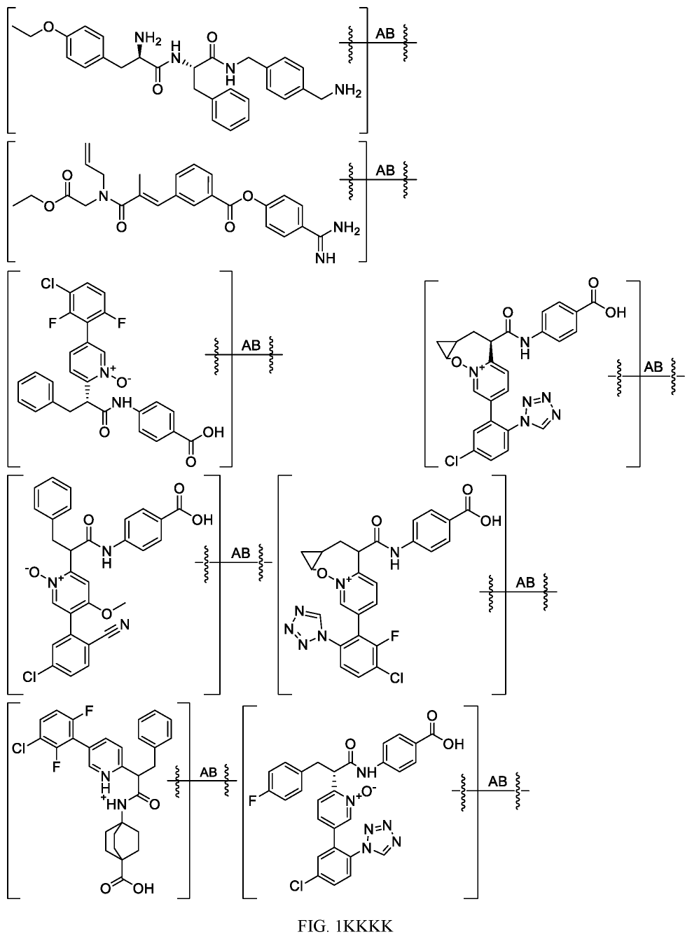

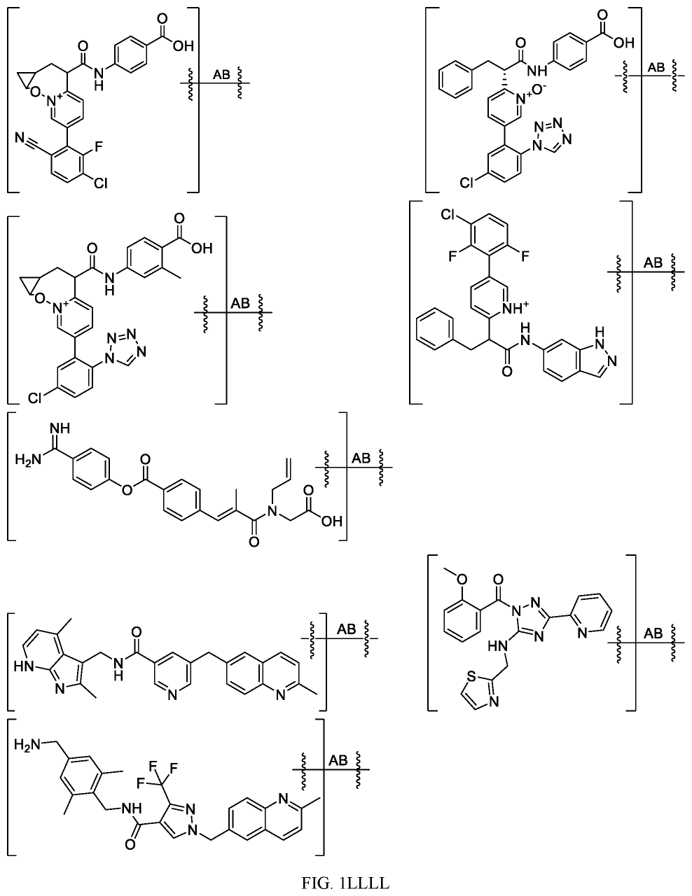

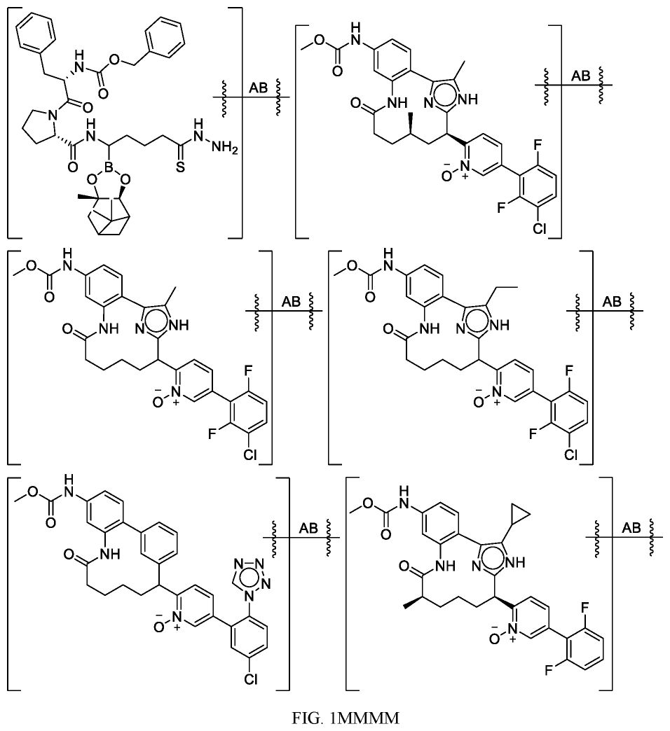

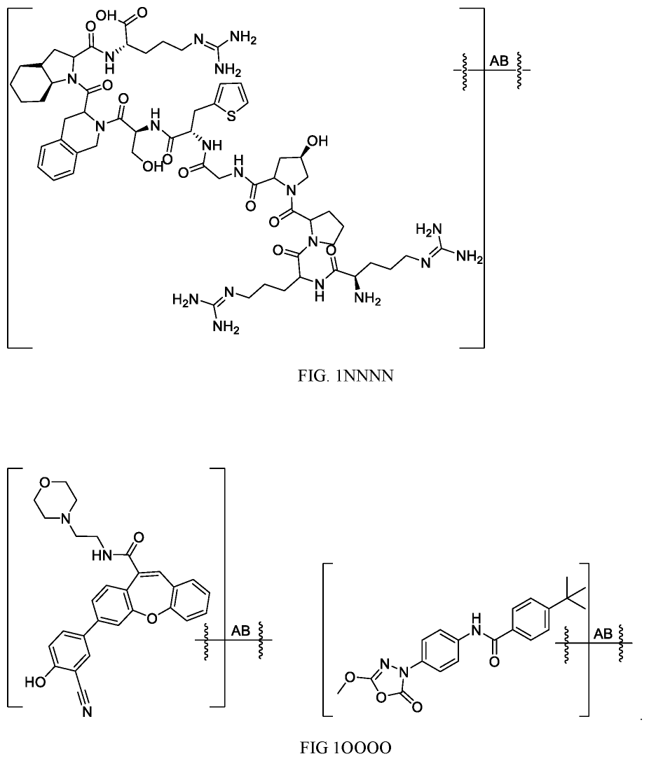

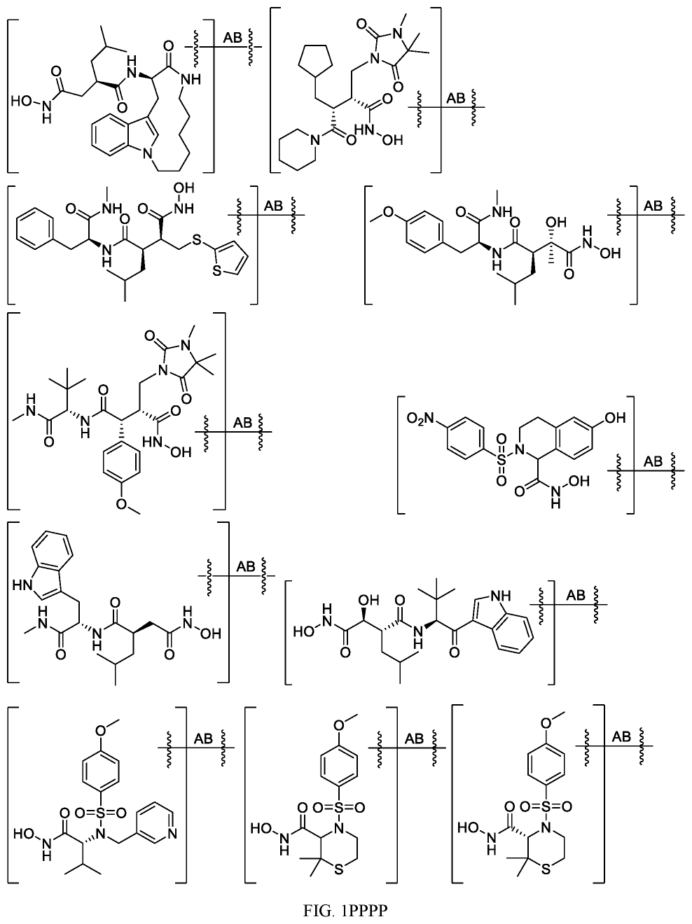

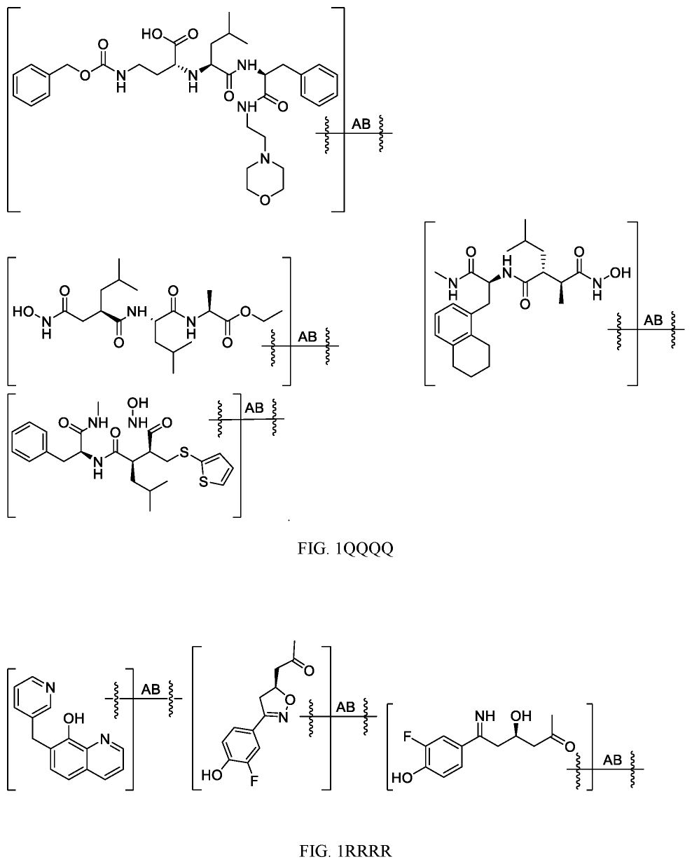

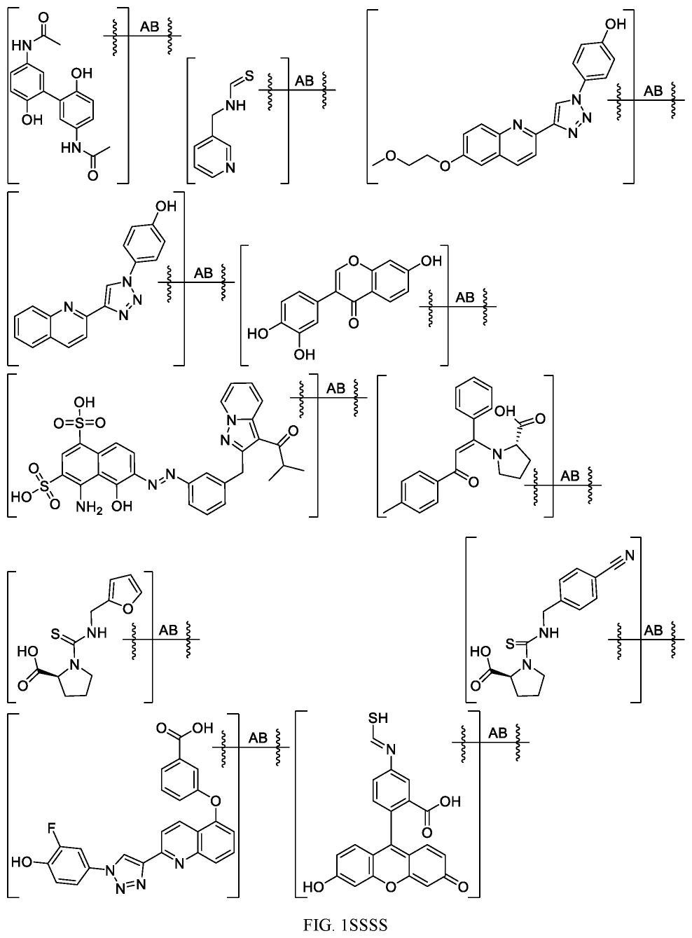

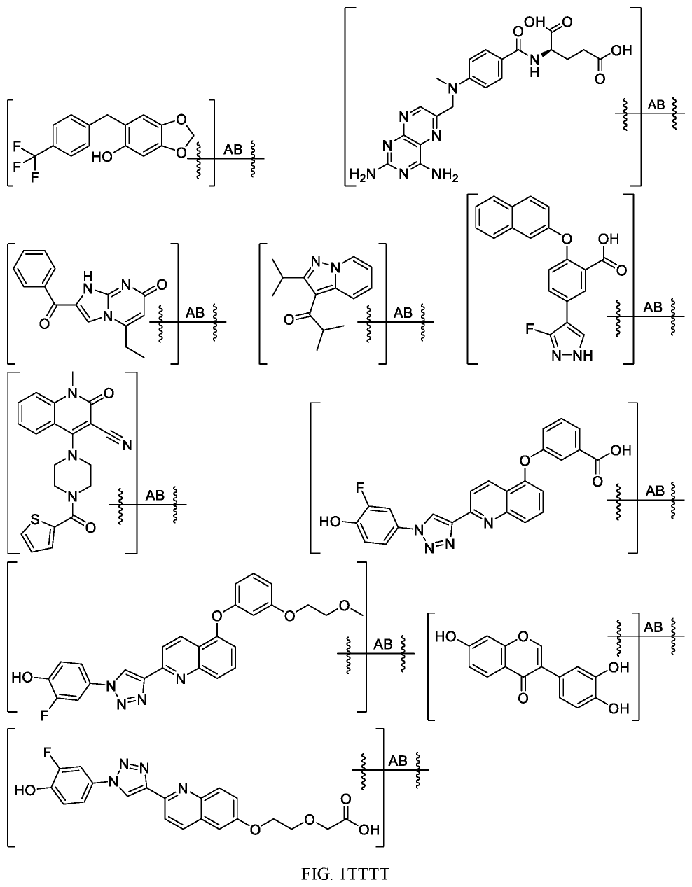

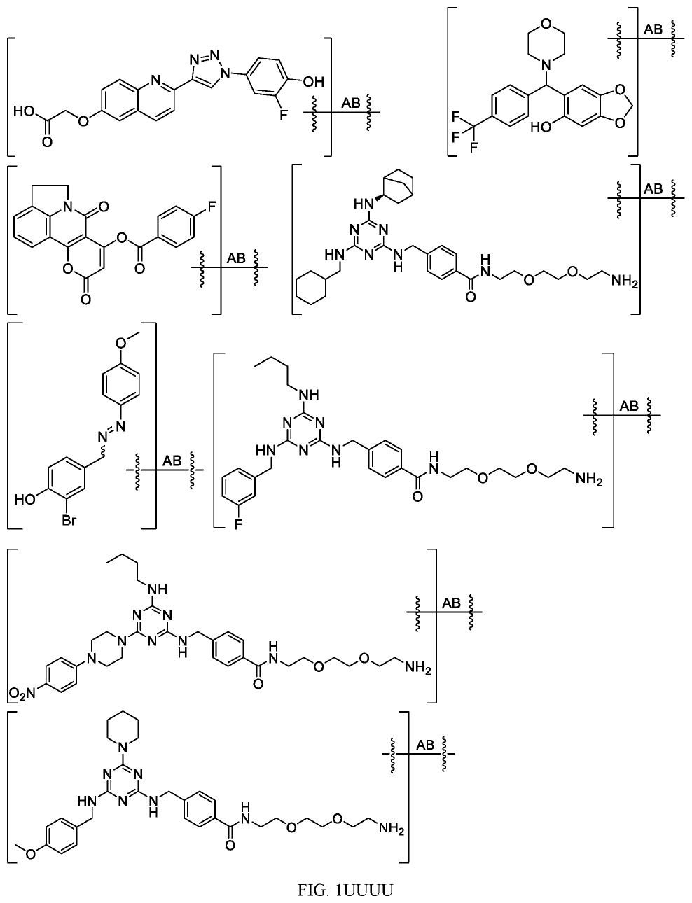

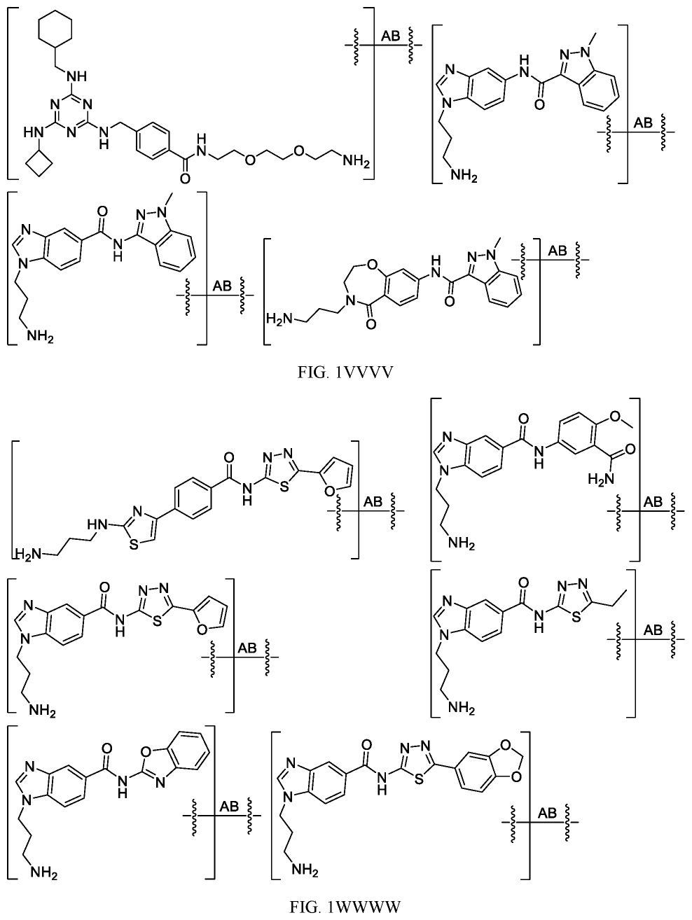

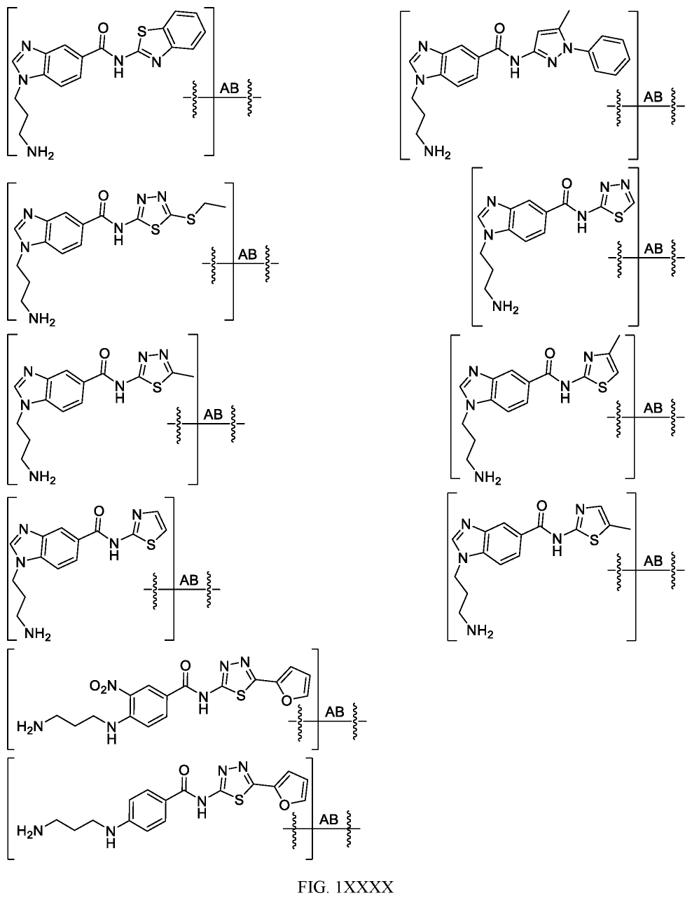

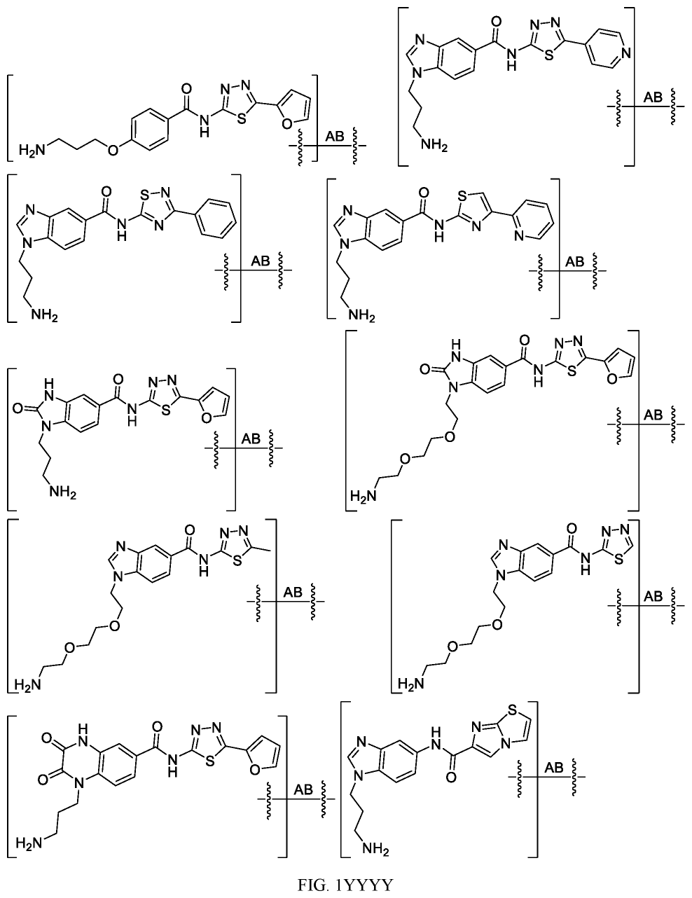

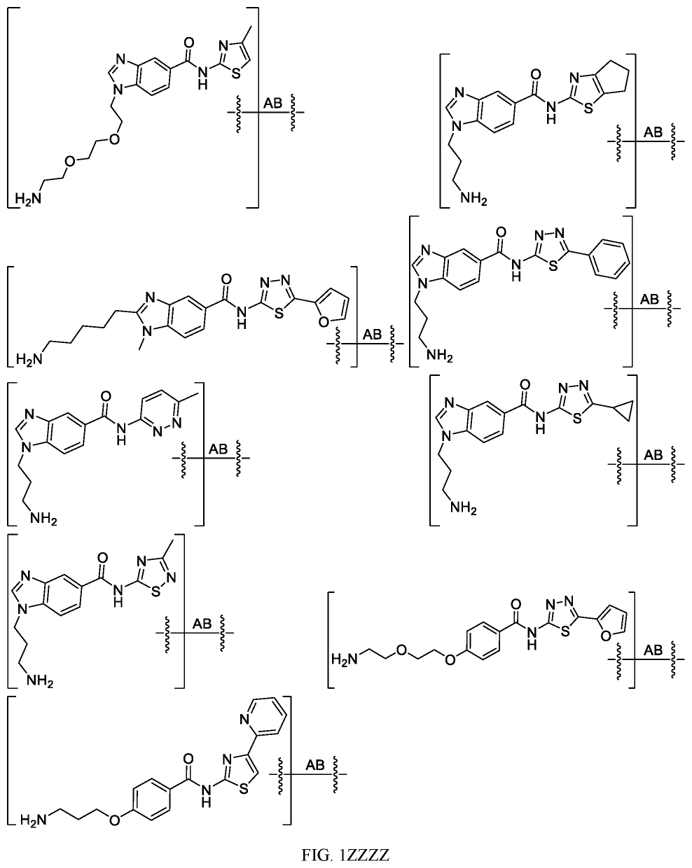

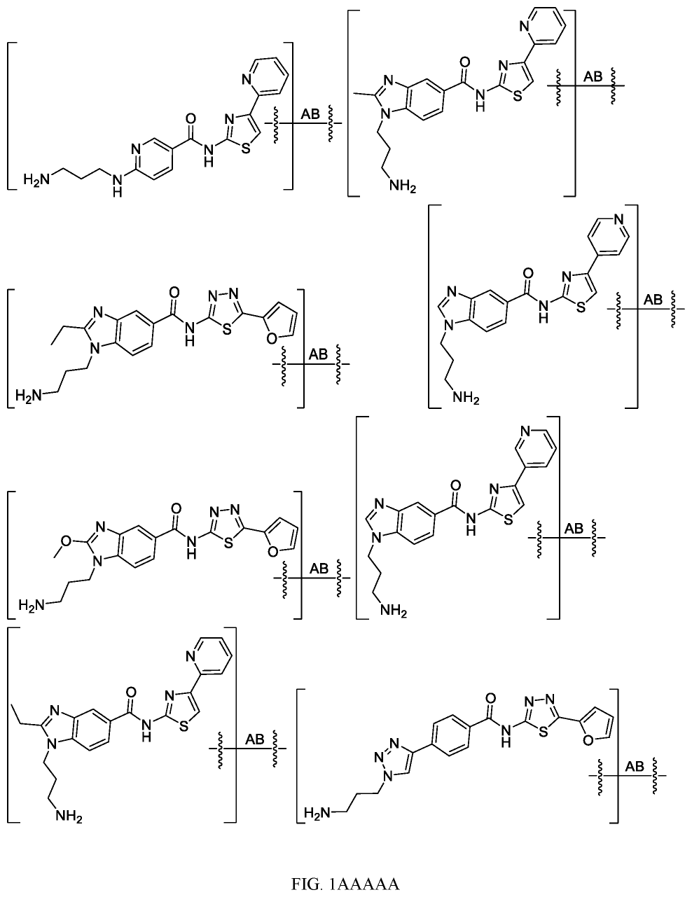

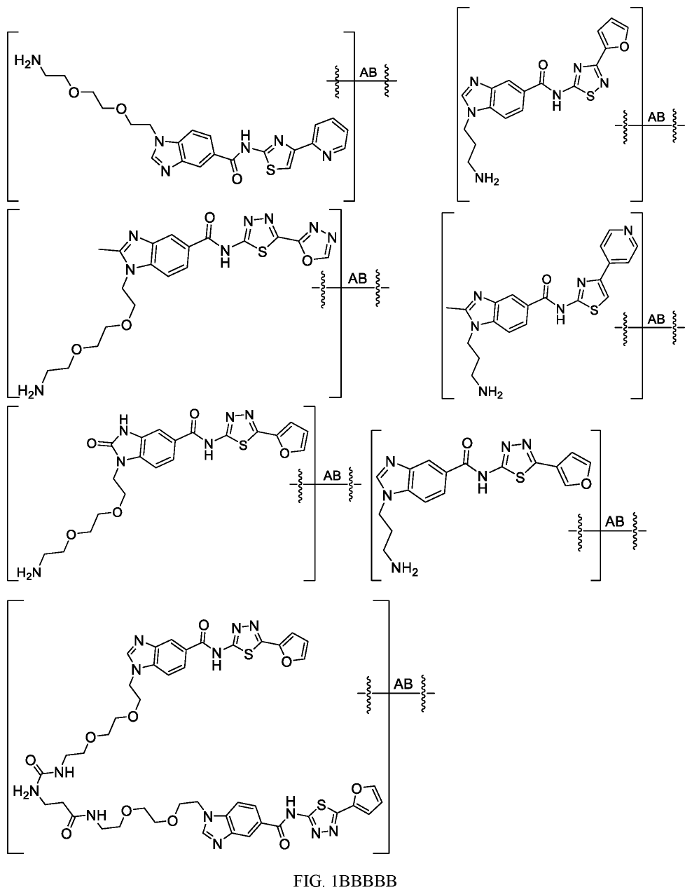

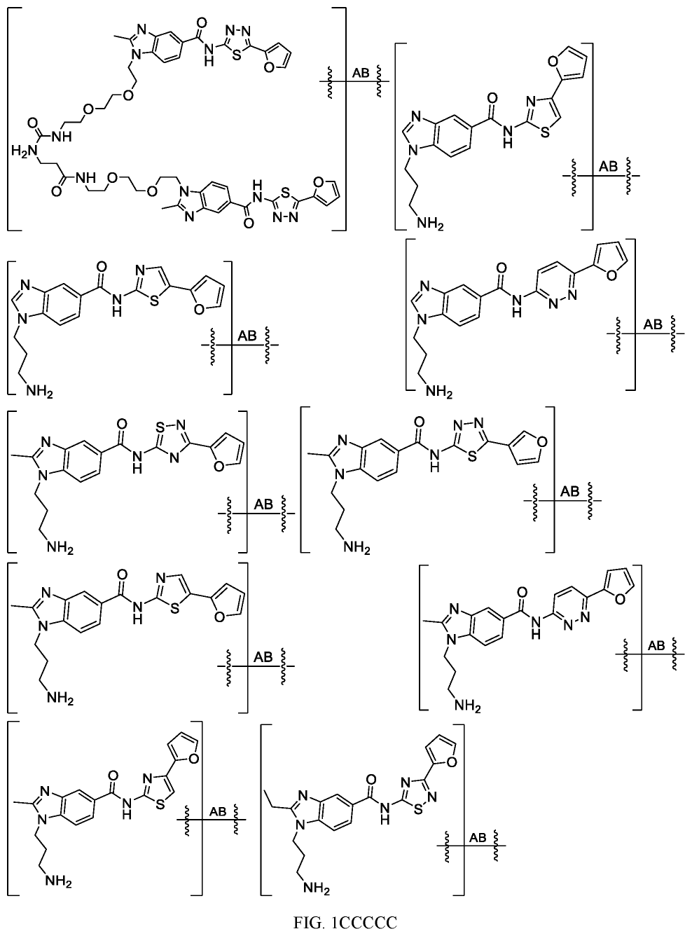

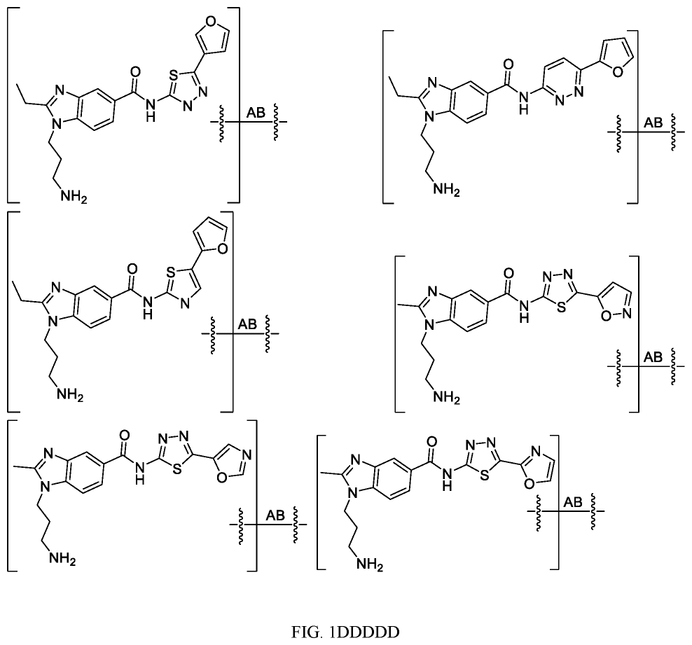

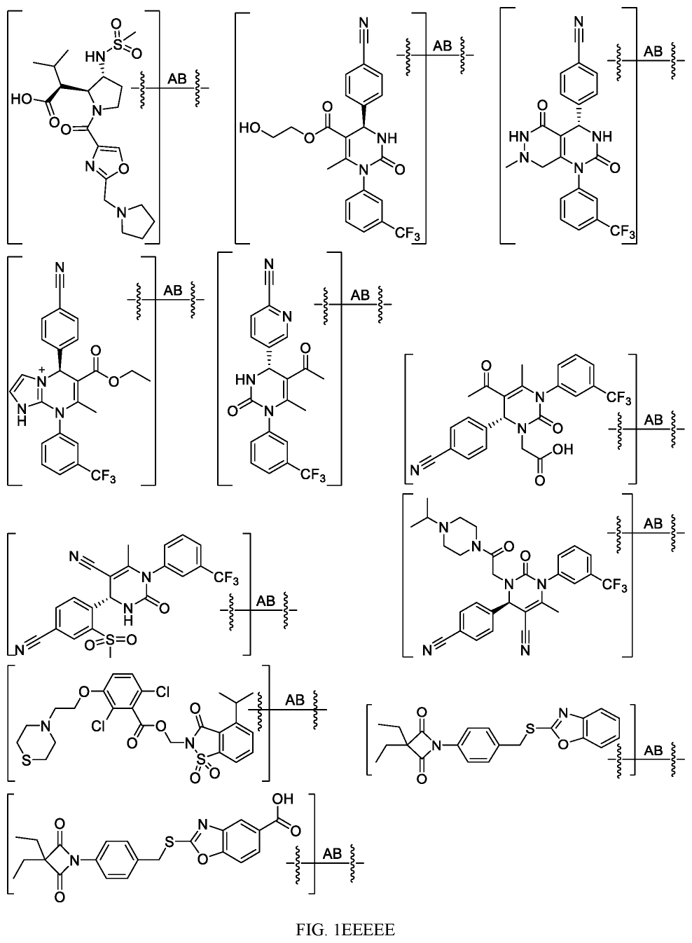

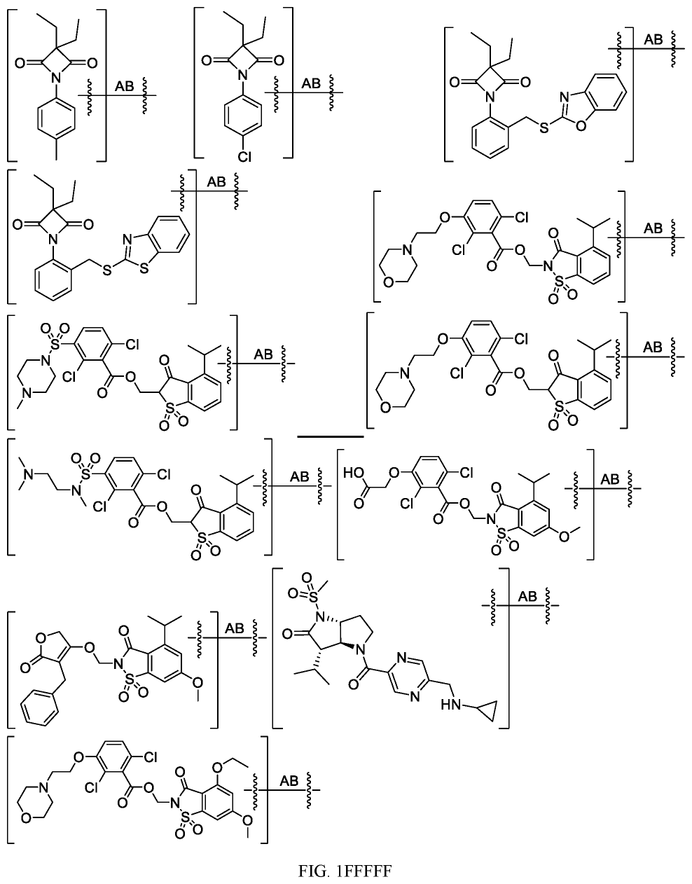

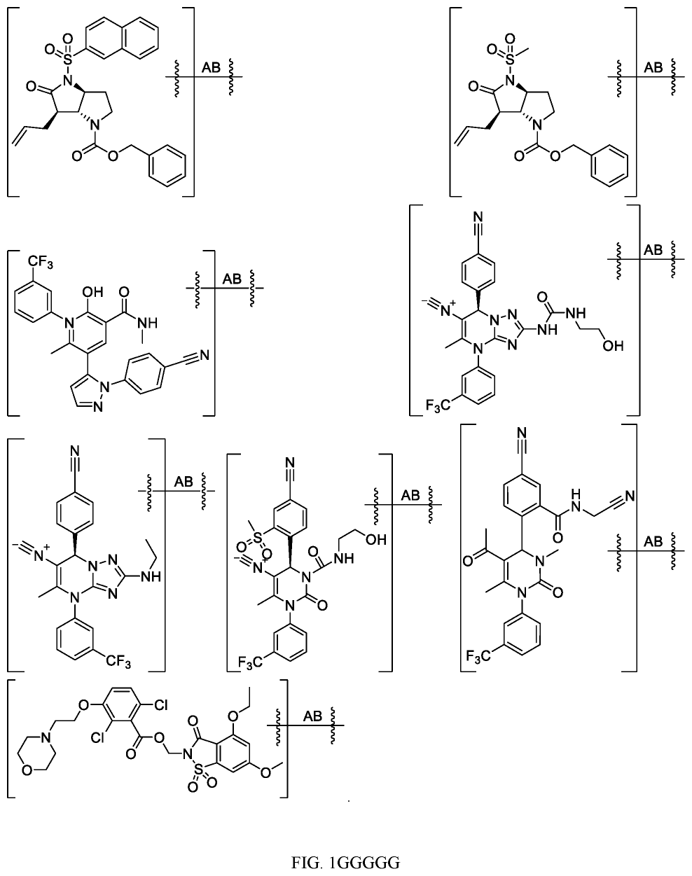

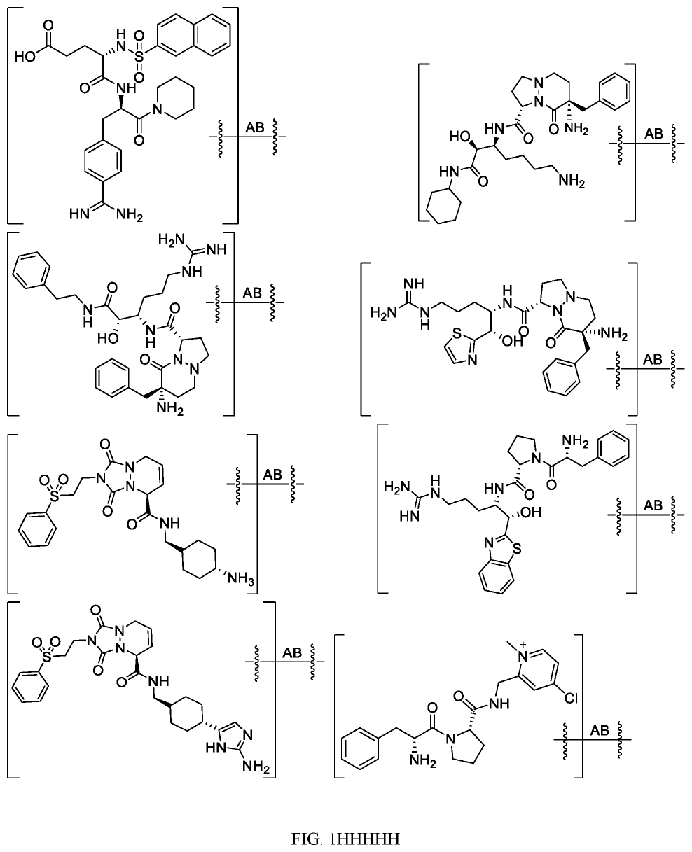

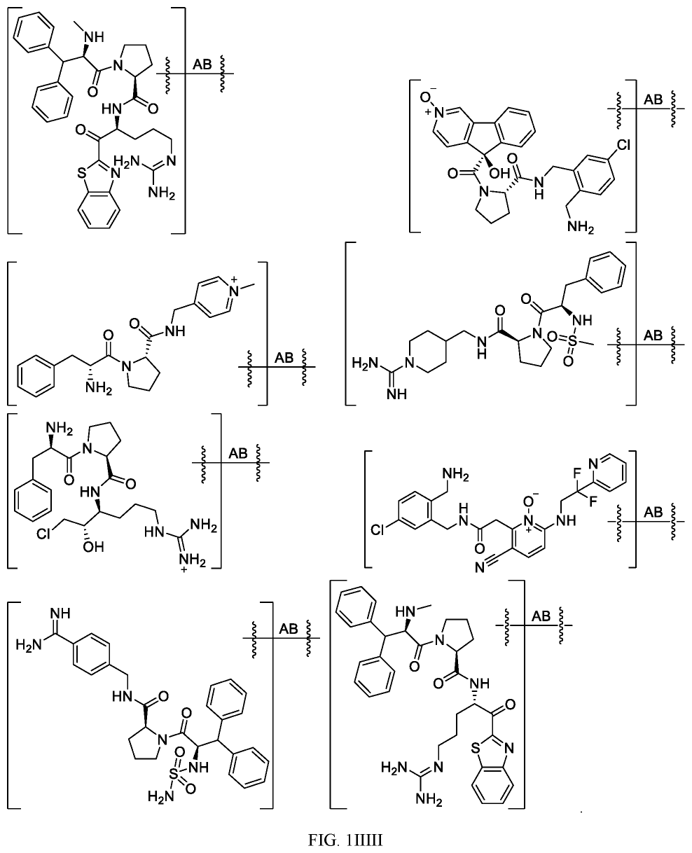

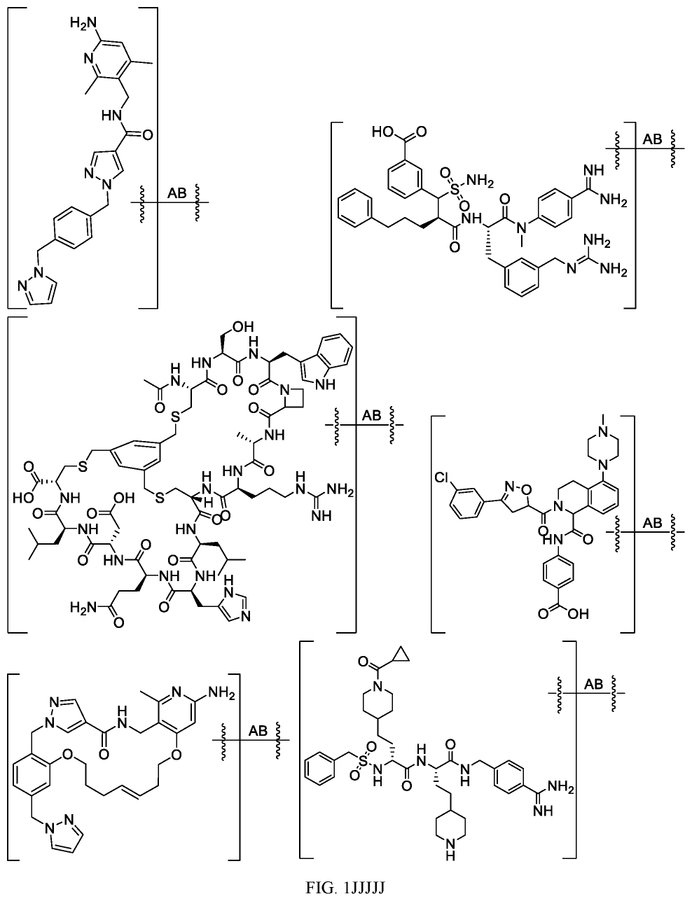

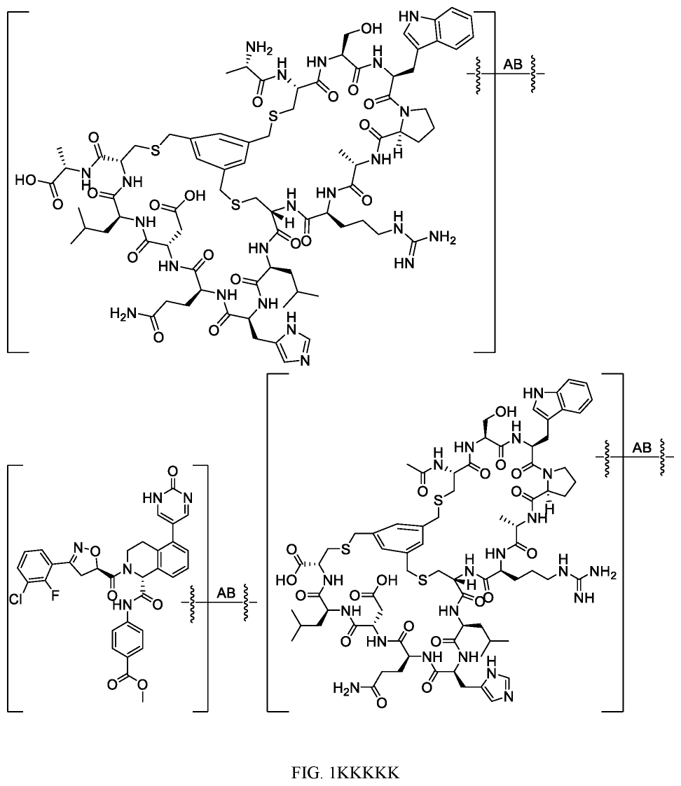

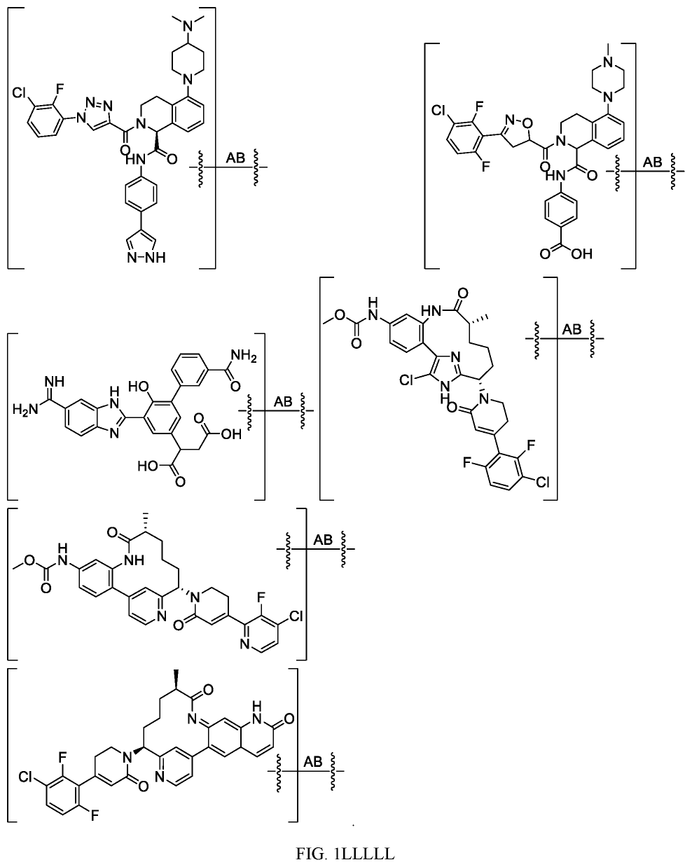

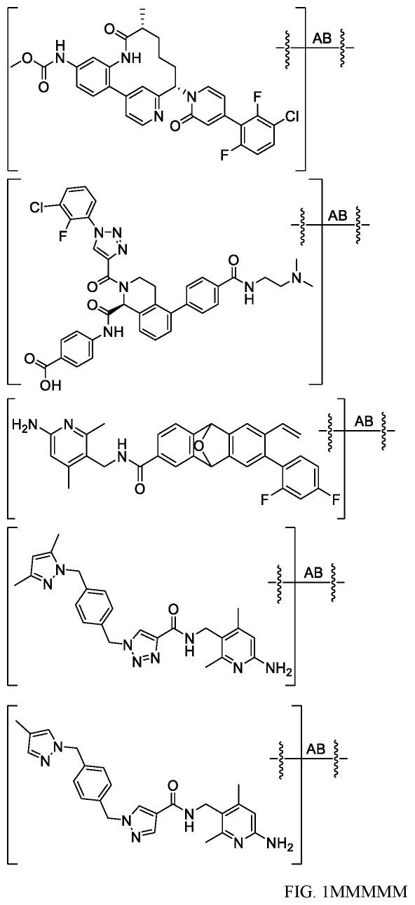

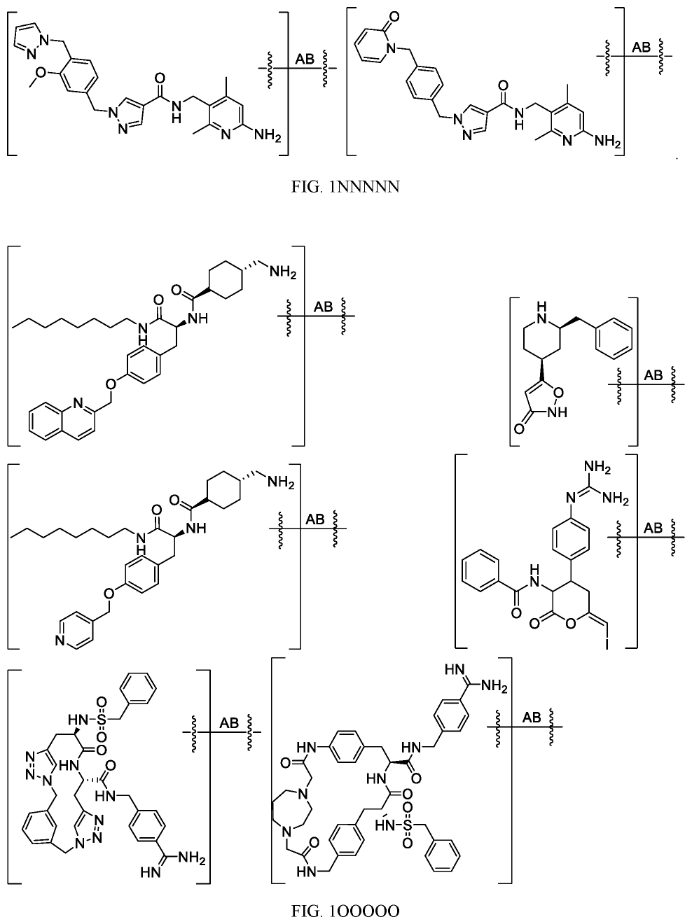

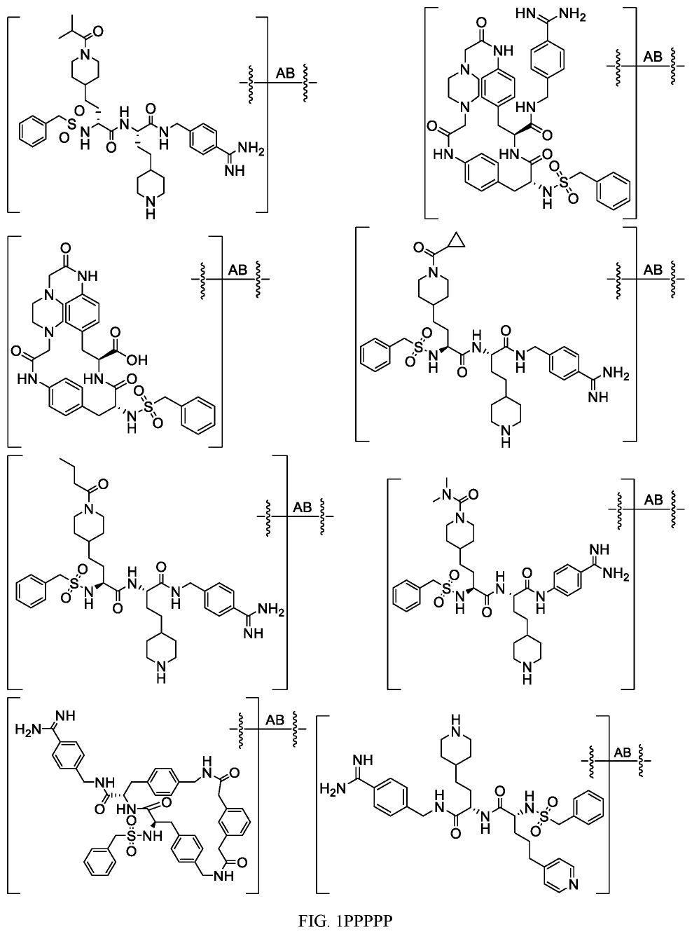

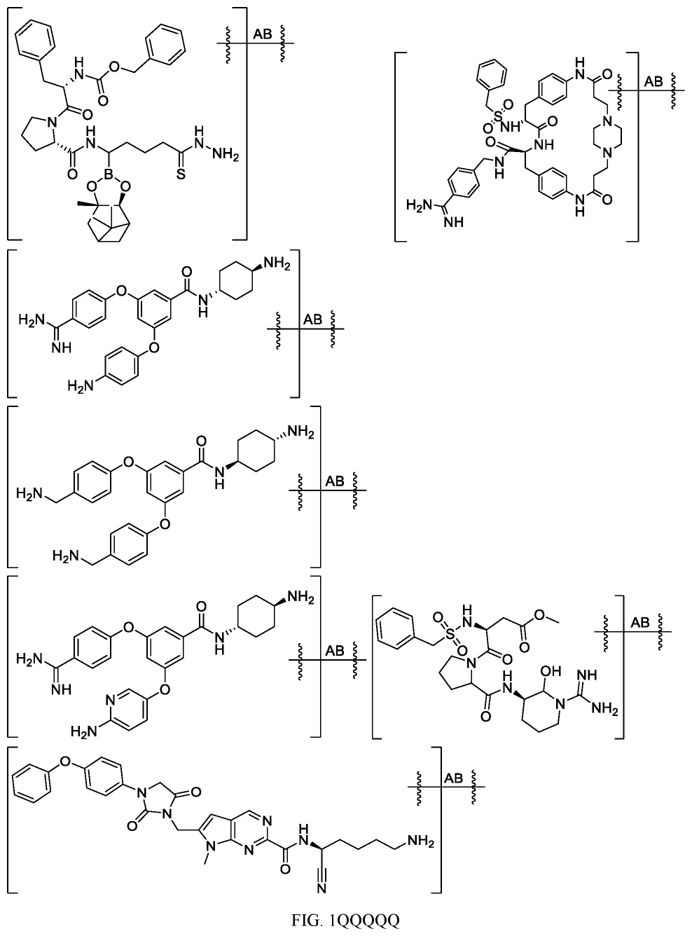

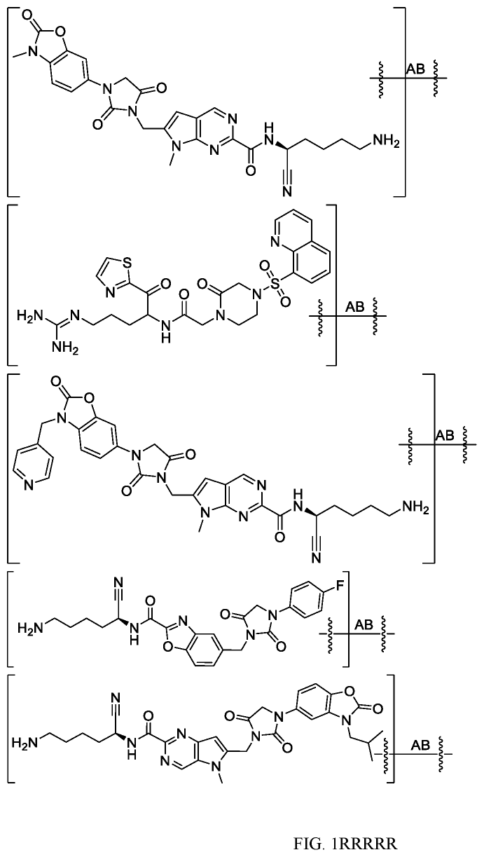

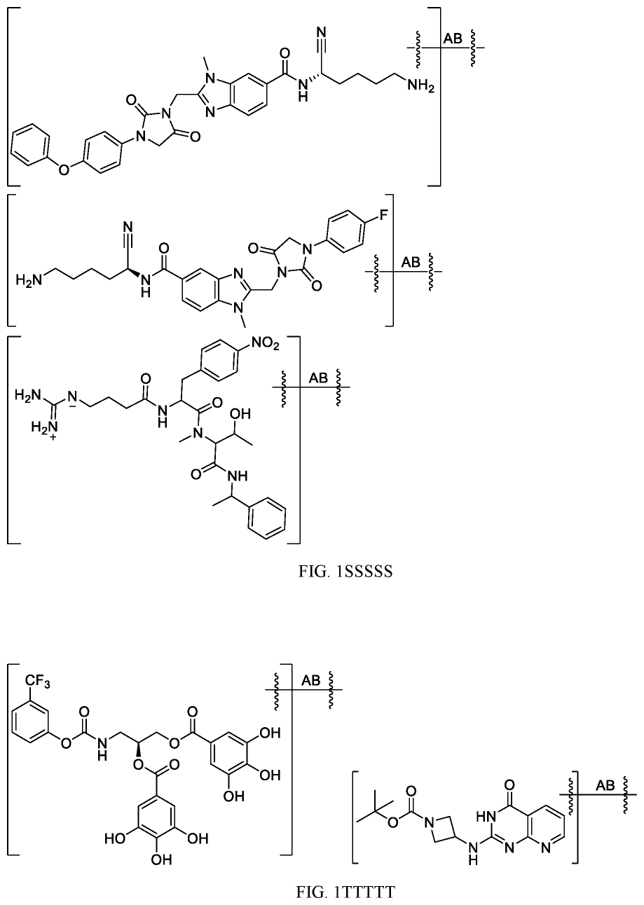

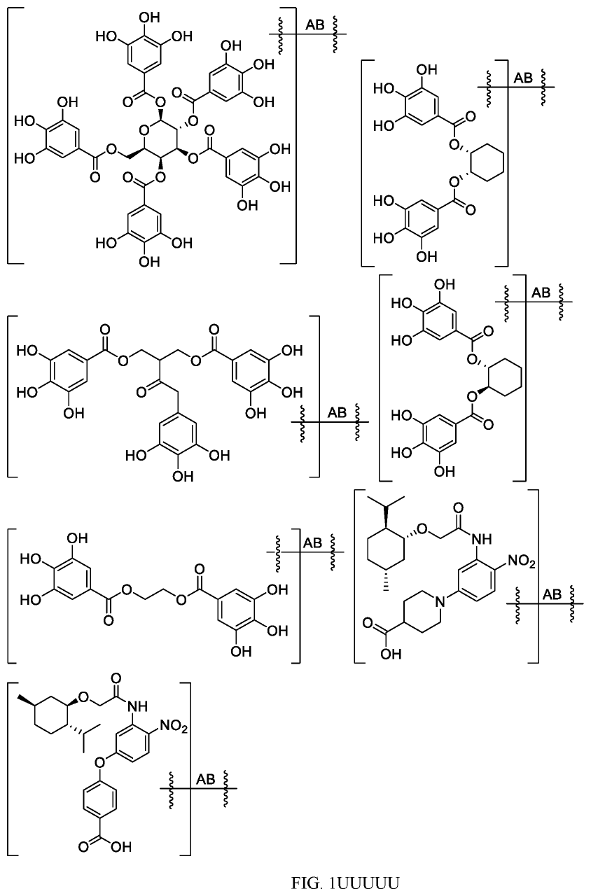

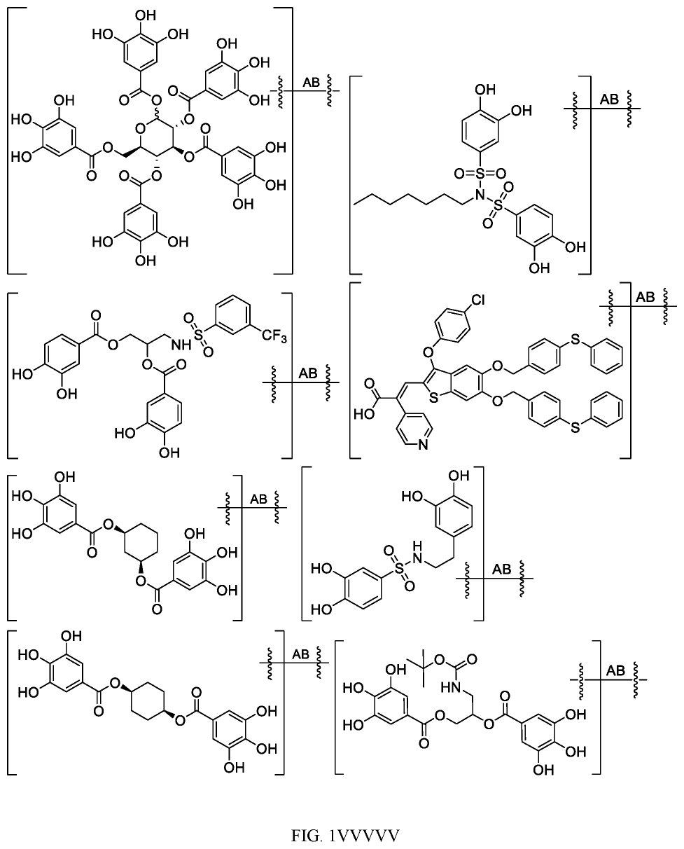

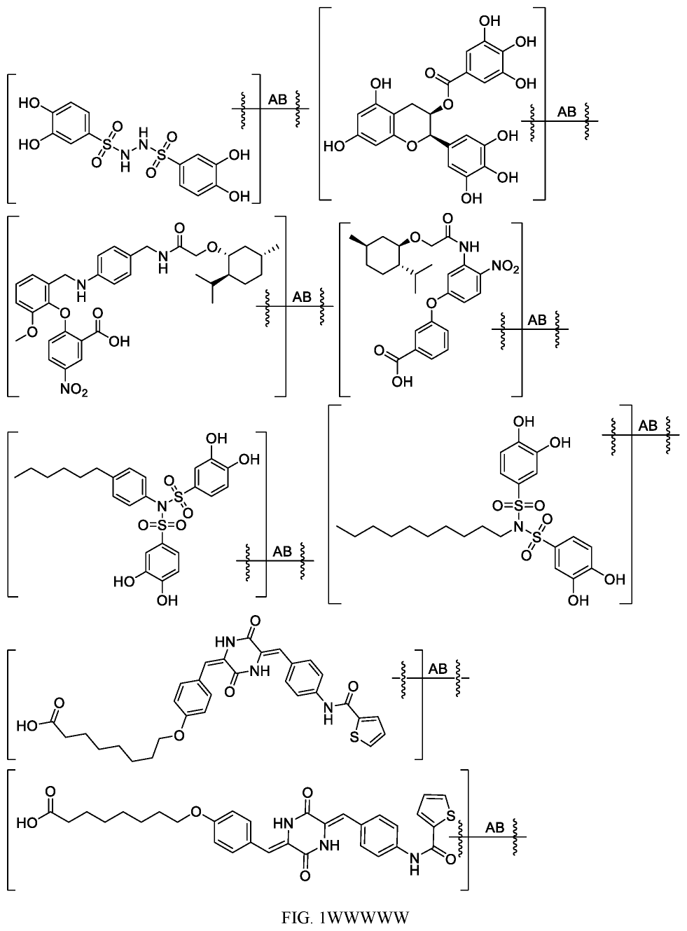

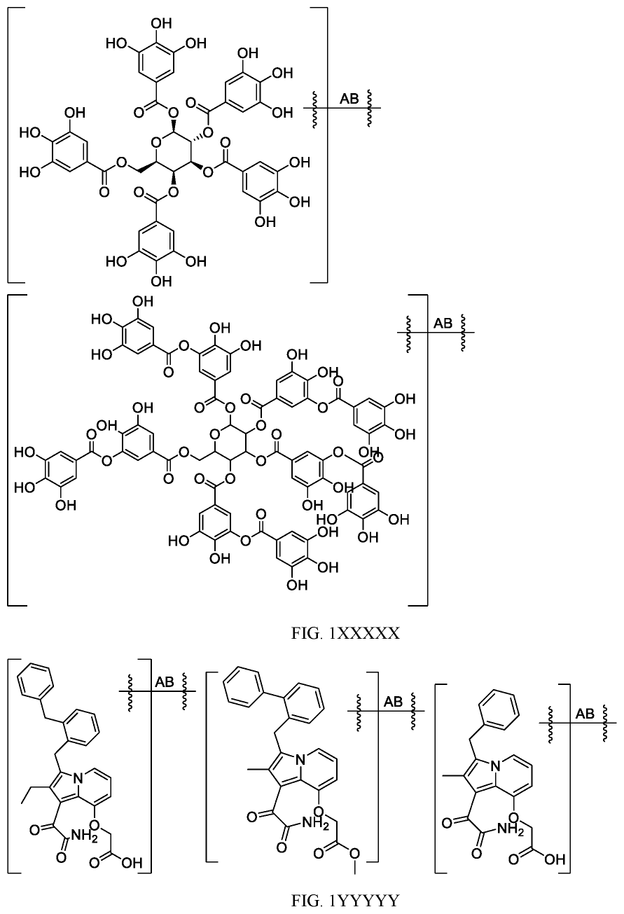

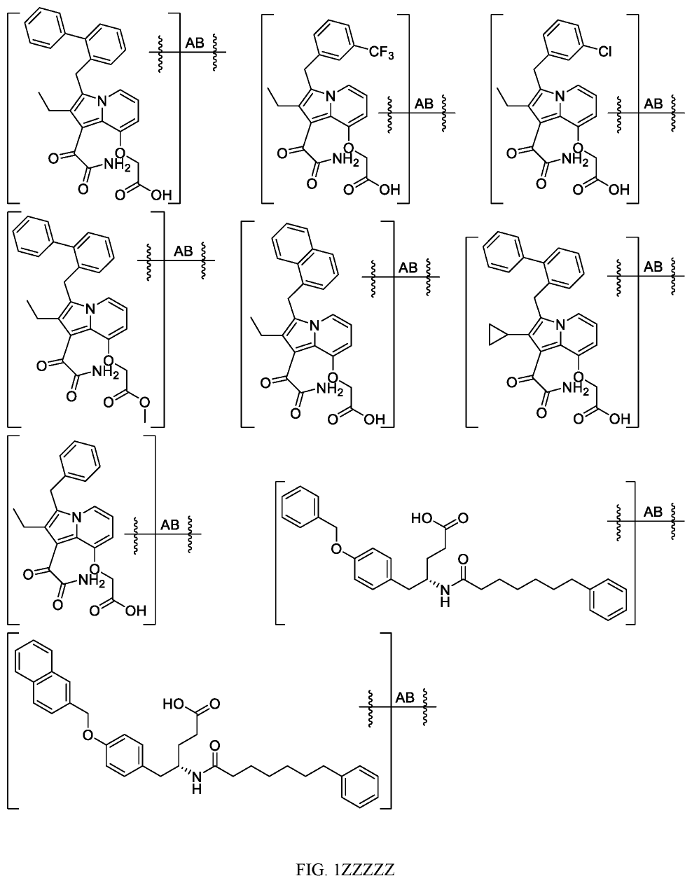

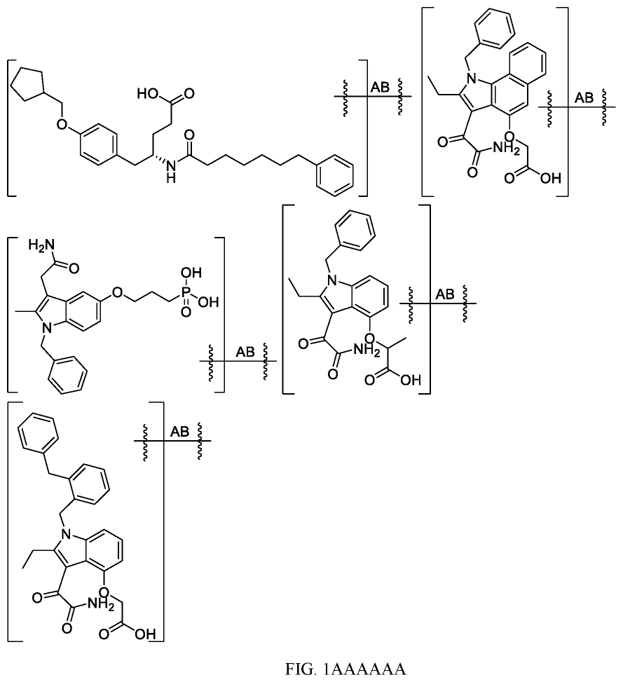

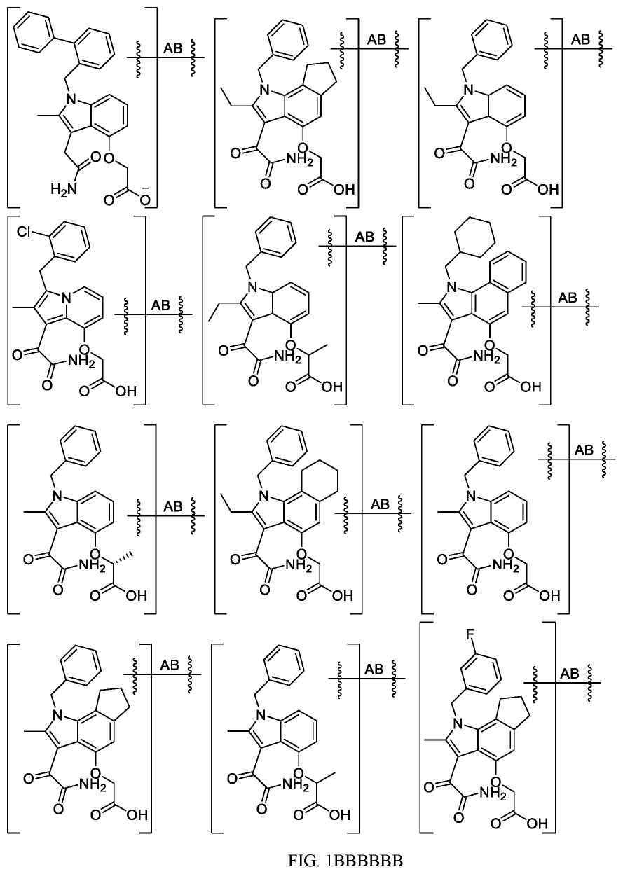

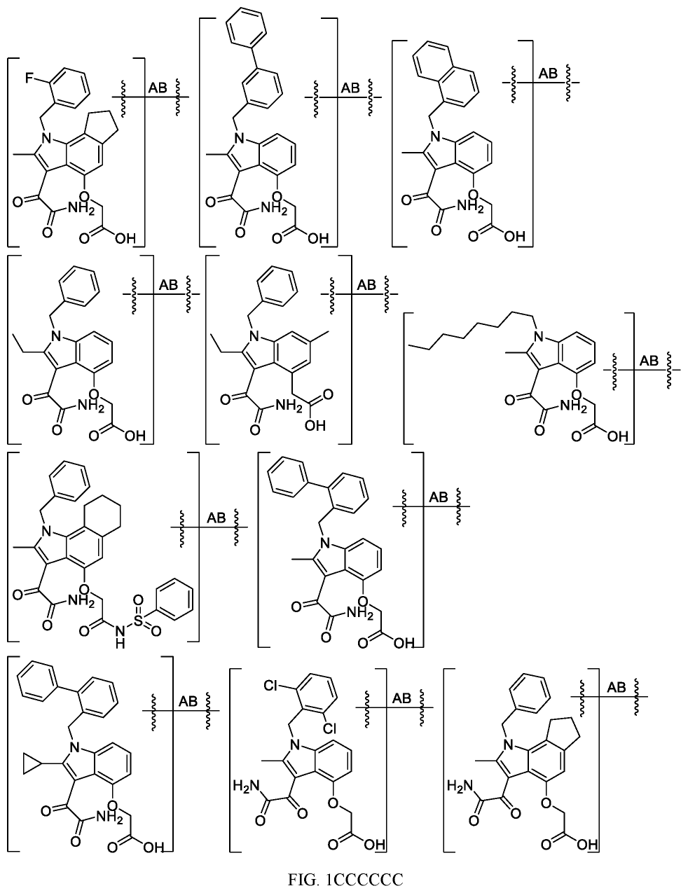

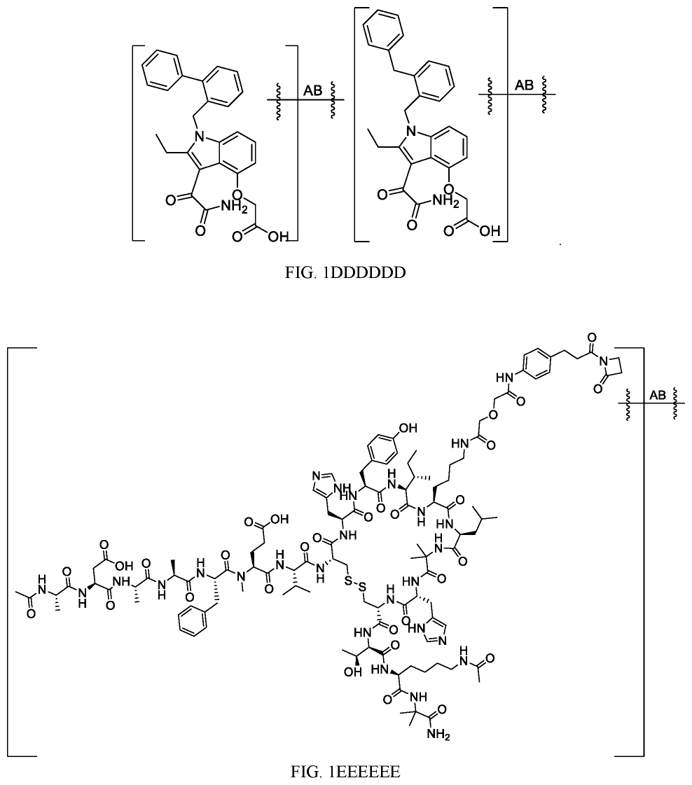

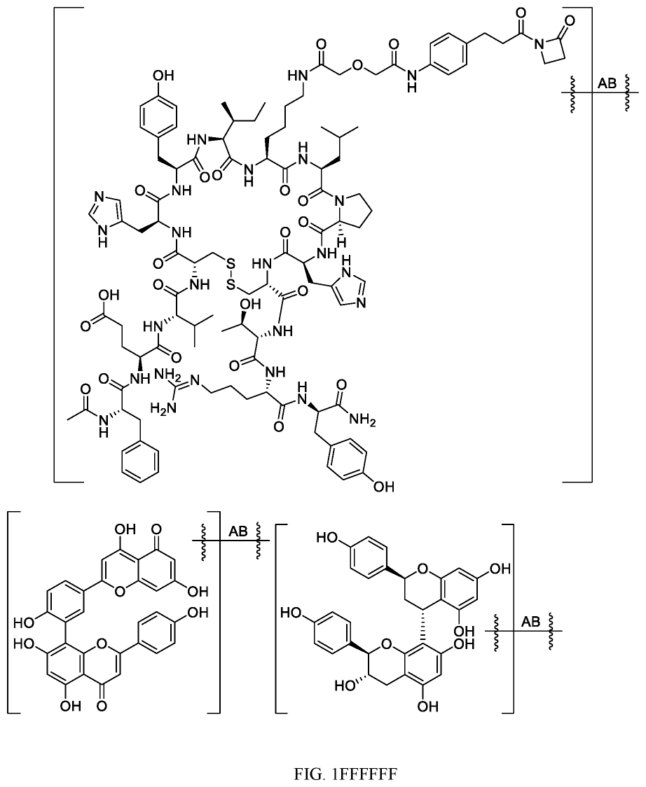

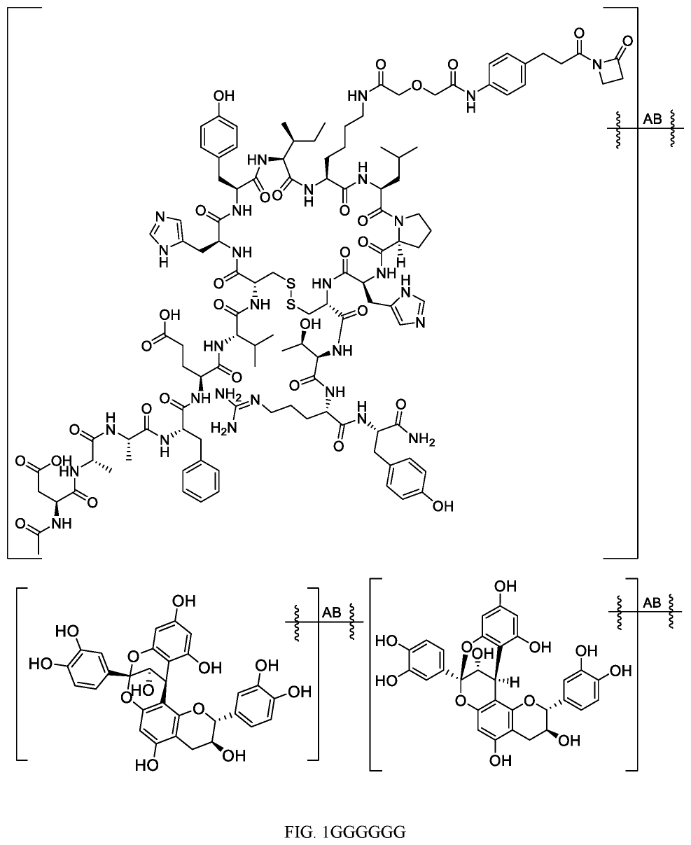

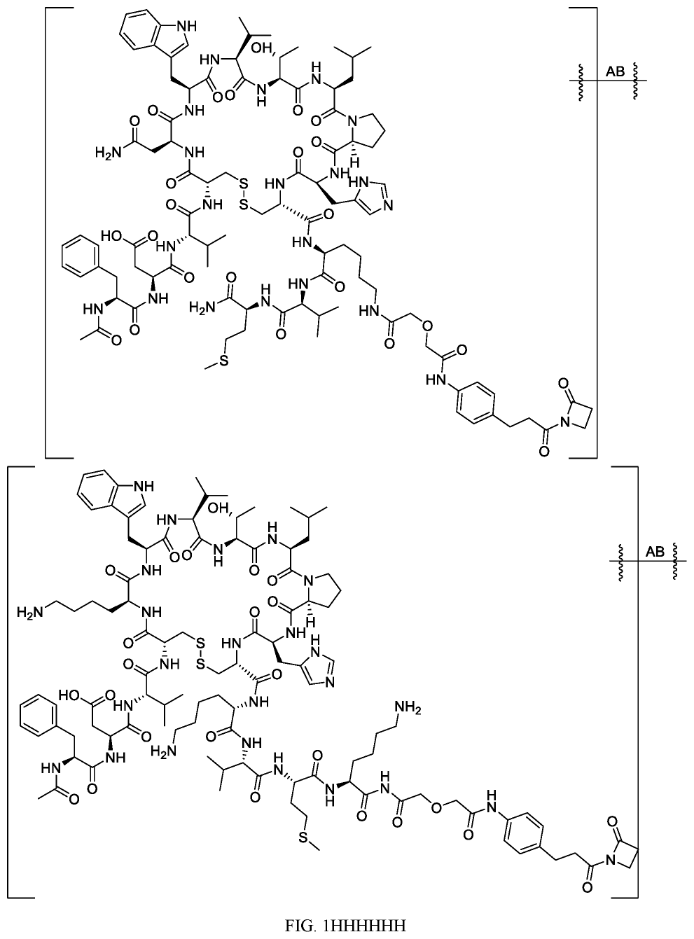

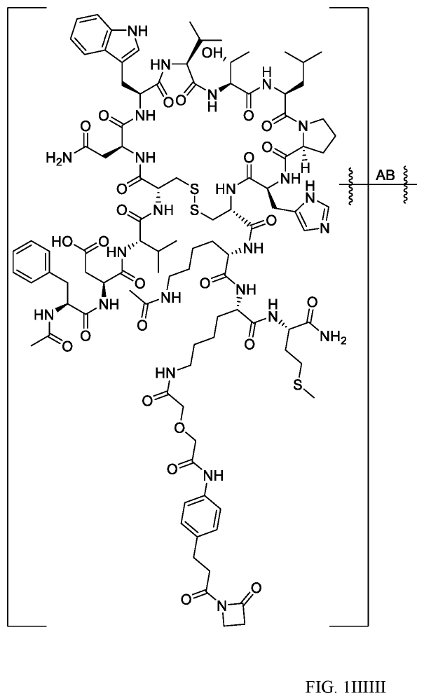

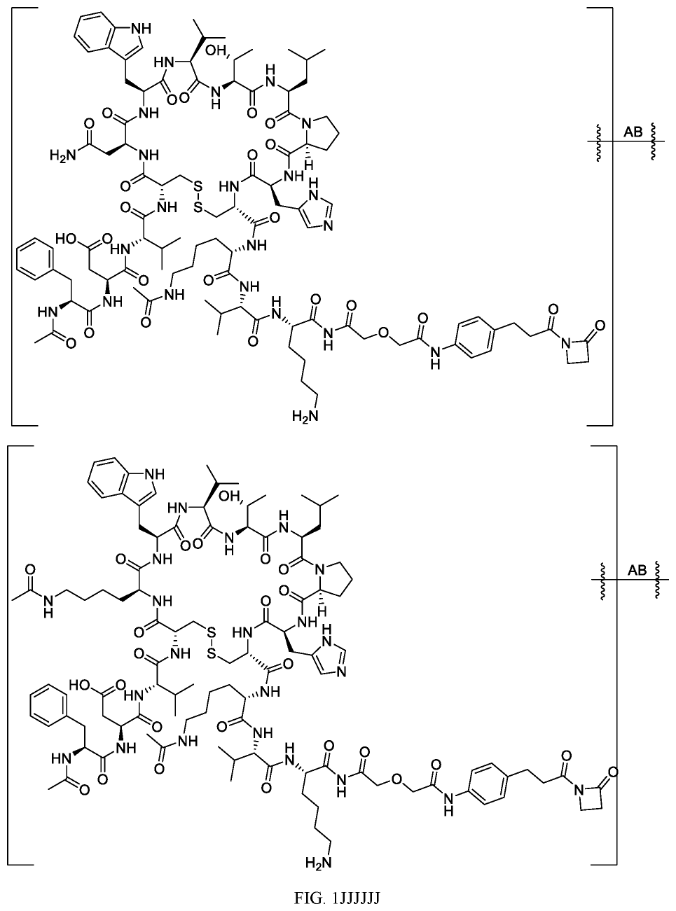

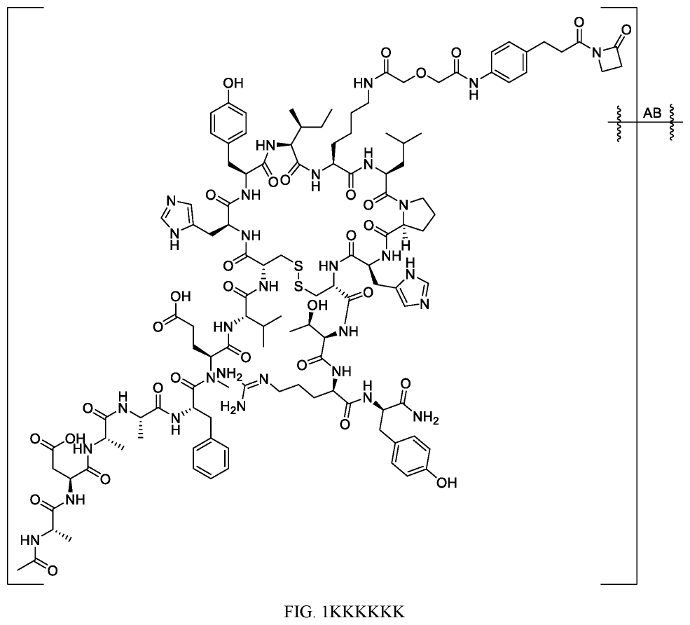

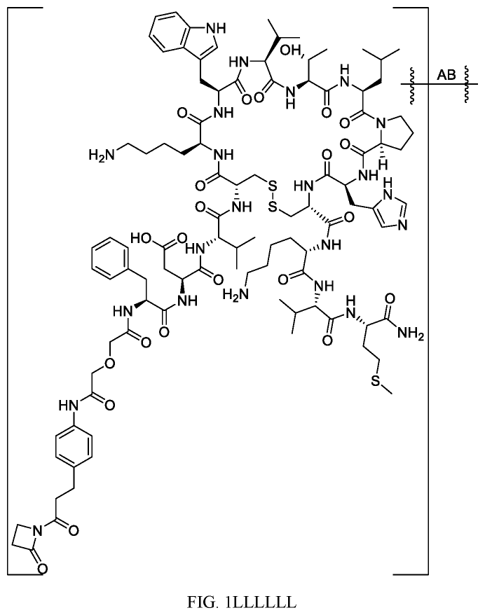

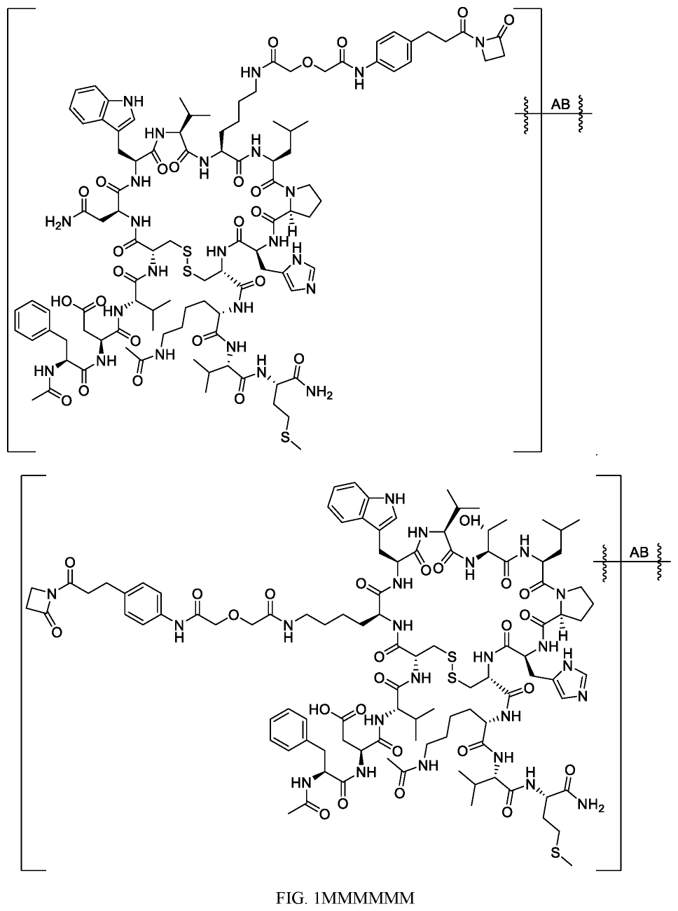

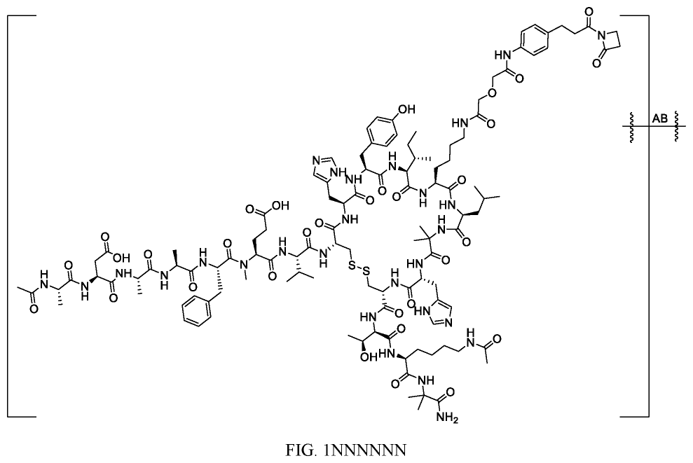

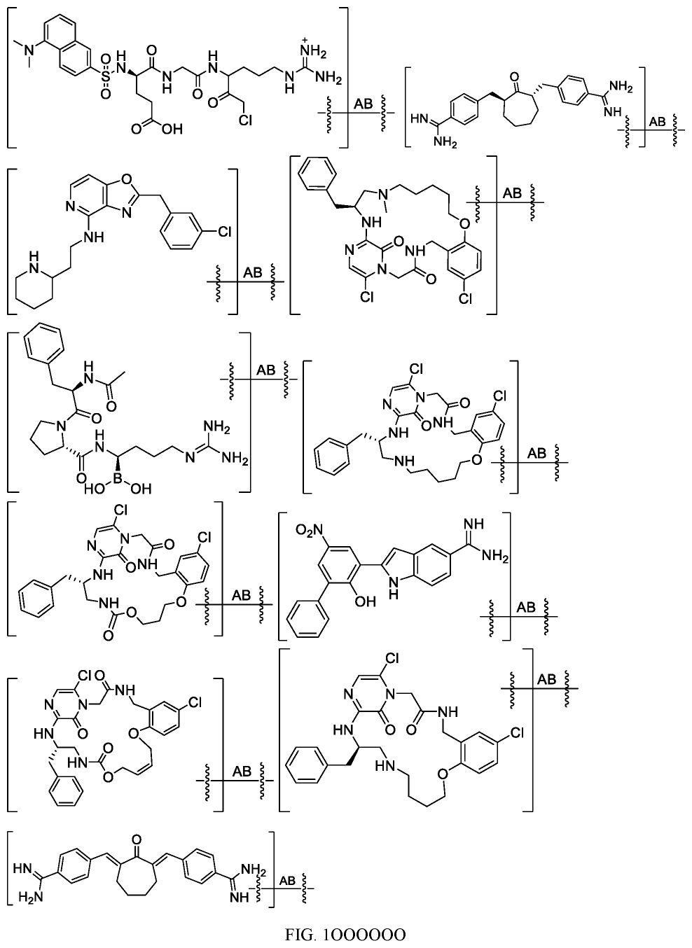

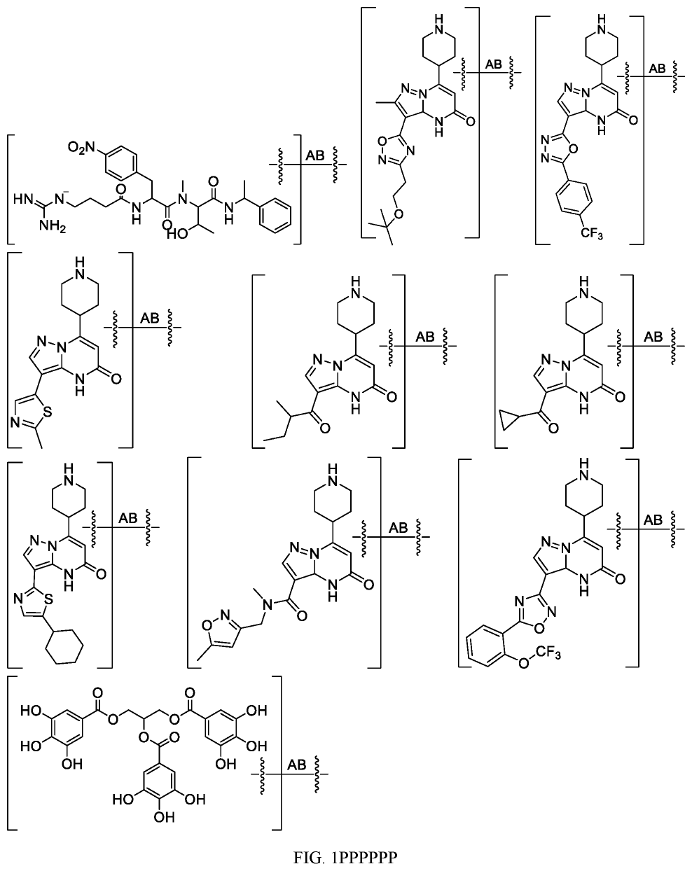

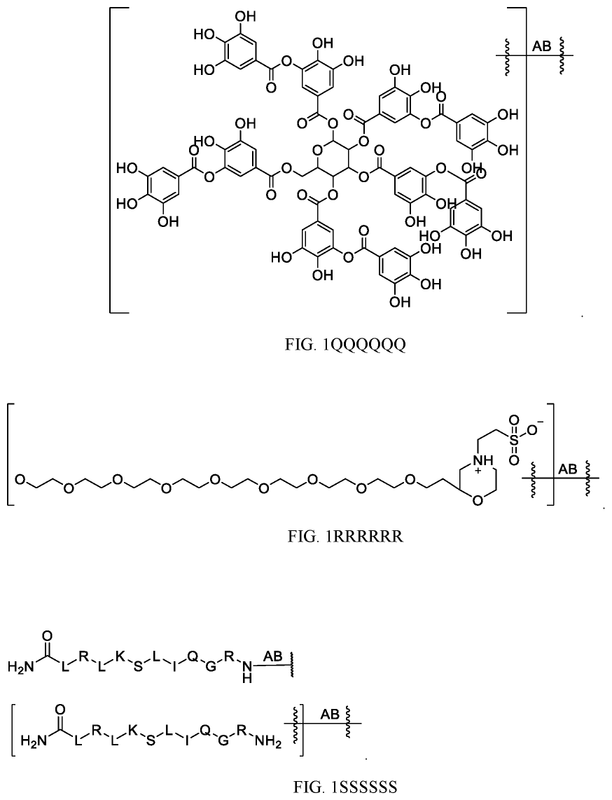

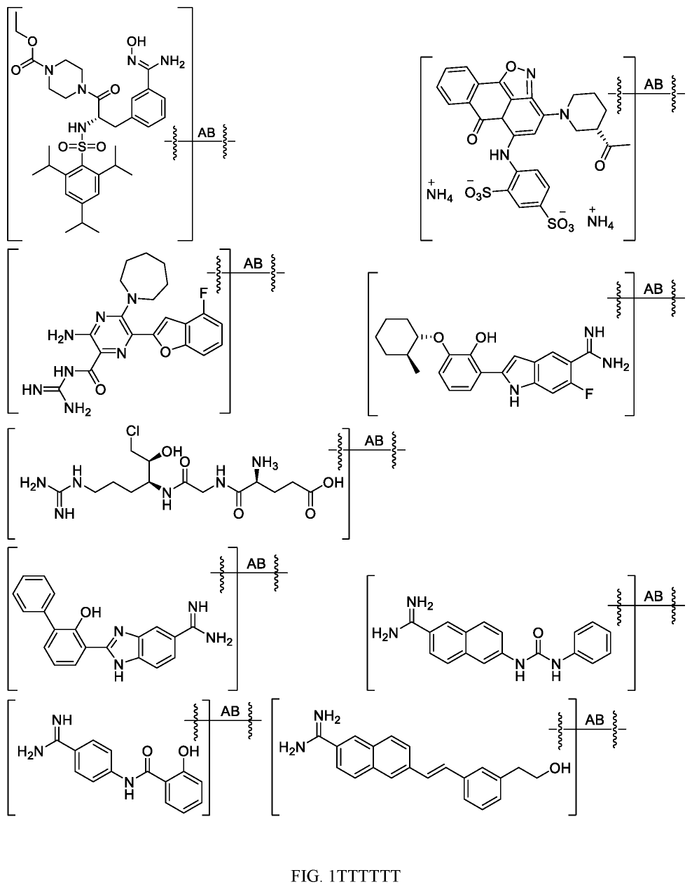

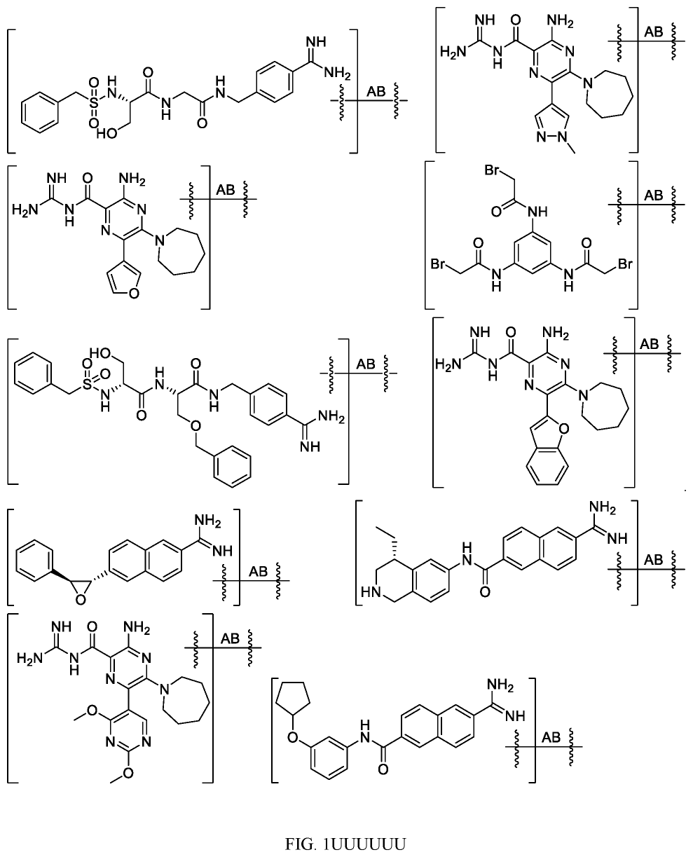

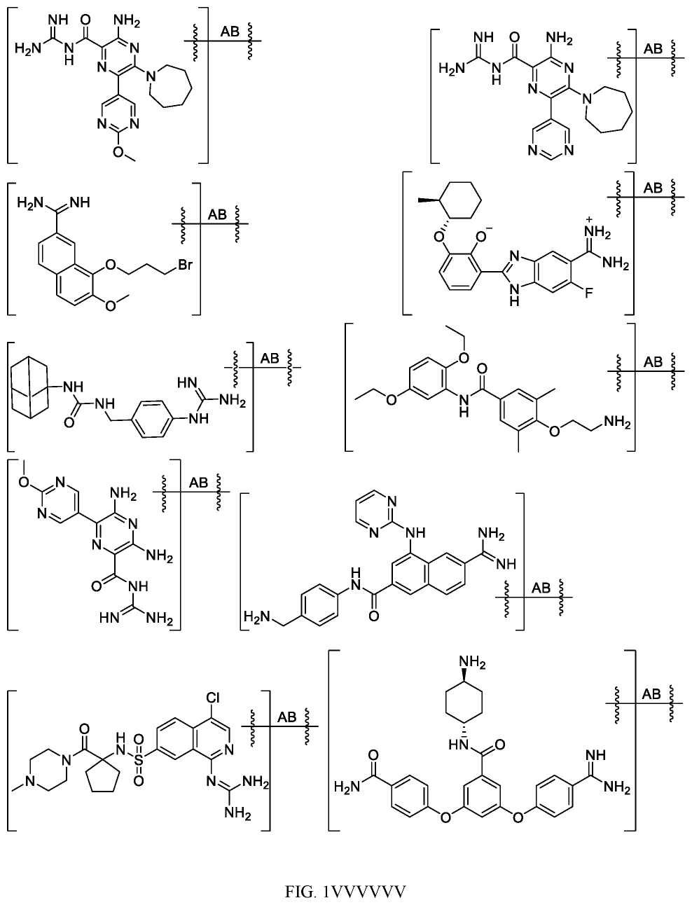

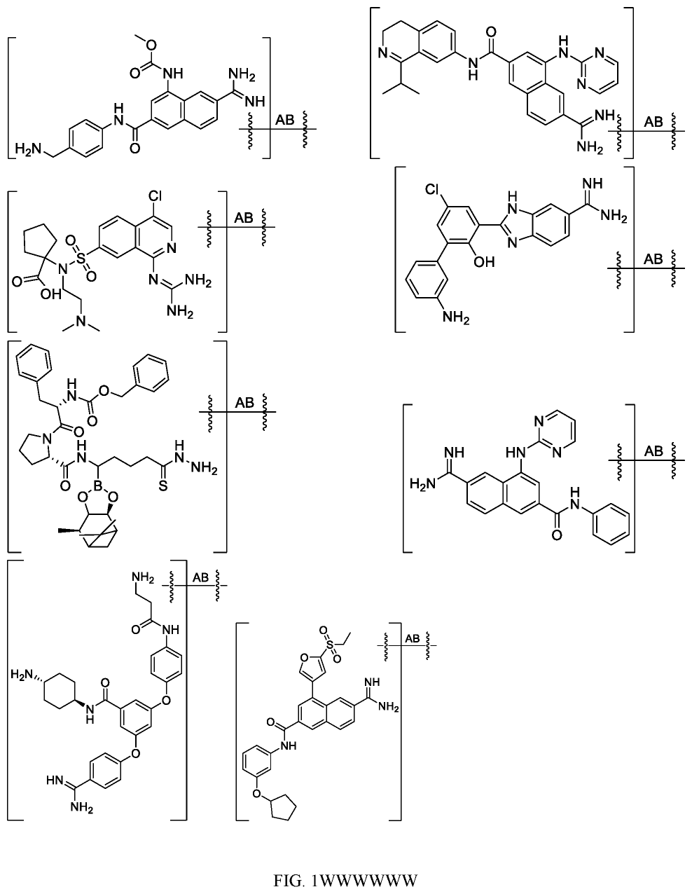

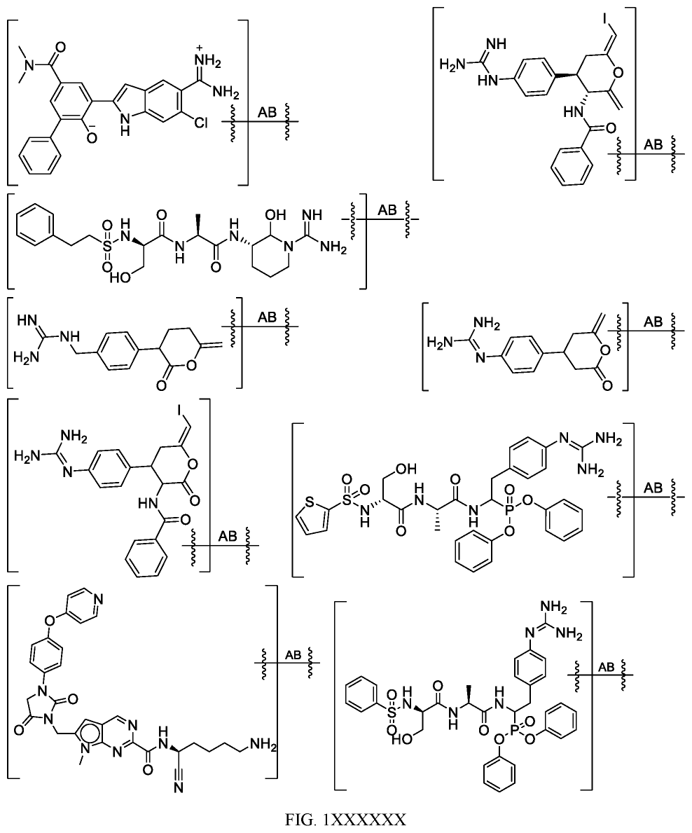

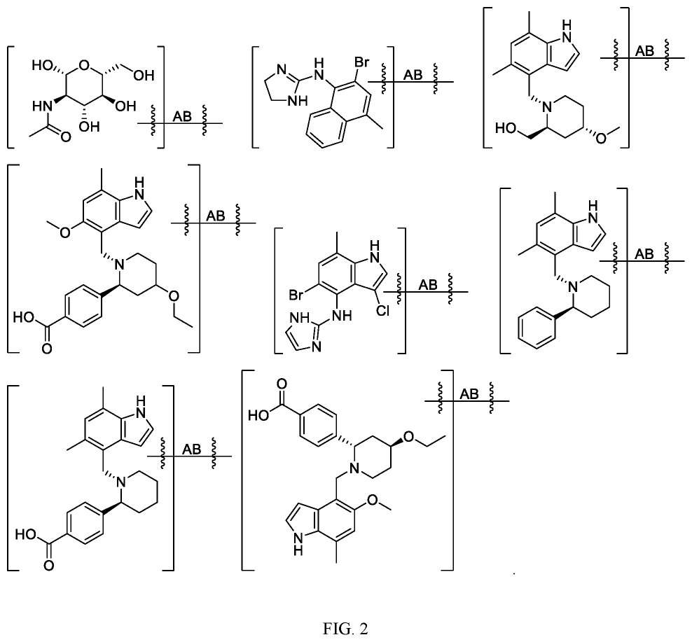

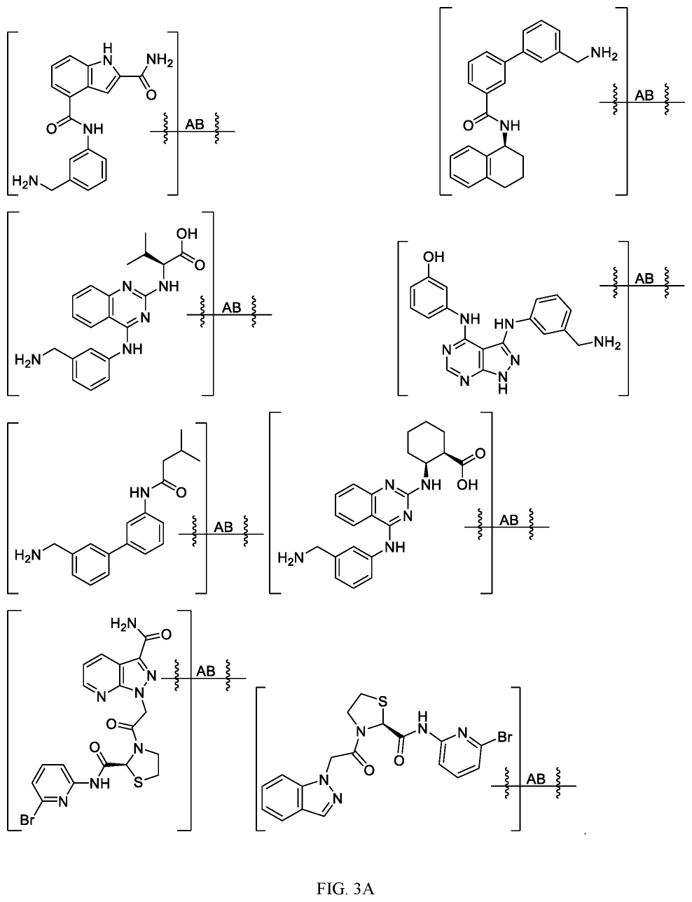

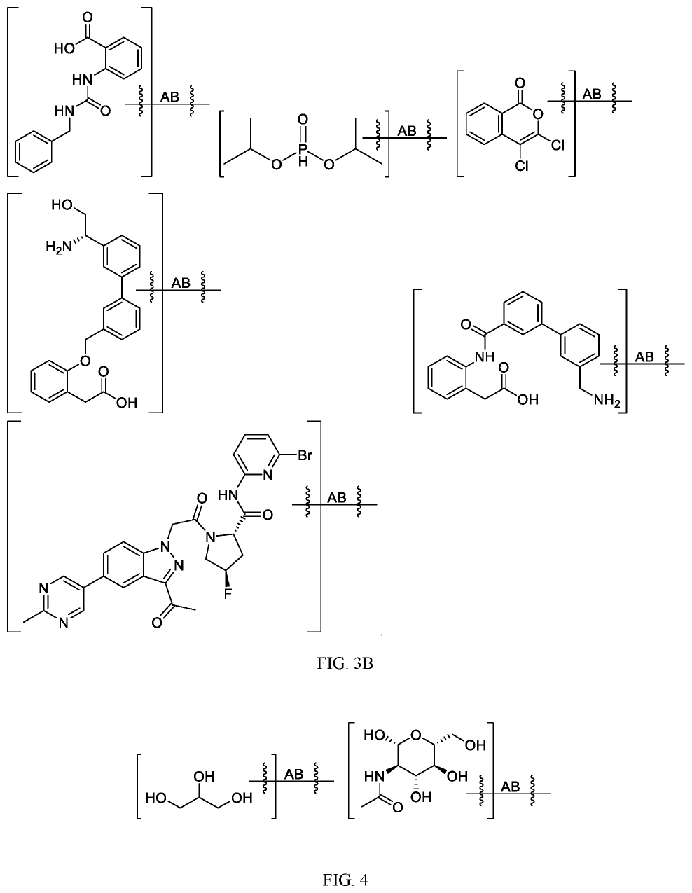

Representative TNF-α Targeting Ligands are provided in FIG. 1. Additional TNF-α Targeting Ligands can be found in, for example, U.S. Pat. No. 8,541,572; J Chem Inf Model. 2017 May 22; 57(5): 1101-1111; each of which is incorporated by reference herein.

In certain embodiments the TNF-alpha Targeting Ligand is selected from:

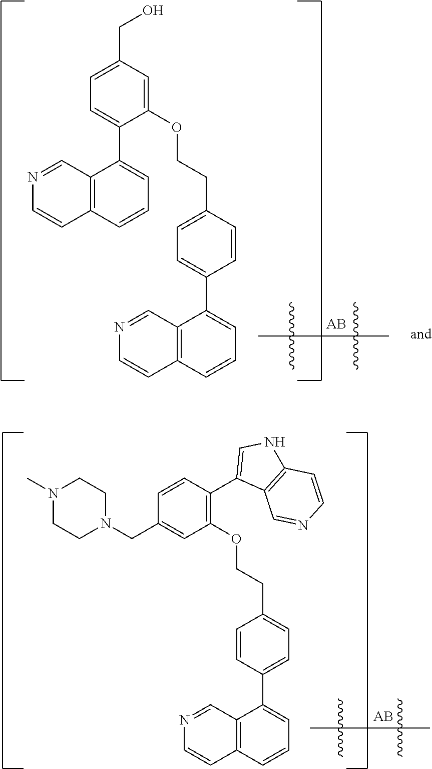

In certain embodiments the TNF-alpha Targeting Ligand is selected from:

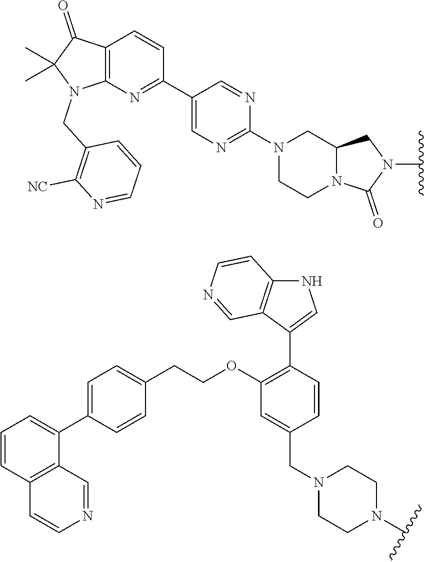 [SMILES: *N1C[C@@H]2CN(c3ncc(-c4ccc5c(n4)N(Cc4cccnc4C#N)C(C)(C)C5=O)cn3)CCN2C1=O ; *N1CCN(Cc2ccc(-c3c[nH]c4ccncc34)c(OCCc3ccc(-c4cccc5ccncc45)cc3)c2)CC1]

### IL-1

In some embodiments, the Target Extracellular Protein is human interleukin-1 (IL-1) (UniProtKB-P01584 (IL1B_HUMAN)). IL-1 is a potent proinflammatory cytokine. Initially discovered as the major endogenous pyrogen, induces prostaglandin synthesis, neutrophil influx and activation, T-cell activation and cytokine production, B-cell activation and antibody production, and fibroblast proliferation and collagen production. IL-1 promotes Th17 differentiation of T-cells, and Synergizes with IL12/interleukin-12 to induce IFNG synthesis from T-helper 1 (Th1) cells. IL-1 has been implicated in a number of auto-inflammatory and autoimmune disorders, including, but not limited to, Blau syndrome, cryopyrin-associated periodic syndromes, familial Mediterranean fever, Majeed syndrome; mevalonate kinase deficiency syndrome, pyogenic arthritis-pyoderma gangrenosum-acne syndrome, tumor necrosis factor receptor-associated periodic syndrome, Behcet's Disease, Sjogren's Syndrome, gout and chondrocalcinosis, periodic fever, aphthous stomatitis, pharyngitis, and cervical adenitis (or PFAPA) syndrome, rheumatoid arthritis, Type 2 diabetes mellitus, acute pericarditis, Chronic interstitial lung diseases (ILDs), Still's Disease,

The Protein Data Bank website provides the crystal structure of IL-1 searchable by 9ILB (Yu, B., et al., Proc Natl Acad Sci USA, 1999, 96 103-108); 1I1B (Finzel, B. C., et al., J Mol Biol., 1989, 209 779-791); and 3040 (Wang et al., Nat. Immunol., 2010, 11: 905-911); as well as the crystal structure of IL-1 bound to various compounds searchable by 4G6J (Blech, M., et al., J Mol Biol., 2013, 425 94-111); 5BVP (Rondeau e al., MAbs, 2015, 7 1151-1160); and 3LTQ (Barthelmes, K., et al., J Am Chem. Soc., 2011, 133 808-819). Additionally, Guy et al., provides insight into the crystal structure of a small antagonist peptide bound to interleukin-1 receptor type 1 (Guy et al., The Journal of Biological Chemistry, 2000, 275, 36927-36933).

Non-limiting examples of IL-1 direct or indirect inhibitors are described in FIG. 1. Additional IL-1 Targeting Ligands can be found in, for example, U.S. Pat. No. 9,694,015, each of which is incorporated herein by reference. Additional binding ligands include rilonacept or a binding fragment thereof (J Rheumatol. 2012; 39:720-727 (2012); and Canakinumab, or a binding fragment thereof (J Rheumatol. 2004; 31:1103-1111).

In certain embodiments the IL-1 Targeting Ligand is selected from

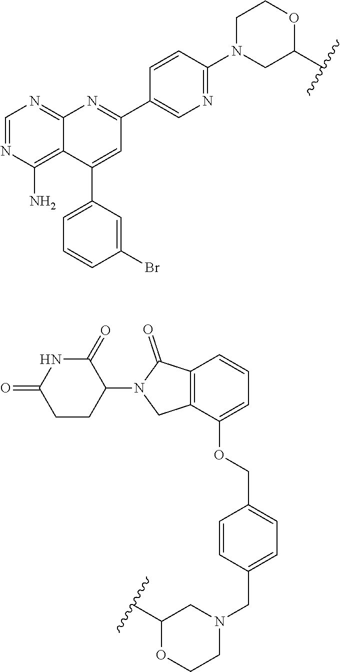 [SMILES: *C1CN(c2ccc(-c3cc(-c4cccc(Br)c4)c4c(N)ncnc4n3)cn2)CCO1 ; *C1CN(Cc2ccc(COc3cccc4c3CN(C3CCC(=O)NC3=O)C4=O)cc2)CCO1]

### IL-2

In some embodiments, the Target Extracellular Protein is human interleukin-2 (IL-2) (UniProtKB-P60568 (IL2_HUMAN)). IL-2 is a potent pro-inflammatory cytokine. IL-2 has been implicated in host versus graft rejection and other autoimmune disorders.

The Protein Data Bank website provides the crystal structure of IL-2 searchable by 1M4C and 1M47 (Arkin, M. R., et al., Proc. Natl. Acad. Sci. USA, 2003, 100: 1603-1608); as well as the crystal structure of IL-2 bound to various compounds searchable by 4NEJ and 4NEM (Brenke, R., et al.); 1QVN (Thanos, C. D., et al., Proc Natl Acad Sci USA, 2006, 103 15422-15427); 1PW6 and 1PY2 (Thanos, C. D., et al., J Am Chem Soc., 2003, 125 15280-15281); 1NBP (Hyde, J., et al., Biochemistry, 2003, 42 6475-6483); and 1M48, 1M49, 1M4A, 1M4B, and 1M4C (Arkin, M. R., et al., Proc Natl Acad Sci USA, 2003, 100 1603-1608). Additionally, Stauber, D. J., et al, provides insight into the crystal structure of the IL-2 signaling complex: paradigm for a heterotrimeric cytokine receptor (Stauber, D. J., et al., PNAS, 2006, 103(8), 2788-2793).

Representative IL-2 Targeting Ligands are provided in FIG. 1. Additional IL-2 Targeting Ligands can be found in, for example, U.S. Pat. Nos. 8,802,721; 9,682,976, 9,708,268; Eur J Med Chem 83: 294-306 (2014), J Med Chem 60: 6249-6272 (2017); Nature 450: 1001-1009 (2007); each of which is incorporated by reference herein.

In certain embodiments the IL-2 Targeting Ligand is selected from

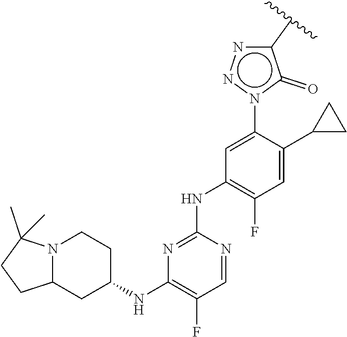

### IFN-γ

In some embodiments, the Target Extracellular Protein is human interferon-γ (IFN-γ) (UniProtKB-Q14609 (Q14609_HUMAN)). IFN-γ is a immunoregulatory cytokine. IFN-γ has been implicated in a number of autoimmune disorders, including, but not limited to rheumatoid arthritis, multiple sclerosis (MS), corneal transplant rejection, and various autoimmune skin diseases such as psoriasis, alopecia areata, vitiligo, acne vulgaris, and others.

The Protein Data Bank website provides the crystal structure of IFN-γ searchable by 1HIG (Ealick, S. E., et al., Science 252, 1991, 698-702); as well as the crystal structure of IFN-γ bound to various compounds searchable by 6E3K and 6E3L (Mendoza, J. L., et al., Nature, 2019, 567 56-60). Additionally, Randal et al., provides insight into the structure and activity of a monomeric interferon-γ: α-chain receptor signaling complex (Randal, M., et al., Structure, 2001, 9(2), 155-163).

Representative IFN-γ Targeting Ligands are described in FIG. 1. Additional IFN-γ Targeting Ligands can be found in, for example, J Med Chem 57: 4511-20 (2014); which is incorporated by reference herein.

### Vascular Epithelial Growth Factor (VEGF)

In some embodiments, the Target Extracellular Protein is human vascular epithelial growth factor (VEGF) (UniProtKB-P15692 (VEGFA_HUMAN)). VEGF is a growth factor active in angiogenesis, vasculogenesis, and endothelial cell growth. VEGF induces endothelial cell proliferation, promotes cell migration, inhibits apoptosis and induces permeabilization of blood vessels. VEGF has been implicated in the vascularization and angiogenesis of tumors.

The Protein Data Bank website provides the crystal structure of VEGF searchable by 3QTK (Mandal, K., et al., Angew Chem Int Ed Engl., 2011, 50 8029-8033); and 4KZN (Shen et al.); as well as the crystal structure of VEGF bound to various compounds searchable by 504E (Lobner, E., et al., MAbs, 2017, 9 1088-1104); 4QAF (Giese, T., et al.,); 5DN2 (Tsai, Y. C. I., et al., FEBS, 2017, J 283 1921-1934); 4GLS (Mandal, K., et al., Proc Natl Acad Sci USA, 2012, 109 14779-14784); and 1KMX (Stauffer, M. E. et al., J Biomol NMR, 2002, 23 57-61). Additionally, Mueller, Y. A., et al, provides insight into the Crystal structure and functional mapping of the kinase domain receptor binding site of VEGF (Mueller, Y. A., et al., Proc Natl Acad Sci USA., 1997 Jul. 8; 94(14): 7192-7197).

Representative VEGF Targeting Ligands are provided in FIG. 1. Additional VEGF Targeting Ligands include, but are not limited to, the peptide NH₂-LREFCEWEWMVHIDCNPEV (SEQ ID: 2) (Biochemistry 1998, 37, 17754-177764). Additional VEGF Targeting Ligands are provided in, for example, J Med Chem 57: 3011-29 (2014), U.S. Pat. Nos. 9,884,843, 9,446,026, J Med Chem 53: 1686-99 (2010), J Med Chem 48: 8229-36 (2005), J Nat Prod 76: 29-35 (2013), each of which is incorporated herein by reference. All references cited herein are incorporated by reference.

### Transforming Growth Factor-β1 (TGF-β1)

In some embodiments, the Target Extracellular Protein is human transforming growth factor-01 (TGF-β1) (UniProtKB-P01137 (TGFB1_HUMAN)). TGF- 31 is a multifunctional protein that regulates the growth and differentiation of various cell types and is involved in various processes, such as normal development, immune function, microglia function and responses to neurodegeneration. TGF- 31 can promote either T-helper 17 cells (Th17) or regulatory T-cells (Treg) lineage differentiation in a concentration-dependent manner. TGF- 01 expression in the tumor microenvironment has been associated with a poor prognosis, and is implicated in TGF-β1 mediated tumor suppression via T-cell exclusion. TGF-β1 expression has also been implicated in hematological malignancies and fibrosis.

The Protein Data Bank website provides the crystal structure of TGF-β1 searchable by 5E8S, 5E8T, and 5E8U (Tebben, A. J., et al., Acta Crystallogr D Struct Biol., 2016, 72 658-674); 2L5S (Zuniga, J. E., et al, J Mol Biol., 2011, 412 601-618); and 2PJY (Groppe, J., et al., Mol Cell, 2008, 29 157-168); as well as the crystal structure of TGF-β1 bound to various compounds searchable by 5QIK, 5QIL and 5QIM, (Zhang, Y., et al., ACS Med Chem Lett., 2018, 9 1117-1122); 6B8Y (Harikrishnan, L. S., et al., Bioorg Med Chem., 2018, 26 1026-1034); 5E8W, 5E8X, 5E8Z, and 5E90 (Tebben, A. J., et al., Acta Crystallogr D Struct Biol., 2016, 72 658-674); 3TZM (Ogunjimi, A. A. et al., Cell Signal, 2012, 24 476-483); 2X7O (Roth, G. J., et al., J Med Chem., 2010, 53 7287); 3KCF (Guckian, K., et al., Bioorg Med Chem Lett., 2010, 20 326-329); 3FAA (Bonafoux, D., et al., Bioorg Med Chem Lett., 2009, 19 912-916); 1VJY (Gellibert, F, J., et al., J Med Chem., 2004 47 4494-4506); and 1PY5 (Sawyer, J. S., et al., Bioorg Med Chem Lett., 2004, 14 3581-3584). Additionally, Hinck et al., provides insight into the structural studies of the TGF-βs and their receptors and further insight into evolution of the TGF-P superfamily (Hinck, A., FEBS, 2012, 586(14), 1860-1870).

Representative TGF- R 1 Targeting Ligands are provided in FIG. 1. In some embodiments, the TGF-β1 Targeting Ligand is the peptide KRFK peptide (J. Biol. Chem. Vol. 274 (No. 19) pp. 13586-13593 (1999) (incorporated herein by reference). Additional TGF- 31 Targeting Ligands are provided in, for example, Bioorg Med Chem Lett 21: 5642-5 (2011), which is incorporated herein by reference.

### Proprotein Convertase Subtilisin/Kexin Type 9 (PCSK-9)

In some embodiments, the Target Extracellular Protein is human proprotein convertase subtilisin/kexin type 9 (PCSK-9) (UniProtKB-Q8NBP7 (PCSK9_HUMAN)). PCSK-9 is a crucial player in the regulation of plasma cholesterol homeostasis. PCSK-9 binds to low-density lipid receptor family members: low density lipoprotein receptor (LDLR), very low-density lipoprotein receptor (VLDLR), apolipoprotein E receptor (LRP1/APOER) and apolipoprotein receptor 2 (LRP8/APOER2), and promotes their degradation in intracellular acidic compartments. It acts via a non-proteolytic mechanism to enhance the degradation of the hepatic LDLR through a clathrin LDLRAP1/ARH-mediated pathway, and may prevent the recycling of LDLR from endosomes to the cell surface or direct it to lysosomes for degradation. PCSK-9 has been implicated in high blood cholesterol and the development of cardiovascular disease.

The Protein Data Bank website provides the crystal structure of PCSK-9 searchable by 2P4E (Cunningham, D., et al., Nat Struct Mol Biol., 2007, 14 413-419); as well as the crystal structure of PCSK-9 bound to various compounds searchable by 3BPS (Kwon, H. J., et al., Proc Natl Acad Sci USA, 2008, 105 1820-1825); 6U26, 6U2N, 6U2P, 6U36, 6U38, and 6U3X (Petrilli, W. L., et al., Cell Chem Biol., 2019, 27 32-40.e3); 50CA (Gustafsen, C., et al., Nat Commun., 2017, 8 503-503); 4NE9 (Schroeder, C. I., et al., Chem Biol., 2014, 21 284-294); 40V6 (Mitchell, T., et al., J Pharmacol Exp Ther., 2014, 350 412-424); and 4NMX (Zhang, Y., et al., J Biol Chem., 2014, 289 942-955). Additionally, Piper et al., provides insight into the crystal structure of PCSK9 (Piper, D. E., et al., Structure, 2007, 15(5), 545-52).

Representative PCSK-9 Targeting Ligands are provided in FIG. 1. In some embodiments, the PCSK-9 Targeting Ligand is the peptide TVFTSWEEYLDWV (SEQ ID: 3) (J. Bio. Chem. 2014 January; 289(2):942-955, incorporated herein by reference). Additional PCSK-9 Targeting Ligands are provided in, for example, U.S. Pat. No. 9,227,956, J Biol Chem 289: 942-55 (2014), each of which is incorporated by reference herein.

In certain embodiments the PCSK-9 ligand is any PCSK-9 ligand described in WO2021/156792 which is incorporated by reference.

In certain embodiments a compound is provided of Formula

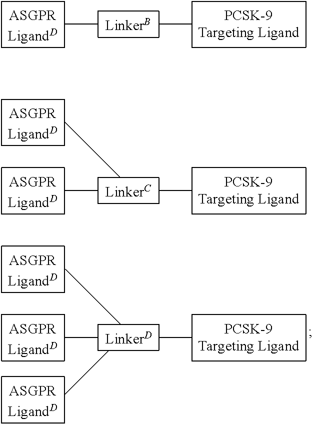

or a pharmaceutically acceptable salt thereof,
wherein

ASGPR Ligand^{D} is selected from ASGPR Ligand^{A}, and

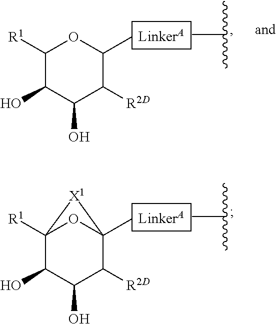

R^{2D} is selected from
(i) aryl, heterocycle, and heteroaryl containing 1 or 2 heteroatoms independently selected from N, O, and S, each of which aryl, heterocycle, and heteroaryl is optionally substituted with 1, 2, 3, or 4 substituents;

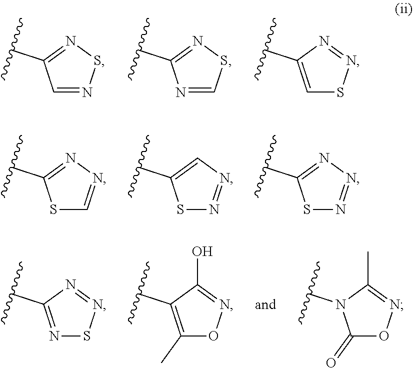

(iii) —NR⁸—S(O)—R³, —NR⁸—C(S)—R³, —NR⁸—S(O)(NR⁶)—R³, —N═S(O)(R³)₂, —NR⁸C(O)NR⁹S(O)₂R³, —NR⁸—S(O)₂—R¹⁰, and —NR⁸—C(NR⁶)—R³ each of which is optionally substituted with 1, 2, 3, or 4 substituents; and
(iv) hydrogen, R¹⁰, alkyl-C(O)—R³, —C(O)—R³, alkyl, haloalkyl, —OC(O)R³, and —NR⁸—C(O)R¹⁰;
R¹⁰ is selected from alkenyl, allyl, alkynyl, —NR⁶-alkenyl, —O-alkenyl, —NR⁶-alkynyl, —NR⁶-heteroaryl, —NR⁶-aryl, —O-heteroaryl, —O-aryl, —O-alkynyl, aryl, alkyl-NR⁸—C(O)—R³, alkyl-aryl, alkyl-heteroaryl with 1, 2, or 4 heteroatoms, alkyl-cyano, alkyl-OR⁶, alkyl-NR⁶R⁸, NR⁸—NR⁶—C(O)R³, NR⁸—S(O)₂—R³, each of which R¹⁰ is optionally substituted with 1, 2, 3, or 4 substituents;
PCSK-9 Targeting Ligand is any PCSK-9 ligand described in WO2021/156792.

Non-limiting examples of PCSK-9 Targeting Ligands that can be used in any of the formulas of the present invention include:

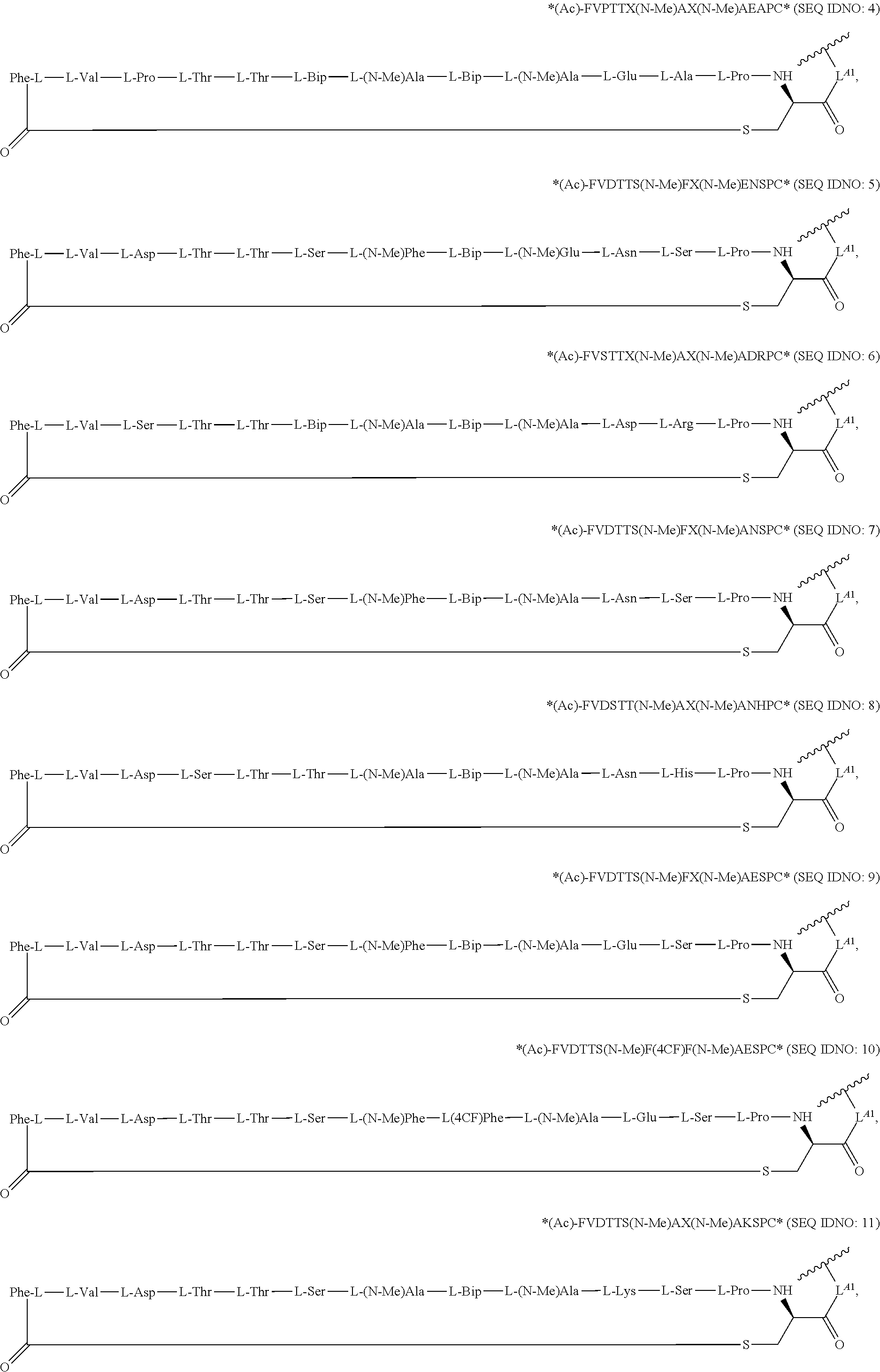

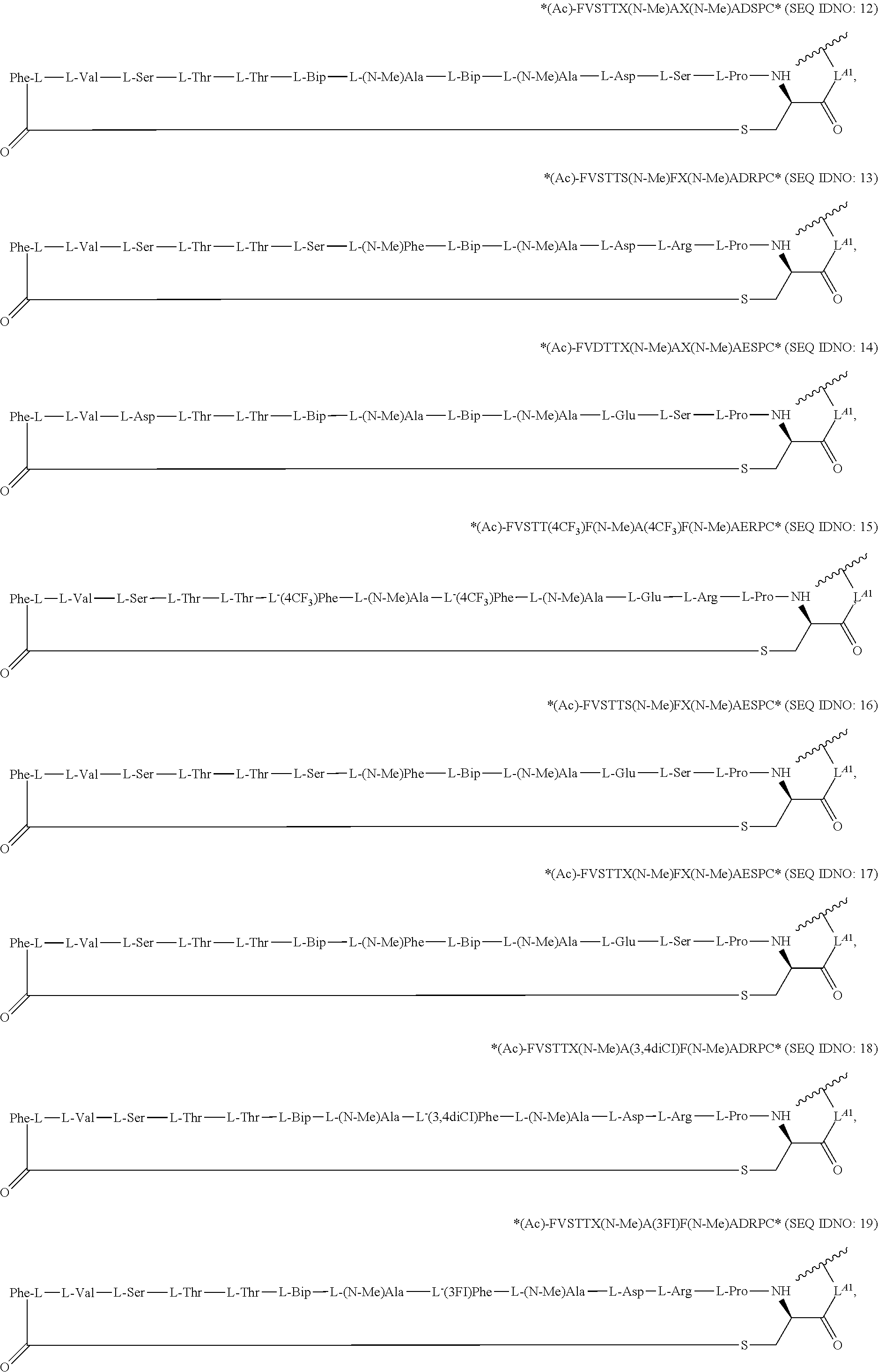

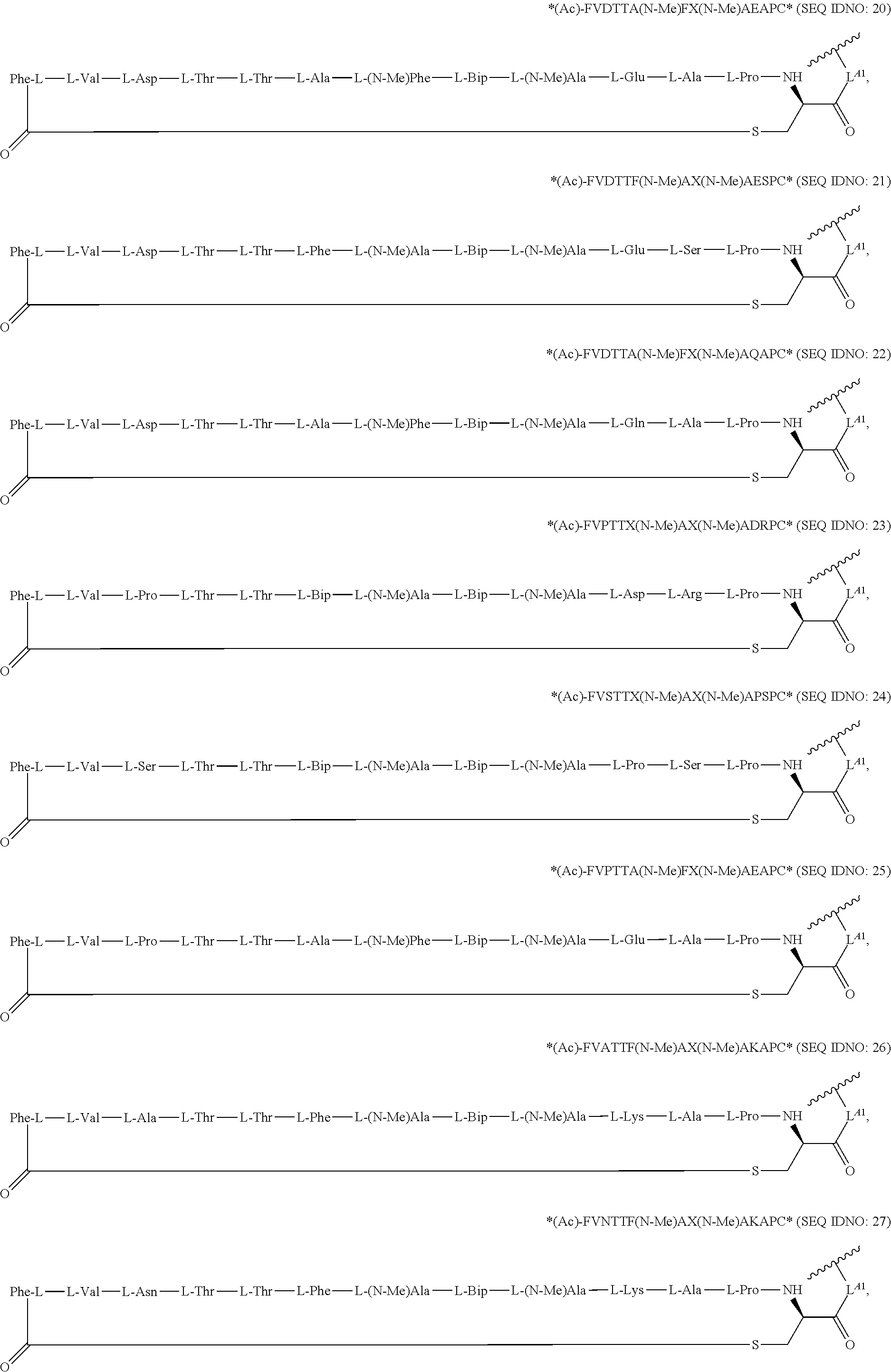

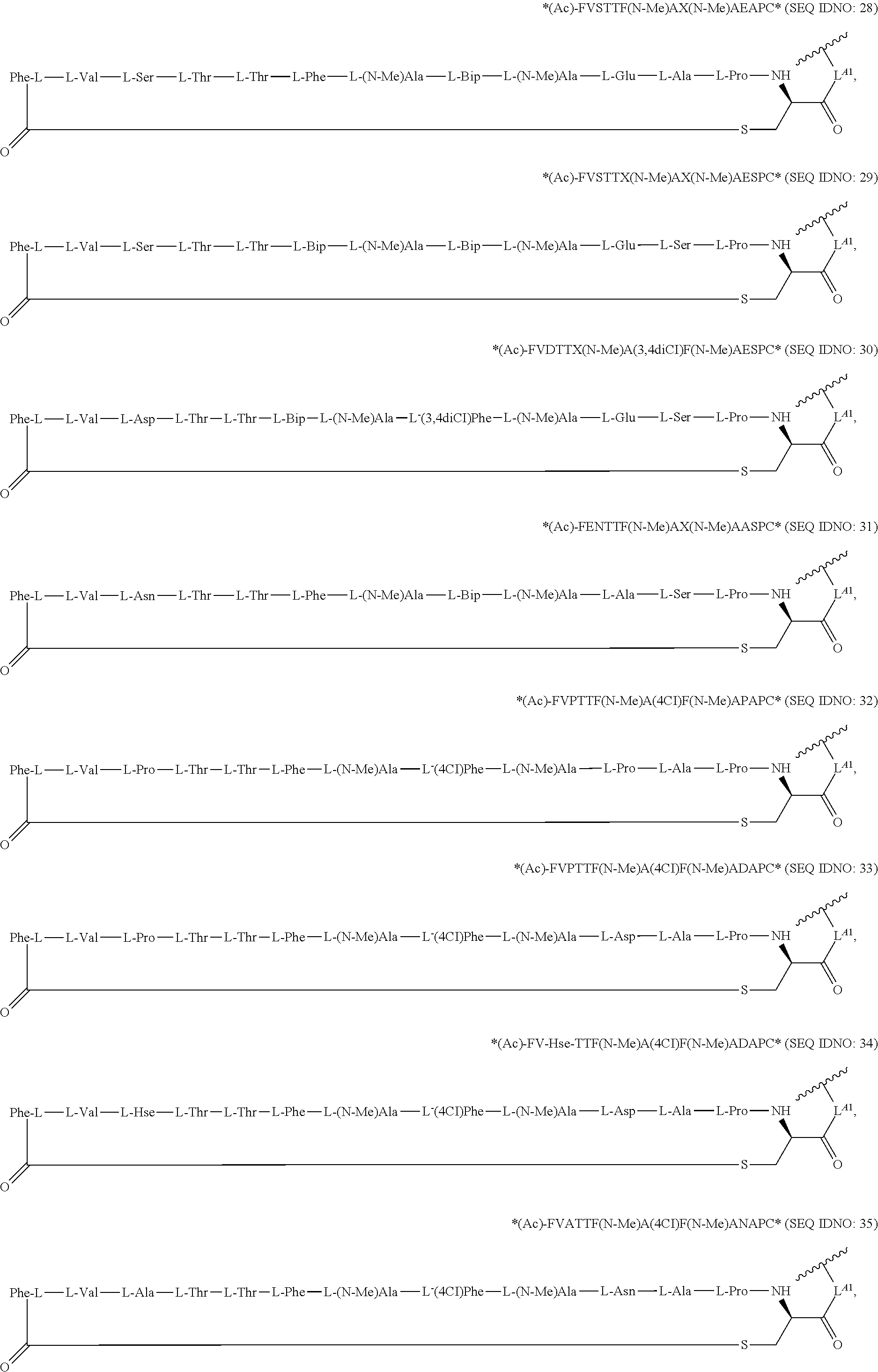

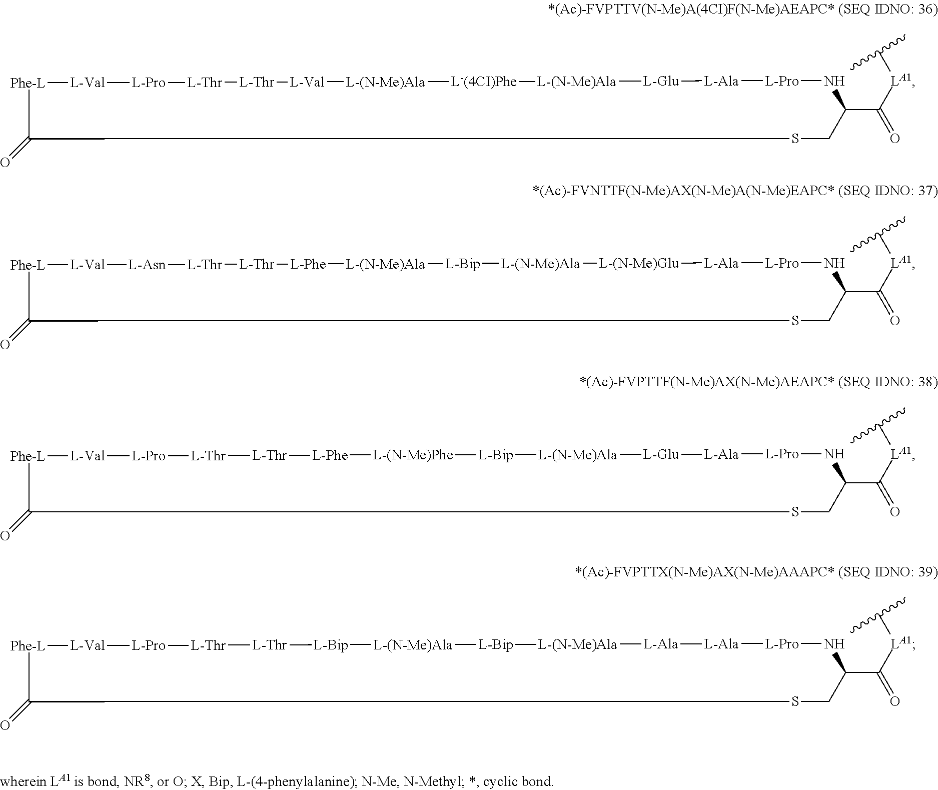

In certain embodiments the PCSK9 Targeting Ligand is a compound of Formula:

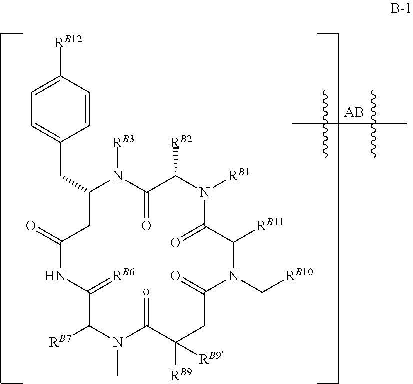

wherein,
R^{B1} is H;
R^{B2} is (C₁-C₆)alkoxy, -L^{B1}-, or (C₁-C₆)alkyl, substituted with —C(═O)OH;
R^{B3} is H or (C₁-C₆)alkyl;
R^{B6} is H, (C₁-C₆)alkyl or L^{B1};
R^{B7} is H, (C₁-C₆)alkyl or L^{B1};
or R^{B6} and R^{B7} together with the carbon atoms to which they are attached form a (C₃-C₇)cycloalkyl;
R^{B9} is H or (C₁-C₆)alkyl, optionally substituted with one or more R^{B27};
R^{B9′} is H or (C₁-C₆)alkyl;
R^{B10} is (C₆-C₁₀)aryl substituted with OR^{B13} and optionally substituted with one or more R^{B14};
R¹¹ is (C₁-C₆)alkyl or L^{B1};
R^{B12} is halogen, (C₁-C₆)alkyl, (C₁-C₆)alkoxy, (C₁-C₆)haloalkyl, (C₁-C₆)haloalkoxy, —OH, or CN;
R^{B13} is (C₆-C₁₀)aryl substituted with R^{B16}.
each R^{B14} is independently at each occurrence halogen, (C₁-C₆)alkyl, (C₁-C₆)alkoxy, (C₁-C₆)haloalkyl, (C₁-C₆)haloalkoxy, oxo, —OH, or CN;
R^{B16} is 5- to 7-membered heteroaryl comprising 1-3 heteroatoms selected from N, O, and S, optionally substituted with one or more R^{B26};
each R^{B26} is independently at each occurrence (C₁-C₆)alkyl optionally substituted with one or more R^{B29};
each R^{B27} is independently at each occurrence (C₆-C₁₀)aryl;
each R^{B29} is independently at each occurrence —NR^{B31}R^{B32} or 4- to 7-membered heterocyclyl comprising 1-3 heteroatoms selected from N, O, and S;
each R^{B31} is independently selected from Hand (C₁-C₆)alkyl;
each R^{B32} is independently selected from Hand (C₁-C₆)alkyl;
L^{B1} is —(CH₂)ₚNH—*, where the * of L^{B1} indicates the point of attachment to Linker (L_{A}), and where at least one of R^{B11}, R^{B6} or R^{B7} is -L^{B1}-; and
n is 1.

In certain embodiments the PCSK9 Targeting Ligand is a compound of Formula:

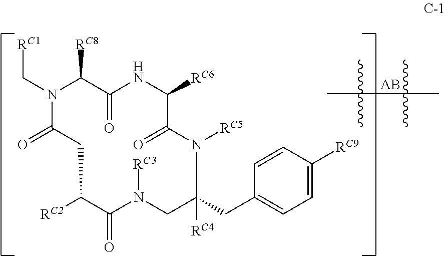

wherein,
R^{C1} is (C₆-C₁₀)aryl substituted with —OR^{C10} and one or more R^{C11};
R^{C2} is H, (C₁-C₆)alkyl, -L^{C1} or (C₃-C₉)carbocyclyl, wherein the alkyl is substituted with one R^{C18}, and the carbocyclyl is substituted with one or more R^{C19};
R^{C3} is H or (C₁-C₆)alkyl;
R^{C4} is H or (C₁-C₆)alkyl; or
R^{C3} and R^{C4} together with the atoms to which they are attached form a 5- to 7-membered heterocyclyl ring comprising 1-3 heteroatoms selected from N, O, and S;
R^{C5} is H or (C₁-C₆)alkyl;
R^{C6} is (C₁-C₆)alkyl, or -L^{C1}, wherein the alkyl is optionally substituted with one or more substituents each independently selected from —OH or (C₁-C₆)alkoxy;
R^{C8} is H, (C₁-C₆)alkyl, or -L^{C1},
R^{C9} is halogen;
R^{C10} is (C₆-C₁₀)aryl substituted with one R^{C22};
each R^{C11} is independently at each occurrence halogen, (C₁-C₆)alkyl, (C₁-C₆)alkoxy, (C₁-C₆)haloalkyl, (C₁-C₆)haloalkoxy, —OH, or CN;
R^{C18} is (C₆-C₁₀)aryl;
each R^{C19} is independently at each occurrence halogen, (C₁-C₆)alkyl, (C₁-C₆)alkoxy, (C₁-C₆)haloalkyl, (C₁-C₆)haloalkoxy, —OH, or CN;
R^{C22} is 5- or 6-membered heteroaryl comprising 1-3 heteroatoms selected from N, 0, and S, substituted with one or more R^{C23};
each R^{C23} is independently at each occurrence (C₁-C₆)alkyl, optionally substituted with —NR^{C24}R^{C25} or a 4- to 7-membered heterocyclyl comprising 1-3 heteroatoms selected from N, 0, and S;
R^{C24} is H, (C₁-C₆)alkyl;
R^{C25} is H, (C₁-C₆)alkyl,
L^{C1} is —(CH₂)ₚNH—*, where the * of L^{C1} indicates the point of attachment to Linker (L_{A}), and where at least one of R^{C2}, R^{C6} or R^{C8} is -L^{C1}; and
p is 1, 2, 3, 4, 5, or 6.

In certain embodiments the PCSK-9 Targeting Ligands that can be used in any of the formulas of the present invention include:

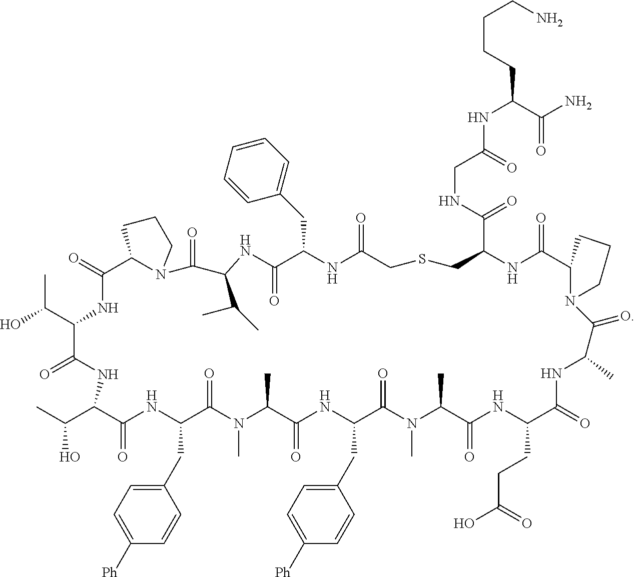 [SMILES: CC(C)[C@@H]1NC(=O)[C@H](Cc2ccccc2)NC(=O)CSC[C@@H](C(=O)NCC(=O)N[C@@H](CCCCN)C(N)=O)NC(=O)[C@@H]2CCCN2C(=O)[C@H](C)NC(=O)[C@H](CCC(=O)O)NC(=O)[C@H](C)N(C)C(=O)[C@H](Cc2ccc(-c3ccccc3)cc2)NC(=O)[C@H](C)N(C)C(=O)[C@H](Cc2ccc(-c3ccccc3)cc2)NC(=O)[C@H]([C@@H](C)O)NC(=O)[C@H]([C@@H](C)O)NC(=O)[C@@H]2CCCN2C1=O]

In certain embodiments the PCSK-9 Targeting Ligands that can be used in any of the formulas of the present invention include:

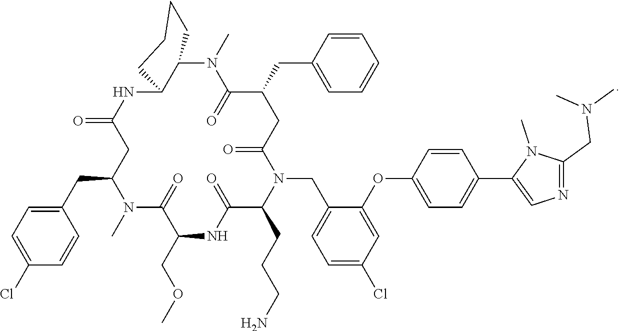

In certain embodiments the compound of the present invention is selected from

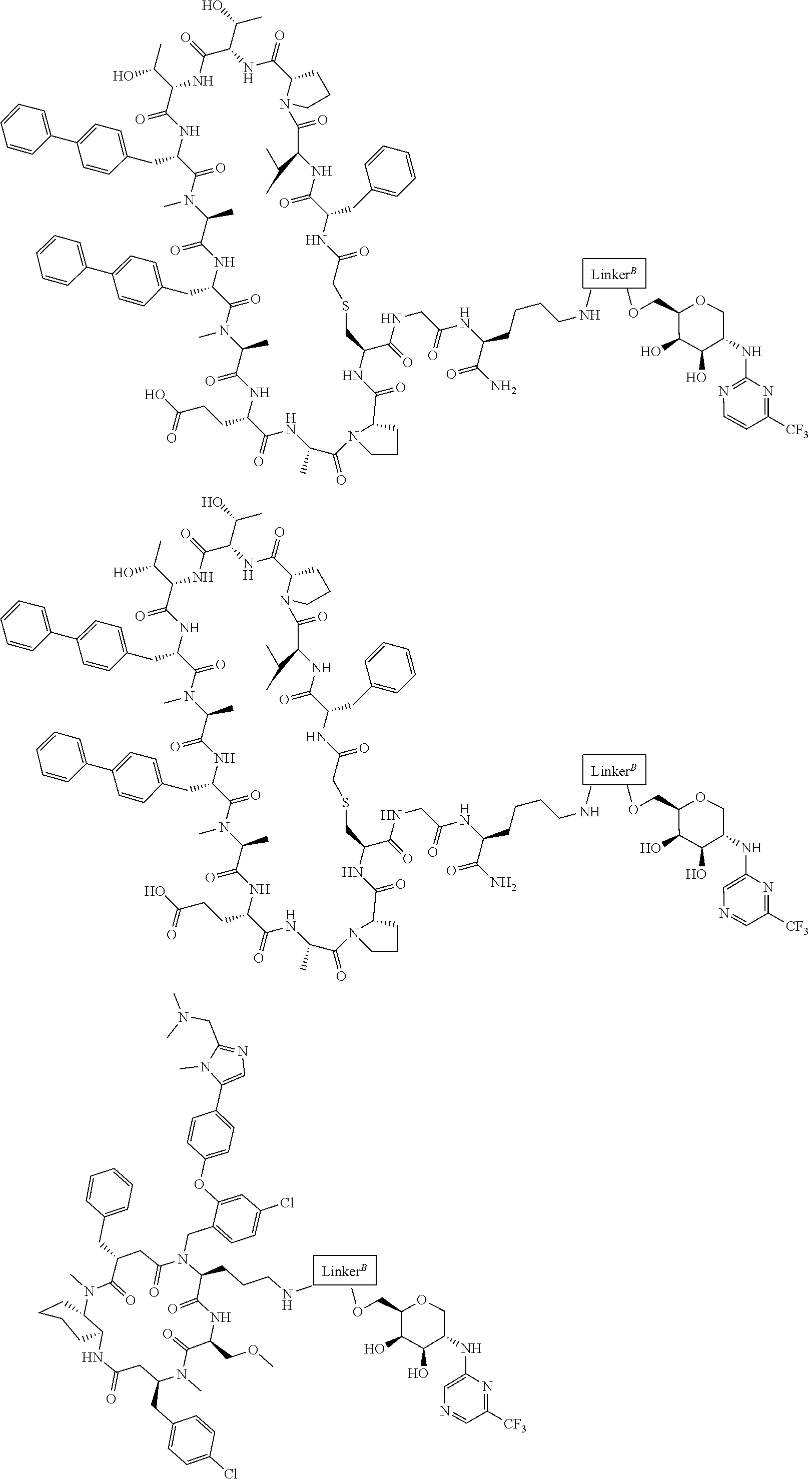

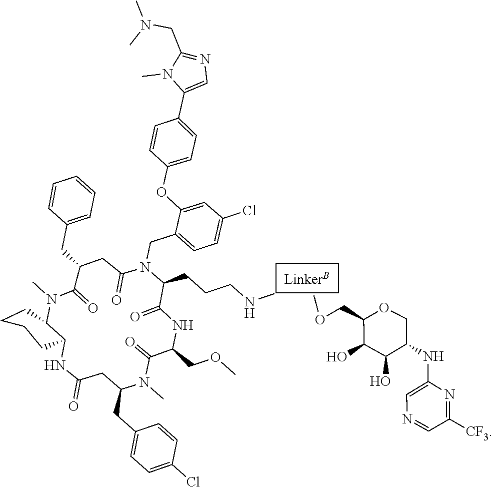

In certain embodiments the compound of the present invention is selected from

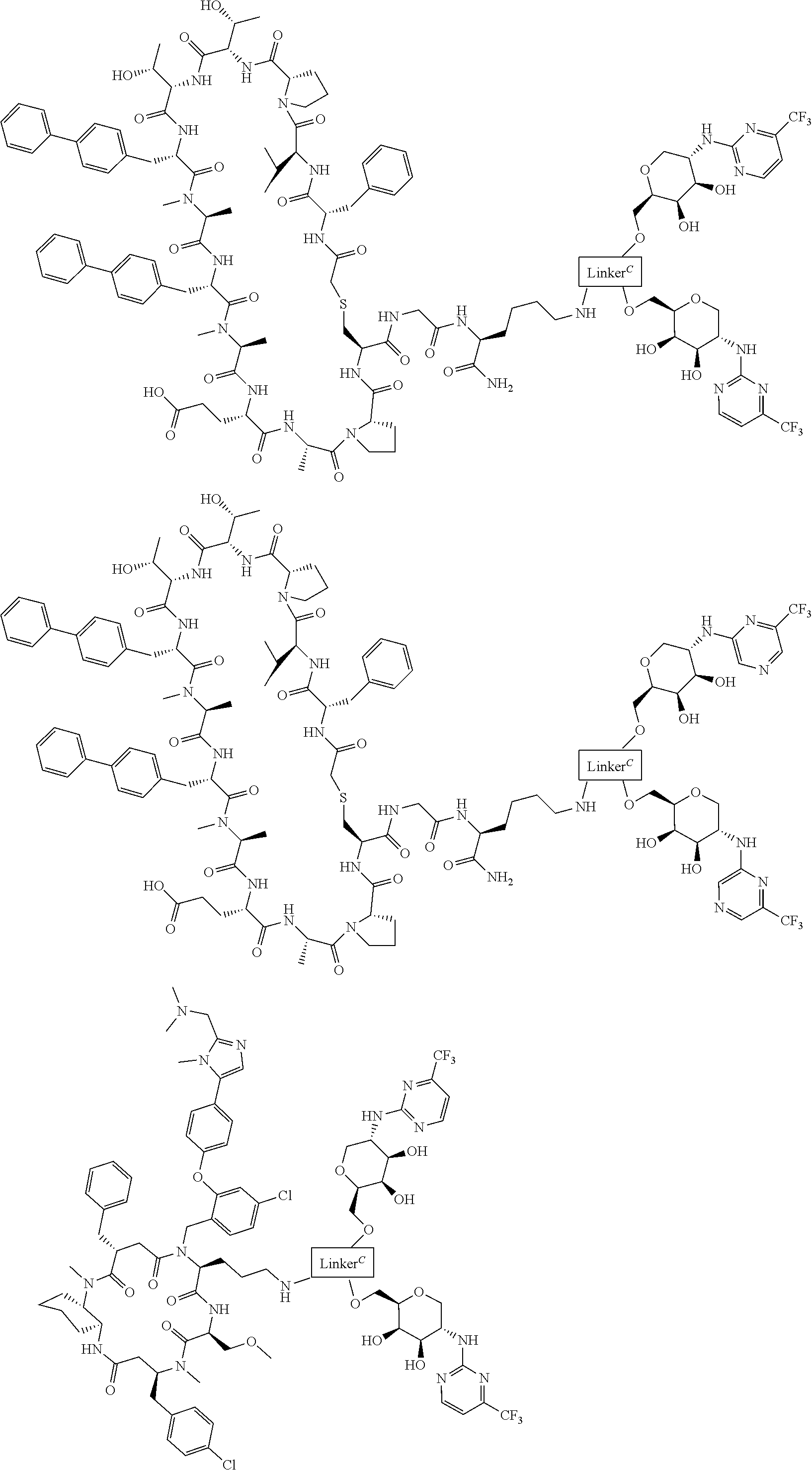

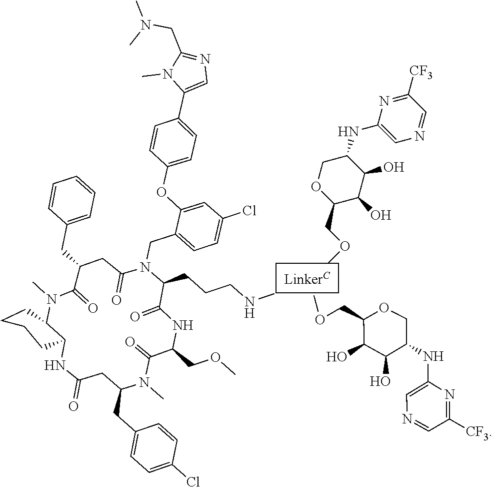

In certain embodiments the PCSK9 Targeting Ligand is selected from:

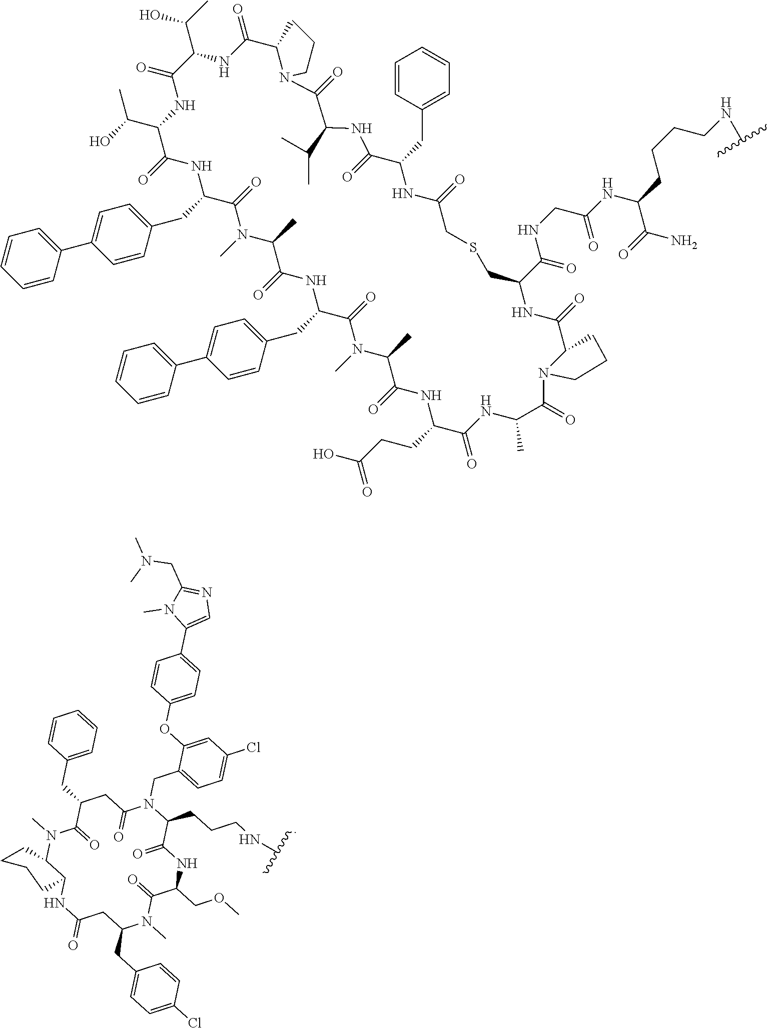

### FHR3

The human complement factor H-related protein 3 (FHR-3) belongs to the complement factor H (FH)-family. Factor H (FH), a major negative regulator of alternative complement pathway activation, belongs to a family that also includes five other related family members thought to have arisen from nonallelic homologous recombination and interlocus gene conversion including: complement factor H-related protein 1 (FHR1), complement factor H-related protein 2 (FHR2), complement factor H-related protein 3 (FHR3), complement factor H-related protein 4 with isoforms 4A and 4B (FHR4A and FHR4B) and complement factor H-related protein 5 (FHR5).

FHR3, unlike factor H, lacks the complement regulatory domains essential for complement inactivation and also competes with factor H, resulting in complement over-activation. Thus, the present invention provides compounds for use in modulating the concentration of complement factor H-proteins, specifically FHR3, to remove factor H's competitor and thereby restore factor H-mediated regulation to treat disorders caused by excessive complement activation.

Due to the central role that factor H plays in the regulation of complement, there are many clinical implications arising from aberrant FH activity. Loss of function mutation in factor H increase susceptibility to the renal diseases, atypical hemolytic uremic syndrome (aHUS) and dense deposit disease (ODD), whilst polymorphic variation of complement factor H has been strongly associated with important human diseases, including age-related macular degeneration (AMO) and meningococcal sepsis (Clin Exp Immunol 151(2):210-230; Immunobiology 217(11):1034-1046).

In certain embodiment, the invention provides the use in the treatment of a FHR3 mediated disease or disorder.

In certain embodiments, the FHR3 mediated disease or disorder is a complement-related diseases, disorders of complement dysregulation, autoimmune diseases, kidney disease, retinal degenerative diseases, Rheumatic Diseases, associated degenerative diseases, autoimmune renal disease, dense deposit disease (ODD), and systemic autoimmune diseases.

In certain embodiments, nonlimiting examples of FHR3 mediated diseases or disorders include nephropathy, age-related macular degeneration, atypical hemolytic uremic syndrome (aHUS), autoimmune form of hemolytic uremic syndrome, hepatocellular carcinoma (HCC), C3 glomerulopathy, paroxysmal nocturnal hemoglobinuria, Polymyalgia rheumatica, rheumatoid arthritis, meningococcal sepsis, and SLE (Systemic lupus erythematosus).

In certain embodiments, the present invention provides compounds that utilize receptor mediated endocytosis to eliminate or decrease level of complement factor H-related protein 3 (FHR3) from the plasma.

In certain embodiments, the FHR3 Targeting Ligand is selected from:

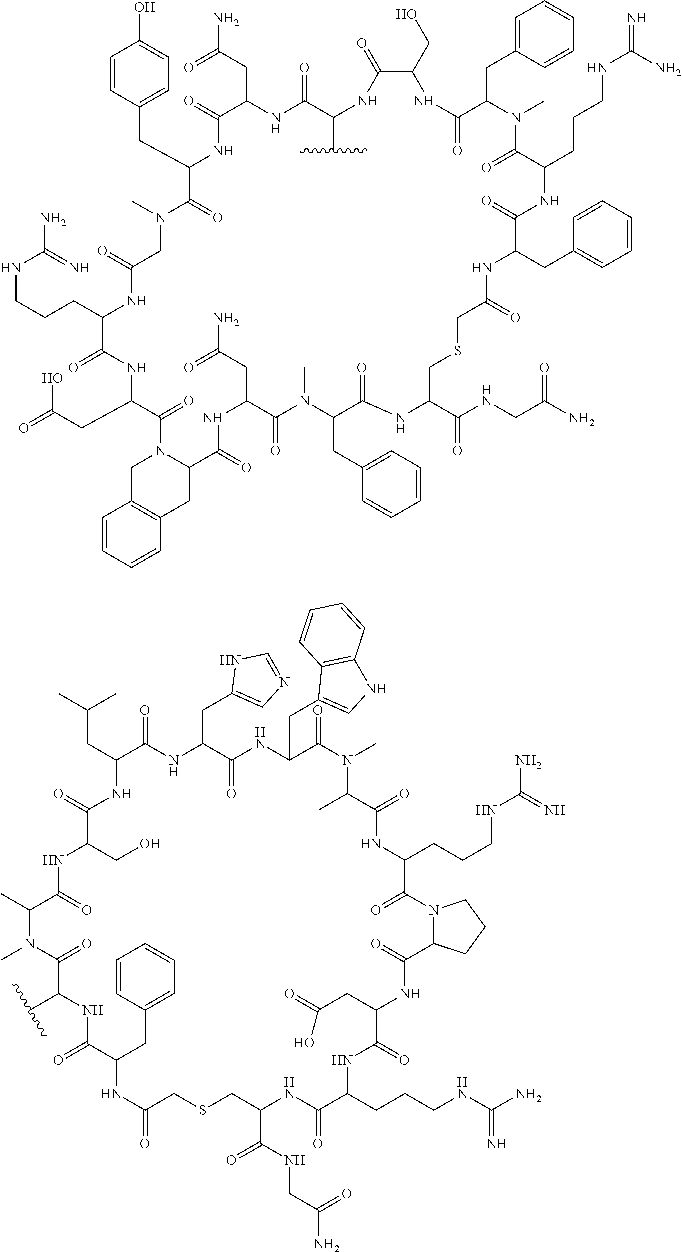 [SMILES: *C1NC(=O)C(CO)NC(=O)C(Cc2ccccc2)N(C)C(=O)C(CCCNC(=N)N)NC(=O)C(Cc2ccccc2)NC(=O)CSCC(C(=O)NCC(N)=O)NC(=O)C(Cc2ccccc2)N(C)C(=O)C(CC(N)=O)NC(=O)C2Cc3ccccc3CN2C(=O)C(CC(=O)O)NC(=O)C(CCCNC(=N)N)NC(=O)CN(C)C(=O)C(Cc2ccc(O)cc2)NC(=O)C(CC(N)=O)NC1=O ; *C1NC(=O)C(Cc2ccccc2)NC(=O)CSCC(C(=O)NCC(N)=O)NC(=O)C(CCCNC(=N)N)NC(=O)C(CC(=O)O)NC(=O)C2CCCN2C(=O)C(CCCNC(=N)N)NC(=O)C(C)N(C)C(=O)C(Cc2c[nH]c3ccccc23)NC(=O)C(Cc2cnc[nH]2)NC(=O)C(CC(C)C)NC(=O)C(CO)NC(=O)C(C)N(C)C1=O]

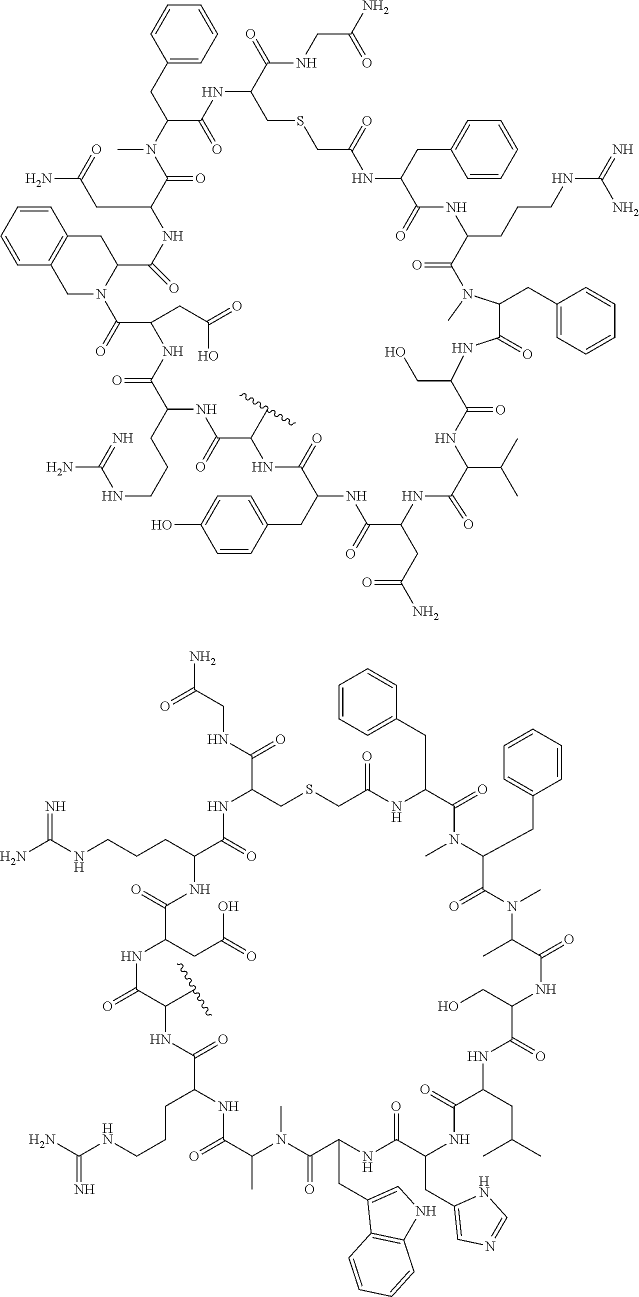 [SMILES: *C1NC(=O)C(Cc2ccc(O)cc2)NC(=O)C(CC(N)=O)NC(=O)C(C(C)C)NC(=O)C(CO)NC(=O)C(Cc2ccccc2)N(C)C(=O)C(CCCNC(=N)N)NC(=O)C(Cc2ccccc2)NC(=O)CSCC(C(=O)NCC(N)=O)NC(=O)C(Cc2ccccc2)N(C)C(=O)C(CC(N)=O)NC(=O)C2Cc3ccccc3CN2C(=O)C(CC(=O)O)NC(=O)C(CCCNC(=N)N)NC1=O ; *C1NC(=O)C(CCCNC(=N)N)NC(=O)C(C)N(C)C(=O)C(Cc2c[nH]c3ccccc23)NC(=O)C(Cc2cnc[nH]2)NC(=O)C(CC(C)C)NC(=O)C(CO)NC(=O)C(C)N(C)C(=O)C(Cc2ccccc2)N(C)C(=O)C(Cc2ccccc2)NC(=O)CSCC(C(=O)NCC(N)=O)NC(=O)C(CCCNC(=N)N)NC(=O)C(CC(=O)O)NC1=O]

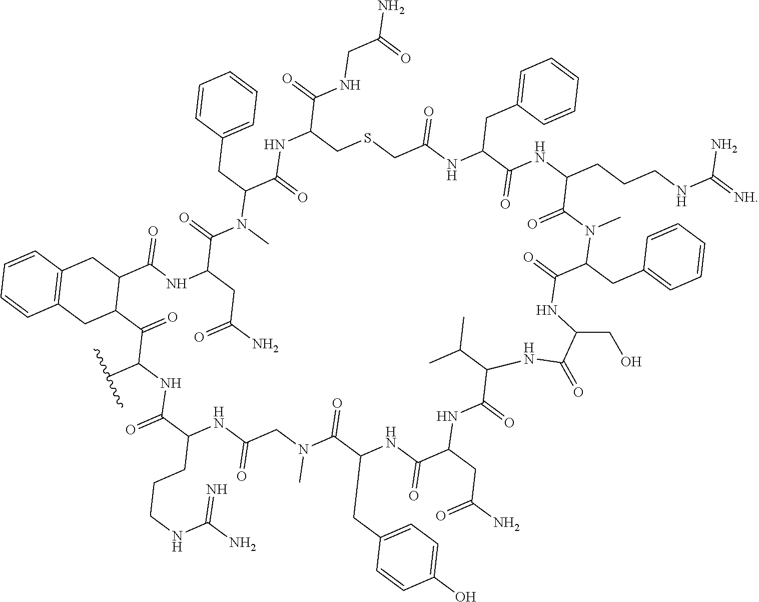 [SMILES: *C1NC(=O)C(CCCNC(=N)N)NC(=O)CN(C)C(=O)C(Cc2ccc(O)cc2)NC(=O)C(CC(N)=O)NC(=O)C(C(C)C)NC(=O)C(CO)NC(=O)C(Cc2ccccc2)N(C)C(=O)C(CCCNC(=N)N)NC(=O)C(Cc2ccccc2)NC(=O)CSCC(C(=O)NCC(N)=O)NC(=O)C(Cc2ccccc2)N(C)C(=O)C(CC(N)=O)NC(=O)C2Cc3ccccc3CC2C1=O]

In certain embodiments, the FHR3 compound is selected from:

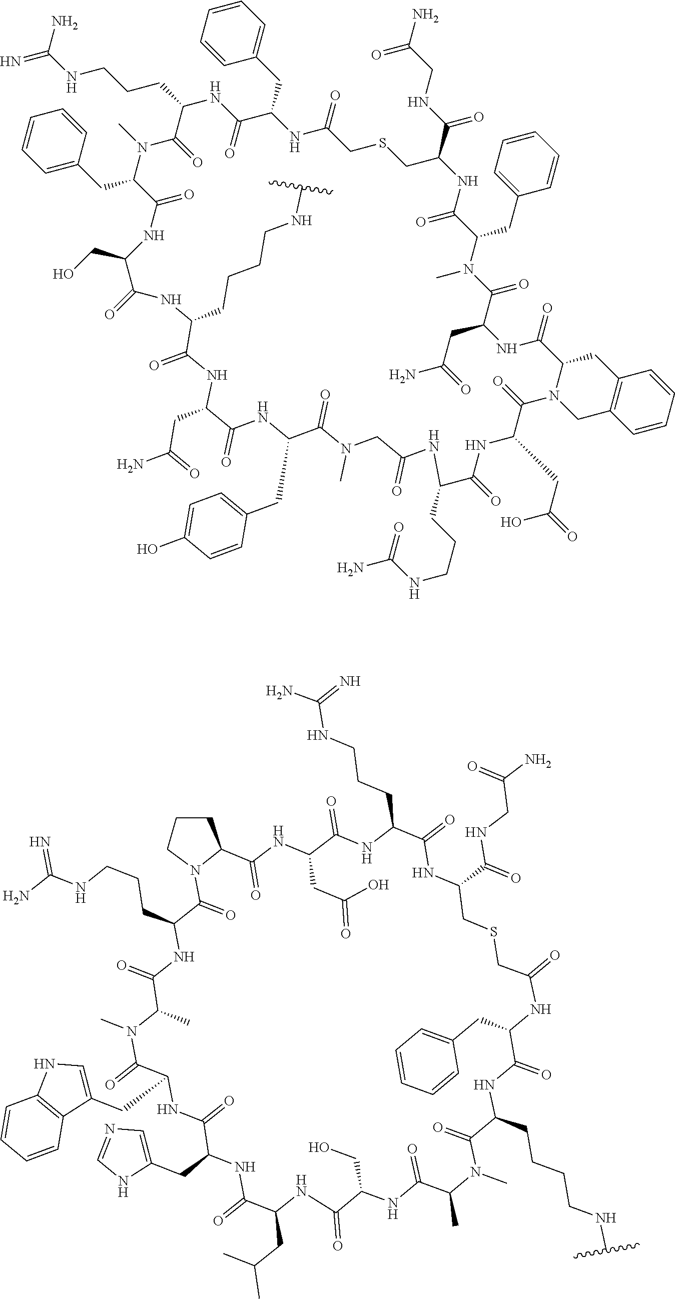

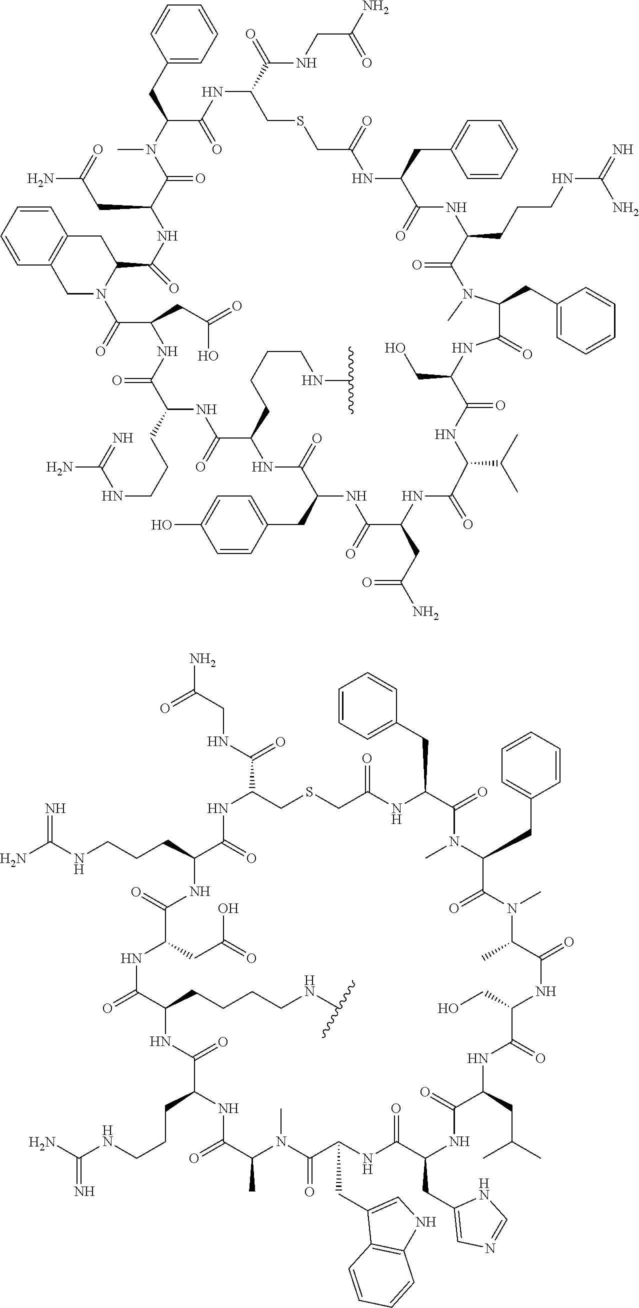

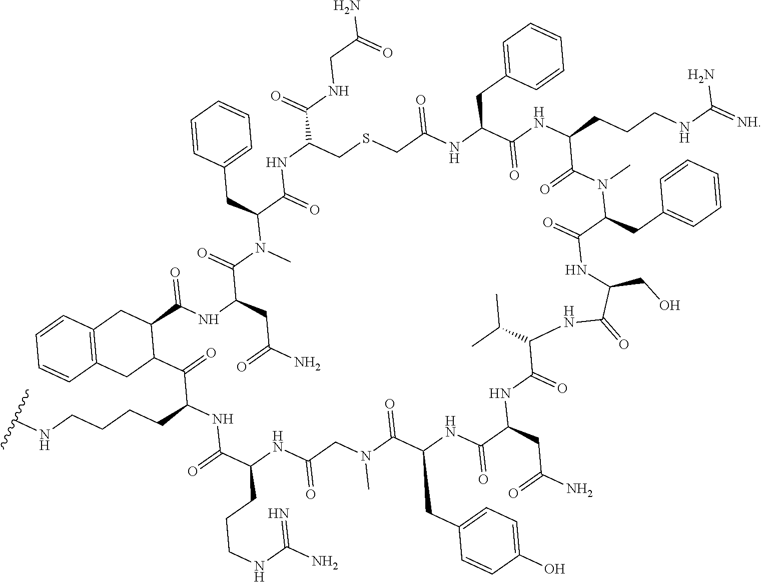

### Tau Protein

In some embodiments, the Target Extracellular Protein is tau protein. The accumulation of tau in the brain causes aggregates that are associated with Alzheimer's and other tauopathies.

Non-limiting examples of Tau Protein targeting ligands include:

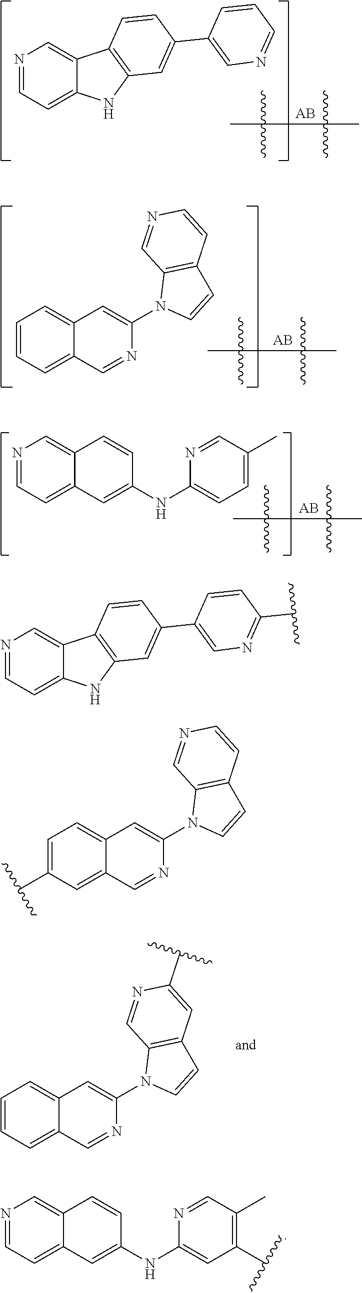

### IL-21

In some embodiments, the Target Extracellular Protein is human interleukin-21 (IL-21) (UniProtKB-Q9HBE4 (IL21_HUMAN)). IL-21 is an immunoregulatory cytokine. IL-21 has been implicated in a number of autoimmune disorders, including Sjogren's syndrome, systemic lupus erythematosus, type 1 diabetes, multiple sclerosis, rheumatoid arthritis, and inflammatory bowel disease.

The Protein Data Bank website provides the crystal structure of IL-21 searchable by 2OQP (Bondensgaard, K., et al., J Biol Chem., 2007, 282 23326-23336); and 4NZD (Hamming et al.); as well as the crystal structure of IL-21 bound to various compounds searchable by 3TGX (Hamming, 0. J., et al., J Biol Chem., 2012, 287(12), 9454-9460).

Representative IL-21 Targeting Ligands are described in FIG. 1. Additional IL-21 Targeting Ligands can be found in, for example, U.S. Pat. No. 9,701,663, which is incorporated herein by reference.

### IL-22

In some embodiments, the Target Extracellular Protein is human interleukin-22 (IL-22) (UniProtKB-Q9GZX6 (IL22 HUMAN)). IL-22 is a member of IL-10 family cytokines that is produced by many different types of lymphocytes including both those of the innate and adaptive immune system. IL-22 has been implicated in a number of autoimmune disorders, including, but not limited to, graft versus host disease (GVHD), psoriasis, rheumatoid arthritis, atopic dermatitis, and asthma.

The Protein Data Bank website provides the crystal structure of IL-22 searchable by 1M4R (Nagem, R. A. P., et al., Structure, 2002, 10 1051-1062); as well as the crystal structure of IL-22 bound to various compounds searchable by 3DGC (Jones, B. C. et al., Structure, 2008, 16 1333-1344).

Representative IL-22 Targeting Ligands are described in FIG. 1. Additional IL-22 Targeting Ligands can be found in, for example, U.S. Pat. No. 9,701,663, which is incorporated herein by reference.

### IL-10

In some embodiments, the Target Extracellular Protein is human interleukin-10 (IL-10) (UniProtKB-P22301 (IL10_HUMAN)). IL-10 is an inflammatory cytokine. IL-10 has been implicated in tumor survival and protection against cytotoxic chemotherapeutic drugs.

The Protein Data Bank website provides the crystal structure of IL-10 searchable by 2TLK (Zdanov, A et al., Protein Sci., 1996, 5 1955-1962); 1ILK (Zdanov, A. et al., Structure, 1995, 3 591-601); 2H24 (Yoon, S. I., et al., J Biol Chem., 2006, 281 35088-35096) and 3LQM (Yoon, S. I., et al., Structure, 2010, 18 638-648). Additionally, Zdanov, A., et al, provides insight into crystal structure of IL-10 (Zdanov A., Current Pharmaceutical design, 2004, 10, 3873-3884).

Representative IL-10 Targeting Ligands are provided in FIG. 1. Additional IL-10 Targeting Ligands can be found, for example, in ACS Chem Biol 11: 2105-11 (2016), which is incorporated herein by reference.

### IL-5

In some embodiments, the Target Extracellular Protein is human interleukin-5 (IL-5) (UniProtKB-P05113 (IL5_HUMAN)). IL-5 is a cytokine that regulates eosinophil maturation, recruitment, and survival. IL-5 has been implicated in a number of allergic disorders, including, but not limited to, asthma, nasal polyposis, atopic dermatitis, eosinophilic esophagitis, hypereosinophilic syndrome, and Churg-Strauss syndrome.

The Protein Data Bank website provides the crystal structure of IL-5 searchable by 1HUL (Milburn, M. V., Nature, 1993, 363, 172-176) and 3VA2 (Kusano et al., Protein Sci., 2012, 21(6), 850-864); as well as the crystal structure of IL-5 bound to various compounds searchable by 1OBX and 1OBZ (Kang, B. S., et al., Structure, 2003, 11, 845).

Representative IL-5 Targeting Ligands are provided in FIG. 1. Additional IL-5 Targeting Ligands can be found, for example, in Bioorg Med Chem 18: 4441-5 (2010); Bioorg Med Chem 18: 4625-9 (2011); Bioorg Med Chem 21: 2543-50 (2013); Eur J Med Chem 59: 31-8 (2013); Bioorg Med Chem 23: 2498-504 (2015); Bioorg Med Chem 20: 5757-62 (2012); each of which is incorporated by reference herein.

### IL8

In some embodiments, the Target Extracellular Protein is human interleukin-8 (IL-8) (UniProtKB-P10145 (IL8_HUMAN)). IL-8 is a chemotactic factor that attracts neutrophils, basophils, and T-cells, but not monocytes. It is also involved in neutrophil activation. It is released from several cell types in response to an inflammatory stimulus. IL-8 has been implicated in the promotion of tumor progression, immune escape, epithelial-mesenchymal transition, and recruitment of myeloid-derived suppressor cells. Studies have demonstrated that high serum IL-8 levels correlate with poor prognosis in many malignant tumors. Preclinical studies have shown that IL-8 blockade may reduce mesenchymal features in tumor cells, making them less resistant to treatment.

The Protein Data Bank website provides the crystal structure of IL-8 searchable by 3IL8 (Baldwin, E. T., et al., Proc Natl Acad Sci USA, 1991, 88, 502-506); and 1IL8 and 2IL8 (Clore, G. M., et al., Biochemistry, 1990, 29, 1689-1696); as well as the crystal structure of IL-8 bound to various compounds searchable by 1ILP and 1ILQ (Skelton, N, J., et al., Structure, 1999, 7, 157-168); and 1ROD (Sticht, H., et al., Eur J Biochem., 1996, 235, 26-35); 4XDX (Ostrov et al.,) and 5WDZ (Beckamp, S., J Biomol NMR, 2017, 69, 111-121).

Representative IL-8 Targeting Ligands are provided in FIG. 1. Additional IL-8 Targeting Ligands can be found in, for example, Bioorg Med Chem Lett 19: 4026-30 (2009), which is incorporated by reference herein.

### Cholinesterase

In some embodiments, the Target Extracellular Protein is human cholinesterase (UniProtKB-P06276 (CHLE_HUMAN)). Cholinesterase contributes to the inactivation of the neurotransmitter acetylcholine. Inhibition of cholinesterase results in increased levels of acetylcholine in the synaptic cleft (the space between two nerve endings). The main use of cholinesterase inhibitors is for the treatment of dementia in patients with Alzheimer's disease. People with Alzheimer's disease have reduced levels of acetylcholine in the brain. Cholinesterase inhibitors have been shown to have an effect on dementia symptoms such as cognition.

The Protein Data Bank website provides the crystal structure of cholinesterase searchable by 1POI and 1POQ (Nicolet, Y., et al., J Biol Chem., 2003, 278, 41141-41147); as well as the crystal structure of cholinesterase bound to various compounds searchable by 1POM and 1POP (Nicolet, Y., et al., J Biol Chem., 2003, 278, 41141-41147); 2J4C (Frasco, M. F., et al., FEBS J., 2007, 274 1849); 4BDT, 4BDS (Nachon, F., et al., Biochem J, 2013, 453, 393-399); 1GQR and 1GQS (Bar-on, P., et al., Biochemistry, 2002, 41, 3555); 3DJY and 3DKK (Carletti, E., et al., J Am Chem Soc., 2008, 130, 16011-16020); 4AXB, 4B00, 4BOP, and 4BBZ (Wandhammer, M., et al., Chem Biol Interact., 2013, 203, 19); 1DX6 (Greenblatt, H. M., et al., FEBS Lett., 1999, 463 321); 1GPK and 1GPN (Dvir, H., et al., Biochemistry, 2002, 41, 10810); 6CQY (Bester, S. M., et al., Chem Res Toxicol., 2018, 31, 1405-1417); 1XLV and 1XLW (Nachon, F., et al., Biochemistry, 2005, 44, 1154-1162); 2Y1K (Carletti, E., et al., Chem Res Toxicol., 2011, 24, 797); and 2WIG, 2WIJ, 2WIK, 2WIL, and 2WSL (Carletti, E., et al., Biochem J., 2009, 421, 97-106). Additionally, Ahmad et al., provides insight into the isolation, crystal structure determination and cholinesterase inhibitory potential of isotalatizidine hydrate from delphinium denudatum (Ahmad H., et al., Journal Pharmaceutical Biology, 2016, 55(1), 680-686).

Representative cholinesterase Targeting Ligands are provided in FIG. 1. Additional Targeting Ligands can be found in, for example, ACS Med Chem Lett 4: 1178-82 (2013); J Med Chem 49: 3421-5 (2006); Eur J Med Chem 55: 23-31 (2012); J Med Chem 51: 3154-70 (2008); J Med Chem 46: 1-4 (2002); Eur J Med Chem 126: 652-668 (2017); Biochemistry 52: 7486-99 (2013); Bioorg Med Chem 23: 1321-40 (2015); which are each incorporated herein by reference.

### C-C Motif Chemokine Ligand 2 (CCL2)

Grygiel et al., provides insight into the synthesis by native chemical ligation and crystal structure of human CCL2 (Grygiel, T. L., et al., Biopolymers, 2010, 94(3), 350-9).

In some embodiments, the Target Extracellular Protein is human C-C motif chemokine ligand 2 (CCL2) (UniProtKB-P13500 (CCL2_HUMAN)). CCL2 acts as a ligand for C-C chemokine receptor CCR2. CCL2 signals through binding and activation of CCR2 and induces a strong chemotactic response and mobilization of intracellular calcium ions. CCL2 exhibits a chemotactic activity for monocytes and basophils but not neutrophils or eosinophils.

CCL2 has been implicated in the recruitment of monocytes into the arterial wall during the disease process of atherosclerosis.

Representative CCL2 Targeting Ligands are provided in FIG. 1. Additional CCL2 Targeting Ligands can be found in, for example, J Med Chem 56: 7706-14 (2013), which is incorporated herein by reference.

### Carboxypeptidase B2

In some embodiments, the Target Extracellular Protein is human carboxypeptidase B2 (UniProtKB-Q96IY4 (CBPB2_HUMAN)). Carboxypeptidase B2, also known as thrombin activatable fibrinolysis inhibitor (TAFIa), cleaves C-terminal arginine or lysine residues from biologically active peptides such as kinins or anaphylatoxins in the circulation thereby regulating their activities. It down-regulates fibrinolysis by removing C-terminal lysine residues from fibrin that has already been partially degraded by plasmin. Carboxypeptidase B2 has been implicated and targeted to inhibit thrombosis.

The Protein Data Bank website provides the crystal structure of carboxypeptidase B2 (also known as thrombin-activatable fibrinolysis inhibitor (TAFI)) searchable by 3D66 (Marx, P. F., et al., Blood, 2008, 112, 2803-2809); 3DGV (Anand, K., et al., JBC, 2008, 283, 29416-29423); and 1KWM (Barbosa Pereira, P. J., et al., J Mol Biol., 2002, 321, 537-547); as well as the crystal structure of TAFI bound to various compounds searchable by 3D67 (Marx, P. F., et al., Blood, 2008, 112, 2803-2809); 5HVF, 5HVG, 5HVH (Zhou, X., et al., J Thromb Haemost., 2016, 14, 1629-1638); and 3LMS (Sanglas, L., et al., J Thromb Haemost., 2010, 8, 1056-1065). Additionally, Schreuder et al., provides insight into the interaction of TAFI and anabaenopeptin, a highly potent inhibitor of TAFI (Schreuder, H., et al., Sci Rep., 2016, 6, 32958).

Representative carboxypeptidase B2 Targeting Ligands are provided in FIG. 1. Additional carboxypeptidase B2 Targeting Ligands can be found in, for example, Bioorg Med Chem Lett 20: 92-6 (2010), J Med Chem 50: 6095-103 (2007), Bioorg Med Chem Lett 14: 2141-5 (2004), J Med Chem 58: 4839-44 (2015), J Med Chem 55: 7696-705 (2012), J Med Chem 59: 9567-9573 (2016), Bioorg Med Chem Lett 17: 1349-54 (2007), U.S. Pat. Nos. 9,662,310, 8,609,710, 9,688,645, J Med Chem 46: 5294-7 (2003), each of which is incorporated herein by reference.

### Neutrophil Elastase

In some embodiments, the Target Extracellular Protein is human neutrophil elastase (UniProtKB-P08246 (ELNE_HUMAN)). Neutrophil elastase modifies the functions of natural killer cells, monocytes and granulocytes. Inhibits C5a-dependent neutrophil enzyme release and chemotaxis.

Neutrophil elastase has been implicated in a number of disorders, including lung disease, chronic obstructive pulmonary disease, pneumonia, respiratory distress, and acute lung injury (ALI), and cystic fibrosis, as well as chronic kidney disease.

The Protein Data Bank website provides the crystal structure of human neutrophil elastase bound to various compounds searchable by 3Q76 and 3Q77 (Hansen, G., et al., J. Mol. Biol., 2011, 409, 681-691); 5ABW (Von Nussbaum, et al., Bioorg Med Chem Lett., 2015, 25, 4370-4381); 1BOF (Cregge, R. J., et al., J Med Chem., 1998, 41, 2461-2480); 1H1B (Macdonald, S. J. F., et al., J Med Chem., 2002, 45, 3878); 2Z7F (Koizumi, M., et al., J Synchrotron Radiat., 2008, 15 308-311); 5A09, 5AOA, 5AOB, and 5AOC (Von Nussbaum, F., et al., Chem Med Chem., 2015, 10, 1163-1173); 5A8X, 5A8Y and 5A8Z (Von Nussbaum, F., et al., Chem Med Chem., 2016, 11, 199-206); 1HNE (Navia, M. A., et al., Proc Natl Acad Sci USA, 1989, 86, 7-11); 6F5M (Hochscherf, J., et al., Acta Crystallogr F Struct Biol Commun., 2018, 74, 480-489); and 4WVP (Lechtenberg, B. C., et al., ACS Chem Biol., 2015, 10, 945-951).

Representative neutrophil elastase Targeting Ligands are provided in FIG. 1. Additional neutrophil elastase Targeting Ligands can be found in, for example, J Med Chem 53: 241-53 (2010), J Med Chem 38: 739-44 (1995), J Med Chem 37: 2623-6 (1994), J Med Chem 38: 4687-92 (1995), J Med Chem 45: 3878-90 (2002), Bioorg Med Chem Lett 5: 105-109 (1995), Bioorg Med Chem Lett 11: 243-6 (2001), J Med Chem 40: 1906-18 (1997), Bioorg Med Chem Lett 25: 4370-81 (2015), U.S. Pat. Nos. 8,569,314, 9,174,997, 9,290,457, each of which is incorporated herein by reference.

### Factor Xa

In some embodiments, the Target Extracellular Protein is human Factor Xa (UniProtKB-P00742 (FA10_HUMAN)). Factor Xa is a vitamin K-dependent glycoprotein that converts prothrombin to thrombin in the presence of factor Va, calcium and phospholipid during blood clotting.

Factor X has been implicated in the development of deep vein thrombosis and acute pulmonary embolism, and the risk of stroke and embolism in people with nonvalvular atrial fibrillation.

The Protein Data Bank website provides the crystal structure of Factor Xa bound to various compounds searchable by 1G2L and 1G2M (Nar, H., et al., Structure, 2001, 9, 29-38); 2PR3 (Nan huis, C. A., et al., Chem Biol Drug Des., 2007, 69, 444-450); 2UWP (Young, R. J., et al., Bioorg Med Chem Lett., 2007, 17, 2927); 2VVC, 2VVV, 2VVU, 2VWL, 2VWM, 2VWN and 2VWO (Zbinden, K. G., et al., Eur J Med Chem., 2009, 44, 2787); 4Y6D, 4Y71, 4Y7A, 4Y7B, 4zh8, 4ZHA (Convery, M. A. et al.); 4Y76, 4Y79, 2J94 and 2J95 (Chan, C., et al., J Med Chem., 2007, 50 1546-1557); 1FAX (Brandstetter, H., et al., J Biol Chem., 1996, 271, 29988-29992); 2JKH (Salonen, L. M., et al., Angew Chem Int Ed Engl., 2009, 48, 811); 2PHB (Kohrt, J. T., et al., Chem Biol Drug Des., 2007, 70, 100-112); 2W26 (Roehrig, S., et al., J Med Chem., 2005, 48, 5900); 2Y5F, 2Y5G and 2Y5H (Salonen, L. M., et al., Chemistry, 2012, 18, 213); 3Q3K (Yoshikawa, K., et al., Bioorg Med Chem Lett., 2011, 21, 2133-2140); 2BMG (Matter, K., et al., J Med Chem., 2005, 48, 3290); 2BOH, 2BQ6 2BQ7, and 2BQW (Nazare, M., et al., J Med Chem., 2005, 48, 4511); 2CJI (Watson, N. S., et al, Bioorg Med Chem Lett., 2006, 16, 3784); 2J2U, 2J34, 2J38, 2J41 (Senger, S., et al., Bioorg Med Chem Lett., 2006, 16 5731); 3IIT (Yoshikawa, K., et al., Bioorg Med Chem., 2009, 17 8221-8233); 1EZQ, 1FOR and 1FOS (Maignan, S., et al., J Med Chem., 2000, 43, 3226-3232); 1FJS (Adler, M., et al., Biochemistry, 2000, 39, 12534-12542); 1KSN (Guertin, K. R., et al., Bioorg Med Chem Lett., 2002, 12, 1671-1674); 1NFU, 1NFW, 1NFX and 1NFY (Maignan, S., et al., J Med Chem., 2003, 46, 685-690); 2XBV, 2XBW, 2XBX, 2XBY, 2XCO, 2XC4 and 2XC5 (Anselm, L., et al., Bioorg Med Chem Lett., 2010, 20, 5313); 4A7I (Nazare, M., et al., Angew Chem Int Ed Engl., 2012, 51, 905); 4BTI, 4BTT and 4BTU (Meneyrol, L., et al., J Med Chem., 2013, 56, 9441); 3FFG, 3KQB, 3KQC, 3KQD and 3KQE (Quan, M. L., et al., Bioorg Med Chem Lett., 2010, 20, 1373-1377); 2P93, 2P94 and 2P95 (Qiao, J. X., et al., Bioorg Med Chem Lett., 2007, 17, 4419-4427); 1V3X (Haginoya, N., et al., J Med Chem., 2004, 47, 5167-5182); 2P16 (Pinto, D. J. P., et al., J Med Chem., 2007, 50, 5339-5356); 2RAO (Lee, Y. K., et al., J Med Chem., 2008, 51, 282-297); 3SW2 (Shi, Y., et al., Bioorg Med Chem Lett., 2011, 21, 7516-7521); 2VH6 (Young, R. J., et al., Bioorg Med Chem Lett., 2008, 18, 23); 2WYG and 2WYJ (Kleanthous, S., et al., Bioorg Med Chem Lett., 2010, 20, 618); 2Y7X (Watson, N. S., et al., Bioorg Med Chem Lett., 2011, 21, 1588); 2Y7Z, 2Y80, 2Y81 and 2Y82 (Young, R. J., et al., Bioorg Med Chem Lett., 2011, 21, 1582); 3KL6 (Fujimoto, T., et al., J Med Chem., 2010, 53, 3517-3531); 3LIW (Meuller, M. M., et al., Biol. Chem., 2003, 383, 1185); 5KOH (Schweinitz, A., et al., Med Chem., 2006,2, 349-361); 1XKA and 1XKB (Kamata, K., et al., Proc Natl Acad Sci USA, 1998, 95, 6630-6635); 2EI6 and 2EI7 (Nagata, T., et al., Bioorg Med Chem Lett., 2007, 17, 4683-4688); 2P3T (Ye, B., et al., J Med Chem., 2007, 50, 2967-2980); 1MQ5 and 1MQ6 (Adler, M., et al., Biochemistry, 2002, 41, 15514-15523); 3K9X and 3HPT (Shi, Y., et al., Bioorg Med Chem Lett., 2009, 19, 6882-6889); 3CEN (Corte, J. R., et al., Bioorg Med Chem Lett., 2008, 18, 2845-2849); 2W3I and 2W3K (Van Huis, C. A., et al., Bioorg Med Chem., 2009, 17, 2501); 2H9E (Murakami, M. T., et al., J Mol Biol., 2007, 366, 602-610); 1WU1 and 2D1J (Komoriya, S., et al., Bioorg Med Chem., 2005, 13, 3927-3954); 2G00 (Pinto, D. J. P., et al., Bioorg Med Chem Lett., 2006, 16, 5584-5589); 3M36 and 3M37 (Pruitt, J. R. et al., J Med Chem., 2003, 46, 5298-5315); 3CS7 (Qiao, J. X., et al., Bioorg Med Chem Lett., 2008, 18, 4118-4123); 1Z6E (Quan, M. L., et al., J Med Chem., 2005, 48, 1729-1744); 2FZZ (Pinto, D. J. P., et al., Bioorg Med Chem Lett., 2006, 16, 4141-4147); and 3ENS (Shi, Y., et al., J Med Chem., 2008, 51, 7541-7551).

Representative Factor Xa Targeting Ligands are provided in FIG. 1. Additional Factor Xa Targeting Ligands can be found in, for example, Bioorg Med Chem Lett 20: 5313-9 (2010), Bioorg Med Chem Lett 13: 679-83 (2003), J Med Chem 44: 566-78 (2001), J Med Chem 50: 2967-80 (2007), J Med Chem 38: 1511-22 (1995), Bioorg Med Chem Lett 18: 2845-9 (2008), J Med Chem 53: 6243-74 (2010), Bioorg Med Chem Lett 18: 2845-9 (2008), Bioorg Med Chem 16: 1562-95 (2008), each of which is incorporated herein by reference.

### Factor XI

In some embodiments, the Target Extracellular Protein is human Factor XI UniProtKB-P03951 (FA11_HUMAN). Factor XI triggers the middle phase of the intrinsic pathway of blood coagulation by activating factor IX.

Factor XI has been implicated in the development of deep vein thrombosis and acute pulmonary embolism, and the risk of stroke and embolism in people with nonvalvular atrial fibrillation.

The Protein Data Bank website provides the crystal structure of Factor XI bound to various compounds searchable by 1ZSL, 1ZTJ, 1ZTK, and 1ZTL (Nagafuji, P., et al.,); 1ZOM (Lin, J., et al., J Med Chem., 2006, 49, 7781-7791); 5EOK and 5EOD (Wong, S. S., et al., Blood, 2016, 127, 2915-2923); 1ZHM, 1ZHP and 1ZHR (Jin, L., et al., Acta Crystallogr D Biol Crystallogr., 2005, 61, 1418-1425); 1ZMJ, 1ZLR, 1ZML and 1ZMN (Lazarova, T. I., Bioorg Med Chem Lett., 2006, 16, 5022-5027); 1ZRK, 1ZSJ and 1ZSK (Guo, Z., et al); 4CRA, 4CRB, 4CRC, 4CRD, 4CRE, 4CRF and 4CRG (Fjellstrom, O., et al., PLoS One, 2015, 10, 13705); 3SOR and 3SOS (Fradera, X., et al., Acta Crystallogr Sect F Struct Biol Cryst Commun., 2012, 68, 404-408); 1ZPB, 1ZPC, 2FDA (Deng, H., et. al., Bioorg Med Chem Lett., 2006, 16, 3049-3054); 5WB6 (Wang, C., et al., Bioorg Med Chem Lett., 2017, 27, 4056-4060); 4NA7 and 4NA8 (Quan, M. L., et al., J Med Chem., 2014, 57, 955-969); 4WXI (Corte, J. R., et al., Bioorg Med Chem Lett., 2015, 25, 925-930); 5QTV, 5QTW, 5QTX and 5QTY (Fang, T., et al., Bioorg Med Chem Lett., 2020, 126949-126949); 6COS (Hu, Z., et al., Bioorg Med Chem Lett., 28, 987-992); 5QQP and 5QQO (Clark, C. G., et al., Bioorg Med Chem Lett., 2019, 29, 126604-126604); 5QOD, 5QOE, 5QOF, 5QOG, and 5QOH (Corte, J. R., et al., Bioorg Med Chem Lett., 2017, 27, 3833-3839); 5QCK, 5QCL, 5QCM, and 5QCN (Pinto, D. J. P., et al., J Med Chem., 2017, 60, 9703-9723); 5TKS and 5TKU (Corte, J. R., et al., J Med Chem., 2017, 60, 1060-1075); 1XXD and 1XX9 (Jin, L., et al., J Biol Chem., 2005, 280, 4704-4712); 5QTT and 5QTU (Corte, J. R., et al., J Med Chem., 2019, 63, 784-803); 4TY6, 4TY7 (Hangeland, J. J., et al., J Med Chem., 2014, 57, 9915-9932); 4X6M, 4X6N, 4X60, and 4X6P (Pinto, D. J. P., et al., Bioorg Med Chem Lett., 2015, 25, 1635-1642); and 5EXM (Corte, J. R., et al., Bioorg Med Chem., 2016, 24, 2257-2272). Additionally, Al-Horani et al., provides insight into a review of patent literature regarding Factor Xia inhibitors (Al-Horani et al., Expert Opin Ther Pat. 2016; 26(3), 323-345).

Representative Factor XI Targeting Ligands are provided in FIG. 1. Additional Factor XI Targeting Ligands can be found in, for example, U.S. Pat. No. 9,783,530, 10/143,681, 10/214,512, ACS Med Chem Lett 6: 590-5 (2015), J Med Chem 60: 9703-9723 (2017), J Med Chem 60: 9703-9723 (2017), U.S. Pat. No. 9,453,018 (2016), J Med Chem 60: 1060-1075 (2017), J Med Chem 57: 955-69 (2014), each of which is incorporated herein by reference.

In certain embodiments the Factor XI Targeting Ligand is selected from:

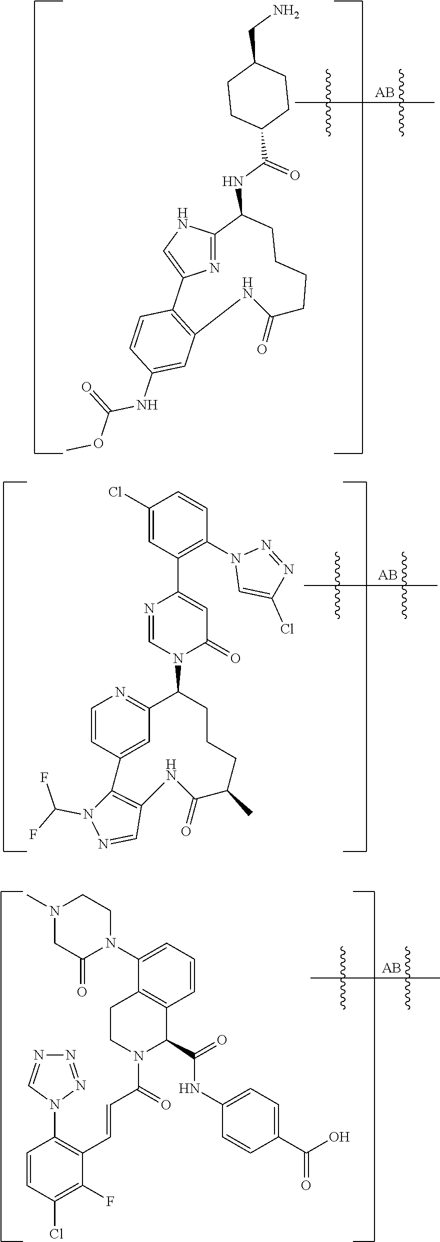

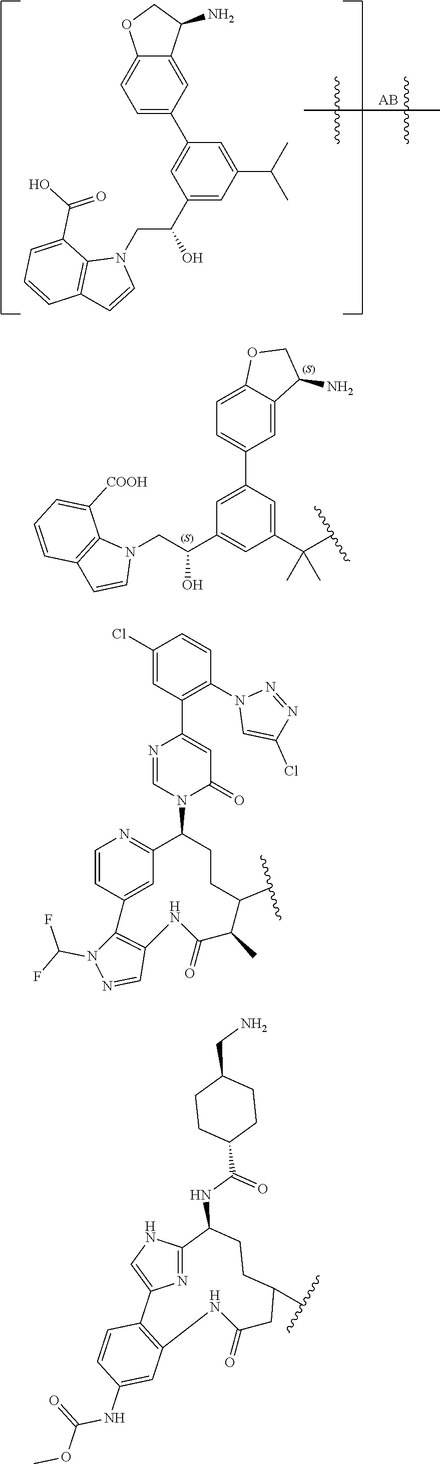

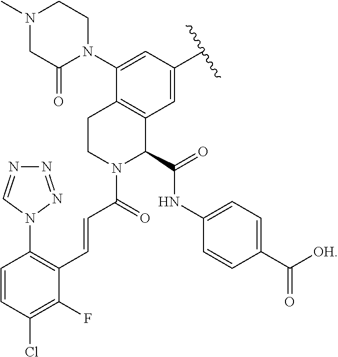 [SMILES: *c1cc2c(c(N3CCN(C)CC3=O)c1)CCN(C(=O)/C=C/c1c(-n3cnnn3)ccc(Cl)c1F)[C@@H]2C(=O)Nc1ccc(C(=O)O)cc1]

In certain embodiments the Factor XI Targeting Ligand is described in J Med Chem 61 (17), 7425-7447 (2018) or J Med Chem (2020) Structure-based design and pre-clinical characterization of selective and orally bioavailable Factor Xia inhibitors: demonstrating the power of an integrated Si protease family approach.

### Factor XII

In some embodiments, the Target Extracellular Protein is human Factor XII (UniProtKB-P00748 (FA12_HUMAN)). Factor XII is a serum glycoprotein that participates in the initiation of blood coagulation, fibrinolysis, and the generation of bradykinin and angiotensin. Prekallikrein is cleaved by factor XII to form kallikrein, which then cleaves factor XII first to alpha-factor XIIa and then trypsin cleaves it to beta-factor XIIa. Alpha-factor XIIa activates factor XI to factor XIa.

Factor XII has been implicated in the development of deep vein thrombosis and acute pulmonary embolism, and the risk of stroke and embolism in people with nonvalvular atrial fibrillation.

The Protein Data Bank website provides the crystal structure of factor XII bound to various compounds searchable by 4XDE and 4XE4 (Pathak, M., et al., J Thromb Haemost., 2015, 13(4), 580-591); 6GT6 and 6QF7 (Pathak, M., et al., Acta Crystallogr D Struct Biol., 2019, 75, 578-591); and 6B74 and 6B77 (Dementiev, A. A., et al., Blood Adv., 2018, 2, 549-558). Additionally, Pathak et al., provides insight into the crystal structure of factor XII (Pathak, M., et al., J Thromb Haemost., 2015, 13(4), 580-591).

Representative Factor XII Targeting Ligands are provided in FIG. 1. Additional Factor XII Targeting Ligands can be found in, for example, J Med Chem 60: 1151-1158 (2017), J Med Chem 48: 2906-15 (2005), J Med Chem 50: 5727-34 (2007), J Med Chem 50: 1876-85 (2007), Chembiochem 18: 387-395 (2017), each of which is incorporated herein by reference.

### Factor XIII

In some embodiments, the Target Extracellular Protein is human Factor XIII UniProtKB-P00488 (F13A_HUMAN)). Factor XIII is activated by thrombin and calcium ion to a transglutaminase that catalyzes the formation of gamma-glutamyl-epsilon-lysine cross-links between fibrin chains, thus stabilizing the fibrin clot. Also cross-link alpha-2-plasmin inhibitor, or fibronectin, to the alpha chains of fibrin.

Factor XIII has been implicated in the development of deep vein thrombosis and acute pulmonary embolism, and the risk of stroke and embolism in people with nonvalvular atrial fibrillation.

The Protein Data Bank website provides the crystal structure of factor XIII searchable by 1FIE (Yee, V. C., et al., Thromb Res., 1995, 78, 389-397); and 1F13 (Weiss, M. S., et al., FEBS Lett., 1998, 423, 291-296); as well as the crystal structure of factor XIII bound to various compounds searchable by 1DE7 (Sadasivan, C., et al., J Biol Chem., 2000, 275, 36942-36948); and 5MHL, 5MHM, 5MHN, and 5MHO (Stieler, M., et al.,). Additionally, Gupta et al., provides insight into the mechanism of coagulation factor XIII activation and regulation from a structure/functional perspective (Gupta, S., et al., Sci Rep., 2016; 6, 30105); and Komaromi et al., provides insight into the novel structural and functional aspect of factor XIII (Komaromi, Z., et al., . J Thromb Haemost 2011, 9, 9-20).

Representative Factor XIII Targeting Ligands are provided in FIG. 1. Additional Factor XIII Targeting Ligands can be found in, for example, Eur J Med Chem 98: 49-53 (2015), J Med Chem 55: 1021-46 (2012), J Med Chem 48: 2266-9 (2005), each of which is incorporated herein by reference.

### Prothrombin

In some embodiments, the Target Extracellular Protein is human Prothrombin (UniProtKB-P00734 (THRB_HUMAN)). Thrombin, which cleaves bonds after Arg and Lys, converts fibrinogen to fibrin and activates factors V, VII, VIII, XIII, and, in complex with thrombomodulin, protein C. Functions in blood homeostasis, inflammation and wound healing.

Thrombin is involved in blood clot formation and arterial and venous thrombosis, and thromboembolism associated with atrial fibrillation.

The Protein Data Bank website provides the crystal structure of prothrombin searchable by 3NXP (Chen, Z. et al., Proc Natl Acad Sci USA, 2010, 107, 19278-19283); as well as the crystal structure of prothrombin bound to various compounds searchable by 2HPP and 2IPQ (Arni, R. K., et al., Biochemistry, 1993, 32, 4727-4737); 6BJR, 6C2W (Chinnaraj, M., et al., Sci Rep., 2018, 8, 2945-2945); 5EDK, 5EDM (Pozzi, N., et al., J Biol Chem., 2016, 291, 6071-6082); 3K65 (Adams, T. E., et al., Biochimie, 2016, 122, 235-242); and 6BJR and 6C2W (Chinnaraj, M. et al., Sci Rep., 2018, 8, 2945-2945). Additionally, Pozzi et al., provides insight into the mechanism and conformational flexibility for the crystal structure of prothrombin (Pozzi, N. et al., J Biol Chem., 2013, 288(31), 22734-22744); and Zhiwei et al., provides insight into the crystal structure of prothrombin-1 (Zhiwei, C. et al., PNAS, 2010, 107(45), 19278-19283).

Prothrombin is converted to thrombin, as such the Protein Data Bank website provides the crystal structure of thrombin bound to compounds searchable by 1XMN (Carter, W. J. et al., J. Biol. Chem., 2005, 280, 2745-2749); 4CH₂ and 4CH₈ (Lechtenberg, B. C. et al., J Mol Biol., 2014, 426, 881); 3P01 (Karle, M. et al., Bioorg Med Chem Lett., 2012, 22, 4839-4843); 3DA9 (Nilsson, M. et al., J Med Chem., 2009, 52, 2708-2715); 2H9T and 3BF6 (Lima, L. M. T. R. et al., Biochim Biophys Acta., 2009, 1794, 873-881); 3BEF and 3BEI (Gandhi, P. S. et al., Proc Natl Acad Sci USA, 2008, 105, 1832-1837); 3BV9 (Nieman, M. T. et al., J Thromb Haemost., 2008, 6, 837-845); 2HWL (Pineda, A. O. et al., Biophys Chem., 2007, 125, 556-559); 2AFQ (Johnson, D. J. D. et al., Biochem J., 2005, 392, 21-28); 1SHH (Pineda, A. O. et al., J Biol Chem., 2004, 279, 31842-31853); 1JWT (Levesque, S. et al., Bioorg Med Chem Lett., 2001, 11, 3161-3164); 1G37 (Bachand, B. et al., Bioorg Med Chem Lett., 2001, 11, 287-290); 1EOJ and 1EOL (Slon-Usakiewicz, J. J. et al., Biochemistry, 2000, 39, 2384-2391); 1AWH (Weir, M. P. et al., Biochemistry, 1998, 37, 6645-6657); 1DIT (Krishnan, R. et al., Protein Sci., 1996, 5, 422-433); 1HAO and 1HAP (Padmanabhan, K. et al., Acta Crystallogr D Biol Crystallogr., 1996, 52, 272-282); and 1HBT (Rehse, P. H. et al., Biochemistry, 1995, 34, 11537-11544).

Representative prothrombin Targeting Ligands are provided in FIG. 1. Additional prothrombin Targeting Ligands can be found in, for example, J Med Chem 46: 3612-22 (2003), Bioorg Med Chem Lett 12: 1017-22 (2002), J Med Chem 40: 830-2 (1997), Bioorg Med Chem Lett 15: 2771-5 (2005), J Med Chem 42: 3109-15 (1999), J Med Chem 47: 2995-3008 (2004), Bioorg Med Chem 16: 1562-95 (2008), J Med Chem 42: 3109-15 (1999), each of which is incorporated herein by reference.

### Coagulation Factor VII

In some embodiments, the Target Extracellular Protein is human coagulation Factor VII (UniProtKB-P08709 (FA7_HUMAN)). Factor VII initiates the extrinsic pathway of blood coagulation. It is a serine protease that circulates in the blood in a zymogen form. Factor VII is converted to Factor VIIa by Factor Xa, Factor XIIa, Factor IXa, or thrombin by minor proteolysis.

In the presence of tissue factor and calcium ions, Factor VIIa then converts Factor X to Factor Xa by limited proteolysis. Factor VIIa will also convert Factor IX to Factor IXa in the presence of tissue factor and calcium.

Factor VII is involved in blood clot formation and arterial and venous thrombosis, and thromboembolism associated with atrial fibrillation.

The Protein Data Bank website provides the crystal structure of factor VII bound to various compounds searchable by 2F9B (Rai, R., et al., Bioorg Med Chem Lett., 2006, 16, 2270-2273); 5U6J (Wurtz, N. R., et al., Bioorg Med Chem Lett., 2017, 27, 2650-2654); 5L2Y, 5L2Z, and 5L30 (Ladziata, .U., et al., Bioorg Med Chem Lett., 2016, 26, 5051-5057); 5146 (Glunz, P. W., et al., J Med Chem., 2016, 59, 4007-4018); 4YLQ, 4Z6A, and 4ZMA (Sorensen, A. B., et al., J Biol Chem., 2016, 291, 4671-4683); 4YT6 and 4YT7 (Glunz, P. W., et al., Bioorg Med Chem Lett, 2015, 25, 2169-2173); 4NA9 (Quan, M. L., et al., J Med Chem., 2014, 57, 955-969); 4NG9 (hang, X., et al., ACS Med Chem Lett., 2014, 5, 188-192); 4JZD, 4JZE and 4JZF (Bolton, S. A., et al., Bioorg Med Chem Lett., 2013, 23, 5239-5243); 4JYU and 4JYV (Glunz, P. W., et al., Bioorg Med Chem Lett., 2013, 23, 5244-5248); 4ISH (Priestley, E. S., et al., Bioorg Med Chem Lett., 2013, 23, 2432-2435); 4ISI (Zhang, X., et al., Bioorg Med Chem Lett., 2013, 23, 1604-1607); 2ZZU (Shiraishi, T., et al., Chem Pharm Bull (Tokyo), 2010, 58, 38-44); 1WV7 and 1WUN (Kadono, S., et al., Biochem Biophys Res Commun., 2005, 327, 589-596); 2ZWL, 2ZPO, (Kadono, S., et al.); 2EC9 (Krishan, R., et al., Acta Crystallogr D Biol Crystallogr., 2007, 63, 689-697); 2PUQ (Larsen, K. S., et al., Biochem J., 2007, 405, 429-438); 2FLR (Riggs, J. R., et al., Bioorg Med Chem Lett., 2006, 16, 3197-3200); 2C4F (Kohrt, J. T., et al., Bioorg Med Chem Lett., 2006, 16, 1060); 2AEI (Kohrt, J. T. et al., Bioorg Med Chem Lett., 2005, 15, 4752-4756); 1WTG (Kadono, S., et al., Biochem Biophys Res Commun., 2005, 326, 859-865); 1WSS (Kadono, S., et al., Acta Crystallogr SectF Struct Biol Cryst Commun., 2005, 61, 169-173); 1W7X and 1W8B (Zbinden, K. G., et al., Bioorg Med Chem Lett., 2005, 15, 5344); 1WQV (Kadono, S., et al., Biochem Biophys Res Commun., 2004, 324, 1227-1233); 1Z6J (Schweitzer, B. A., et al., Bioorg Med Chem Lett., 2005, 15, 3006-3011); 1YGC (Olivero, A. G., et al., J Biol Chem., 2005, 280, 9160-9169); 6R2W (Sorensen, A. B., et al., J Biol Chem., 2019, 295, 517-528); 5PA8, 5PA9, 5PAA, 5PAB, 5PAC, 5PAE, 5PAF, 5PAG, 5PAI, 5PAJ, 5PAK, 5PAM, 5PAN, 5PAO, 5PAQ, 5PAR, 5PAS, 5PAT, 5PAU, 5PABV, 5PAW, 5PAX, 5PAY, 5PBO, 5PB1, 5PB2, 5PB3, 5PB4, 5PB5, and 5PB6 (Mayweg, A. V., et al.,); and 5LOS (Li, Z., et al., Nat Commun., 2017, 8, 185-185). Additionally, Kemball-Cook, et al., provides insight into the crystal structure of active site-inhibited factor VIIa (Kemball-Cook, G., et al., J Struct Biol., 1999, 127(3), 213-23).

Representative Factor VII Targeting Ligands are provided in FIG. 1. Additional Factor VII Targeting Ligands can be found in, for example, U.S. Pat. No. 9,174,974, Bioorg Med Chem Lett 26: 5051-5057 (2016), Bioorg Med Chem Lett 11: 2253-6 (2001), Bioorg Med Chem Lett 15: 3006-11 (2005), Bioorg Med Chem Lett 12: 2883-6 (2002), each of which is incorporated herein by reference.

### Coagulation Factor IX

In some embodiments, the Target Extracellular Protein is human coagulation Factor IX (UniProtKB-P00740 (FA9_HUMAN)). Factor IX Factor IX is a vitamin K-dependent plasma protein that participates in the intrinsic pathway of blood coagulation by converting factor X to its active form in the presence of Ca2+ ions, phospholipids, and factor VIIIa.

Factor IX is involved in blood clot formation and arterial and venous thrombosis, and thromboembolism associated with atrial fibrillation.

The Protein Data Bank website provides the crystal structure of factor IX bound to various compounds searchable by 6MV4 (Vadivel, K., et al., J Thromb Haemost., 2019, 17, 574-584); 4ZAE (Zhang, T., et al., Bioorg Med Chem Lett., 2015, 25, 4945-4949); 4YZU and 4ZOK (Parker, D. L., et al., Bioorg Med Chem Lett., 2015, 25, 2321-2325); 5TNO and 5TNT (Sakurada, I., et al., Bioorg Med Chem Lett., 2017, 27, 2622-2628); 5JB8, 5JB9, 5JBA, 5JBB and 5JBC (Kristensen, L. H., et al., Biochem J., 2016, 473, 2395-2411); 3LC3 (Wang, S., et al., J Med Chem., 2010, 53, 1465-1472); 3LC5 (Wang, S., et al., J Med Chem., 2010, 53, 1473-1482); 3KCG (Johnson, D. J. D., et al., Proc Natl Acad Sci USA, 2010, 107, 645-650); 1NLO (Huang, M., et al., J Biol Chem., 2004, 279, 14338-14346); 1RFN (Hopfner, K. P., et al., Structure, 1999, 7, 989-996); and 6RFK (Sendall, T. J., et al.,).

Representative Factor IX Targeting Ligands are provided in FIG. 1. Additional Factor IX Targeting Ligands can be found in, for example, U.S. Pat. No. 9,409,908, Bioorg Med Chem Lett 25: 5437-43 (2015), U.S. patent Ser. No. 10/189,819, each of which is incorporated herein by reference.

### Fibroblast Growth Factor 1 (FGF1)

In some embodiments, the Target Extracellular Protein is human fibroblast growth factor 1 (FGF1) (UniProtKB-P05230 (FGF1HUMAN)). FGF1 plays an important role in the regulation of cell survival, cell division, angiogenesis, cell differentiation and cell migration. FGF1 acts as a ligand for FGFR1 and integrins, and binds to FGFR1 in the presence of heparin leading to FGFR1 dimerization and activation via sequential autophosphorylation on tyrosine residues which act as docking sites for interacting proteins, leading to the activation of several signaling cascades. FGF1 induces the phosphorylation and activation of FGFR1, FRS2, MAPK3/ERK1, MAPK1/ERK2 and AKT1. FGF1 can induce angiogenesis. FGF1 has been implicated in oncogenesis, cancer cell proliferation, resistance to anticancer therapies, and neoangiogenesis.

The Protein Data Bank website provides the crystal structure of FGF1 searchable by 2AFG (Blaber, M., et al., Biochemistry, 1996, 35, 2086-2094); and 1BAR (Zhu, X. et al., Science, 1991, 251, 90-93); as well as the crystal structure of FGF1 bound to various compounds searchable by 1AFC (Zhu, X., et al., Structure, 1993, 1, 27-34); 1AXM and 2AXM (DiGabriele, A. D., et al., Nature, 1998, 393, 812-817); IEVT (Plotnikov, A. N., et al., Cell, 2000, 101, 413-424); 1E0O (Pellegrini, L., et al., Nature, 2000, 407, 1029); and 2ERM (Canales, A., et al., FEBS J, 2006, 273, 4716-4727).

Representative FGF1 Targeting Ligands are provided in FIG. 1. Additional FGF1 Targeting Ligands can be found in, for example, Bioorg Med Chem Lett 18: 344-9 (2008), Chembiochem 6: 1882-90 (2005), J Med Chem 55: 3804-13 (2012), J Med Chem 47: 1683-93 (2004), J Med Chem 53: 1686-99 (2010,) each of which is incorporated herein by reference.

### Fibroblast Growth Factor 2 (FGF2)

In some embodiments, the Target Extracellular Protein is human fibroblast growth factor 2 (FGF2) (UniProtKB-P09038 (FGF2_HUMAN)). FGF2 acts as a ligand for FGFR1, FGFR2, FGFR3 and FGFR4. FGF2 also acts as an integrin ligand which is required for FGF2 signaling, and plays an important role in the regulation of cell survival, cell division, cell differentiation and cell migration. FGF2 also induces angiogenesis. FGF2 has been implicated in oncogenesis, cancer cell proliferation, resistance to anticancer therapies, and neoangiogenesis.

The Protein Data Bank website provides the crystal structure of FGF2 bound to various compounds searchable by 40EE, 40EF, and 40EG (Li, Y. C., et al., ACS Chem Biol., 2014, 9, 1712-1717); 1EV2 (Plotnikov, A. N., et al., Cell, 2000, 101, 413-424); and 5X1O (Tsao, Y. H.).

Representative FGF2 Targeting Ligands are provided in FIG. 1. Additional FGF2 Targeting Ligands can be found in, for example, U.S. Pat. No. 8,933,099, Bioorg Med Chem Lett 12: 3287-90 (2002), Chem Biol Drug Des 86: 1323-9 (2015), Bioorg Med Chem Lett 25: 1552-5 (2015), each of which is incorporated herein by reference.

### Fibronectin-1

In some embodiments, the Target Extracellular Protein is human fibronectin 1 (FN1) (UniProtKB-P02751 (FINC_HUMAN)). Fibronectin (FN) polymerization is necessary for collagen matrix deposition and is a key contributor to increased abundance of cardiac myofibroblasts (MFs) after cardiac injury. Interfering with FN polymerization may attenuate MF and fibrosis and improve cardiac function after ischemia/reperfusion (I/R) injury.

The Protein Data Bank website provides the crystal structure of fibronectin-1 bound to various compounds searchable by 3M7P (Graille, M., et al., Structure, 2010, 18, 710-718); 3MQL (Erat, M. C., et al., J Biol Chem., 2010, 285, 33764-33770); and 3EJH (Erat, M. C., et al., Proc Natl Acad Sci USA, 2009, 106, 4195-4200).

Representative FN Targeting Ligands are provided in FIG. 1. Additional FN Targeting Ligands can be found in, for example, Bioorg Med Chem Lett 18: 2499-504 (2008), which is incorporated herein by reference.

### Kallikrein-1 (KLK1)

In some embodiments, the Target Extracellular Protein is human kallikrein-1 (UniProtKB-P06870 (KLK1_HUMAN)). Glandular kallikreins cleave Met-Lys and Arg-Ser bonds in kininogen to release Lys-bradykinin. Kallikrein has been implicated in adverse reactions in hereditary angioedema (HAE).

The Protein Data Bank website provides the crystal structure of KLK1 searchable by 1SPJ (Laxmikanthan, G., et al., Proteins, 2005, 58, 802-814); as well as the crystal structure of KLK1 bound to various compounds searchable by 5F8Z, 5F8T, 5F8X, (Xu, M., et al.,); and 6A80 (Xu, M., et al., FEBS Lett., 2018, 592, 2658-2667). Additionally, Katz et al., provides insight into the crystal structure of kallikrein (Katz, B. A., et al., Protein Sci., 1998, 7(4), 875-85).

Representative kallikrein Targeting Ligands are provided in FIG. 1. Additional kallikrein Targeting Ligands can be found in, for example, U.S. Pat. No. 9,783,530, J Med Chem 38: 2521-3 (1995), U.S. Pat. No. 9,234,000, 10/221,161, U.S. Pat. Nos. 9,687,479, 9,670,157, 9,834,513, J Med Chem 38: 1511-22 (1995), U.S. patent Ser. No. 10/214,512, each of which is incorporated herein by reference.

### Plasma Kallikrein

In some embodiments, the Target Extracellular Protein is human plasma kallikrein (UniProtKB-P03952 (KLKB1_HUMAN)). Plasma kallikrein cleaves Lys-Arg and Arg-Ser bonds. It activates, in a reciprocal reaction, factor XII after its binding to a negatively charged surface. It also releases bradykinin from HMW kininogen and may also play a role in the renin-angiotensin system by converting prorenin into renin. Plasma kallikrein has been implicated in retinal dysfunction, the development of diabetic macular edema and hereditary angioedema (HAE).

The Protein Data Bank website provides the crystal structure of plasma kallikrein bound to various compounds searchable by 5TJX (Li, Z., et al., ACS Med Chem Lett., 2017, 8, 185-190); 601G and 601S (Patridge, J. R., et al., J Struct Biol., 2019, 206, 170-182); 40GX and 40GY (Kenniston, J. A., et al., J Biol Chem., 2014, 289, 23596-23608); and 5F8T, 5F8X, and 5F8Z (Xu, M., et al.,).

Representative plasma kallikrein Targeting Ligands are provided in FIG. 1. Additional plasma kallikrein Targeting Ligands can be found in, for example, J Med Chem 61: 2823-2836 (2018), J Med Chem 55: 1171-80 (2012), U.S. Pat. Nos. 8,598,206, 9,738,655, Bioorg Med Chem Lett 16: 2034-6 (2006), U.S. Pat. No. 9,409,908, 10/144,746, U.S. Pat. No. 9,290,485, each of which is incorporated herein by reference.

### Lipoprotein Lipase

In some embodiments, the Target Extracellular Protein is human lipoprotein lipase (UniProtKB-P06858 (LIPL_HUMAN)). Lipoprotein lipase is a key enzyme in triglyceride metabolism. It catalyzes the hydrolysis of triglycerides from circulating chylomicrons and very low density lipoproteins (VLDL), and thereby plays an important role in lipid clearance from the blood stream, lipid utilization and storage. Lipoprotein lipase mediates margination of triglyceride-rich lipoprotein particles in capillaries. Lipoprotein lipase has been implicated in the development of cardiovascular disease and obesity.

The Protein Data Bank website provides the crystal structure of lipoprotein lipase bound to various compounds searchable by 6E7K (Birrane, G., et al., Proc Natl Acad Sci USA, 2018 116 1723-1732).

Representative lipoprotein lipase Targeting Ligands are provided in FIG. 1. Additional lipoprotein lipase Targeting Ligands can be found in, for example, J Med Chem 47: 400-10 (2004), which is incorporated herein by reference.

### Matrix Metallopeptidase 1 (MMP-1)

In some embodiments, the Target Extracellular Protein is human matrix metallopeptidase 1 (MMP-1) (UniProtKB-P03956 (MMP1_HUMAN)). MMP-1 cleaves collagens of types I, II, and III at one site in the helical domain. It also cleaves collagens of types VII and X. MMP-1 has been implicated in cardiovascular disease.

The Protein Data Bank website provides the crystal structure of MMP-1 searchable by 3SHI (Bertini, I., et al., FEBS Lett., 2012, 586, 557-567); as well as the crystal structure of MMP-1 bound to various compounds searchable by 4AUO (Manka, S. W., et al., Proc Natl Acad Sci U S A, 2012, 109, 12461); 3MA2 (Grossman, M., et al., Biochemistry, 2010, 49, 6184-6192); and 2JOT (Iyer, S., et al., J. Biol. Chem., 2007, 282, 364). Additionally, Iyer et al., provides insight into the crystal structure of an active form of MMP-1 (Iyer, S., et al., J Mol Biol., 2006, 362(1), 78-88); and Lovejoy et al., provides insight into the crystal structure of MMP1 and the selectivity of collagenase inhibitors (Lovejoy, B., et al., Nat Struct Mol Biol., 1999, 6, 217-221).

Representative MMP-1 Targeting Ligands are provided in FIG. 1. Additional MMP-1 Targeting Ligands can be found in, for example, Bioorg Med Chem Lett 5: 1415-1420 (1995), Bioorg Med Chem Lett 16: 2632-6 (2006), Bioorg Med Chem Lett 8: 837-42 (1999), Eur J Med Chem 60: 89-100 (2013), J Med Chem 54: 4350-64 (2011), Bioorg Med Chem Lett 8: 3251-6 (1999), J Med Chem 42: 4547-62 (1999), J Med Chem 61: 2166-2210 (2018), J Med Chem 41: 1209-17 (1998), which is incorporated herein by reference.

### Macrophage Migration Inhibitory Factor (MIF)

In some embodiments, the Target Extracellular Protein is human macrophage migration inhibitory factor (MIF) (UniProtKB-P14174 (MIF_HUMAN)). MIF is a pro-inflammatory cytokine involved in the innate immune response to bacterial pathogens. The expression of MIF at sites of inflammation suggests a role as mediator in regulating the function of macrophages in host defense. It counteracts the anti-inflammatory activity of glucocorticoids.

MIF has been implicated in tumor progression; systemic inflammation; atherosclerosis; rheumatoid arthritis; and systemic lupus erythematosus, among others.

The Protein Data Bank website provides the crystal structure of MIF searchable by 1MIF (Sun, H-W. et al., Proc Natl Acad Sci USA, 1996, 93, 5191-5196); as well as the crystal structure of MIF bound to various compounds searchable by 6PEG (Cirillo, P. F. et al.,); 5XEJ (Fukushima, K); 6FVE and 6FVH (Sokolov, A. V., et al., Biochemistry (Mosc), 2018, 83, 701-707); 6CB5, 6CBF, 6CBG, and 6CBH (Trivedi-Parmar, V., et al., Chem Med Chem., 2018, 13, 1092-1097); 6B1C, 6B1K, 6B2C, (Dawson, T. K., et al., ACS Med Chem Lett., 2017, 8, 1287-1291); 4Z15, 4Z1T and 4Z1U (Singh, A. K., et al, J Cell Mol Med., 2017, 21, 142-153); 5HVS and 5HVT 5 (Cisneros, J. A., et al., J Am Chem Soc., 2016, 138, 8630-8638); 4PKK (Pantouris, G., et al.,); 5J7P and 5J7Q (Cisneros, J. A., et al., Bioorg Med Chem Lett., 2016, 26, 2764-2767); 5B40 (Kimura, H., et al., Chem Biol., 2010, 17, 1282-1294); 4PLU, 4TRF, 4POH, and 4P01 (Pantouris, G., et al., Chem Biol., 2015, 22, 1197-1205); 4WR8 and 4WRB (Dziedzic, P., et al., J Am Chem Soc., 2015, 137 2996-3003); 4K9G (Ioannou, K., et al., Int J Oncol., 2014, 45, 1457-1468); 4OSF, 3WNR, 3WNS and 3WNT (Spencer, E. S., et al., Eur J Med Chem., 2015, 93, 501-510); 40YQ (Spencer, E. S. et al.,); 3SMB and 3SMC (Crichlow, G. V. et al., Biochemistry, 2012, 51, 7506-7514); 3U18 (Bai, F., et al., J Biol Chem., 2012, 287, 30653-30663); 4F2K (Tyndall, J. D. A., et al., Acta Crystallogr Sect F Struct Biol Cryst Commun., 2012, 68, 999-1002); 3IJG and 3IJJ (Cho, Y., et al., Proc Natl Acad Sci USA, 2010, 107, 11313-11318); 3L5P, 3L5R, 3L5S, 3L5T, 3L5U, and 3L5V (McLean, L. R. et al., Bioorg Med Chem Lett., 2010, 20, 1821-1824); 3JSF, 3JSG and 3JTU (McLean, L. R., et al., Bioorg Med Chem Lett., 2009, 19, 6717); 3HOF (Crawley, L., et al.); 3CE4 and 3DJI (Crichlow G. V., et al., Biochemistry, 2009, 48, 132-139); 3B9S (Winner, M. et al., Cancer Res., 2008, 68, 7253-7257); 2OOH, 2OOW and 2OOZ (Crichlow, G. V. et al., J Biol Chem., 2007, 282, 23089-23095); 1GCZ and 1GDO (Orita, M. et al., J Med Chem., 2001, 44, 540-547); and 1CA7, 1CGQ and 1P1G (Lubetsky, J. B. et al., Biochemistry, 1999, 38, 7346-7354). Additionally, Sun et al., provides insight into the crystal structure of MIF (Proc Natl Acad Sci U S A., 1996, 28; 93(11), 5191-6).

Representative MIF Targeting Ligands are provided in FIG. 1. Additional MIF Targeting Ligands can be found in, for example, ACS Med Chem Lett 8: 124-127 (2017), J Med Chem 44: 540-7 (2001), J Med Chem 52: 416-24 (2009), J Med Chem 50: 1993-7 (2007), which is incorporated herein by reference.

### Transforming Growth Factor-β2 (TGF-β2)

In some embodiments, the Target Extracellular Protein is human transforming growth factor-02 (TGF-β2) (UniProtKB-P61812 (TGFB2_HUMAN)). TGF-β2 is a multifunctional protein that regulates various processes such as angiogenesis and heart development. Once activated following release of LAP, TGF-beta-2 acts by binding to TGF-beta receptors (TGFBR1 and TGFBR2), which transduce signal. TGF- J2 expression in the tumor microenvironment has been associated with a poor prognosis, and is implicated in TGF-β2 mediated tumor suppression via T-cell exclusion. TGF- J2 expression has also been implicated in hematological malignancies and fibrosis.

The Protein Data Bank website provides the crystal structure of TGF-β2 searchable by 6I9J (Del Amo-Maestro L. et al., Sci Rep. 2019, 9, 8660-8660); as well as the crystal structure of TGF-32 bound to various compounds searchable by 1M9Z (Boesen, C. C., et al. Structure, 2002, 10, 913-919); 5QIN (Zhang, Y. et al., ACS Med Chem Lett., 2018, 9, 1117-1122); 5E8V, 5E8Y, 5E91 and 5E92 (Tebben, A. J. et al., Acta Crystallogr D Struct Biol., 2016, 72, 658-674); 4P7U (Wangkanont, K. et al., Protein Expr Purif, 2015, 115, 19-25); 4XJJ (Wangkanont et al.); and 1KTZ (Hart, P. J., et al., Nat Struct Biol., 2002, 9, 203-208).

Representative TGF-β2 Targeting Ligands are provided in FIG. 1.

### Thrombospondin-1 (TSP-1)

In some embodiments, the Target Extracellular Protein is human thrombospondin-1 (TSP-1) (UniProtKB-P61812 (TGFB2_HUMAN)). TSP1 acts as an angiogenesis inhibitor by stimulating endothelial cell apoptosis, inhibiting endothelial cell migration and proliferation, and regulating vascular endothelial growth factor bioavailability and activity. TSP1 affects tumor immune response, tumor cell behaviors including adhesion, invasion, migration, apoptosis, and proliferation.

TSP-1 expression has been implicated in a number of diseases, including in promoting certain cancers such as breast cancer, prostate cancer, melanoma, SCLC, osteosarcoma, cutaneous squamous cell carcinoma, oral squamous cell carcinoma, papillary thyroid carcinoma, thyroid cancer, medulloblastoma, and fibrotic disorders such as diabetes, liver fibrosis, and in multiple myeloma.

The Protein Data Bank website provides the crystal structure of TSP-1 searchable by 1LSL (Tan, K. et al., J Cell Biol., 2002, 159, 373-382); 2ES3 (Tan, K., et al., J Biol Chem., 2008, 283, 3932-3941); 1Z78 and 2ERF (Tan, K., et al., Structure, 2006, 14, 33-42); and 3R6B (Klenotic, P. A., et al., Protein Expr Purif., 2011, 80, 253-259); as well as the crystal structure of TSP-1 bound to various compounds searchable by 20UH and 2OUJ (Tan, K., et al., J Biol Chem., 2008, 283, 3932-3941); and 1ZA4 (Tan, K., et al., Structure, 2006, 14, 33-42).

Representative TSP-1 Targeting Ligands are provided in FIG. 1.

CD40 Ligand (CD40L)

In some embodiments, the Target Extracellular Protein is human CD40 ligand (CD40L) (UniProtKB-P29965 (CD40L_HUMAN)). CD40L is a cytokine that acts as a ligand to CD40/TNFRSF5. It costimulates T-cell proliferation and cytokine production. Its cross-linking on T-cells generates a costimulatory signal which enhances the production of IL4 and IL10 in conjunction with the TCR/CD3 ligation and CD28 costimulation. CD40L induces the activation of NF-kappa-B, as well as kinases MAPK8 and PAK2 in T-cells. It also induces tyrosine phosphorylation of isoform 3 of CD28. CD40L mediates B-cell proliferation in the absence of co-stimulus as well as IgE production in the presence of IL4, and is involved in immunoglobulin class switching.

The Protein Data Bank website provides the crystal structure of CD40L searchable by 1ALY (Karpusas, M., et al., Structure, 1995, 3, 1031-1039); as well as the crystal structure of CD40L bound to various compounds searchable by 3QD6 (An, H. J., et al., J Biol Chem., 2011, 286, 11226-11235); and 6BRB (Karnell, J. L., et al., Sci Transl Med., 2019, 11(489), 6584).

The expression of CD40L has been implicated in HIV-associated neurocognitive disorders and cardiovascular complications. Representative CD40L Targeting Ligands are provided in FIG. 1.

### Urokinase-Type Plasminogen Activator (UPA)

In some embodiments, the Target Extracellular Protein is human urokinase-type plasminogen activator (UPA) (UniProtKB-P00749 (UROK_HUMAN)). Urokinase-type plasminogen activator (uPA), is a serine protease present in the blood and in the extracellular matrix of many tissues. The primary physiological substrate of this enzyme is plasminogen, which is an inactive form (zymogen) of the serine protease plasmin. Activation of plasmin triggers a proteolytic cascade that, depending on the physiological environment, participates in thrombolysis or extracellular matrix degradation. This cascade had been involved in vascular diseases and cancer progression. Elevated expression levels of urokinase and several other components of the plasminogen activation system are found to be correlated with tumor malignancy.

The Protein Data Bank website provides the crystal structure of UPA bound to various compounds searchable by 5ZA7, 5ZAJ, 5ZA8, 5ZA9, 5ZAE, 5ZAF, 5ZAG, 5ZAH, and 5ZC5 (Buckley, B. J. et al., J Med Chem., 2018, 61, 8299-8320); 5LHP, 5LHQ, 5LHR, and 5LHS (Kromann-Hansen, T. et al., Sci Rep., 2017, 7, 3385-3385); 2VNT (Fish, P. V. et al. J Med Chem., 2007, 50, 2341); 1OWD, 1OWE, 1OWH, 1OWI, 1OWJ, and 1OWK (Wendt, M. D. et al., J Med Chem., 2004, 47, 303-324); 1SQA, 1SQO, and 1SQT (Wendt, M. D., et al., Bioorg Med Chem Lett., 2004, 14, 3063-3068); 1U6Q (Bruncko, M. et al., Bioorg Med Chem Lett., 2005, 15, 93-98); 3OX7, 3OY5 and 3OY6 (Jiang, L. G. et al., J Mol Biol., 2011, 412, 235-250); 4OS1, 40S2, 4OS4, 4OS5, 4OS6 and 4OS7 (Chen, S. et al., Nat Chem., 2014, 6, 1009-1016); 3IG6 (West, C. W. et al., Bioorg Med Chem Lett., 2009, 19, 5712-5715); 4X0W and 4X1P (Jiang, L. et al., Int J Biochem Cell Biol., 2015, 62, 88-92); 4X1N, 4X1Q, 4X1R and 4X1S (Zhao, B. et al., PLoS One, 2014, 9, e115872-e115872); 5WXO and 5WXP (Jiang, L. et al., Biochim Biophys Acta., 2018, 1862, 2017-2023); 4MNV, 4MNW, 4MNX, and 4MNY (Chen, S., et al., Angew Chem Int Ed Engl., 2014, 53, 1602-1606); 4GLY (Chen, S., et al., J Am Chem Soc., 2013, 135, 6562-6569); 4JK5 and 4JK5 (Chen, S., et al., Chembiochem., 2013, 14, 1316-1322); 3QN7 (Angelini, A. et al., ACS Chem Biol., 2012, 7, 817-821); 2NWN (Zhao, G. et al., J Struct Biol., 2007, 160, 1-10); 6NMB (Wu, G. et al., Blood Adv., 2019, 3, 729-733); 1W0Z, 1W10, 1W11, 1W12, 1W13, and 1W14 (Zeslawska, E. et al., J Mol Biol., 2003, 328, 109); 4DVA (Jiang, L et al., Biochem J., 2013, 449, 161-166); 6A8G 6A8N (Wang, D. et al., J Med Chem., 2019, 62, 2172-2183); 2VIN, 2VIO, 2VIP, 2VIQ, 2VIV, and 2VIW (Frederickson, M. et al., J Med Chem., 2008, 51, 183); 1EJN (Speri, S., et al., Proc Natl Acad Sci USA, 2000, 97, 5113-5118); 3PB1 (Lin, Z. et al., J Biol Chem., 2011, 286, 7027-7032); 3U73 (Xu, X. et al., J Mol Biol., 2012, 416, 629-641); 1C5W, 1C5X, 1C5Y and IC5Z (Katz, B. A., et al., Chem Biol., 2000, 7, 299-312); 5XG4 (Xue, G. et al., Food Funct., 2017, 8, 2437-2443); 5WXF (Jiang, L. et al., Biochim Biophys Acta., 2018, 1862, 2017-2023); 5WXS, 4ZKS, 5WXQ, 5WXT, 5YC6, 5YC7, 5Z1C, (Jiang, L. et al.); 4H42 (Yu, H. Y. et al.,); 6AG3 and 6AG9 (Buckley, B. et al); 3KGP, 3KHV, 3KID, 3M61, 3MHW, and 3MWI (Jiang, L. G. et al.,); 4ZKN, 4ZKO and 4ZKR (Jiang, L. et al.); 2O8T, 2O8U, 2O8W (Zhao, G. et al.,); and 4FU7, 4FU8, 4FU9, 4FUB, 4FUC, 4FUD, 4FUE, 4FUF, 4FUG, 4FUH, 4FUI, and 4FUJ (Kang, Y. N. et al.).

Representative UPA Targeting Ligands are provided in FIG. 1. Additional UPA Targeting Ligands are provided in, for example, J Med Chem 38: 1511-22 (1995), Bioorg Med Chem Lett 11: 2253-6 (2001), Bioorg Med Chem Lett 14: 3063-8 (2004), J Med Chem 52: 3159-65 (2009), CSAR 1: (2012), Bioorg Med Chem 22: 3187-203 (2014), J Med Chem 50: 2341-51 (2007), J Mol Biol 329: 93-120 (2003), Bioorg Med Chem Lett 2:1399-1404 (1992), J Med Chem 35: 4297-305 (1992), J Med Chem 35: 4150-9 (1992), J Med Chem 49: 5785-93 (2006), Bioorg Med Chem 23: 3696-704 (2015), Bioorg Med Chem Lett 10: 983-7 (2000), J Med Chem 49: 5785-93 (2006), each of which is incorporated by reference herein.

### Plasminogen Activator, Tissue Type (TPA)

In some embodiments, the Target Extracellular Protein is human plasminogen activator, tissue type (TPA) (UniProtKB-P00750 (TPA_HUMAN)). TPA converts the abundant, but inactive, zymogen plasminogen to plasmin by hydrolyzing a single Arg-Val bond in plasminogen. By controlling plasmin-mediated proteolysis, it plays an important role in tissue remodeling and degradation, in cell migration and many other physiopathological events. TPA plays a direct role in facilitating neuronal migration. PLA has been shown activated in various cancers including oral malignancy.

The Protein Data Bank website provides the crystal structure of TPA searchable by 1VR1 (Dekker, R. J. et al., J Mol Biol., 1999, 293, 613-627); as well as the crystal structure of TPA bound to various compounds searchable by 1RTF (Lamba, D. et al., J Mol Biol., 1996, 258, 117-135); 1ASH (Renatus, M. et al., J Biol Chem., 1997, 272, 21713-21719); and 1BDA (Renatus, M. et al., EMBO J., 1997, 16, 4797-4805).

Representative TPA Targeting Ligands are provided in FIG. 1. Additional TPA Targeting Ligands are provided in, for example, Bioorg Med Chem Lett 15: 4411-6 (2005), Bioorg Med Chem Lett 13: 2781-4 (2003), Bioorg Med Chem Lett 6: 2913-2918 (1996), J Med Chem 44: 2753-71 (2001), J Med Chem 41: 5445-56 (1999), Bioorg Med Chem Lett 12: 3183-6 (2002), U.S. patent Ser. No. 10/118,930, J Biol Chem 285: 7892-902 (2010), each of which is incorporated by reference herein.

### Plasminogen (PLG)

In some embodiments, the Target Extracellular Protein is human plasminogen (PLG) (UniProtKB-P00747 (PLMN_HUMAN)). PLG dissolves the fibrin of blood clots and acts as a proteolytic factor in a variety of other processes including embryonic development, tissue remodeling, tumor invasion, and inflammation. It activates the urokinase-type plasminogen activator, collagenases and several complement zymogens, such as C1 and C5. Its role in tissue remodeling and tumor invasion may be modulated by CSPG4.

The Protein Data Bank website provides the crystal structure of PLG searchable by 1DDJ (Wang, X. et al., J. Mol. Biol., 2000, 295, 903-914); and 4DUR and 4DUU (Law, R. H. P., et al., Cell Rep., 2012, 1, 185-190).

Representative PLG Targeting Ligands are provided in FIG. 1. Additional PLG Targeting Ligands are provided in, for example, J Med Chem 35: 4297-305 (1992), J Med Chem 38: 1511-22 (1995), J Med Chem 56: 820-31 (2013), U.S. Pat. Nos. 8,598,206, 8,921,319, J Med Chem 55: 1171-80 (2012), Bioorg Med Chem Lett 12: 3183-6 (2002), Bioorg Med Chem 23: 3696-704 (2015), Bioorg Med Chem Lett 13: 723-8 (2003), Bioorg Med Chem Lett 7: 331-336 (1997), each of which is incorporated by reference herein.

### Plasminogen Activator Inhibitor-1 (PAI-1)

In some embodiments, the Target Extracellular Protein is human plasminogen activator inhibitor 1 (PAI-1) (UniProtKB-P05121 (PAI1_HUMAN)). PAI-1 is a serine protease inhibitor, and a primary inhibitor of tissue-type plasminogen activator (PLAT) and urokinase-type plasminogen activator (PLAU). As PLAU inhibitor, it is involved in the regulation of cell adhesion and spreading, and acts as a regulator of cell migration, independently of its role as protease inhibitor. Overexpression of PAI-1 favors angiogenesis, metastasis, and poor prognosis in tumors, including, but not limited to, oral cancers and breast cancers.

The Protein Data Bank website provides the crystal structure of PAI-1 searchable by 3Q02 and 3Q03 (Jensen, J. K. et al., J Biol Chem., 2011, 286, 29709-29717); 1B3K (Sharp, A. M. et al., Structure, 1999, 7, 111-118); 1C5G (Tucker, H. M. et al., Nat Struct Biol., 1995, 2, 442-445); 1DVM (Stout, T. J. et al., Biochemistry, 2000, 39, 8460-8469); and 3UT3 (Lin, Z. H. et al.,); as well as the crystal structure of PAI-1 bound to various compounds searchable by 4AQH (Fjellstrom, O. et al., J Biol Chem., 2013, 288, 873); 3R4L (Jankun, J. et al., Int J Mol Med., 2012, 29 61-64); 1A7C (Xue, Y., et al., Structure, 1998, 6, 627-636); 1OC0 (Zhou, A. et al., Nat Struct Biol., 2003, 10, 541); 6I8S (Vousden, K. A. et al., Sci Rep., 2019, 9, 1605-1605); 4G80 and 4G8R (Li, S. H. et al., Proc Natl Acad Sci USA, 2013, 110, E4941-E4949); 6GWQ, 6GWN and 6GWP (Sillen, M. et al., J Thromb Haemost, 2019); and 4IC0 (Hong, Z. B. et al.,).

Representative PAI-1 Targeting Ligands are provided in FIG. 1. Additional PAI-1 Targeting Ligands are provided in, for example, J Biol Chem 285: 7892-902 (2010), U.S. Pat. No. 9,120,744, Bioorg Med Chem Lett 13: 3361-5 (2003), Bioorg Med Chem Lett 12: 1063-6 (2002), Bioorg Med Chem Lett 13: 1705-8 (2003), Bioorg Med Chem Lett 11: 2589-92 (2001), U.S. Pat. No. 9,718,760, each of which is incorporated by reference herein.

### Placenta Growth Factor (PIGF)

In some embodiments, the Target Extracellular Protein is human placental growth factor (PGF) (UniProtKB-P49763 (PLGF_HUMAN)). PGF is growth factor active in angiogenesis and endothelial cell growth, stimulating their proliferation and migration. It binds to the receptor FLT1/VEGFR-1. Isoform PlGF-2 binds NRP1/neuropilin-1 and NRP2/neuropilin-2 in a heparin-dependent manner. PGF also promotes cell tumor growth, and has been implicated in age-related macular degeneration (AMD) and choroidal neovascularization (CNV).

The Protein Data Bank website provides the crystal structure of PIGF searchable by 1FZV (Iyer, S. et al., J Biol Chem., 2001, 276, 12153-12161); as well as the crystal structure of PIGF bound to various compounds searchable by 1RV6 (Christinger, H. W., J Biol Chem., 2004, 279, 10382-10388). Additionally, De Falco provides insight into the discovery and biological activity of placenta growth factor (De Falco, Exp Mol Med., 2012, 44, 1-9).

Representative PGF Targeting Ligands are provided in FIG. 1. Additional PGF Targeting Ligands are provided in, for example, J Med Chem 54: 1256-65 (2011), J Nat Prod 76: 29-35 (2013), each of which is incorporated by reference herein.

### Phospholipase A2, Group IB (PA21B)

In some embodiments, the Target Extracellular Protein is human phospholipase A2, Group IB (PA21B) (UniProtKB-P04054 (PA21B_HUMAN)). PA21B cleaves phospholipids preferentially at the sn-2 position, liberating free fatty acids and lysophospholipids. PA21B has been implicated in a number of diseases, including cardiovascular diseases, atherosclerosis, immune disorders and cancer.

The Protein Data Bank website provides the crystal structure of PA21B searchable by 3FVJ and 3FVI (Pan, Y. H. et al., Biochim. Biophys. Acta., 2010, 1804, 1443-1448).

Representative PA21B Targeting Ligands are provided in FIG. 1. Additional PA21B Targeting Ligands are provided in, for example, J Med Chem 39: 3636-58 (1996), Chembiochem 4: 181-5 (2003), J Med Chem 39: 5159-75 (1997), J Med Chem 51: 4708-14 (2008), each of which is incorporated by reference herein.

### Phospholipase A2, Group IIA (PA2GA)

In some embodiments, the Target Extracellular Protein is human phospholipase A2, Group IIA (PA2GA) (UniProtKB-P04054 (PA21B_HUMAN)). PA2GA catalyzes the calcium-dependent hydrolysis of the 2-acyl groups in 3-sn-phosphoglycerides. It is thought to participate in the regulation of phospholipid metabolism in biomembranes including eicosanoid biosynthesis. Independent of its catalytic activity, it also acts as a ligand for integrins. PA2GA Induces cell proliferation in an integrin-dependent manner. PA2GA has been implicated in a number of diseases, including cardiovascular diseases, atherosclerosis, immune disorders, and cancer. The Protein Data Bank website provides the crystal structure of PA2GA bound to various compounds searchable by 2ARM and 1SV3 (Singh, N. et al., Proteins, 2006, 64, 89-100); 5G3M and 5G3N (Giordanetto, F., et al. ACS Med Chem Lett., 2016, 7, 884); 1KQU (Jansford, K. A., et al., Chembiochem., 2003, 4, 181-185); and 1ZYX (Singh, N. et al.,). Additionally, Singh et al., provides insight into the crystal structure of the complexes of a group IIA phospholipase A2 with two natural anti-inflammatory agents, anisic acid, and atropine reveal a similar mode of binding (Singh, N. et al., Proteins, 2006, 64(1):89-100); and Kitadokoro et al also provides insight into the crystal structure of human secretory phospholipase A2-IIA complex with the potent indolizine inhibitor 120-1032 (Kitadokoro, K. et al., J Biochem., 1998, 123(4), 619-23).

Representative PA2GA Targeting Ligands are provided in FIG. 1. Additional PA2GA Targeting Ligands are provided in, for example, J Med Chem 48: 893-6 (2005), J Med Chem 39: 5159-75 (1997), each of which is incorporated by reference herein.

### Factor B

In some embodiments, the Target Extracellular Protein is human Complement factor B (UniProtKB-P00751 (CFAB_HUMAN)). Complement factor B, which is part of the alternate pathway of the complement system, is cleaved by factor D into 2 fragments: Ba and Bb. Bb, a serine protease, then combines with complement factor 3b to generate the C3 or C5 convertase. It has also been implicated in proliferation and differentiation of preactivated B-lymphocytes, rapid spreading of peripheral blood monocytes, stimulation of lymphocyte blastogenesis and lysis of erythrocytes. Ba inhibits the proliferation of preactivated B-lymphocytes.

The Protein Data Bank website provides the crystal structure of Complement Factor B searchable by 20K5 (Milder, F. J., et al., Nat Struct Mol Bio 2007, 14, 224-228); as well as the crystal structure of Complement factor B bound to various compounds searchable by 6QSW, 6QSX, and 6RAV (Schubart, A., et al., Proc Natl Acad Sci 2019, 116, 7926-7931); 6T8U, 6T8W, and 6T8V (Mainolfi, N., et al, J Med Chem 2020, 63, 5697-5722); and 7JTN (Xu, X., et al., J Immunol 2021, 206, doi:10.4049/jimmunol.2001260).

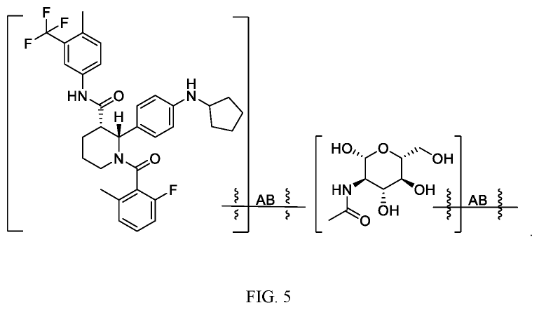

Representative Complement Factor B Targeting Ligands are provided in FIG. 5. Additional Complement Factor B Targeting Ligands are provided in, for example, U.S. Pat. No. 9,682,968B2, U.S. Pat. No. 9,475,806B2, U.S. Pat. No. 9,452,990B2, Proc Natl Acad Sci 116: 7926-7931 (2019), J Med Chem 52: 6042-6052 (2009), and J Med Chem 63: 5697-5722 (2020), each of which is incorporated by reference herein.

In certain embodiments the Extracellular Targeting Ligand is selected from:

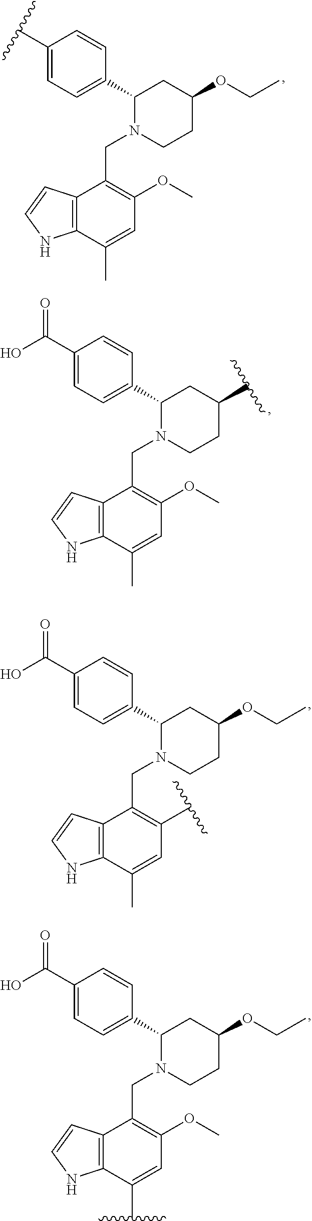 [SMILES: *c1ccc([C@@H]2C[C@@H](OCC)CCN2Cc2c(OC)cc(C)c3[nH]ccc23)cc1 ; *[C@H]1CCN(Cc2c(OC)cc(C)c3[nH]ccc23)[C@H](c2ccc(C(=O)O)cc2)C1 ; *c1cc(C)c2[nH]ccc2c1CN1CC[C@H](OCC)C[C@H]1c1ccc(C(=O)O)cc1 ; *c1cc(OC)c(CN2CC[C@H](OCC)C[C@H]2c2ccc(C(=O)O)cc2)c2cc[nH]c12]

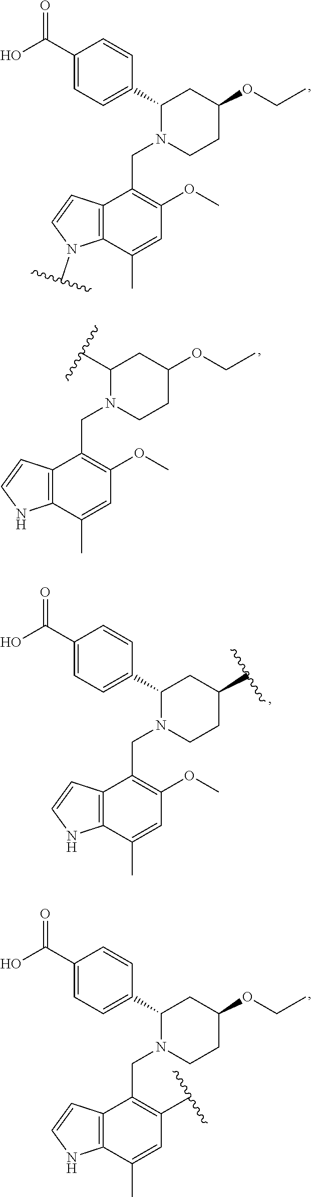 [SMILES: *n1ccc2c(CN3CC[C@H](OCC)C[C@H]3c3ccc(C(=O)O)cc3)c(OC)cc(C)c21 ; *C1CC(OCC)CCN1Cc1c(OC)cc(C)c2[nH]ccc12 ; *[C@H]1CCN(Cc2c(OC)cc(C)c3[nH]ccc23)[C@H](c2ccc(C(=O)O)cc2)C1 ; *c1cc(C)c2[nH]ccc2c1CN1CC[C@H](OCC)C[C@H]1c1ccc(C(=O)O)cc1]

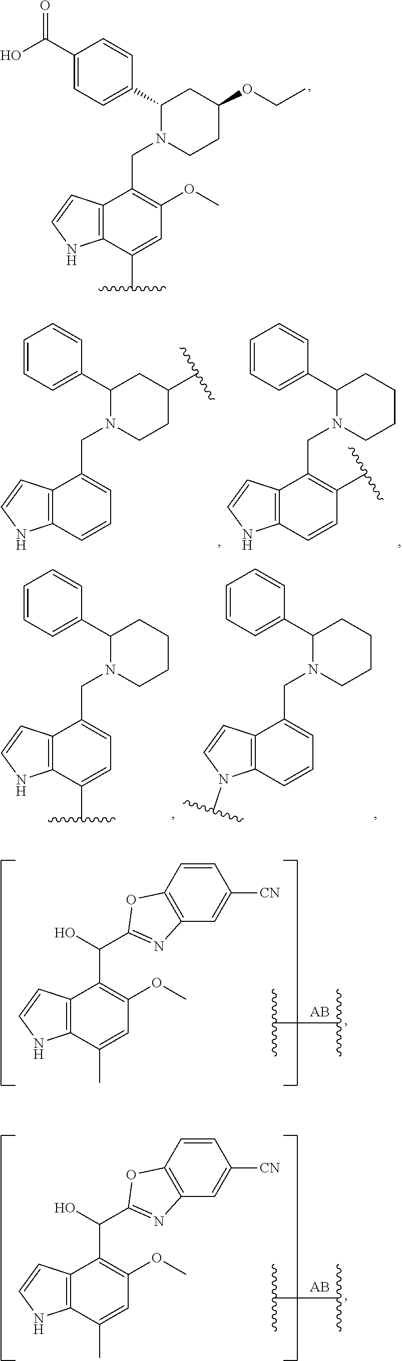

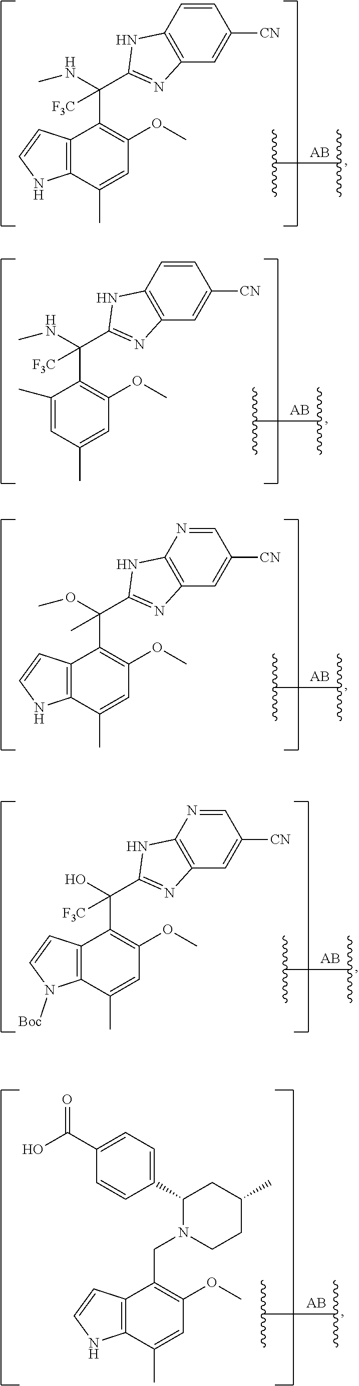

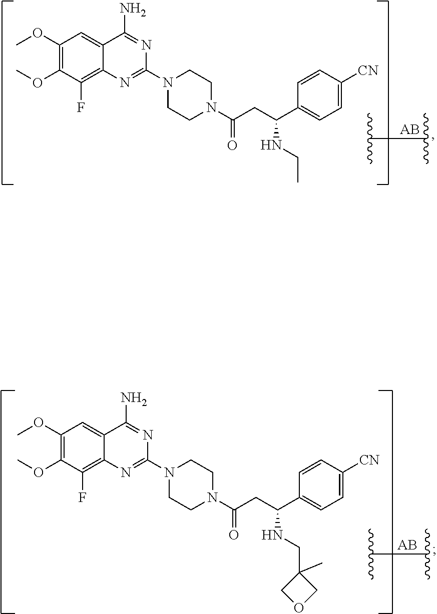

each of which is optionally substituted with 1, 2, 3, or 4 substituents independently selected from R²¹.

In certain embodiments the Factor B Targeting Ligand is selected from a ligand described in: Mainolfi, N. et. al. Discovery of 4-((2 S,4 S )-4-Ethoxy-1-((5-Methoxy-7-Methyl-1H-Indol-4-Yl)Methyl)Piperidin-2-Yl)Benzoic Acid (LNP023), a Factor B Inhibitor Specifically Designed To Be Applicable to Treating a Diverse Array of Complement Mediated Diseases. J. Med. Chem. 2020, 63 (11), 5697-5722; WO2020/016749; WO2018/005552; WO2013/192345; or WO2015009616.

In certain embodiments the factor B Targeting Ligand-linker is selected from:

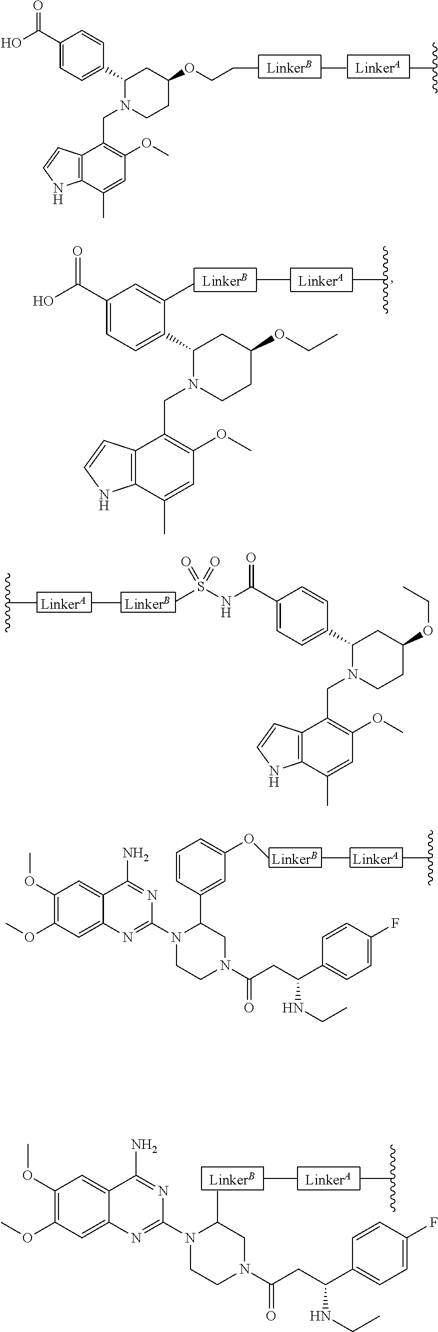

In certain embodiments the compound of the present invention is selected from the following compounds or a bi- or tri-dentate version thereof:

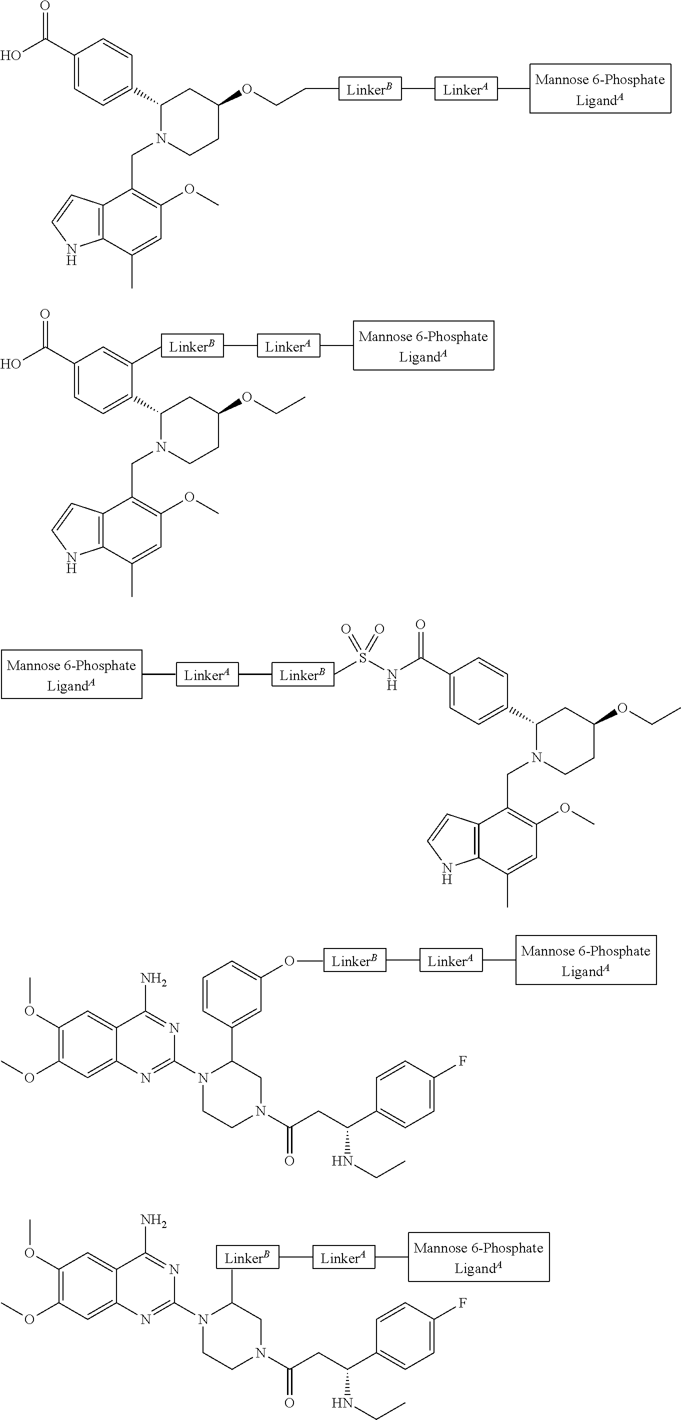

### Factor D

In some embodiments, the Target Extracellular Protein is human Complement factor D (UniProtKB-P00746 (CFAD_HUMAN)). Factor D cleaves factor B when the latter is complexed with factor C3b, activating the C3bbb complex, which then becomes the C3 convertase of the alternate pathway. Its function is homologous to that of C1s in the classical pathway.

The Protein Data Bank website provides the crystal structure of Complement factor D bound to various compounds searchable by 6FTZ, 6FUT, 6FUH, 6FUG, 6FUJ, and 6FUI (Vulpetti, A., et al., ACS Med Chem Lett 2018, 9, 490-495); 5TCA and 5TCC (Yang, C. Y., et al., ACS Med Chem Lett 2016, 7, 1092-1096); 5MT4 (Vulpetti, A., et al., J Med Chem 2017, 60, 1946-1958); 1DFP (Cole, L. B., et al., Acta Crystallogr D Biol Crystallogr 1997, 53, 143-150); 1DIC (Cole, L. B., et al., Acta Crystallogr D Biol Crystallogr 1998, 54, 711-717); 6QMR and 6QMT (Karki, R. G., et al., J Med Chem 2019, 62, 4656-4668).

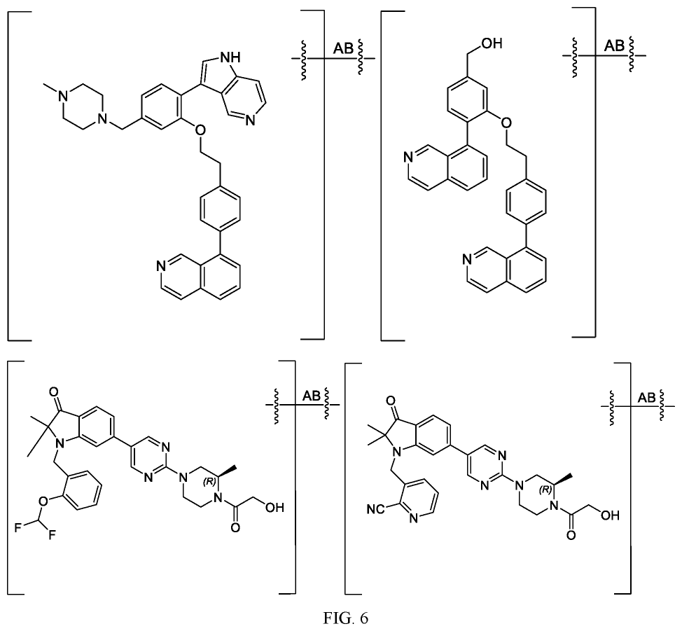

Representative Complement factor D Targeting Ligands are provided in FIG. 6. Additional Complement Factor D Targeting Ligands are provided in, for example, J Med Chem 60: 5717-5735 (2017), Nat Chem Biol 12: 1105-1110 (2016), U.S. Pat. Nos. 9,598,446B2, 9,643,986B2, 9,663,543B2 9,695,205B2, 9,732,103B2, 9,732,104B2, 9,758,537B2, 9,796,741B2, 9,828,396B2, U.S. patent Ser. Nos. 10/000,516B2, 10/005,802B2, 10/011,612B2, 10/081,645B2, 10/087,203B2, 10/092,584B2, 10/100,072B2, 10/106,563B2, 10/138,225B2, 10/189,869B2, 10/253,053B2, 10/287,301B2, 10/301,336B2, 10/370,394B2, 10/385,097B2, 10/428,094B2, 10/428,095B2, 10/464,956B2, 10/550,140B2, 10/660,876B2, 10/662,175B2, 10/689,409B2, 10/807,952B2, 10/822,352B2, U.S. Pat. No. 9,464,081B2, and Hematological 102: 466-475 (2017), each of which is incorporated by reference herein.

In certain embodiments the Extracellular Targeting Ligand is selected from:

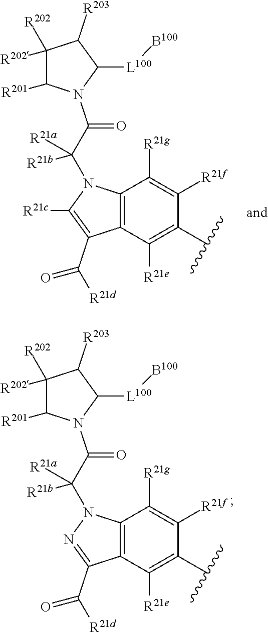

wherein:
R^{21a}, R^{21b}, R^{21c}, R^{21d}, R^{21e}, R^{21f}, and R^{21g} are independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, aryl, heteroaryl, heterocyclyl, —SR³, —C(O)OR³, —C(O)NR⁶NR⁷, —OR³, and heterocycle;
R²⁰¹, R²⁰², R²⁰²′, and R²⁰³ are independently selected from hydrogen, halogen, hydroxyl, nitro, cyano, amino, C₁-C₆alkyl, C₂-C₆alkenyl, C₁-C₆alkoxy, C₂-C₆alkynyl, C₂-C₆alkanoyl, C₁-C₆thioalkyl, hydroxyC₁-C₆alkyl, aminoC₁-C₆alkyl, —C₀-C₄alkylNRWR¹⁰, —C(O)OR⁹, —OC(O)R⁹, —NR⁹C(O)R¹⁰, —C(O)NR⁹R¹⁰, —OC(O)NR⁹R¹⁰, —O(heteroaryl), —NR⁹C(O)OR¹⁰, C₁-C₂haloalkyl, —C₀-C₄alkyl(C₃-C₇cycloalkyl) and —O—C₀-C₄alkyl(C₃-C₇cycloalkyl), and C₁-C₂haloalkoxy, where R²⁰⁹ and R²¹⁰ are independently chosen at each occurrence from hydrogen, C₁-C₆alkyl, and (C₃-C₇cycloalkyl)C₀-C₄alkyl;
or R²⁰² and R²⁰²′ may be taken together to form a 3- to 6-membered spiro ring optionally substituted with 1 or more substituents independently chosen from halogen, hydroxyl, cyano, —COOH, C₁-C₄alkyl (including in particular methyl), C₂-C₄alkenyl, C₂-C₄alkynyl, C₁-C₄alkoxy, C₂-C₄alkanoyl, hydroxyC₁-C₄alkyl, (mono- and di-C₁-C₄alkylamino)C₀-C₄alkyl, —C₀-C₄alkyl(C₃-C₇cycloalkyl), —O—C₀-C₄alkyl(C₃-C₇cycloalkyl), C₁-C₂haloalkyl, and C₁-C₂haloalkoxy.
or R²⁰¹ and R²⁰² may be taken together to form a 3-membered carbocyclic ring, optionally substituted with 1, 2, or 3 substituents selected from R²¹.
or R²⁰¹ and R²⁰² may be taken together to form a 4- to 6-membered carbocyclic ring or a 4- to 6-membered heterocyclic ring containing 1 or 2 heteroatoms independently chosen from N, O, and S, optionally substituted with 1, 2, or 3 substituents selected from R²¹.
R²⁰² and R²⁰³ may be taken together to form a 3- to 6-membered carbocyclic ring or a 3- to 6-membered heterocyclic ring optionally substituted with 1, 2, or 3 substituents selected from R²¹.
L¹⁰⁰ is selected from

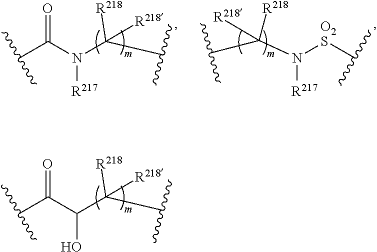

wherein R²¹⁷ is hydrogen or C₁-C₆alkyl and R²¹⁸ and R^{218′} are independently chosen from hydrogen, halogen, hydroxymethyl, and methyl; and m is 0, 1, 2, or 3;
B¹⁰⁰ is a cycloalkyl, heterocycle group having 1, 2, 3, or 4 heteroatoms independently selected from N, O, and S, a C₂-C₆alkenyl, C₂-C₆alkynyl group, —(C₀-C₄alkyl)(aryl), —(C₀-C₄alkyl)(heteroaryl), or —(C₀-C₄alkyl)(biphenyl), each of which is optionally substituted with 1, 2, 3, or 4 substituents independently selected from R²¹.

In certain embodiments the Extracellular Targeting Ligand is selected from:

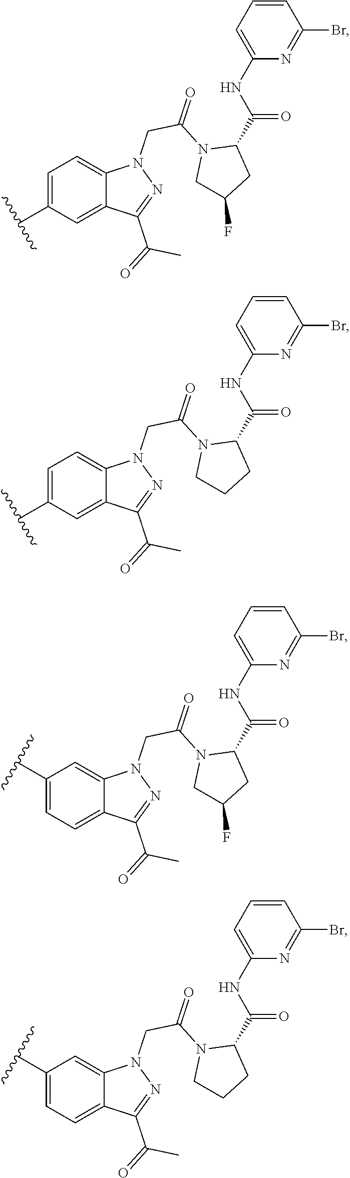 [SMILES: *c1ccc2c(c1)c(C(C)=O)nn2CC(=O)N1C[C@H](F)C[C@H]1C(=O)Nc1cccc(Br)n1 ; *c1ccc2c(c1)c(C(C)=O)nn2CC(=O)N1CCC[C@H]1C(=O)Nc1cccc(Br)n1 ; *c1ccc2c(C(C)=O)nn(CC(=O)N3C[C@H](F)C[C@H]3C(=O)Nc3cccc(Br)n3)c2c1 ; *c1ccc2c(C(C)=O)nn(CC(=O)N3CCC[C@H]3C(=O)Nc3cccc(Br)n3)c2c1]

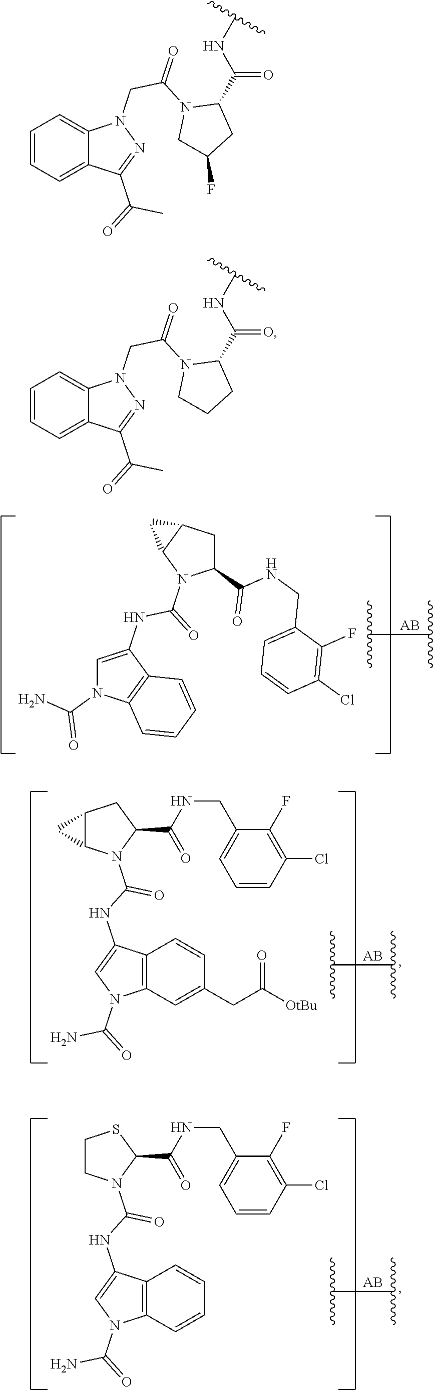

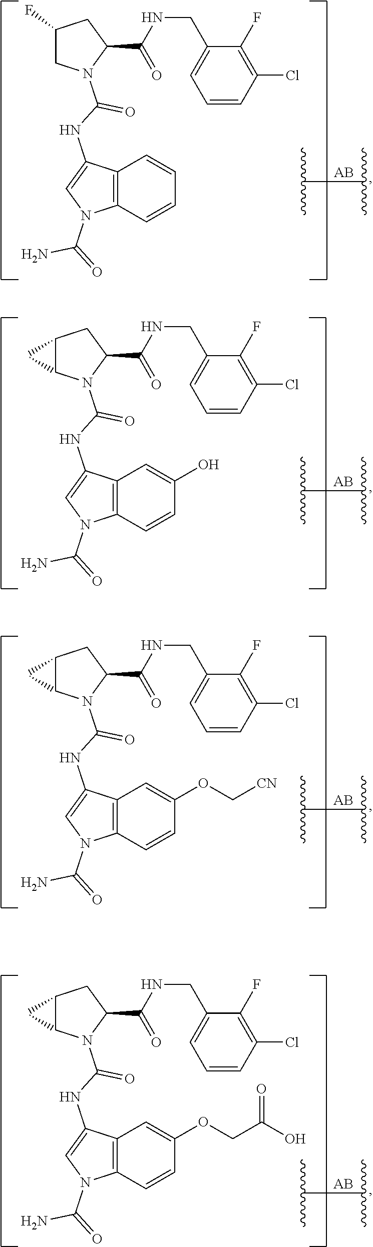

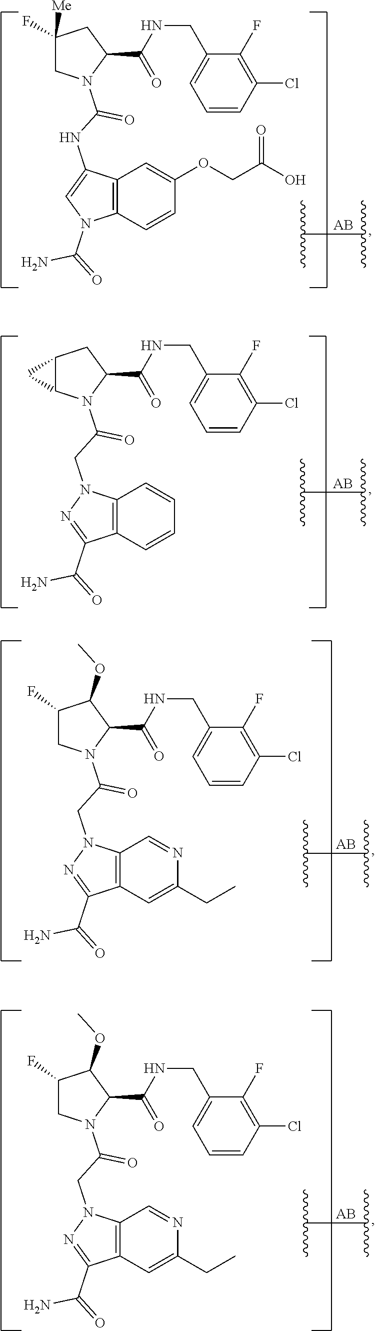

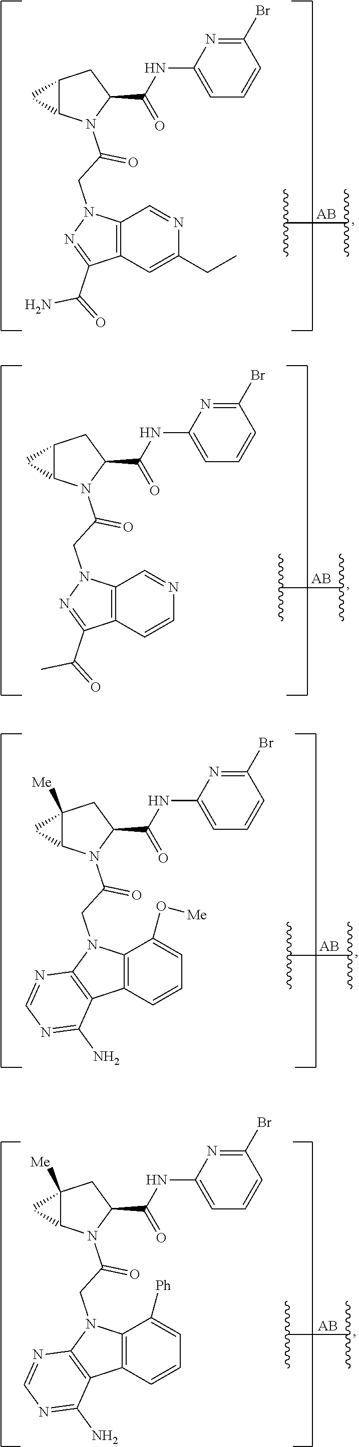

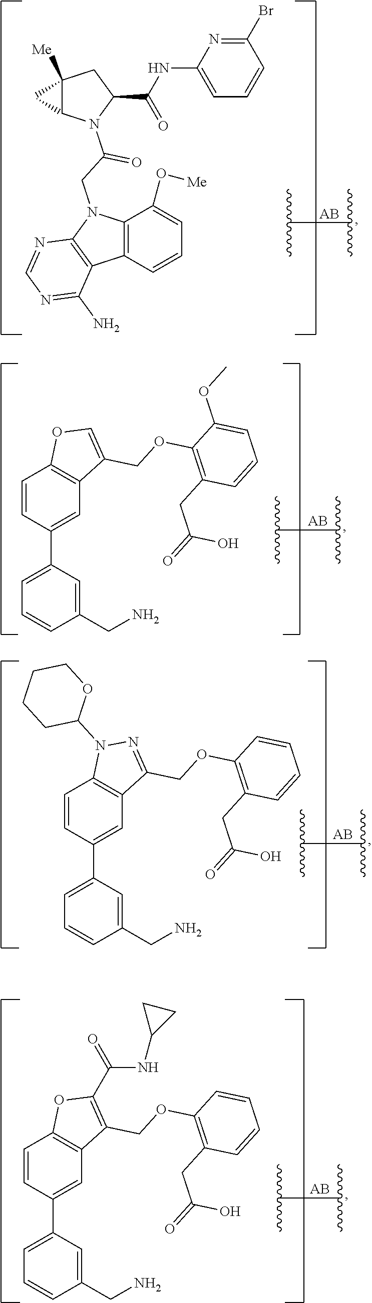

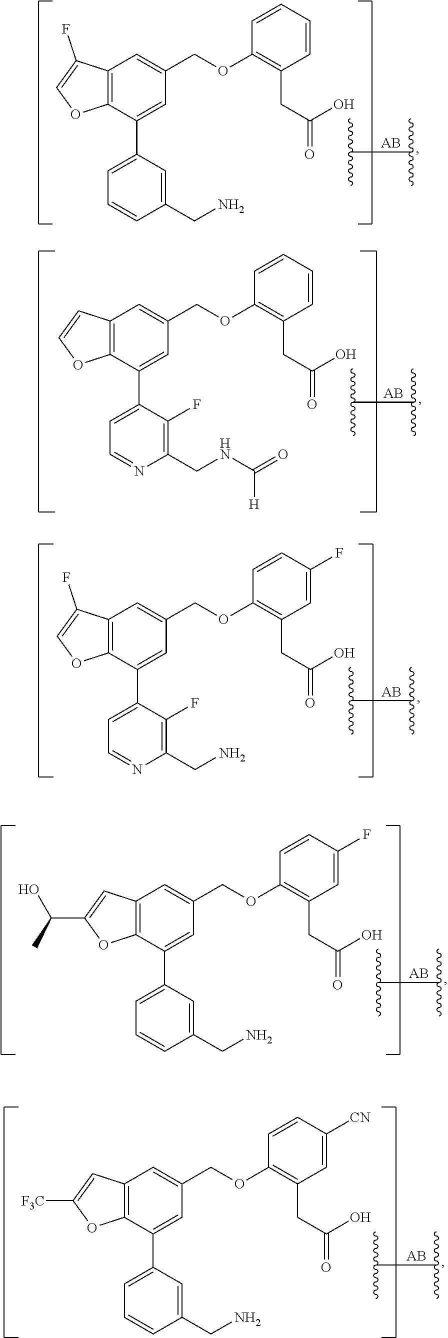

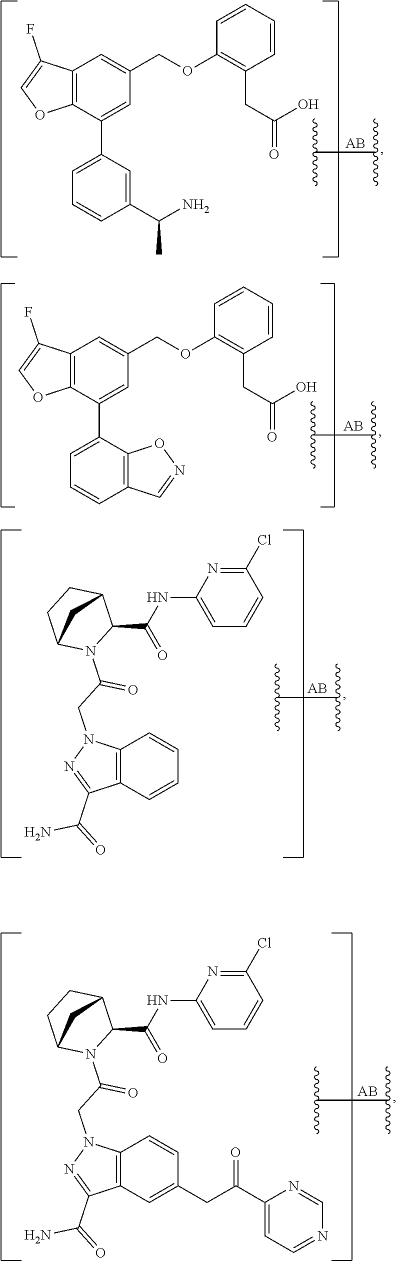

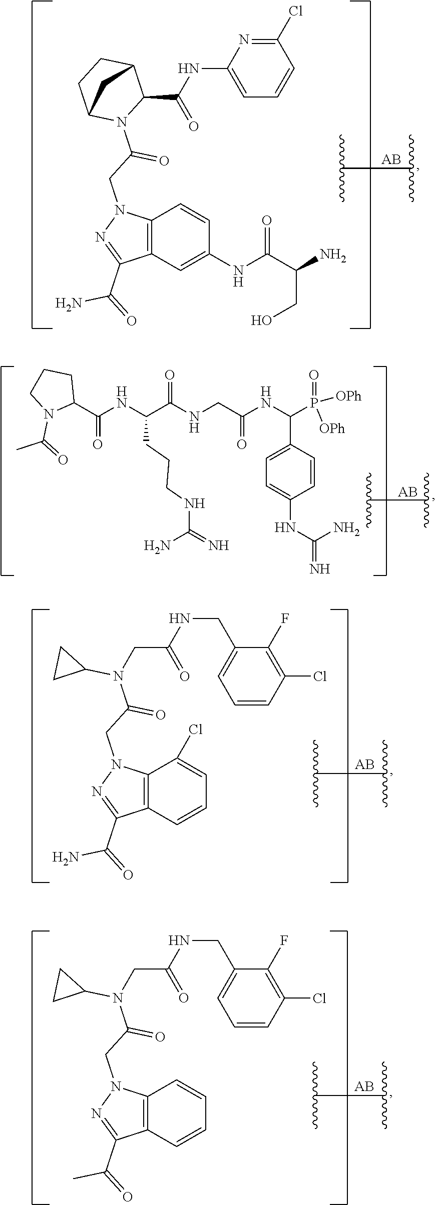

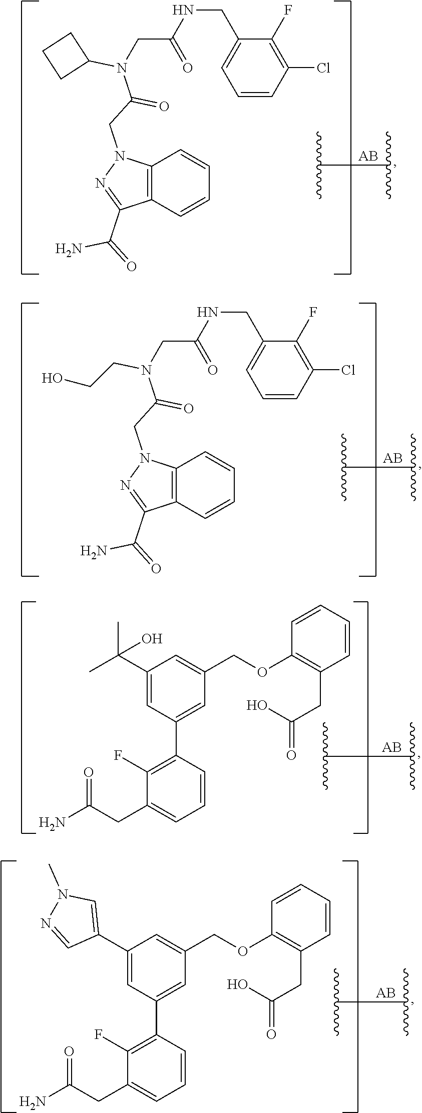

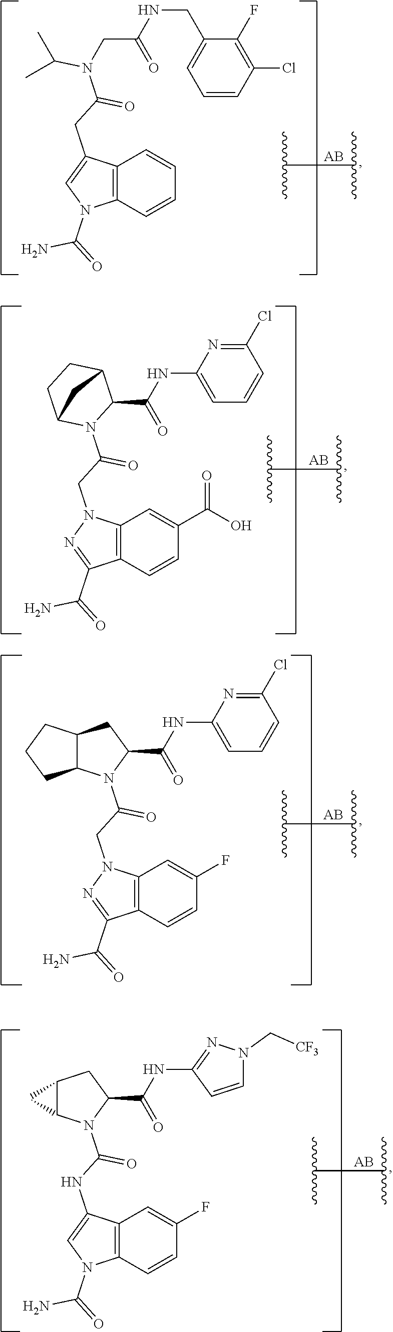

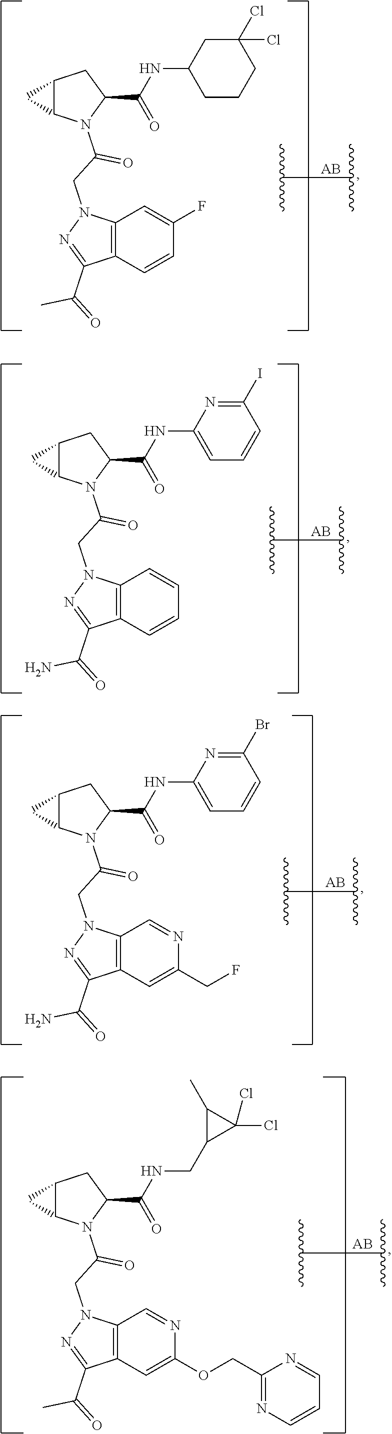

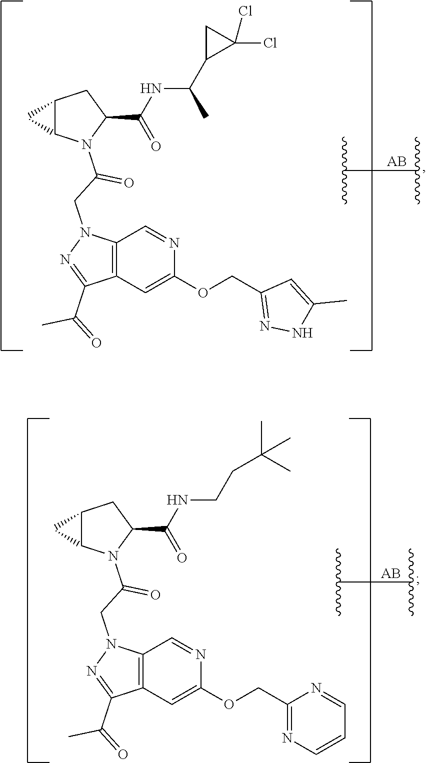

each of which is optionally substituted with 1, 2, 3, or 4 substituents independently selected from R²¹ and is attached to the linker at a location allowed by valence.

In certain embodiments the Factor D Targeting Ligand is selected from a ligand described in: U.S. Pat. Nos. 9,796,74; 10,011,612; WO2018/160889; WO2019/195720; WO2019/057946; Karki, R. G. et al. Design, Synthesis, and Preclinical Characterization of Selective Factor D Inhibitors Targeting the Alternative Complement Pathway. J. Med. Chem. 2019, 62 (9), 4656-4668; or Belanger, D. B. et al.; WO2015/009977.

In certain embodiments the complement factor D targeting ligand-linker- is selected from:

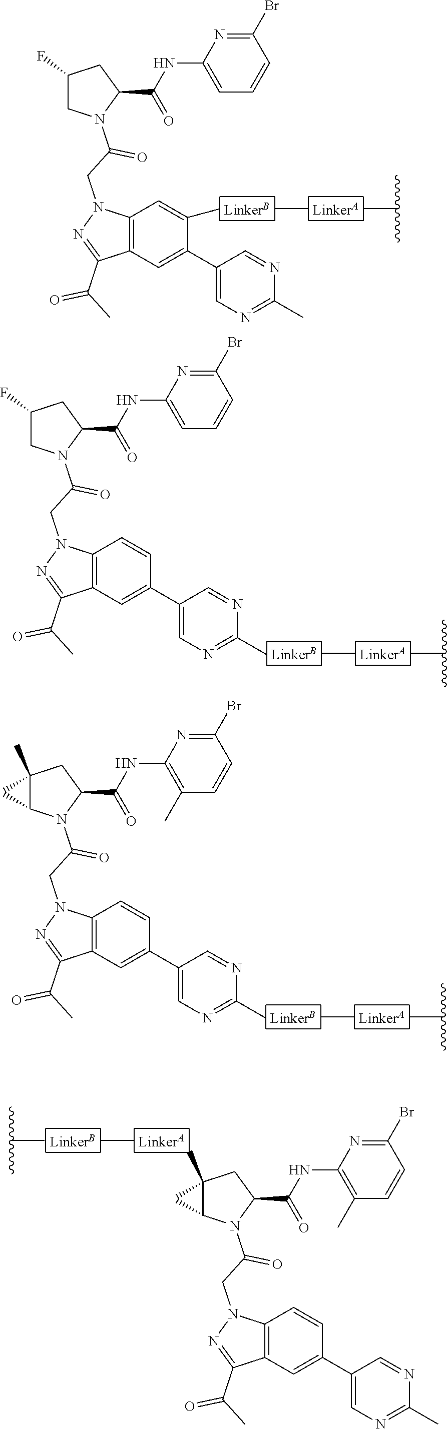

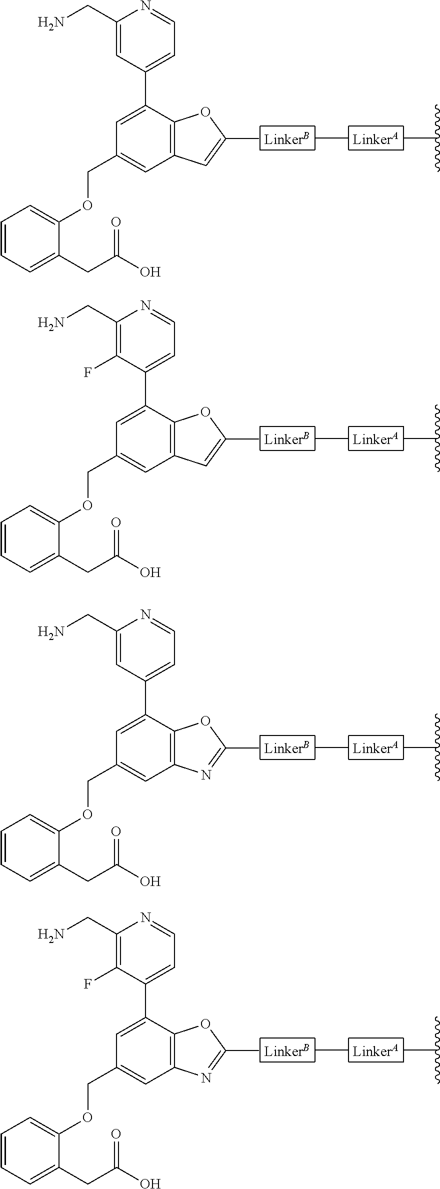

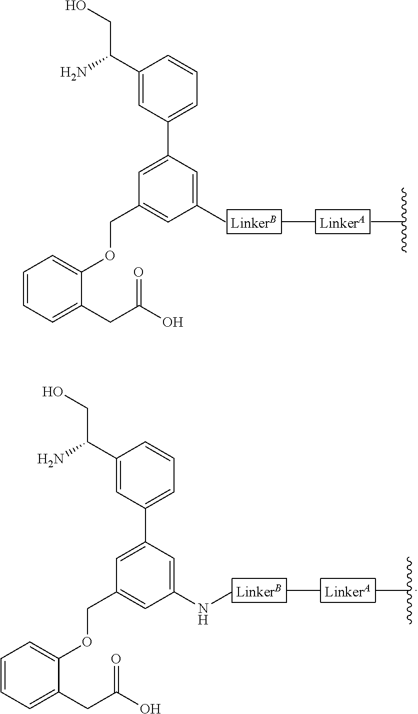

In certain embodiments the compound of the present invention is selected from the following compounds or a bi- or tri- dentate version thereof:

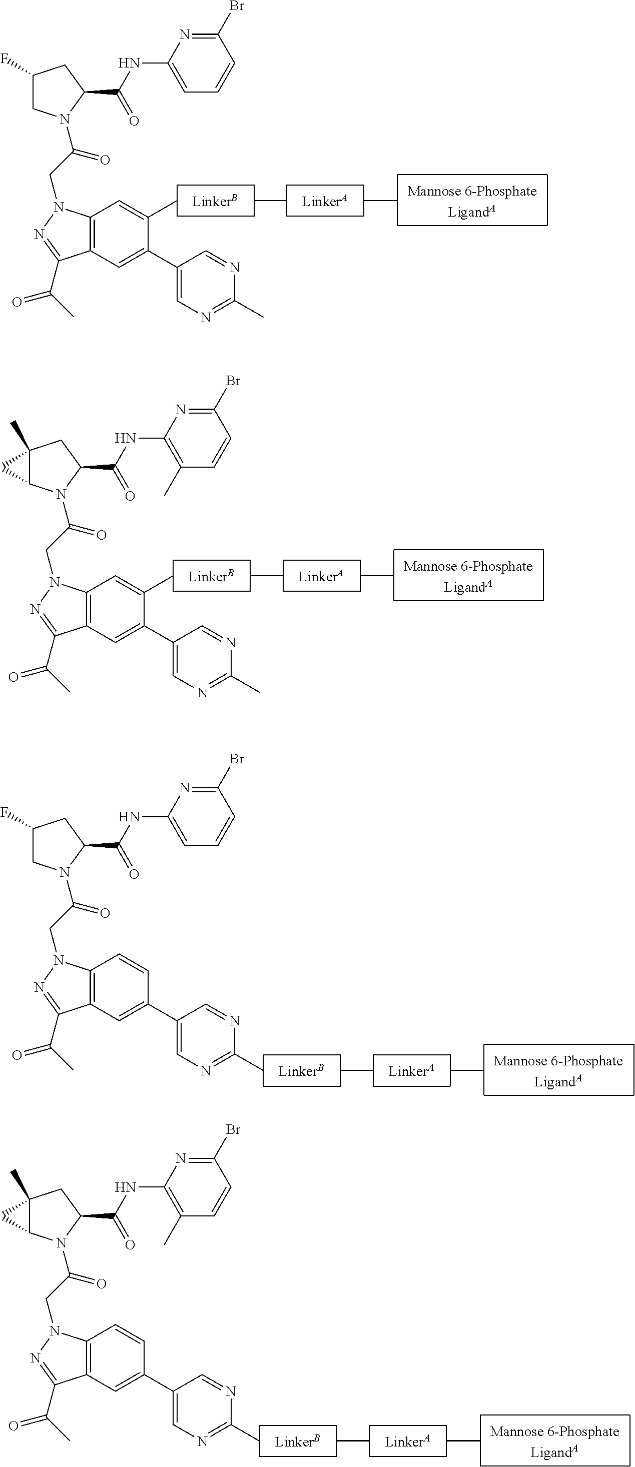

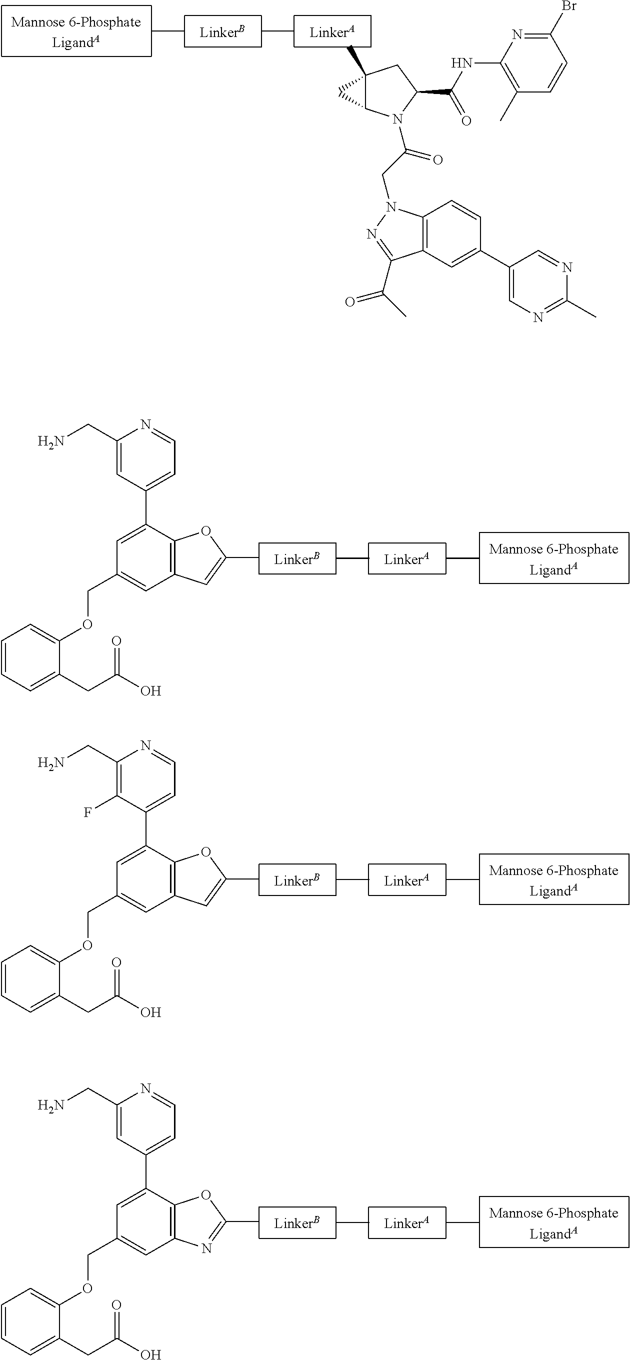

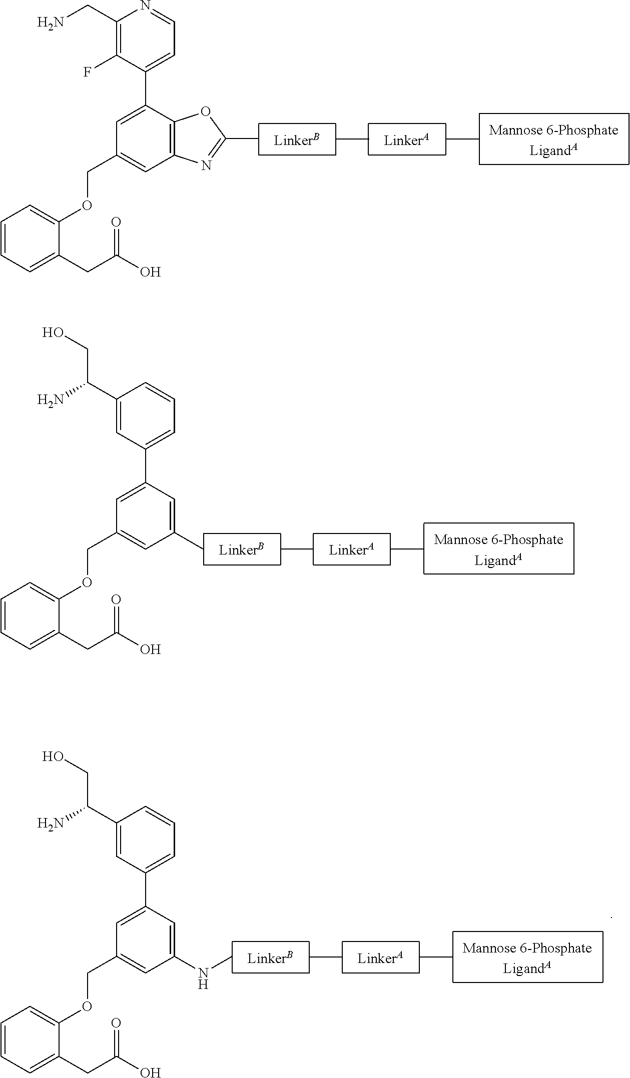

In certain embodiments the Factor D Targeting Ligand is selected from:

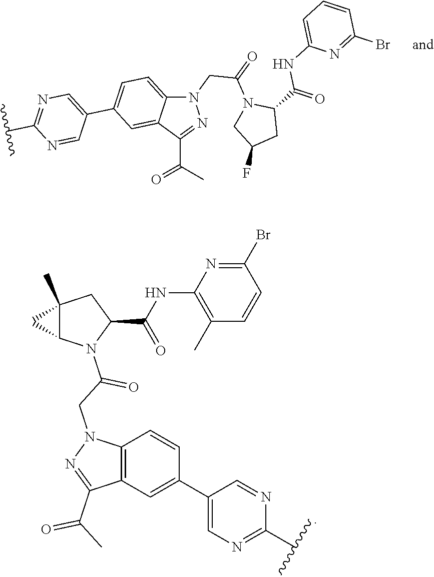 [SMILES: *c1ncc(-c2ccc3c(c2)c(C(C)=O)nn3CC(=O)N2C[C@H](F)C[C@H]2C(=O)Nc2cccc(Br)n2)cn1 ; "and" ; *c1ncc(-c2ccc3c(c2)c(C(C)=O)nn3CC(=O)N2[C@H](C(=O)Nc3nc(Br)ccc3C)C[C@@]3(C)C[C@@H]23)cn1]

### Factor H

In some embodiments, the Target Extracellular Protein is human complement factor H (UniProtKB-P08603 (CFAH_HUMAN)). Complement factor H is a glycoprotein that plays an essential role in maintaining a well-balanced immune response by modulating complement activation. Acts as a soluble inhibitor of complement, where its binding to self-markers such as glycan structures prevents complement activation and amplification on cell surfaces. Complement factor H accelerates the decay of the complement alternative pathway (AP) C3 convertase C3bBb, thus preventing local formation of more C3b, the central player of the complement amplification loop. As a cofactor of the serine protease factor I, CFH also regulates proteolytic degradation of already-deposited C3b. In addition, it mediates several cellular responses through interaction with specific receptors. For example, CFH interacts with CR3/ITGAM receptor and thereby mediates the adhesion of human neutrophils to different pathogens. In turn, these pathogens are phagocytosed and destroyed.

The Protein Data Bank website provides the crystal structure of highly similar mutants of complement factor H searchable by 3KXV and 3KZJ (Bhattacharjee, A., et al., Mol Immunol 2010, 47, 1686-1691); as well as the crystal structure of wild type complement factor H bound to various compounds searchable by 2UWN (Prosser, B. E., et al., J Exp Med 2007, 204, 2277); 5WTB (Zhang, Y., et al., Biochem J 2017, 474, 1619-1631); 5032 and 5035 (Xue, X., et al., Nat Struct Mol Biol 2017, 24, 643-651); 40NT (Blaum, B. S., et al., Nat Chem Biol 2015, 11, 77-82); and 4ZH1 (Blaum, B. S., et al., Glycobiology 2016, 26, 532-539).

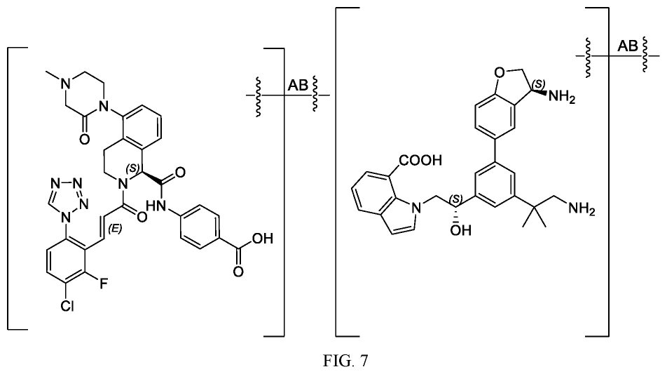

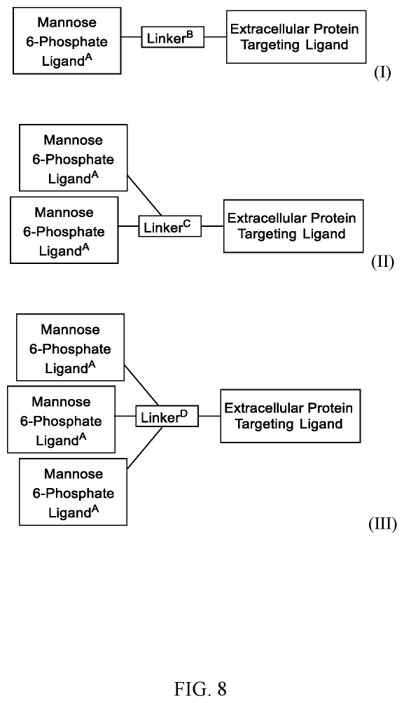

Representative complement factor H Targeting Ligands are provided in FIG. 7. Additional complement factor H Targeting Ligands are provided in, for example, J Immunol 182: 6394-6400 (2009), PLoS Pathogens 4: e1000250 (2008), PLoS Pathogens 6: e1001027 (2010), U.S. patent Ser. No. 10/865,238B1, U.S. Pat. No. 8,962,795B2, US patent application 20160317573A1, and US patent application 20190315842A1, each of which is incorporated by reference herein.

### Complement Component 5 (C5)

In some embodiments, the Target Extracellular Protein is human complement component 5 (C5) (UniProtKB-P01031 (CO5_HUMAN)). Activation of C5 by a C5 convertase initiates the spontaneous assembly of the late complement components, C5-C9, into the membrane attack complex. C5b has a transient binding site for C6. The C5b-C6 complex is the foundation upon which the lytic complex is assembled.

The Protein Data Bank website provides the crystal structure of Complement Component 5 searchable by 3CU7 (Fredslund, F., Nat Immunol 2008, 9, 753-760); as well as the crystal structure of Complement Component 5 bound to various compound searchable by 5I5K (Schatz-Jakobsen, J. A., et al, J Immunol 2016, 197, 337-344); 3PVM and 3PRX (Laursen, N. S., et al., EMBO J 2011, 30, 606-616); and 3KLS (Laursen, N. S., et al., Proc Natl Acad Sci 2010, 107, 3681-3686).

Representative Complement Component 5 Targeting Ligands are provided in the figures. Additional Complement Component 5 Targeting Ligands are provided in, for example, J Immunol 197: 337-344 (2016), Ther Adv Hematol 10: 1-11 (2019), BioDrugs 34: 149-158 (2020), Blood 135: 884-885 (2020), US patent application 20170342139A1, and US patent application 20200095307A1, each of which is incorporated by reference herein.

In certain embodiments the Extracellular Targeting Ligand is selected from:

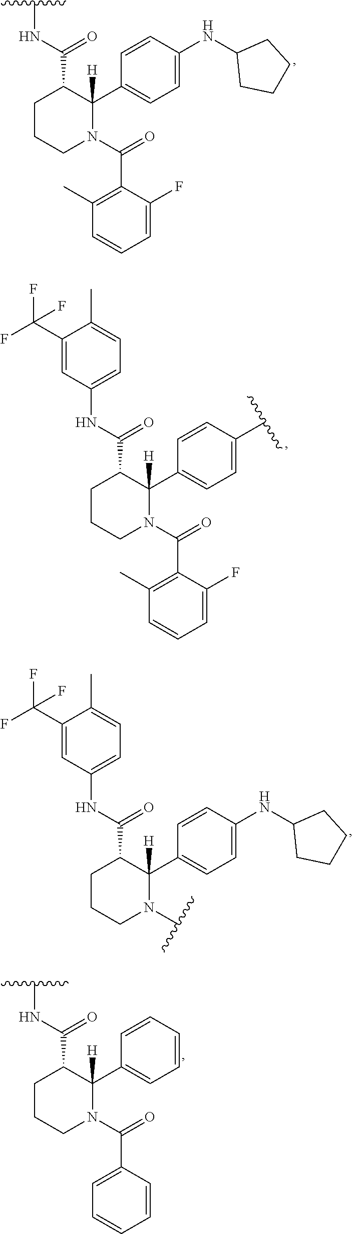 [SMILES: *NC(=O)[C@H]1CCCN(C(=O)c2c(C)cccc2F)[C@H]1c1ccc(NC2CCCC2)cc1 ; *c1ccc([C@H]2[C@@H](C(=O)Nc3ccc(C)c(C(F)(F)F)c3)CCCN2C(=O)c2c(C)cccc2F)cc1 ; *N1CCC[C@H](C(=O)Nc2ccc(C)c(C(F)(F)F)c2)[C@@H]1c1ccc(NC2CCCC2)cc1 ; *NC(=O)[C@H]1CCCN(C(=O)c2ccccc2)[C@H]1c1ccccc1]

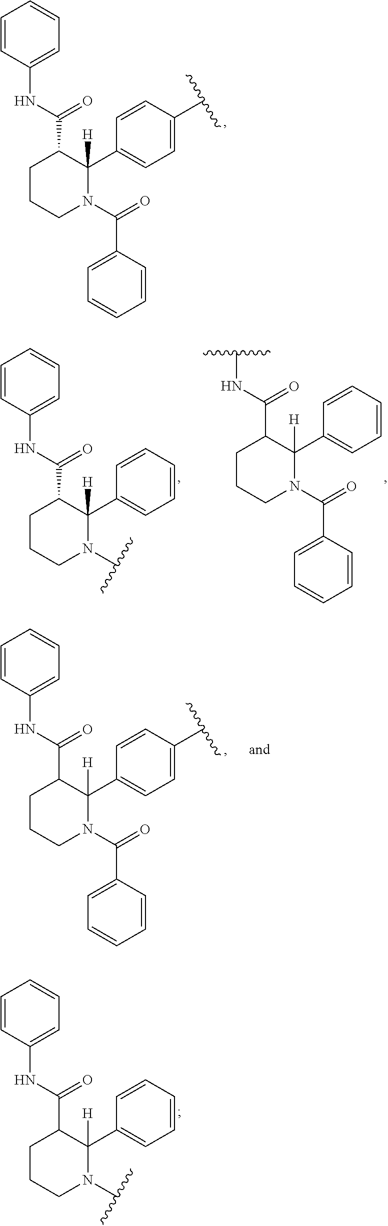 [SMILES: *c1ccc([C@H]2[C@@H](C(=O)Nc3ccccc3)CCCN2C(=O)c2ccccc2)cc1 ; *N1CCC[C@H](C(=O)Nc2ccccc2)[C@@H]1c1ccccc1 ; *NC(=O)C1CCCN(C(=O)c2ccccc2)C1c1ccccc1 ; *c1ccc(C2C(C(=O)Nc3ccccc3)CCCN2C(=O)c2ccccc2)cc1 ; *N1CCCC(C(=O)Nc2ccccc2)C1c1ccccc1]

each of which is optionally substituted with 1, 2, 3, or 4 substituents independently selected from R²¹.

In certain embodiments the complement C5 Targeting Ligand is selected from a ligand described in Jendza, K. et al. A Small-Molecule Inhibitor of C5 Complement Protein. Nat Chem Biol 2019, 15 (7), 666-668; or Zhang, M.; Yang, X.-Y.; Tang, W.; Groeneveld, T. W. L.; He, P.-L.; Zhu, F.-H.; Li, J.; Lu, W.; Blom, A. M.; Zuo, J.-P.; Nan, F.-J. Discovery and Structural Modification of 1-Phenyl-3-(1-Phenylethyl)Urea Derivatives as Inhibitors of Complement. ACS Med. Chem. Lett. 2012, 3 (4), 317-321.

In certain embodiments the C5 Targeting Ligand is selected from:

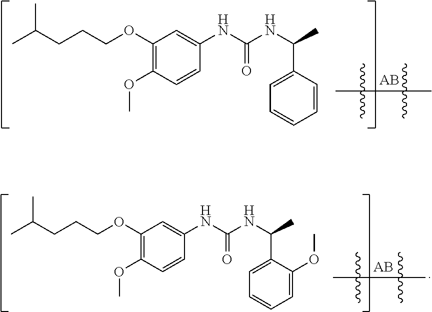

In certain embodiments the C5 Targeting Ligand is selected from:

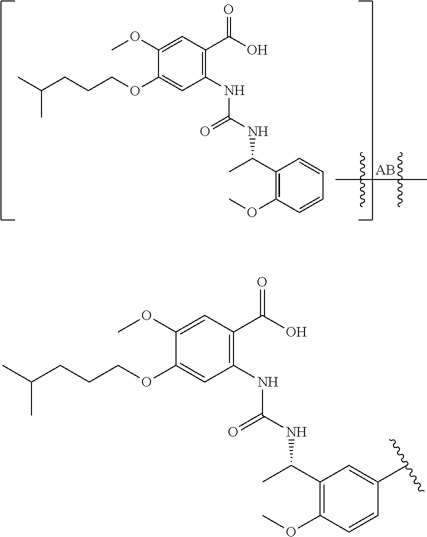

### Complement C1s

In certain embodiments the extracellular targeting ligand is a C1s Targeting Ligand.

In certain embodiments the complement C1s Targeting Ligand is selected from a ligand described in WO2020/198062 or U.S. Pat. No. 6,683,055.

In certain embodiments the compound of the present invention is selected from the following compounds or a bi- or tri- dentate version thereof:

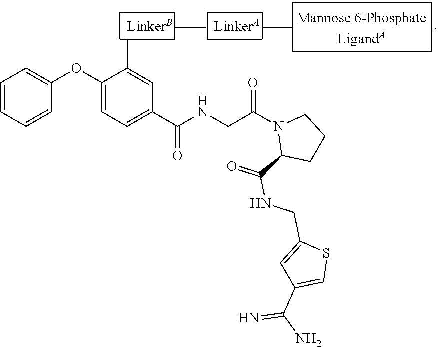 [SMILES: Cc1cc(C(=O)NCC(=O)N2CCC[C@H]2C(=O)NCc2cc(C(=N)N)cs2)ccc1Oc1ccccc1]

### MASP

In certain embodiments the extracellular targeting ligand is a MASP Targeting Ligand.

In certain embodiments the MASP Targeting Ligand is selected from a ligand described in Héja, D. et al. Monospecific Inhibitors Show That Both Mannan-Binding Lectin-Associated Serine Protease-1 (MASP-1) and -2 Are Essential for Lectin Pathway Activation and Reveal Structural Plasticity of MASP-2. Journal of Biological Chemistry 2012, 287 (24), 20290-20300; Dobó, J.; Kocsis, A.; Gil, P. Be on Target: Strategies of Targeting Alternative and Lectin Pathway Components in Complement-Mediated Diseases. Front. Immunol. 2018, 9, 1851; or WO 2014/144542.

In certain embodiments the MSAP-1 Targeting Ligand is SGMI-1 peptide, linked through the N- or C-terminus.

In certain embodiments the MSAP-1 Targeting Ligand is SGMI-2 peptide, linked through the N- or C-terminus.

In certain embodiments the MSAP-1 Targeting Ligand is TFMI-3 peptide, linked through the N- or C-terminus.

### Factor XIa

In certain embodiments the extracellular targeting ligand is a factor XIa Targeting Ligand.

In certain embodiments the factor XIa Targeting Ligand is selected from a ligand described in: Lorthiois, E. et al. Structure-Based Design and Preclinical Characterization of Selective and Orally Bioavailable Factor XIa Inhibitors: Demonstrating the Power of an Integrated S1 Protease Family Approach. J. Med. Chem. 2020, 63 (15), 8088-8113.

In certain embodiments the factor XIa Targeting Ligand is selected from a ligand described in: Quan, M. L. et al. Factor XIa Inhibitors as New Anticoagulants. J. Med. Chem. 2018, 61 (17), 7425-7447.

In certain embodiments the factor XIa Targeting Ligand is selected from a ligand described in: Yang, W. et al. Discovery of a High Affinity, Orally Bioavailable Macrocyclic FXIa Inhibitor with Antithrombotic Activity in Preclinical Species. J. Med. Chem. 2020, 63 (13), 7226-7242.

In certain embodiments the factor XIa Targeting Ligand-Linker is:

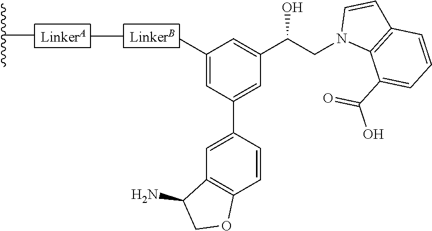

In certain embodiments the compound of the present invention is selected from the following compounds or a bi- or tri- dentate version thereof:

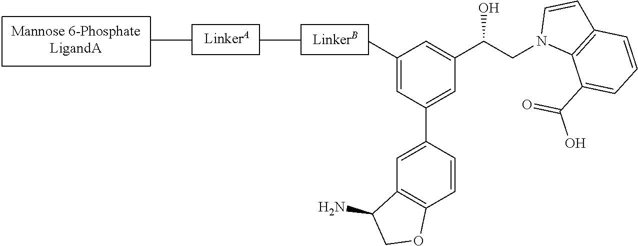 [SMILES: Cc1cc(-c2ccc3c(c2)[C@H](N)CO3)cc([C@H](O)Cn2ccc3cccc(C(=O)O)c32)c1]

In certain embodiments the factor Xia Targeting Ligand is selected where an anchor bond is placed at any suitable location with or without functionalization.

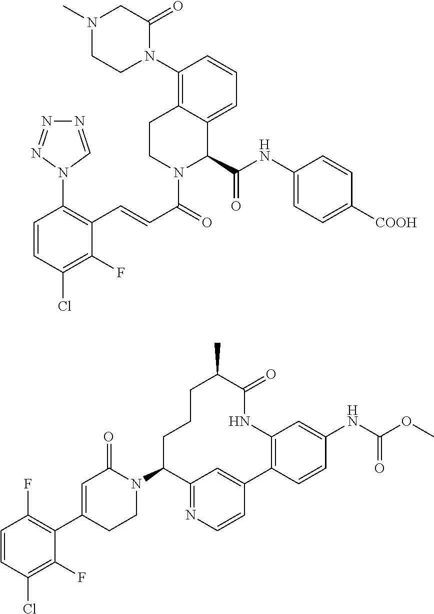 [SMILES: CN1CCN(c2cccc3c2CCN(C(=O)/C=C/c2c(-n4cnnn4)ccc(Cl)c2F)[C@@H]3C(=O)Nc2ccc(C(=O)O)cc2)C(=O)C1 ; COC(=O)Nc1ccc2c(c1)NC(=O)[C@H](C)CCC[C@H](N1CCC(c3c(F)ccc(Cl)c3F)=CC1=O)c1cc-2ccn1]

In certain embodiments the Factor XIa Targeting Ligand is selected from:

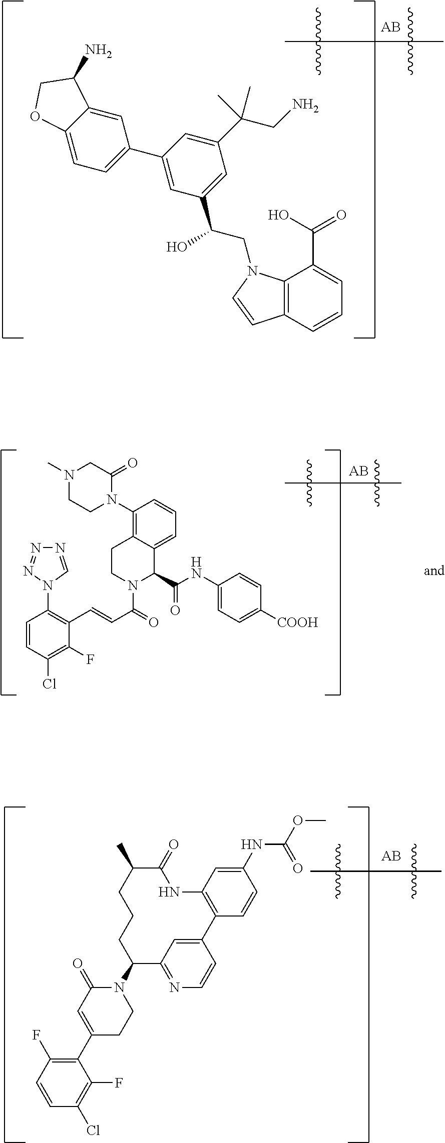

### Immunoglobulin Degradation

Immunoglobulins, for example IgG, can cause, modulate, or amplify diseases in vivo, such as abnormal cellular proliferation such as tumors and cancer, autoimmune disorders, inflammation, and aging-related diseases. For example, immunoglobulins bind to cell surface receptors, often initiating aberrant signaling in multiple diseases such as cancer and inflammation.

The immunoglobulin degraders described herein or their pharmaceutically acceptable salt and/or pharmaceutically acceptable compositions thereof can be used to treat a disorder which is mediated by an immunoglobulin that binds to the Immunoglobulin Targeting Ligand. The described degraders are capable of targeting immunoglobulins that mediate pathological disorders for lysosomal degradation. The selected immunoglobulin may modulate a disorder in a human via a mechanism of action such as modification of a biological pathway, pathogenic signaling, or modulation of a signal cascade or cellular entry. The immunoglobulin is recruited with an Immunoglobulin Targeting Ligand, which is a ligand for the immunoglobulin.

Accordingly, in some embodiments, a method to treat a host with a disorder mediated by an immunoglobulin is provided that includes administering an effective amount of a degrader targeting the immunoglobulin or its pharmaceutically acceptable salt described herein to the host, typically a human, optionally in a pharmaceutically acceptable composition.

The immunoglobulin can be either the normal form of the protein or an aberrant form. For example, the immunoglobulin can be a mutant protein, or a protein, for example, where a partial, or full, gain-of-function or loss-of-function is encoded by nucleotide polymorphisms.

Targeting specific immunoglobulins is accomplished by the present invention through the use of specific Immunoglobulin Targeting Ligands. The target immunoglobulins of the current invention may include, but are not limited to, immunoglobulin G (IgG), immunoglobulin A (IgA), and immunoglobulin E (IgE). These immunoglobulins mediate a range of diseases that can be treated with an effective amount of the disclosed Immunoglobulin Degraders described herein.

### Immunoglobulin A (IgA)

Aberrant expression of immunoglobulin A (IgA) mediates a range of autoimmune and immune-mediated disorders, including IgA nephropathy (also known as Berger's disease), celiac disease, Crohn's disease, Henoch-Schonlein purpura (HSP) (also known as IgA vasculitis), IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, α-chain disease, IgA monoclonal gammopathy, monoclonal gammopathy of undetermined significance (MGUS), linear IgA bullous dermatosis, rheumatoid arthritis, ulcerative colitis, and primary glomerulonephritis, among others.

Specific degradation of IgA can be accomplished through the use of an IgA-specific Immunoglobulin Targeting Ligand. In certain embodiments, the Immunoglobulin Targeting Ligand used is an Opt peptide. Variations and derivatives of the IgA-specific Opt peptide suitable for use as IgA-specific Immunoglobulin Targeting Ligands are described in Hatanaka et al. Journal of Biological Chemistry, 287(51) 43126-43136. In certain embodiments, the IgA-specific Immunoglobulin Targeting Ligand is Opt-1. In certain embodiments, the IgA-specific Immunoglobulin Targeting Ligand is Opt-2. In certain embodiments, the IgA-specific Immunoglobulin Targeting Ligand is Opt-3.

In certain embodiments the immunoglobulin degrading compound is:

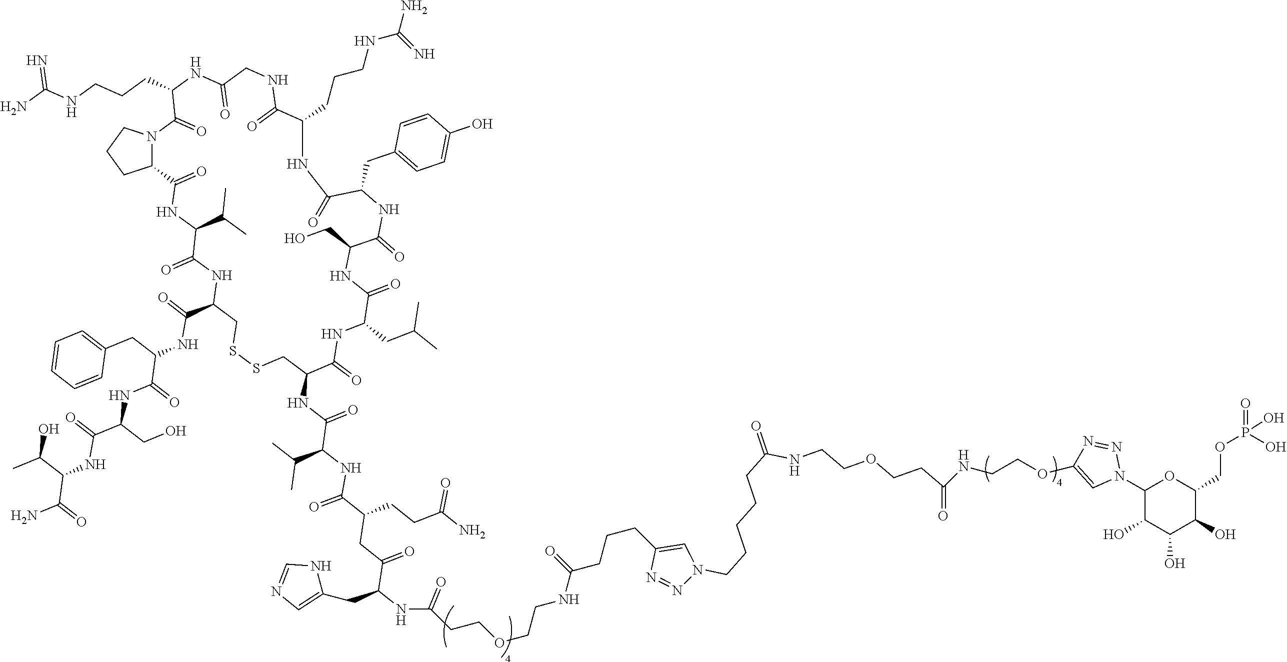

or a pharmaceutically acceptable salt thereof.

In certain embodiments the immunoglobulin degrading compound is:

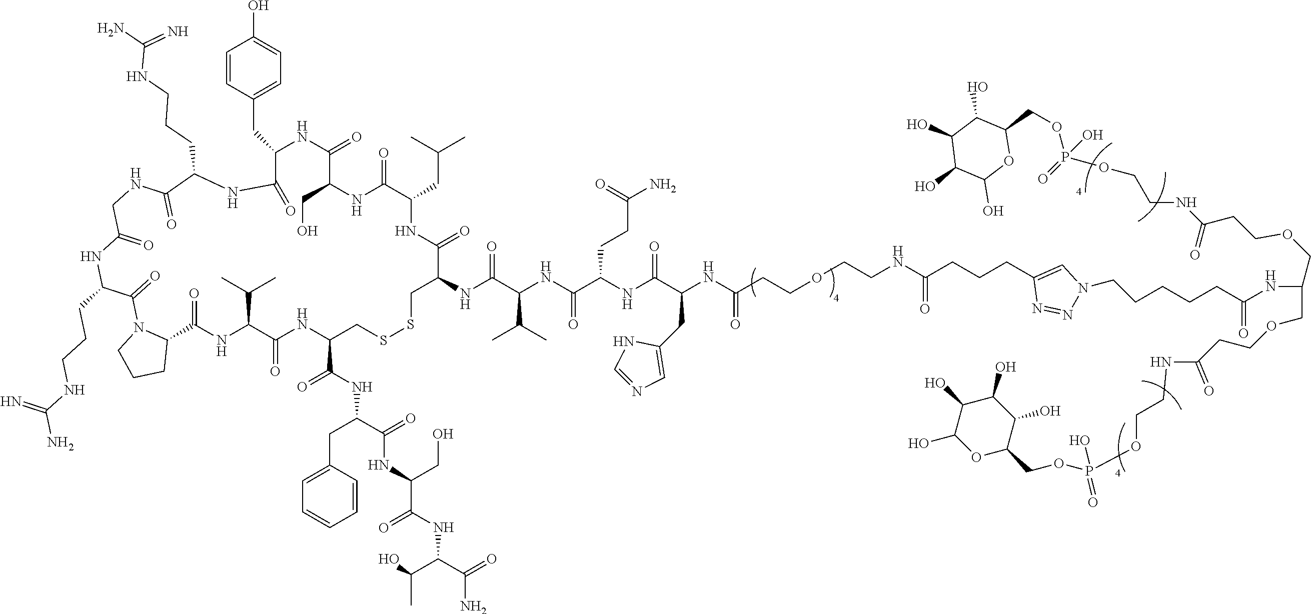

or a pharmaceutically acceptable salt thereof.

In certain embodiments the immunoglobulin degrading compound is:

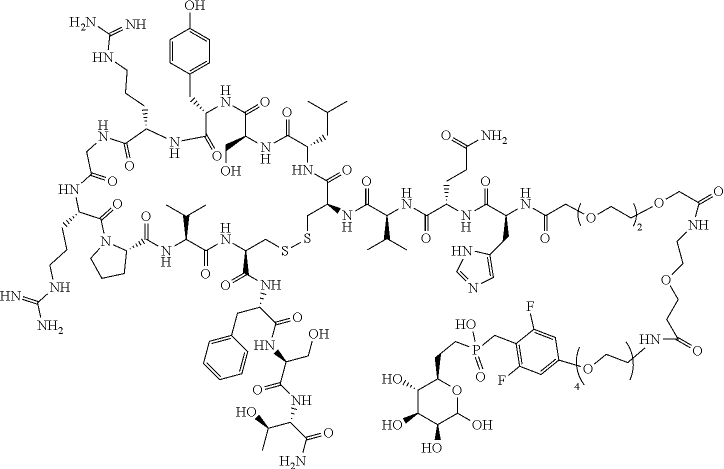

or a pharmaceutically acceptable salt thereof.

In certain embodiments the Immunoglobulin Targeting Ligand is:

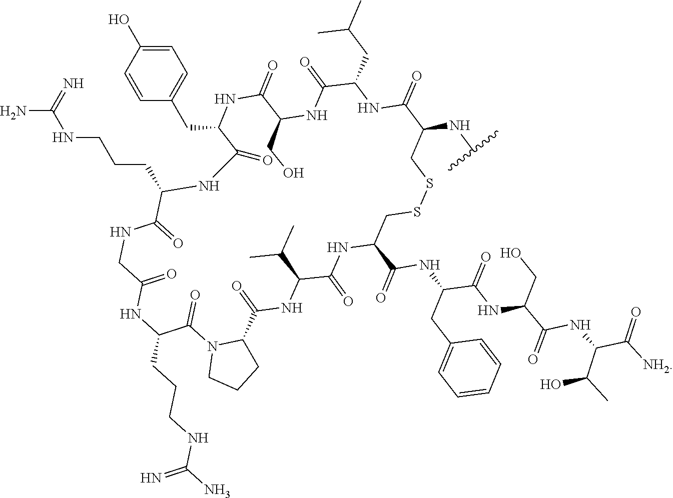 [SMILES: *N[C@H]1CSSC[C@@H](C(=O)N[C@@H](Cc2ccccc2)C(=O)N[C@@H](CO)C(=O)N[C@H](C(N)=O)[C@@H](C)O)NC(=O)[C@H](C(C)C)NC(=O)[C@@H]2CCCN2C(=O)[C@H](CCCNC(=N)N)NC(=O)CNC(=O)[C@H](CCCNC(=N)N)NC(=O)[C@H](Cc2ccc(O)cc2)NC(=O)[C@H](CO)NC(=O)[C@H](CC(C)C)NC1=O]

The Protein Data Bank website provides the crystal structure of IgA, as well as the crystal structure of IgA bound to various compounds searchable by 5E8E (Baglin, T. P., et al., J. Thromb. Haemost., 2016, 14: 137-142), and 2QTJ (Bonner, A., et al., J. Immunol., 2008, 180: 1008-1018). Additionally, Hatanaka T. et al., provides great insight into the specificity and high binding affinity of IgA to OPT-1 peptides (J Biol Chem., 2012, 287(51), 43126-43136.).

Representative IgA Targeting Ligands are provided in FIG. 1.

Additional representative Targeting Ligands include:

SEQ ID NO: 1
MLKKIE
(Jerlstrom et al. Infect. Immun. 1996 Jul;
64(7):2787-2793;
SEQ ID NO: 2
VEPNCDIHVMWEWECFERL
(Biochemistry 1998, 37, 17754-177764)
SEQ ID NO: 3
TVFTSWEEYLDWV
(J. Bio. Chem. 2014 Jan; 289(2):942-955)
SEQ ID NO: 4
*(Ac)-FVPTTX(N-Me)AX(N-Me)AEAPC*
SEQ ID NO: 5
*(Ac)-FVDTTS(N-Me)FX(N-Me)ENSPC*
SEQ ID NO: 6
*(Ac)-FVSTTX(N-Me)AX(N-Me)ADRPC*
SEQ ID NO: 7
*(Ac)-FVDTTS(N-Me)FX(N-Me)ANSPC*
SEQ ID NO: 8
*(Ac)-FVDSTT(N-Me)AX(N-Me)ANHPC*
SEQ ID NO: 9
*(Ac)-FVDTTS(N-Me)FX(N-Me)AESPC*
SEQ ID NO: 10
*(Ac)-FVDTTS(N-Me)F(4CF)F(N-Me)AESPC*
SEQ ID NO: 11
*(Ac)-FVDTTS(N-Me)AX(N-Me)AKSPC*
SEQ ID NO: 12
*(Ac)-FVSTTX(N-Me)AX(N-Me)ADSPC*
SEQ ID NO: 13
*(Ac)-FVSTTS(N-Me)FX(N-Me)ADRPC*
SEQ ID NO: 14
*(Ac)-FVDTTX(N-Me)AX(N-Me)AESPC*
SEQ ID NO: 15
*(Ac)-FVSTT(4CF3)F(N-Me)A(4CF3)F(N-Me)AERPC*
SEQ ID NO: 16
*(Ac)-FVSTTS(N-Me)FX(N-Me)AESPC*
SEQ ID NO: 17
*(Ac)-FVSTTX(N-Me)FX(N-Me)AESPC*
SEQ ID NO: 18
*(Ac)-FVSTTX(N-Me)A(3,4diCI)F(N-Me)ADRPC*
SEQ ID NO: 19
*(Ac)-FVSTTX(N-Me)A(3FI)F(N-Me)ADRPC*
SEQ ID NO: 20
*(Ac)-FVDTTA(N-Me)FX(N-Me)AEAPC*
SEQ ID NO: 21
*(Ac)-FVDTTF(N-Me)AX(N-Me)AESPC*
SEQ ID NO: 22
*(Ac)-FVDTTA(N-Me)FX(N-Me)AQAPC*
SEQ ID NO: 23
*(Ac)-FVPTTX(N-Me)AX(N-Me)ADRPC*
SEQ ID NO: 24
*(Ac)-FVSTTX(N-Me)AX(N-Me)APSPC*
SEQ ID NO: 25
*(Ac)-FVPTTA(N-Me)FX(N-Me)AEAPC*
SEQ ID NO: 26
*(Ac)-FVATTF(N-Me)AX(N-Me)AKAPC*
SEQ ID NO: 27
*(Ac)-FVNTTF(N-Me)AX(N-Me)AKAPC*
SEQ ID NO: 28
*(Ac)-FVSTTF(N-Me)AX(N-Me)AEAPC*
SEQ ID NO: 29
*(Ac)-FVSTTX(N-Me)AX(N-Me)AESPC*
SEQ ID NO: 30
*(Ac)-FVDTTX(N-Me)A(3,4diCI)F(N-Me)AESPC*
SEQ ID NO: 31
*(Ac)-FENTTF(N-Me)AX(N-Me)AASPC*
SEQ ID NO: 32
*(Ac)-FVPTTF(N-Me)A(4CI)F(N-Me)APAPC*
SEQ ID NO: 33
*(Ac)-FVPTTF(N-Me)A(4CI)F(N-Me)ADAPC*
SEQ ID NO: 34
*(Ac)-FV-Hse-TTF(N-Me)A(4CI)F(N-Me)ADAPC*
SEQ ID NO: 35
*(Ac)-FVATTF(N-Me)A(4CI)F(N-Me)ANAPC*
SEQ ID NO: 36
*(Ac)-FVPTTV(N-Me)A(4CI)F(N-Me)AEAPC*
SEQ ID NO: 37
*(Ac)-FVNTTF(N-Me)AX(N-Me)A(N-Me)EAPC*
SEQ ID NO: 38
*(Ac)-FVPTTF(N-Me)AX(N-Me)AEAPC*
SEQ ID NO: 39
*(Ac)-FVPTTX(N-Me)AX(N-Me)AAAPC*
SEQ ID NO: 40
Opt-1-HMVCLAYRGRPVCFAL
(Hatanaka et al. J. Biol. Chem. Vol. 287,
No. 51, pp. 43126-43136, Dec. 14, 2012)
SEQ ID NO: 41
Opt-2-HMVCLSYRGRPVCFSL
(Hatanaka et al. J. Biol. Chem. Vol. 287, No.
51, pp. 43126-43136, Dec. 14, 2012)
SEQ ID NO: 42
Opt-3-HQVCLSYRGRPVCFST
(Hatanaka et al. J. Biol. Chem. Vol. 287,
No. 51, pp. 43126-43136, Dec. 14, 2012)
SEQ ID NO: 43
QMRCLSYKGRRVCLWL
(U.S. Pat. No. 9,593,147)
SEQ ID NO: 44
KRLCLQYKGSKVCFRL
(U.S. Pat. No. 9593 147)
SEQ ID NO: 45
RMRCLTYRGRRVCLEL
(U.S. Pat. No. 9,593,147)
SEQ ID NO: 46
SMRCLQYRGSRVCLTL
(U.S. Pat. No. 9,593,147)
SEQ ID NO: 47
HLRCLRYKGTRVCFSL
(U.S. Pat. No. 9,593,147)
SEQ ID NO: 48
HVRCLSYKGREVCVQL
(U.S. Pat. No. 9,593,147)
SEQ ID NO: 49
PRMCLFIYKGRRVCIPY
(U.S. Pat. No. 9,593,147)
SEQ ID NO: 50
HMRCLHYKGRRVCFLL
(U.S. Pat. No. 9,593,147)
SEQ ID NO: 51
HKRCLHYRGRMVCFLI
(U.S. Pat. No. 9,593,147)
SEQ ID NO: 52
QKRCLKYKGSRVCFFL
(U.S. Pat. No. 9,593,147)
SEQ ID NO: 53
HVRCLRYRGKNVCFLL
(U.S. Pat. No. 9,593,147)
SEQ ID NO: 54
SDVCLRYRGRPVCFQV
(U.S. Pat. No. 9,593,147)
SEQ ID NO: 55
RDVCLRYRGRPVCFQV
(U.S. Pat. No. 9,593,147)
SEQ ID NO: 56
HDVCLRYRGRPVCFQV
(U.S. Pat. No. 9,593,147)
SEQ ID NO: 57
SMVCLRYRGRPVCFQV
(U.S. Pat. No. 9,593,147)
SEQ ID NO: 58
SAVCLRYRGRPVCFQV
(U.S. Pat. No. 9,593,147)
SEQ ID NO: 59
SDVCLNYRGRPVCFQV
(U.S. Pat. No. 9,593,147)
SEQ ID NO: 60
SDVCLHYRGRPVCFQV
(U.S. Pat. No. 9,593,147)
SEQ ID NO: 61
SDVCLAYRGRPVCFQV
(U.S. Pat. No. 9,593,147)
SEQ ID NO: 62
SDVCLRYRGRPVCFAV
(U.S. Pat. No. 9,593,147)
SEQ ID NO: 63
SDVCLRYRGRPVCFQL
(U.S. Pat. No. 9,593,147)
SEQ ID NO: 64
SDVCLRYRGRPVCFQA
(U.S. Pat. No. 9,593,147)
SEQ ID NO: 65
HMVCLSYRGRPVCF
(US Pub. No. 20150044701)
SEQ ID NO: 66
HMVCLSYRGRPVCFS
(US Pub. No. 20150044701)
SEQ ID NO: 67
HQVCLSYRGQPVCFSL
(US Pub. No. 20150044701)
SEQ ID NO: 68
HQVCLSYRGRPTCFSL
(US Pub. No. 20150044701)
SEQ ID NO: 69
HQVCLSYRGRPVCYSL
(US Pub. No. 20150044701)
SEQ ID NO: 70
HQVCLSYRGQPVCFST
(US Pub. No. 20150044701)
SEQ ID NO: 71
HQVCLSYRGRPTCFST
(US Pub. No. 20150044701)
SEQ ID NO: 72
HQVCLSYRGQPTCFST
(US Pub. No. 20150044701)

In certain embodiments the IgA Targeting Ligand is

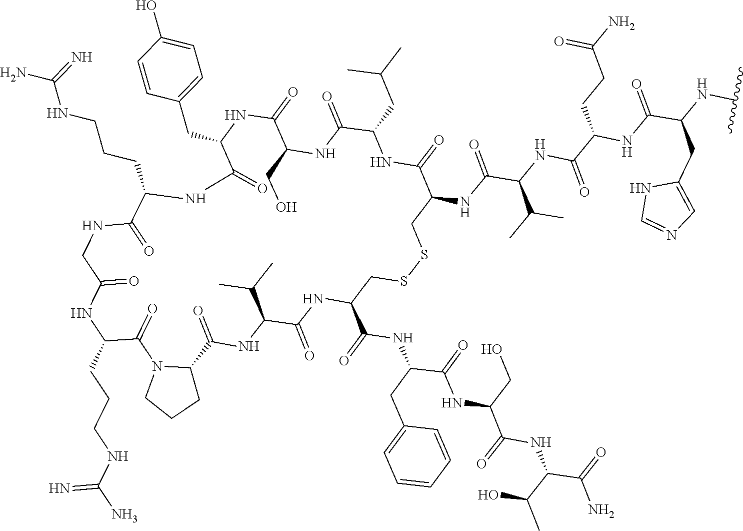 [SMILES: *N[C@@H](Cc1cnc[nH]1)C(=O)N[C@@H](CCC(N)=O)C(=O)N[C@H](C(=O)N[C@H]1CSSC[C@@H](C(=O)N[C@@H](Cc2ccccc2)C(=O)N[C@@H](CO)C(=O)N[C@H](C(N)=O)[C@@H](C)O)NC(=O)[C@H](C(C)C)NC(=O)[C@@H]2CCCN2C(=O)[C@H](CCCNC(=N)N)NC(=O)CNC(=O)[C@H](CCCNC(=N)N)NC(=O)[C@H](Cc2ccc(O)cc2)NC(=O)[C@H](CO)NC(=O)[C@H](CC(C)C)NC1=O)C(C)C]

### Immunoglobulin G (IgG)

Immunoglobulin G (IgG) mediates a range of autoimmune, infectious and metabolic diseases, including systemic fibroinflammatory disease. In addition, overexpression of IgG4 is associated with IgG4-related diseases, which generally include multiple organs, and disorders include type 1 autoimmune pancreatitis, interstitial nephritis, Riedel's thyroiditis, storiform fibrosis, Mikulicz's disease, Küttner's tumor, inflammatory pseudotumors (in various sites of the body), mediastinal fibrosis, retroperitoneal fibrosis (Ormond's disease), aortitis and periaortitis, proximal biliary strictures, idiopathic hypocomplementemic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenia gravis, ankylosing spondylitis, primary Sjögren's syndrome, psoriatic arthritis, systemic lupus erythematosus (SLE), sclerosing cholangitis, IgG monoclonal gammopathy, monoclonal gammopathy of undetermined significance (MGUS), melanoma, bullous pemphigoid, Goodpasture disease, encephalitis, thrombotic thrombocytopenic purpura, chronic inflammatory polyneuropathy, limbic encephalitis, neuromyotonia, Morvan syndrome, pemphigus foliaceus, pemphigus vulgaris, REM and non-REM parasomnia, and membranous nephropathy, multiple sclerosis, hyperthyroid Grave's disease, epidermolysis bullosa acquisita, pemphigoid gestationis, anti-p200 pemphigoid, and paraneoplastic pemphigus, among others.

Specific degradation of IgG can be accomplished through the use of an IgG-specific Immunoglobulin Targeting Ligand. In certain embodiments, the Immunoglobulin Targeting Ligand binds to the Fc region of IgG. In certain embodiments the IgG-specific Immunoglobulin Targeting Ligand is an Fc-binding peptide. In certain embodiments, the IgG-specific Immunoglobulin Targeting Ligand is Fc-BP2. In certain embodiments, the IgG-specific Immunoglobulin Targeting Ligand is Fc-III.

In certain embodiments the Immunoglobulin Targeting Ligand is:

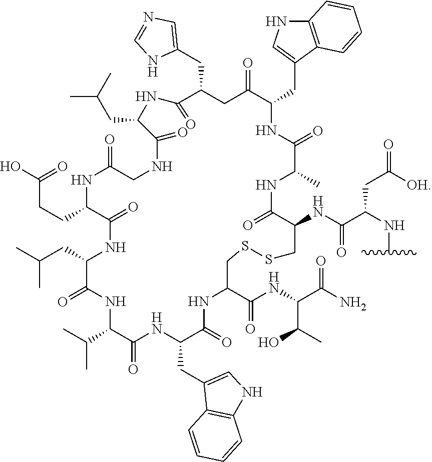 [SMILES: *N[C@@H](CC(=O)O)C(=O)N[C@H]1CSSCC(C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)CC(=O)[C@H](Cc2c[nH]c3ccccc23)NC(=O)[C@H](C)NC1=O]

In certain embodiments the Immunoglobulin Targeting Ligand is:

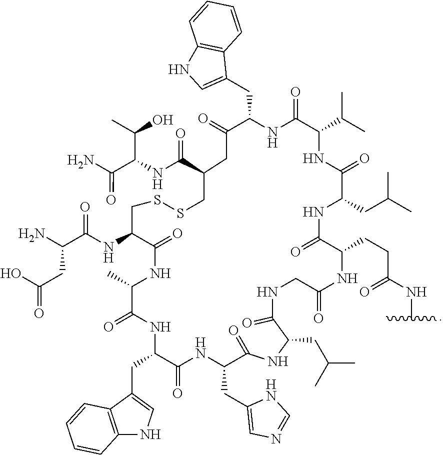 [SMILES: *NC(=O)CC[C@@H]1NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC(=O)[C@@H](NC(=O)[C@@H](N)CC(=O)O)CSSC[C@@H](C(=O)N[C@H](C(N)=O)[C@@H](C)O)CC(=O)[C@H](Cc2c[nH]c3ccccc23)NC(=O)[C@H](C(C)C)NC(=O)[C@H](CC(C)C)NC1=O]

In certain embodiments the Immunoglobulin Targeting Ligand is:

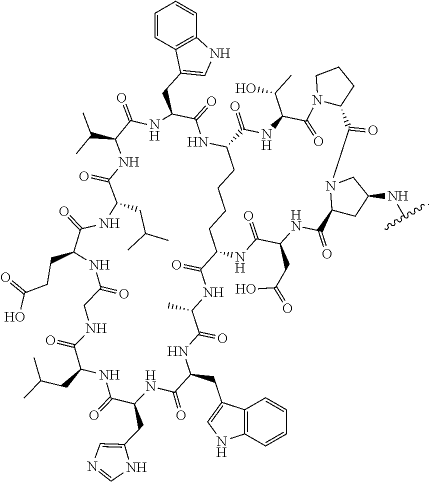 [SMILES: *N[C@H]1C[C@H]2C(=O)N[C@@H](CC(=O)O)C(=O)N[C@H]3CCCC[C@H](NC(=O)[C@H](Cc4c[nH]c5ccccc45)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc4cnc[nH]4)NC(=O)[C@H](Cc4c[nH]c5ccccc45)NC(=O)[C@H](C)NC3=O)C(=O)N[C@@H]([C@@H](C)O)C(=O)N3CCC[C@@H]3C(=O)N2C1]

The Protein Data Bank website provides the crystal structure of IgG searchable by 1H3X (Krapp, S., et al., J. Mol. Biol., 2003, 325: 979); and 5V43 (Lee, C. H., et al., Nat. Immunol., 2017, 18: 889-898); as well as the crystal structure of IgG bound to various compounds searchable by 5YC5 (Kiyoshi M., et al., Sci. Rep., 2018, 8: 3955-3955); 5XJE (Sakae Y., et al., Sci. Rep., 2017, 7: 13780-13780); 5GSQ (Chen, C. L., et al., ACS Chem. Biol., 2017, 12: 1335-1345); and 1HZH (Saphire E. O., et al., Science, 2001, 293: 1155-1159). Additionally, Kiyoshi, M., et al., provides insight into the structural basis for binding of human IgG1 to its high-affinity human receptor FcγRI. (Kiyosi M., et al., Nat Commun., 2015, 6, 6866).

Representative IgG Targeting Ligands are provided in FIG. 1.

Additional representative IgG Targeting Ligands include:

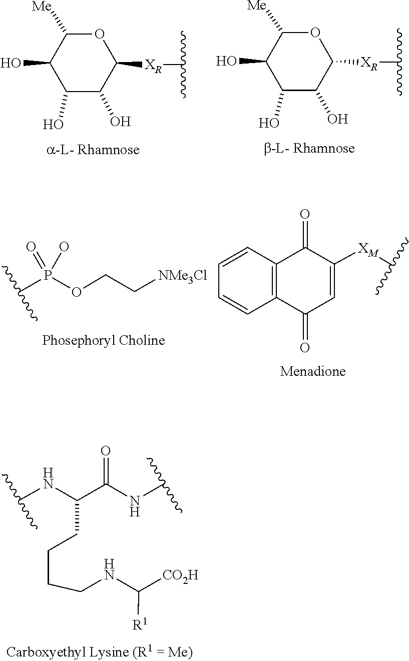

wherein X_{R} is O, S, NH, or N—C₁-C₃ alkyl; and
X_{M} is O, S, NH, or N—C₁-C₃ alkyl.

In other embodiments the IgG Targeting Ligand is selected from:

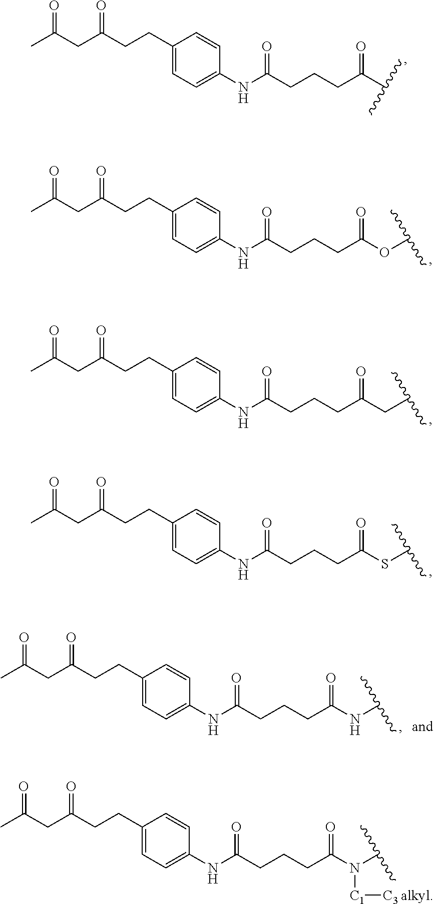

In some embodiments, the IgG Targeting Ligand is a group according to the chemical structure:

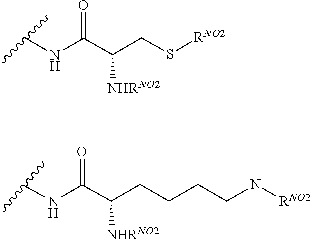

wherein R^{N02} is a dinitrophenyl group optionally linked through CH₂, S(O), S(O)₂, —S(O)₂O, —OS(O)₂, or OS(O)₂O.

In certain embodiments the IgG Targeting Ligand is selected from:

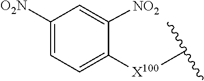

wherein X¹⁰⁰ is selected from O, CH₂, NH, N—C₁-C₃ alkyl, NC(O)C₁-C₃ alkyl, S(O), S(O)₂, —S(O)₂O, —OS(O)₂, or OS(O)₂O.

In some embodiments, the IgG Targeting Ligand is a 3-indoleacetic acid group according to the chemical structure:

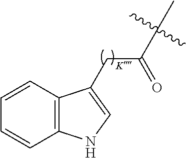

where k″″ is 1-4 (preferably 2-3, most often 3) or a

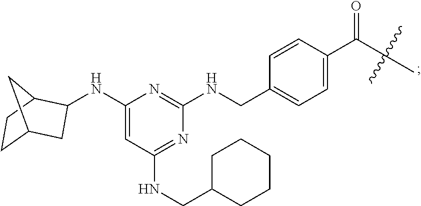 [SMILES: *C(=O)c1ccc(CNc2nc(NCC3CCCCC3)cc(NC3CC4CCC3C4)n2)cc1]

group.

In some embodiments, the IgG Targeting Ligand is a peptide. Nonlimiting examples of IgG Targeting Ligand peptides include:

SEQ ID NO: 73
PAM (RTY)₄K₂KG (Fassina, et al, J. Mol. Recognit. 1996, 9, 564-569)

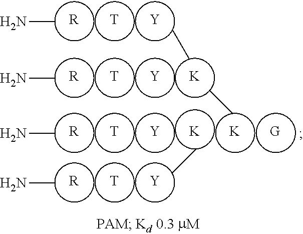

D-PAM, wherein the amino acids of the PAM sequence are all D-amino acids (Verdoliva, et al,
J. Immunol. Methods, 2002, 271, 77-88)
SEQ ID NO: 74
(RTY)₄K₂KG
D-PAM-Φ, wherein the amino acids of the PAM sequence are all D-amino acids with further
modifications wherein the four N-terminal arginines are acetylated with phenylactic acid (Dinon,
et al J. Mol. Recognit. 2011, 24, 1087-1094)
SEQ ID NO: 75
(RTY)₄K₂KG
SEQ ID NO: 76
TWKTSRISIF (Krook, et al,.J. Immunol. Methods 1998, 221, 151-157)
SEQ ID NO: 77
FGRLVSSIRY (Krook, et al, J. Immunol. Methods 1998, 221, 151-157)
SEQ ID NO: 78
Fc-III (DCAWHLGELVWCT-NH2) (DeLano et al, Science 2000, 287, 1279-1283)

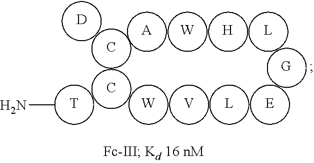

SEQ ID NO: 79
FCBP-Ser DSAWHLGELWST (see WO2014010813)
SEQ ID NO: 80
DCHKRSFWADNCT (see WO2014010813)
SEQ ID NO: 81
DCRTQFRPNQTCT (see WO2014010813)
SEQ ID NO: 82
DCQLCDFWRTRCT (see WO2014010813)
SEQ ID NO: 83
DCFEDFNEQRTCT (see WO2014010813)
SEQ ID NO: 84
DCLAKFLKGKDCT (see WO2014010813)
SEQ ID NO: 85
DCWHRRTHKTFCT (see WO2014010813)
SEQ ID NO: 86
DCRTIQTRSCT (see WO2014010813)
SEQ ID NO: 87
DCIKLAQLHSVCT (see WO2014010813)
SEQ ID NO: 88
DCWRHRNATEWCT (see WO2014010813)
SEQ ID NO: 89
DCQNWIKDVHKCT (see WO2014010813)
SEQ ID NO: 90
DCAWHLGELVWCT (see WO2014010813)
SEQ ID NO: 91
DCAFHLGELVWCT (see WO2014010813)
SEQ ID NO: 92
DCAYHLGELVWCT (see WO2014010813)
SEQ ID NO: 93
FcBP- 1 PAWHLGELVWP (Kang, et al, J. Chromatogr. A 2016, 1466, 105-112)

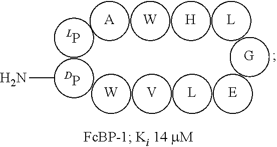

SEQ ID NO: 94
FcBP-2 PDCAWHLGELVWCTP (Dias, et al, J. Am. Chem. Soc. 2006, 128, 2726-2732)

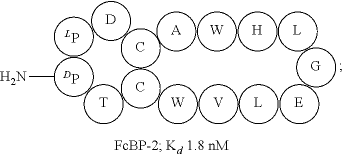

SEQ ID NO: 95
Fc-III-4c CDCAWHLGELVWCTC (Gong, et al, Bioconjug. Chem. 2016, 27, 1569-1573)

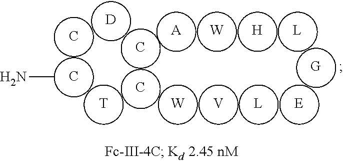

SEQ ID NO: 96
EPIHRSTLTALL (Ehrlich, et al, J. Biochem. Biophys. Method 2001, 49, 443-454)
SEQ ID NO: 97
APAR (Camperi, et al, Biotechnol. Lett. 2003, 25, 1545-1548)
SEQ ID NO: 98
FcRM (CFHH)2KG (Fc Receptor Mimetic, Verdoliva, et al., ChemBioChem 2005, 6, 1242-1253)

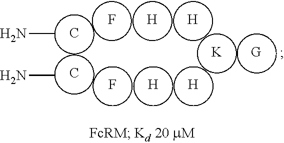

SEQ ID NO: 99
HWRGWV (Yang, et al., J Peptide Res. 2006, 66, 110-137)
SEQ ID NO: 100
HYFKFD (Yang, et al, J. Chromatogr. A 2009, 1216, 910-918)
SEQ ID NO: 101
HFRRHL (Menegatti, et al, J. Chromatogr. A 2016, 1445, 93-104)
SEQ ID NO: 102
HWCitGWV (Menegatti, et al, J. Chromatogr. A 2016, 1445, 93-104)
SEQ ID NO: 103
HWmetCitGWmetV (US10/266,566)
SEQ ID NO: 104
D₂AAG (Small Synthetic peptide ligand, Lund, et al, J. Chromatogr. A 2012, 1225, 158-167)
SEQ ID NO: 105
DAAG (Small Synthetic peptide ligand, Lund, et al, J. Chromatogr. A 2012, 1225, 158-167);
SEQ ID NO: 106
cyclo[(Nα-Ac) S(A)-RWHYFK-Lact-E] (Menegatti, et al, Anal. Chem. 2013, 85, 9229-9237);
SEQ ID NO: 107
cyclo[(Nα-Ac)-Dap(A)-RWHYFK-Lact-E] (Menegatti, et al, Anal. Chem. 2013, 85, 9229-9237);
SEQ ID NO: 108
cyclo[Link M-WFRHYK] (Menegatti, et al, Biotechnol. Bioeng. 2013, 110, 857-870);
SEQ ID NO: 109
NKFRGKYK (Sugita, et al, Biochem. Eng. J. 2013, 79, 33-40);
SEQ ID NO: 110
NARKFYKG (Sugita, et al, Biochem. Eng. J. 2013, 79, 33-40);
SEQ ID NO: 111
FYWHCLDE (Zhao, et al, Biochem. Eng. J. 2014, 88, 1-11);
SEQ ID NO: 112
FYCHWALE (Zhao, et al, J Chromatogr. A 2014, 1355, 107-114);
SEQ ID NO: 113
FYCHTIDE (Zhao, et al., Z Chromatogr. A 2014, 1359, 100-111);
SEQ ID NO: 114
Dual 1/3 (FYWHCLDE-FYCHTIDE) (Zhao, et al, J. Chromatogr. 1369, 64-72);
SEQ ID NO: 115
RRGW (Tsai, et al, Anal. Chem. 2014, 86, 293 1-2938);
SEQ ID NO: 116
KHRFNKD (Yoo and Choi, BioChip J. 2015, 10, 88-94);
SEQ ID NO: 117
CPSTHWK (Sun et al. Polymers 2018, 10, 778);
SEQ ID NO: 118
NVQYFAV (Sun et al. Polymers 2018, 10, 778);
SEQ ID NO: 119
ASHTQKS (Sun et al. Polymers 2018, 10, 778);
SEQ ID NO: 120
QPQMSHM (Sun et al. Polymers 2018, 10, 778);
SEQ ID NO: 121
TNIESLK (Sun et al. Polymers 2018, 10, 778);
SEQ ID NO: 122
NCHKCWN (Sun et al. Polymers 2018, 10, 778);
SEQ ID NO: 123
SHLSKNF (Sun et al. Polymers 2018, 10, 778).

In some embodiments the IgG Targeting Ligand is specific for IgG4.

In some embodiments the IgG4 specific Targeting Ligand is described in Gunnarsson et al. Biomolecular Engineering 2006, 23, 111-117.

In some embodiments the IgG4 specific targeting ligand is selected from

SEQ ID NO: 124
FDLLEHFY
and
SEQ ID NO: 125
DLLHHFDYF.

Additional IgG Targeting Ligands include

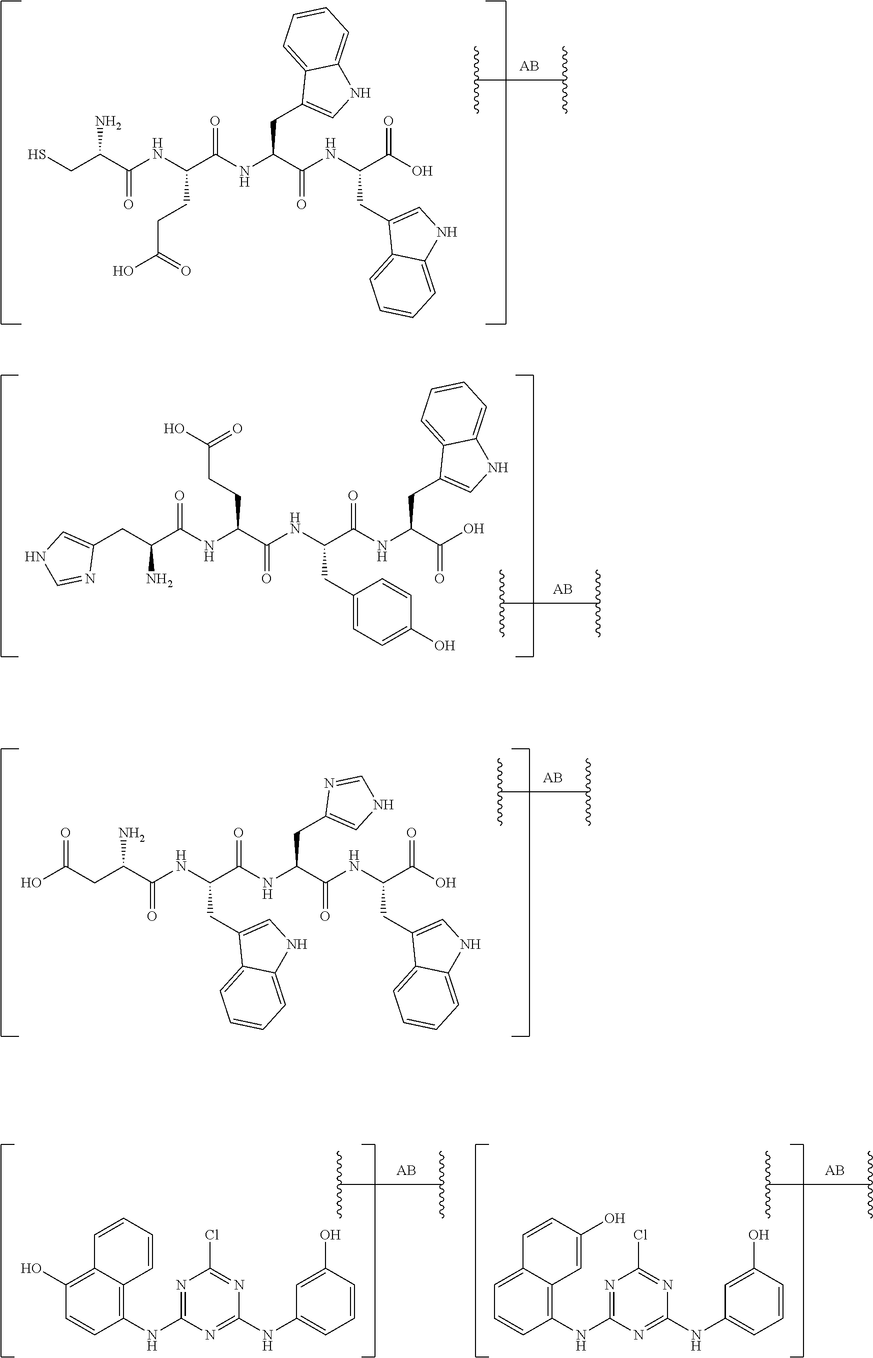

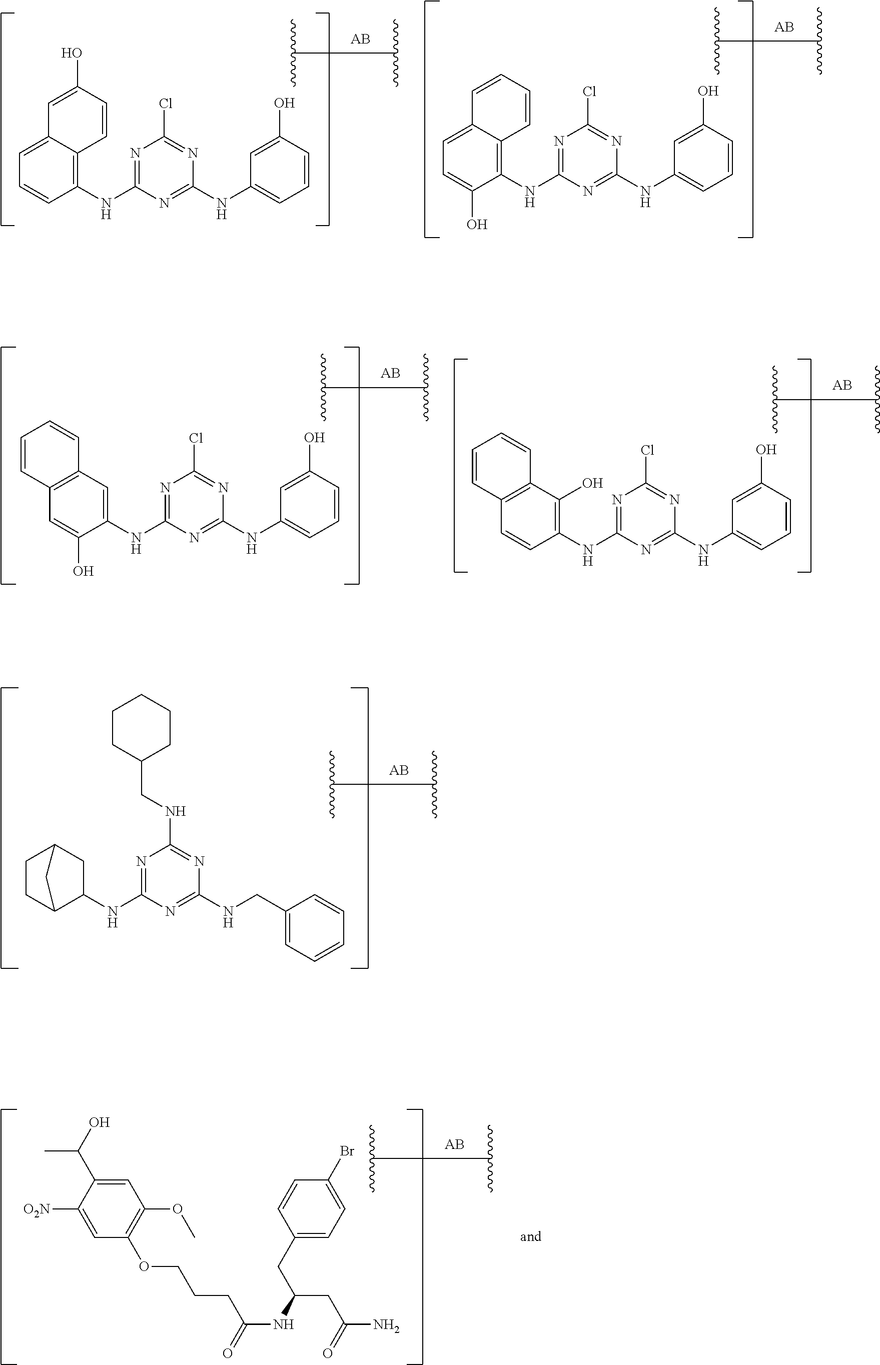

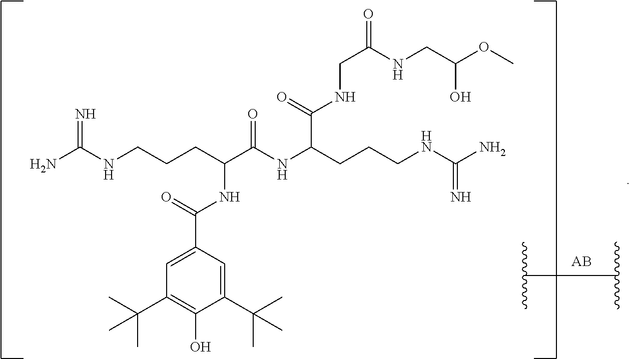

### Immunoglobulin E (IgE)

Immunoglobulin E (IgE) is a strong mediator of allergic disease, including but not limited to, atopic asthma, allergic rhinitis, atopic dermatitis, cutaneous contact hypersensitivity, IgE-mediated food allergy, IgE-mediated animal allergies, allergic conjunctivitis, allergic urticaria, anaphylactic shock, nasal polyposis, keratoconjunctivitis, mastocytosis, eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, monoclonal gammopathy of undetermined significance (MGUS), pemphigus vulgaris, mucus membrane pemphigoid, chronic urticaria, autoimmune uveitis, rheumatoid arthritis, autoimmune pancreatitis, and allergic rhinoconjunctivitis among others.

In certain embodiments the Immunoglobulin Targeting Ligand is:

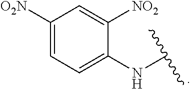 [SMILES: *Nc1ccc([N+](=O)[O-])cc1[N+](=O)[O-]]

### Anti-MAG IgM Autoantibodies

In some embodiments the Target Extracellular Protein is anti-MAG IgM autoantibodies. Myelin-associated glycoprotein (MAG) is a transmembrane glycoprotein that plays a role in glial-axonal interactions in the nervous system. In some patients, IgM anti-MAG antibodies develop leading to neuropathy. Antibody levels can be as high as four-fold over normal, leading to potential nephropathy. Lowering levels of anti-MAG antibodies is associated with clinical response in polyneuropathy.

Representative targeting ligands that bind to Anti-MAG IgM autoantibodies include HSO₃-3GlcAβ1-3Galβ1-4GlcNAcβ1-3Gal 1-4Glcβ1-Cer; HSO₃-3GlcAβ1-3Galβ1-4GlcNAcβ1-3Galβ1-4GlcNAcβ1-3Galβ1-4Glcβ1-Cer; HSO₃-3GlcAβ1-3Galβ1-4GlcNAc-X;

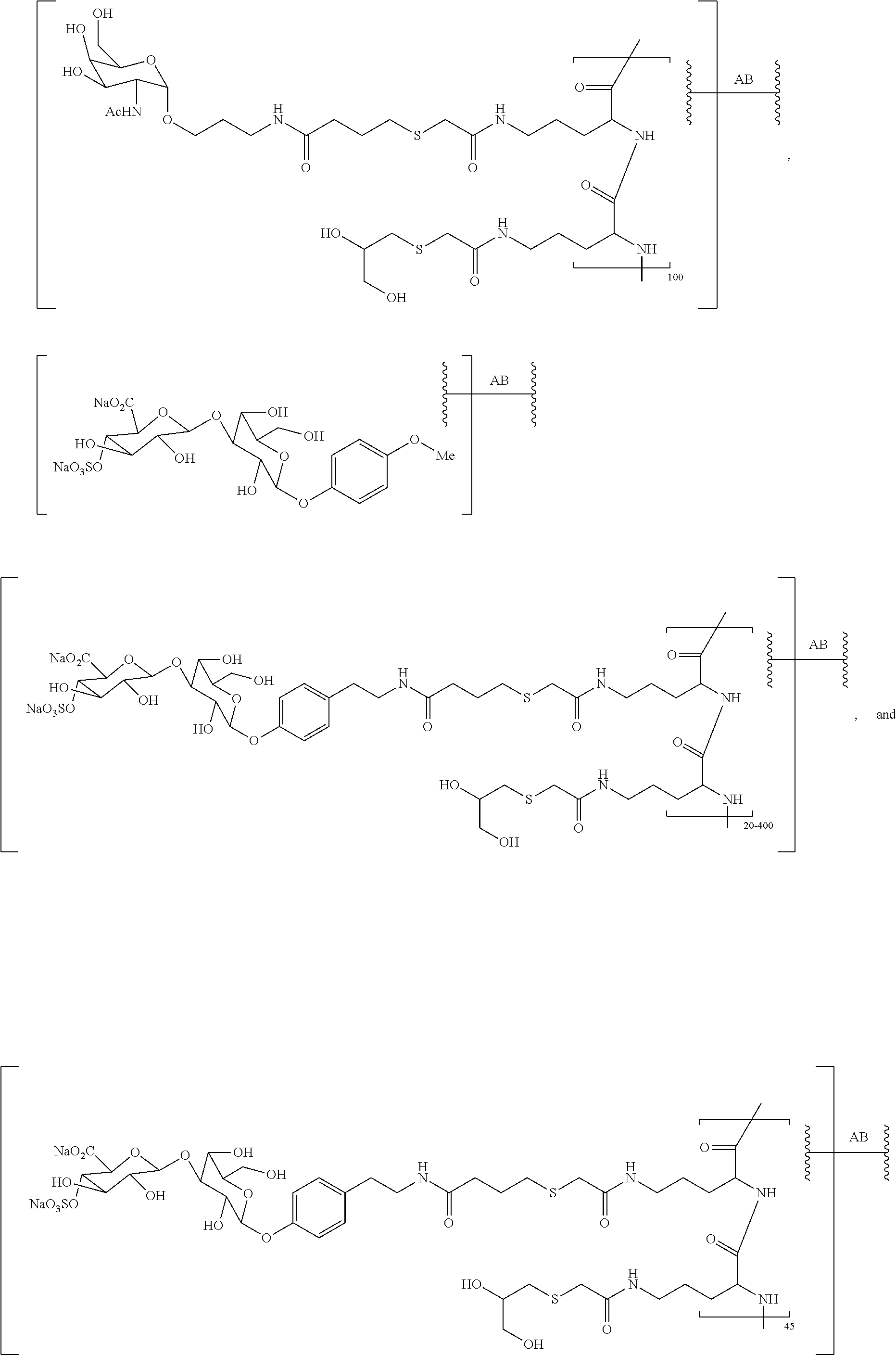

Additional IgM autoantibodies that can be used in the present invention are described in Herrendorff, R. et al. 2017 PNAS Early Edition, doi/10.1073/pnas.1619386114; and WO2018/167,230.

In certain embodiments the IgE Targeting Ligand is selected from

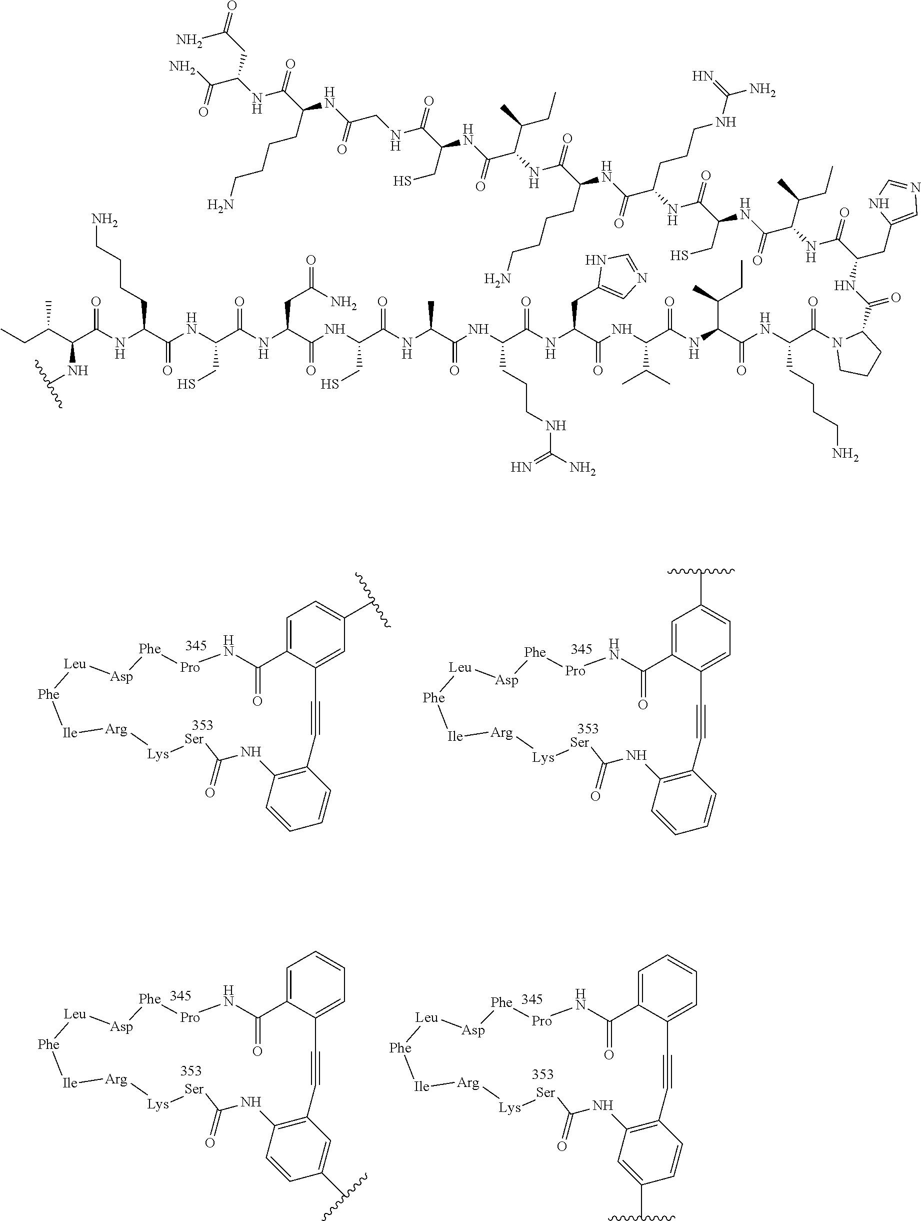

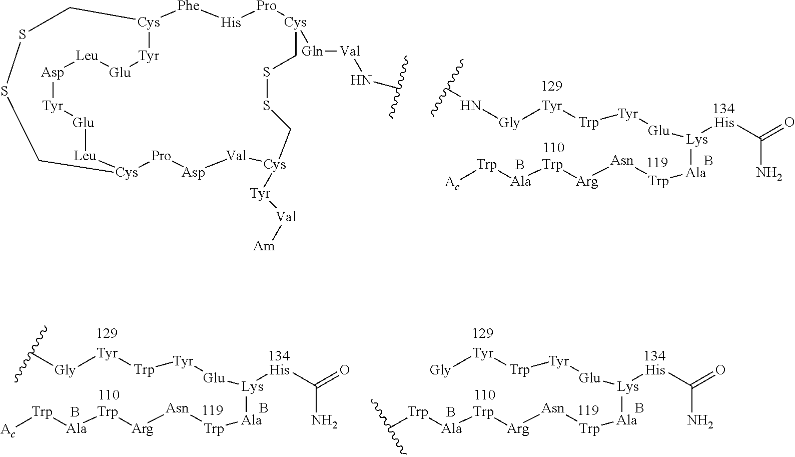

### Phospholipase A₂ Receptor-1 (PLA2R) Autoantibodies

In some embodiments, the Target Extracellular Protein is an autoantibody that binds PLA2R. Phospholipase A2 Receptor-1 (PLA2R) is a major target in autoimmune membranous nephropathy. Membranous nephropathy is one of the leading causes of nephrotic syndrome, with most patients progressing to end-stage renal disease. Current treatment regimes with anti-CD20 antibodies can be ineffective at generating a complete remission. PLA2R is a transmembrane glycoprotein with a cysteine-rich N-terminal extracellular domain. This domain contains the epitope where autoantibodies bind. Reduction of autoantibody levels may provide relief to patients and complete elimination of the autoantibodies could be required to produce a durable remission.

The Protein Data Bank provides the crystal structure of the CTLD7 domain of PLA2R, the region where autoantibodies bind (6JLI; Yu et al. J. Struct. Biol. 207, 295-300). Representative PLA2R autoantibody binding ligands include, but are not limited to,

SEQ ID NO: 126
GIFVIQSESLKKC
(Fresquet et al.
J. Am. Soc.
Nephrol 2015, 26, 302)
SEQ ID NO: 127
SVLTLENCK
(Fresquet et al.
J. Am. Soc.
Nephrol 2015, 26, 302)
SEQ ID NO: 128
SVLTLENC
(Brenchley et al.
WO2019/081912)
SEQ ID NO: 129
SVLTLDNCK
(Brenchley et al.
WO2019/081912)
SEQ ID NO: 130
SVLTEENC
(Brenchley et al.
WO2019/081912)
SEQ ID NO: 131
SVLTEENS
(Brenchley et al.
WO2019/081912)
SEQ ID NO: 132
SVLTDENC
(Brenchley et al.
WO2019/081912)
SEQ ID NO: 133
SVLTDENS
(Brenchley et al.
WO2019/081912)
SEQ ID NO: 134
PIQSESLKK
(Brenchley et al.
WO2019/081912)
SEQ ID NO: 135
VIDSESLKK
(Brenchley et al.
WO2019/081912)
SEQ ID NO: 136
PIDSESLKK
(Brenchley et al.
WO2019/081912)
SEQ ID NO: 137
VIQSESLKK
(Brenchley et al.
WO2019/081912)
SEQ ID NO: 138
PIESES-PEG-K-PEG-SVLTEENC
(Brenchley et al.
WO2019/081912)
SEQ ID NO: 139
VIQSES-PEG-K-PEG-SVL TLENC
(Brenchley et al.
WO2019/081912)
SEQ ID NO: 140
VIQSES-PEG-K-PEG-SVL TEENC
(Brenchley et al.
WO2019/081912)
SEQ ID NO: 141
PIDDES-PEG-K-PEG-SVLTLENC
(Brenchley et al.
WO2019/081912)
SEQ ID NO: 142
PIDDES-PEG-KPEG-SVLTEENC
(Brenchley et al.
WO2019/081912)
SEQ ID NO: 143
VIQSESLKKCKSVLTLENC
(Brenchley et al.
WO2019/081912)
SEQ ID NO: 144
PIQSESLKKCKSVLTLENC
(Brenchley et al.
WO2019/081912)
SEQ ID NO: 145
VIESESLKKCKSVLTLENC
(Brenchley et al.
WO2019/081912)
SEQ ID NO: 146
VIDSESLKKCKSVLTLENC
(Brenchley et al.
WO2019/081912)
SEQ ID NO: 147
PIESESLKKCKSVLTLENC
(Brenchley et al.
WO2019/081912)
SEQ ID NO: 148
VIQSESLKKCIQAGKLENC
(Brenchley et al.
WO2019/081912)
SEQ ID NO: 149
PIQSESLKKCIQAGKLENC
(Brenchley et al.
WO2019/081912)
SEQ ID NO: 150
VIESESLKKCIQAGKLENC
(Brenchley et al.
WO2019/081912)
SEQ ID NO: 151
VIDSESLKKCIQAGKLENC
(Brenchley et al.
WO2019/081912)
SEQ ID NO: 152
PIESESLKKCIQAGKLENC
(Brenchley et al.
WO2019/081912)
SEQ ID NO: 153
PIQSESLKKCKSVLTLENK
(Brenchley et al.
WO2019/081912)
SEQ ID NO: 154
VIESESLKKCKSVLTLENK
(Brenchley et al.
WO2019/081912)
SEQ ID NO: 155
VIDSESLKKCKSVLTLENK
(Brenchley et al.
WO2019/081912)
SEQ ID NO: 156
PIESESLKKCKSVLTLENK
(Brenchley et al.
WO2019/081912)
SEQ ID NO: 157
VIQSESLKKCIQAGKLENK
(Brenchley et al.
WO2019/081912)
SEQ ID NO: 158
PIQSESLKKCIQAGKLENK
(Brenchley et al.
WO2019/081912)
SEQ ID NO: 159
VIESESLKKCIQAGKLENK
(Brenchley et al.
WO2019/081912)
SEQ ID NO: 160
VIDSESLKKCIQAGKLENK
(Brenchley et al.
WO2019/081912)
SEQ ID NO: 161
PIESESLKKCIQAGKLENK
(Brenchley et al.
WO2019/081912)
SEQ ID NO: 162
PIESESGSVLTLENCK
(Brenchley et al.
WO2019/081912)
SEQ ID NO: 163
PIESESGGSVLTLENCK
(Brenchley et al.
WO2019/081912)
SEQ ID NO: 164
PIESESGGGSVLTLENCK
(Brenchley et al.
WO2019/081912)
SEQ ID NO: 165
PIESESGGGGSVLTLENCK
(Brenchley et al.
WO2019/081912)
SEQ ID NO: 166
PIESESGGGGGSVLTLENCK
(Brenchley et al.
WO2019/081912)
SEQ ID NO: 167
VIQSESGSVLTLENCK
(Brenchley et al.
WO2019/081912)
SEQ ID NO: 168
VIQSESGGSVLTLENCK
(Brenchley et al.
WO2019/081912)
SEQ ID NO: 169
VIQSESGGGSVLTLENCK
(Brenchley et al.
WO2019/081912)
SEQ ID NO: 170
VIQSESGGGGSVLTLENCK
(Brenchley et al.
WO2019/081912)
SEQ ID NO: 171
VIQSESGGGGGSVLTLENCK
(Brenchley et al.
WO2019/081912)
SEQ ID NO: 172
KGCFVIQSESLKKSIQAGKSVLTLENCK
(Brenchley et al.
WO2019/081912)
SEQ ID NO: 173
LKKCIQAGKSVLTLENCKQAN
(Brenchley et al.
WO2019/081912)
SEQ ID NO: 174
WQDKGIFVIQSESLKKCIQAGK
(Brenchley et al.
WO2019/081912)
SEQ ID NO: 175
KGIFVIQSESLKKCIQAGKSVLTLENCK
(Brenchley et al.
WO2019/081912)
SEQ ID NO: 176
GIFVIQSESLKKC
(Brenchley et al.
WO2015/185949)
SEQ ID NO: 177
WSVLTLENCK
(Brenchley et al.
WO2015/185949)
SEQ ID NO: 178
WQDKGIFVIQSESLKKCIQAGKSVLTLENCK
(Brenchley et al.
WO2015/185949)
SEQ ID NO: 179
YDWIPSSAW
(Glee et al., the journal of
immunology, 1999, 163:826-833)
SEQ ID NO: 180
AGAIWQRDW
SEQ ID NO: 181
AGAIWQKDW
SEQ ID NO: 182
VIQSESLK
SEQ ID NO: 183
PIQSESLK
SEQ ID NO: 184
PIESESLK
SEQ ID NO: 185
SVLTEENCK

In certain embodiments a compound is provided of Formula

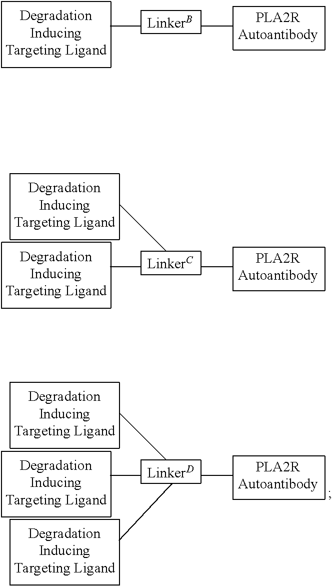

or a pharmaceutically acceptable salt thereof;
wherein

Degradation Inducing Targeting Ligand is selected from the group consisting of ASGPR Ligand^{A}, ASGPR Ligand^{B}, ASGPR Ligand^{C}, ASGPR Ligand^{D}, Mannose 6-Phosphate Ligand^{A} Mannose 6-Phosphate Ligand^{B}, and Mannose 6-Phosphate Ligand^{C}; and

PLA2R Autoantibody is any PLA2R autoantibody described in WO2019/081912.

In certain embodiments PLA2R Autoantibody is of Formula:

SEQ ID NO: 186:
SVLTXENX;
XIXXEX;
XENXK;
SEQ ID NO: 187:
SVLTXENCK;
SEQ ID NO: 188:
XIXXEXLK;
or

or a peptide of XIXXEX, XENXK, SEQ ID No: 186, 187, or 188 linked via a Linker-B group, in certain embodiments the linked sequences are SEQ ID: 186 and XIXXEX or SEQ ID NO: 186 and SEQ ID NO: 188;
wherein X is any natural amino acid or other amino acid described herein;
and wherein the sequence is linked to a Linker described herein at a terminal amine or carboxylic acid.

### Complement C3

In some embodiments the Target Extracellular Protein is complement C3. Complement C3 is one of the major proteins involved in the complement response, a significant factor in both innate and adaptive immunity. Elevated C3 is associated with Paroxysmal nocturnal hemoglobinuria (PNH), immune complex membranoproliferative glomerulonephritis (IC-MPGN), C3 glomerulopathy (C3G), geographic (GA), age-related macular degeneration (AMD), periodontitis, amyotrophic lateral sclerosis (ALS), hematopoietic stem cell transplantation-associated thrombotic microangiopathy (HSCT-TMA), cold agglutinin disease (CAD) and host attack in gene therapies. Reduction of C3 levels may ameliorate some of the symptoms or complications that arise from these inflammatory diseases.

The Protein Data Bank website provides the crystal structure of complement C3, searchable by 2A73 (Janssen, B. J. Nature, 2005, 505-511). Complement C3 bound to a nanobody inhibitor can be found with PDB accession code 6EHG (Jensen, R. K. et al. J Biol Chem, 2018, 293, 6269-6281). Nonlimiting examples of complement C3 binding ligands include

SEQ ID NO: 189
dYI*CV(N-Me)WQDWSarAHRC*(N-Me)I (Zhang, Y. et al. 2015, Immunobiology, 220, 993-998)
SEQ ID NO: 190
I*CVVQDWGHHRC*TAGMANLTSHASAI, (Sahu, A. et al. The Journal of Immunology, 1996, 157, 884-891).
SEQ ID NO: 191
I*CVVQDWGHHRC*T, (Sahu, A. et al. The Journal of Immunology, 1996, 157, 884-891).
SEQ ID NO: 192
*CVVQDWGHHAC* (Sahu, A. et al. The Journal of Immunology, 1996, 157, 884-891).
SEQ ID NO: 193
(Ac)-I*CVVQDWGHHRC*T-(NH₂), (Sahu, The Journal of Immunology, 2000, 165, 2491-2499);
SEQ ID NO: 194
*CVVQDWGHHRC*T-(NH₂), (Sahu, The Journal of Immunology, 2000, 165, 2491-2499);
SEQ ID NO: 195
*CVVQDWGHHRC *- (NH₂), (Sahu, The Journal of Immunology, 2000, 165, 2491-2499);
SEQ ID NO: 196
(Ac)-I*CVVGDWGHHRC*T-(NH2), (Sahu, The Journal of Immunology, 2000, 165, 2491-2499);
SEQ ID NO: 197
(Ac)-I*CVVQPWGHHRC*T-(NH2), (Sahu, The Journal of Immunology, 2000, 165, 2491-2499);
SEQ ID NO: 198
(Biotin)-KYSSI*CVVQDWGHHRC*T-(NH2), (Sahu, The Journal of Immunology, 2000, 165, 2491-2499);
SEQ ID NO: 199
(Ac)-dI*CVVQDWGHHRC*TAGHMANLTSHASAK-(Biotin), (Sahu, The Journal of Immunology, 2000, 165, 2491-2499);
SEQ ID NO: 200
(Ac)-dI*CV(N-Me)WQDWGAHRC*T, (Risitano et al. Blood, 2014, 123, 2094)
SEQ ID NO: 201
dYI*CV(N-Me)WQDW-Sar-AHRC*(N-Me)I, (Risitano et al. Blood, 2014, 123, 2094)

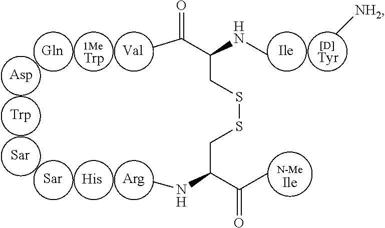

SEQ ID NO: 202
(PEG40K)-dYI*CV(N-Me)WQDW-Sar-AHRC*(N-Me)I (Risitano et al. Blood, 2014, 123, 2094)

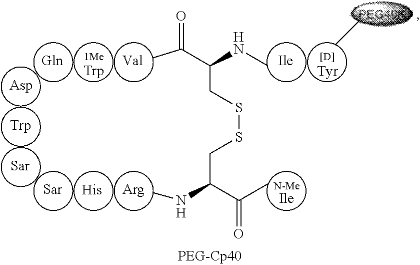

SEQ ID NO: 203: (Ac)-dYI*CV(N-Me)WQDW-Sar-AHRC*IK

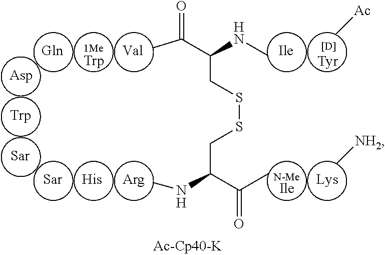

SEQ ID NO: 204: (Ac)-dYI*CV(N-Me)WQDW-Sar-AHRC*IK-(PEG40K)

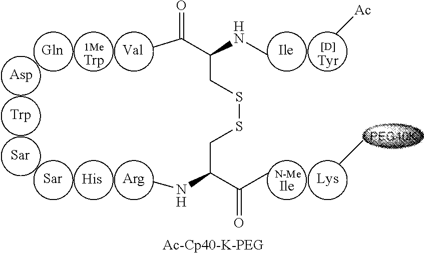

SEQ ID NO: 205
(Ac)-I*CVWQDWGAHRC*T-(NH₂) (Qu, H. et al. Immunobiology (2012) http://dx.doi.org/10.1016/j.imbio.2012.06.003)
SEQ ID NO: 206
(Ac)I*CV(N-Me)WQDW-Sar-AHRC*I-(NH₂) (Qu, H. et al. Immunobiology (2012) http://dx.doi.org/10.1016/j.imbio.2012.06.003)
SEQ ID NO: 207
(Ac)I*CV(N-Me)WQDW-Sar-AHRC*(N-Me)I-(NH₂) (Qu, H. et al. Immunobiology (2012) http://dx.doi.org/10.1016/j.imbio.2012.06.003)
SEQ ID NO: 208
(Ac)I*CV(N-Me)WQDWGAHRC*T-(NH₂) (Qu, H. et al. Molecular Immunology, 2011, 48, 481)
SEQ ID NO: 209
(Ac)X*CVXQDWGXXXC*T-(NH₂) (Mallik et al. J. Med. Chem., 2005, 48, 274-286)
SEQ ID NO: 210
(Ac)-I*CVVQDWGHHRC*T-(NH₂) (Mallik et al. J. Med. Chem., 2005, 48, 274-286)
SEQ ID NO: 211
(Ac)-I*CVVQDWGAHRC*T-(NH₂) (Mallik et al. J. Med. Chem., 2005, 48, 274-286)
SEQ ID NO: 212
(Ac)-I*CVTQDWGHHRC*T-(NH₂) (Mallik et al. J. Med. Chem., 2005, 48, 274-286)
SEQ ID NO: 213
(Ac)-I*CVSQDWGHHRC*T-(NH₂) (Mallik et al. J. Med. Chem., 2005, 48, 274-286)
SEQ ID NO: 214
(Ac)-I*CVHQDWGHHRC*T-(NH₂), (Mallik et al. J. Med. Chem., 2005, 48, 274-286)
SEQ ID NO: 215
(Ac)-I*CVFQDWGHHRC*T-(NH₂), (Mallik et al. J. Med. Chem., 2005, 48, 274-286)
SEQ ID NO: 216
(Ac)-I*CVYQDWGAHRC*T-(NH₂), (Mallik et al. J. Med. Chem., 2005, 48, 274-286)
SEQ ID NO: 217
(Ac)-I*CVWQDWGWHRC*T-(NH₂), (Mallik et al. J. Med. Chem., 2005, 48, 274-286)
SEQ ID NO: 218
(Ac)-I*CVWQDWGHHRC*T-(NH₂), (Mallik et al. J. Med. Chem., 2005, 48, 274-286)
SEQ ID NO: 219
(Ac)-I*CVWQDWGAHRC*T, (Mallik et al. J. Med. Chem., 2005, 48, 274-286)
SEQ ID NO: 220
(Ac)-I*CVWQDWGAHRC*T-(NH₂), (Mallik et al. J. Med. Chem., 2005, 48, 274-286)
SEQ ID NO: 221
(Ac)-I*CVWQDWGAdHRC*T, (Mallik et al. J. Med. Chem., 2005, 48, 274-286)
SEQ ID NO: 222
(Ac)-I*CVWQDWGdAHRC*T, (Mallik et al. J. Med. Chem., 2005, 48, 274-286)
SEQ ID NO: 223
(Ac)-dI*CVWQDWGAHRC*T, (Mallik et al. J. Med. Chem., 2005, 48, 274-286)
SEQ ID NO: 224
(Ac)-I*CVWQDWGAHRC*dT, (Mallik et al. J. Med. Chem., 2005, 48, 274-286)
SEQ ID NO: 225
(Ac)-I*CVWQDWGAHRC*T-(NH₂), (Lopez de Victoria, A. et al. Chem Biol Drug Des 2011, 77, 431-440)
SEQ ID NO: 226
W*CVWQDWGTNRC*W-(NH₂), (Lopez de Victoria, A. et al. Chem Biol Drug Des 2011, 77, 431-440)
SEQ ID NO: 227
(Ac)-D*CVWQDWGTNKC*W-(NH₂), (Lopez de Victoria, A. et al. Chem Biol Drug Des 2011, 77, 431-440)
SEQ ID NO: 228
Q*CVWQDWGQNQC*W-(NH₂), (Lopez de Victoria, A. et al. Chem Biol Drug Des 2011, 77, 431-440)
SEQ ID NO: 229
(Ac)-I*CVWQDWGAHRC*W-(NH₂), (Lopez de Victoria, A. et al. Chem Biol Drug Des 2011, 77, 431-440)
SEQ ID NO: 230
(Ac)-W*CVWQDWGAHRC*T-(NH₂), (Lopez de Victoria, A. et al. Chem Biol Drug Des 2011, 77, 431-440)
SEQ ID NO: 231
(Ac)-W*CVWQDWGAHRC*W-(NH₂), (Lopez de Victoria, A. et al. Chem Biol Drug Des 2011, 77, 431-440)
SEQ ID NO: 232
(Ac)-I*AVWQDWGAHR-Hcy*T-(NH₂), (Knerr, P. et al. ACS Chem. Biol., 2011, 6, 753-760)
SEQ ID NO: 233
(Ac)-I*CVWQDWGAHRC*(N-Me)I-(NH₂), (Knerr, P. et al. ACS Chem. Biol., 2011, 6, 753-760)
SEQ ID NO: 234
(Ac)-I*AVWQDWGAHR-Hcy*(N-Me)I-(NH₂), (Knerr, P. et al. ACS Chem. Biol., 2011, 6, 753-760)
SEQ ID NO: 235
(Ac)-I*CV(5f)WQDWGAHRC*T-(NH₂), (Katragadda et al. J. Med. Chem. 2006, 49, 4616-4622).
SEQ ID NO: 236
(Ac)-I*CV(5-Me)WQDWGAHRC*T-(NH₂), (Katragadda et al. J. Med. Chem. 2006, 49, 4616-4622).
SEQ ID NO: 237
(Ac)-I*CV(2-Nal)QDWGAHRC*T-(NH₂), (Katragadda et al. J. Med. Chem. 2006, 49, 4616-4622).
SEQ ID NO: 238
(Ac)-I*CVWQD(5-f)WGAHRCT-(NH₂), (Katragadda et al. J. Med. Chem. 2006, 49, 4616-4622).
SEQ ID NO: 239
(Ac)-I*CVWQD(5-Me)WGAHRCT-(NH₂), (Katragadda et al. J. Med. Chem. 2006, 49, 4616-4622).
SEQ ID NO: 240
(Ac)-I*CVWQD(1-Me)WGAHRCT-(NH₂), (Katragadda et al. J. Med. Chem. 2006, 49, 4616-4622).
SEQ ID NO: 241
(Ac)-I*CVYQDWGAHRC*T-(CONH2), (WO 2021/007,111)
SEQ ID NO: 242
(Ac)-I*CVWQDWGAHRC*T-(COOH), (WO 2021/007,111)
SEQ ID NO: 243
(Ac)-I*CVWQDWGAHRC*T-(CONH2), (WO 2021/007,111)
SEQ ID NO: 244
(Ac)-I*CVWQDWGAHRC*dT-(COOH), (WO 2021/007,111)
SEQ ID NO: 245
(Ac)-I*CV(2-Nal)QDWGAHRC*T-(CONH2), (WO 2021/007,111)
SEQ ID NO: 246
(Ac)-I*CV(2-Nal)QDWGAHRC*T-(COOH), (WO 2021/007,111)
SEQ ID NO: 247
(Ac)-I*CV(1-Nal)QDWGAHRC*T-(COOH), (WO 2021/007,111)
SEQ ID NO: 248
(Ac)-I*CV(2-lal)QDWGAHRC*T-(CONH2), (WO 2021/007,111)
SEQ ID NO: 249
(Ac)-I*CV(2-lal)QDWGAHRC*T-(COOH), (WO 2021/007,111)
SEQ ID NO: 250
(Ac)-I*CVDhtQDWGAHRC*T-(COOH), (WO 2021/007,111)
SEQ ID NO: 251
(Ac)-I*CV(Boa)QDWGAHRC*T-(COOH), (WO 2021/007,111)
SEQ ID NO: 252
(Ac)-I*CV(Bpa)QDWGAHRC*T-(CONH2), (WO 2021/007,111)
SEQ ID NO: 253
(Ac)-I*CV(Bta)QDWGAHRC*T-(COOH), (WO 2021/007,111)
SEQ ID NO: 254
(Ac)-I*CV(Bta)QDWGAHRC*T-(CONH2), (WO 2021/007,111)
SEQ ID NO: 255
(Ac)-I*CVWQDWG(2-Abu)HRC*T-(CONH2), (WO 2021/007,111)
SEQ ID NO: 256
H-GI*CVWQDWGAHRC*TAN-(COOH), (WO 2021/007,111)
SEQ ID NO: 257
(Ac)-I*CV(5f)WQDWGAHRC*T-(CONH2), (WO 2021/007,111)
SEQ ID NO: 258
(Ac)-I*CV(5-me)WQDWGAHRC*T-(CONH2), (WO 2021/007,111)
SEQ ID NO: 259
(Ac)-I*CV(1-me)WIQDWGAHRC*T-(CONH2), (WO 2021/007,111)
SEQ ID NO: 260
(Ac)-I*CVWQD(5f)WGAHRC*T-(CONH2), (WO 2021/007,111)
SEQ ID NO: 261
(Ac)-I*CV(5-f)WQD(5f)WGAHRC*T-(CONH2), (WO 2021/007,111)
SEQ ID NO: 262
(Ac)-I*CV(5-me)WQD(5f)WGAHRC*T-(CONH2), (WO 2021/007,111)
SEQ ID NO: 263
(Ac)-I*CV(1-me)WQD(5f)WGAHRC*T-(CONH2), (WO 2021/007,111)
SEQ ID NO: 264
H-GI*CV(6f)WQD(6f)WGAHRC*TN-(COOH), (WO 2021/007,111)
SEQ ID NO: 265
(Ac)-I*CV(1-formvl)WQDWGAHRC*T-(CONH2), (WO 2021/007,111)
SEQ ID NO: 266
(Ac)-I*CV(1-methyoxy)WQDWGAHRC*T-(CONH2), (WO 2021/007,111)
SEQ ID NO: 267
H-GI*CV(5-f)WQD(5-f)WGAHRC*TN-(COOH), (WO 2021/007,111)
In certain embodiments the complement C3 targeting ligand is

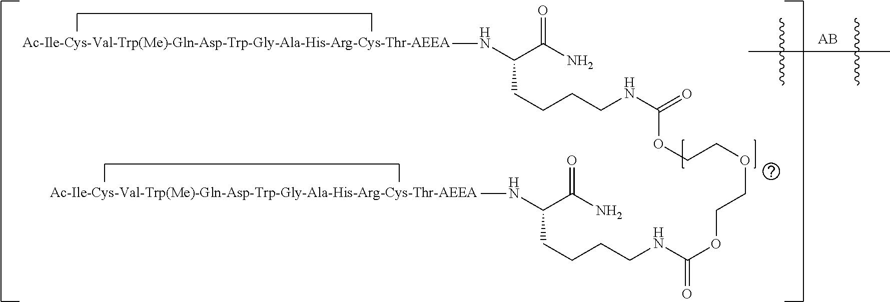

SEQ ID NO: 268
(Ac)I*CV(Me)WQDWGAHRC*T-(AEEA)- . . .

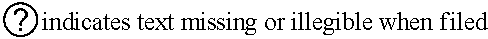

### Complement C1q

In some embodiments, the Target Extracellular Protein is Complement C1q. The complement system is part of the innate immune system and clears apoptotic cells and pathogens. Activation of this pathway begins with binding the C1 complex to an immunoglobulin that has bound to an antigen. The C1 complex consists of C1q and a tetramer of proteases (C1r and C1s). C1 q mediates the binding of complement to IgG or IgM. Following the binding event, the proteases are activated, and they cleave C4 which sets off the remainder of the pathway that ends in opsonization. Overactivity of this pathway can lead to a number of inflammatory pathologies including allograft rejection, neuromyelitis optica, generalized myasthenia gravis, and cold agglutinin disease. Degradation of C1q may reduce the symptoms associated with these inflammatory diseases.

The Protein Data Bank website provides the crystal structure of Complement C1q searchable by 2JG9 (Paidassi, H. et al., J. Immunol, 2008, 180, 2329-2338), 1PK6 (Gaboriaud, C., J. Biol. Chem, 2003, (278) 46974-46982), 5HZF (Moreau, C. et al., Front. Immunol, 2016, (7) 79), 2WNV and 2WNU (Garlatti, V. et. al., J. Immunol. 2010, (185), 808). Also provided on the PDB website is the structure of complement C1q with a ligand bound, searchable by 6Z67 (Laursen, N. et al. Front. Immunol., 2020, (11), 1504)

Nonlimiting examples of complement C1q binding ligands include

SEQ ID NO: 269
(Ac)-AEAKAKA-(CONH2)
(WO 88/07054)
SEQ ID NO: 270
IALILEPICCQERAA
(U.S. Pat. No. 8,906,845; Sharp, J. A. et al.
PLoS ONE 10(7), e0132446)
SEQ ID NO: 271
IALILEPICCQERAA-(dPEG24)
(Sharp, J. A. et al. PLoS ONE 10(7), e0132446)
SEQ ID NO: 272
(dPEG24)-IALILEPICCQERAA
(Sharp, J. A. et al. PLoS ONE 10(7), e0132446)
SEQ ID NO: 273
RALILEPICCQERAA
(Sharp, J. A. et al. PLoS ONE 10(7), e0132446)
SEQ ID NO: 274
IRLILEPICCQERAA
(Sharp, J. A. et al. PLoS ONE 10(7), e0132446)
SEQ ID NO: 275
IARILEPICCQERAA
(Sharp, J. A. et al. PLoS ONE 10(7), e0132446)
SEQ ID NO: 276
IALIREPICCQERAA
(Sharp, J. A. et al. PLoS ONE 10(7), e0132446)
SEQ ID NO: 277
IALILEPICCRERAA
(Sharp, J. A. et al. PLoS ONE 10(7), e0132446)
SEQ ID NO: 278
IALILEPICCORRAA
(Sharp, J. A. et al. PLoS ONE 10(7), e0132446)
SEQ ID NO: 279
IELILEPICCQERAA
(Sharp, J. A. et al. PLoS ONE 10(7), e0132446)
SEQ ID NO: 280
IAEILEPICCQERAA
(Sharp, J. A. et al. PLoS ONE 10(7), e0132446)
SEQ ID NO: 281
IALILEPICCQEEAA
(Sharp, J. A. et al. PLoS ONE 10(7), e0132446)
SEQ ID NO: 282
IALILEPICCQEREA
(Sharp, J. A. et al. PLoS ONE 10(7), e0132446)
SEQ ID NO: 283
IALILEEICCQERAA
(Sharp, J. A. et al. PLoS ONE 10(7), e0132446)
SEQ ID NO: 284
IALILEPECCQERAA
(Sharp, J. A. et al. PLoS ONE 10(7), e0132446)
SEQ ID NO: 285
PAICQRATATLGTVGSNTSGTTAIEACILL
(U.S. Pat. No. 8,906,845; Sharp, J.
A. et al. Frontiers in Immunology
(2014) 5, 406)
SEQ ID NO: 286
*CEGPFGPRHDLTFC*W
(Roos, A. et al. The Journal of Immunology,
2001, 167, 7052)
SEQ ID NO: 287
*XbEGPFGPRHDLTFC*W
(Roos, A. et al. The Journal of Immunology,
2001, 167, 7052)
SEQ ID NO: 288
QYYPFSX
(Messmer B.T. et al. Molecular Immunology,
2000, 37, 343)
SEQ ID NO: 289
NPFNLAR
(Messmer B.T. et al. Molecular Immunology,
2000, 37, 343)
SEQ ID NO: 290
QLQDMTSSPFWL
(Messmer B.T. et al. Molecular Immunology,
2000, 37, 343)
SEQ ID NO: 291
NPFVIGRWHPPH
(Messmer B.T. et al. Molecular Immunology,
2000, 37, 343)
SEQ ID NO: 292
SLAKFLNPFLYR
(Messmer B.T. et al. Molecular Immunology,
2000, 37, 343)
SEQ ID NO: 293
ASTPRFEPFQLD
(Messmer B.T. et al. Molecular Immunology,
2000, 37, 343)
SEQ ID NO: 294
SLHSQPYSPFML
(Messmer B.T. et al. Molecular Immunology,
2000, 37, 343)
SEQ ID NO: 295
NILSSWSSPFVF
(Messmer B.T. et al. Molecular Immunology,
2000, 37, 343)
SEQ ID NO: 296
NLPSSWTNPFYL
(Messmer B.T. et al. Molecular Immunology,
2000, 37, 343)
SEQ ID NO: 297
SPFMLHP
(Messmer B.T. et al. Molecular Immunology,
2000, 37, 343)
SEQ ID NO: 298
PSPFMLT
(Messmer B.T. et al. Molecular Immunology,
2000, 37, 343)
SEQ ID NO: 299
IGPFHLH
(Messmer B.T. et al. Molecular Immunology,
2000, 37, 343)
SEQ ID NO: 300
TNPFMLN
(Messmer B.T. et al. Molecular Immunology,
2000, 37, 343)
SEQ ID NO: 301
NTTFLYP
(Messmer B.T. et al. Molecular Immunology,
2000, 37, 343)
SEQ ID NO: 302
SHYTQYL
(Messmer B.T. et al. Molecular Immunology,
2000, 37, 343)
SEQ ID NO: 303
NHHPNYW
(Messmer B.T. et al. Molecular Immunology,
2000, 37, 343)
SEQ ID NO: 304
VHYPLSW
(Messmer B.T. et al. Molecular Immunology,
2000, 37, 343)
SEQ ID NO: 305
HHLKYSDTSPPI
(Messmer B.T. et al. Molecular Immunology,
2000, 37,343)
SEQ ID NO: 306
SHMHERWDTSPPI
(Messmer B.T. et al. Molecular Immunology,
2000, 37, 343)
SEQ ID NO: 307
SHMHERWDTSYQ
(Messmer B.T. et al. Molecular Immunology,
2000, 37, 343)
SEQ ID NO: 308
SHIHSNAAWRIT
(Messmer B.T. et al. Molecular Immunology,
2000, 37,343)
SEQ ID NO: 309
WHYPHWQ
(Messmer B.T. et al. Molecular Immunology,
2000, 37, 343)
SEQ ID NO: 310
SHYLYTQ
(Messmer B.T. et al. Molecular Immunology,
2000, 37, 343)
SEQ ID NO: 311
AHYSFTQ
(Messmer B.T. et al. Molecular Immunology,
2000, 37, 343)
SEQ ID NO: 312
THYPTFY
(Messmer B.T. et al. Molecular Immunology,
2000, 37, 343)
SEQ ID NO: 313
EHNTSFW
(Messmer B.T. et al. Molecular Immunology,
2000, 37, 343)
SEQ ID NO: 314
NHYKLTW
(Messmer B.T. et al. Molecular Immunology,
2000, 37, 343)
SEQ ID NO: 315
NHSPYFQ
(Messmer B.T. et al. Molecular Immunology,
2000, 37, 343)
SEQ ID NO: 316
SHYQHYQ
(Messmer B.T. et al. Molecular Immunology,
2000, 37, 343)
SEQ ID NO: 317
PAICQRATATLGTVGSNTSGTTEIEACILL
(U.S. Pat. No. 8,906,845;
Gronemus, J.Q. et al.
Molecular immunology, 2010, 48, 305)
SEQ ID NO: 318
WLGLGGGYGW
(Lauvrak V., Biol. Chem. 1997, 378, 1509)
SEQ ID NO: 319
FYGPFFLNDSLRGIW
(Lauvrak V., Biol. Chem. 1997, 378, 1509)
SEQ ID NO: 320
LRFLNPFSLDGSGFW
(Lauvrak V., Biol. Chem. 1997, 378, 1509)
SEQ ID NO: 321
HSPFCLGVLECFGLV
(Lauvrak V., Biol. Chem. 1997, 378, 1509)
SEQ ID NO: 322
TCGAFYLYHDPFICG
(Lauvrak V., Biol. Chem. 1997, 378, 1509)
SEQ ID NO: 323
MQHCLASHELYLPWC
(Lauvrak V., Biol. Chem. 1997, 378, 1509)
SEQ ID NO: 324
FFVFGSGDAFAFSDM
(Lauvrak V., Biol. Chem. 1997, 378, 1509)
SEQ ID NO: 325
PCVIIDTGSSRWCYL
(Lauvrak V., Biol. Chem. 1997, 378, 1509)
SEQ ID NO: 326
HSPFCLGVLECFGLV
(Lauvrak V., Biol. Chem. 1997, 378, 1509)
SEQ ID NO: 327
HAAFEPRGDVRHTLL
(Lauvrak V., Biol. Chem. 1997, 378, 1509)
SEQ ID NO: 328
CRWDGSWGEVRC
(Lauvrak V., Biol. Chem. 1997, 378, 1509)
SEQ ID NO: 329
CYWVGTWGEAVC
(Lauvrak V., Biol. Chem. 1997, 378, 1509)
SEQ ID NO: 330
RWFPCPNKEGCCSISV
(Lauvrak V., Biol. Chem. 1997, 378, 1509)
SEQ ID NO: 331
RSTYCNKNKDSCHIPE
(Lauvrak V., Biol. Chem. 1997, 378, 1509)
SEQ ID NO: 332
QPPQCIKDGGFVICRV
(Lauvrak V., Biol. Chem. 1997, 378, 1509)
SEQ ID NO: 333
KGKKCKPEEHPCNEPM
(Lauvrak V., Biol. Chem. 1997, 378, 1509)
SEQ ID NO: 334
NKMTCSDDGKLCWEHL
(Lauvrak V., Biol. Chem. 1997, 378, 1509)
SEQ ID NO: 335
PLGRPCPTCPLAPS
(Lauvrak V., Biol. Chem. 1997, 378, 1509)
SEQ ID NO: 336
QRMRPCPSCPLAPW
(Lauvrak V., Biol. Chem. 1997, 378, 1509)
SEQ ID NO: 337
WPSRPCPSCPEVPP
(Lauvrak V., Biol. Chem. 1997, 378, 1509)
SEQ ID NO: 338
SCTKDCPTCPLVPV
(Lauvrak V., Biol. Chem. 1997, 378, 1509)
C1qNb75 Nanobody
(Laursen, N. S. et al. Frontiers in
Immunology, 2020, 11, 1504)
SEQ ID NO: 339
PAIAQRATATLGTVGSNTSGTTEIEACILL
(U.S. Pat. No. 8,906,845)
SEQ ID NO: 340
PAICQRATATLGTVGSNTSGTTEIEAAILL
(U.S. Pat. No. 8,906,845)
SEQ ID NO: 341
PAICQRATATLGTVGSNTSGTTAIBACILL
(U.S. Pat. No. 8,906,845)
SEQ ID NO: 342
PAICQRATATLGTVGSNTSGTTEIAACILL
(U.S. Pat. No. 8,906,845)
SEQ ID NO: 343
PAICQRABIEACILL
(U.S. Pat. No. 8,906,845)
SEQ ID NO: 344
PAIAQRAEIEAAILL
(U.S. Pat. No. 8,906,845)
SEQ ID NO: 345
PAICQRATATLGTNTSGTTEIBACILL
(U.S. Pat. No. 8,906,845)
SEQ ID NO: 346
PAICQRATATLSGTTEIEACILL
(U.S. Pat. No. 8,906,845)
SEQ ID NO: 347
PAICQRATATTEIBACILL
(U.S. Pat. No. 8,906,845)
SEQ ID NO: 348
AICQRATATLGTVGSNTSGTTEIEACILL
(U.S. Pat. No. 8,906,845)
SEQ ID NO: 349
ICQRATATLGTVGSNTSGTTEIEACILL
(U.S. Pat. No. 8,906,845)
SEQ ID NO: 350
CQRATATLGTVGSNTSGTTEIEACILL
(U.S. Pat. No. 8,906,845)
SEQ ID NO: 351
PAICQRATATLGTVGSNTSGTTEIEACIL
(U.S. Pat. No. 8,906,845)
SEQ ID NO: 352
PAICQRATATLGTVGSNTSGTTEIEACI
(U.S. Pat. No. 8,906,845)
SEQ ID NO: 353
PAICQRATATLGTVGSNTSGTTEIEAC
(U.S. Pat. No. 8,906,845)
SEQ ID NO: 354
(Ac)-IALILEPICCQERAA
(U.S. Pat. No. 8,906,845)
SEQ ID NO: 355
(Ac)-PAICQRATATLGTVGSNTSGTTEIEACILL
(U.S. Pat. No. 8,906,845)
SEQ ID NO: 356
(Ac)-PAIAQRATATLGTVGSNTSGTTEIEACILL
(U.S. Pat. No. 8,906,845)
SEQ ID NO: 357
(Ac)-PAICQRATATLGTVGSNTSGTTEIEAAILL
(U.S. Pat. No. 8,906,845)
SEQ ID NO: 358
(Ac)-PAICQRATATLGTVGSNTSGTTAIEACILL
(U.S. Pat. No. 8,906,845)
SEQ ID NO: 359
(Ac)-PAICQRATATLGTVGSNTSGTTEIAACILL
(U.S. Pat. No. 8,906,845)
SEQ ID NO: 360
(Ac)-PAICQRAEIEACILL
(U.S. Pat. No. 8,906,845)
SEQ ID NO: 361
(Ac)-PAIAQRABIEAAILL
(U.S. Pat. No. 8,906,845)
SEQ ID NO: 362
(Ac)-PAICQRATATLGTNTSGTTEIEACILL
(U.S. Pat. No. 8,906,845)
SEQ ID NO: 363
(Ac)-PAICQRATATLSGTTEIEACILL
(U.S. Pat. No. 8,906,845)
SEQ ID NO: 364
(Ac)-PAICQRATATTEIEACILL
(U.S. Pat. No. 8,906,845)
SEQ ID NO: 365
(Ac)-ICQRATATLGTVGSNTSGTTEIEACILL
(U.S. Pat. No. 8,906,845)
SEQ ID NO: 366
(Ac)-CQRATATLGTVGSNTSGTTEIEACILL
(U.S. Pat. No. 8,906,845)
SEQ ID NO: 367
(Ac)-PAICQRATATLGTVGSNTSGTTEIEACIL
(U.S. Pat. No. 8,906,845)
SEQ ID NO: 368
(Ac)-PAICQRATATLGTVGSNTSGTTEIBACI
(U.S. Pat. No. 8,906,845)
SEQ ID NO: 369
(Ac)-PAICQRATATLGTVGSNTSGTTEIEAC
(U.S. Pat. No. 8,906,845)

In certain embodiments the Linker is bound through the C-terminus of the amino acid sequence for example

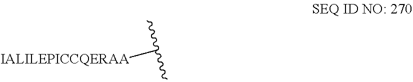

In certain embodiments the Linker is bound to the N-terminus for example

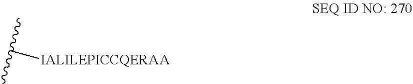

In certain embodiments the complement C3 Targeting Ligand is selected from:

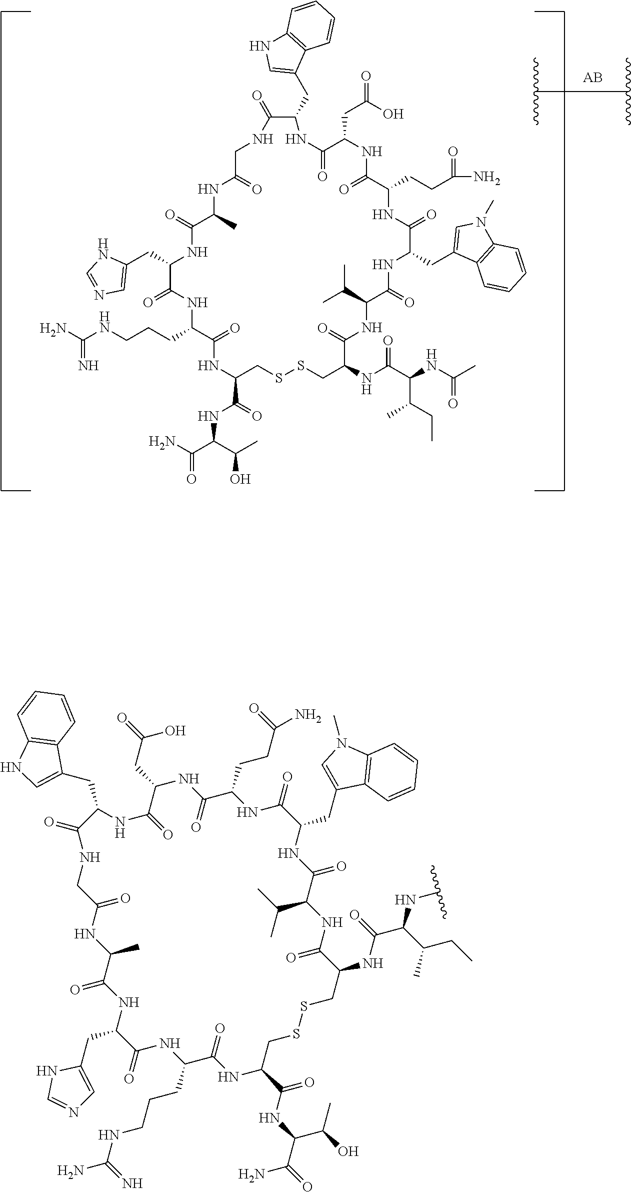

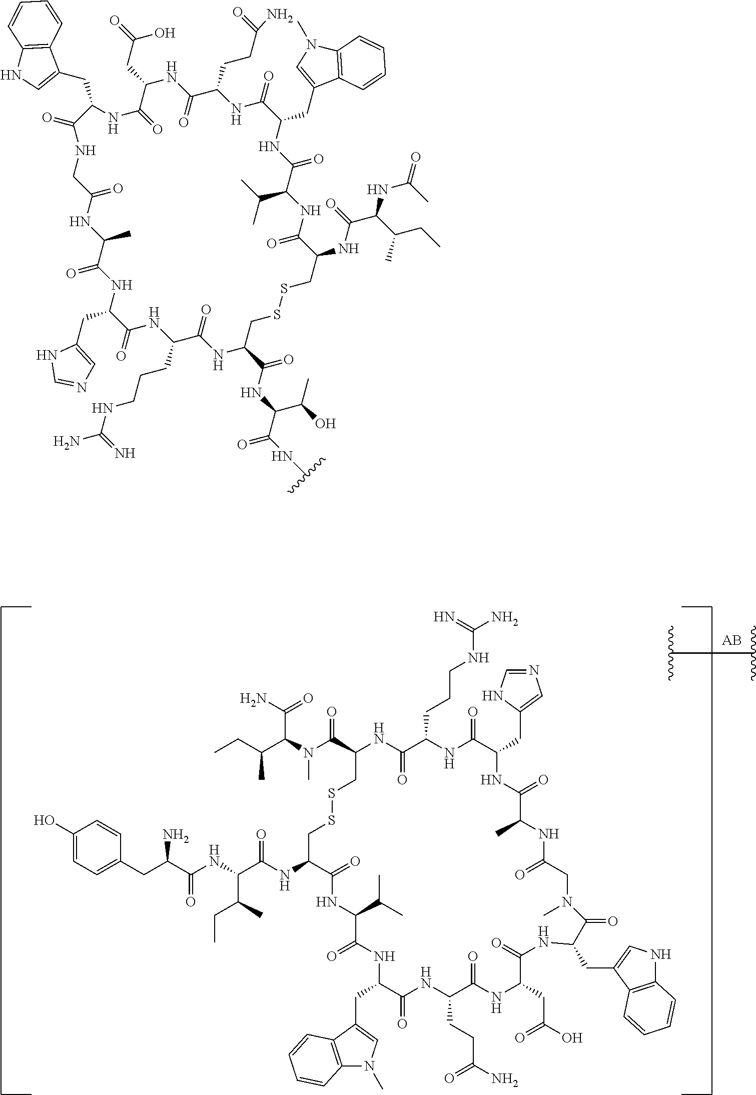

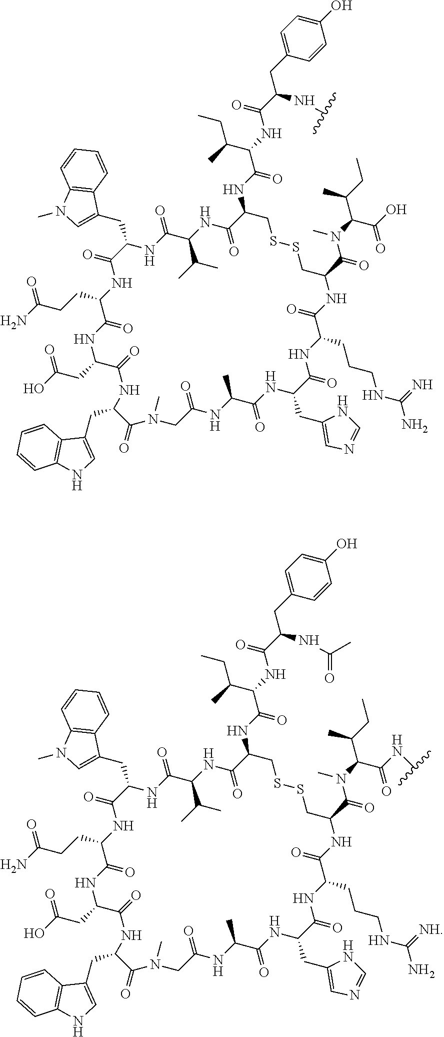

### IL-17

In some embodiments, the Target Extracellular Protein is human interleukin-17 (IL-17) (UniProtKB-Q16552 (IL17_HUMAN)). Interleukin-17 is a 35 kDa homodimeric glycoprotein and is an important cytokine for the inflammatory response. IL-17 is secreted by a distinct class of Helper T cells (known as Th17 cells) which mediates tissue inflammation. A characteristic effect of IL-17 production is the expansion of neutrophils, and in healthy tissue it is responsible for neutrophil homeostasis. IL-17 has been implicated as a major factor in psoriasis as well as other autoimmune diseases. Other diseases where IL-17 therapies may be of benefit include but are not limited to asthma, rheumatoid arthritis, psoriatic arthritis, Crohn's disease, and inflammatory bowel disease. Inflammation caused by IL-17 has been shown to hamper recovery post-stroke.

The Protein Data Bank website provides the crystal structure of IL-17, searchable by 4NUX (Zhang, B. et al. (2014) Acta Crystallogr D Biol Crystallogr 70: 1476-1483), 4HSA (Liu, S. et al. (2013) Nat Commun 4: 1888-1888), 4QHU (unpublished), 6WIR (Lieu, R. et al. (2020) PLoS One 15: e0232311-e0232311), 5VB9 (Ting, J. P. et al. (2018) PLoS One 13: e0190850-e0190850), 4NUX (Zhang, et al. (2014) Acta Crystallogr D Biol Crystallogr 70: 1476-1483), 3JVF (Ely, L. K. et al. (2009) Nat Immunol 10: 1245-1251), 5N9B (unpublished), 2VXS (Gerhardt, S. et al. (2009) J Mol Biol 394: 905).

Non-limiting examples of IL-17 Targeting Ligands can be found in, for example, WO2012101263A1, WO2020163554A1, WO2021055376A1, WO2020146194A1, WO2020127685A1, US20150005319, WO2014066726A2, WO2019223718A1, WO2020135872A1, WO2020146194A1, WO2021027721A1, WO2021027724, WO2021027729A1, WO2021067191A1, CN104069102A, CN105601617B, CN108299256B, Liu et al. “Binding site elucidation and structure guided design of macrocyclic IL-17A antagonists” 2016, Scientific Reports, 6:30859., Liu et al. “Inhibiting complex IL-17AA and IL-17RA interactions with a linear peptide” 2016, Scientific Reports 6:26071. Wang, W. et al. “Artificial macrocycles as IL-17A/IL-17RA antagonists”. Med. Chem. Comm. 2018, 9, 22. Liu, C. et al. “The flavonoid cyanidin blocks binding of the cytokine interleukin-17A to the IL-17RA subunit to alleviate inflammation in vivo” Science Signaling 10, eaaf8823 (2017).

Additional binding ligands include

SEQ ID NO: 370
TVVTAPADLWDWIRA
(Liu et al. 2016, Scientific Reports 6:
26071)
SEQ ID NO: 371
ITVTMPADLWDWIRA
(Liu et al. 2016, Scientific Reports 6:
26071)
SEQ ID NO: 372
IVVTIPADLWDWIRA
(Liu et al. 2016, Scientific Reports 6:
26071)
SEQ ID NO: 373
IVVTLPADLWDWIRA
(Liu et al. 2016, Scientific Reports 6:
26071)
SEQ ID NO: 374
IVVTVPADLWDWIRA
(Liu et al. 2016, Scientific Reports 6:
26071)
SEQ ID NO: 375
IVVTMPADLWDWIMA
(Liu et al. 2016, Scientific Reports 6:
26071)
SEQ ID NO: 376
IVVTMPADLWDWINA
(Liu et al. 2016, Scientific Reports 6:
26071)
SEQ ID NO: 377
IVVTMPADLWDWIQA
(Liu et al. 2016, Scientific Reports 6:
26071)
SEQ ID NO: 378
IHVTIPADLWDWINK
(Liu et al. 2016, Scientific Reports 6:
26071)
SEQ ID NO: 379
IHVTIPADLWDWIN
(Liu et al. 2016, Scientific Reports 6:
26071)

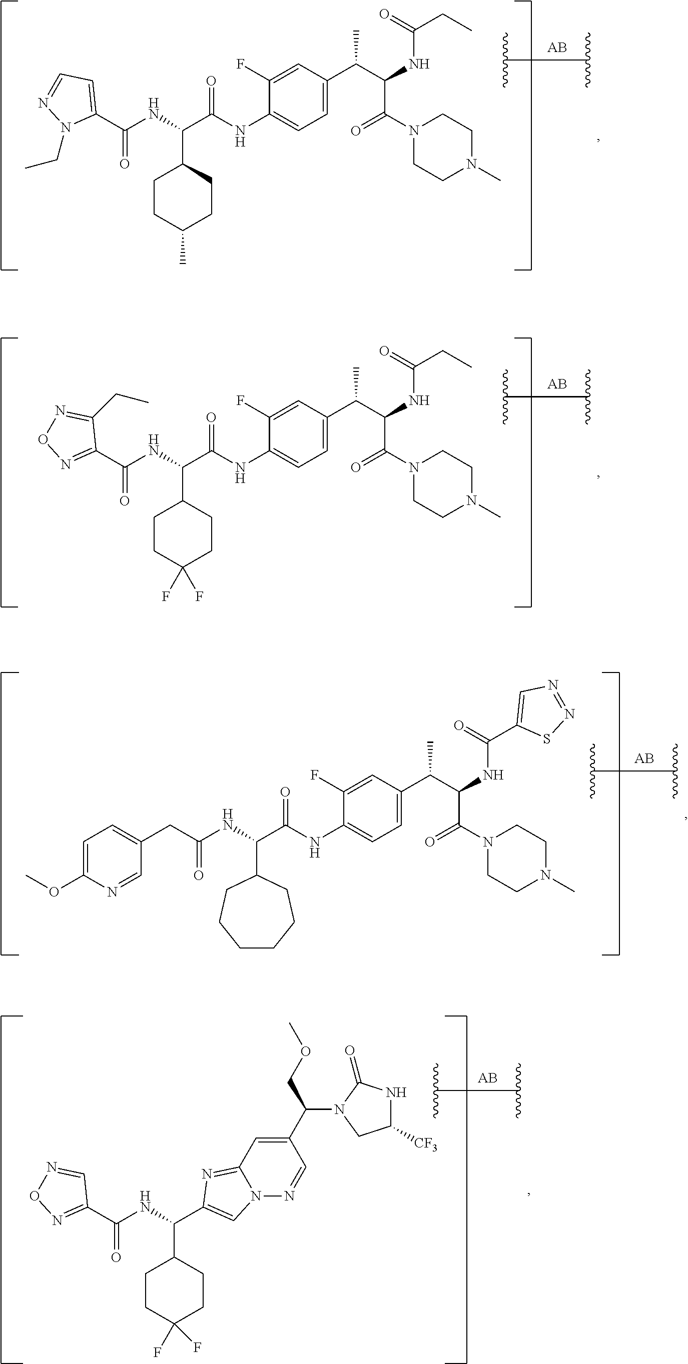

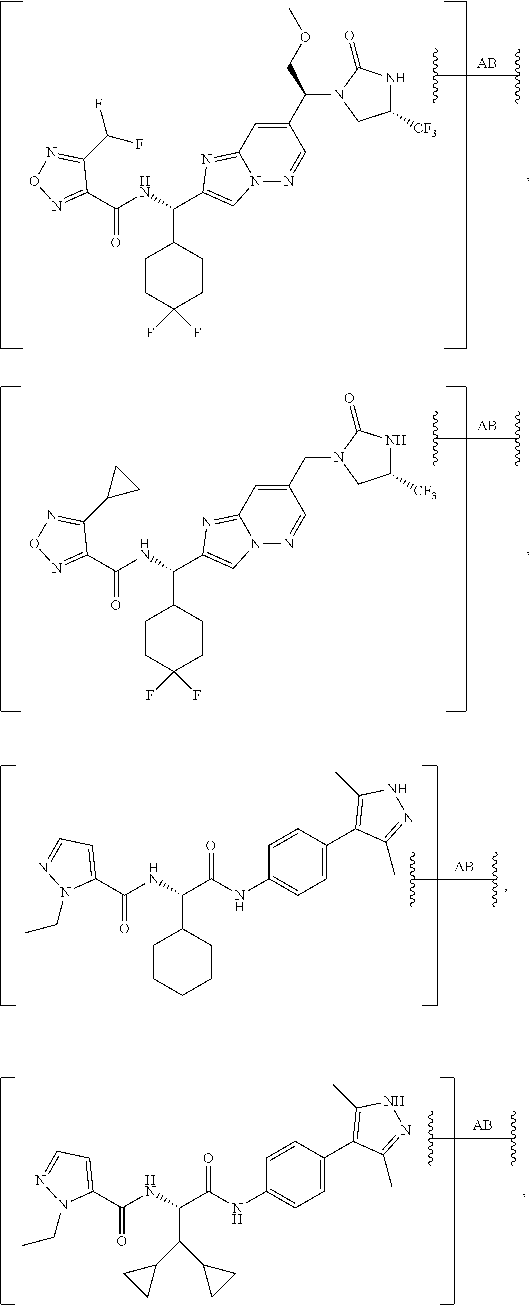

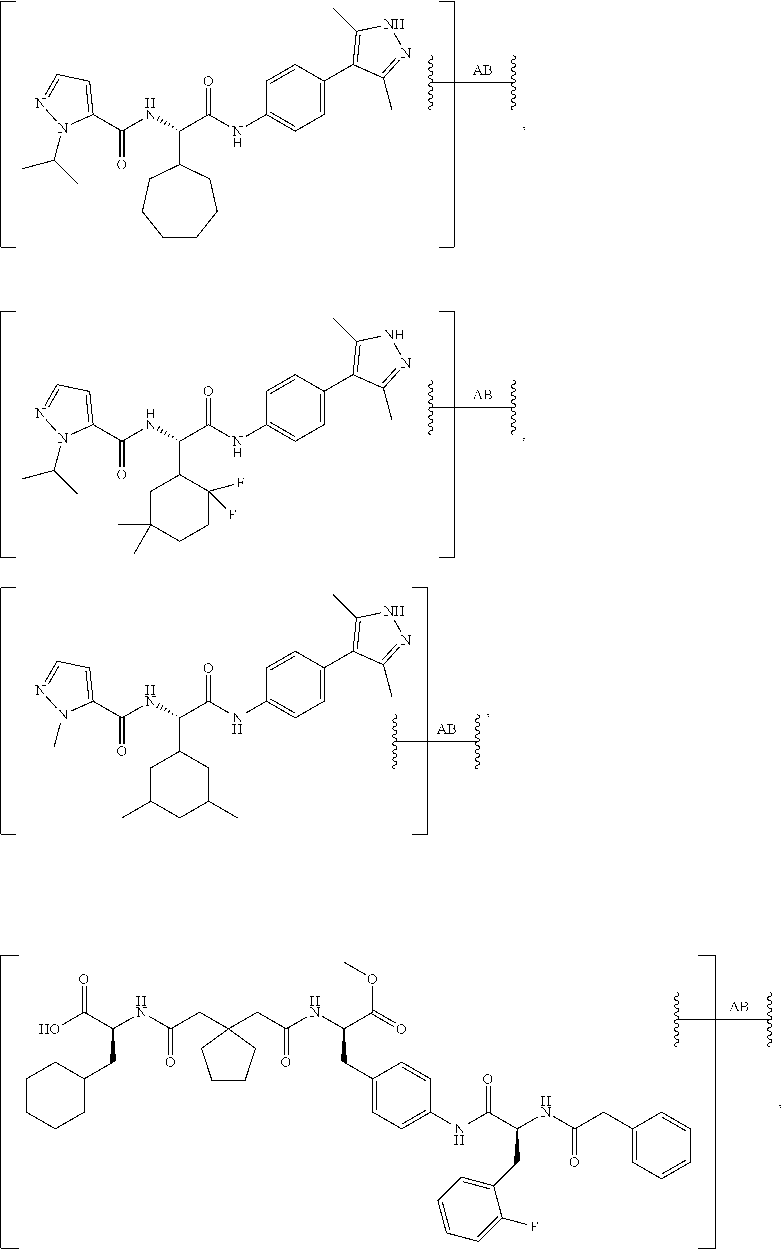

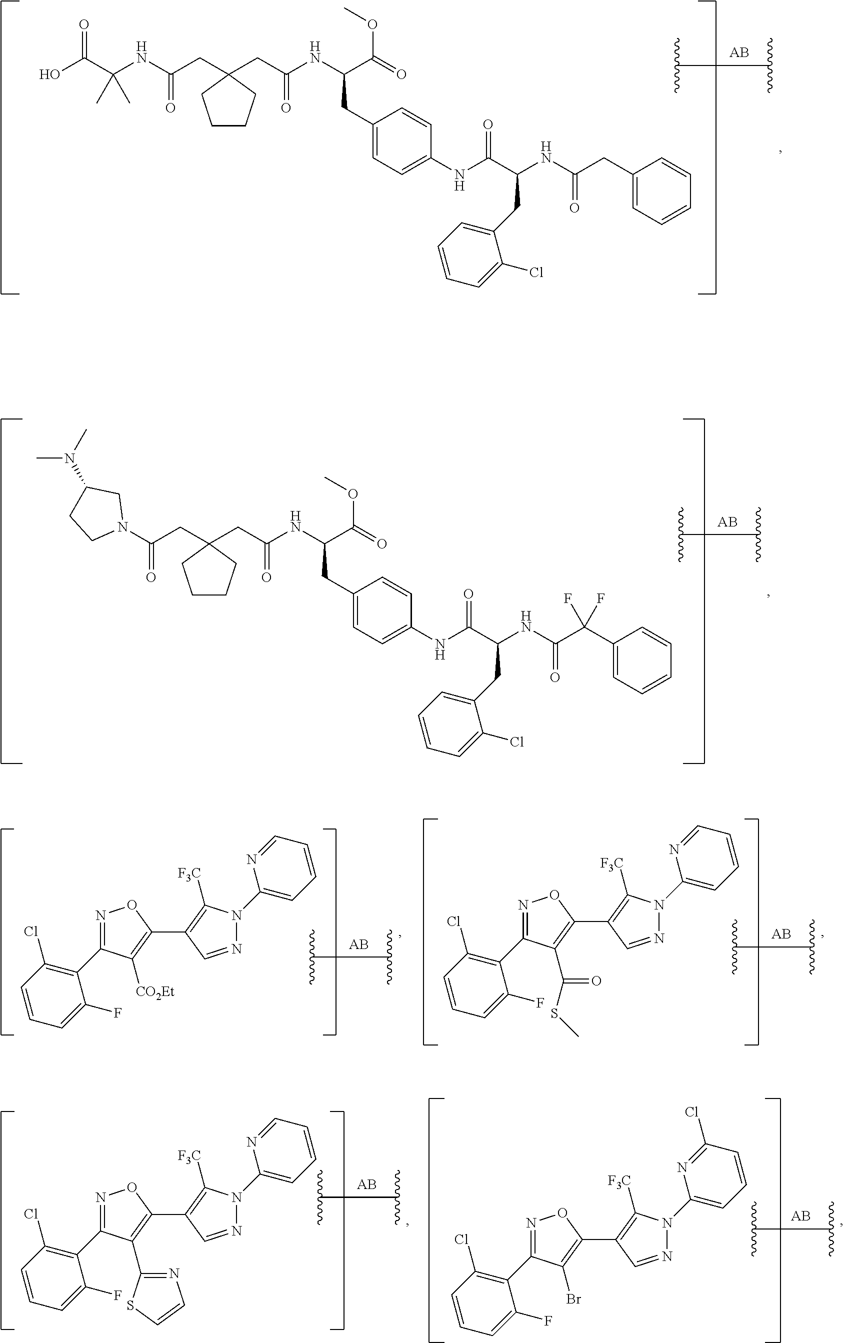

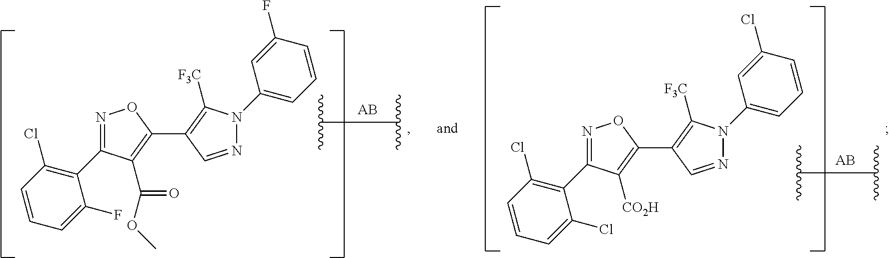

each of which is optionally substituted with 1, 2, 3, or 4 substituents independently selected from R²¹.

In certain embodiments a compound is provided of Formula

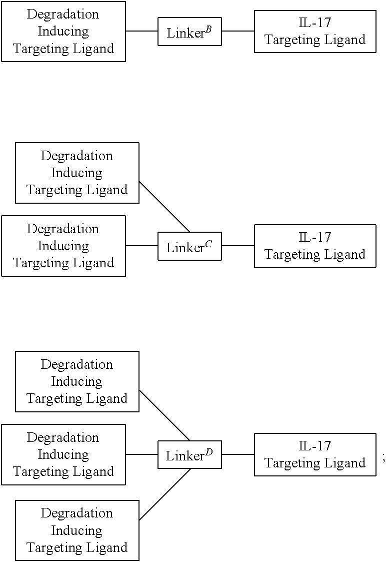

or a pharmaceutically acceptable salt thereof,
wherein

Degradation Inducing Targeting Ligand is selected from the group consisting of ASGPR 5 Ligand^{A}, ASGPR Ligand^{B}, ASGPR Ligand^{C}, ASGPR Ligand^{D}, Mannose 6-Phosphate Ligand^{A}Mannose 6-Phosphate Ligand^{B}, and Mannose 6-Phosphate Ligand^{C}; and

IL-17 Targeting Ligand is any IL-17 ligand described in WO2020/146,194; WO2020/163,554; WO2020/127,685; and WO2021/055,376; each of which is incorporated by reference.

In certain embodiments Degradation Inducing Targeting Ligand is selected from ASGPR Ligand^{A}.

In certain embodiments Degradation Inducing Targeting Ligand is selected from ASGPR Ligand^{B}.

In certain embodiments Degradation Inducing Targeting Ligand is selected from ASGPR Ligand^{C}.

In certain embodiments Degradation Inducing Targeting Ligand is selected from ASGPR Ligand^{D}.

In certain embodiments Degradation Inducing Targeting Ligand is selected from Mannose 6-Phosphate Ligand^{A}.

In certain embodiments Degradation Inducing Targeting Ligand is selected from Mannose 6-Phosphate Ligand^{B}.

In certain embodiments Degradation Inducing Targeting Ligand is selected from Mannose 6-Phosphate Ligand^{C}.

In certain embodiments IL-17 Targeting Ligand is of Formula:

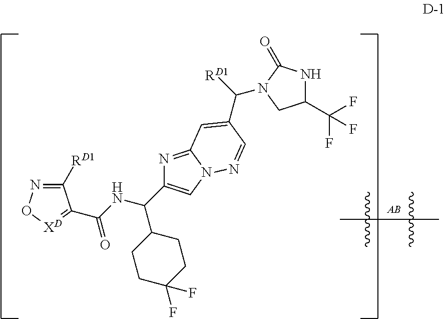

wherein,
X^{D} is CH or N;
R^{D1} is —CH₃, —CH₂F, —CHF₂, —CF₃, —CH₂CH₃, —CH₂CF₃, —CH(CH₃)₂, CH₂CHF₂, CH₂CH₂F, —CF(CH₃)₂, CF₂CH₃, —OCH₃,

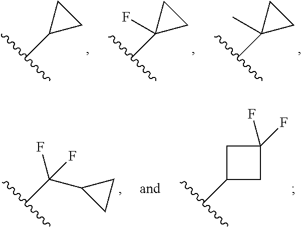

R^{D2} is —H or —CH₂OCH₃.

In certain embodiments IL-17 Targeting Ligand is of Formula:

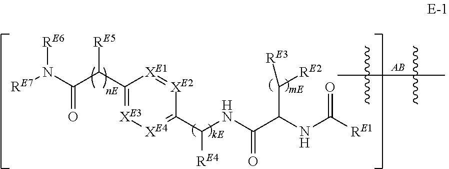

wherein,
R^{E1} is alkyl, substituted alkyl, aryl, substituted aryl, heteroaryl, substituted heteroaryl, arylalkyl, substituted arylalkyl, heteroarylalkyl, substituted heteroarylalkyl —OR^{E8} or —NR^{E9}R^{E10} or an F pocket substituent;
R^{E2} is alkyl, substituted alkyl, heterocycle, substituted heterocycle, aryl, substituted aryl, fused cycloalkylaryl, substituted fused cycloalkylaryl, heteroaryl, substituted heteroaryl or a D pocket substituent;
each R^{E3} is independently hydrogen, (C₁-C₇) alkyl, (C₁-C₇) substituted alkyl or —OR^{E32};
m^{E} is 0, 1 or 2;
each R^{E4} is independently hydrogen, (C₁-C₇) alkyl, (C₁-C₇) substituted alkyl, cycloalkyl substituted cycloalkyl, heterocycle or substituted heterocycle;
k^{E} is 0 or 1;
X^{E1}, X^{E2}, X^{E3} and X^{E4} are independently —N— or —CR^{E11}— provided that no more than two of X^{E1}, X^{E2}, X^{E3} and X^{E4} are nitrogen;
each R^{E5} is independently hydrogen, (C₁-C₇) alkyl, (C₁-C₇) substituted alkyl, heterocycle, substituted heterocycle, cycloalkyl, substituted cycloalkyl, heterocyclealkyl, substituted heterocyclealkyl, —NR^{E12}R^{E13}, —NR^{E14}C(O)R^{E15}, —NHSO₂R^{E31}, OH or a B pocket substituent;
R^{E6} is hydrogen or alkyl;
R^{E7} is heterocycle, substituted heterocycle, —(CHR^{E16})ₒR^{E17} or —(CHR^{E18})ₚR^{E19} or R^{E6} and R^{E7} taken together with the nitrogen atom to which they are attached form piperazine, substituted piperazine, heterocycle or substituted heterocycle,

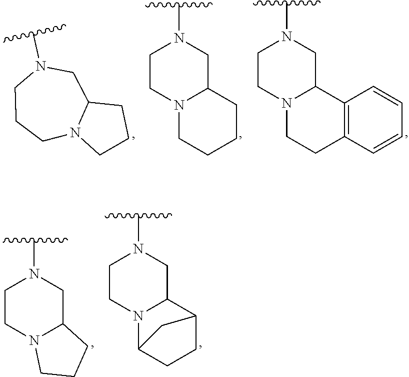

or an A pocket substituent;
R^{E8} is (C₁-C₇) alkyl, (C₁-C₇) substituted alkyl, aryl, substituted aryl, heteroaryl or substituted heteroaryl;
each R^{E11} is independently hydrogen, alkyl, substituted alkyl, —OR^{E20}, —NR^{E21}R^{E22}, halo, —CN, —CO₂R^{E23}, —CONR^{E24}R^{E25} or —SR^{E26};
n^{E} is 1, 2 or 3;
o^{E} is 1, 2 or 3;
p^{E} is 1, 2 or 3;
each R^{E16} is independently hydrogen, (C₁-C₇) alkyl or (C₁-C₇) substituted ALKYL
R^{E17} is

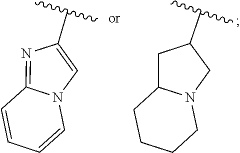 [SMILES: *c1cn2ccccc2n1 ; *C1CC2CCCCN2C1]

each R^{E18} is independently hydrogen, (C₁-C₇) alkyl, or (C₁-C₇) substituted alkyl; R^{E19} is —NR^{E27}R^{E28}.
R^{E27} and R^{E28} together with the nitrogen atom to which they are attached form a heterocycle or substituted heterocycle ring or

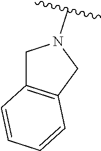 [SMILES: *N1Cc2ccccc2C1]

R^{E9}, R^{E10}, RE¹², RE¹³, RE¹⁴, R^{E15}, RE¹⁸, RE¹⁹, R^{E20}, R^{E21}, R^{E22}, R^{E23}, R^{E24}, R^{E25}, R^{E26}, R^{E30}, R^{E31}, and R^{E32} are independently selected at each instance from hydrogen, alkyl, substituted alkyl, heterocycle, substituted heterocycle, aryl, substituted aryl, heteroaryl, substituted heteroaryl, or alternatively, independently, R^{E9} and R^{E10}, R^{E21} and R^{E22} and R^{E24} and R^{E25} together with atom to which they are attached form a cycloalkyl, substituted cycloalkyl, cycloheteroalkyl or substituted cycloheteroalkyl ring;
R^{E28} is hydrogen or alkyl;
A-pocket substituent is selected from the group consisting of

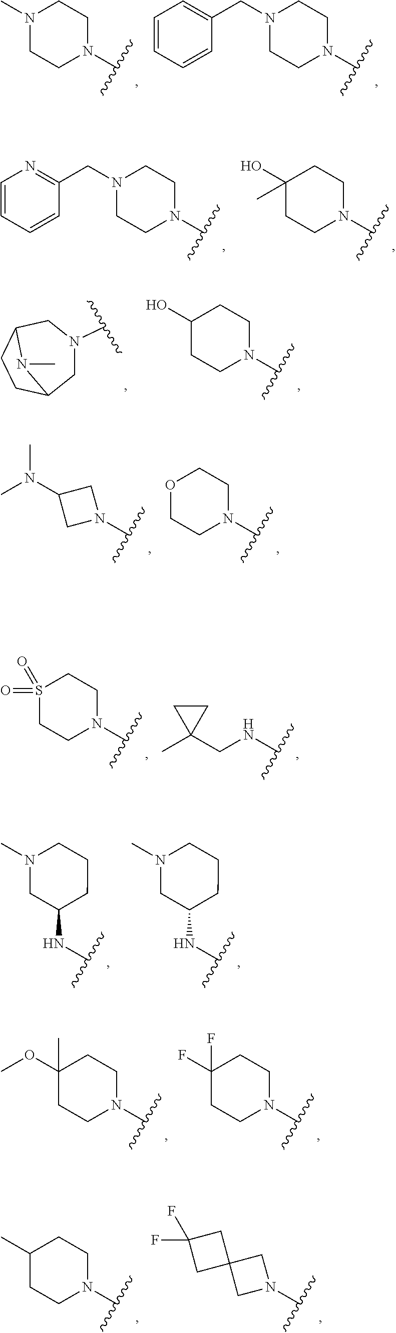

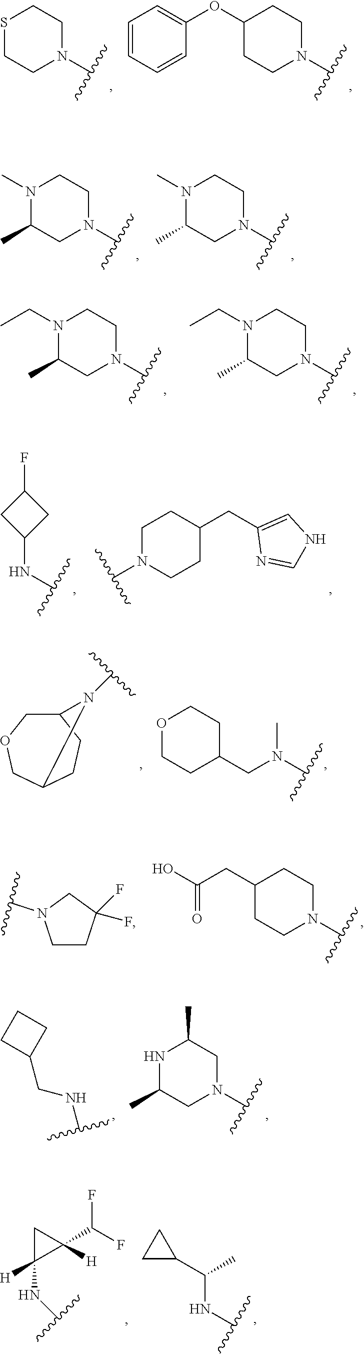

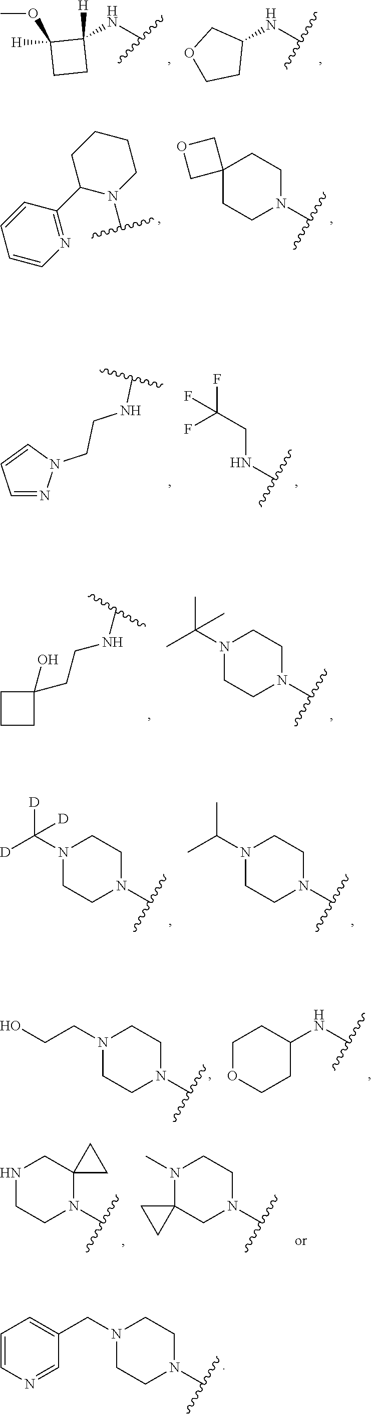

B-pocket substituent is selected from the group consisting of

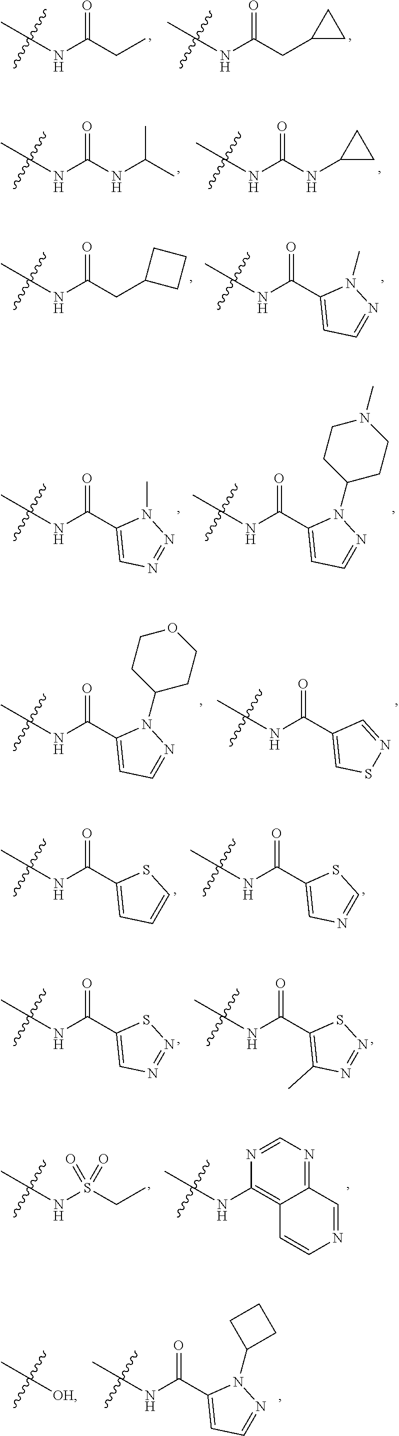

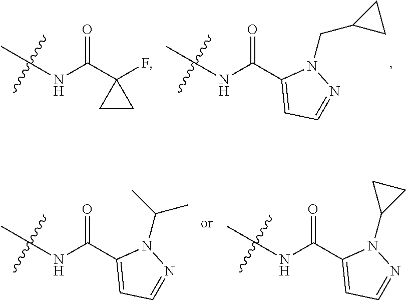 [SMILES: *NC(=O)C1(F)CC1 ; *NC(=O)c1ccnn1CC1CC1 ; *NC(=O)c1ccnn1C(C)C ; *NC(=O)c1ccnn1C1CC1]

D-pocket substituent is selected from the group consisting of

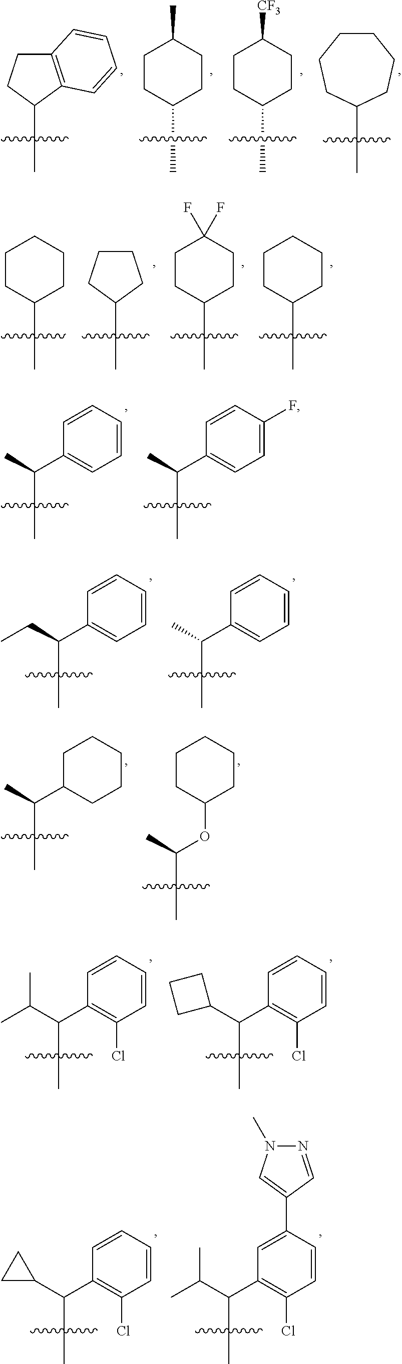

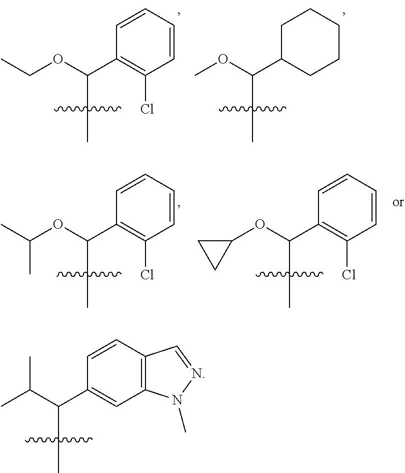

F-pocket substituent is selected from the group consisting of

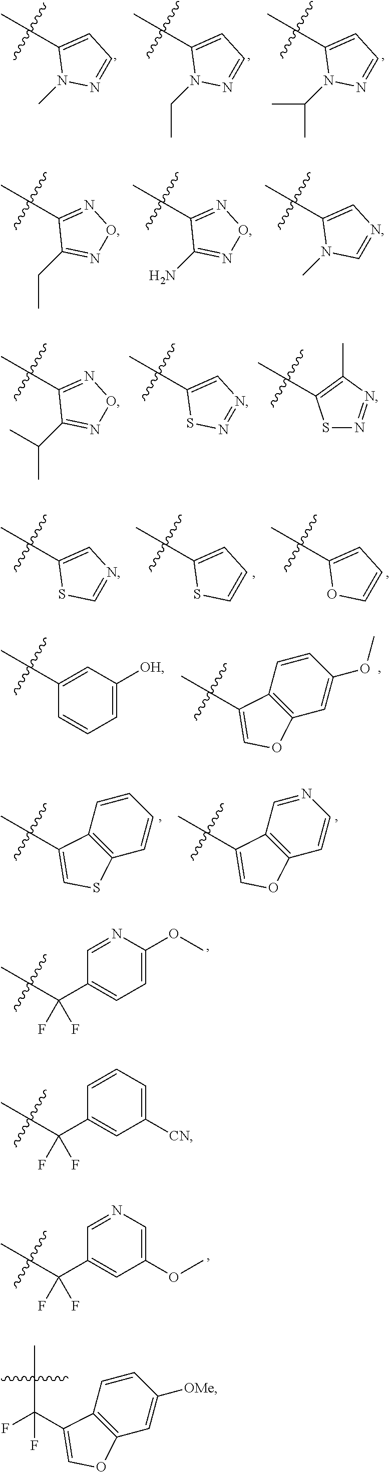

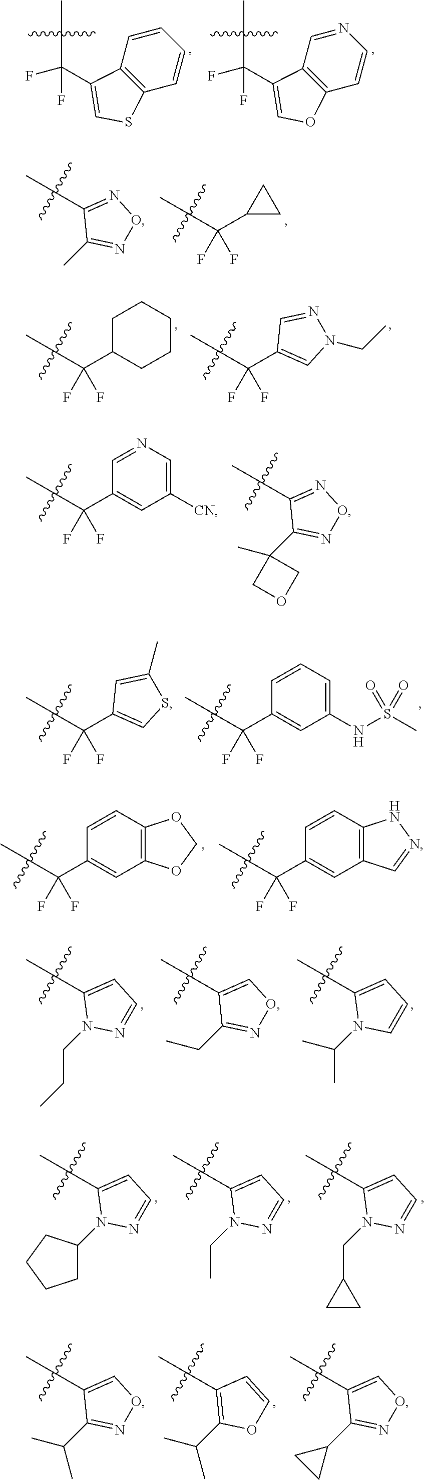

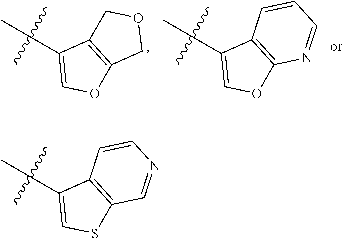 [SMILES: *c1coc2c1COC2 ; *c1coc2ncccc12 ; *c1csc2cnccc12]

wherein each optional substituent for the above Formula is independently selected from halogen, —OR^{F12}, —SR^{F12}, —N(R^{F12})₂, —C(O)R^{F12}, —C(O)N(R^{F12})₂, N(R^{F12})C(O)R^{F12}, —C(O)OR^{F12}, —OC(O)R^{F12}, —S(O)R^{F12}, —S(O)₂R^{F12}, —NO₂, ═O, ═S, ═N(R^{F12}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F12}, —N(R^{F12})₂, —C(O)R^{F12}, —C(O)N(R^{F12})₂, —N(R^{F12})C(O)R^{F12}, —C(O)OR^{F12}, —OC(O)R^{F12}, —NO, ═O, ═N(R^{F11}) and —CN.

In certain embodiments the I-17 Targeting Ligand is of Formula:

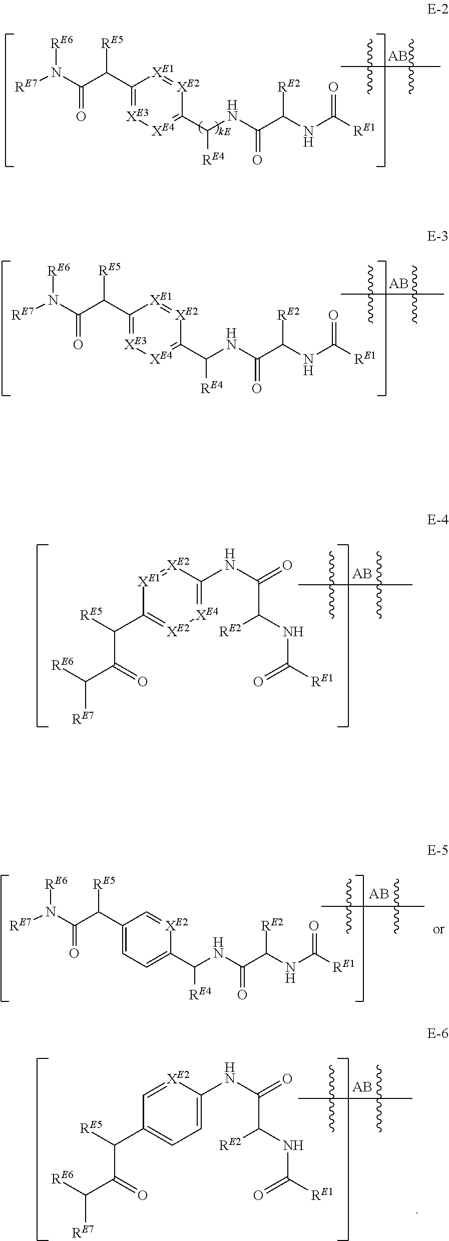

In certain embodiments IL-17 Targeting Ligand is of Formula:

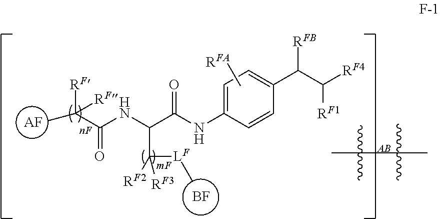

wherein,

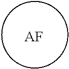

is selected from an optionally substituted C₃₋₁₂ carbocycle and optionally substituted 3- to 12-membered heterocycle wherein substituents on Ring AF are independently selected at each occurrence from:
halogen, —OR^{F11}, —SR^{F11}, —N(R^{F11})₂, —C(O)R^{F11}, —C(O)N(R^{F11})₂, N(R^{F11})C(O)R^{F11}, —N(R^{F11})S(O)₂R^{F11}, —C(O)OR^{F11}, OC(O)R^{F11}, S(O)R^{F11}, S(O)₂R^{F11}, —NO₂, ═O, ═S, ═N(R^{F11}), —CN; and
C₁₋₁₀ alkyl, C₂₋₁₀ alkenyl, C₂₋₁₀ alkynyl, each of which is optionally substituted with one or more substituents independently selected from halogen, —OR^{F11}, —SR^{F11}, —N(R^{F11})₂, —C(O)R^{F11}, —C(O)N(R^{F11})₂, —N(R^{F11})C(O)R^{F11}, —C(O)OR^{F11}, —OC(O)R^{F11}, —S(O)R^{F11}, —S(O)₂R^{F11}, —NO₂, ═O, ═S, ═N(R^{F11}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F11}, —N(R^{F11})₂, —C(O)R^{F11}, —C(O)N(R^{F11})₂, —N(R^{F11})C(O)R^{F11}, —C(O)OR11, —OC(O)R11, —NO 2, ═O═N(R11 and —CN′ and
C₃₋₁₂ carbocycle and 3- to 12-membered heterocycle, each of which is optionally substituted with one or more substituents independently selected from: halogen, —OR^{F11}, —SR^{F11}, —N(R^{F11})₂, —C(O)R^{F11}, —C(O)N(R^{F11})₂, N(R^{F11})C(O)R^{F11}, —C(O)OR^{F11}, —OC(O)R^{F11}, —NO₂, —CN, C₁₋₆ alkyl and C₁₋₆ haloalkyl;

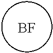

is selected from an optionally substituted C₃₋₁₀ carbocycle and optionally substituted 3- to 12-membered heterocycle each substituent on Ring B are independently selected at each occurrence from:
halogen, —ORF12, —SRF12, —N(RF12)2, —C(O)RF12, —C(O)N(RF12)2, —N(RF12)C(O)RF12, —C(O)ORF12, —OC(O)RF12, —S(O)RF12, —S(O)₂RF12, —NO2, ═O, ═S, ═N(RF12), —CN; and
C₁₋₁₀ alkyl, C₂₋₁₀ alkenyl, C₂₋₁₀ alkynyl, each of which is optionally substituted with one or more substituents independently selected from halogen, —OR^{F12}, —SR^{F12}, —N(R^{F12})₂, —C(O)R^{F12}, —C(O)N(R^{F12})₂, N(R^{F12})C(O)R^{F12}, —C(O)OR^{F12}, —OC(O)R^{F12}, —S(O)R^{F12}, —S(O)₂R^{F12}, —NO₂, ═O, ═S, ═N(R^{F12}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F12}, —N(R^{F12})₂, —C(O)R^{F12}, —C(O)N(R^{F12})₂, —N(R^{F12})C(O)R^{F12}, —C(O)OR^{F12}, —OC(O)R^{F12}, —NO, ═O, ═N(R^{F11}) and —CN;
R^{F4} is selected from —C(O)N(R^{F23})(R^{F24}) and C(O)heterocycle, wherein heterocycle is optionally substituted with 1, 2, 3, or 4 substituents selected from halogen, —OR^{F3}, —SR^{F3}, —N(R^{F13})₂, —C(O)R^{F13}, —C(O)N(R^{F13})₂, —N(R^{F13})C(O)R^{F13}, —C(O)OR^{F13}, —OC(O)R^{F13}, —S(O)R^{F13}, —S(O)₂R^{F13}, —NO₂, ═O, ═S, ═N(R^{F13})—CN; and
C₁₋₁₀ alkyl, C₂₋₁₀ alkenyl, C₂₋₁₀ alkynyl, each of which is optionally substituted with one or more substituents independently selected from halogen, —OR^{F13}, —SR^{F13}, —N(R^{F13})₂, —C(O)R^{F13}, —C(O)N(R^{F13})₂, N(R^{F13})C(O)R^{F13}, —C(O)OR^{F13}, —OC(O)R^{F13}, —S(O)R^{F13}, —S(O)₂R^{F13}, —NO₂, ═O, ═S, ═N(R^{F13}), —CN, C₃₋₁₀ carbocycle and 3- to 10-membered heterocycle, wherein the C₃₋₁₀ carbocycle and 3- to 10-membered heterocycle are each optionally substituted with one or more substituents selected from: halogen, —OR^{F13}, —N(R^{F13})₂, —C(O)R^{F13}, —C(O)N(R^{F13})₂, —N(R^{F13})C(O)R^{F13}, —C(O)OR^{F13}, —OC(O)R^{F13}, —NO₂, ═O, ═N(R^{F13}), and —CN;
L^{F} is bond or selected from —O— and —NH—;
R^{FA} is selected from hydrogen, halogen, —OR^{F14}, —N(R^{F14})₂, —C(O)R^{F14}, —C(O)N(R^{F14})₂, N(R^{F14})C(O)R^{F14}, —C(O)OR^{F14}, —OC(O)R^{F14}, —NO₂, —CN, and C₁₋₆ alkyl, wherein C₁₋₆ alkyl is optionally substituted with one or more substituents selected from: halogen, OR^{F14}, —N(R^{F14})₂, —C(O)R^{F14}, NO₂, ═O, and —CN;
R^{FB} is selected from hydrogen, halogen, —OR^{F15}, —N(R^{F15})₂, —C(O)R^{F15}, —C(O)N(R^{F15})₂, N(R^{F15})C(O)R^{F15}, —C(O)OR^{F15}, —OC(O)R^{F15}, —NO₂, —CN, and C₁₋₆ alkyl, wherein C₁₋₆ alkyl is optionally substituted with one or more substituents selected from: halogen, OR^{F15}, —N(R^{F15})₂, —C(O)R^{F15}, NO₂, ═O, and —CN, wherein at least one of R^{A} or R^{B} is not hydrogen;
R^{F}′ and R^{F}″ are independently selected from: hydrogen, halogen, —OR^{F16}, and C₁₋₆ alkyl; wherein the C₁₋₆ alkyl is optionally substituted with one or more substituents selected from: halogen, —OR^{F16}, —N(R^{F16})₂, —C(O)R^{F16}, —NO₂, ═O, and —CN;
R^{F1} is selected from —OR^{F21}, —N(R^{F21})(R^{F22}), —N(R^{F21})C(O)R^{F22}, —N(R^{F21})C(O)OR^{F22}, —N(R^{F21})C(O)N(R^{F21})(R^{F22}), —N(R^{F21})S(═O)₂N(R^{F21})(R^{F22}) and —N(R^{F21})S(═O)₂(R^{F22});
each R^{F2} and R^{F3} are independently selected from: hydrogen, halogen, —OR^{F17}, C₁₋₆ alkyl, and C₃₋₆ cycloalkyl; wherein the C₁₋₆ alkyl and C₃₋₆ cycloalkyl are optionally substituted with one or more substituents selected from: halogen, —OR^{F17}, —N(R^{F17})₂, —C(O)R^{F17}, —NO₂, ═O, and —CN; or
R^{F2} and R^{F3} bound to the same carbon come together to form a C₃₋₆ cycloalkyl optionally substituted with one or more substituents selected from halogen, —OR^{F17}, —N(R^{F17})₂, —C(O)R^{F17}, —NO₂, ═O, and —CN;
R^{F21} is independently selected at each occurrence from hydrogen and C₁-C₆ alkyl optionally substituted by one or more substituents independently selected from halogen, —OR^{F17}, —N(R^{F17})₂, —C(O)R^{F17}, —NO₂, ═O, and —CN;
R^{F22} is selected from: C₁₋₁₀ alkyl, C₂₋₁₀ alkenyl, C₂₋₁₀ alkynyl, each of which is optionally substituted with one or more substituents independently selected from halogen, —OR^{F8}, —SR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —S(O)R^{F18}, —S(O)₂R^{F18}, —NO₂, ═O, ═S, ═N(R^{F18}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —NO₂, ═O, ═N(R^{F18}), and —CN; and C₃₋₁₂ carbocycle and 3- to I2-membered heterocycle, each of which is optionally substituted with one or more substituents independently selected from:
halogen, —OR^{F18}, —SR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —S(O)R^{F18}, —S(O)₂R^{F18}, —NO₂, ═O, ═S, ═N(R^{F18}), —CN; and
C₁₋₁₀ alkyl, C₂₋₁₀ alkenyl, C₂₋₁₀ alkynyl, each of which is optionally substituted with one or more substituents independently selected from halogen, —OR^{F18}, —SR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —S(O)R^{F18}, —S(O)₂R^{F18}, —NO₂, ═O, ═S, ═N(R^{F18}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —NO═O, ═N(R^{F18}), and —CN; and
C₃₋₁₀ carbocycle and 3- to 10-membered heterocycle are each optionally substituted with one or more substituents selected from: halogen, —OR^{F18}, —N(R^{F18})₂, —C(O)R^{F18}, —C(O)N(R^{F18})₂, N(R^{F18})C(O)R^{F18}, —C(O)OR^{F18}, —OC(O)R^{F18}, —NO₂, ═O, ═N(R^{F18}), and —CN;
R^{F23} is selected from:
C₁₋₆ alkyl optionally substituted with one or more substituents independently selected from halogen, —OR^{F19}, —SR^{F19}, —N(R^{F19})₂, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F19}, —N(R^{F19})₂, ═O, C₁-C₆ alkyl, C₁-C₆ haloalkyl, and —CN; and
C₃₋₁₂ carbocycle and 3- to 10-membered heterocycle, each of which is optionally substituted with one or more substituents independently selected from halogen, —OR^{F19}, —N(R^{F19})₂, ═O, C₁-C₆ alkyl, C₁-C₆ haloalkyl, and —CN;
R^{F24} is selected from hydrogen and C₁₋₆ alkyl optionally substituted with one or more substituents independently selected from halogen, —OR^{F19}, SR^{F19}, —N(R^{F19})₂, —NO₂, —CN, C₃₋₆ carbocycle and 3- to 6-membered heterocycle;
R^{F11}, R^{F12}, R^{F13}, R^{F14}, R^{F15}, R^{F16}, R^{F17}, R^{F18}, and R^{F19} are independently selected at each occurrence from
hydrogen; and
C₁₋₆ alkyl optionally substituted with one or more substituents independently selected from halogen, —OH, —O—C₁-C₆ alkyl, —O—C₁-C₆haloalkyl —NH2, —NO2, ═O, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OH, —O—C₁-C₆ alkyl, —O—C₁-C₆ haloalkyl —NH₂, —NO₂, ═O, and —CN; and
C₃₋₁₂ carbocycle and 3- to 12-membered heterocycle, each of which is optionally substituted with one or more substituents independently selected from:
halogen, —OH, —O—C₁-C₆ alkyl, —O—C₁-C₆haloalkyl —NH2, —NO₂, ═O, —CN; and
C₁₋₆ alkyl optionally substituted with one or more substituents independently selected from halogen, —OH, —O—C₁-C₆ alkyl, —O—C₁-C₆ haloalkyl, —NH₂, —NO₂, ═O, and —CN;
n^{F} is selected from 0 and 1; and
m^{F} is selected from 0, 1, and 2.

In certain embodiments IL-17 Targeting Ligand is of Formula:

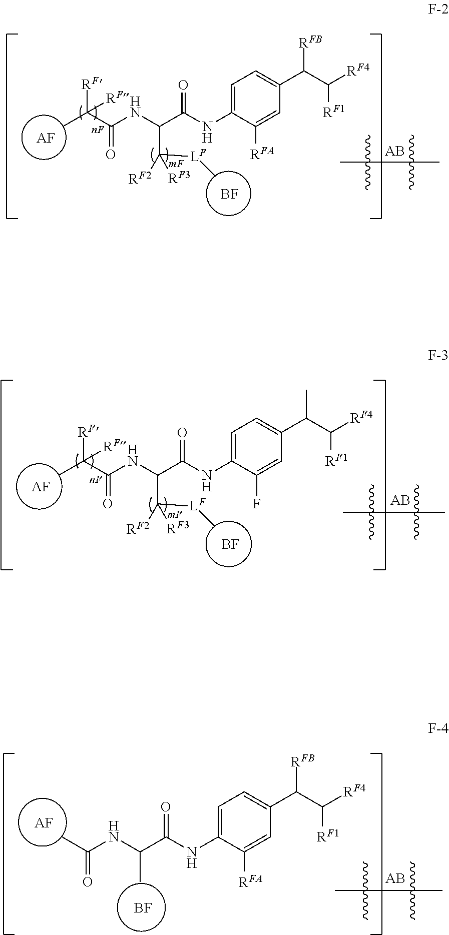

In certain embodiments IL-17 Targeting Ligand is of Formula:

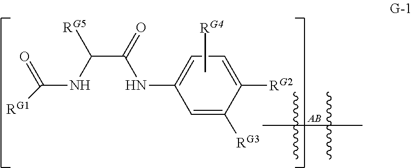

wherein,
RG1 is selected from the group consisting of 5- or 6-membered heteroaryl, 9- or 10-membered bicyclic heteroaryl, phenyl, (C₁-C₆)alkoxy, (C₃-C₇)cycloalkoxy, (C₁-C₆)alkyl, phenyl-(C₁-C₄)alkyl, (C₃-C₇)cycloalkyl, 4-6-membered heterocycloalkyl and —NR^{GC}R^{GD}, wherein said 5- or 6-membered heteroaryl, 9- or 10-membered bicyclic heteroaryl, phenyl, (C₁-C₆)alkoxy, (C₃-C₇)cycloalkoxy, (C₁-C₆)alkyl, phenyl-(C₁-C₄)alkyl, (C₃-C₇)cycloalkyl and 4-6-membered heterocycloalkyl is optionally substituted with one or more substituents independently selected from R^{GA};
R^{GA} represents deuterium, halogen, hydroxy, —NR^{GC}R^{GD}, (C₁-C₆)alkyl, (C₁-C₆)alkylcarbonyl, (C₃-C₇)cycloalkyl, phenyl, 5- or 6-membered heteroaryl or, 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, —SO₂—(C₁-C₄)alkyl and —NR^{GC}R^{GD};
R^{G2} is selected from the group consisting of 5- or 6-membered heteroaryl, wherein said 5- or 6-membered heteroaryl is optionally substituted with one or more substituents independently selected from R^{GB}, wherein said 5- or 6-membered heteroaryl may optionally contain —CO— as a ring member and wherein when said 5 membered heteroaryl contains nitrogen as a ring atom said nitrogen may optionally be substituted with a substituent selected from R^{G8};
R^{GB} represents deuterium, halogen, cyano, hydroxy, —NR^{GC}R^{GD}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR^{GC}R^{GD} and (C₁-C₄)alkoxy;
R^{GC} and R^{GD} each independently are selected from the group consisting of hydrogen and (C₁-C₆)alkyl, or R^{GC} and R^{GD} together form pyrrolidinyl or piperidinyl, wherein said (C₁-C₆)alkyl, pyrrolidinyl or piperidinyl is optionally substituted with one or more substituents independently selected from halogen, cyano and hydroxy;
R^{G8} is selected from the group consisting of -L^{G}-PO(OH)₂ and —CHR^{GG}O—(CO-A-NR^{GH}))_{0 or 1)}—CO-A-NR^{GH}R^{GI};
L^{G} is selected from the group consisting of a bond or —CHR^{GG}O—;
wherein each —CO-A-NR^{GH}— independently represents an amino acid residue wherein the amino acid residue is selected from the natural amino acids either in D or L-form or as mixtures of the D and L form, and wherein said amino acid residue may be substituted on the a-amino group with a substituent R^{GH};
R^{GG}, R^{GH}, and R^{GI} are independently selected from hydrogen and (C₁-C₆) alkyl;
R^{G3} is selected from the group consisting of hydrogen, deuterium, hydroxy and halogen;
R^{G4} is selected from the group consisting of hydrogen, deuterium and halogen;
R^{G5} is selected from the group consisting of —CHR^{G6}R^{G7}, (C₃-C₁₀)cycloalkyl and GG, wherein said (C₃-C₁₀)cycloalkyl and GG are optionally substituted with one or more substituents independently selected from deuterium, halogen, cyano, hydroxy, (C₁-C₄)alkyl and halo(C₁-C₄)alkyl;
GG represents

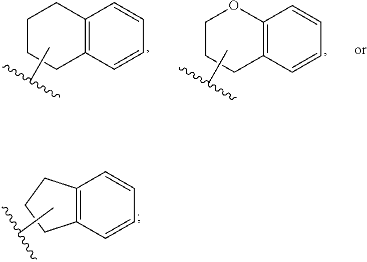

and
R^{G6} and R^{G7} each independently represents hydrogen, phenyl, (C₁-C₆)alkyl, or (C₃-C₇)cycloalkyl, wherein said phenyl, (C₁-C₆)alkyl or (C₃-C₇)cycloalkyl is optionally substituted with one or more substituents independently selected from halogen, cyano, hydroxy and (C₁-C₄)alkyl.

In certain embodiments IL-17 Targeting Ligand is of Formula:

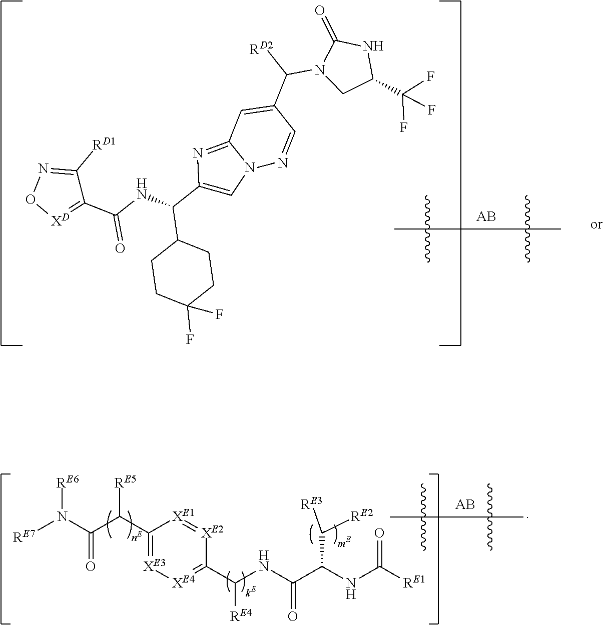

### Interleukin-6 (IL-6)

In some embodiments, the Target Extracellular Protein is human interleukin-6 (IL-6) (UniProtKB-P05231 (IL6_HUMAN)). IL-6 is a cytokine with a wide variety of biological functions. It is a potent inducer of the acute phase response and plays an essential role in the final differentiation of B-cells into Ig-secreting cells. It is also involved in lymphocyte and monocyte differentiation. It also acts on B-cells, T-cells, hepatocytes, hematopoietic progenitor cells and cells of the CNS, and is required for the generation of T(H)17 cells. IL-6 has been implicated in a number of inflammatory diseases and cancers, including, but not limited to, Castleman's disease, metastatic castration-associated prostate cancer, renal cell carcinoma, large-cell lung carcinoma, ovarian cancer, rheumatoid arthritis, asthma.

The Protein Data Bank website provides the crystal structure of IL-6 searchable by 1P9M (Boulanger, M. J., et al., Science, 2003, 300: 2101-2104); IALU (Somers et al., EMBO J., 1997, 16, 989-997); 1IL6 and 2IL6 (Xu, G. Y., et al., J Mol Biol., 1997, 268 468-481) and 1N26 (Varghese et al., Proc Natl Acad Sci USA., 2002, 99 15959-15964); as well as the crystal structure of IL-6 bound to various compounds searchable by 4CNI (Shaw, S., et al., Mabs, 2014, 6: 773); and 4NI7 and 4NI9 (Gelinas et al., J Biol Chem. 2014, 289(12), 8720-8734). Additionally, Gelinas et al., provides insight into the crystal structure of interleukin-6 in complex with a modified nucleic acid ligand (Gelinas, A. D., et al., J Biol Chem. 2014, 289(12), 8720-8734); and Somers et al., provides insight into the crystal structure of interleukin 6: implications for a novel mode of receptor dimerization and signaling.

Non-limiting examples of IL-6 direct or indirect inhibitors are provided in FIG. 1. Additional IL-6 direct or indirect inhibitors can be found in, for example, U.S. Pat. No. 8,901,310; U.S. patent Ser. No. 10/189,796; U.S. Pat. No. 9,694,015; each incorporated herein by reference. In another embodiment the IL-6 Extracellular Targeting Ligand is AvimarC326 or a binding fragment thereof which is described in Nat Biotechnol 23, 1556-1561 (2005).

In some embodiments, the Target Extracellular Protein is Interleukin-6. Interleukin-6 (IL-6) is a cytokine that is a crucial component of the acute phase immune response. IL-6 ligand binds to IL-6 receptor, and the heterodimer then associates with IL6ST and gp130, stimulating a response. During infection certain molecules from pathogens bind to toll-like receptors, which activate macrophages to produce IL-6. In addition to stimulating differentiation of B cells and neutrophils, IL-6 mediates the fever response.

IL-6 has been implicated in many inflammatory diseases, including multiple sclerodid, neuromyelitis optica spectrum disorder, diabetes, atherosclerosis, depression, Alzheimer's disease, systemic lupus erythromatosus, multiple myeloma, prostate cancer, Behcet's disease, rheumatoid arthritis, systemic juvenile idiopathic arthritis, and Castleman's disease.

IL-6 signaling is also important to the musculoskeletal system. In bone, it interacts with VEGF stimulating angiogenesis. In muscle cells, IL-6 is produced in large amounts during exercise. In contrast to its role in stimulating the immune system, during exercise IL-6 is anti-inflammatory.

The Protein Data Bank website provides the crystal structure of Interleukin-6, searchable by 1ALU (Somers, W. S. et al. 1.9 A crystal structure of interleukin 6: implications for a novel mode of receptor dimerization and signaling. (1997) EMBO J. 16: 989-997), 1IL6 (Xu, G. Y. et al. Solution structure of recombinant human interleukin-6 (1997) J Mol Biol 268: 468-481), and the structure of IL-6 bound in the active hexameric complex searchable by 1P9M (Boulanger, M. J. et al. Hexameric Structure and Assembly of the Interleukin-6/IL-6-alpha-Receptor/gp130 Complex. (2003) Science 300: 2101-2104)

Non-limiting examples of IL-6 Targeting Ligands can be found in, for example, USP 10633423, USP 10669314, US 2004/0092720, and Ranganath, S. et al. Discovery and Characterization of a Potent Interleukin-6 Binding Peptide with Neutralizing Activity In Vivo. PLoS ONE 10(11):e0141330.

SEQ ID NO: 380
QSDChaDCIHRLLEAF(4-F)LDPNLTEEQRWEKI
GlaKINDECE
(Ranganath, S. et al. PLoS ONE 10(11):
e0141330)
SEQ ID NO: 381
QSDChaDCIHRLLEAF(4-F)LDPNLTEEQRWERIGlaK
(PEG30L)INDECE
(Ranganath, S. et al. PLoS ONE 10(11):
e0141330)
SEQ ID NO: 382
QSDChaDCIHRLLEAF(4-F)LDPNL
TEEQRWERIGlaK(PEG20Br)INDECE
(Ranganath, S. et al. PLoS ONE 10(11):
e0141330)
SEQ ID NO: 383
QSDChaDCIHRLLEAF(4-F)LDP
NLTEEQRWERIGlaK(PEG40Br)INDECE
(Ranganath, S. et al. PLoS ONE 10(11):
e0141330)
SEQ ID NO: 384
FDhLDCIHRLLEAFLDPNLTEQQRWEKIDKINDECE
(Ranganath, S. et al. PLoS ONE 10(11):
e0141330)
SEQ ID NO: 385
QSDChaDCIHRLLEAF(4-F)LDPNLTEEQRWERIGla
KINDECE
(Ranganath, S. et al. PLoS ONE 10(11):
e0141330)
SEQ ID NO: 386
SWQSDChaDCIHRLLEAFLDK-AcNLTEEQRWERIDKIN
DECE
(Ranganath, S. et al. PLoS ONE 10(11):
e0141330)
SEQ ID NO: 387
SWQSDChaDCIHRLLEAFLDK-(PEG40Br)-
NLTEEQRWERIDKINDECE
(Ranganath, S. et al. PLoS ONE 10(11):
e0141330)

In certain embodiments the IL-6 Targeting ligand is SEQ ID NO: 388, bound to the linker through the PEGylated lysine residue.

SEQ ID NO: 388
EEX₃X₄AWX₇EIHX₁₁LPNLX₁₆X₁₇X₁₈QX₂₀X₂₁AFIX₂₅X₂₆LX₂₈X₂₉
(U.S. Pat. No. 10,633,423)

wherein, independently from each other,
X₃ is selected from A, F, H, K, Q, R, S, W and Y;
X₄ is selected from A, D, F, F, H, I, K, L, M, N, Q, R, S, T, V and Y;
X₇ is selected from F, H, I, K, L, M, N, R, S, T, V, W and Y;
X₁₁ is selected from A, I, K, L, M, N, R, S, T and V;
X₁₆ is selected from N and T;
X₁₇ is selected from A, I, T and V;
X₁₈ is selected from D, E, G, H, K, N, Q, R, S and T;
X₂₀ is selected from I, L, M, R, T and V;
X₂₁ is selected from A, S, T and V;
X₂₆ is selected from I, M, Q, S, T, V and W;
X₂₆ is selected from K and S,
X₂₈ is selected from F, L, M and Y; and
X₂₉ is selected from D and R;

SEQ ID NO: 389
EEX₃X₄AWX₇EIHX₁₁LPNLX₁₆X₁₇X₁₈QX₂₀X₂₁AFIX₂₅X₂₆LX₂₈X₂₉
(U.S. Pat. No. 10,669,314)

wherein, independently from each other,
X₃ is selected from A, F, H, K, Q, R, S, W and Y;
X₄ is selected from A, D, E, F, H, I, K, L, M, N, Q, R, S, T, V and Y;
X₇ is selected from F, H, I, K, L, M, N, R, S, T, V, W and Y;
X₁₁ is selected from A, I, K, L, M, N, R, S, T and V;
X₁₆ is selected from N and T;
X₁₇ is selected from A, I, T and V;
X₁₈ is selected from D, E G, H, K, N, Q, R, S and T;
X₂₀ is selected from I, L, M, R, T and V;
X₂₁ is selected from A, S, T and V;
X₂₅ is selected from I, M, Q, S, T, V and W;
X₂₆ is selected from K and S;
X₂₈ is selected front F, L, M and Y; and
X₂₉ is selected from D and R;

In certain embodiments the targeting ligand for treating an IL-6 mediated disease binds to gp130. Non-limiting examples of gp130 Targeting Ligands can be found in, for example, Ahn, S-H. et al. In vitro and in vivo pharmacokinetic characterization of LMT-28 as a novel small molecular interleukin-6 inhibitor 2020 Asian-Australas J Anim Sci. 33:670-677, Aqel, S. I. Novel small molecule IL-6 inhibitor suppresses autoreactive Th17 development and promotes Treg development. (2019) Clinical and Experimental Immunology, 196:215-225, Hong, S.-S. et al. A Novel Small-Molecule Inhibitor Targeting the IL-6 Receptor beta Subunit, Glycoprotein 130. 2015 J Immunol 195:237-245

In certain embodiments the gp130 binding Targeting Ligand is selected from

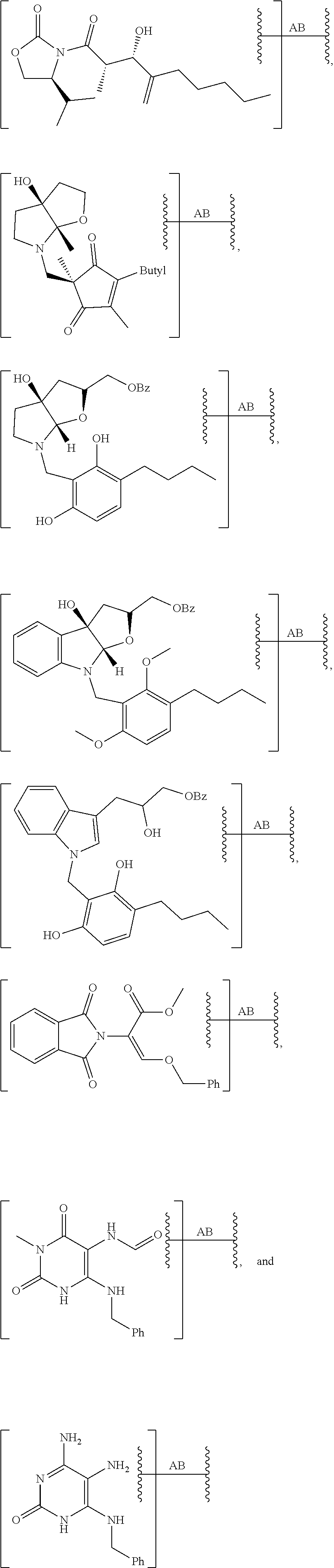

### Immunoglobulin A1 (IgA1)

Immunoglobulin A is a class of antibodies which is commonly found in secretions, but is also present in serum. IgA contains four heavy chains and four light chains, in a dimeric form. IgA exists in two isotypes, IgA1 and IgA2. IgA1 contains more repeats in the hinge region and is the predominant form found in serum. While production of IgA maintains strong mucosal immunity and defending against pathogens, it can become toxic. IgA nephropathy, also known as Berger's disease, is the pathological buildup of IgA antibodies which reduces kidney function. The etiology of the disease remains unclear, however it has been suggested that the glycosylation pattern on the hinge region plays a role. As proper kidney function is important for overall health, IgA nephropathy is associated with systemic diseases such as liver failure, cancer, celiac disease, systemic lupus erythematosus, rheumatoid arthritis, heart failure, reactive arthritis, and ankylosing spondylitis.

The Protein Data Bank website provides the crystal structure of IgA1, and representative example include PDB accession codes 1IGA (Boehm, M. K. 1999, J. Mol. Bio. 286 1421-1447), 2ESG (Almogren, A. 2006 J. Mol. Biol. 356, 413-431), 6XJA, 7JGJ, (Eisenmesser, E. Z. 2020, Nat. Commun, 11, 6063-6063), and 3CHN (Bonner, A. 2009, Mucosal Immunol., 2, 74-84).

Direct or indirect IgA1 binding molecules include jacalin and SEQ ID NO: 390

YYALSDAKEEEPRYKALRGENQDLRE
KERKYQDKIKKLEEKEKNLEKKS.

### VI. Pharmaceutical Compositions and Dosage Forms for the Extracellular Degrading Compounds of the Present Invention

A compound of the present invention or a pharmaceutically acceptable salt, solvate or prodrug thereof as disclosed herein can be administered as a neat chemical, but is more typically administered as a pharmaceutical composition that includes an effective amount for a host, typically a human, in need of such treatment to treat a disorder mediated by the target extracellular protein, as described herein or otherwise well-known for that extracellular protein.

The Extracellular Protein degraders of the present invention can be administered in any manner that allows the degrader to bind to the Extracellular Protein, typically in the blood stream, and carry it to the ASGPR or mannose 6-phosphate receptor bearing cells for endocytosis and degradation. As such, examples of methods to deliver the degraders of the present invention include, but are not limited to, oral, intravenous, sublingual, subcutaneous, parenteral, buccal, rectal, intra-aortal, intracranial, subdermal, transdermal, controlled drug delivery, intramuscular, or transnasal, or by other means, in dosage unit formulations containing one or more conventional pharmaceutically acceptable carriers, as appropriate. In certain embodiments, the degrader is provided in a liquid dosage form, a solid dosage form, a gel, particle, etc.

In certain embodiments the compound of the present invention is administered subcutaneously. Typically, the compound will be formulated in a liquid dosage form for subcutaneous injection, such as a buffered solution. Non-limiting examples of solutions for subcutaneous injection include phosphate buffered solution and saline buffered solution. In certain embodiments the solution is buffered with multiple salts.

In certain embodiments the compound of the present invention is administered intravaneously. Typically, the compound will be formulated in a liquid dosage form for intravaneous injection, such as a buffered solution. Non-limiting examples of solutions for intravaneous injection include phosphate buffered solution and saline buffered solution. In certain embodiments the solution is buffered with multiple salts.

Therefore, the disclosure provides pharmaceutical compositions comprising an effective amount of degrading compound or its pharmaceutically acceptable salt together with at least one pharmaceutically acceptable carrier for any appropriate use thereof. The pharmaceutical composition may contain a compound or salt as the only active agent, or, in an alternative embodiment, the compound and at least one additional active agent.

The term “pharmaceutically acceptable salt” as used herein refers to a salt of the described compound which is, within the scope of sound medical judgment, suitable for administration to a host such as a human without undue toxicity, irritation, allergic response, and the like, commensurate with a reasonable benefit/risk ratio, and effective for its intended use. Thus, the term “pharmaceutically acceptable salt” refers to the relatively non-toxic, inorganic and organic acid addition salts of the presently disclosed compounds. These salts can be prepared during the final isolation and purification of the compounds or by separately reacting the purified compound in its free form with a suitable organic or inorganic acid and then isolating the salt thus formed. Basic compounds are capable of forming a wide variety of different salts with various inorganic and organic acids. Acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents.

Pharmaceutically acceptable base addition salts may be formed with a metal or amine, such as alkali and alkaline earth metal hydroxide, or an organic amine. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Any dosage form can be used that achieves the desired results. In certain embodiments the pharmaceutical composition is in a dosage form that contains from about 0.1 mg to about 1500 mg, from about 10 mg to about 1000 mg, from about 100 mg to about 800 mg, or from about 200 mg to about 600 mg of the active compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 100, 200, 250, 300, 400, 500, 600, 700, or 750 mg of active compound, or its salt.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In some embodiments, compounds disclosed herein or used as described are administered once a day (QD), twice a day (BID), or three times a day (TID). In some embodiments, compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the compound of the present invention is administered once a day, twice a day, three times a day, or four times a day.

The pharmaceutical composition may be formulated as any pharmaceutically useful form, e.g., a pill, capsule, tablet, an injection or infusion solution, a syrup, an inhalation formulation, a suppository, a buccal or sublingual formulation, a parenteral formulation, or in a medical device. Some dosage forms, such as tablets and capsules, can be subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution (HA, 5 mg/mL in PBS), citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Suitable buffers are well known by those skilled in the art. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions/combinations can be formulated for oral administration. These compositions can contain any amount of active compound that achieves the desired result, for example between 0.1 and 99 weight % (wt. %) of the compound and usually at least about 5 wt. % of the compound. Some embodiments contain from about 25 wt. % to about 50 wt. % or from about 5 wt. % to about 75 wt. % of the compound. Enteric coated oral tablets may also be used to enhance bioavailability of the compounds for an oral route of administration.

Formulations suitable for rectal administration are typically presented as unit dose suppositories. These may be prepared by admixing the active compound with one or more conventional solid carriers, for example, cocoa butter, and then shaping the resulting mixture.

### VII. Treatment of Diseases with the Disclosed Extracellular Protein Degraders

The Target Proteins of the current invention may include, but are not limited to, immunoglobulins, cytokines, chemokines, growth factors, coagulation factors, extracellular matrix proteins and proteins involved in formation and/or degradation of the extracellular matrix, esterases, lipases, peptidases, convertases, among others. These proteins mediate a range of diseases that can be treated with an effective amount of the disclosed Extracellular Protein Degraders described herein.

### Immunoglobulins

1) Immunoglobulin A (IgA) aberrant expression mediates a range of autoimmune and immune-mediated disorders, including IgA nephropathy (also known as Berger's disease), celiac disease, Crohn's disease, Henoch-Sconiein purpura (HSP) (also known as IgA vasculitis), liner IgA bullous dermatosis, IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, α-chain disease, IgA monoclonal gammopathy, monoclonal gammopathy of undetermined significance (MGUS), and linear IgA bullous dermatosis, among others.
2) Immunoglobulin G (IgG) mediates a range of autoimmune, infectious and metabolic diseases, including systemic fibroinflammatory disease. In addition, overexpression of IgG4 is associated with IgG4-related diseases, which generally include multiple organs, and disorders include type 1 autoimmune pancreatitis, interstitial nephritis, Riedel's thyroiditis, storiform fibrosis, Mikulicz's disease, Kuttner's tumor, inflammatory pseudotumors (in various sites of the body), mediastinal fibrosis, retroperitoneal fibrosis (Ormond's disease), aortitis and periaortitis, proximal biliary strictures, idiopathic hypocomplementic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenic gravis, ankylosing spondylitis, primary Sjögren's syndrome, psoriatic arthritis, and systemic lupus erythematosus (SLE), sclerosing cholangitis, and IgG monoclonal gammopathy, monoclonal gammopathy of undetermined significance (MGUS), among others.
3) Immunoglobulin E (IgE)—IgE is a strong mediator of allergic disease, including but not limited to, atopic asthma, allergic rhinitis, atopic dermatitis, IgE-mediated food allergy, IgE-mediated animal allergies, allergic conjunctivitis, allergic urticaria, anaphylactic shock, nasal polyposis, keratoconjunctivitis, mastocytosis, and eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, and monoclonal gammopathy of undetermined significance (MGUS), among others.

### Cytokines/Chemokines

1) TNF-α mediates a number of disorders, including but not limited to rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia.
2) IL-2 mediates host versus graft rejection in transplants and autoimmune disorders, including, but not limited to, multiple sclerosis, idiopathic arthritis, iritis, anterior uveitis, IL-2 induced hypotension, psoriasis, and other autoimmune disorders
3) IL-1 mediates a number of auto-inflammatory and autoimmune disorders, including, but not limited to, Blau syndrome, cryopyrin-associated periodic syndromes, familial Mediterranean fever, Majeed syndrome; mevalonate kinase deficiency syndrome, pyogenic arthritis-pyoderma gangrenosum-acne syndrome, tumor necrosis factor receptor-associated periodic syndrome, Behçet's Disease, Sjogren's Syndrome, gout and chondrocalcinosis, periodic fever, aphthous stomatitis, pharyngitis, and cervical adenitis (or PFAPA) syndrome, rheumatoid arthritis, Type 2 diabetes mellitus, acute pericarditis, Chronic interstitial lung diseases (ILDs), and Still's disease amongst others.
4) IFN-γ mediates a wide range of autoimmune disorders, including, but not limited to rheumatoid arthritis, multiple sclerosis (MS), corneal transplant rejection, and various autoimmune skin diseases such as psoriasis, alopecia areata, vitiligo, acne vulgaris, and others.
5) IL-21 mediates a number of autoimmune disorders, including Sjögren's syndrome, systemic lupus erythematosus, type 1 diabetes, multiple sclerosis, rheumatoid arthritis, and inflammatory bowel disease.
6) IL-22 mediates a number of autoimmune disorders, including, but not limited to, graft versus host disease (GVHD), psoriasis, rheumatoid arthritis, atopic dermatitis, and asthma.
7) IL-10 has been implicated in tumor survival and protection against cytotoxic chemotherapeutic drugs.
8) IL-5 has been implicated in a number of allergic disorders, including, but not limited to, asthma, nasal polyposis, atopic dermatitis, eosinophilic esophagitis, hypereosinophilic syndrome, and Churg-Strauss syndrome.
9) IL-6 has been implicated in a number of inflammatory diseases and cancers, including, but not limited to, Castleman's disease, metastatic castration-associated prostate cancer, renal cell carcinoma, large-cell lung carcinoma, ovarian cancer, rheumatoid arthritis, asthma.
10) IL-8 has been implicated in the promotion of tumor progression, immune escape, epithelial-mesenchymal transition, and recruitment of myeloid-derived suppressor cells. Studies have demonstrated that high serum IL-8 levels correlate with poor prognosis in many malignant tumors. Preclinical studies have shown that IL-8 blockade may reduce mesenchymal features in tumor cells, making them less resistant to treatment.
11) C-C motif chemokine ligand 2 (CCL2) has been implicated in the recruitment of monocytes into the arterial wall during the disease process of atherosclerosis.
12) Macrophage Migration Inhibitory Factor (MIF) is a mediator of tumor progression; systemic inflammation; atherosclerosis; rheumatoid arthritis; and systemic lupus erythematosus, among others.

### Growth Factors

1) Fibroblast Growth Factor 1 (FGF1) can induce angiogenesis. FGF1 has been implicated in oncogenesis, cancer cell proliferation, resistance to anticancer therapies, and neoangiogenesis.
2) Fibroblast Growth Factor 2 (FGF2) has been implicated in oncogenesis, cancer cell proliferation, resistance to anticancer therapies, and neoangiogenesis.
3) Vascular Epithelial Growth Factor (VEGF-A) has been implicated in the vascularization and angiogenesis of tumors.
4) Transforming Growth Factor-β1 (TGF-β1) expression in the tumor microenvironment has been associated with a poor prognosis, and is implicated in TGF-β1 mediated tumor suppression via T-cell exclusion. TGF-β1 expression has also been implicated in hematological malignancies and fibrosis.
5) Transforming Growth Factor-β2 (TGF-β2) expression in the tumor microenvironment has been associated with a poor prognosis, and is implicated in TGF-β2 mediated tumor suppression via T-cell exclusion. TGF-β2 expression has also been implicated in hematological malignancies and fibrosis.
6) Placental Growth Factor (PGF) promotes cell tumor growth, and has been implicated in age-related macular degeneration (AMD) and choroidal neovascularization (CNV).

### Esterase

1) Cholinesterase has been implicated in cognitive disorders such as dementia and Alzheimer's disease.

### Coagulation Factors

1) Carboxypeptidase B2 has been implicated and targeted to inhibit thrombosis.
2) Coagulation Factor Xa is a mediator in the development of deep vein thrombosis and acute pulmonary embolism, and the risk of stroke and embolism in people with nonvalvular atrial fibrillation.
3) Coagulation Factor XI is a mediator in the development of deep vein thrombosis and acute pulmonary embolism, and the risk of stroke and embolism in people with nonvalvular atrial fibrillation.
4) Coagulation Factor XII has been implicated in the development of deep vein thrombosis and acute pulmonary embolism, and the risk of stroke and embolism in people with nonvalvular atrial fibrillation.
5) Coagulation Factor XIII has been implicated in the development of deep vein thrombosis and acute pulmonary embolism, and the risk of stroke and embolism in people with nonvalvular atrial fibrillation.
6) Prothrombin is involved in blood clot formation and arterial and venous thrombosis, and thromboembolism associated with atrial fibrillation.
7) Coagulation Factor VII is involved in blood clot formation and arterial and venous thrombosis, and thromboembolism associated with atrial fibrillation.
8) Coagulation Factor IX is involved in blood clot formation and arterial and venous thrombosis, and thromboembolism associated with atrial fibrillation.

### Extracellular Matrix Proteins

1) Neutrophil Elastase—Neutrophil elastase has been implicated in a number of disorders, including lung disease, chronic obstructive pulmonary disease, pneumonia, respiratory distress, and acute lung injury (ALI), and cystic fibrosis, as well as chronic kidney disease.
2) Fibronectin-1—Interfering with FN polymerization may attenuate myofibroblasts and fibrosis and improve cardiac function after ischemia/reperfusion (I/R) injury.
3) Thrombospondin-1—TSP-1 has been implicated in a number of diseases, including in promoting certain cancers such as breast cancer, prostate cancer, melanoma, SCLC, osteosarcoma, cutaneous squamous cell carcinoma, oral squamous cell carcinoma, papillary thyroid carcinoma, thyroid cancer, medulloblastoma, and fibrotic disorders such as diabetes, liver fibrosis, and in multiple myeloma.
4) Urokinase-type Plasminogen Activator (UPA)—UPA has been implicated in vascular diseases and cancer progression. Elevated expression levels of urokinase and several other components of the plasminogen activation system are found to be correlated with tumor malignancy.
5) Plasminogen Activator, Tissue Type (TPA)—PLA has been shown activated in various cancers including oral malignancy.
6) Plasminogen (PLG)—PLG has been implicated in tumor invasion and inflammation.
7) Plasminogen Activator Inhibitor-1 (PAI-1)—PAI-1 has been implicated in angiogenesis, metastasis, and poor prognosis in tumors, including, but not limited to, oral cancers and breast cancers.

### Peptidase

1) Kallikrein-1—Kallikrein has been implicated in adverse reactions in hereditary angioedema (HAE).
2) Plasma Kallikrein—Plasma kallikrein has been implicated in retinal dysfunction, the development of diabetic macular edema and hereditary angioedema (HAE).
3) Matrix Metallopeptidase-1—MMP-1 has been implicated in cardiovascular disease, development of fibrosis, and growth of certain cancers such as bladder cancer.
4) Phospholipase A2, Group IIA (PA2GA)—PA2GA has been implicated in a number of diseases, including cardiovascular diseases, atherosclerosis, immune disorders, and cancer.

### Lipase

1) Lipoprotein Lipase—Lipoprotein lipase has been implicated in the development of cardiovascular disease and obesity.
2) Phospholipase A2, Group IB (PA21B)—PA21B has been implicated in a number of diseases, including cardiovascular diseases, atherosclerosis, immune disorders and cancer.

### Convertase

1) Proprotein Convertase Subtilisin/Kexin Type 9 (PCSK-9)—PCSK-9 has been implicated in high blood cholesterol and the development of cardiovascular disease.

Certain extracellular protein targets include but are not limited to: SAA (serum amyloid A), amyloid light chains, antibodies to Klebsiella dipeptidase protein; Ig antibodies to anionic phospholipids and beta-2-glycoprotein-I; IL-13; MIF; Transthyretin (misfolded), IgG autoantibodies to thyroid peroxidase, thyroglobulin and TSH receptors; TNF-α; Protein arginine deiminase (PAD, PAD4); antibodies to citrullinated protein antibody (ACPA); anti-DNA antibodies; IL-17; Lysyl Oxidase 2 (LOXL2); IL-18; Blys; B cell activating factor (BAFF); CD4O (soluble); CXCL12; soluble PSMA; matrix metalloproteinase IX (MMP-9); hormone-sensitive lipase; lipoprotein-associated phospholipase A2; Factor Xa; DPP4; thrombin; PCSK9; ApoB-100; Complement component C3b; PKK (pre-kallikrein); Factor Xi; PF4; Anti-vWF antibodies; anticardiolipin antibodies and lupus anticoagulant; FGF23 (fibroblast growth factor 23); Plasminogen activator inhibitor type I (PAI-1); Myeloperoxidase (MPO) extracellular; Myostatin; Beta2-m; suPAR (soluble urokinase plasminogen activator receptor); anti-ganglioside IgG; amyloid beta; Tau; CD-associate prion; anti-ganglioside IgG; HTT; anti-ganglioside IgG; synuclein; elastase; PABA (protective antigen of Bacillus anthracis); edema factor; Botulinum toxin; C. difficile toxin B; hemolysin; tetanus toxin; IL-2; growth hormone and ACTH.

### VIII. Exemplary Methods of Treatment of Diseases Mediated by Extracellular Proteins

The present invention can be used to treat any disorder that is mediated by the selected target disease-mediating extracellular protein. Nonlimiting examples of indications include autoimmune, other immune dysfunctions, complement mediated disorders, abnormal cellular proliferation, cancer, tumors, hematology-related disorders, renal disorders and liver disorders.

In certain embodiments, the degrader or its salt or composition as described herein is used in the treatment of an autoimmune disorder. In some aspects, the extracellular protein is an Ig, such as IgA or IgG. IgG degradation can treat for example, thyroid eye disease, myasthenia gravis, chronic inflammatory demyelinating polyneuropathy, and warm autoimmune hemolytic anemia.

Non-limiting examples of autoimmune disorders include: lupus, allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), diabetes, multiple sclerosis, pernicious anemia, psoriasis, rheumatoid arthritis, sarcoidosis, and scleroderma.

In an embodiment, the degrader or its salt or composition as described herein is used in the treatment of lupus. Non-limiting examples of lupus include lupus erythematosus, cutaneous lupus, discoid lupus erythematosus, chilblain lupus erythematosus, or lupus erythematosus-lichen planus overlap syndrome. Lupus erythematosus is a general category of disease that includes both systemic and cutaneous disorders. The systemic form of the disease can have cutaneous as well as systemic manifestations. However, there are also forms of the disease that are only cutaneous without systemic involvement. For example, SLE is an inflammatory disorder of unknown etiology that occurs predominantly in women, and is characterized by articular symptoms, butterfly erythema, recurrent pleurisy, pericarditis, generalized adenopathy, splenomegaly, as well as CNS involvement and progressive renal failure. The sera of most patients (over 98%) contain antinuclear antibodies, including anti-DNA antibodies. High titers of anti-DNA antibodies are essentially specific for SLE. Conventional treatment for this disease has been the administration of corticosteroids or immunosuppressants.

There are three forms of cutaneous lupus: chronic cutaneous lupus (also known as discoid lupus erythematosus or DLE), subacute cutaneous lupus, and acute cutaneous lupus. DLE is a disfiguring chronic disorder primarily affecting the skin with sharply circumscribed macules and plaques that display erythema, follicular plugging, scales, telangiectasia and atrophy. The condition is often precipitated by sun exposure, and the early lesions are erythematous, round scaling papules that are 5 to 10 mm in diameter and display follicular plugging. DLE lesions appear most commonly on the cheeks, nose, scalp, and ears, but they may also be generalized over the upper portion of the trunk, extensor surfaces of the extremities, and on the mucous membranes of the mouth. If left untreated, the central lesion atrophies and leaves a scar. Unlike SLE, antibodies against double-stranded DNA (e.g., DNA-binding test) are almost invariably absent in DLE.

Multiple Sclerosis is an autoimmune demyelinating disorder that is believed to be T lymphocyte dependent. MS generally exhibits a relapsing-remitting course or a chronic progressive course. The etiology of MS is unknown, however, viral infections, genetic predisposition, environment, and autoimmunity all appear to contribute to the disorder. Lesions in MS patients contain infiltrates of predominantly T lymphocyte mediated microglial cells and infiltrating macrophages. CD4+T lymphocytes are the predominant cell type present at these lesions. The hallmark of the MS lesion is plaque, an area of demyelination sharply demarcated from the usual white matter seen in MRI scans. Histological appearance of MS plaques varies with different stages of the disease. In active lesions, the blood-brain barrier is damaged, thereby permitting extravasation of serum proteins into extracellular spaces. Inflammatory cells can be seen in perivascular cuffs and throughout white matter. CD4+ T-cells, especially Th1, accumulate around postcapillary venules at the edge of the plaque and are also scattered in the white matter. In active lesions, up-regulation of adhesion molecules and markers of lymphocyte and monocyte activation, such as IL2-R and CD26 have also been observed. Demyelination in active lesions is not accompanied by destruction of oligodendrocytes. In contrast, during chronic phases of the disease, lesions are characterized by a loss of oligodendrocytes and hence, the presence of myelin oligodendrocyte glycoprotein (MOG) antibodies in the blood.

Diabetes can refer to either type 1 or type 2 diabetes. In some embodiments the degrader or its salt or composition as described herein is provided at an effective dose to treat a patient with type 1 diabetes. In one aspect the degrader or its salt or composition as described herein is provided at an effective dose to treat a patient with type 2 diabetes.

Type 1 diabetes is an autoimmune disease. An autoimmune disease results when the body's system for fighting infection (the immune system) turns against a part of the body. The pancreas then produces little or no insulin.

As examples, the degrader or its salt or composition as described herein is useful for treating or preventing a disorder selected from autoimmune oophoritis, endometriosis, autoimmune orchitis, Ord's thyroiditis, autoimmune enteropathy, coeliac disease, Hashimoto's encephalopathy, antiphospholipid syndrome (APLS) (Hughes syndrome), aplastic anemia, autoimmune lymphoproliferative syndrome (Canale-Smith syndrome), autoimmune neutropenia, Evans syndrome, pernicious anemia, pure red cell aplasia, thrombocytopenia, adipose dolorosa (Dercum's disease), adult onset Still's disease, ankylosing spondylitis, CREST syndrome, drug-induced lupus, eosinophilic fasciitis (Shulman's syndrome), Felty syndrome, IgG4-related disease, mixed connective tissue disease (MCTD), palindromic rheumatism (Hench-Rosenberg syndrome), Parry-Romberg syndrome, Parsonage-Turner syndrome, relapsing polychondritis (Meyenburg-Altherr-Uehlinger syndrome), retroperitoneal fibrosis, rheumatic fever, Schnitzler syndrome, fibromyalgia, neuromyotonia (Isaac's disease), paraneoplastic degeneration, autoimmune inner ear disease, Meniere's disease, interstitial cystitis, autoimmune pancreatitis, zika virus-related disorders, chikungunya virus-related disorders, subacute bacterial endocarditis (SBE), IgA nephropathy, IgA vasculitis, polymyalgia rheumatic, rheumatoid vasculitis, alopecia areata, autoimmune progesterone dermatitis, dermatitis herpetiformis, erythema nodosum, gestational pemphigoid, hidradenitis suppurativa, lichen sclerosus, linear IgA disease (LAD), morphea, myositis, pityriasis lichenoides et varioliformis acuta, vitiligo post-myocardial infarction syndrome (Dressler's syndrome), post-pericardiotomy syndrome, autoimmune retinopathy, Cogan syndrome, Graves opthalmopathy, ligneous conjunctivitis, Mooren's ulcer, opsoclonus myoclonus syndrome, optic neuritis, retinocochleocerebral vasculopathy (Susac's syndrome), sympathetic opthalmia, Tolosa-Hunt syndrome, interstitial lung disease, antisynthetase syndrome, Addison's disease, autoimmune polyendocrine syndrome (APS) type I, autoimmune polyendocrine syndrome (APS) type II, autoimmune polyendocrine syndrome (APS) type III, disseminated sclerosis (multiple sclerosis, pattern II), rapidly progressing glomerulonephritis (RPGN), juvenile rheumatoid arthritis, enthesitis-related arthritis, reactive arthritis (Reiter's syndrome), autoimmune hepatitis or lupoid hepatitis, primary biliary cirrhosis (PBS), primary sclerosing cholangitis, microscopic colitis, latent lupus (undifferentiated connective tissue disease (UCTD)), acute disseminated encephalomyelitis (ADEM), acute motor axonal neuropathy, anti-n-methyl-D-aspartate receptor encephalitis, Balo concentric sclerosis (Schilders disease), Bickerstaff's encephalitis, chronic inflammatory demyelinating polyneuropathy, idiopathic inflammatory demyelinating disease, Lambert-Eaton mysathenic syndrome, Oshtoran syndrome, pediatric autoimmune neuropsychiatric disorder associated with streptococcus (PANDAS), progressive inflammatory neuropathy, restless leg syndrome, stiff person syndrome, Sydenhem syndrome, transverse myelitis, lupus vasculitis, leukocytoclastic vasculitis, Microscopic Polyangiitis, polymyositis or ischemic-reperfusion injury of the eye.

In certain aspects, an effective amount of the degrader or its salt or composition as described herein is used to treat a medical disorder mediated by the Targeted Extracellular Protein. For example, when the Targeted Extracellular Protein is a complement protein, for example complement factor B, factor D, factor H, CIs, C3, or C5, then the medical disorder to be treated may be an inflammatory or immune condition, a disorder mediated by the complement cascade (including a dysfunctional cascade), or alternative complement pathway-related disorder, a disorder or abnormality of a cell that adversely affects the ability of the cell to engage in or respond to normal complement activity, or an undesired complement-mediated response to a medical treatment, such as surgery or other medical procedure or a pharmaceutical or biopharmaceutical drug administration, a blood transfusion, or other allogenic tissue or fluid administration.

In some aspects, the disorder treated by the degrader or its salt or composition as described herein is selected from fatty liver and conditions stemming from fatty liver, such as nonalcoholic steatohepatitis (NASH), liver inflammation, cirrhosis and liver failure.

In other embodiments, the degrader or its salt or composition as described herein is used to modulate an immune response prior to or during surgery or other medical procedure. Non-limiting examples are the use in connection with acute or chronic graft versus host disease, which is a common complication as a result of allogeneic tissue transplant, and can also occur as a result of a blood transfusion.

In certain embodiments, the present invention provides a method of treating or preventing dermatomyositis by administering to a subject in need thereof an effective amount of the degrader or its salt or composition as described herein.

In certain embodiments, the present invention provides a method of treating or preventing amyotrophic lateral sclerosis by administering to a subject in need thereof an effective amount of the degrader or its salt or composition as described herein.

In certain embodiments, the present invention provides a method of treating or preventing abdominal aortic aneurysm, hemodialysis complications, hemolytic anemia, or hemodialysis by administering to a subject in need thereof an effective amount of the degrader or its salt or composition as described herein.

In certain embodiments, a method is provided for the treatment or prevention of cytokine or inflammatory reactions in response to the administration of pharmaceutical or biotherapeutic (e.g. CAR T-cell therapy or monoclonal antibody therapy) in a host by administering an effective amount of the degrader or its salt or composition as described herein. Various types of cytokine or inflammatory reactions may occur in response to a number of factors, such as the administrations of biotherapeutics. In one aspect, the cytokine or inflammatory reaction is cytokine release syndrome. In one embodiment, the cytokine or inflammatory reaction is tumor lysis syndrome (which also leads to cytokine release). Symptoms of cytokine release syndrome range from fever, headache, and skin rashes to bronchospasm, hypotension and even cardiac arrest. Severe cytokine release syndrome is described as cytokine storm, and can be fatal.

In another embodiment, the disorder is episcleritis, idiopathic episcleritis, anterior episcleritis, or posterior episcleritis. In one embodiment, the disorder is idiopathic anterior uveitis, HLA-B27 related uveitis, herpetic keratouveitis, Posner Schlossman syndrome, Fuch's heterochromic iridocyclitis, or cytomegalovirus anterior uveitis.

In another embodiment, the present invention provides a method of treating or preventing a C3 glomurenopathy by administering to a subject in need thereof an effective amount of the degrader or its salt or composition as described herein. In one embodiment, the disorder is selected from dense deposit disease (DDD) and C3 glomerulonephritis (C3GN).

In yet another embodiment, the present invention provides a method of treating or preventing a IC-MPGN by administering to a subject in need thereof an effective amount of the degrader or its salt or composition as described herein.

In a further embodiment, the present invention provides a method of treating or preventing a paroxysmal nocturnal hemoglobinuria (PNH) by administering to a subject in need thereof an effective amount of the degrader or its salt or composition as described herein.

In another embodiment, the present invention provides a method of treating or preventing age-related macular degeneration (AMD) by administering to a subject in need thereof an effective amount of the degrader or its salt or composition as described herein.

In one embodiment, the present invention provides a method of treating or preventing rheumatoid arthritis by administering to a subject in need thereof an effective amount of the degrader or its salt or composition as described herein.

In one embodiment, the present invention provides a method of treating or preventing multiple sclerosis by administering to a subject in need thereof an effective amount of the degrader or its salt or composition as described herein.

In one embodiment, the present invention provides a method of treating or preventing myasthenia gravis by administering to a subject in need thereof an effective amount of the degrader or its salt or composition as described herein.

In one embodiment, the present invention provides a method of treating or preventing atypical hemolytic uremic syndrome (aHUS) by administering to a subject in need thereof an effective amount of the degrader or its salt or composition as described herein.

In one embodiment, the present invention provides a method of treating or preventing neuromyelitis optica (NMO) by administering to a subject in need thereof an effective amount of the degrader or its salt or composition as described herein.

In yet other embodiments, the present invention provides a method of treating or preventing a disorder as described below by administering to a subject in need thereof an effective amount of the degrader or its salt or composition as described herein, including: for example: vitritis, sarcoidosis, syphilis, tuberculosis, or Lyme disease; retinal vasculitis, Eales disease, tuberculosis, syphilis, or toxoplasmosis; neuroretinitis, viral retinitis, or acute retinal necrosis; varicella zoster virus, herpes simplex virus, cytomegalovirus, Epstein-Barr virus, lichen planus, or Dengue-associated disease (e.g., hemorraghic Dengue Fever); Masquerade syndrome, contact dermatitis, trauma induced inflammation, UVB induced inflammation, eczema, granuloma annulare, or acne.

In additional embodiments, the disorder is selected from: acute myocardial infarction, aneurysm, cardiopulmonary bypass, dilated cardiomyopathy, complement activation during cardiopulmonary bypass operations, coronary artery disease, restenosis following stent placement, or percutaneous transluminal coronary angioplasty (PTCA); antibody-mediated transplant rejection, anaphylactic shock, anaphylaxis, allogenic transplant, humoral and vascular transplant rejection, graft dysfunction, graft-versus-host disease, Graves' disease, adverse drug reactions, or chronic graft vasculopathy; allergic bronchopulmonary aspergillosis, allergic neuritis, drug allergy, radiation- induced lung injury, eosinophilic pneumonia, radiographic contrast media allergy, bronchiolitis obliterans, or interstitial pneumonia; parkinsonism-dementia complex, sporadic frontotemporal dementia, frontotemporal dementia with Parkinsonism linked to chromosome 17, frontotemporal lobar degeneration, tangle only dementia, cerebral amyloid angiopathy, cerebrovascular disorder, certain forms of frontotemporal dementia, chronic traumatic encephalopathy (CTE), PD with dementia (PDD), argyrophilic grain dementia, dementia pugilistica, dementia with Lewy Bodies (DLB), or multi-infarct dementia; Creutzfeldt-Jakob disease, Huntington's disease, multifocal motor neuropathy (MMN), prion protein cerebral amyloid angiopathy, polymyositis, postencephalitic parkinsonism, subacute sclerosing panencephalitis, non-Guamanian motor neuron disease with neurofibrillary tangles, neural regeneration, or diffuse neurofibrillary tangles with calcification.

In certain embodiments the disorder is Graves' eye disease.

In certain embodiments the disorder is Graves' ophthalmopathy.

In certain embodiments the disorder is Graves' orbitopathy.

In certain embodiments the disorder is thyroid eye disease.

In certain embodiments the disorder is neuromyelitis optica spectrum disorder.

In certain embodiments the disorder is myelin oligodendrocyte glycoprotein antibody-associated disease (MOGAD).

In further embodiments, the disorder is selected from: atopic dermatitis, dermatitis, dermatomyositis bullous pemphigoid, scleroderma, sclerodermatomyositis, psoriatic arthritis, pemphigus vulgaris, Discoid lupus erythematosus, cutaneous lupus, chilblain lupus erythematosus, or lupus erythematosus-lichen planus overlap syndrome; cryoglobulinemic vasculitis, mesenteric/enteric vascular disorder, peripheral vascular disorder, antineutrophil cytoplasm antibody (ANCA)-associated vasculitis (AAV), IL-2 induced vascular leakage syndrome, or immune complex vasculitis; angioedema, low platelets (HELLP) syndrome, sickle cell disease, platelet refractoriness, red cell casts, or typical or infectious hemolytic uremic syndrome (tHUS); hematuria, hemorrhagic shock, drug-induced thrombocytopenia, autoimmune hemolytic anemia (AIHA), azotemia, blood vessel and/or lymph vessel inflammation, rotational atherectomy, or delayed hemolytic transfusion reaction; British type amyloid angiopathy, Buerger's disease, bullous pemphigoid, C1q nephropathy, cancer, or catastrophic antiphospholipid syndrome.

In other embodiments, the disorder is selected from: wet (exudative) AMD, dry (non-exudative) AMD, chorioretinal degeneration, choroidal neovascularization (CNV), choroiditis, loss of RPE function, loss of vision (including loss of visual acuity or visual field), loss of vision from AMD, retinal damage in response to light exposure, retinal degeneration, retinal detachment, retinal dysfunction, retinal neovascularization (RNV), retinopathy of prematurity, pathological myopia, or RPE degeneration; pseudophakic bullous keratopathy, symptomatic macular degeneration related disorder, optic nerve degeneration, photoreceptor degeneration, cone degeneration, loss of photoreceptor cells, pars planitis, scleritis, proliferative vitreoretinopathy, or formation of ocular drusen; chronic urticaria, Churg-Strauss syndrome, cold agglutinin disease (CAD), corticobasal degeneration (CBD), cryoglobulinemia, cyclitis, damage of the Bruch's membrane, Degos disease, diabetic angiopathy, elevated liver enzymes, endotoxemia, epidermolysis bullosa, or epidermolysis bullosa acquisita; essential mixed cryoglobulinemia, excessive blood urea nitrogen-BUN, focal segmental glomerulosclerosis, Gerstmann-Straussler-Scheinker disease, giant cell arteritis, gout, Hallervorden-Spatz disease, Hashimoto's thyroiditis, Henoch-Schonlein purpura nephritis, or abnormal urinary sediments; hepatitis, hepatitis A, hepatitis B, hepatitis C or human immunodeficiency virus (HIV), a viral infection more generally, for example selected from Flaviviridae, Retroviruses, Coronaviridae, Poxviridae, Adenoviridae, Herpesviridae, Caliciviridae, Reoviridae, Picornaviridae, Togaviridae, Orthomyxoviridae, Rhabdoviridae, or Hepadnaviridae; Neisseria meningitidis, shiga toxin E. coli-related hemolytic uremic syndrome (STEC-HUS), hemolytic uremic syndrome (HUS); Streptococcus, or poststreptococcal glomerulonephritis.

In further embodiments, the disorder is selected from: hyperlipidemia, hypertension, hypoalbuminemia, hypobolemic shock, hypocomplementemic urticarial vasculitis syndrome, hypophosphastasis, hypovolemic shock, idiopathic pneumonia syndrome, or idiopathic pulmonary fibrosis; inclusion body myositis, intestinal ischemia, iridocyclitis, iritis, juvenile chronic arthritis, Kawasaki's disease (arteritis), or lipiduria; membranoproliferative glomerulonephritis (MPGN) I, microscopic polyangiitis, mixed cryoglobulinemia, molybdenum cofactor deficiency (MoCD) type A, pancreatitis, panniculitis, Pick's disease, polyarteritis nodosa (PAN), progressive subcortical gliosis, proteinuria, reduced glomerular filtration rate (GFR), or renovascular disorder; multiple organ failure, multiple system atrophy (MSA), myotonic dystrophy, Niemann-Pick disease type C, chronic demyelinating diseases, or progressive supranuclear palsy; spinal cord injury, spinal muscular atrophy, spondyloarthropathies, Reiter's syndrome, spontaneous fetal loss, recurrent fetal loss, pre-eclampsia, synucleinopathy, Takayasu's arteritis, post-partum thryoiditis, thyroiditis, Type I cryoglobulinemia, Type II mixed cryoglobulinemia, Type III mixed cryoglobulinemia, ulcerative colitis, uremia, urticaria, venous gas embolus (VGE), or Wegener's granulomatosis; von Hippel-Lindau disease, histoplasmosis of the eye, hard drusen, soft drusen, pigment clumping, or photoreceptor and/or retinal pigmented epithelia (RPE) loss,.

Examples of eye disorders that may be treated according to the compositions and methods disclosed herein include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, posterior uveitis (including toxoplasmosis), pan-uveitis, an inflammatory disease of the vitreous or retina, endophthalmitis prophylaxis and treatment, macular edema, macular degeneration, age related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof.

In other embodiments, the disorder is selected from glaucoma, diabetic retinopathy, blistering cutaneous diseases (including bullous pemphigoid, pemphigus, and epidermolysis bullosa), ocular cicatrical pemphigoid, uveitis, adult macular degeneration, diabetic retinopa retinitis pigmentosa, macular edema, diabetic macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, postoperative inflammation, and retinal vein occlusion, or central retinal vein occulusion (CVRO).

Disorders that may be treated or prevented by the degrader or its salt or composition as described herein also include, but are not limited to: hereditary angioedema, capillary leak syndrome, hemolytic uremic syndrome (HUS), neurological disorders, Guillain Barre Syndrome, diseases of the central nervous system and other neurodegenerative conditions, glomerulonephritis (including membrane proliferative glomerulonephritis), SLE nephritis, proliferative nephritis, liver fibrosis, tissue regeneration and neural regeneration, or Barraquer-Simons Syndrome; inflammatory effects of sepsis, systemic inflammatory response syndrome (SIRS), disorders of inappropriate or undesirable complement activation, interleukin-2 induced toxicity during IL-2 therapy, inflammatory disorders, inflammation of autoimmune diseases, system lupus erythematosus (SLE), lupus nephritides, arthritis, immune complex disorders and autoimmune diseases, systemic lupus, or lupus erythematosus; ischemia/reperfusion injury (I/R injury), myocardial infarction, myocarditis, post-ischemic reperfusion conditions, balloon angioplasty, atherosclerosis, post-pump syndrome in cardiopulmonary bypass or renal bypass, renal ischemia, mesenteric artery reperfusion after aortic reconstruction, antiphospholipid syndrome, autoimmune heart disease, ischemia-reperfusion injuries, obesity, or diabetes; Alzheimer's dementia, stroke, schizophrenia, traumatic brain injury, trauma, Parkinson's disease, epilepsy, transplant rejection, prevention of fetal loss, biomaterial reactions (e.g. in hemodialysis, implants), hyperacute allograft rejection, xenograft rejection, transplantation, psoriasis, burn injury, thermal injury including burns or frostbite, or crush injury; asthma, allergy, acute respiratory distress syndrome (ARDS), cystic fibrosis, adult respiratory distress syndrome, dyspnea, hemoptysis, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), bronchoconstriction, hypersensitivity pneumonitis, parasitic diseases, Goodpasture's Syndrome (anti-glomerular basement membrane nephritis), pulmonary vasculitis, Pauci-immune vasculitis, or immune complex- associated inflammation.

In another embodiment, a method for the treatment of sickle cell in a host is provided that includes the administration of an effective amount of the degrader or its salt or composition as described herein. In one embodiment, a method for the treatment of immunothrombocytopenic purpura (ITP), thrombotic thrombocytopenic purpura (TTP), or idiopathic thrombocytopenic purpura (ITP) in a host is provided that includes the administration of an effective amount of the degrader or its salt or composition as described herein. In one embodiment, a method for the treatment of ANCA-vasculitis in a host is provided that includes the administration of an effective amount of the degrader or its salt or composition as described herein. In one embodiment, a method for the treatment of IgA nephropathy in a host is provided that includes the administration of an effective amount of the degrader or its salt or composition as described herein. In one embodiment, a method for the treatment of rapidly progressing glomerulonephritis (RPGN), in a host is provided that includes the administration of an effective amount of the degrader or its salt or composition as described herein. In one embodiment, a method for the treatment of lupus nephritis, in a host is provided that includes the administration of an effective amount of the degrader or its salt or composition as described herein. In one embodiment, a method for the treatment of hemorraghic dengue fever, in a host is provided that includes the administration of an effective amount of the degrader or its salt or composition as described herein.

In another aspect, an effective amount of the degrader or its salt or composition as described herein is used to treat an abnormal proliferation disorder such as a tumor or cancer.

Non-limiting examples of cancers that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AIL) (e.g., B-cell AIL, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In another embodiment, the disorder is myelodysplastic syndrome (MDS).

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein.

Additional examples of solid tumors include, but are not limited to, squamous cell carcinoma, colon cancer, breast cancer, prostate cancer, lung cancer, liver cancer, pancreatic cancer, and melanoma.

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

In certain embodiments, the condition is associated with an immune response.

Cutaneous contact hypersensitivity and asthma are just two examples of immune responses that can be associated with significant morbidity. Others include atopic dermatitis, eczema, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis. Immunologically mediated leukocyte infiltration also occurs at sites other than the skin, such as in the airways in asthma and in the tear producing gland of the eye in keratoconjunctivitis sicca.

In other non-limiting embodiments, degraders of the present invention are used as topical agents in treating contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions. The novel method may also be useful in reducing the infiltration of skin by malignant leukocytes in diseases such as mycosis fungoides. These compounds can also be used to treat an aqueous-deficient dry eye state (such as immune mediated keratoconjunctivitis) in a patient suffering therefrom, by administering the compound topically to the eye.

Exemplary cancers which may be treated by the present disclosed compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis;; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+ DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

Nonlimiting general examples of disorders mediated by extracellular proteins also include, but are not limited to: AMD, macular edema, DME, diabetic retinopathy, mCNV; neurodegenerative disorders, metastatic colorectal cancer, non-squamous non-small-cell lung carcinoma, GMB, metastatic renal cell carcinoma, cervical cancer, AA amyloidosis, amyloid light chain (AL) amyloidosis, ankylosing spondylitis, antiphospholipid Ab syndrome, asthma, progression of parasite Schistosoma mansoni infection (IL-13), ATTR amyloidosis, Behcet syndrome, sepsis, inflammation, rheumatoid arthritis, atherosclerosis, ischemia/reperfusion injury; MGUS, Necrobiotic xanthogranuloma, JIA, psoriatic arthritis, plaque psoriasis, Crohn's disease, ulcerative colitis, Hidradenitis suppurativa uveitis; GvH disease; Castleman's disease, liver fibrosis, Still's Disease; cutaneous skin diseases including atopic dermatitis, transplant rejection, multiple myeloma, osteosclerotic multiple myeloma with peripheral neuropathy; pancreatic tumors; paraproteinemia (NR), prostate, gastric cancer; glioblastoma multiforme; acute coronary syndrome; hyperlipidemia (Rare/Broad), chronic urticaria, scleroderma, scleromyxedema, hereditary angioedema, clotting disorders, heparin-induced thrombocytopenia; Acquired Von Willebrand disease (AVWD), antiphospholipid antibody syndrome (APS or APLS); cryoglobulinemia; granulomatosis with polyangiitis (Wegener's)-sub-type of ANCA-associated vasculitis; idiopathic (Immune); thrombocytopenic purpura; IgG4-RD; Non-IgM MGUS; X-linked hypophosphatemia; Multiple System Atrophy (MSA), Parkinson's disease, Cachexia, Sarcopenia, Sporadic inclusion body myositis, muscular dystrophy, COPD; rhabdomyolysis; dialysis-related amyloidosis; focal segmental glomerulosclerosis (FSGS); IgA nephropathy (IgAN) and Henoch Schonlein Purpura (HSP); acute disseminated encephalomyelitis (ADEM); acute inflammatory demyelinating polyneuropathy (AIDP); Guillaine-Barre Syndrome; Alzheimer' disease & FTD; chronic inflammatory demyelinating polyneuropathy (CIDP); Creutzfeldt-Jakob disease (CJD); Huntington's disease; Miller Fisher Syndrome; Neuromyelitis optica spectrum disorder (NMOSD); Opsoclonus-myoclonus syndrome; PANDAS syndrome (pediatric autoimmune neuropsychiatric disorders associated with Streptcoccal infections); Transverse myelitis; Emphysema, respiratory failure; Anthrax; Botulism; Sepsis; Staph. aureus toxic shock syndrome; Tetanus; Transplantation; Acromegaly; Cushing's disease; prion disease; secondary membranous nephropathy; and vasculitis.

### IX. Representative Compounds of the Present Invention

| Ligand # | Structure |
|---|---|
| 1 | 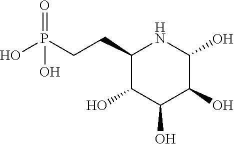 [SMILES: O=P(O)(O)CC[C@H]1N[C@H](O)[C@@H](O)[C@@H](O)[C@@H]1O] |
| 2 | 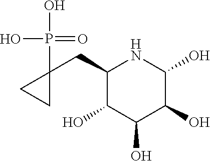 [SMILES: O=P(O)(O)C1(C[C@H]2N[C@H](O)[C@@H](O)[C@@H](O)[C@@H]2O)CC1] |
| 3 | 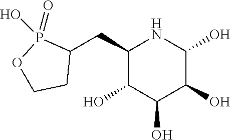 [SMILES: O=P1(O)OCCC1C[C@H]1N[C@H](O)[C@@H](O)[C@@H](O)[C@@H]1O] |
| 4 | 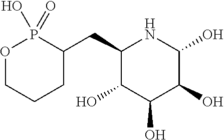 [SMILES: O=P1(O)OCCCC1C[C@H]1N[C@H](O)[C@@H](O)[C@@H](O)[C@@H]1O] |
| 5 | 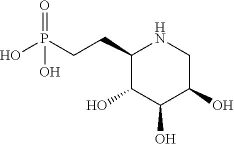 [SMILES: O=P(O)(O)CC[C@H]1NC[C@@H](O)[C@@H](O)[C@@H]1O] |
| 6 | 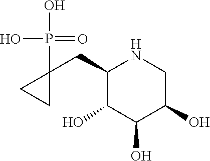 [SMILES: O=P(O)(O)C1(C[C@H]2NC[C@@H](O)[C@@H](O)[C@@H]2O)CC1] |
| 7 | 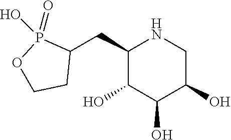 [SMILES: O=P1(O)OCCC1C[C@H]1NC[C@@H](O)[C@@H](O)[C@@H]1O] |
| 8 | 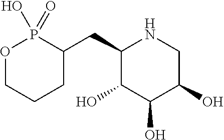 [SMILES: O=P1(O)OCCCC1C[C@H]1NC[C@@H](O)[C@@H](O)[C@@H]1O] |
| 9 | 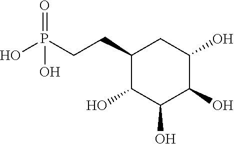 [SMILES: O=P(O)(O)CC[C@H]1C[C@H](O)[C@@H](O)[C@@H](O)[C@@H]1O] |
| 10 | 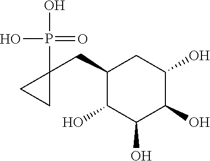 [SMILES: O=P(O)(O)C1(C[C@H]2C[C@H](O)[C@@H](O)[C@@H](O)[C@@H]2O)CC1] |
| 11 | 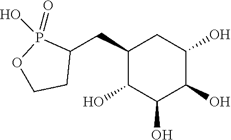 [SMILES: O=P1(O)OCCC1C[C@H]1C[C@H](O)[C@@H](O)[C@@H](O)[C@@H]1O] |
| 12 | 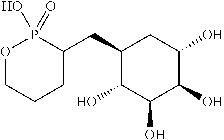 [SMILES: O=P1(O)OCCCC1C[C@H]1C[C@H](O)[C@@H](O)[C@@H](O)[C@@H]1O] |
| 13 | 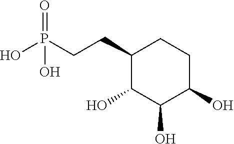 [SMILES: O=P(O)(O)CC[C@H]1CC[C@@H](O)[C@@H](O)[C@@H]1O] |
| 14 | 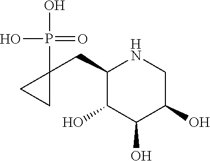 [SMILES: O=P(O)(O)C1(C[C@H]2NC[C@@H](O)[C@@H](O)[C@@H]2O)CC1] |
| 15 | 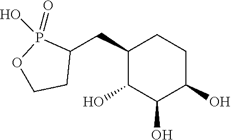 [SMILES: O=P1(O)OCCC1C[C@H]1CC[C@@H](O)[C@@H](O)[C@@H]1O] |
| 16 | 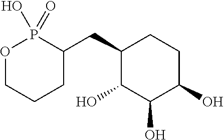 [SMILES: O=P1(O)OCCCC1C[C@H]1CC[C@@H](O)[C@@H](O)[C@@H]1O] |
| 17 | 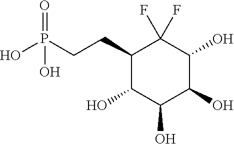 [SMILES: O=P(O)(O)CC[C@@H]1[C@@H](O)[C@H](O)[C@H](O)[C@@H](O)C1(F)F] |
| 18 | 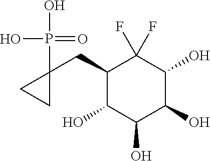 [SMILES: O=P(O)(O)C1(C[C@@H]2[C@@H](O)[C@H](O)[C@H](O)[C@@H](O)C2(F)F)CC1] |
| 19 | 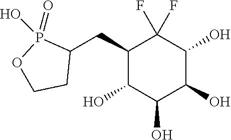 [SMILES: O=P1(O)OCCC1C[C@@H]1[C@@H](O)[C@H](O)[C@H](O)[C@@H](O)C1(F)F] |
| 20 | 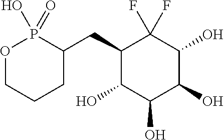 [SMILES: O=P1(O)OCCCC1C[C@@H]1[C@@H](O)[C@H](O)[C@H](O)[C@@H](O)C1(F)F] |
| 21 | 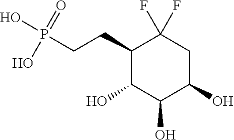 [SMILES: O=P(O)(O)CC[C@@H]1[C@@H](O)[C@H](O)[C@H](O)CC1(F)F] |
| 22 | 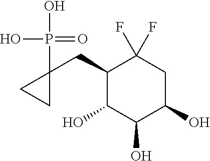 [SMILES: O=P(O)(O)C1(C[C@@H]2[C@@H](O)[C@H](O)[C@H](O)CC2(F)F)CC1] |
| 23 | 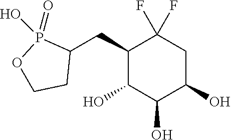 [SMILES: O=P1(O)OCCC1C[C@@H]1[C@@H](O)[C@H](O)[C@H](O)CC1(F)F] |
| 24 | 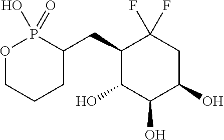 [SMILES: O=P1(O)OCCCC1C[C@@H]1[C@@H](O)[C@H](O)[C@H](O)CC1(F)F] |
| 25 | 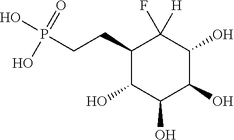 [SMILES: O=P(O)(O)CC[C@H]1C(F)[C@H](O)[C@@H](O)[C@@H](O)[C@@H]1O] |
| 26 | 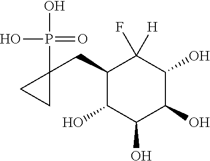 [SMILES: O=P(O)(O)C1(C[C@H]2C(F)[C@H](O)[C@@H](O)[C@@H](O)[C@@H]2O)CC1] |
| 27 | 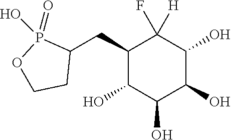 [SMILES: O=P1(O)OCCC1C[C@H]1C(F)[C@H](O)[C@@H](O)[C@@H](O)[C@@H]1O] |
| 28 | 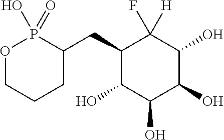 [SMILES: O=P1(O)OCCCC1C[C@H]1C(F)[C@H](O)[C@@H](O)[C@@H](O)[C@@H]1O] |
| 29 | 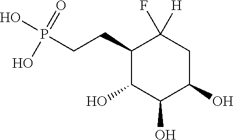 [SMILES: O=P(O)(O)CC[C@H]1C(F)C[C@@H](O)[C@@H](O)[C@@H]1O] |
| 30 | 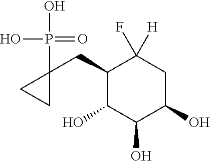 [SMILES: O=P(O)(O)C1(C[C@H]2C(F)C[C@@H](O)[C@@H](O)[C@@H]2O)CC1] |
| 31 | 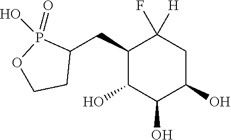 [SMILES: O=P1(O)OCCC1C[C@H]1C(F)C[C@@H](O)[C@@H](O)[C@@H]1O] |
| 32 | 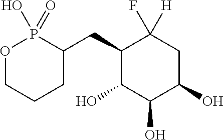 [SMILES: O=P1(O)OCCCC1C[C@H]1C(F)C[C@@H](O)[C@@H](O)[C@@H]1O] |
| 33 | 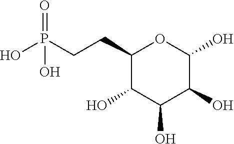 [SMILES: O=P(O)(O)CC[C@H]1O[C@H](O)[C@@H](O)[C@@H](O)[C@@H]1O] |
| 34 | 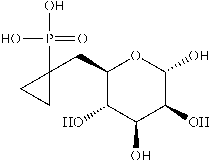 [SMILES: O=P(O)(O)C1(C[C@H]2O[C@H](O)[C@@H](O)[C@@H](O)[C@@H]2O)CC1] |
| 35 | 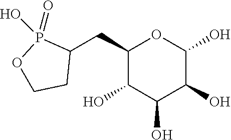 [SMILES: O=P1(O)OCCC1C[C@H]1O[C@H](O)[C@@H](O)[C@@H](O)[C@@H]1O] |
| 36 | 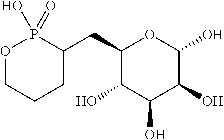 [SMILES: O=P1(O)OCCCC1C[C@H]1O[C@H](O)[C@@H](O)[C@@H](O)[C@@H]1O] |
| 38 | 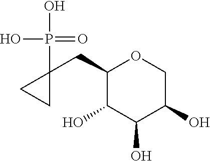 [SMILES: O=P(O)(O)C1(C[C@H]2OC[C@@H](O)[C@@H](O)[C@@H]2O)CC1] |
| 39 | 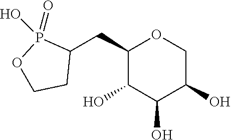 [SMILES: O=P1(O)OCCC1C[C@H]1OC[C@@H](O)[C@@H](O)[C@@H]1O] |
| 40 | 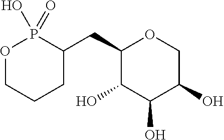 [SMILES: O=P1(O)OCCCC1C[C@H]1OC[C@@H](O)[C@@H](O)[C@@H]1O] |
| 41 | 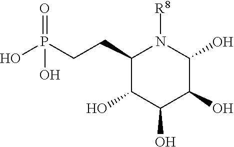 |
| 42 | 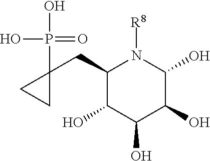 |
| 43 | 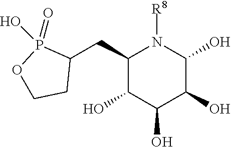 |
| 44 | 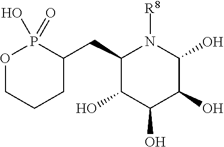 |
| 45 | 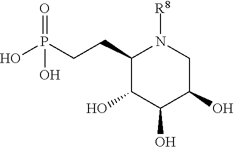 |
| 46 | 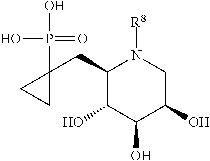 |
| 47 | 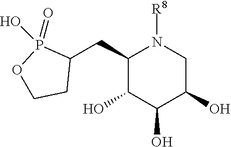 |
| 48 | 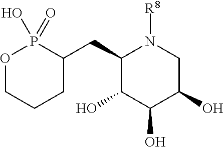 |
| 49 | 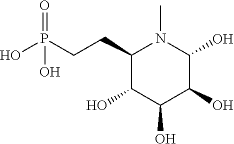 [SMILES: CN1[C@H](O)[C@@H](O)[C@@H](O)[C@H](O)[C@H]1CCP(=O)(O)O] |
| 50 | 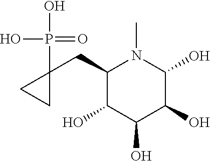 [SMILES: CN1[C@H](O)[C@@H](O)[C@@H](O)[C@H](O)[C@H]1CC1(P(=O)(O)O)CC1] |
| 51 | 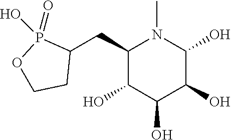 [SMILES: CN1[C@H](O)[C@@H](O)[C@@H](O)[C@H](O)[C@H]1CC1CCOP1(=O)O] |
| 52 | 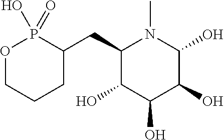 [SMILES: CN1[C@H](O)[C@@H](O)[C@@H](O)[C@H](O)[C@H]1CC1CCCOP1(=O)O] |
| 53 | 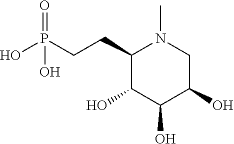 [SMILES: CN1C[C@@H](O)[C@@H](O)[C@H](O)[C@H]1CCP(=O)(O)O] |
| 54 | 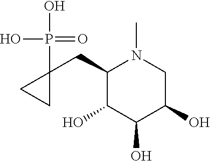 [SMILES: CN1C[C@@H](O)[C@@H](O)[C@H](O)[C@H]1CC1(P(=O)(O)O)CC1] |
| 55 | 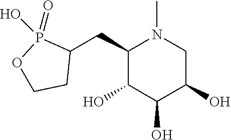 [SMILES: CN1C[C@@H](O)[C@@H](O)[C@H](O)[C@H]1CC1CCOP1(=O)O] |
| 56 | 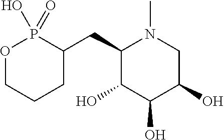 [SMILES: CN1C[C@@H](O)[C@@H](O)[C@H](O)[C@H]1CC1CCCOP1(=O)O] |
| 57 | 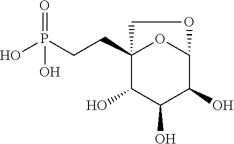 [SMILES: O=P(O)(O)CC[C@@]12CO[C@@H](O1)[C@@H](O)[C@@H](O)[C@@H]2O] |
| 58 | 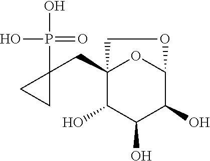 [SMILES: O=P(O)(O)C1(C[C@@]23CO[C@@H](O2)[C@@H](O)[C@@H](O)[C@@H]3O)CC1] |
| 59 | 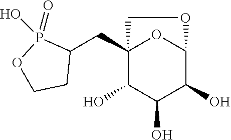 [SMILES: O=P1(O)OCCC1C[C@@]12CO[C@@H](O1)[C@@H](O)[C@@H](O)[C@@H]2O] |
| 60 | 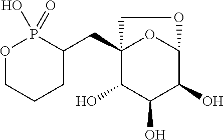 [SMILES: O=P1(O)OCCCC1C[C@@]12CO[C@@H](O1)[C@@H](O)[C@@H](O)[C@@H]2O] |
| 61 | 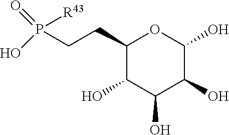 |
| 62 | 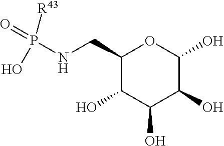 |
| 64 | 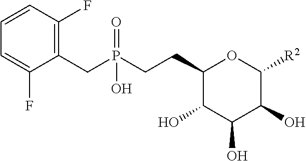 |
| 65 | 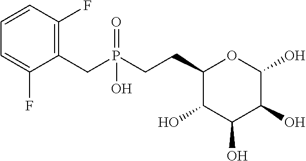 [SMILES: O=P(O)(CC[C@H]1O[C@H](O)[C@@H](O)[C@@H](O)[C@@H]1O)Cc1c(F)cccc1F] |
| 66 | 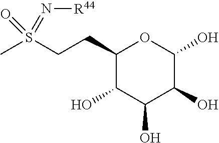 |
| 67 | 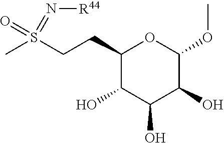 |
| 68 | 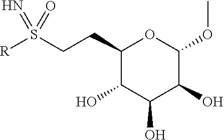 |
| 69 | 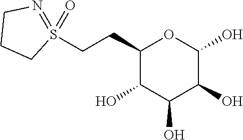 [SMILES: O=S1(CC[C@H]2O[C@H](O)[C@@H](O)[C@@H](O)[C@@H]2O)=NCCC1] |
| 70 | 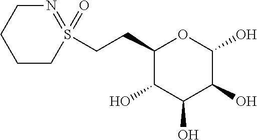 [SMILES: O=S1(CC[C@H]2O[C@H](O)[C@@H](O)[C@@H](O)[C@@H]2O)=NCCCC1] |
| 71 | 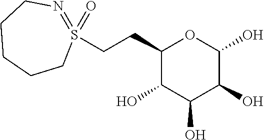 [SMILES: O=S1(CC[C@H]2O[C@H](O)[C@@H](O)[C@@H](O)[C@@H]2O)=NCCCCC1] |
| 72 | 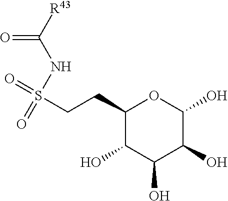 |
| 73 | 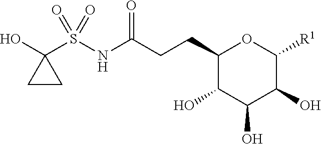 |
| 74 | 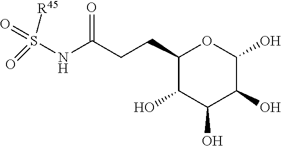 |
| 75 | 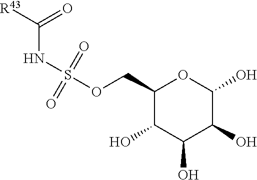 |
| 76 | 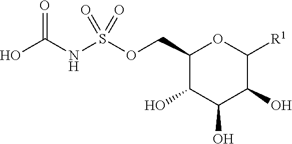 |
| 77 | 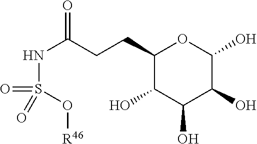 |
| 78 | 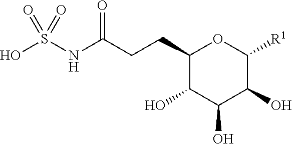 |
| 79 | 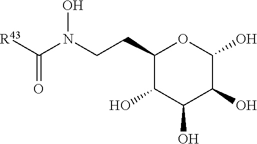 |
| 80 | 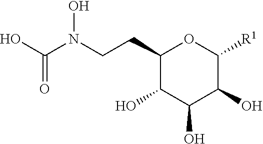 |
| 81 | 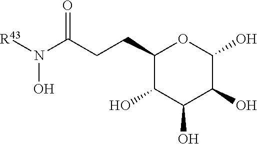 |
| 82 | 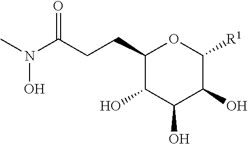 |
| 83 | 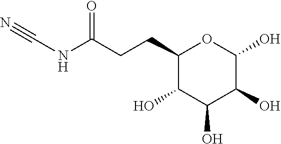 [SMILES: N#CNC(=O)CC[C@H]1O[C@H](O)[C@@H](O)[C@@H](O)[C@@H]1O] |
| 84 | 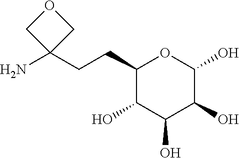 [SMILES: NC1(CC[C@H]2O[C@H](O)[C@@H](O)[C@@H](O)[C@@H]2O)COC1] |
| 85 | 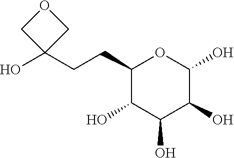 [SMILES: O[C@@H]1[C@H](O)[C@@H](O)O[C@H](CCC2(O)COC2)[C@H]1O] |
| 86 | 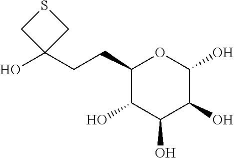 [SMILES: O[C@@H]1[C@H](O)[C@@H](O)O[C@H](CCC2(O)CSC2)[C@H]1O] |
| 87 | 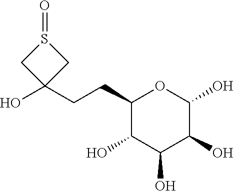 [SMILES: O=S1CC(O)(CC[C@H]2O[C@H](O)[C@@H](O)[C@@H](O)[C@@H]2O)C1] |
| 88 | 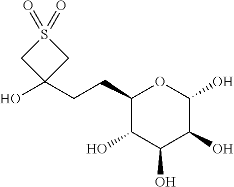 [SMILES: O=S1(=O)CC(O)(CC[C@H]2O[C@H](O)[C@@H](O)[C@@H](O)[C@@H]2O)C1] |
| 89 | 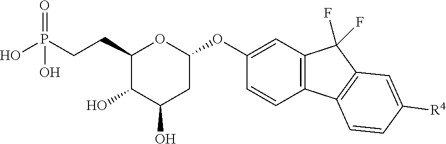 |
| 90 | 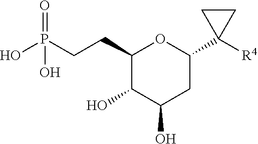 |
| 91 | 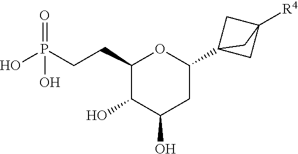 |
| 92 | 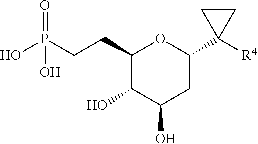 |
| 93 | 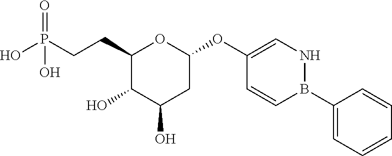 [SMILES: O=P(O)(O)CC[C@H]1O[C@H](OC2=CNB(c3ccccc3)C=C2)C[C@@H](O)[C@@H]1O] |
| 94 | 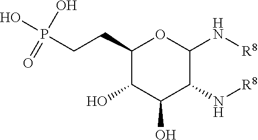 |
| 95 | 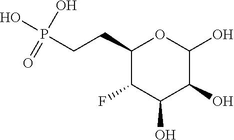 [SMILES: O=P(O)(O)CC[C@H]1OC(O)[C@@H](O)[C@@H](O)[C@@H]1F] |
| 96 | 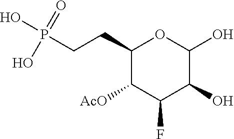 [SMILES: CC(=O)O[C@H]1[C@H](F)[C@H](O)C(O)O[C@@H]1CCP(=O)(O)O] |
| 97 | 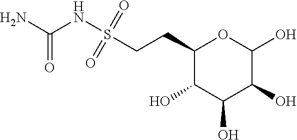 [SMILES: NC(=O)NS(=O)(=O)CC[C@H]1OC(O)[C@@H](O)[C@@H](O)[C@@H]1O] |
| 98 | 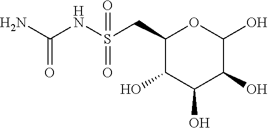 [SMILES: NC(=O)NS(=O)(=O)C[C@H]1OC(O)[C@@H](O)[C@@H](O)[C@@H]1O] |
| 99 | 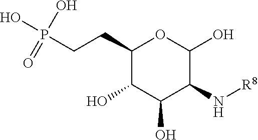 |
| 100 | 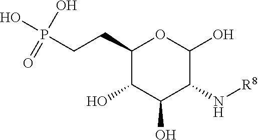 |
| 101 | 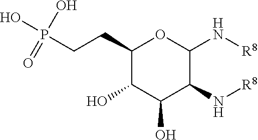 |
| 102 | 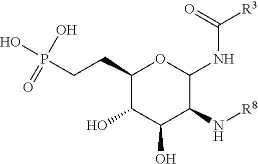 |
| 103 | 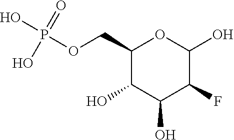 [SMILES: O=P(O)(O)OC[C@H]1OC(O)[C@@H](F)[C@@H](O)[C@@H]1O] |
| 104 | 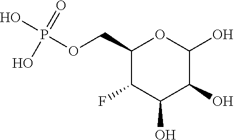 [SMILES: O=P(O)(O)OC[C@H]1OC(O)[C@@H](O)[C@@H](O)[C@@H]1F] |
| 105 | 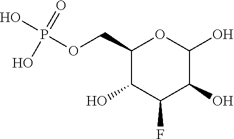 [SMILES: O=P(O)(O)OC[C@H]1OC(O)[C@@H](O)[C@@H](F)[C@@H]1O] |
| 106 | 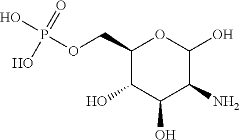 [SMILES: N[C@@H]1C(O)O[C@H](COP(=O)(O)O)[C@@H](O)[C@@H]1O] |
| 107 | 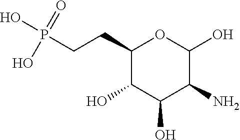 [SMILES: N[C@@H]1C(O)O[C@H](CCP(=O)(O)O)[C@@H](O)[C@@H]1O] |
| 108 | 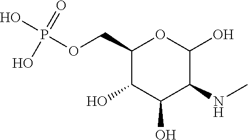 [SMILES: CN[C@@H]1C(O)O[C@H](COP(=O)(O)O)[C@@H](O)[C@@H]1O] |
| 109 | 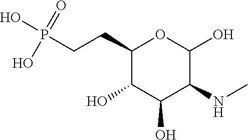 [SMILES: CN[C@@H]1C(O)O[C@H](CCP(=O)(O)O)[C@@H](O)[C@@H]1O] |
| 110 | 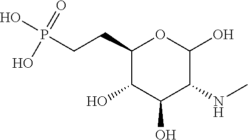 [SMILES: CN[C@H]1C(O)O[C@H](CCP(=O)(O)O)[C@@H](O)[C@@H]1O] |
| 111 | 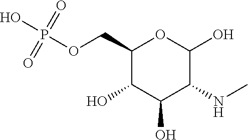 |
| 112 | 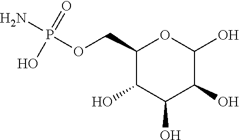 [SMILES: NP(=O)(O)OC[C@H]1OC(O)[C@@H](O)[C@@H](O)[C@@H]1O] |
| 113 | 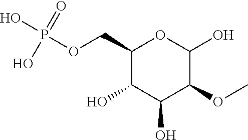 [SMILES: CO[C@@H]1C(O)O[C@H](COP(=O)(O)O)[C@@H](O)[C@@H]1O] |
| 114 | 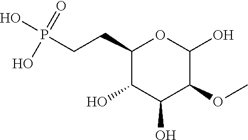 [SMILES: CO[C@@H]1C(O)O[C@H](CCP(=O)(O)O)[C@@H](O)[C@@H]1O] |
| 115 | 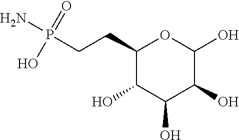 [SMILES: NP(=O)(O)CC[C@H]1OC(O)[C@@H](O)[C@@H](O)[C@@H]1O] |
| 116 | 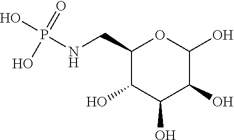 [SMILES: O=P(O)(O)NC[C@H]1OC(O)[C@@H](O)[C@@H](O)[C@@H]1O] |
| 117 | 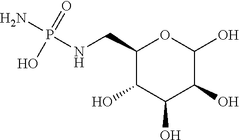 [SMILES: NP(=O)(O)NC[C@H]1OC(O)[C@@H](O)[C@@H](O)[C@@H]1O] |
| 118 | 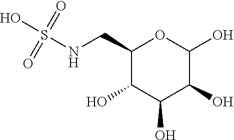 [SMILES: O=S(=O)(O)NC[C@H]1OC(O)[C@@H](O)[C@@H](O)[C@@H]1O] |
| 119 | 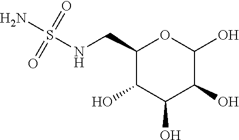 [SMILES: NS(=O)(=O)NC[C@H]1OC(O)[C@@H](O)[C@@H](O)[C@@H]1O] |
| 120 | 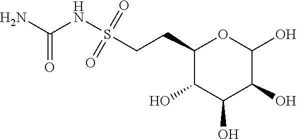 [SMILES: NC(=O)NS(=O)(=O)CC[C@H]1OC(O)[C@@H](O)[C@@H](O)[C@@H]1O] |
| 121 | 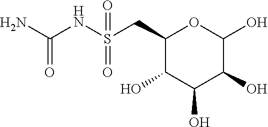 [SMILES: NC(=O)NS(=O)(=O)C[C@H]1OC(O)[C@@H](O)[C@@H](O)[C@@H]1O] |
| 122 | 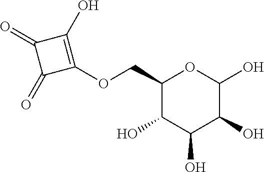 [SMILES: O=c1c(O)c(OC[C@H]2OC(O)[C@@H](O)[C@@H](O)[C@@H]2O)c1=O] |
| 123 | 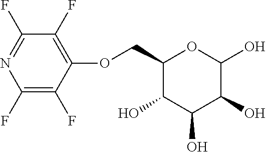 [SMILES: OC1O[C@H](COc2c(F)c(F)nc(F)c2F)[C@@H](O)[C@H](O)[C@@H]1O] |
| 124 | 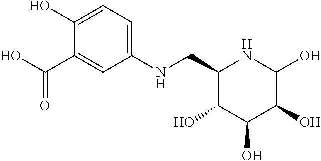 [SMILES: O=C(O)c1cc(NC[C@H]2NC(O)[C@@H](O)[C@@H](O)[C@@H]2O)ccc1O] |
| 125 | 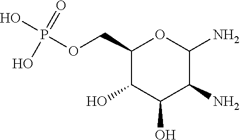 [SMILES: NC1O[C@H](COP(=O)(O)O)[C@@H](O)[C@H](O)[C@@H]1N] |
| 126 | 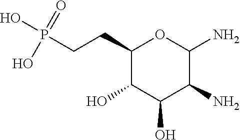 [SMILES: NC1O[C@H](CCP(=O)(O)O)[C@@H](O)[C@H](O)[C@@H]1N] |
| 127 | 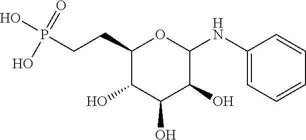 [SMILES: O=P(O)(O)CC[C@H]1OC(Nc2ccccc2)[C@@H](O)[C@@H](O)[C@@H]1O] |
| 128 | 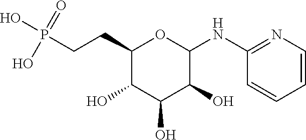 [SMILES: O=P(O)(O)CC[C@H]1OC(Nc2ccccn2)[C@@H](O)[C@@H](O)[C@@H]1O] |
| 129 | 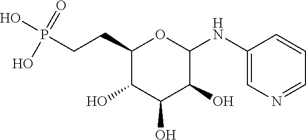 [SMILES: O=P(O)(O)CC[C@H]1OC(Nc2cccnc2)[C@@H](O)[C@@H](O)[C@@H]1O] |
| 130 | 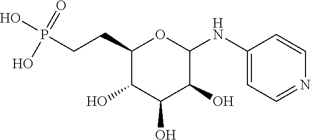 [SMILES: O=P(O)(O)CC[C@H]1OC(Nc2ccncc2)[C@@H](O)[C@@H](O)[C@@H]1O] |
| 131 | 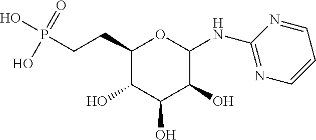 [SMILES: O=P(O)(O)CC[C@H]1OC(Nc2ncccn2)[C@@H](O)[C@@H](O)[C@@H]1O] |
| 132 | 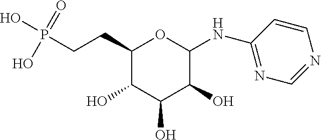 [SMILES: O=P(O)(O)CC[C@H]1OC(Nc2ccncn2)[C@@H](O)[C@@H](O)[C@@H]1O] |
| 133 | 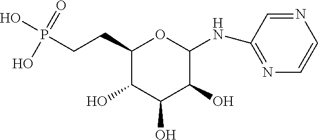 [SMILES: O=P(O)(O)CC[C@H]1OC(Nc2cnccn2)[C@@H](O)[C@@H](O)[C@@H]1O] |
| 134 | 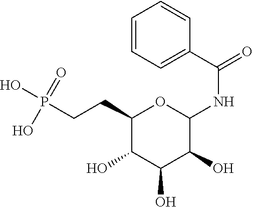 [SMILES: O=C(NC1O[C@H](CCP(=O)(O)O)[C@@H](O)[C@H](O)[C@@H]1O)c1ccccc1] |
| 135 | 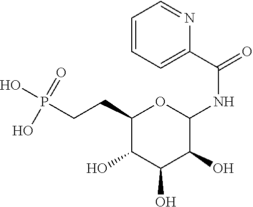 [SMILES: O=C(NC1O[C@H](CCP(=O)(O)O)[C@@H](O)[C@H](O)[C@@H]1O)c1ccccn1] |
| 136 | 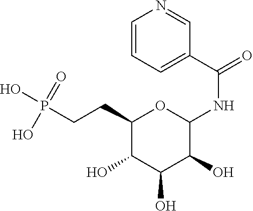 [SMILES: O=C(NC1O[C@H](CCP(=O)(O)O)[C@@H](O)[C@H](O)[C@@H]1O)c1cccnc1] |
| 137 | 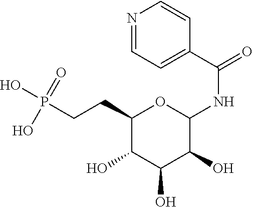 [SMILES: O=C(NC1O[C@H](CCP(=O)(O)O)[C@@H](O)[C@H](O)[C@@H]1O)c1ccncc1] |
| 138 | 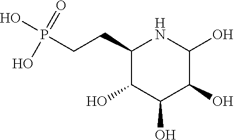 [SMILES: O=P(O)(O)CC[C@H]1NC(O)[C@@H](O)[C@@H](O)[C@@H]1O] |
| 139 | 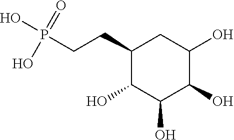 [SMILES: O=P(O)(O)CC[C@H]1CC(O)[C@@H](O)[C@@H](O)[C@@H]1O] |
| 140 | 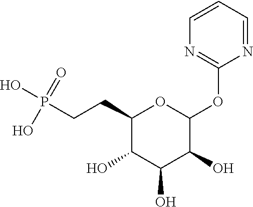 [SMILES: O=P(O)(O)CC[C@H]1OC(Oc2ncccn2)[C@@H](O)[C@@H](O)[C@@H]1O] |
| 141 | 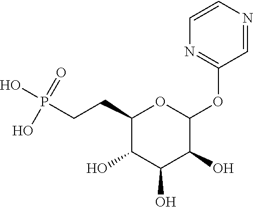 [SMILES: O=P(O)(O)CC[C@H]1OC(Oc2cnccn2)[C@@H](O)[C@@H](O)[C@@H]1O] |
| 142 | 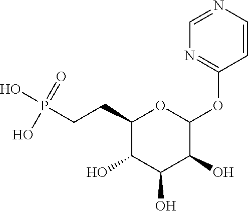 [SMILES: O=P(O)(O)CC[C@H]1OC(Oc2ccncn2)[C@@H](O)[C@@H](O)[C@@H]1O] |
| 143 | 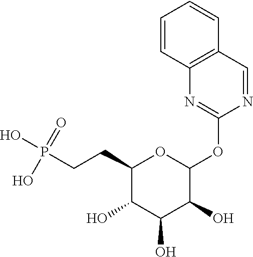 [SMILES: O=P(O)(O)CC[C@H]1OC(Oc2ncc3ccccc3n2)[C@@H](O)[C@@H](O)[C@@H]1O] |
| 144 | 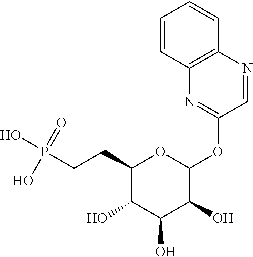 [SMILES: O=P(O)(O)CC[C@H]1OC(Oc2cnc3ccccc3n2)[C@@H](O)[C@@H](O)[C@@H]1O] |
| 145 | 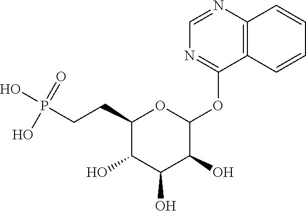 [SMILES: O=P(O)(O)CC[C@H]1OC(Oc2ncnc3ccccc23)[C@@H](O)[C@@H](O)[C@@H]1O] |
| 146 | 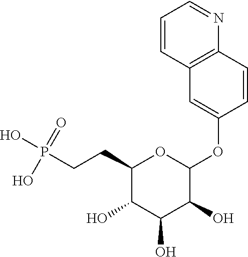 [SMILES: O=P(O)(O)CC[C@H]1OC(Oc2ccc3ncccc3c2)[C@@H](O)[C@@H](O)[C@@H]1O] |
| 147 | 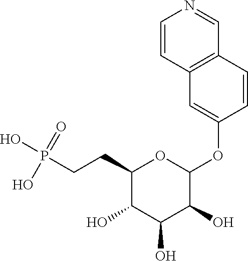 [SMILES: O=P(O)(O)CC[C@H]1OC(Oc2ccc3cnccc3c2)[C@@H](O)[C@@H](O)[C@@H]1O] |
| 148 | 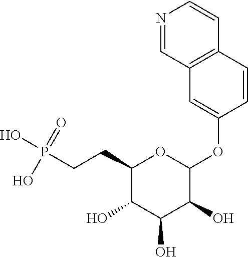 [SMILES: O=P(O)(O)CC[C@H]1OC(Oc2ccc3ccncc3c2)[C@@H](O)[C@@H](O)[C@@H]1O] |
| 149 | 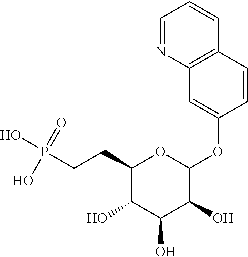 [SMILES: O=P(O)(O)CC[C@H]1OC(Oc2ccc3cccnc3c2)[C@@H](O)[C@@H](O)[C@@H]1O] |
| 150 | 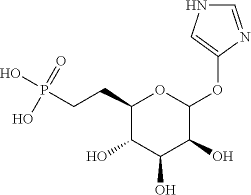 [SMILES: O=P(O)(O)CC[C@H]1OC(Oc2c[nH]cn2)[C@@H](O)[C@@H](O)[C@@H]1O] |
| 151 | 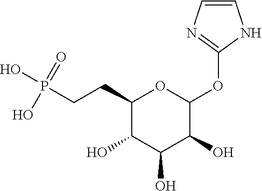 [SMILES: O=P(O)(O)CC[C@H]1OC(Oc2ncc[nH]2)[C@@H](O)[C@@H](O)[C@@H]1O] |
| 152 | 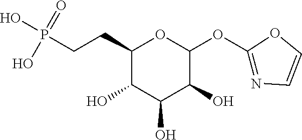 [SMILES: O=P(O)(O)CC[C@H]1OC(Oc2ncco2)[C@@H](O)[C@@H](O)[C@@H]1O] |
| 153 | 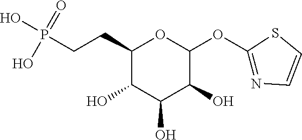 [SMILES: O=P(O)(O)CC[C@H]1OC(Oc2nccs2)[C@@H](O)[C@@H](O)[C@@H]1O] |
| 154 | 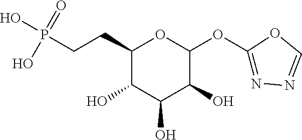 [SMILES: O=P(O)(O)CC[C@H]1OC(Oc2nnco2)[C@@H](O)[C@@H](O)[C@@H]1O] |
| 155 | 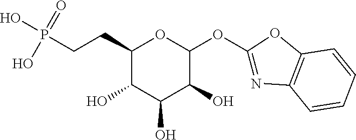 [SMILES: O=P(O)(O)CC[C@H]1OC(Oc2nc3ccccc3o2)[C@@H](O)[C@@H](O)[C@@H]1O] |
| 156 | 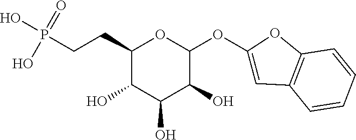 [SMILES: O=P(O)(O)CC[C@H]1OC(Oc2cc3ccccc3o2)[C@@H](O)[C@@H](O)[C@@H]1O] |
| 157 | 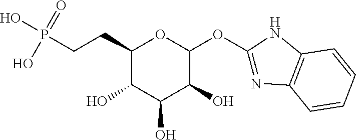 [SMILES: O=P(O)(O)CC[C@H]1OC(Oc2nc3ccccc3[nH]2)[C@@H](O)[C@@H](O)[C@@H]1O] |
| 158 | 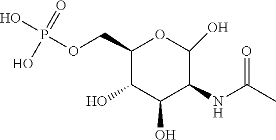 [SMILES: CC(=O)N[C@@H]1C(O)O[C@H](COP(=O)(O)O)[C@@H](O)[C@@H]1O] |
| 159 | 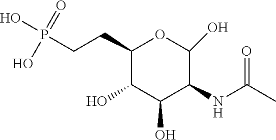 [SMILES: CC(=O)N[C@@H]1C(O)O[C@H](CCP(=O)(O)O)[C@@H](O)[C@@H]1O] |
| 160 | 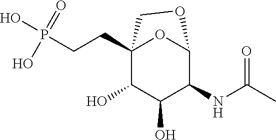 [SMILES: CC(=O)N[C@@H]1[C@H]2OC[C@](CCP(=O)(O)O)(O2)[C@@H](O)[C@@H]1O] |
| 161 | 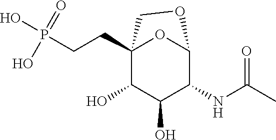 [SMILES: CC(=O)N[C@H]1[C@H]2OC[C@](CCP(=O)(O)O)(O2)[C@@H](O)[C@@H]1O] |
| 162 | 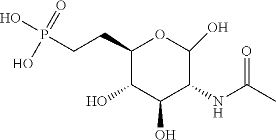 [SMILES: CC(=O)N[C@H]1C(O)O[C@H](CCP(=O)(O)O)[C@@H](O)[C@@H]1O] |
| 163 | 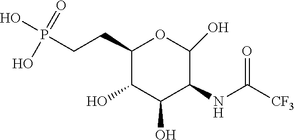 [SMILES: O=C(N[C@@H]1C(O)O[C@H](CCP(=O)(O)O)[C@@H](O)[C@@H]1O)C(F)(F)F] |
| 164 | 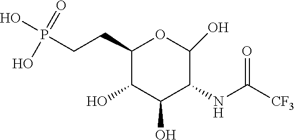 [SMILES: O=C(N[C@H]1C(O)O[C@H](CCP(=O)(O)O)[C@@H](O)[C@@H]1O)C(F)(F)F] |
| 165 | 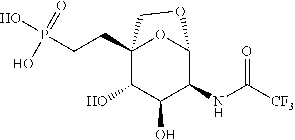 [SMILES: O=C(N[C@@H]1[C@H]2OC[C@](CCP(=O)(O)O)(O2)[C@@H](O)[C@@H]1O)C(F)(F)F] |
| 166 | 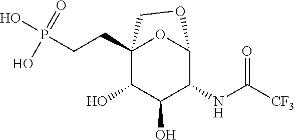 [SMILES: O=C(N[C@H]1[C@H]2OC[C@](CCP(=O)(O)O)(O2)[C@@H](O)[C@@H]1O)C(F)(F)F] |
| 167 | 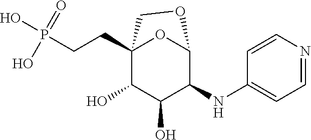 [SMILES: O=P(O)(O)CC[C@@]12CO[C@@H](O1)[C@@H](Nc1ccncc1)[C@@H](O)[C@@H]2O] |
| 168 | 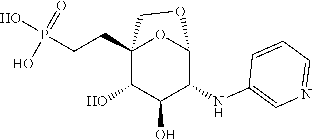 [SMILES: O=P(O)(O)CC[C@@]12CO[C@@H](O1)[C@H](Nc1cccnc1)[C@@H](O)[C@@H]2O] |
| 169 | 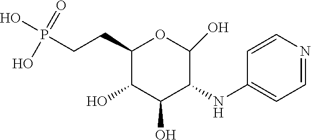 [SMILES: O=P(O)(O)CC[C@H]1OC(O)[C@H](Nc2ccncc2)[C@@H](O)[C@@H]1O] |
| 170 | 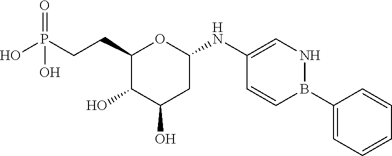 [SMILES: O=P(O)(O)CC[C@H]1O[C@H](NC2=CNB(c3ccccc3)C=C2)C[C@@H](O)[C@@H]1O] |

### X. Processes of Manufacture

The extracellular protein degrading compounds of the present invention can be manufactured according to routes described in the Working Examples below or as otherwise known in the patent or scientific literature and if appropriate supported by the knowledge of the ordinary worker or common general knowledge.

Some of the carbons in the extracellular protein degrading compounds described herein are drawn with designated stereochemistry. Other carbons are drawn without stereochemical designation. When drawn without designated stereochemistry, that carbon can be in any desired stereochemical configuration that achieves the desired purpose. One skilled in the art will recognize that pure enantiomers, enantiomerically enriched compounds, racemates and diastereomers can be prepared by methods known in the art as guided by the information provided herein. Examples of methods to obtain optically active materials include at least the following:
i) chiral liquid chromatography—a technique whereby diastereomers are separated in a liquid mobile phase by virtue of their differing interactions with a stationary phase (including vial chiral HPLC). The stationary phase can be made of chiral material or the mobile phase can contain an additional chiral material to provoke the differing interactions;
ii) non-chiral chromatography of diastereomers-Often diastereomers can be separated using normal non-chiral column conditions;
iii) chiral gas chromatography—a technique whereby the racemate is volatilized and enantiomers are separated by virtue of their differing interactions in the gaseous mobile phase with a column containing a fixed non-racemic chiral adsorbent phase;
iv) simultaneous crystallization—a technique whereby the individual diastereomers are separately crystallized from a solution;
v) enzymatic resolutions—a technique whereby partial or complete separation of diastereomers are separated by virtue of differing rates of reaction with an enzyme;
vi) chemical asymmetric synthesis—a synthetic technique whereby the desired diastereomer is synthesized from an achiral precursor under conditions that produce asymmetry (i.e. chirality) in the product, which may be achieved by chiral catalysts or chiral auxiliaries;
vii) diastereomer separations—a technique whereby a racemic compound is reaction with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer; and
viii) extraction with chiral solvents—a technique whereby diastereomers are separated by virtue of preferential dissolution of one over the others in a particular chiral solvent.

### Example 1. Synthesis of Compounds of the Present Invention

The compounds of the present invention can be synthesized by a variety of methods. Non-limiting examples of synthetic methods include those shown in the schemes below.

### Synthesis 1-1: Synthesis of Compounds Via Serial Nucleophilic Substitution

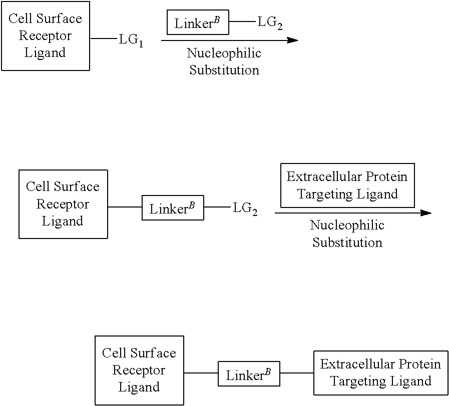

In certain embodiments the Compound of Formula I is synthesized by a series of nucleophilic substitutions. Depending on the identity of the nucleophile, leaving groups, and other functional groups on the molecule these reactions can take place in the absence of protecting groups or can have one or more protecting and deprotecting reactions. In certain embodiments LG₂ is protected before the first nucleophilic substation reaction and then deprotected before the second nucleophilic substation reaction.

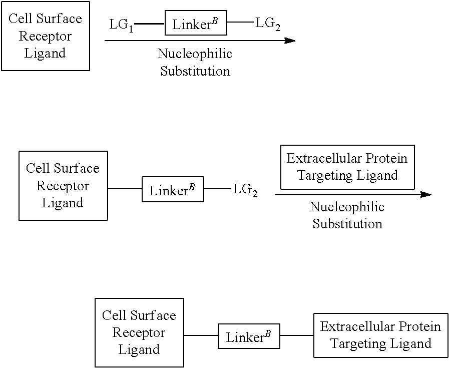

The leaving group can be either on the Linker or on the cell surface receptor ligand. For example when Linker^{A} is CH₂—OH the leaving group can be a halogen, tosylate, mesylate, acyl chloride, ester, etc. Control of which leaving group gets displaced first can be achieved by choosing specific nucleophiles, leaving groups, or using protecting groups.

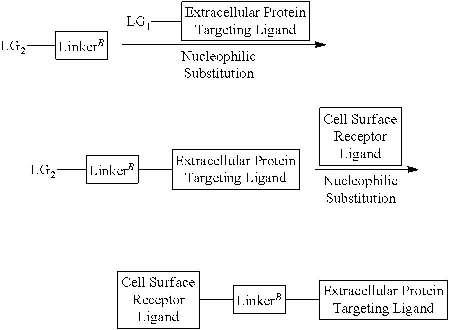

The order of addition can also be varied to achieve the desired synthetic goal. For example, the Linker and Extracellular Protein Targeting Ligand can be synthesized first and then the Cell Surface Receptor Ligand added after if desired.

### Synthesis 1-2: Synthesis of Compounds Via Other Transformations

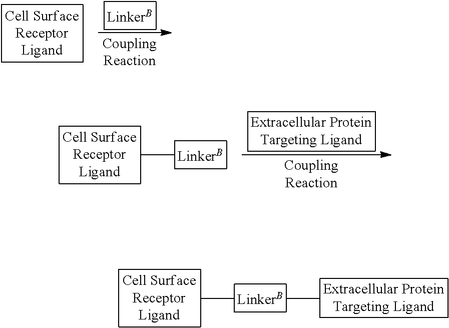

In certain embodiments coupling reactions are used to form bonds between the Cell Surface Receptor Ligand, Linker^{B} and the Extracellular Protein Targeting Ligand. Non-limiting examples of coupling reactions include peptide coupling, the Buchwald Hartwig Reaction, Fukuyama coupling, Gomberg-Bachmann reaction, Cardiot-Chodkiewicz coupling, Castro-Stephens coupling, Corey-House coupling, Cassar reaction, Kumada coupling, Heck reaction, Sonogashira coupling, Negishi coupling, Stille cross coupling, Suzuki reaction, Hiyama coupling, and Liebeskind-Strogi coupling

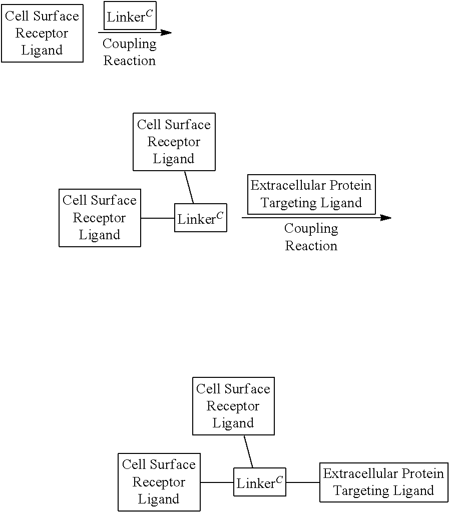

The synthetic steps described herein can be applied to mono-, bis-, and tri- versions of the compounds of the present invention.

### Synthesis 1-3: Additional Synthetic Methods

Compounds can be synthesized with mixtures of the methods described herein. For example, the bond between Cell Surface Receptor and Linker^{B} may be formed by a Michael addition while the bond between Linker^{B} and Extracellular Protein Targeting Ligand may be formed by nucleophilic addition. The skilled artisan will be able to adapt combinations of these schemes to synthesize compounds of the present invention.

Non-limiting examples of transformations that can be utilized in the synthesis of compounds of the present invention include:

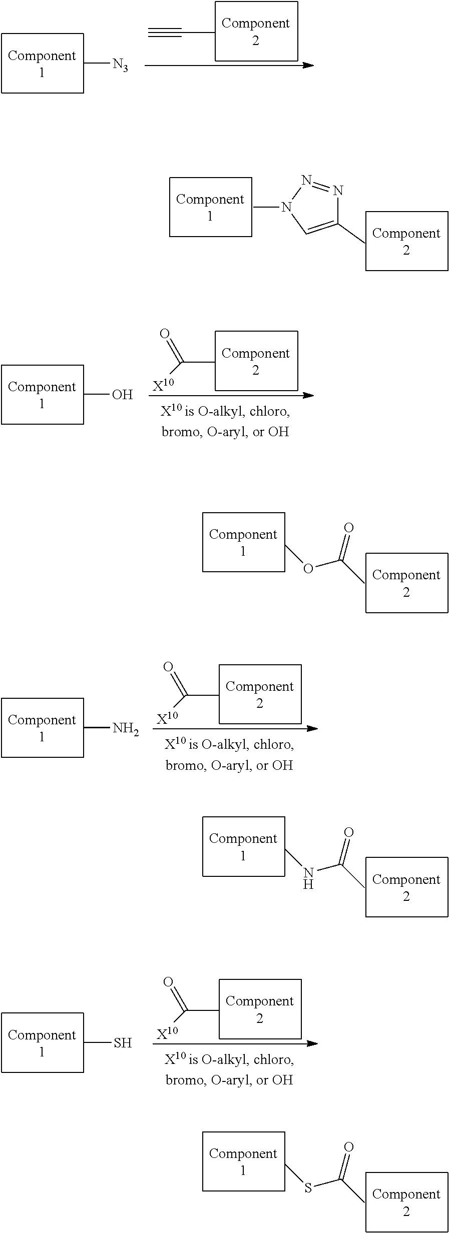

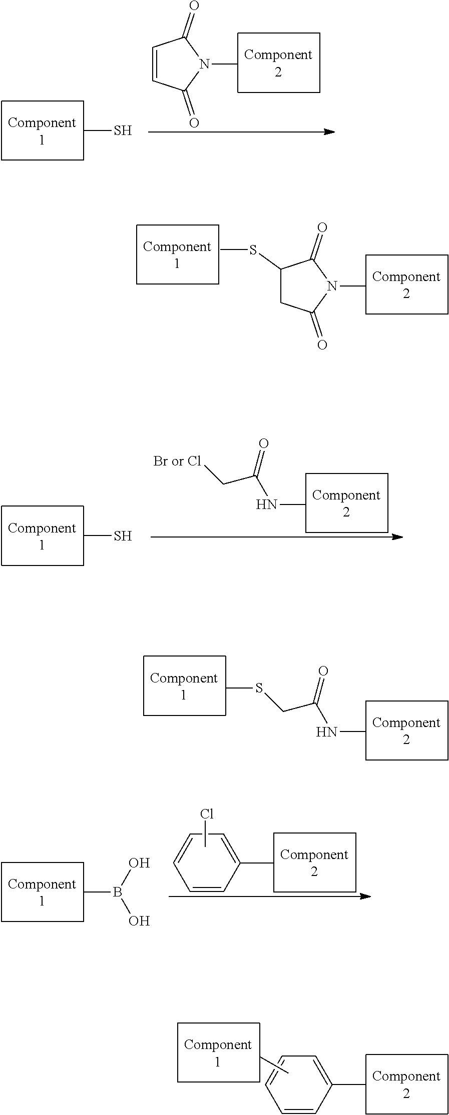

Non-limiting examples of Component 1 and Component 2 include:

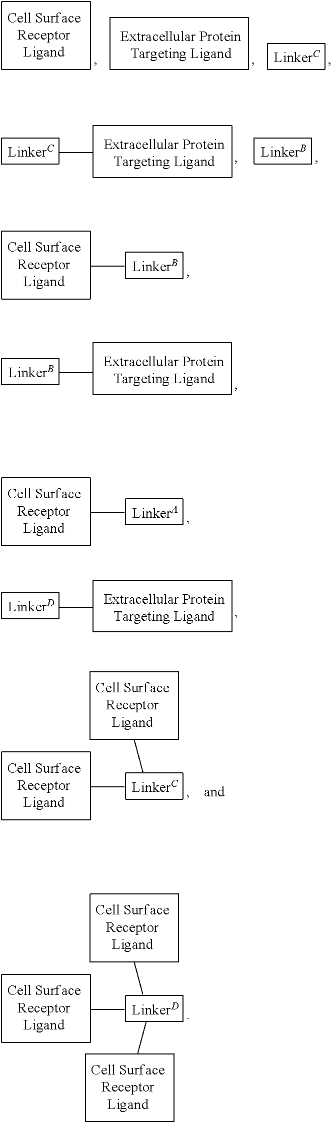

### Example 2. Synthesis of Mannose 6-Phosphate Receptor and ASGPR Ligands

Mannose 6-Phosphate Receptor Ligands and ASGPR Ligands can be synthesized using known chemical methodologies from commercially available or known starting materials. The following are non-limiting examples of how to synthesize Mannose 6-Phosphate Receptor Ligands and ASGPR Ligands. The skilled artisan will readily recognize modifications that can be used to make other compounds of the present invention.

### Synthesis 2-1: Synthesis of Imino and Carba Sugars

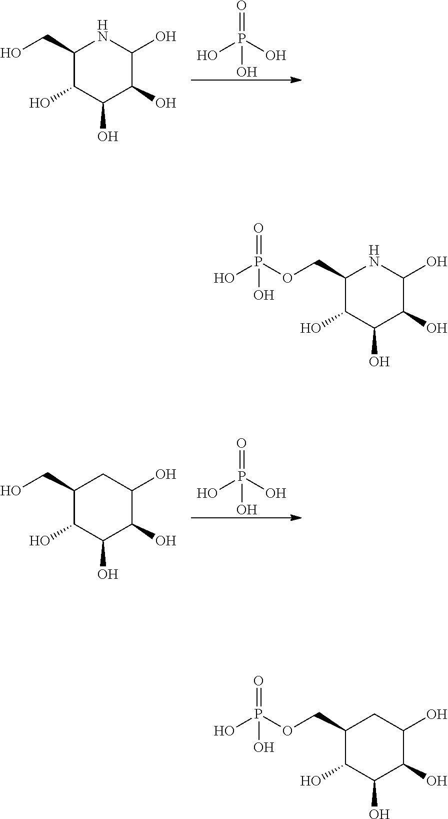 [SMILES: OC[C@H]1NC(O)[C@@H](O)[C@@H](O)[C@@H]1O ; O=P(O)(O)O ; O=P(O)(O)OC[C@H]1NC(O)[C@@H](O)[C@@H](O)[C@@H]1O ; OC[C@H]1CC(O)[C@@H](O)[C@@H](O)[C@@H]1O ; O=P(O)(O)O ; O=P(O)(O)OC[C@H]1CC(O)[C@@H](O)[C@@H](O)[C@@H]1O]

### Synthesis 2-2: Synthesis of R⁵ and R⁵⁵ Groups

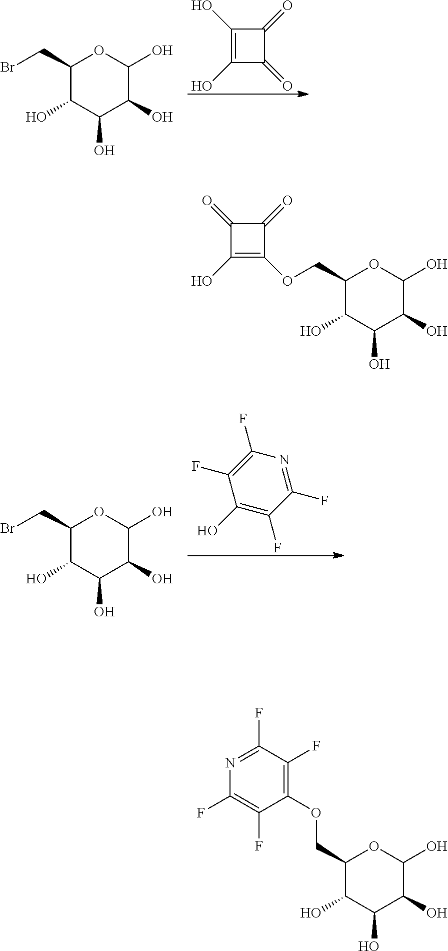 [SMILES: OC1O[C@H](CBr)[C@@H](O)[C@H](O)[C@@H]1O ; O=c1c(O)c(O)c1=O ; O=c1c(O)c(OC[C@H]2OC(O)[C@@H](O)[C@@H](O)[C@@H]2O)c1=O ; OC1O[C@H](CBr)[C@@H](O)[C@H](O)[C@@H]1O ; Oc1c(F)c(F)nc(F)c1F ; OC1O[C@H](COc2c(F)c(F)nc(F)c2F)[C@@H](O)[C@H](O)[C@@H]1O]

Various nucleophilic and electrophilic mannose 6-phosphate precursors are known. For example (3S,4S,5S,6S)-6-(bromomethyl)tetrahydro-2H-pyran-2,3,4,5-tetraol can be used to prepare several R⁵ and R⁵⁵ groups from nucleophilic reagents. Other useful starting materials that can be readily adapted to afford compounds of the present invention include:

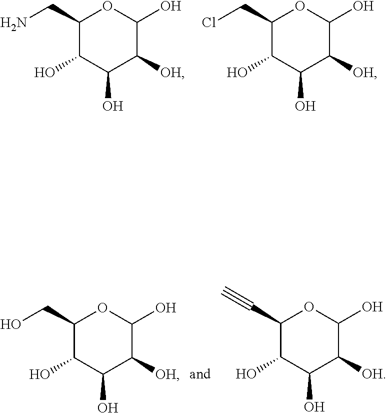

In certain aspects the difference in reactivity of the C6 functional group and the alcohols will be sufficient to allow installation of the desired R⁵ or R⁵⁵ group. In other embodiments an orthogonal protecting group can be used. For example methyl ethers can be installed and removed using conventional chemistry to protect the hydroxyls from undesired reactivity.

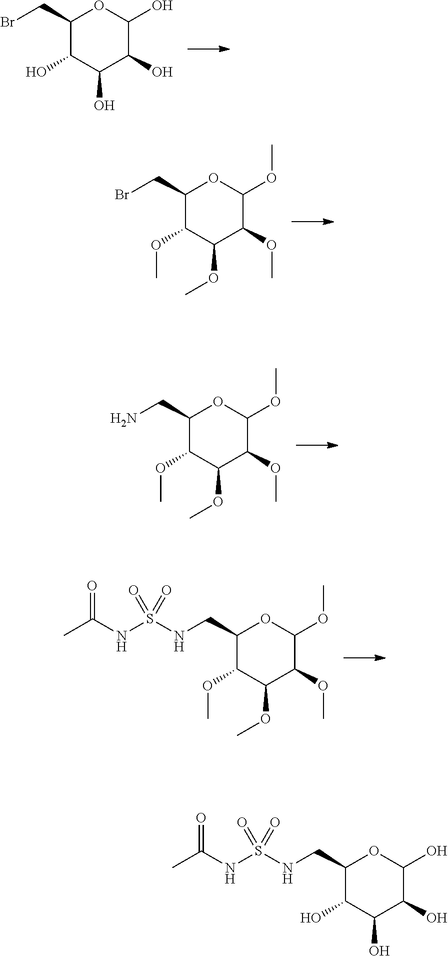

In certain embodiments the ASGPR Ligand is a ligand described in WO2021/155317 and the synthesis is provided therein. WO2021/155317 is incorporated by reference for all purposes.

### Synthesis 2-3. Preparation of Structure 1, 5, 9, 13, 33, 37, 41, 45, 49, 53

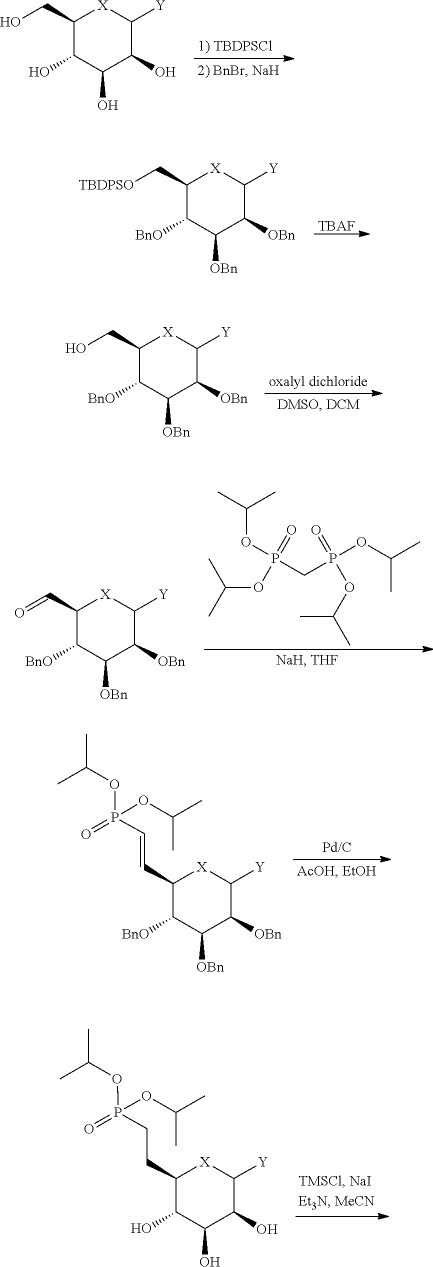

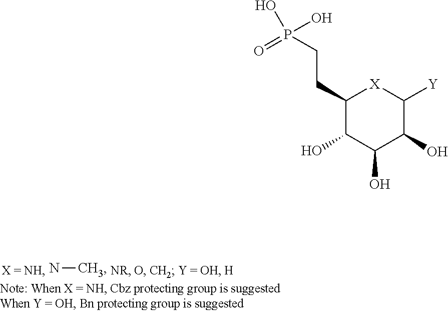

### Synthesis 2-4. Preparation of Structure 17, 25

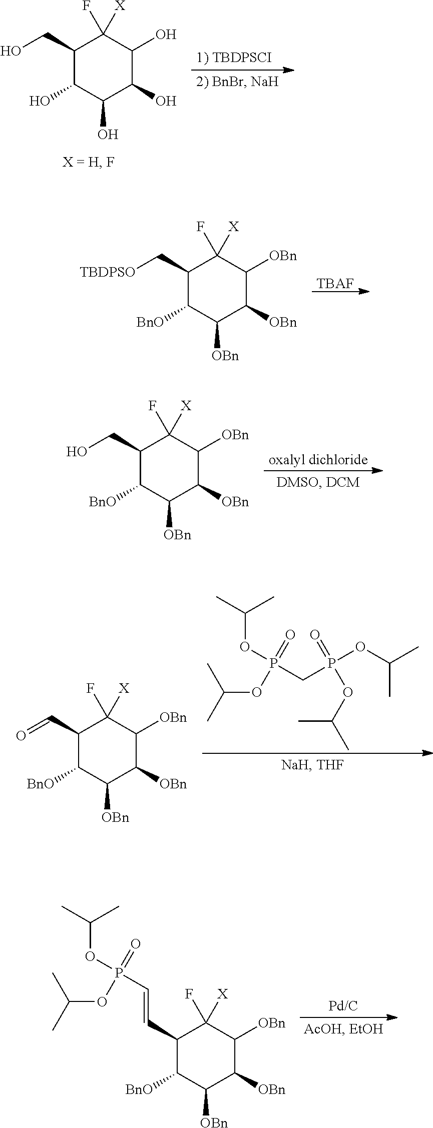

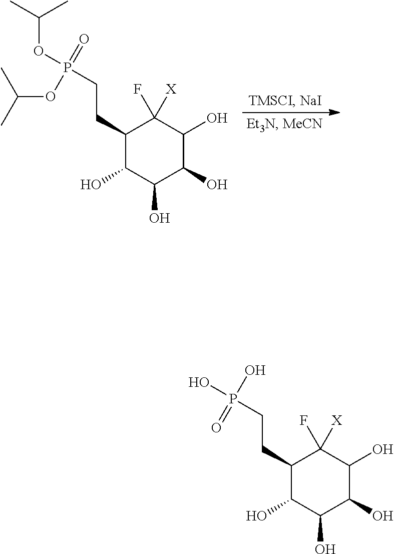

### Synthesis 2-5. Preparation of Structure 21, 29

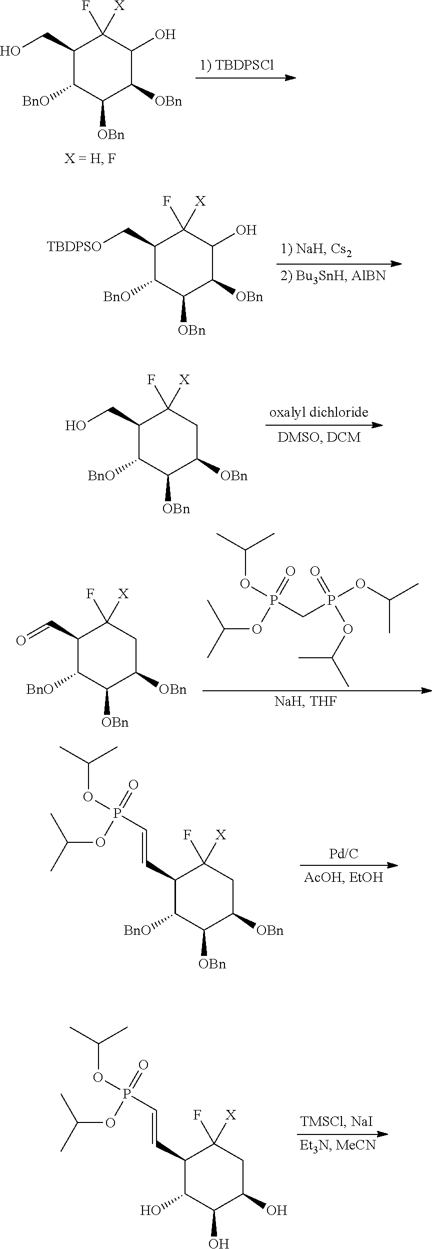

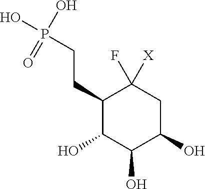

### Synthesis 2-6. Preparation of Structure 2, 6, 10, 14, 18, 22, 26, 30, 34, 38, 42, 46, 50, 54

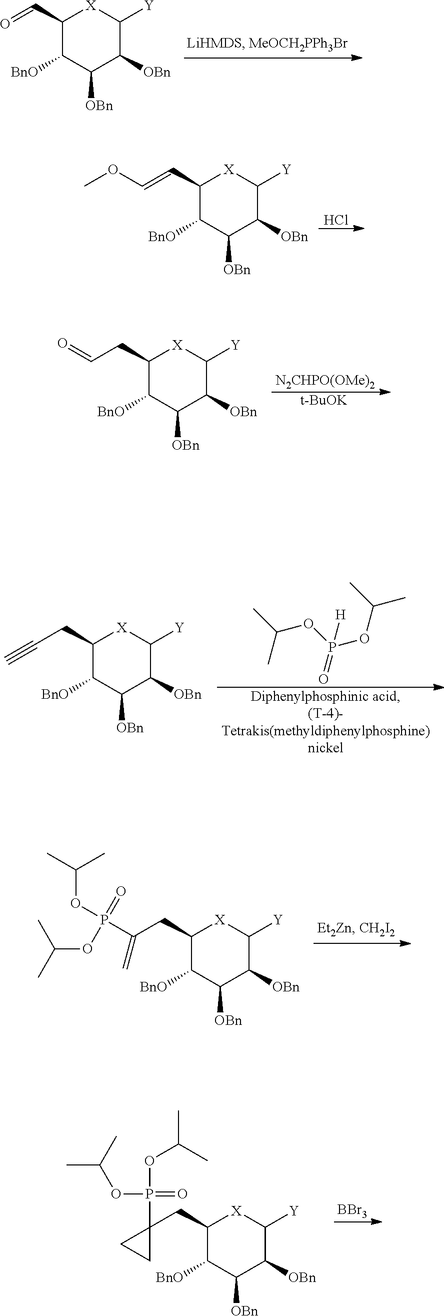

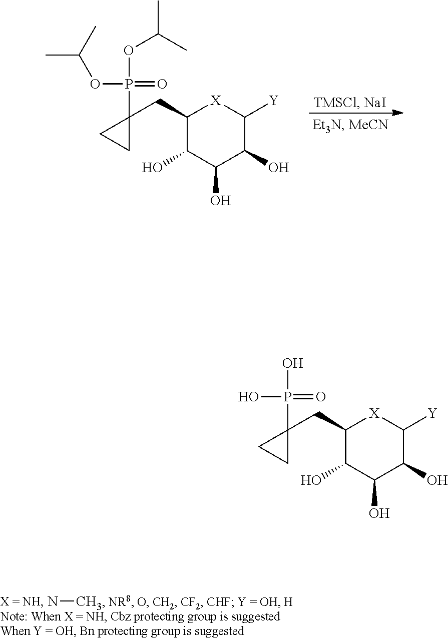

### Synthesis 2-7. Preparation of Structure 3, 7, 11, 15, 19, 23, 27, 31, 35, 39, 43, 47, 51, 55

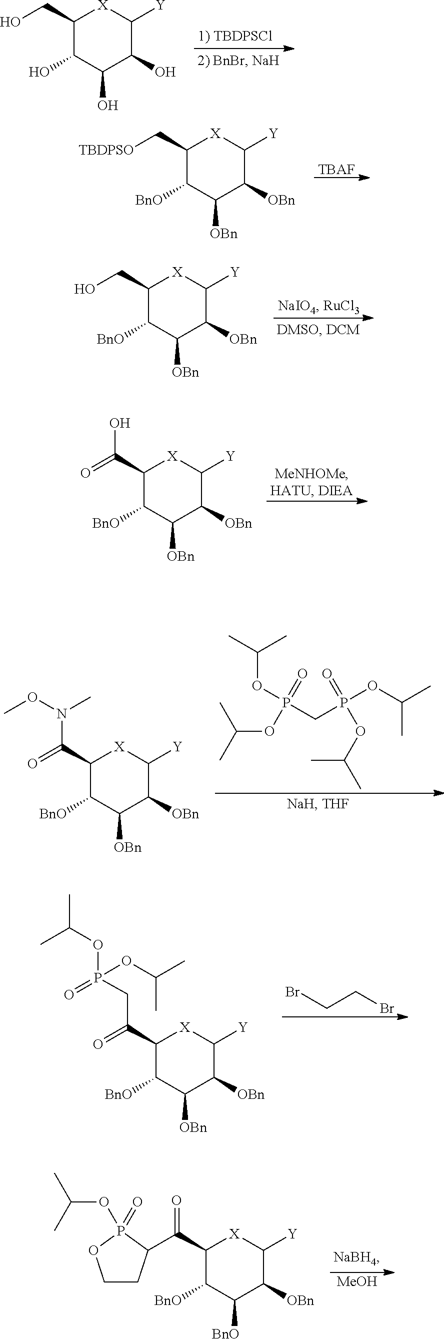

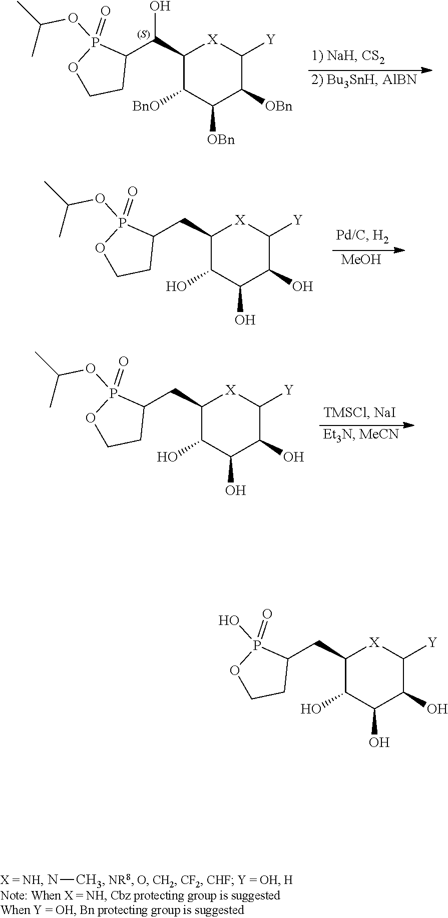

### Synthesis 2-8. Preparation of Structure 4, 8, 12, 16, 20, 24, 28, 32, 36, 40, 44, 48, 52, 56

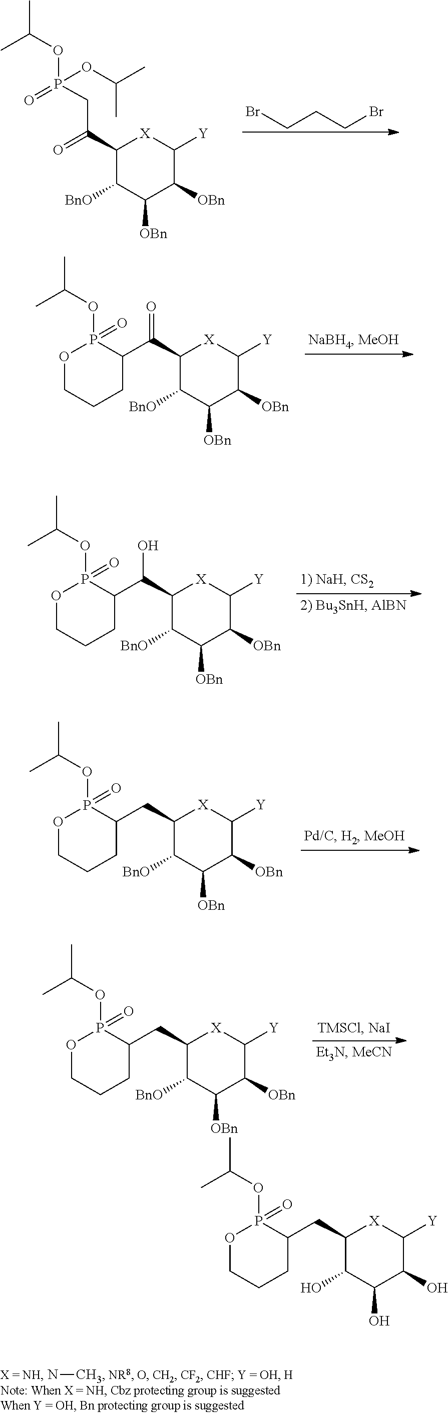

### Synthesis 2-9. Preparation of Structure 57

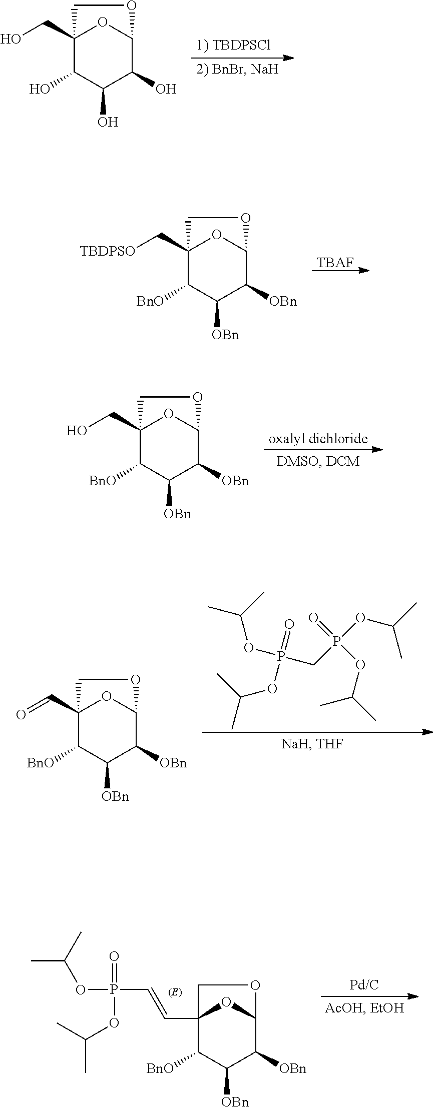

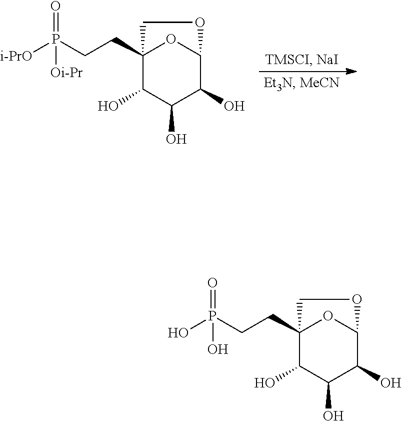

### Synthesis 2-10. Preparation of Structure 58

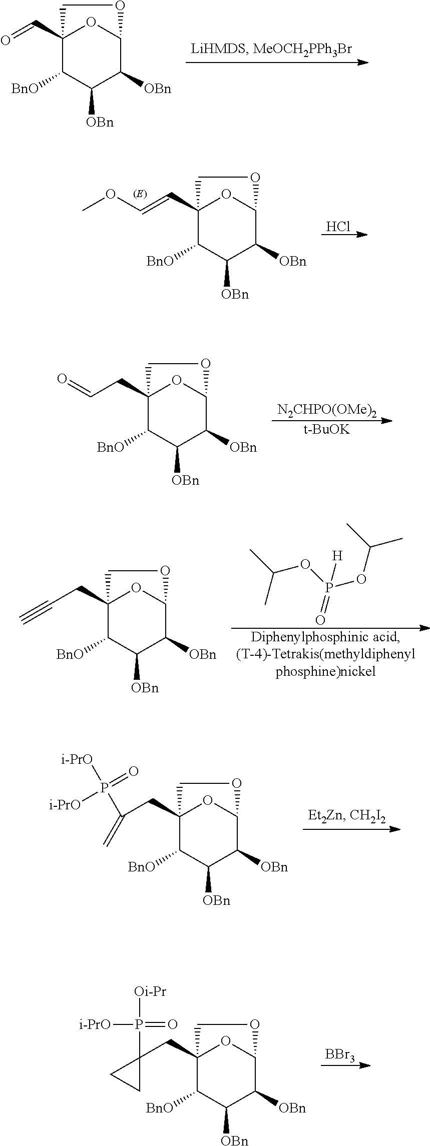

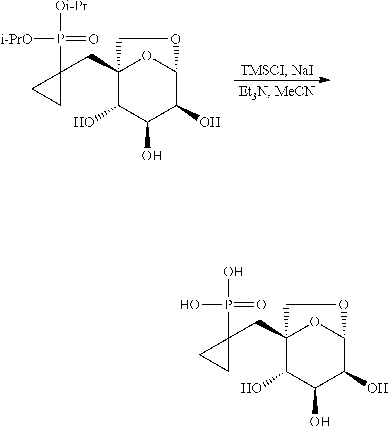

### Synthesis 2-11. Preparation of Structure 59

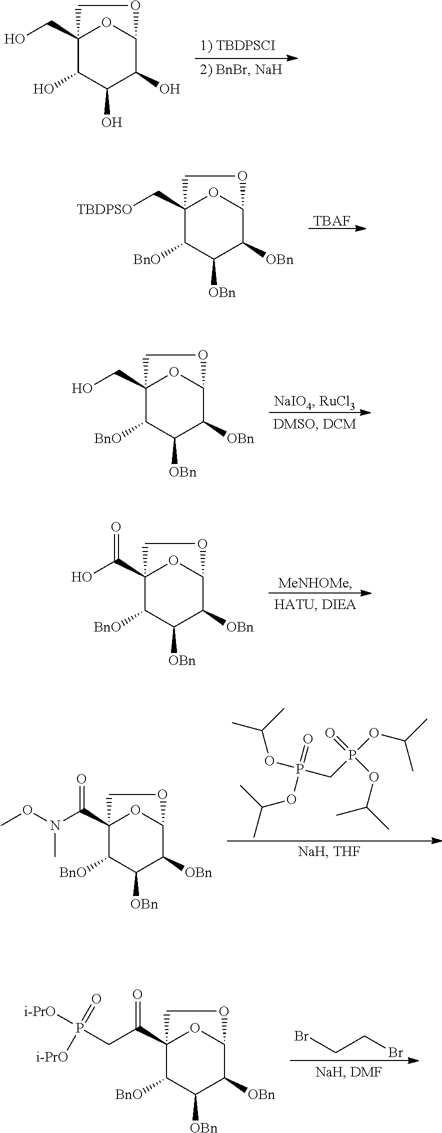

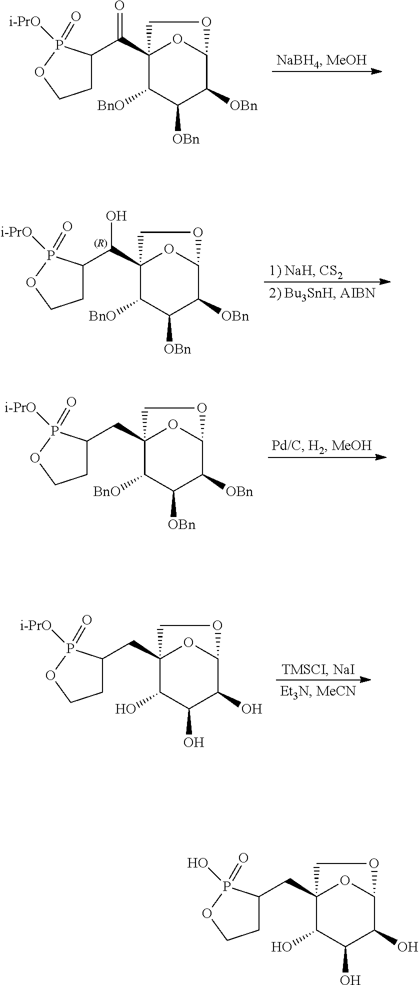

### Synthesis 2-12. Preparation of Structure 60

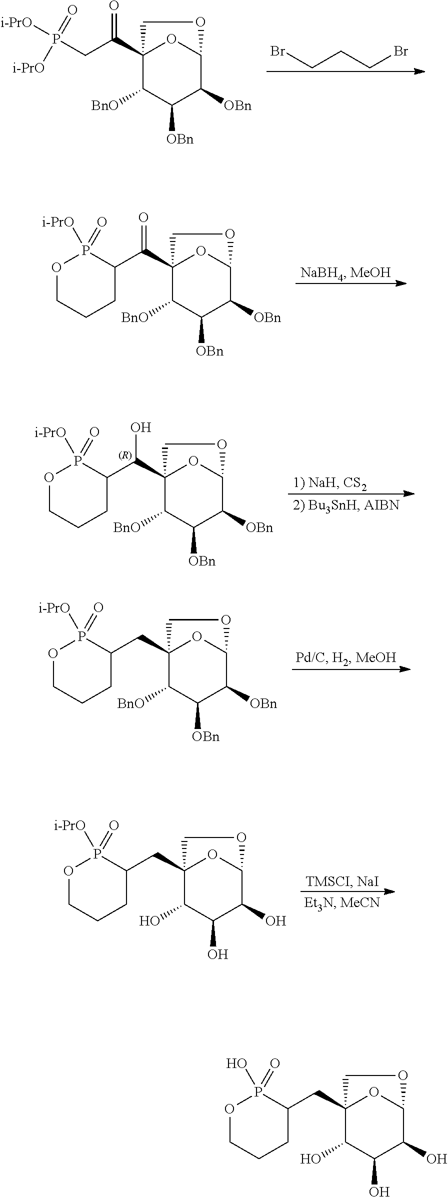

### Synthesis 2-13. Preparation of Structure 61

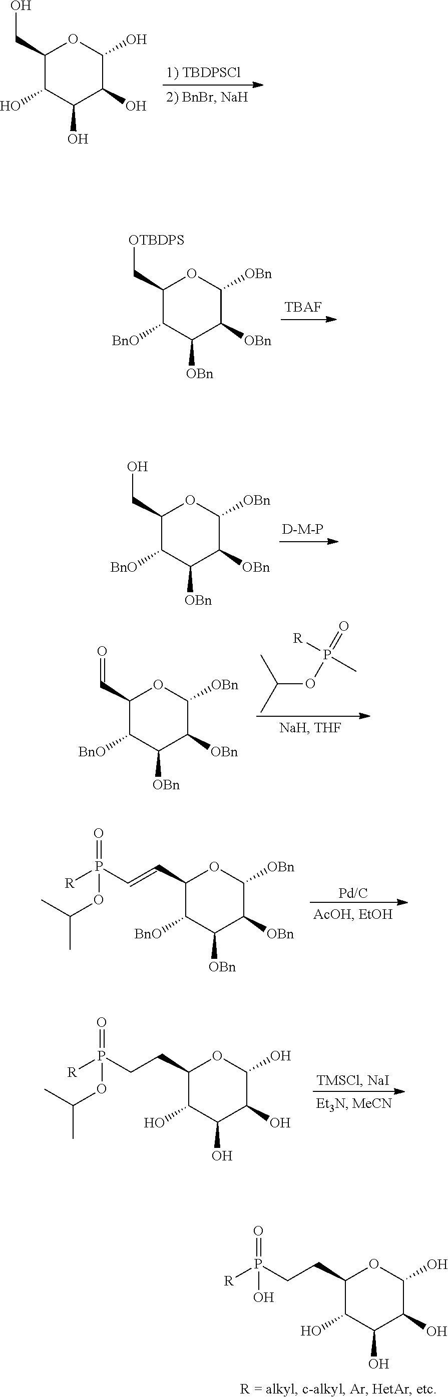

### Synthesis 2-14. Preparation of Structure 62

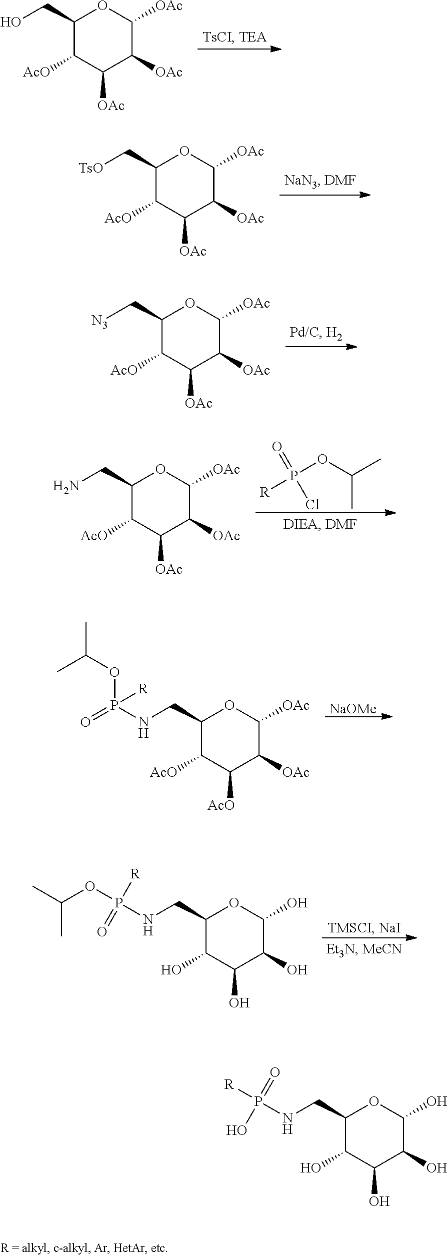

### Synthesis 2-15. Preparation of Structure 63 PGP-664C3

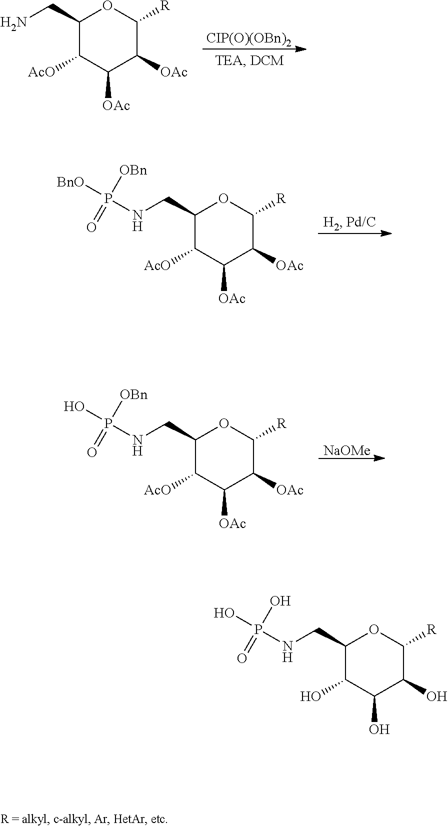

### Synthesis 2-16. Preparation of Structure 64, 65

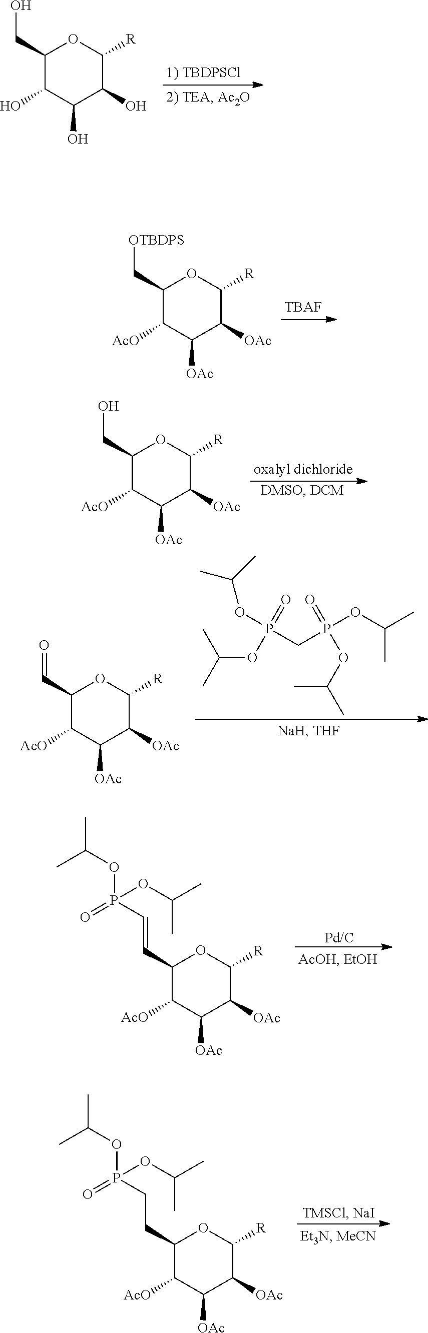

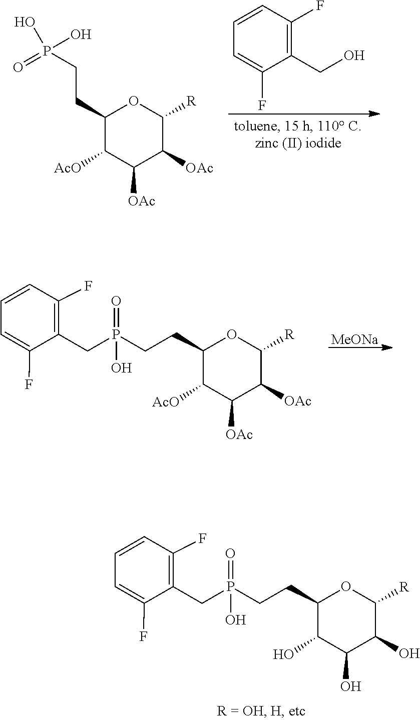

### Synthesis 2-17. Preparation of Structure 66

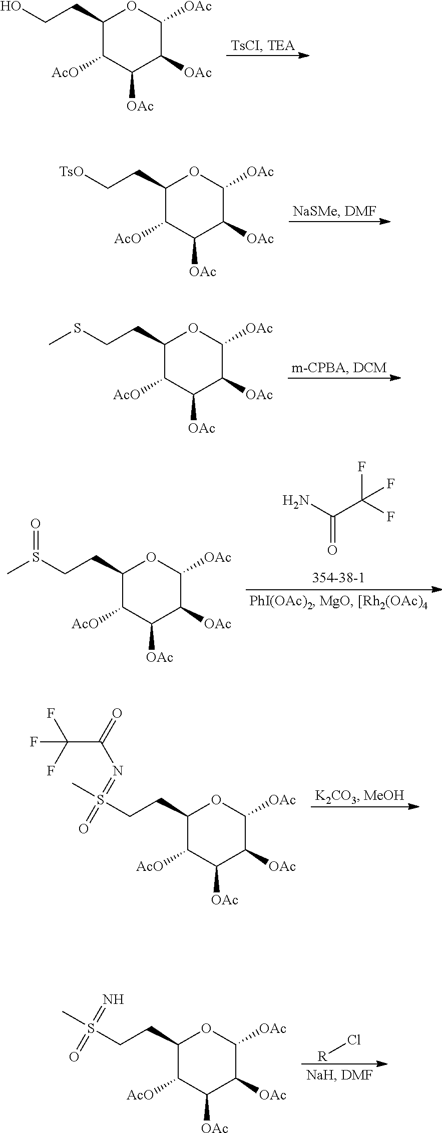

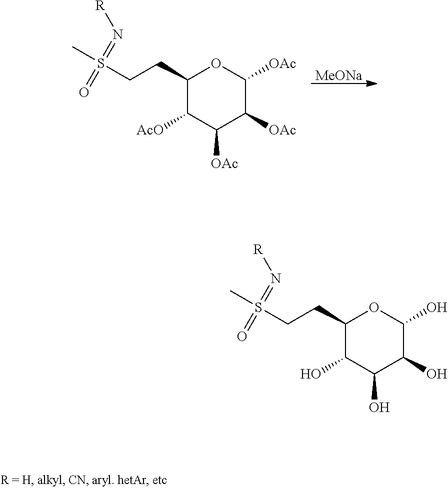

### Synthesis 2-18. Preparation of Structure 67

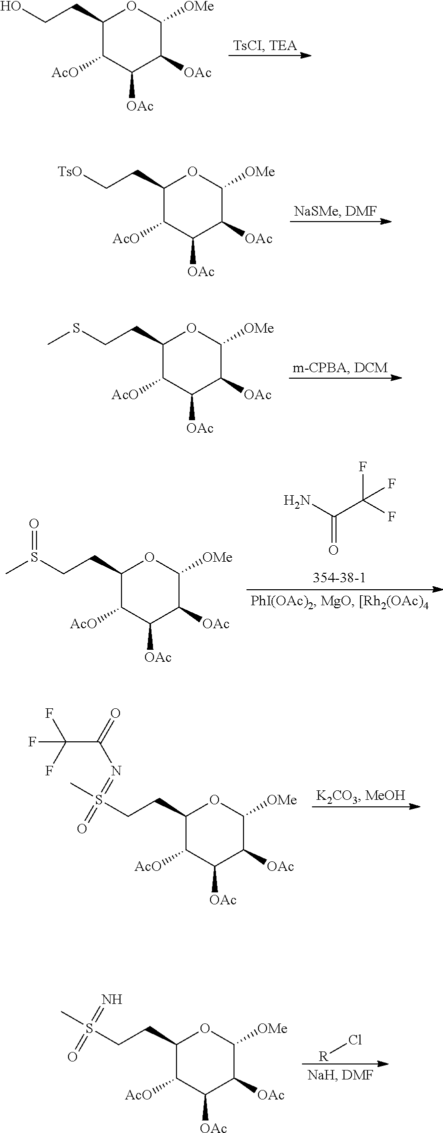

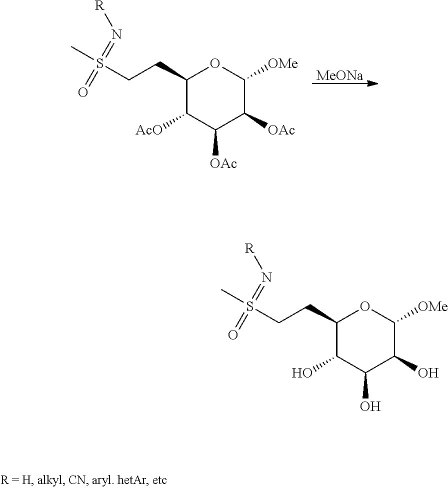

### Synthesis 2-19. Preparation of Structure 68

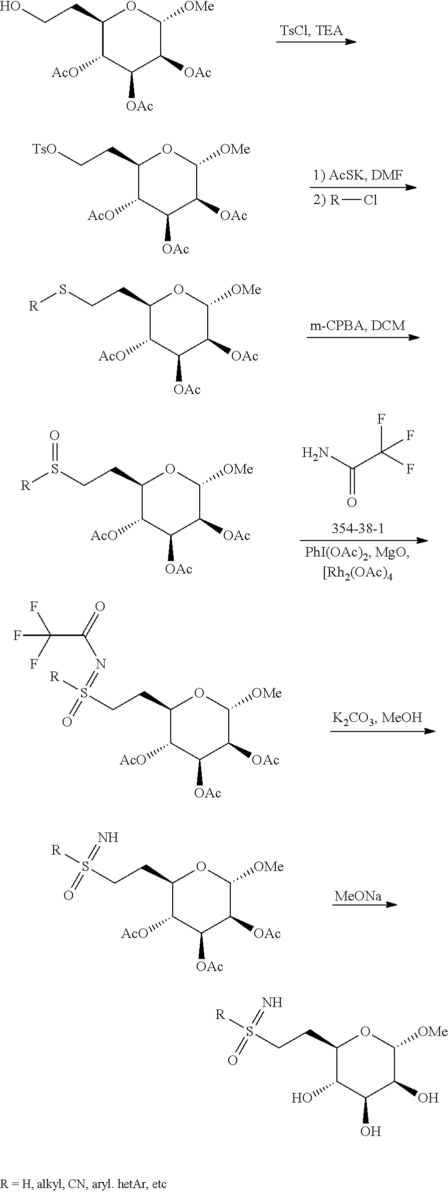

### Synthesis 2-20. Preparation of Structure 69

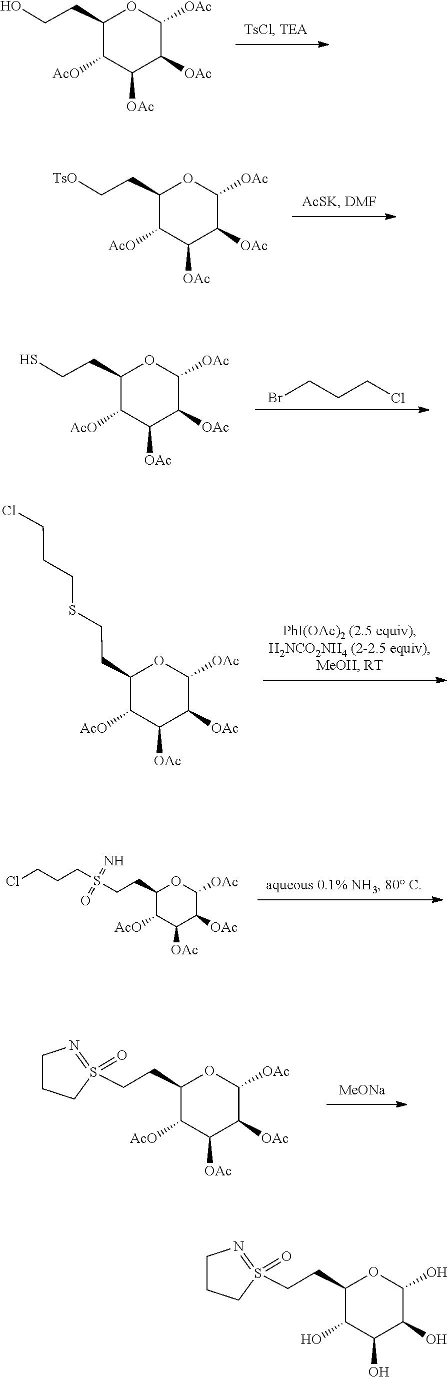

### Synthesis 2-21. Preparation of Structure 70

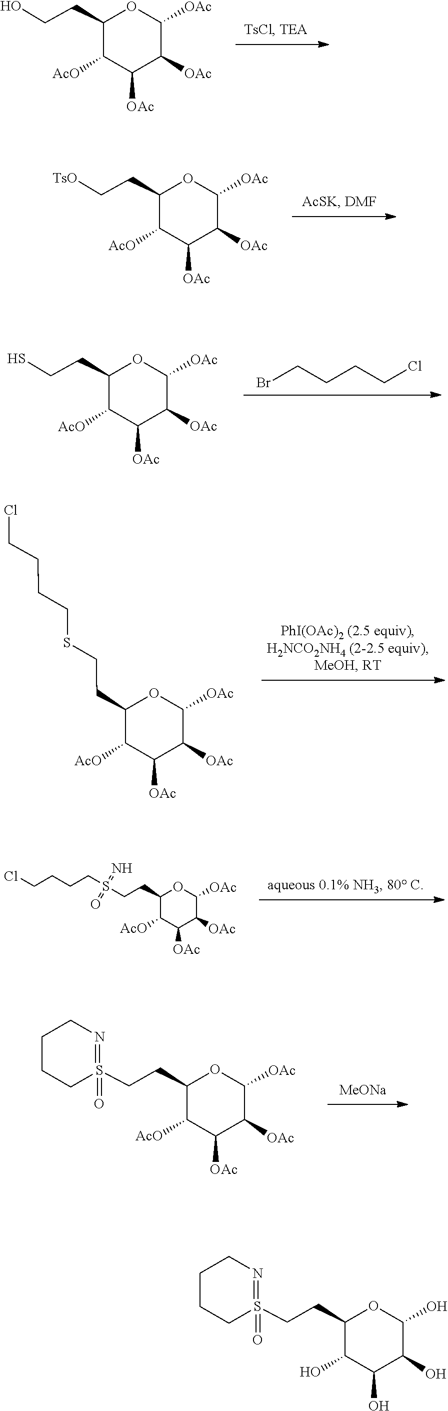

### Synthesis 2-22. Preparation of Structure 71

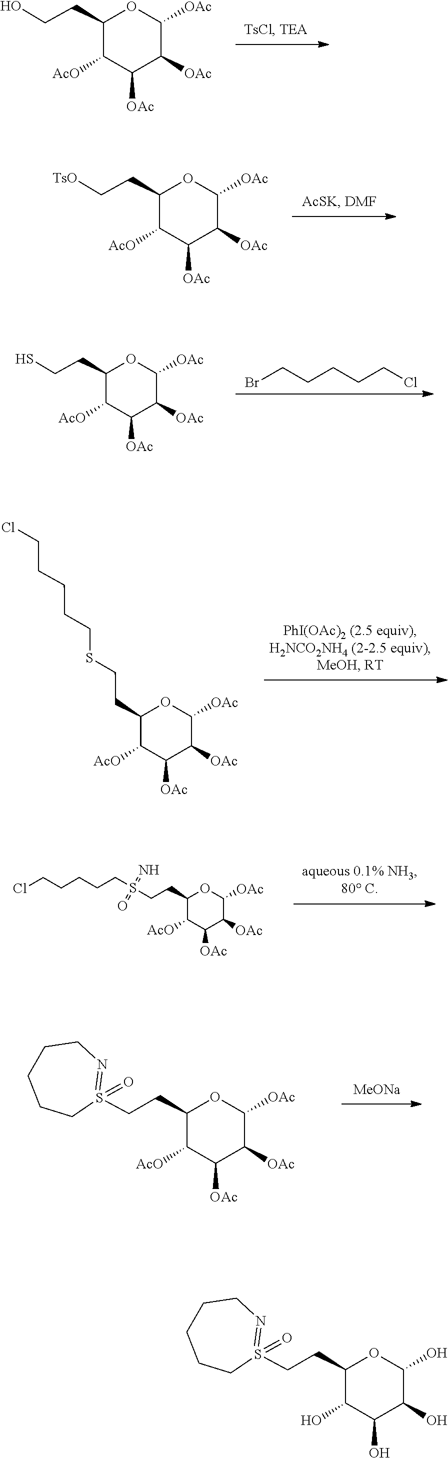

### Synthesis 2-23. Preparation of Structure 72 PGP-6′C3

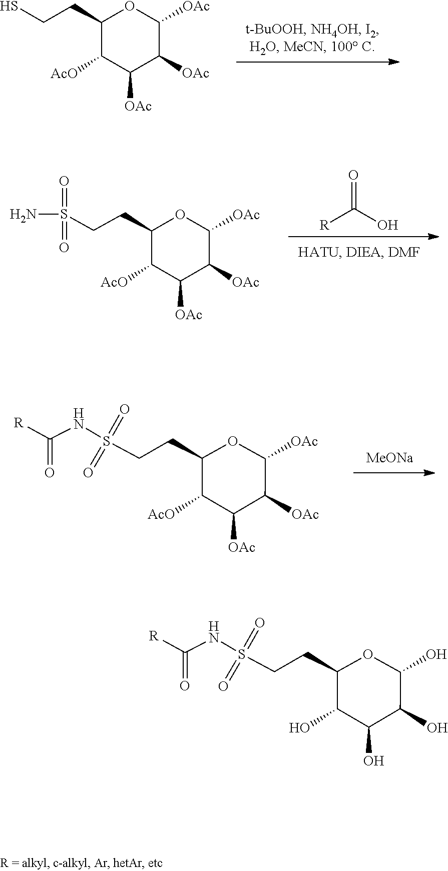

### Synthesis 2-24. Preparation of Structure 73

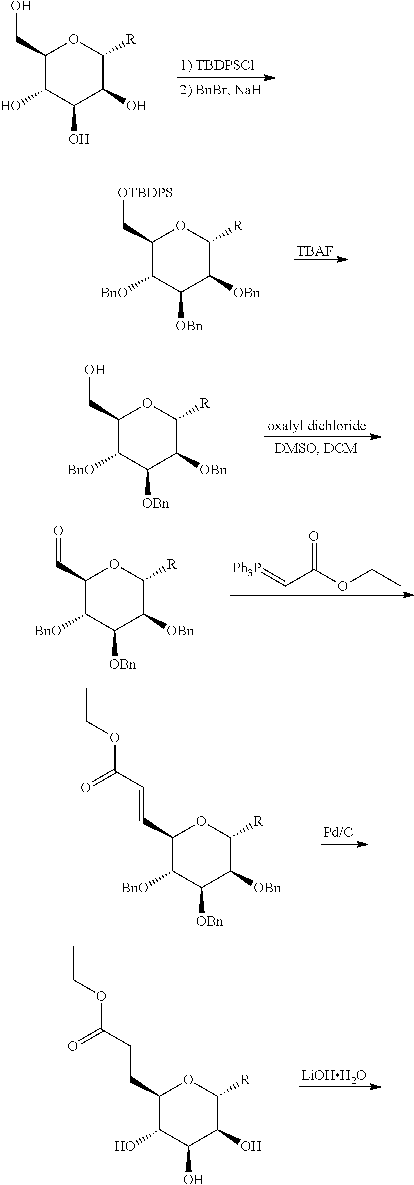

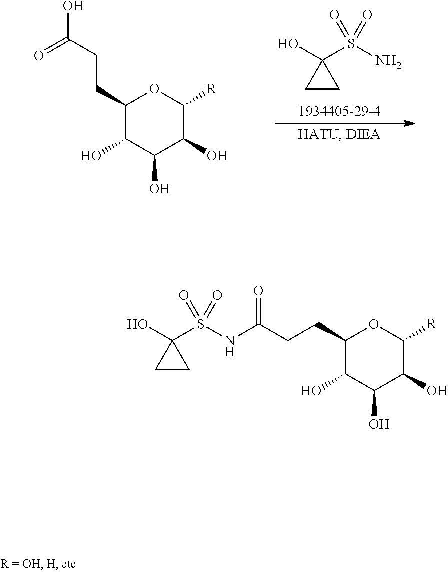

### Synthesis 2-25. Preparation of Structure 74

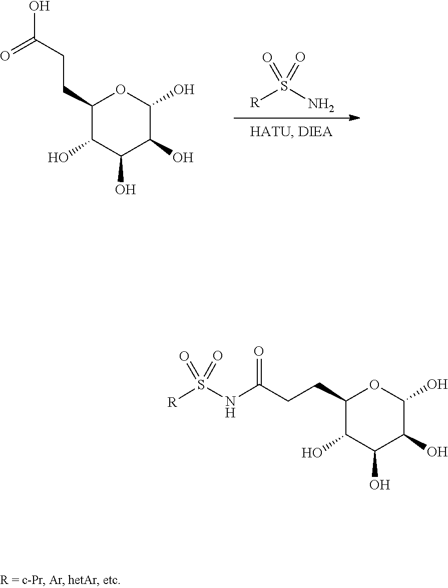

### Synthesis 2-26. Preparation of Structure 75

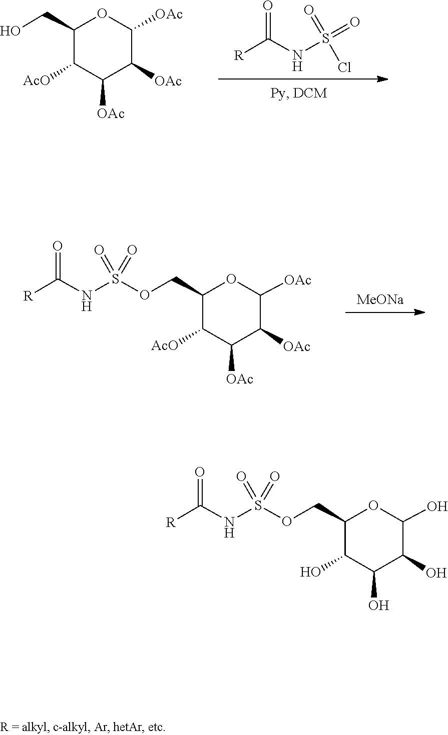

### Synthesis 2-27. Preparation of Structure 76

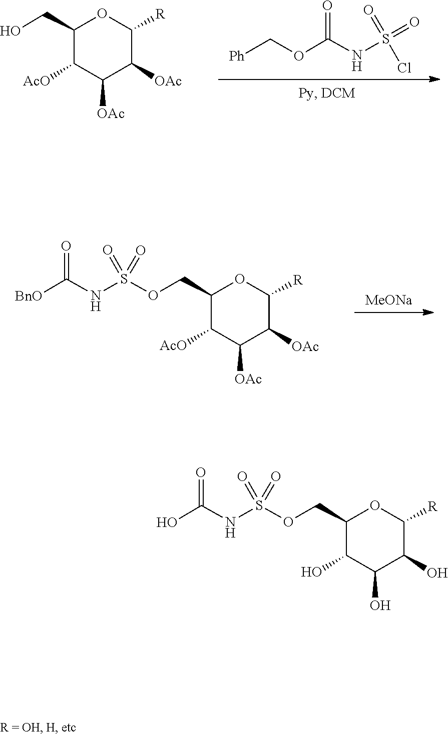

### Synthesis 2-28. Preparation of Structure 77

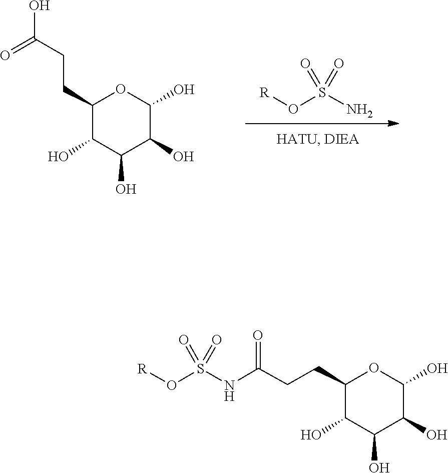

### Synthesis 2-29. Preparation of Structure 78

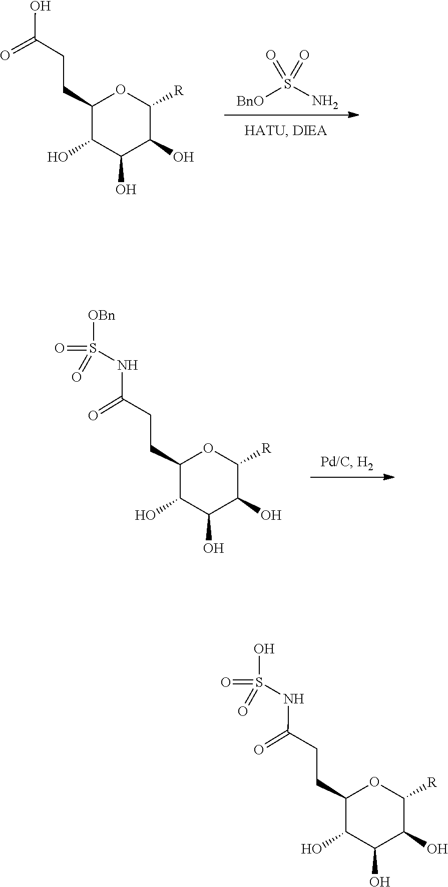

### Synthesis 2-30. Preparation of Structure 79

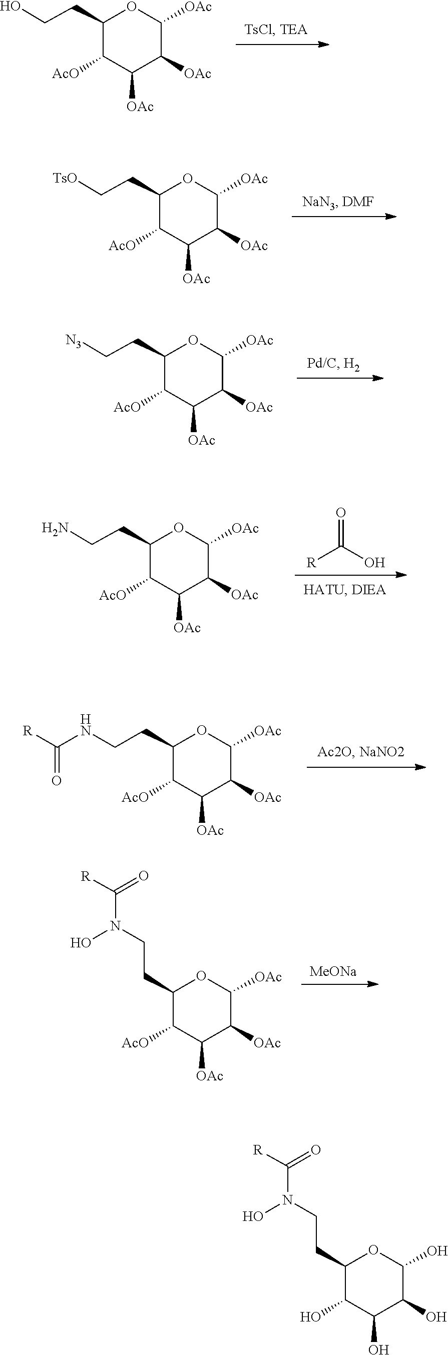

### Synthesis 2-31. Preparation of Structure 80

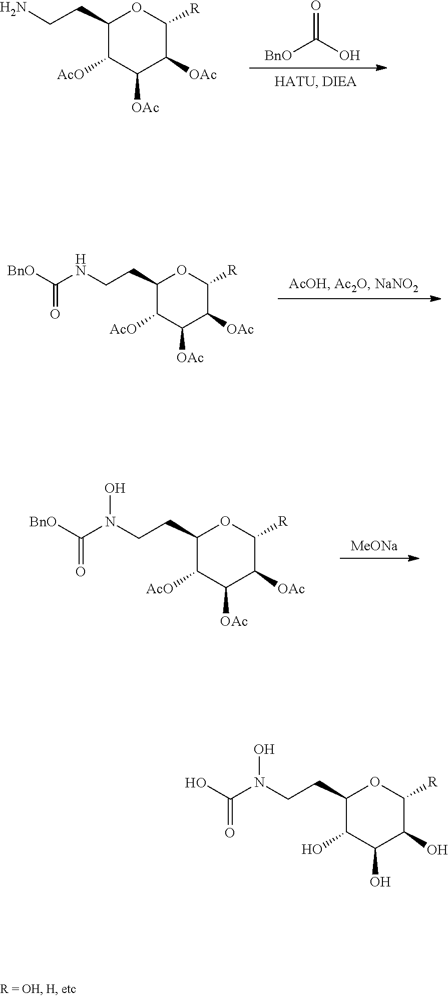

### Synthesis 2-32. Preparation of Structure 81

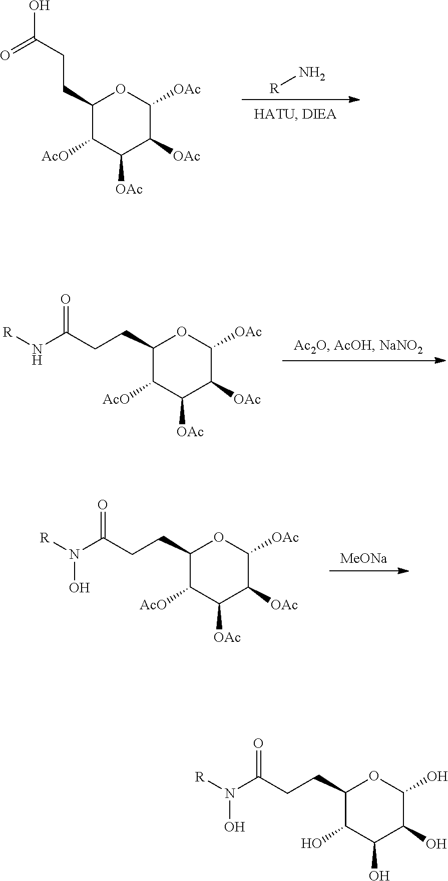

### Synthesis 2-33. Preparation of Structure 82

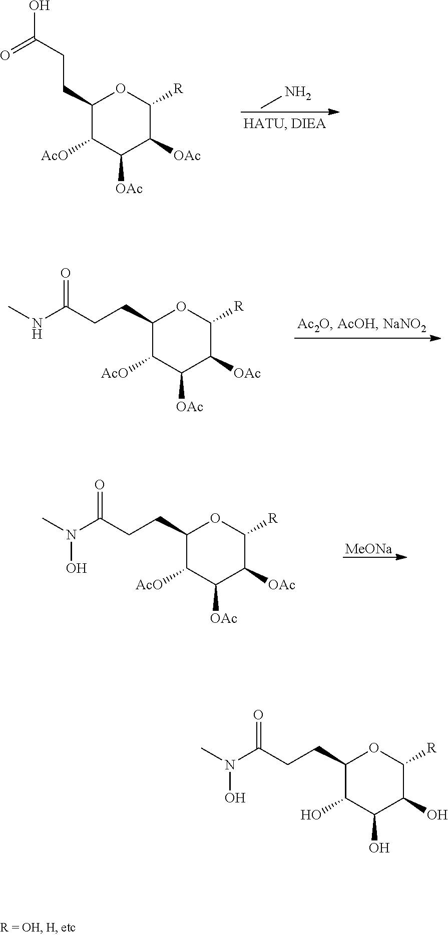

### Synthesis 2-34. Preparation of Structure 83

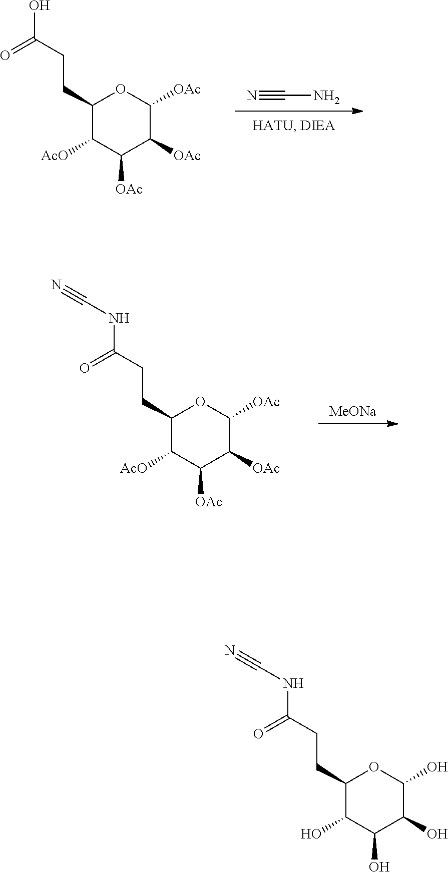

### Synthesis 2-35. Preparation of Structure 84

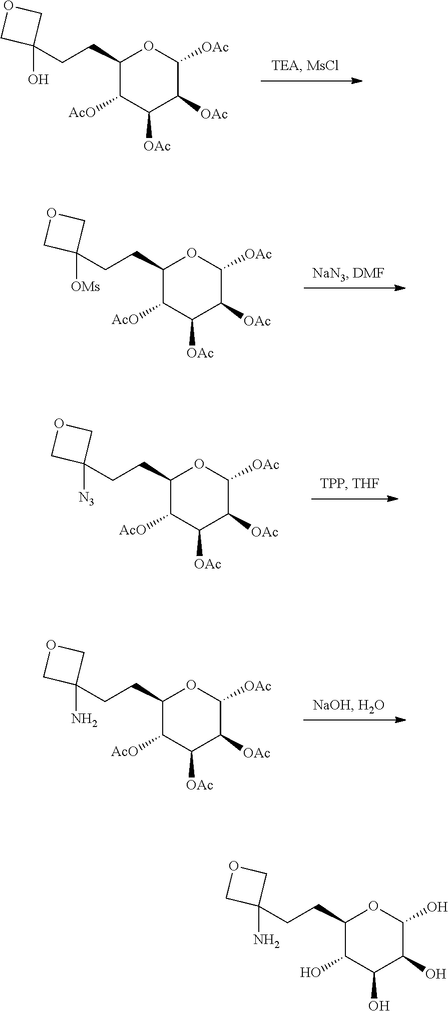 [SMILES: CC(=O)O[C@H]1O[C@H](CCC2(O)COC2)[C@@H](OC(C)=O)[C@H](OC(C)=O)[C@@H]1OC(C)=O ; CC(=O)O[C@H]1O[C@H](CCC2(OS(C)(=O)=O)COC2)[C@@H](OC(C)=O)[C@H](OC(C)=O)[C@@H]1OC(C)=O ; CC(=O)O[C@H]1O[C@H](CCC2(N=[N+]=[N-])COC2)[C@@H](OC(C)=O)[C@H](OC(C)=O)[C@@H]1OC(C)=O ; CC(=O)O[C@H]1O[C@H](CCC2(N)COC2)[C@@H](OC(C)=O)[C@H](OC(C)=O)[C@@H]1OC(C)=O ; NC1(CC[C@H]2O[C@H](O)[C@@H](O)[C@@H](O)[C@@H]2O)COC1]

### Synthesis 2-36. Preparation of Structure 85

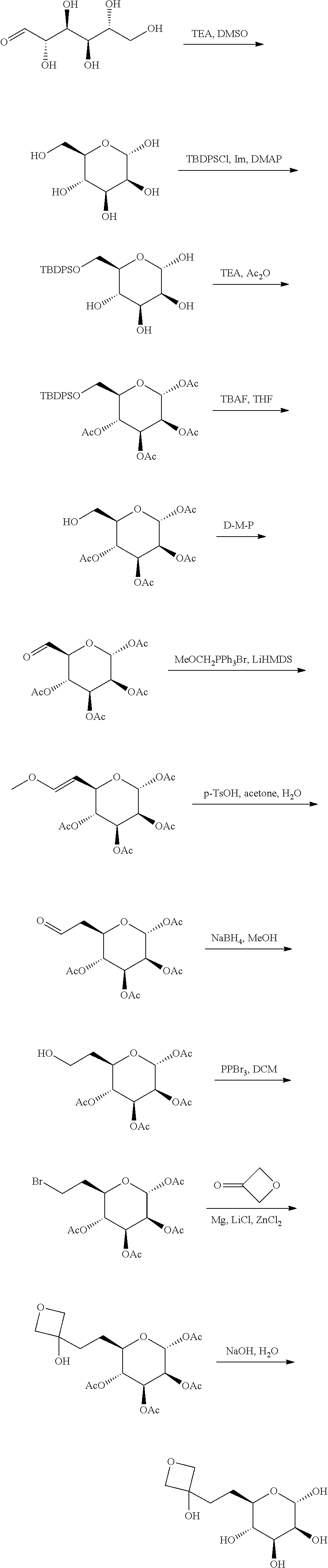

### Synthesis 2-37. Preparation of Structure 86, 87, 88

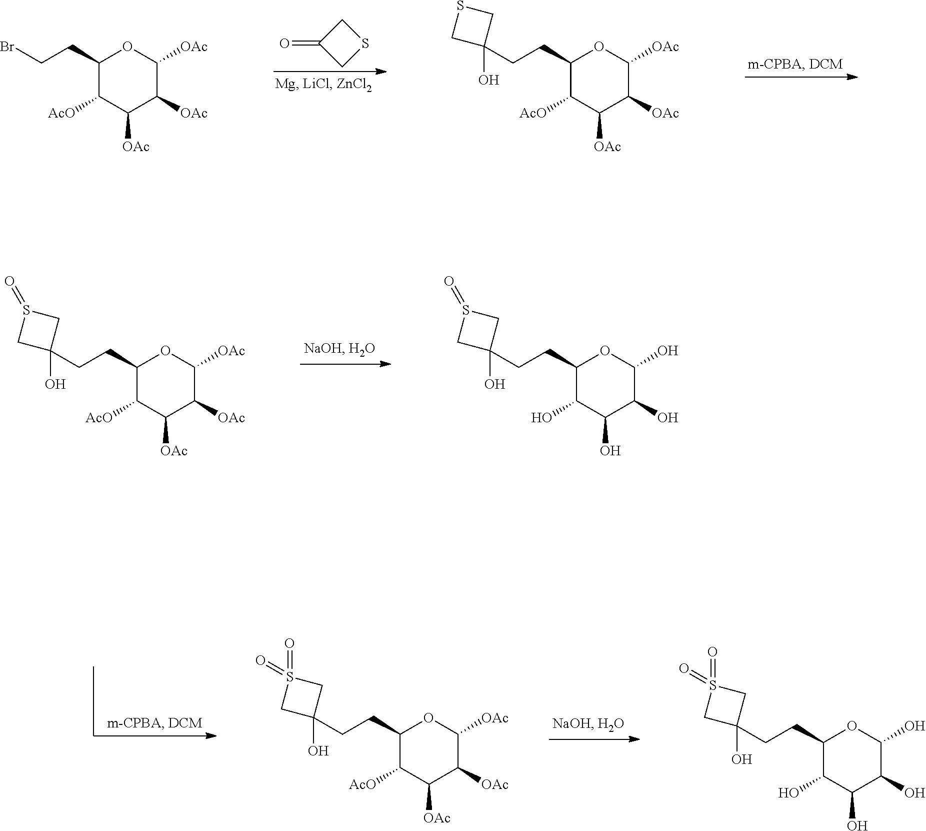

### Synthesis 2-38. Preparation of Structure 89

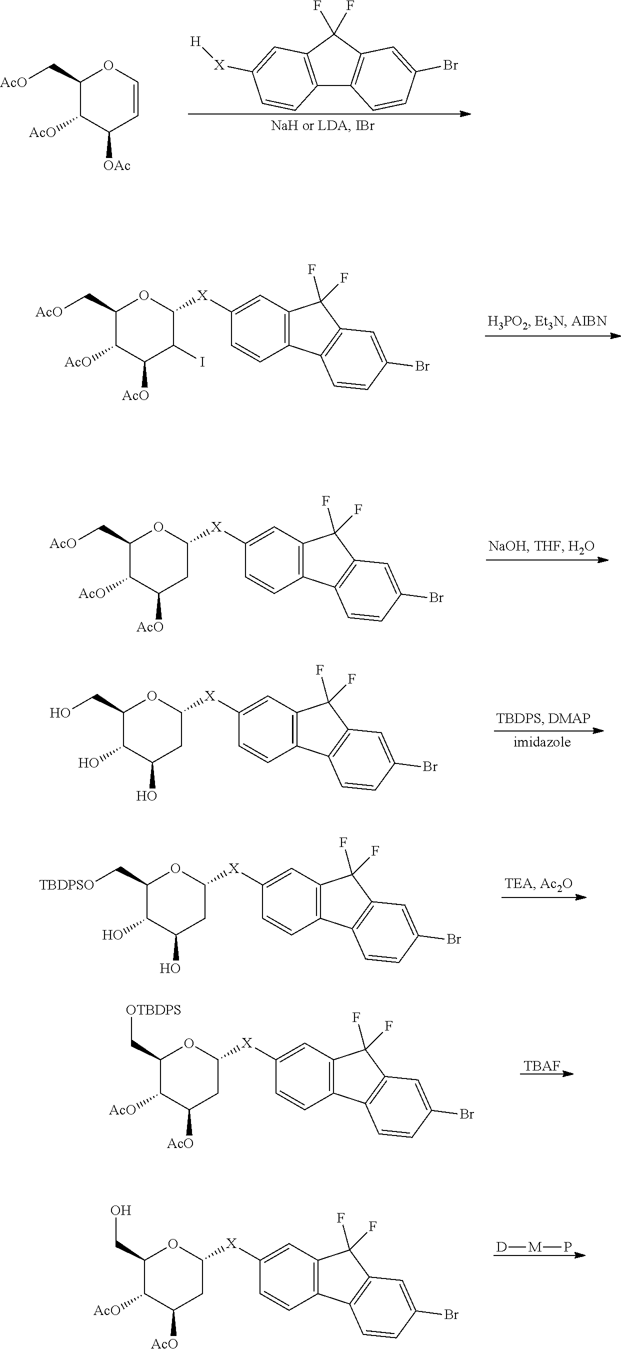

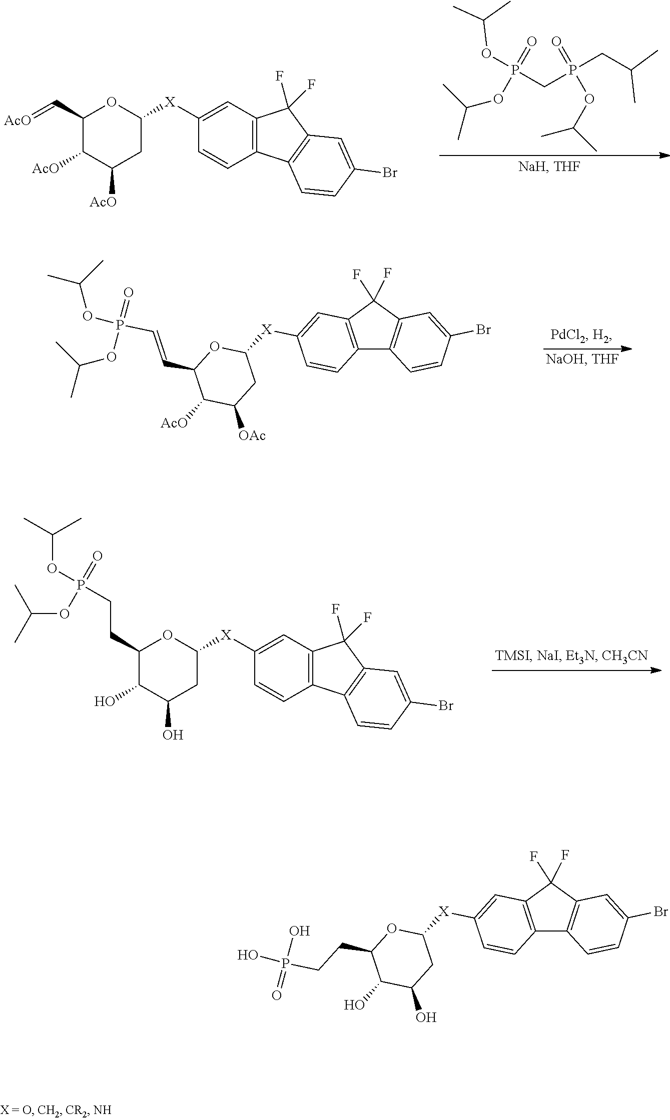

The following three compounds were synthesized according the above same route:

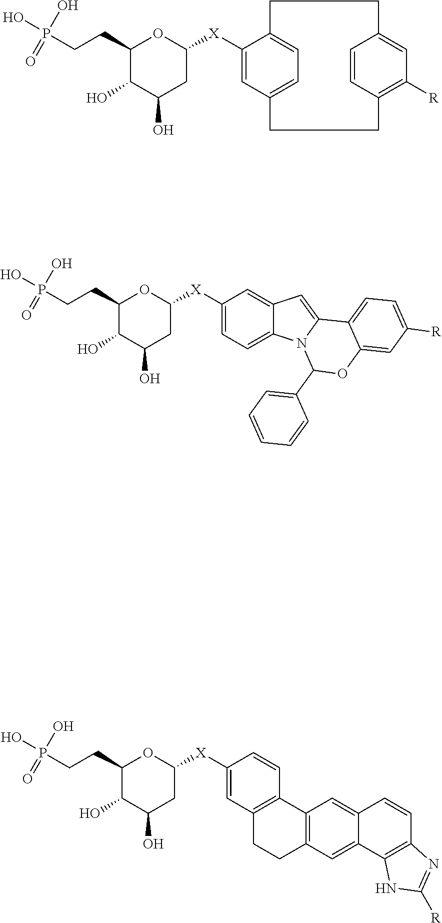

### Synthesis 2-39. Preparation of Structure 90, 91, 92

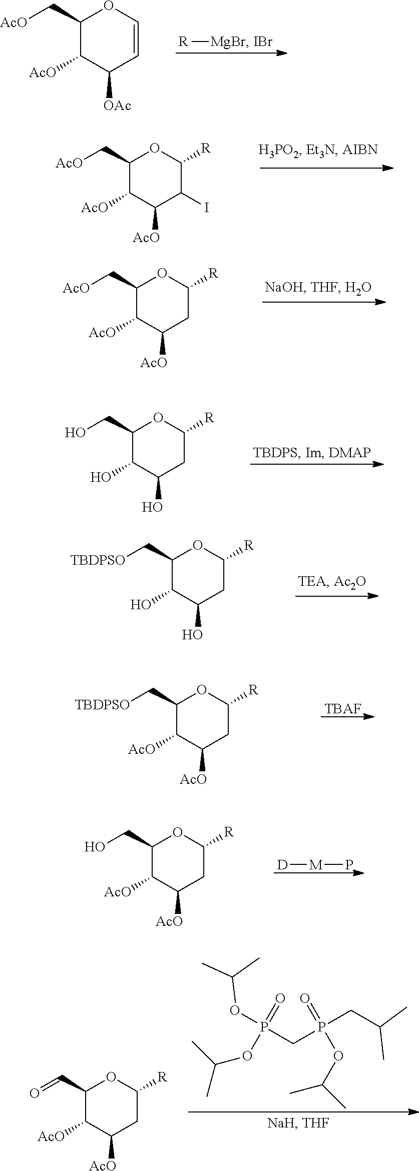

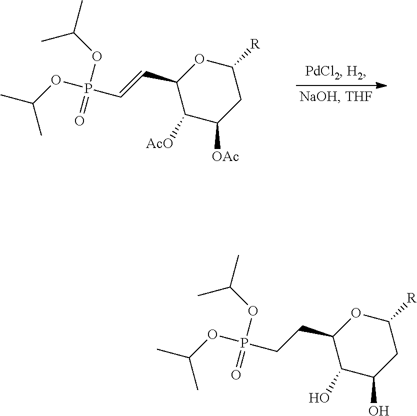

### Synthesis 2-40. Preparation of Structure 93, 170

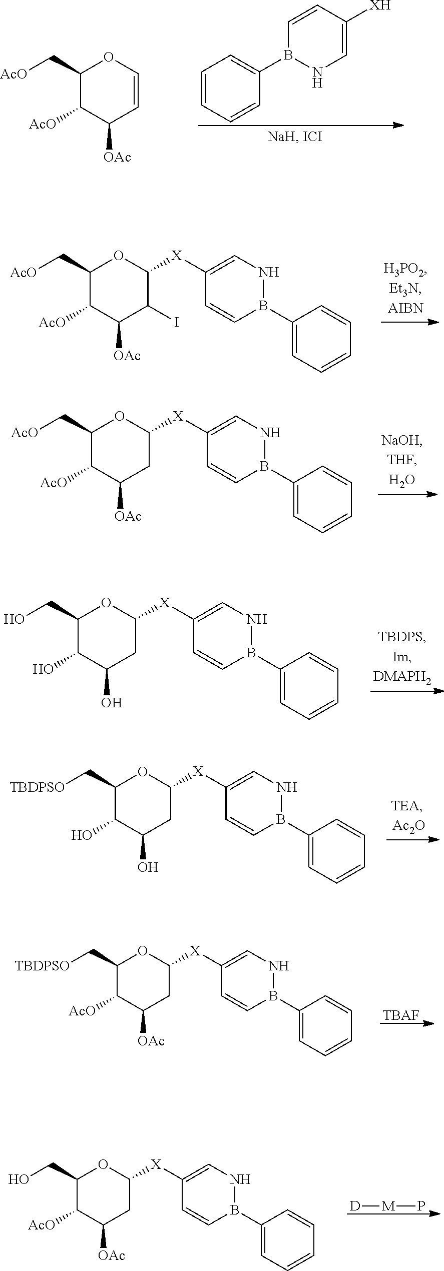

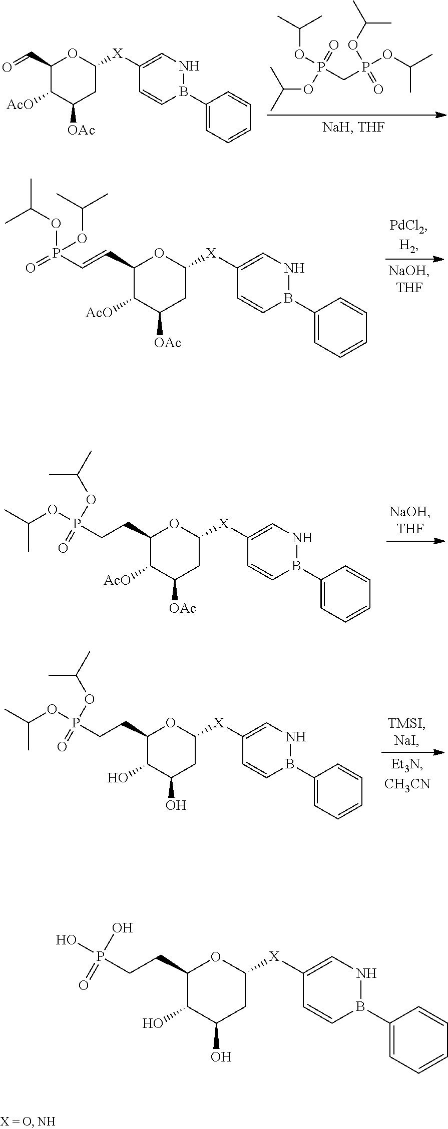

### Synthesis 2-41. Preparation of Structure 94

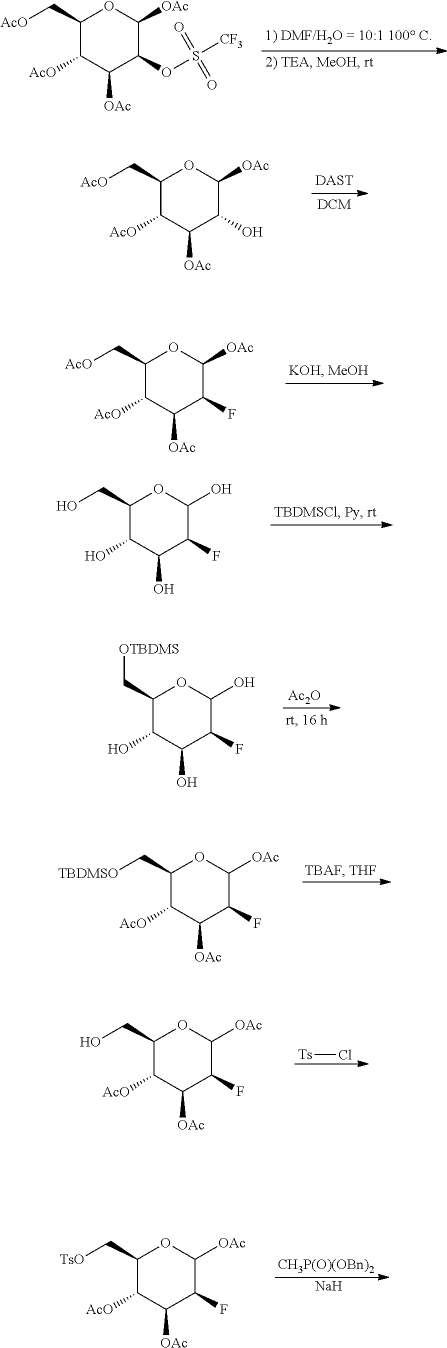

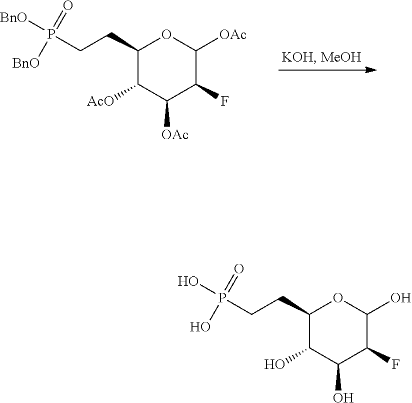 [SMILES: CC(=O)OC1O[C@H](CCP(=O)(OCc2ccccc2)OCc2ccccc2)[C@@H](OC(C)=O)[C@H](OC(C)=O)[C@@H]1F ; O=P(O)(O)CC[C@H]1OC(O)[C@@H](F)[C@@H](O)[C@@H]1O]

### Synthesis 2-42. Preparation of Structure 95

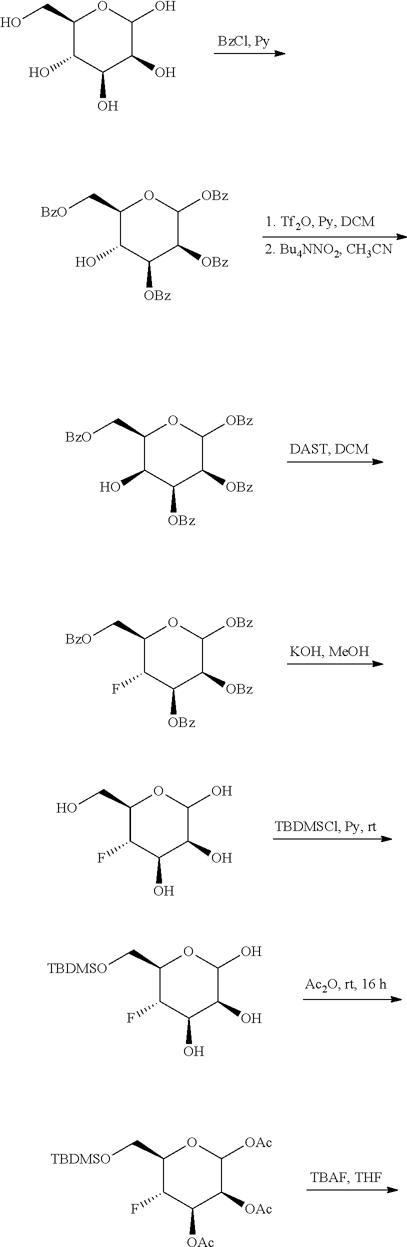

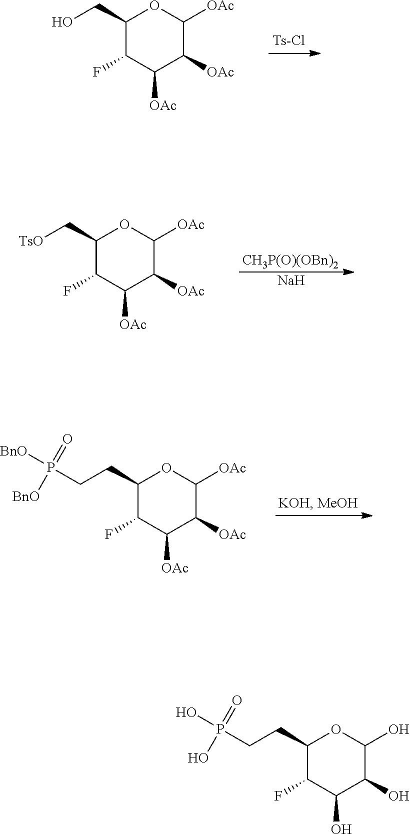

### Synthesis 2-43. Preparation of Structure 96

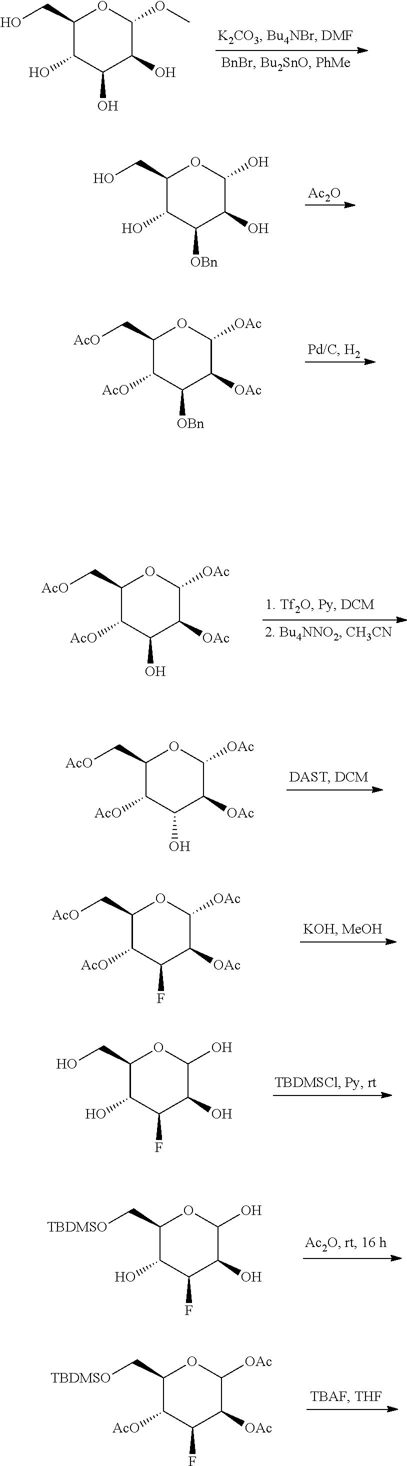

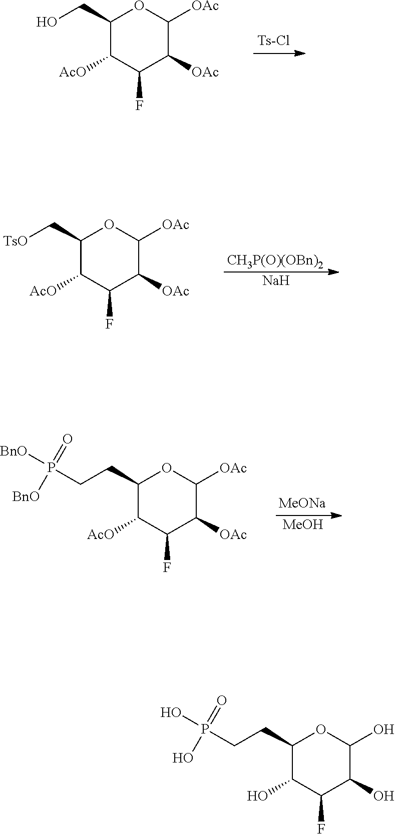

### Synthesis 2-44. Preparation of Structure 97

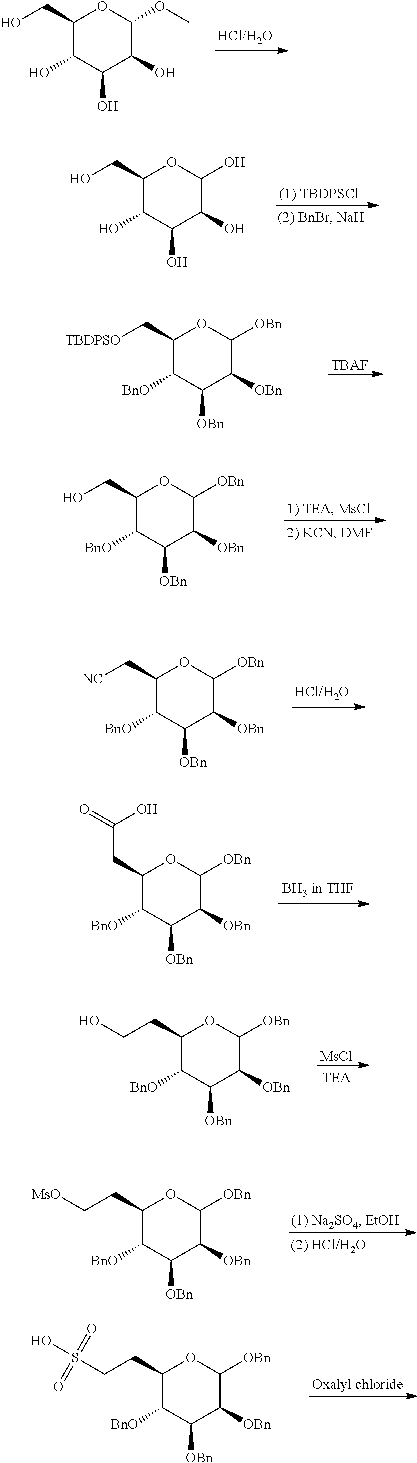

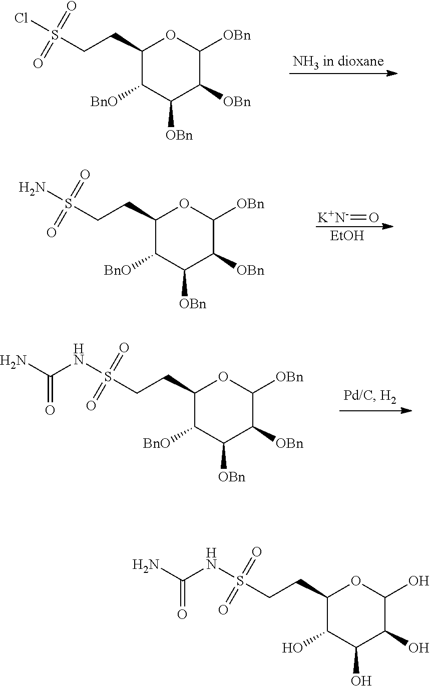

### Synthesis 2-45. Preparation of Structure 98

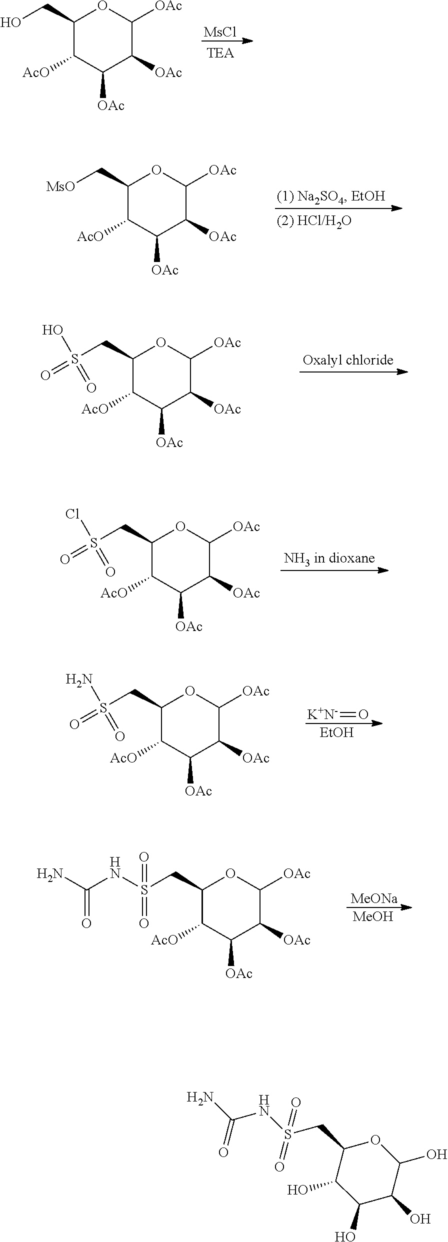

### Synthesis 2-46. Preparation of Structure 99

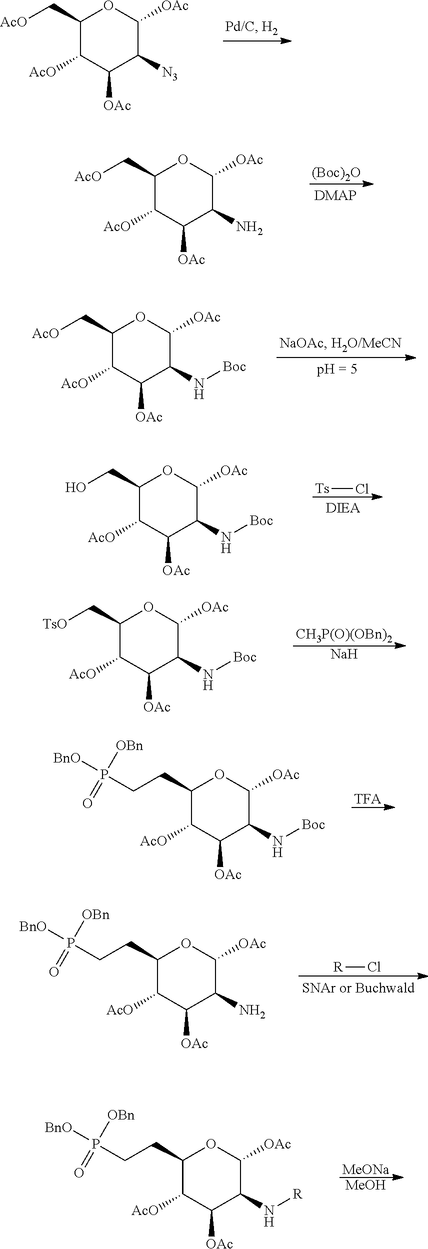

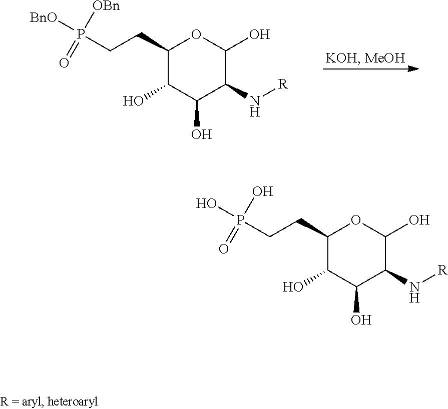

### Synthesis 2-47. Preparation of Structure 100

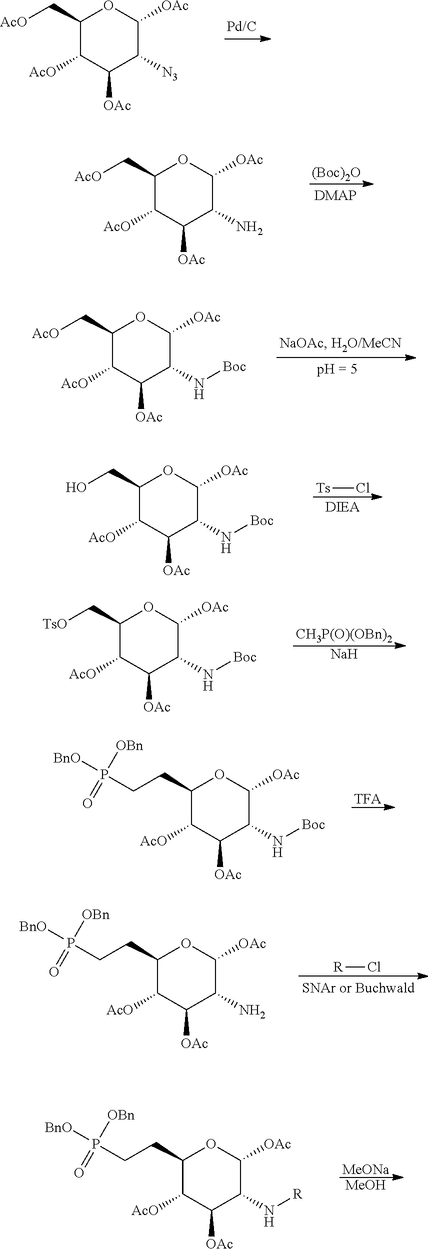

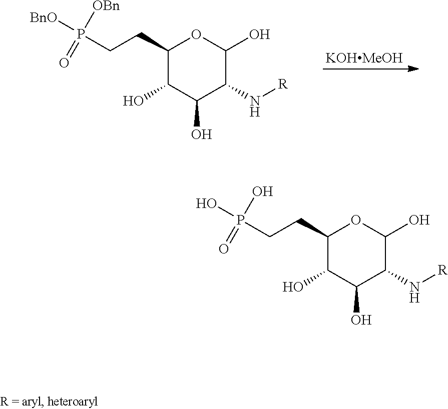

### Synthesis 2-48. Preparation of Structure 101

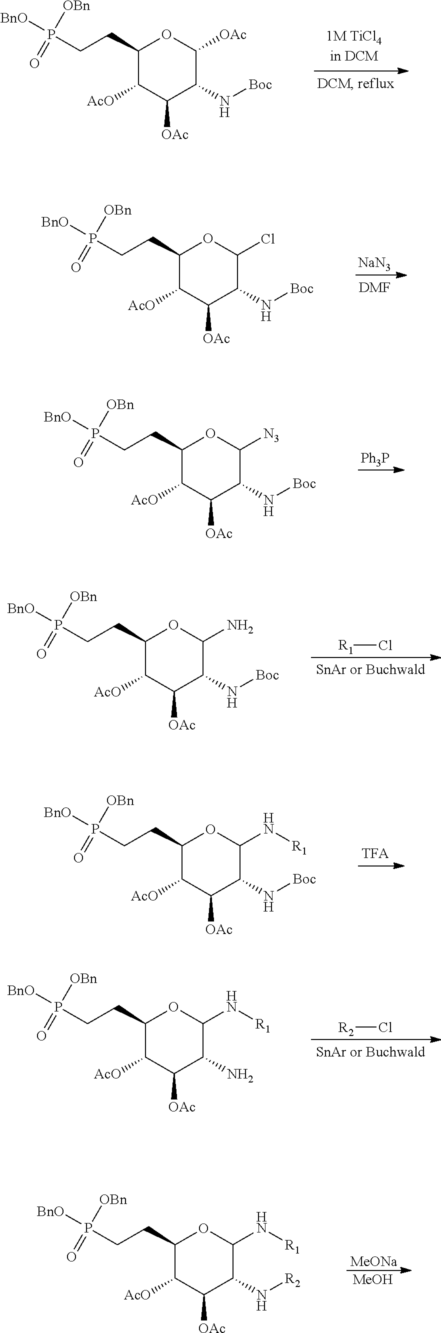

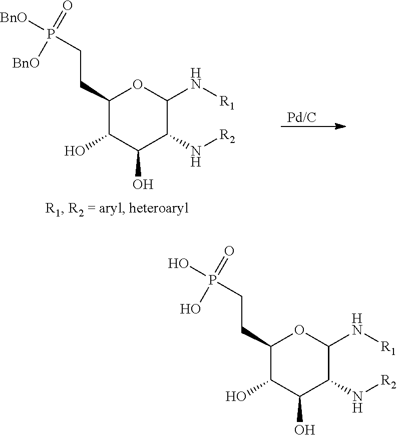

### Synthesis 2-49. Preparation of Structure 102

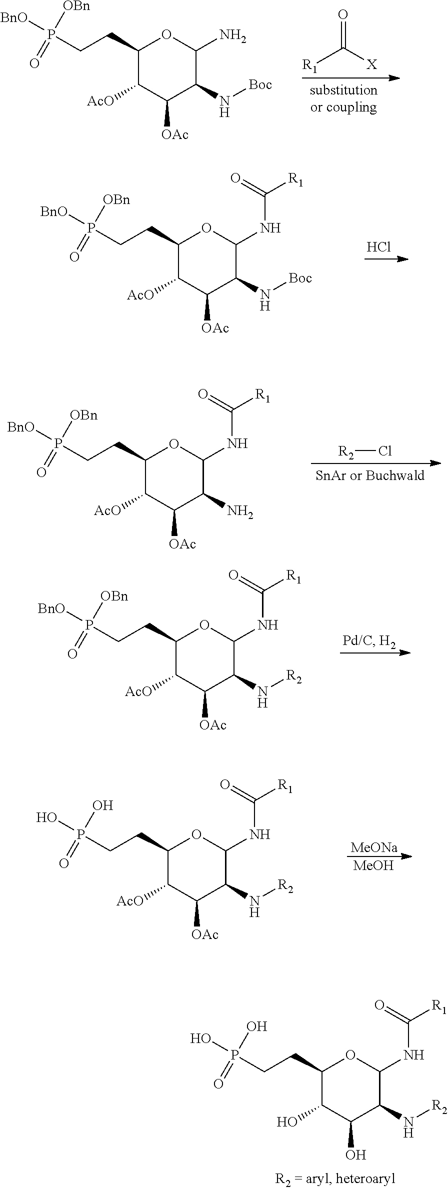

### Synthesis 2-50. Preparation of Structure 105

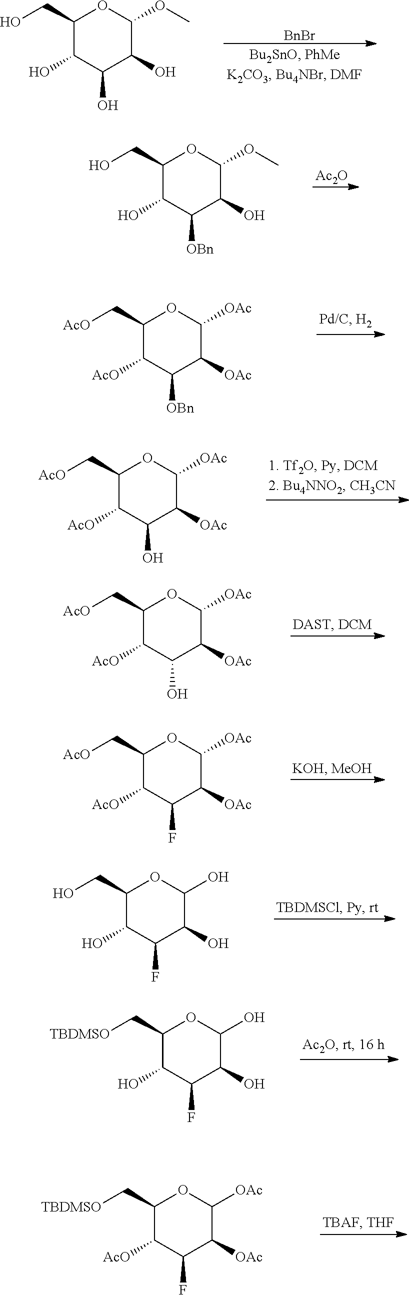

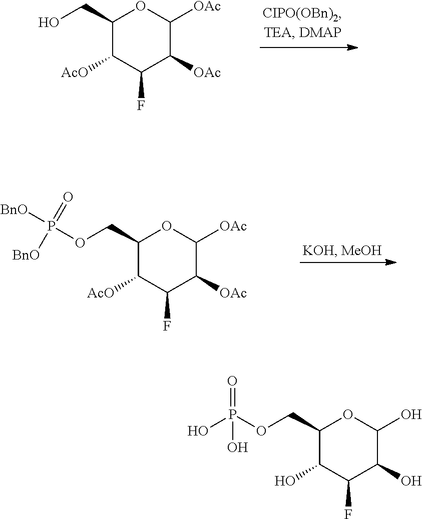 [SMILES: CC(=O)OC1O[C@H](CO)[C@@H](OC(C)=O)[C@H](F)[C@@H]1OC(C)=O ; CC(=O)OC1O[C@H](COP(=O)(OCc2ccccc2)OCc2ccccc2)[C@@H](OC(C)=O)[C@H](F)[C@@H]1OC(C)=O ; O=P(O)(O)OC[C@H]1OC(O)[C@@H](O)[C@@H](F)[C@@H]1O]

### Synthesis 2-51. Preparation of Structure 112

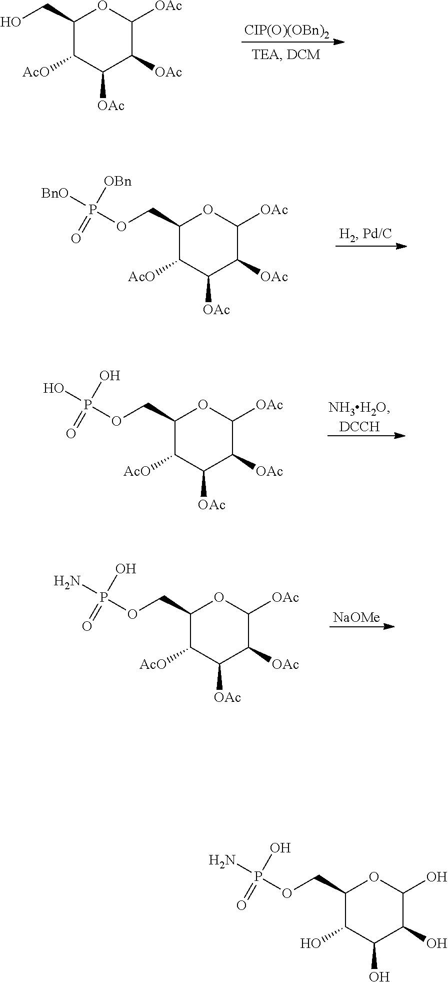

### Synthesis 2-52. Preparation of Structure 115

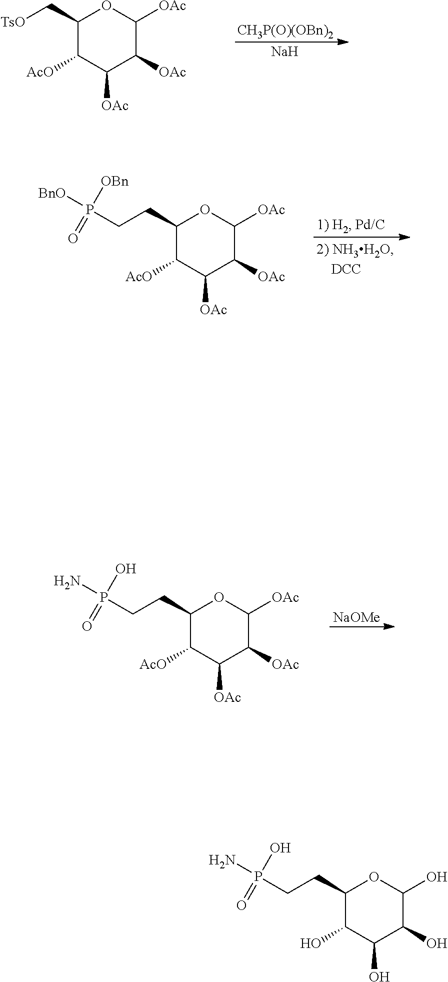

### Synthesis 2-53. Preparation of Structure 116

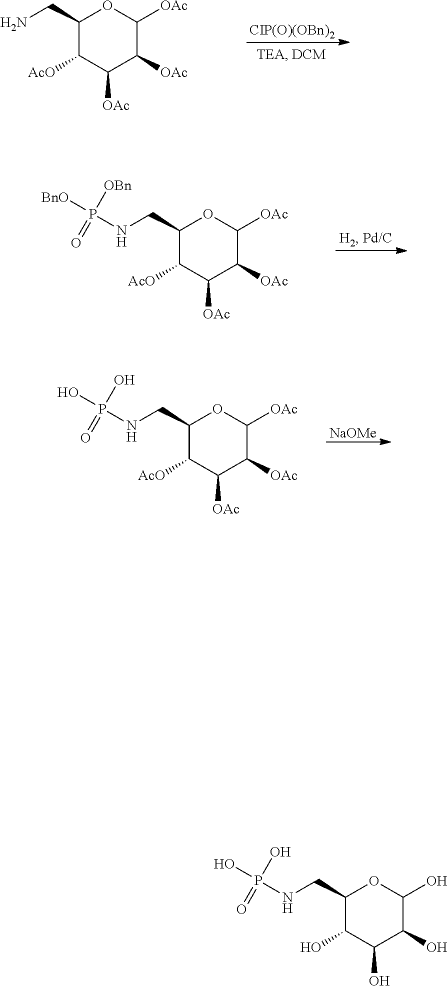

### Synthesis 2-54. Preparation of Structure 117

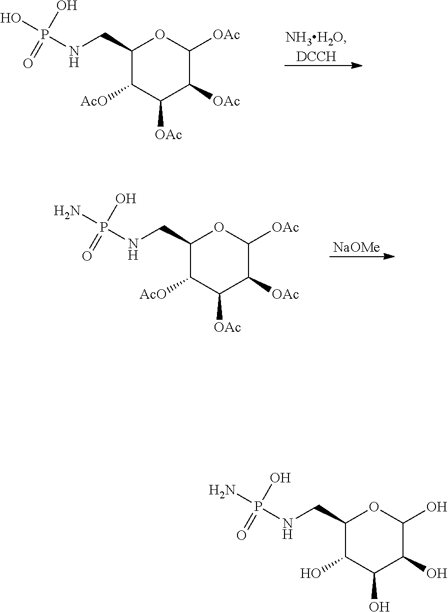

### Synthesis 2-55. Preparation of Structure 118

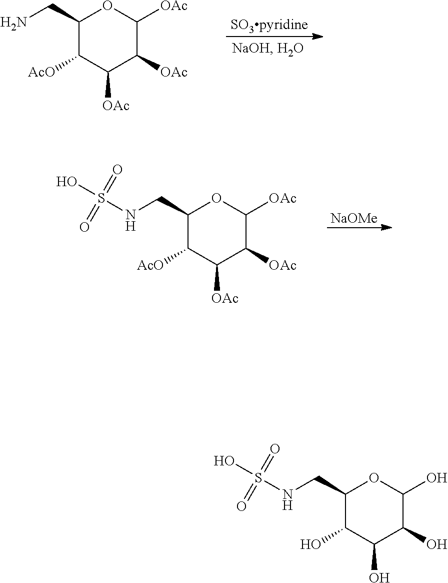 [SMILES: CC(=O)OC1O[C@H](CN)[C@@H](OC(C)=O)[C@H](OC(C)=O)[C@@H]1OC(C)=O ; "SO3·pyridine" ; CC(=O)OC1O[C@H](CNS(=O)(=O)O)[C@@H](OC(C)=O)[C@H](OC(C)=O)[C@@H]1OC(C)=O ; O=S(=O)(O)NC[C@H]1OC(O)[C@@H](O)[C@@H](O)[C@@H]1O ; "NaOMe"]

### Synthesis 2-56. Preparation of Structure 119

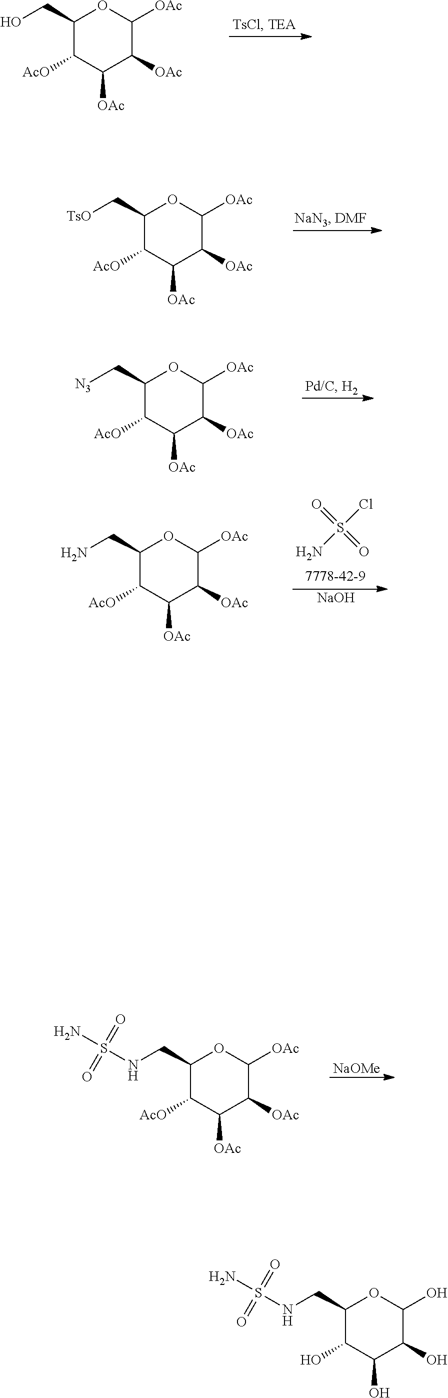

### Synthesis 2-57. Preparation of Structure 122

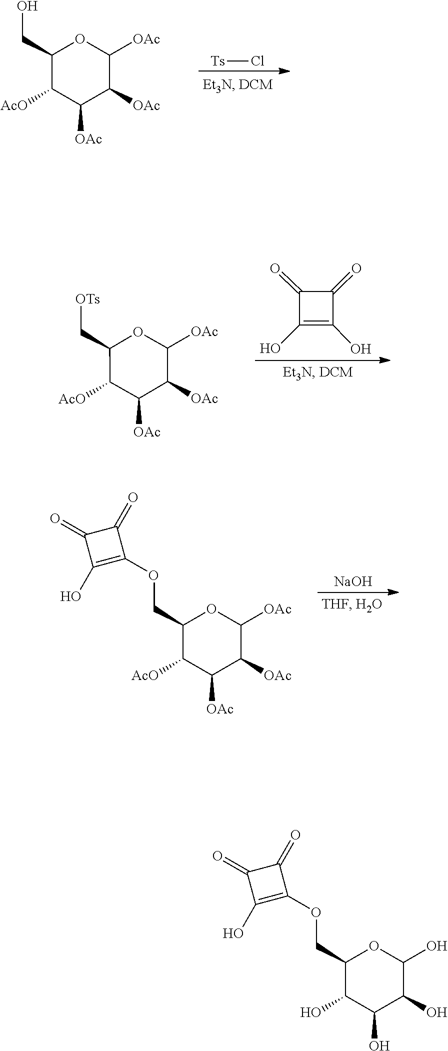

### Synthesis 2-58. Preparation of Structure 123

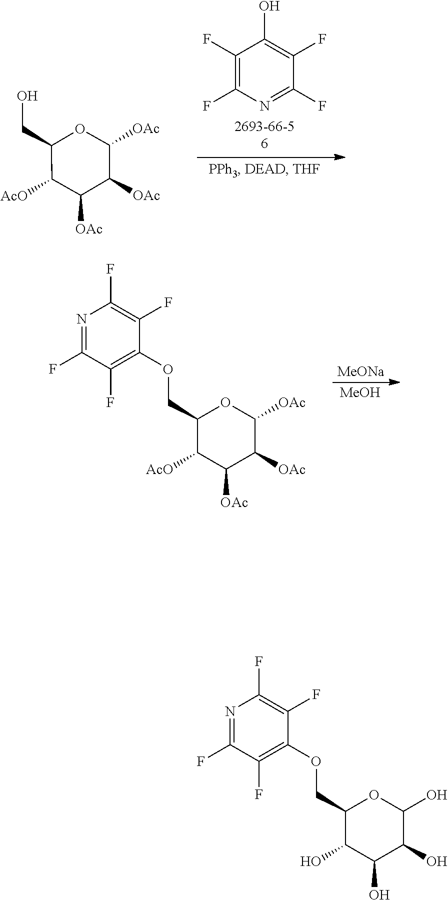 [SMILES: "MeONa" ; CC(=O)O[C@H]1O[C@H](CO)[C@@H](OC(C)=O)[C@H](OC(C)=O)[C@@H]1OC(C)=O ; "MeOH" ; Oc1c(F)c(F)nc(F)c1F ; CC(=O)O[C@H]1O[C@H](COc2c(F)c(F)nc(F)c2F)[C@@H](OC(C)=O)[C@H](OC(C)=O)[C@@H]1OC(C)=O ; OC1O[C@H](COc2c(F)c(F)nc(F)c2F)[C@@H](O)[C@H](O)[C@@H]1O]

### Synthesis 2-59. Preparation of Structures 127-133

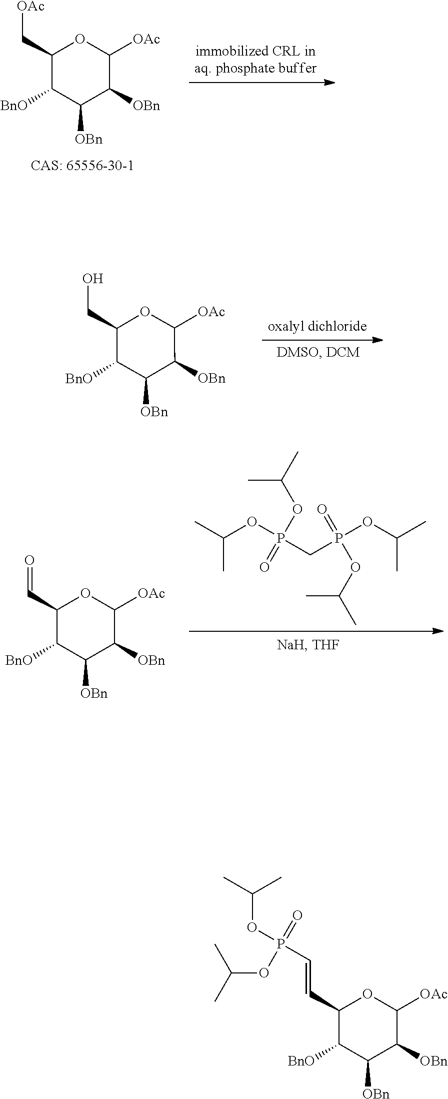 [SMILES: CC(=O)OC[C@H]1OC(OC(C)=O)[C@@H](OCc2ccccc2)[C@@H](OCc2ccccc2)[C@@H]1OCc1ccccc1 ; CC(=O)OC1O[C@H](CO)[C@@H](OCc2ccccc2)[C@H](OCc2ccccc2)[C@@H]1OCc1ccccc1 ; CC(=O)OC1O[C@H](C=O)[C@@H](OCc2ccccc2)[C@H](OCc2ccccc2)[C@@H]1OCc1ccccc1 ; CC(C)OP(=O)(CP(=O)(OC(C)C)OC(C)C)OC(C)C ; CC(=O)OC1O[C@H](/C=C/P(=O)(OC(C)C)OC(C)C)[C@@H](OCc2ccccc2)[C@H](OCc2ccccc2)[C@@H]1OCc1ccccc1]

### Synthesis 2-60. Preparation of Structures 134-137

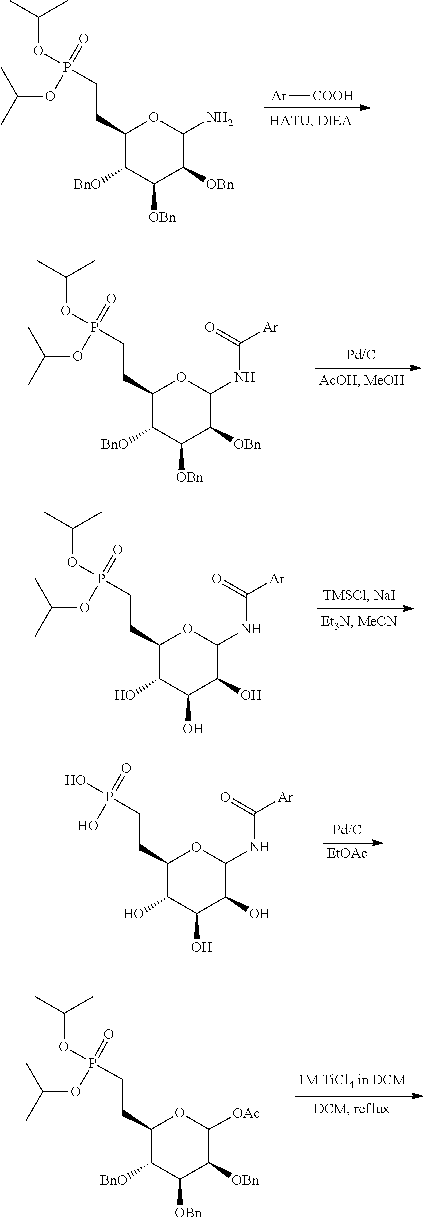

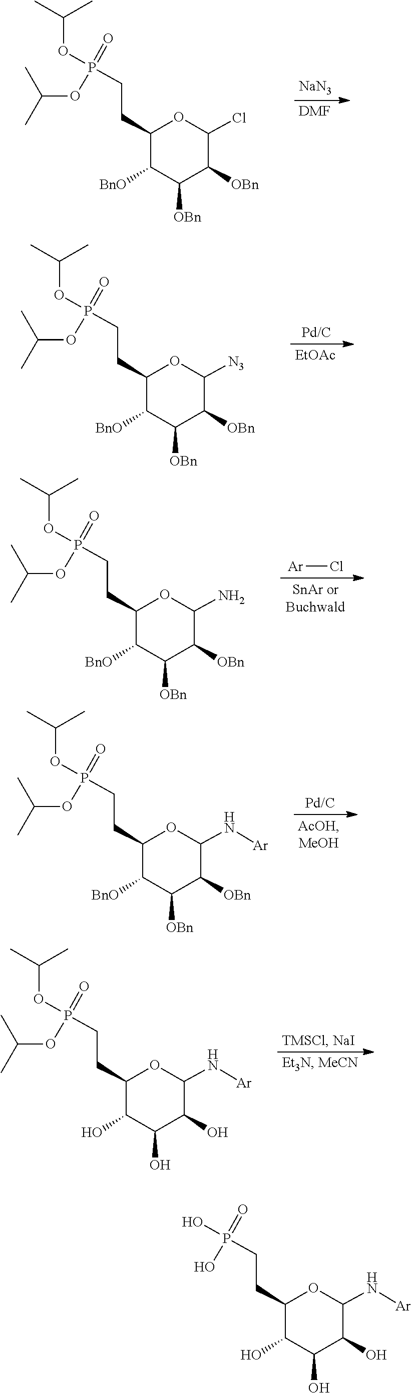

### Synthesis 2-61. Preparation of Structure 138

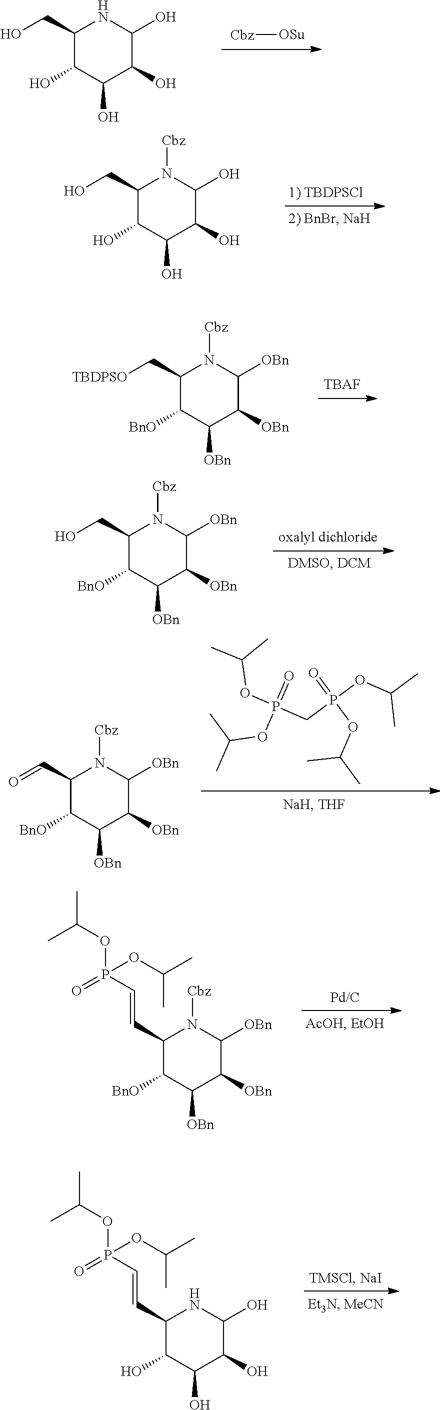

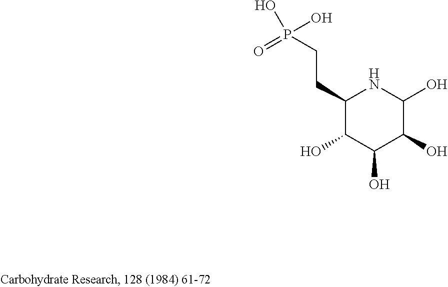 [SMILES: O=P(O)(O)CC[C@H]1NC(O)[C@@H](O)[C@@H](O)[C@@H]1O]

### Synthesis 2-62. Preparation of Structures 139

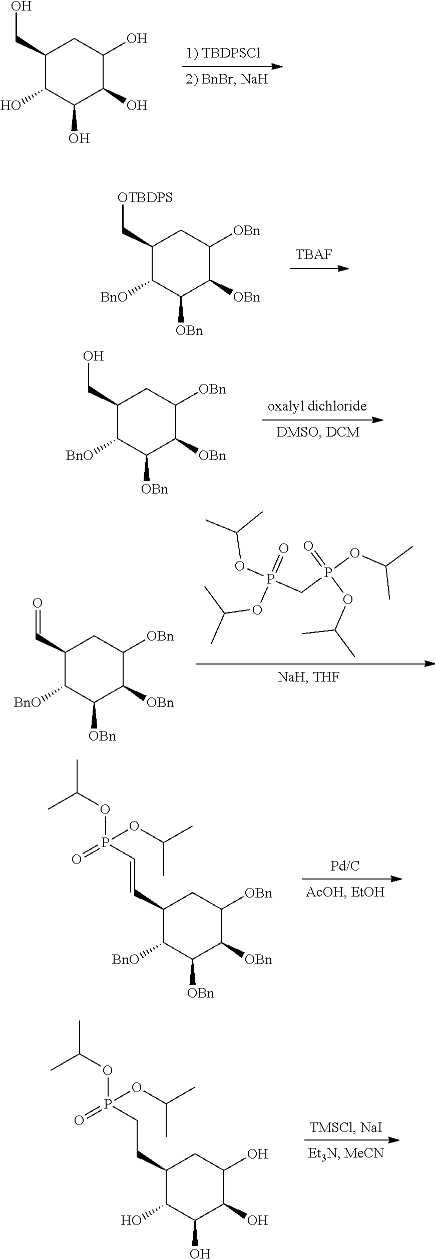

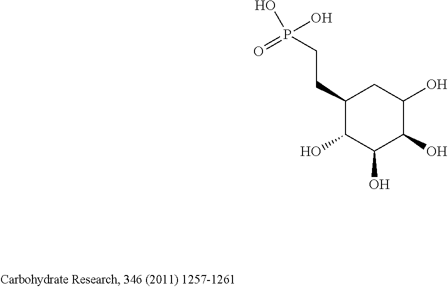 [SMILES: O=P(O)(O)CC[C@H]1CC(O)[C@@H](O)[C@@H](O)[C@@H]1O]

### Synthesis 2-63. Preparation of Structures 140-157

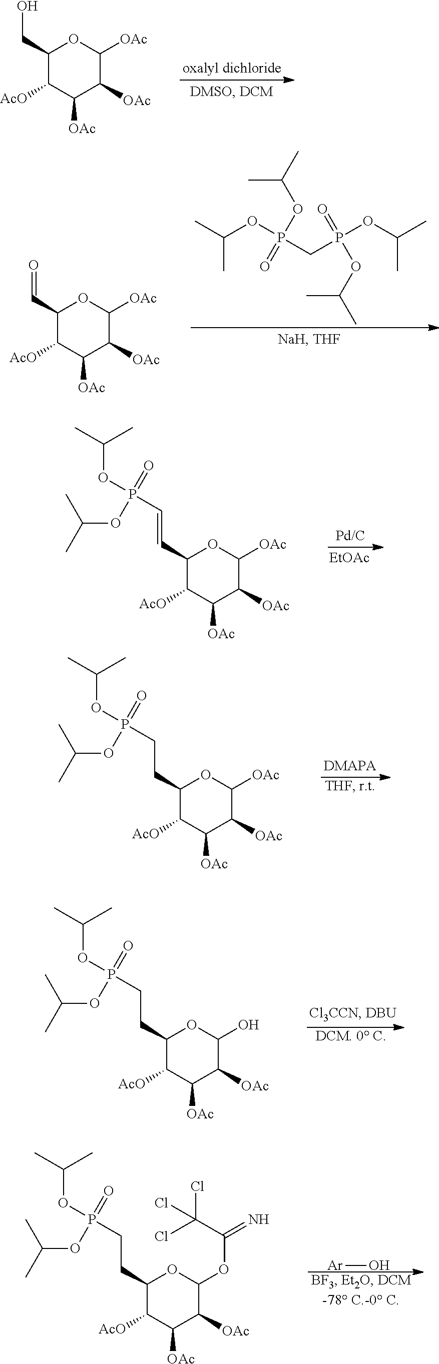

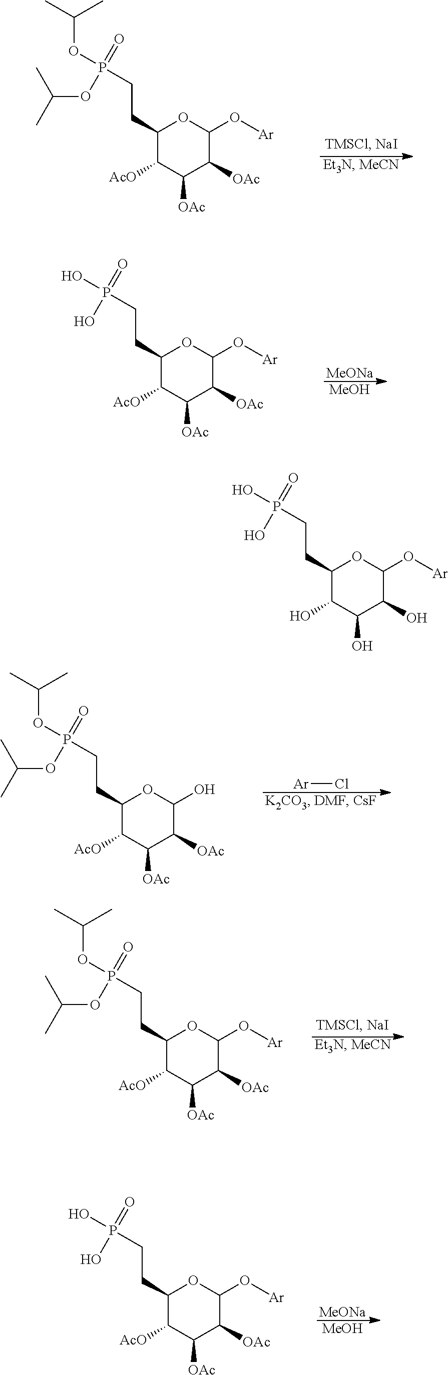

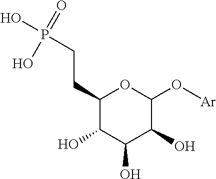

### Synthesis 2-64

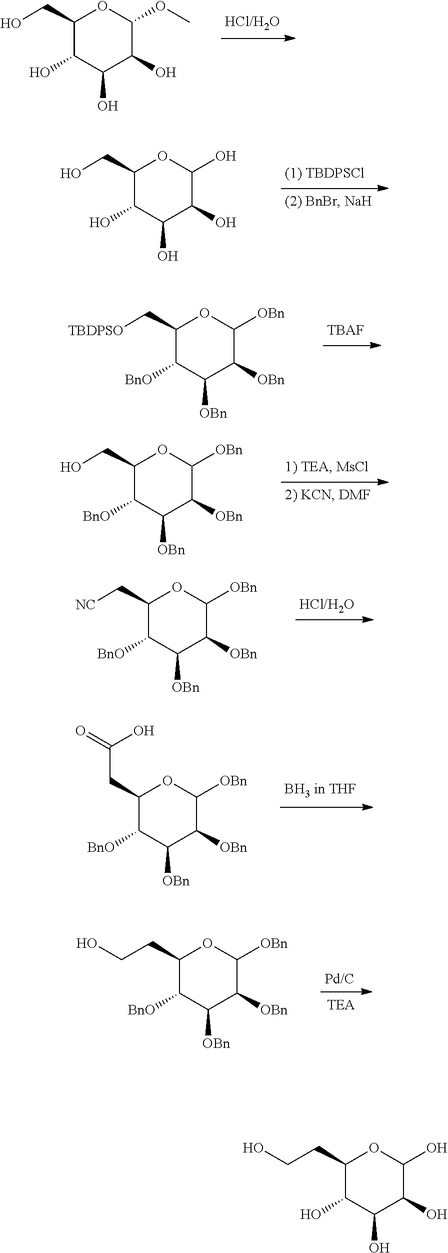

### Synthesis 2-65

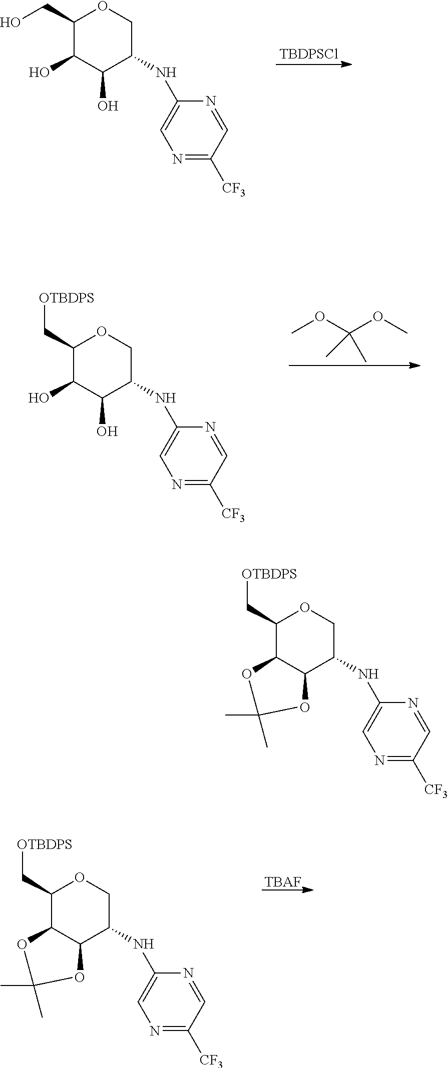

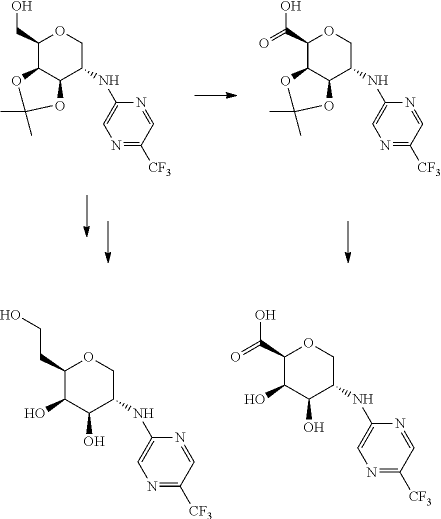 [SMILES: CC1(C)O[C@@H]2[C@H](O1)[C@@H](Nc1cnc(C(F)(F)F)cn1)CO[C@@H]2CO ; CC1(C)O[C@@H]2[C@H](O1)[C@@H](Nc1cnc(C(F)(F)F)cn1)CO[C@@H]2C(=O)O ; OCC[C@H]1OC[C@H](Nc2cnc(C(F)(F)F)cn2)[C@@H](O)[C@H]1O ; O=C(O)[C@H]1OC[C@H](Nc2cnc(C(F)(F)F)cn2)[C@@H](O)[C@H]1O]

### Synthesis 2-66

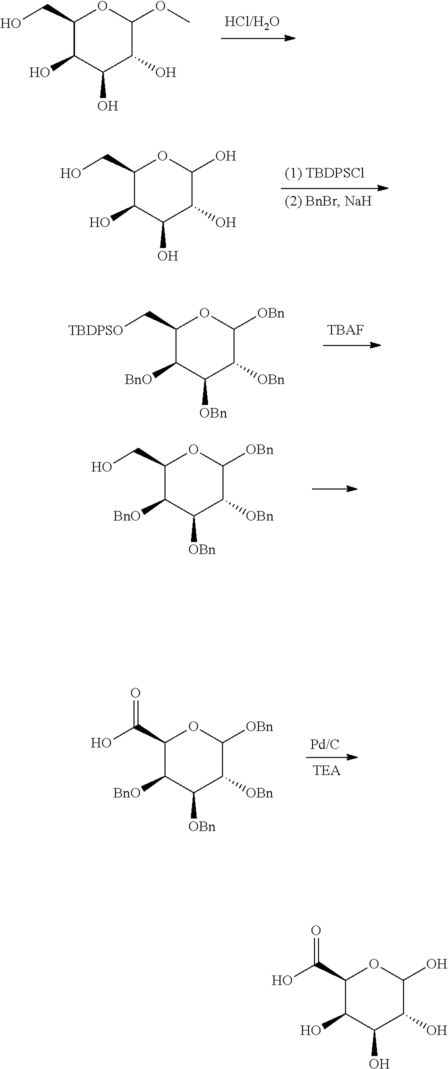

### Example 3. Synthesis and Installation of Linkers

Linker^{B} can be synthesized from any chemical moiety containing at least two reactive sites for bond formation. For example, a compound of the present invention containing Linker^{B} can be synthesized from:

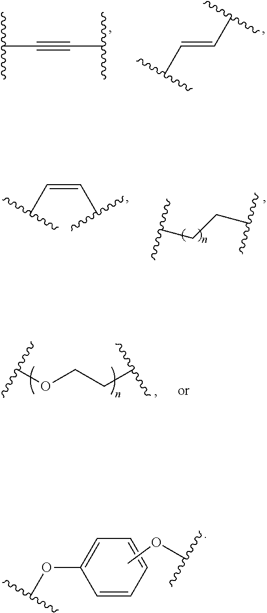

Linker^{C} can be synthesized from any chemical moiety containing at least three reactive sites for bond formation. For example, a compound of the present invention containing Linker^{C} can be synthesized from:

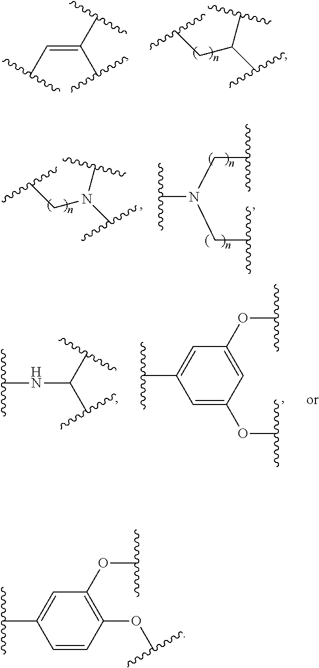

Linker^{D} can be synthesized from any chemical moiety containing at least three reactive sites for bond formation. For example, a compound of the present invention containing Linker^{D} can be synthesized from:

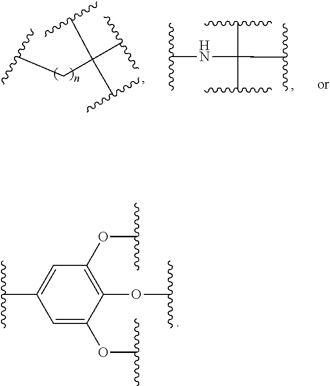

### Synthesis 3-1. Attachment of Triazole-Containing Alkyl and Polyethylene Glycol Linkers

### For Linear Alkyl:

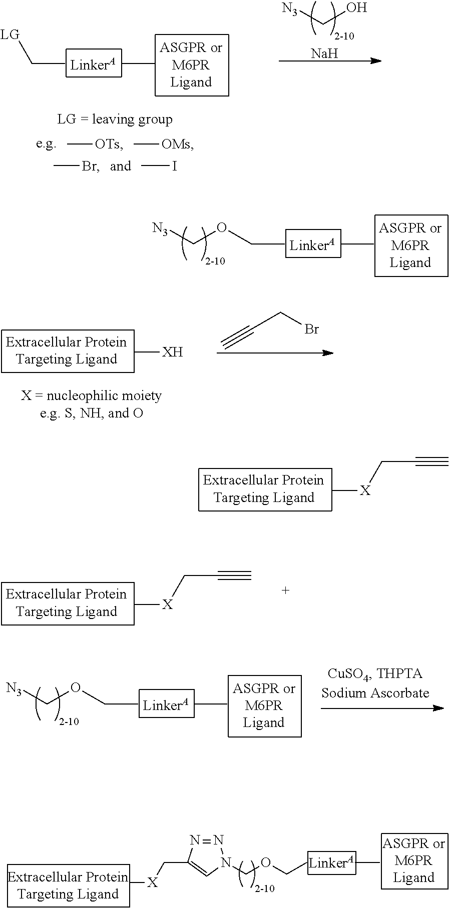

### For Polyethylene Glycol:

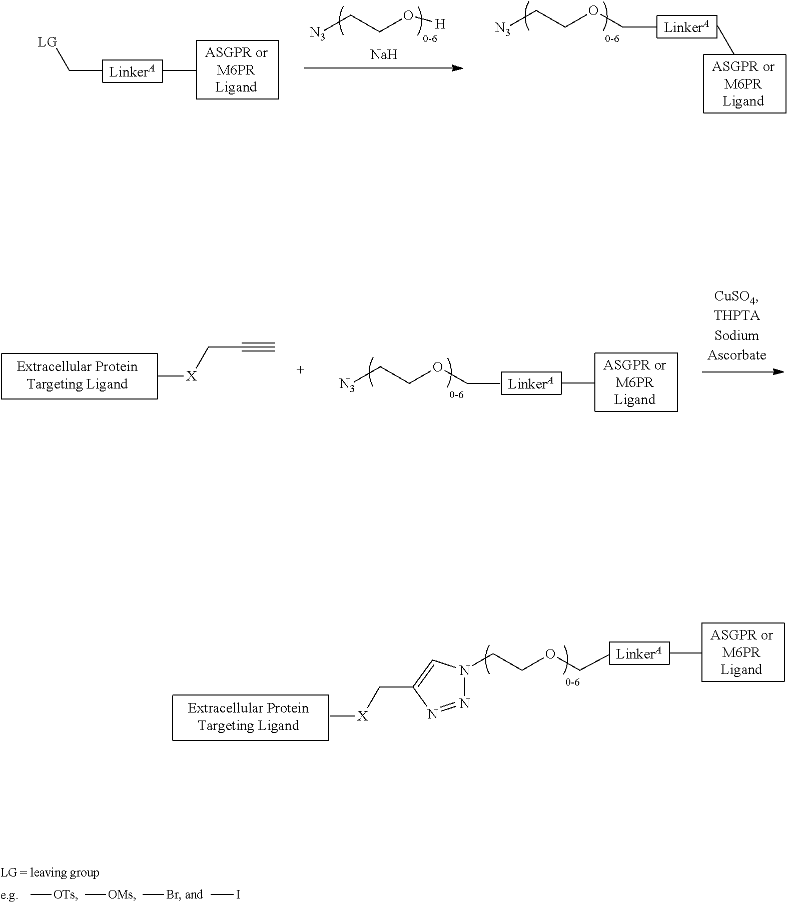

### Synthesis 3-2. Attachment of 1-(3,4-dihydroxybenzyl)-1H-pyrrole-2,5-dione Linker

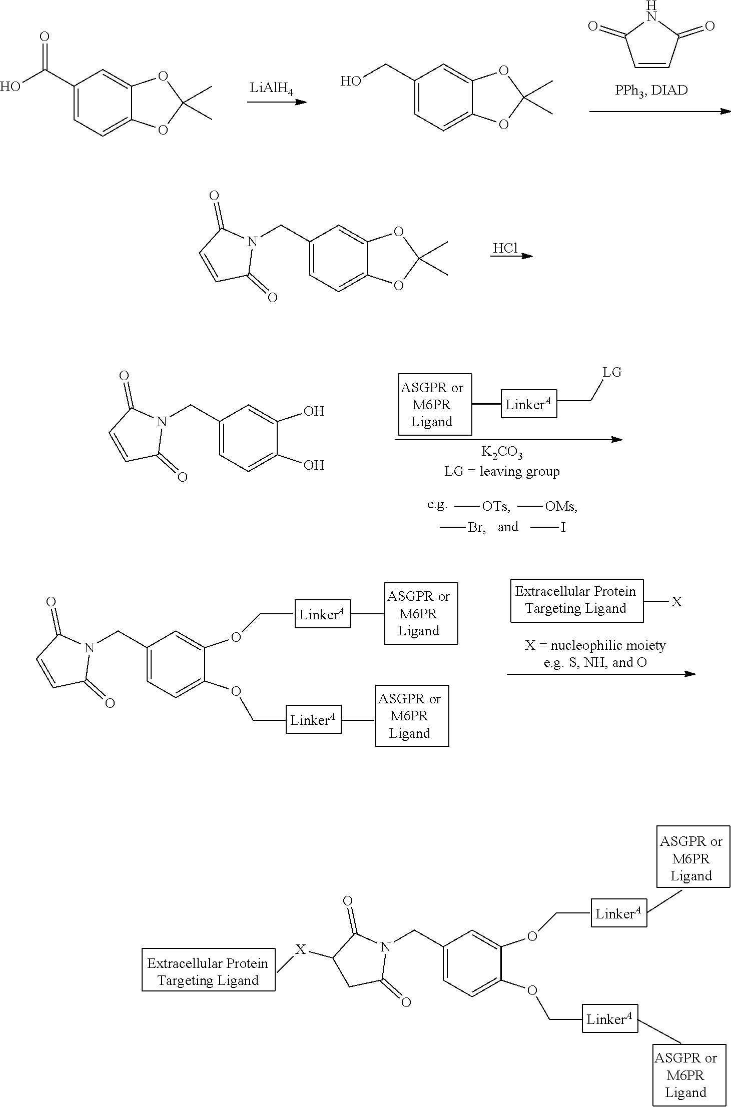

### Synthesis 3-3. Attachment of 1-(carboxymethyl)-5-hydroxy-1H-indole-3-carboxylic acid Linker

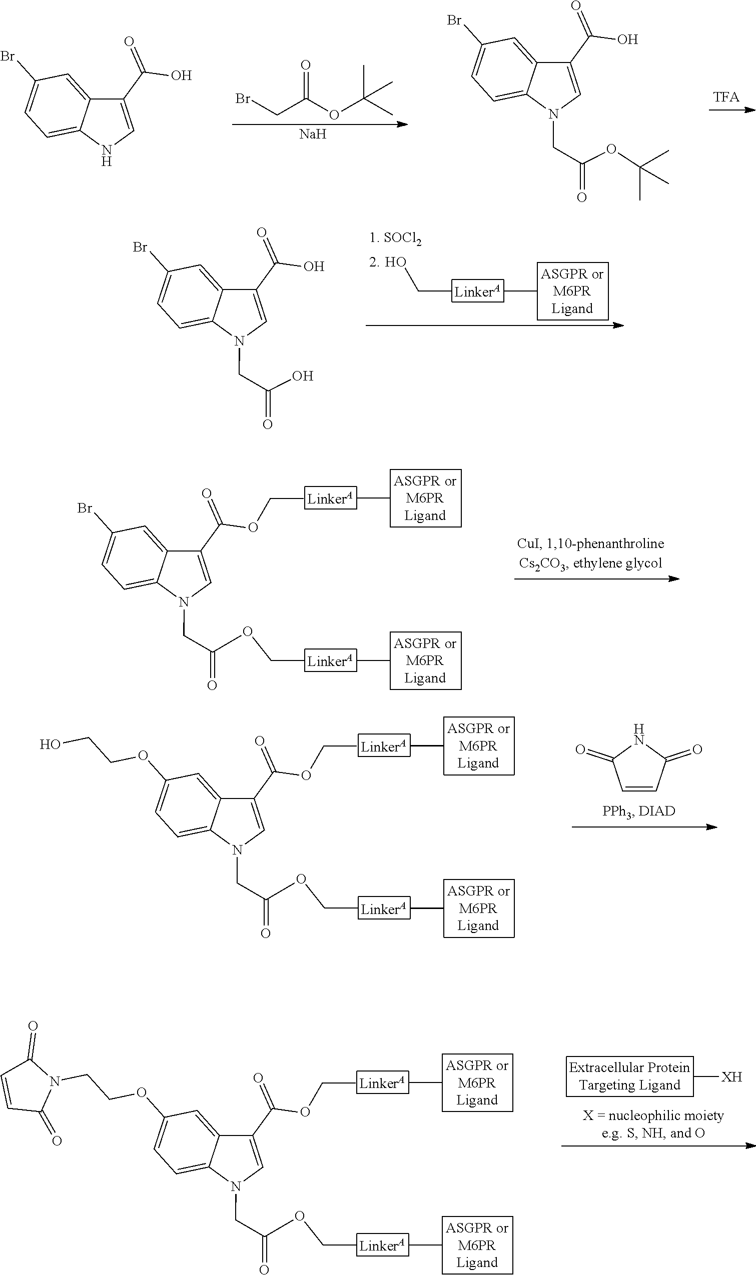

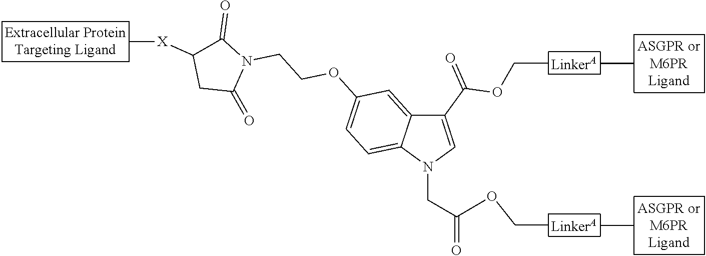

### Synthesis 3-4. Attachment of pyrrolidine-3,4-diamine Linker

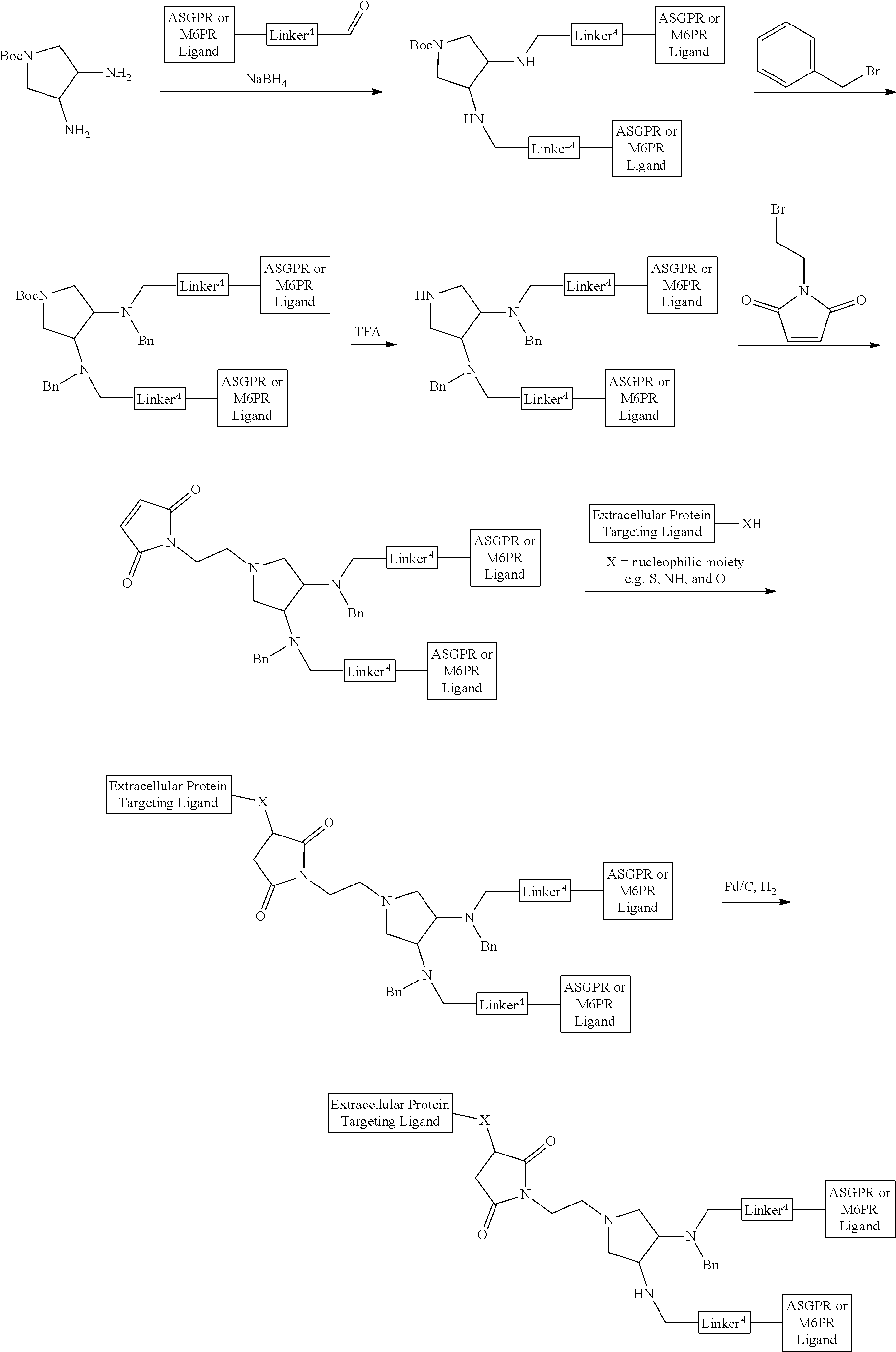

### Synthesis 3-5. Attachment of Cyclobutane-1,3-Diol-Containing Alkyl and Polyethylene Glycol Linkers

### For Linear Alkyl:

### Linker^{C}:

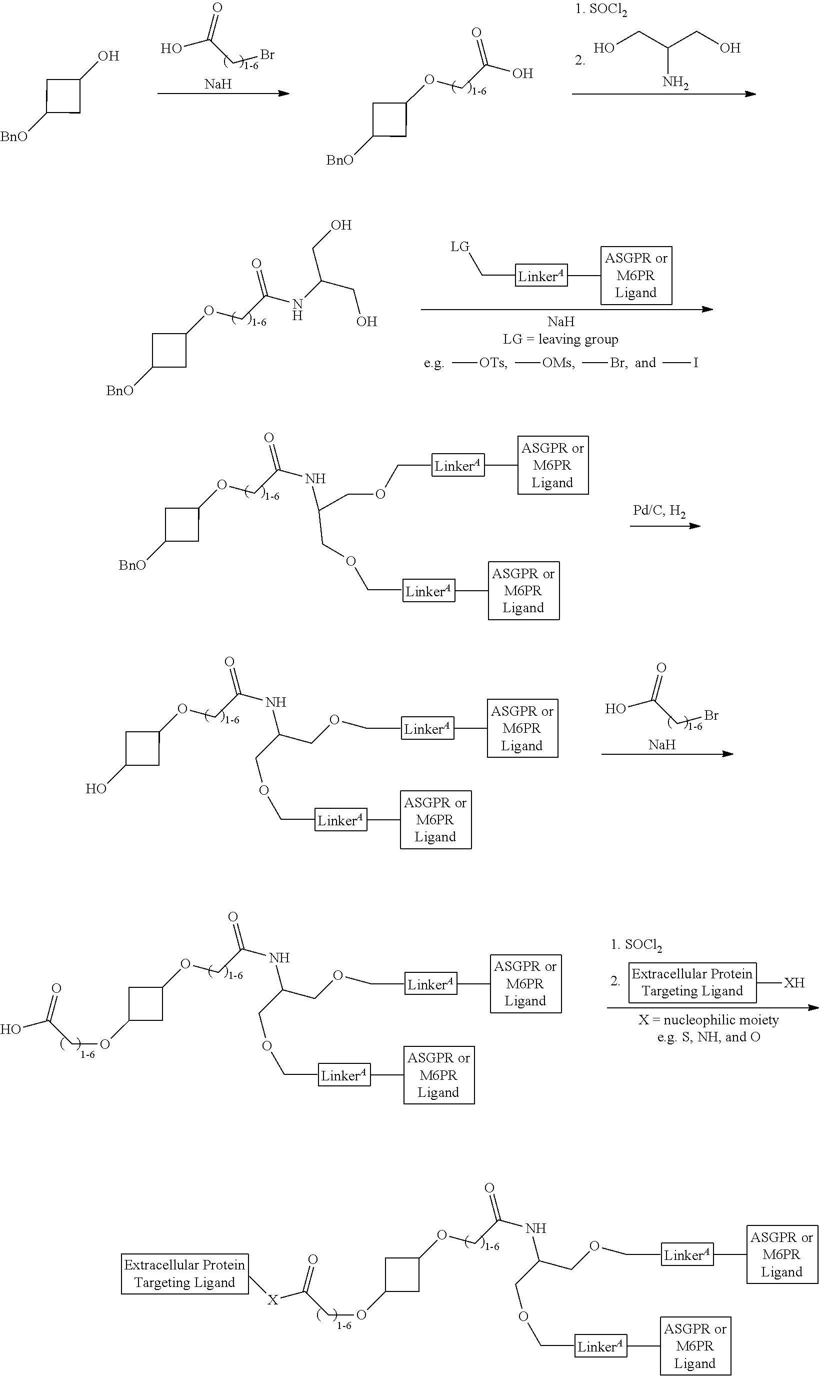

### Linker^{D}:

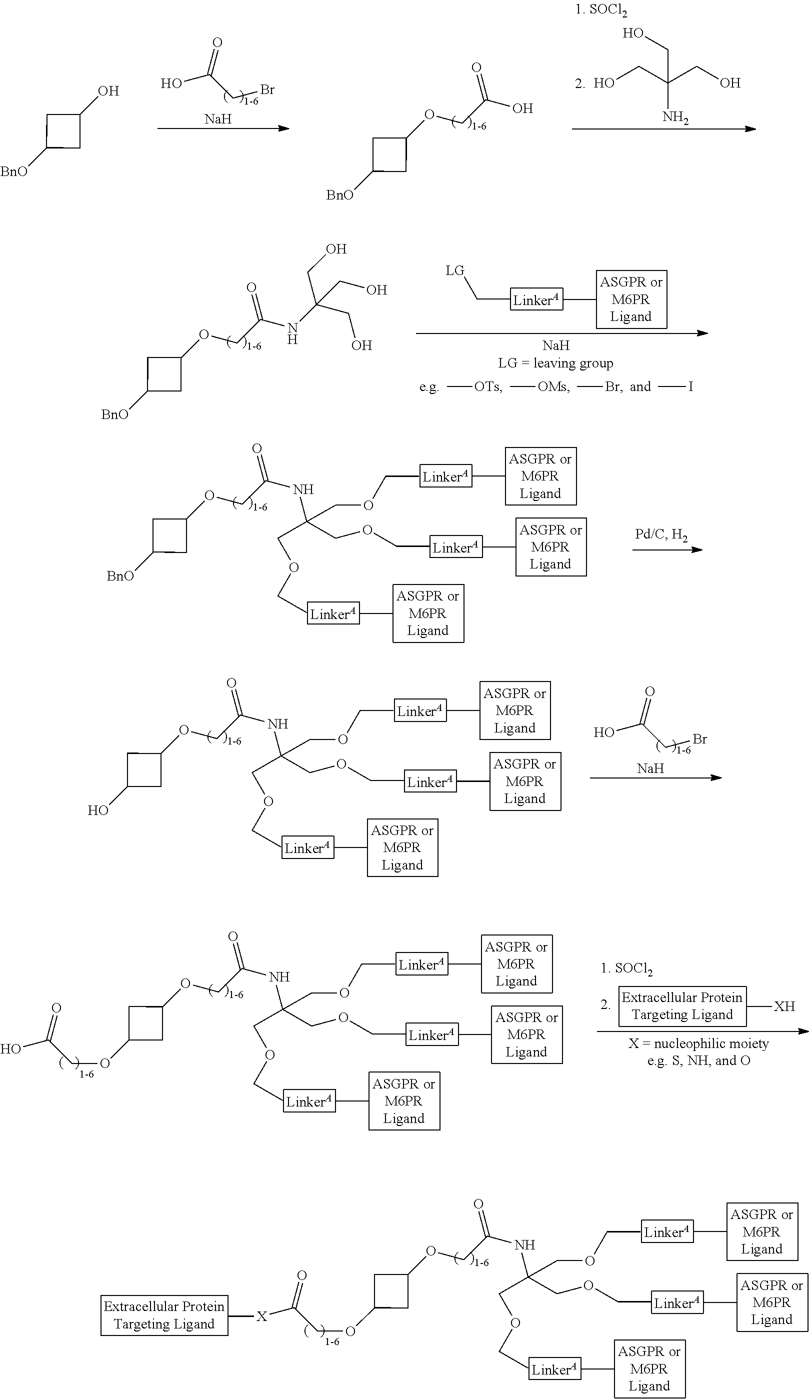

### For Polyethylene Glycol:

### Linker^{C}:

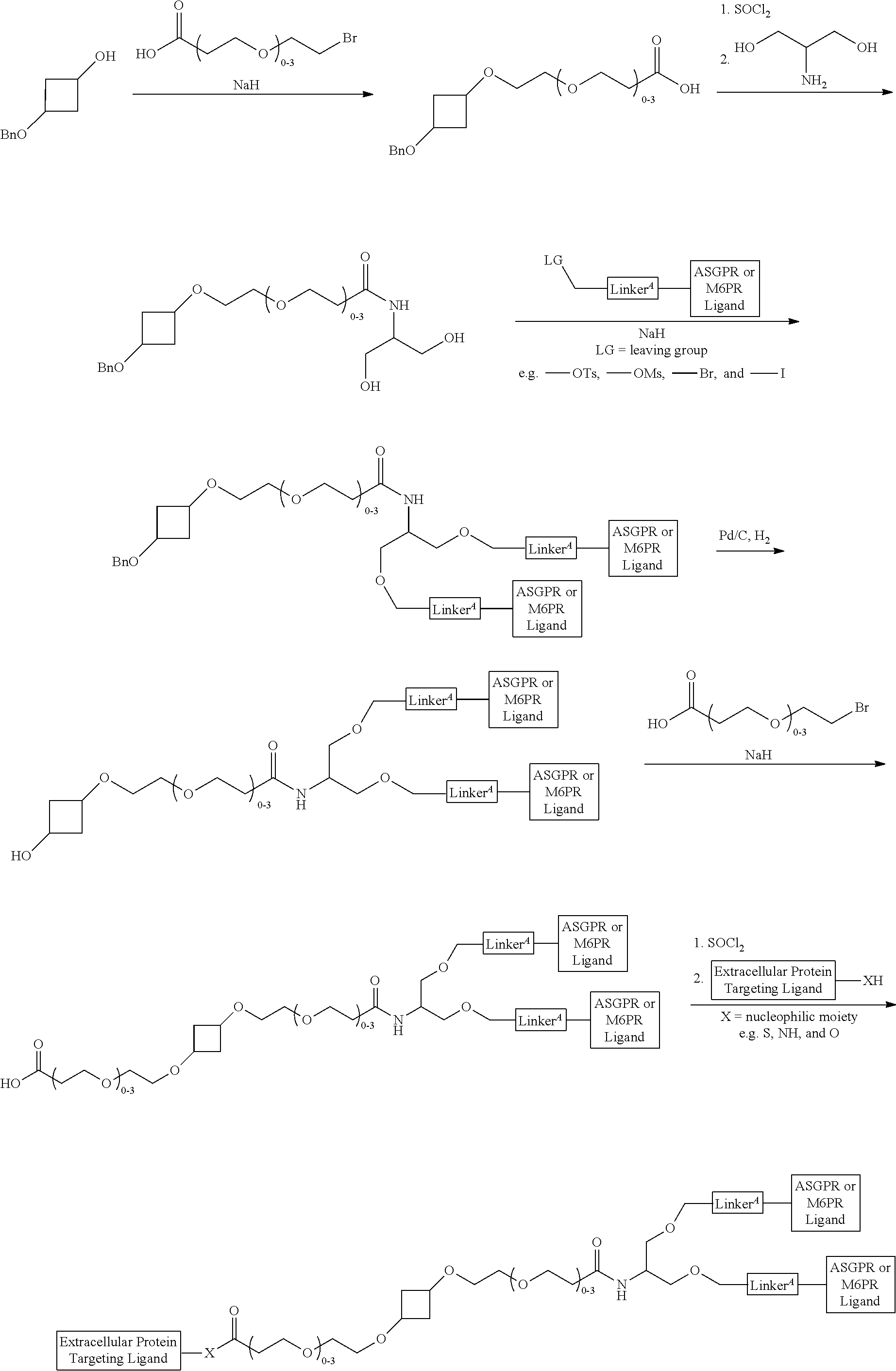

### Linker^{D}:

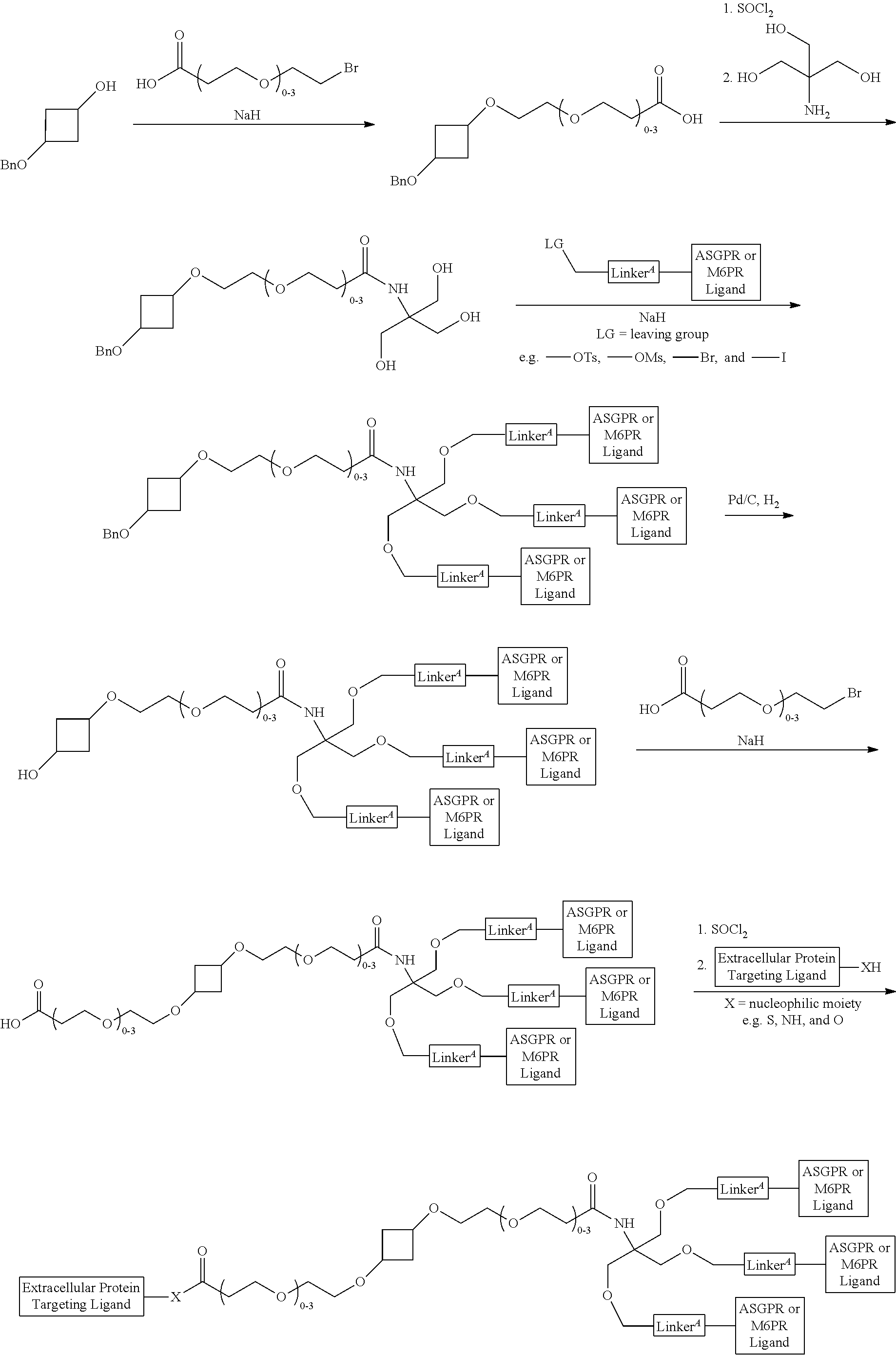

### Alternatively, for Linear Alkyl:

### Linker^{C}:

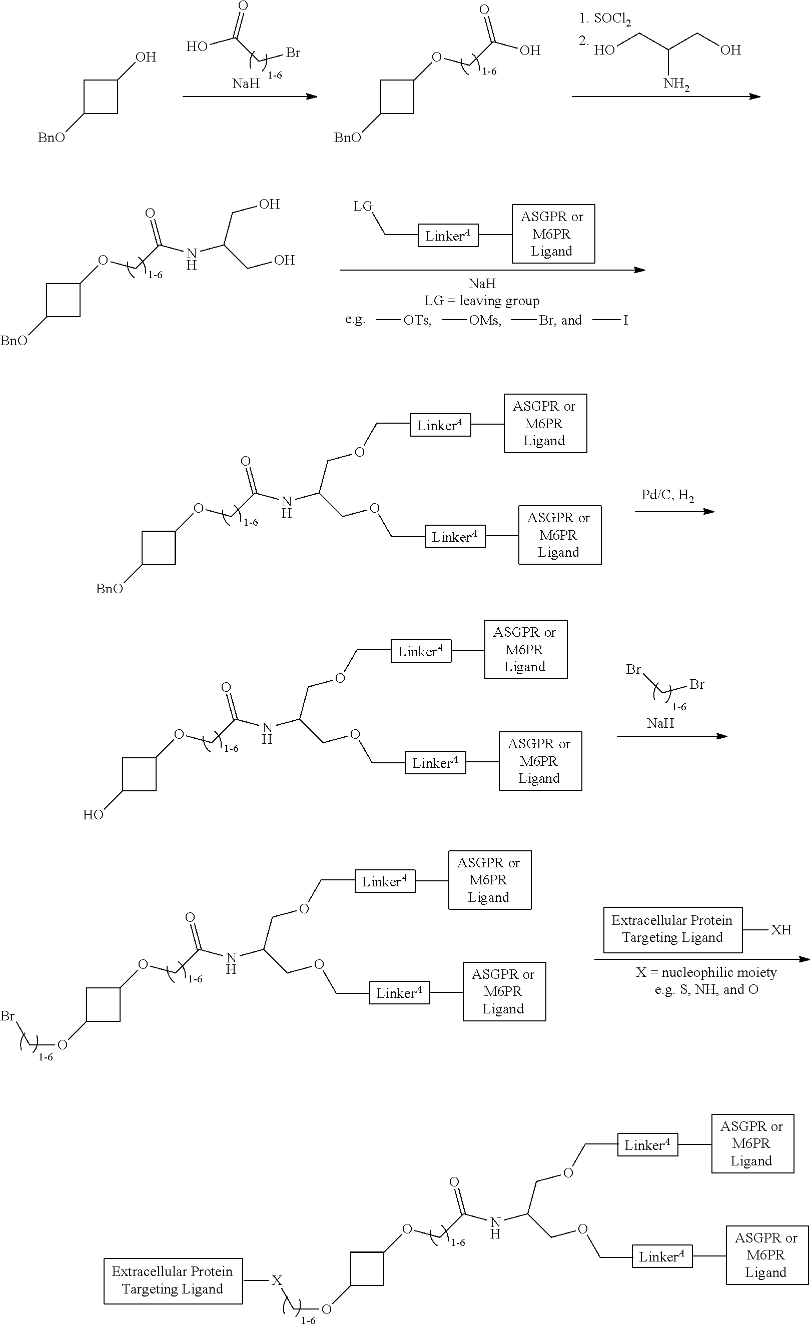

### Linker^{D}:

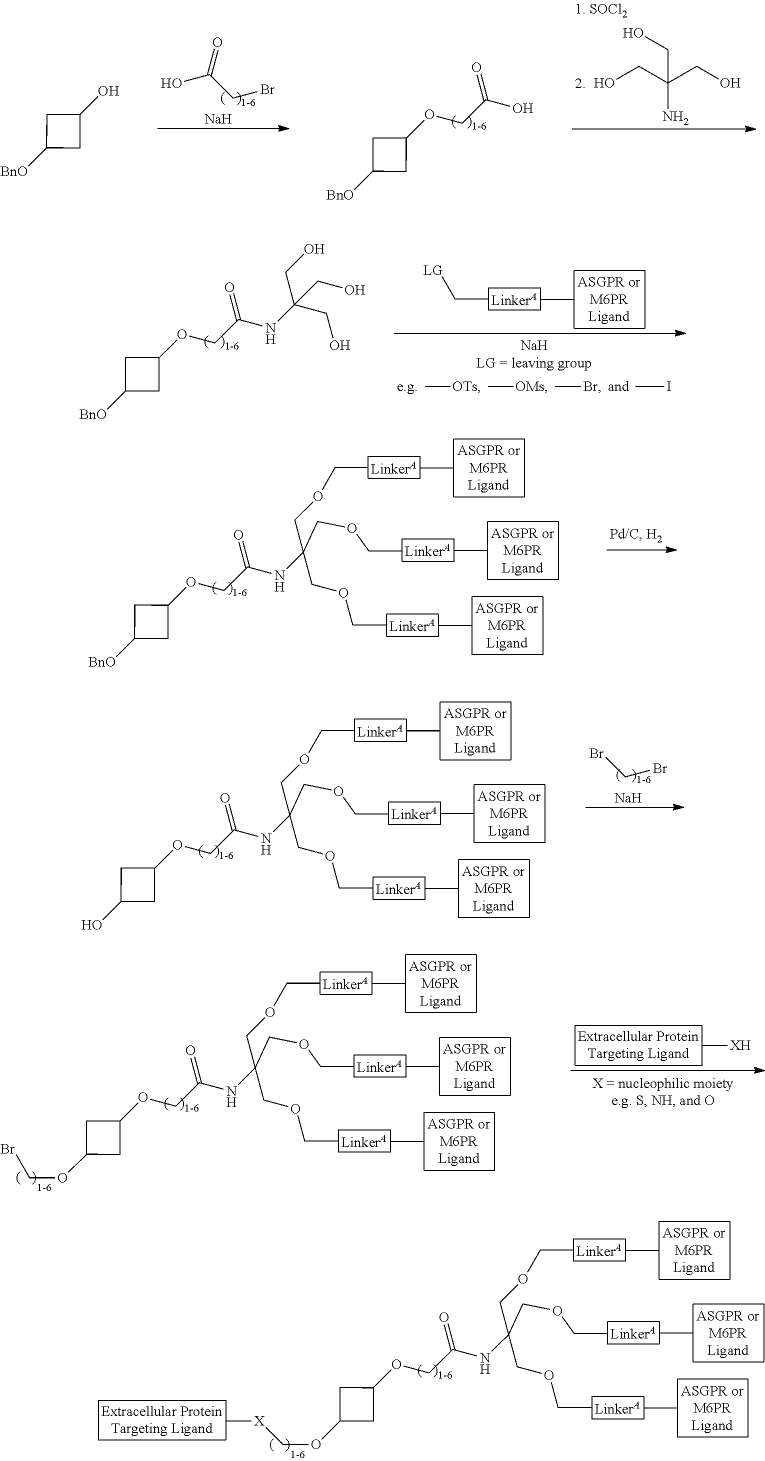

### Alternatively, for Polyethylene Glycol:

### Linker^{C}:

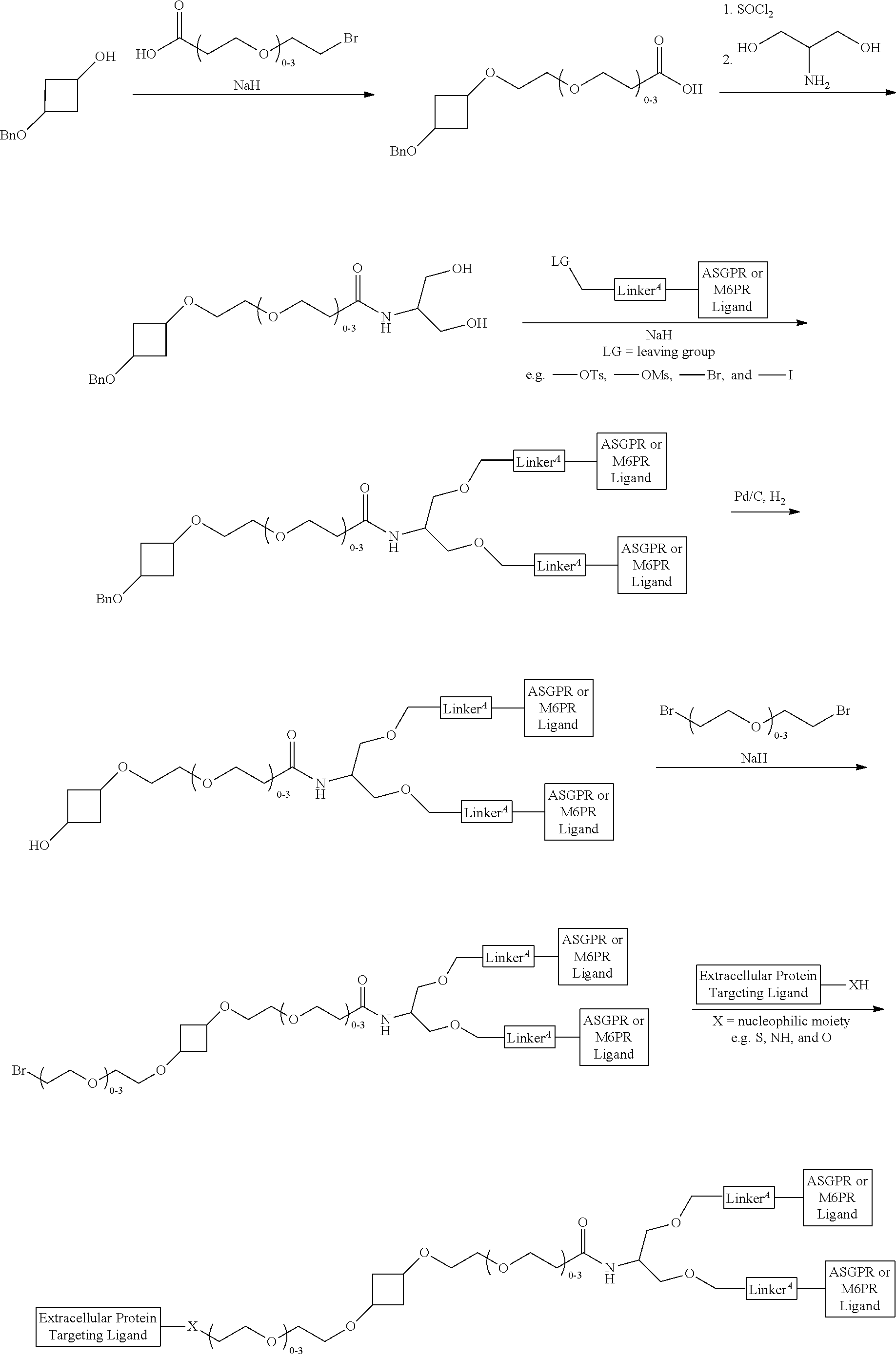

### Linker^{D}:

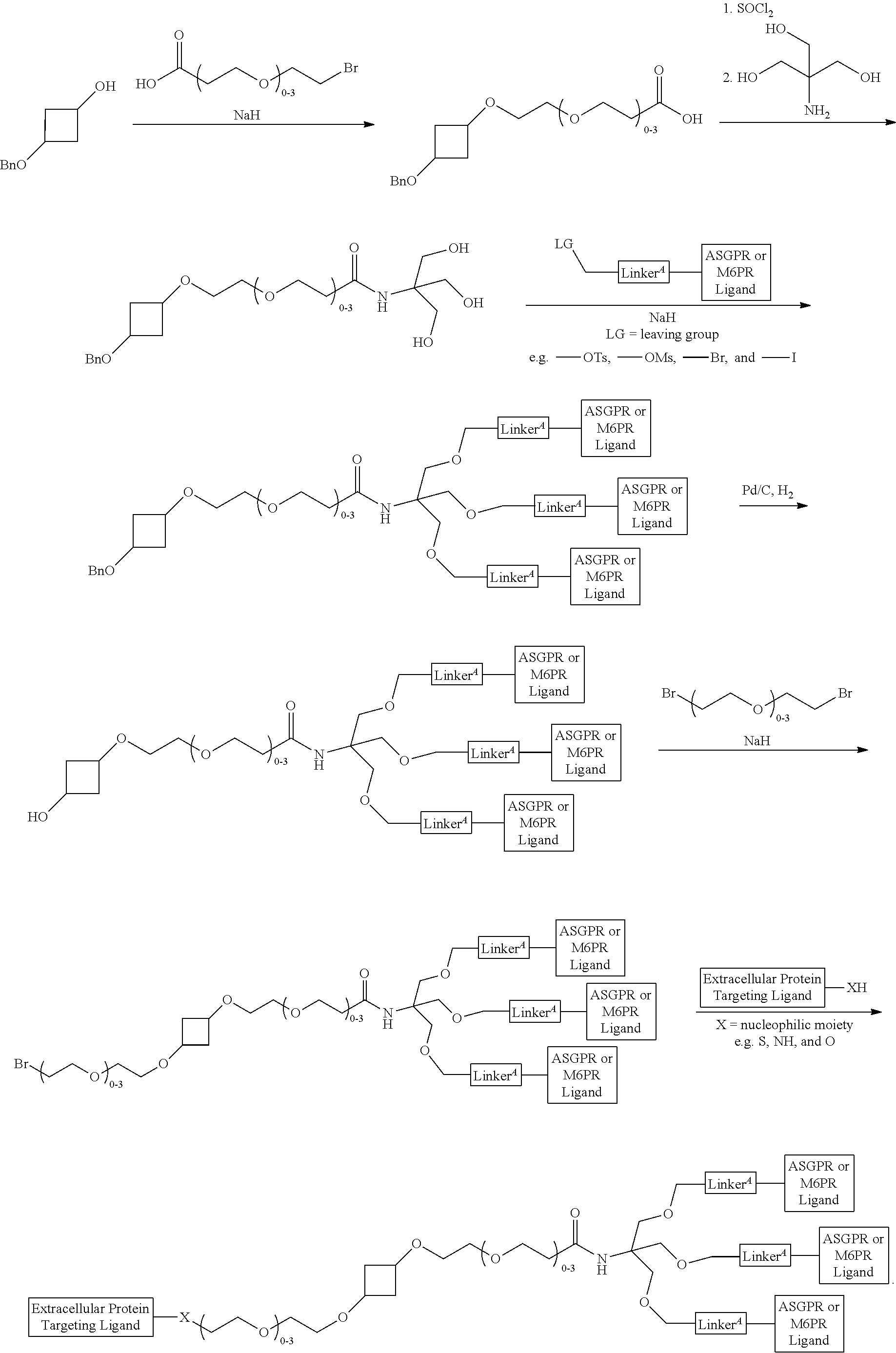

All publications and patent applications cited in this specification are herein incorporated by reference as if each individual publication or patent application were specifically and individually indicated to be incorporated by reference.

Although the foregoing invention has been described in some detail by way of illustration and example for the purpose of clarity of understanding, it will be readily apparent to one of ordinary skill in the art in light of the teaching of this invention that certain changes or modifications may be made thereto without departing from the spirit or scope of the invention as defined in the appended claims. Additionally, those skilled in the art will recognize, or be able to ascertain using no more than routine experimentation, many equivalents to the specific embodiments and methods described herein. Such equivalents are intended to be encompassed by the scope of the present application.

We claim:

1. A compound of Formula: 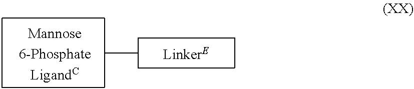
or a salt thereof,
wherein:
Mannose 6-Phosphate Ligand^{C} is selected from the group consisting of: 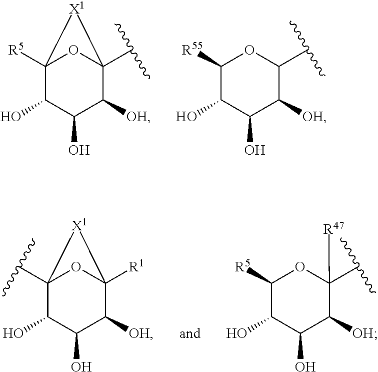
wherein
X¹ is 1 to 5 contiguous atoms independently selected from the group consisting of O, S, S(O), S(O)₂, N(R⁶), and C(R⁴)(R⁴), wherein if X¹ is 1 atom then X¹ is O, S, S(O), S(O)₂, N(R⁶), or C(R⁴)(R⁴), if X¹ is 2 atoms then no more than 1 atom of X¹ is O, S, or N(R⁶), if X¹ is 3, 4, or 5 atoms then no more than 2 atoms of X¹ are O, S, or N(R⁶);
R¹ is hydrogen, heteroalkyl, C₁-C₆alkyl-cyano, alkyl, alkenyl, alkynyl, haloalkyl, F, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-P(O)(R³)₂, C₁-C₆alkyl-OP(O)(R³)₂, C₀-C₆alkyl-NR⁸P(O)(R³)₂, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₁-C₆alkyl-O—C(O)R³, C₁-C₆alkyl-O—S(O)R³, C₀-C₆alkyl-O—C(S)R³, —N═S(O)(R³)₂, C₀-C₆alkylN₃, or C₁-C₆alkyl-O-S(O)₂R³;
R³ at each occurrence is independently selected from hydrogen, alkyl, heteroalkyl, haloalkyl, arylalkyl, heteroarylalkyl, alkenyl, alkynyl, aryl, heteroaryl, heterocycle, —OR⁸, and —NR⁸R⁹;
R⁴ is independently selected at each occurrence from hydrogen, halogen, heteroalkyl, alkyl, haloalkyl, arylalkyl, heteroarylalkyl, alkenyl, alkynyl, aryl, heteroaryl, heterocycle, —OR⁶, —NR⁶R⁷, C(O)R³, S(O)R³, C(S)R³, and S(O)₂R³;
R⁵ is selected from 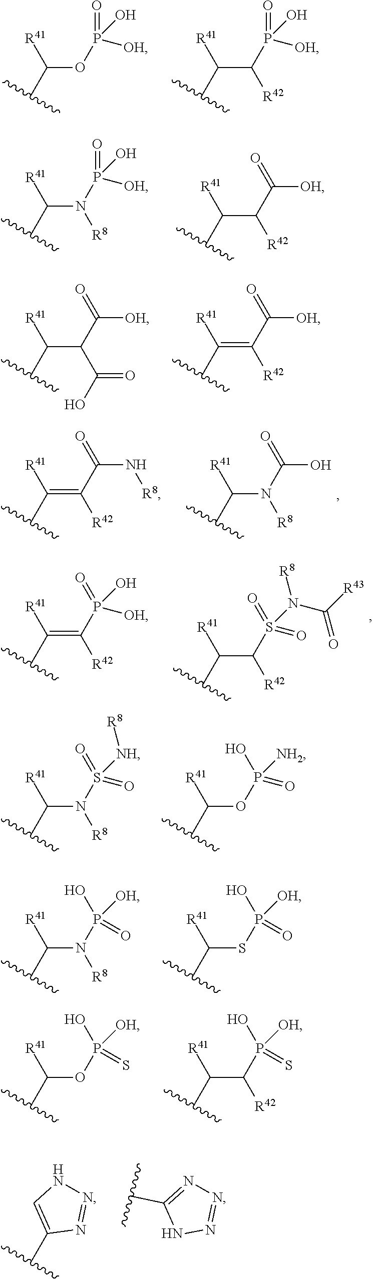
heteroaryl, heterocycle, and R⁵⁵;
R⁵⁵ is selected from 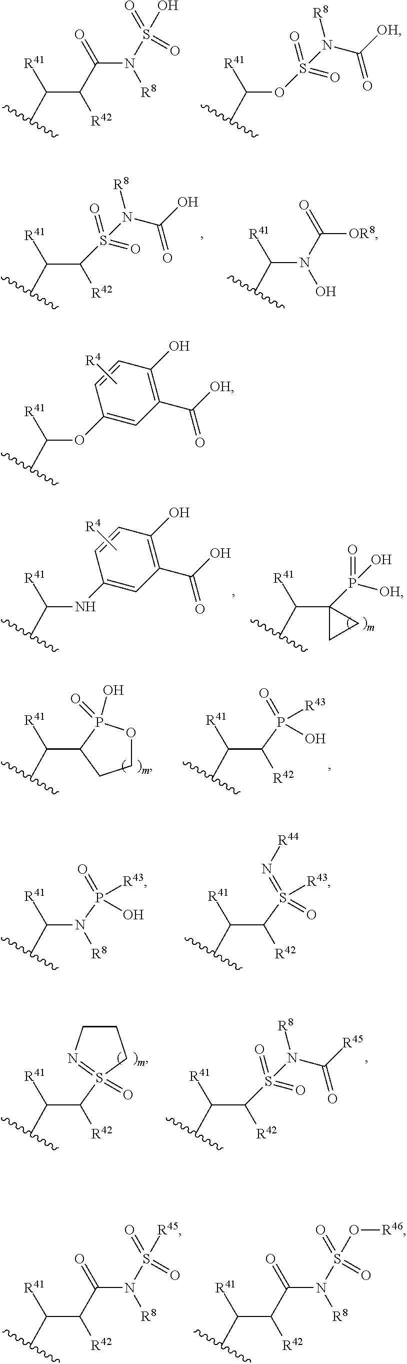 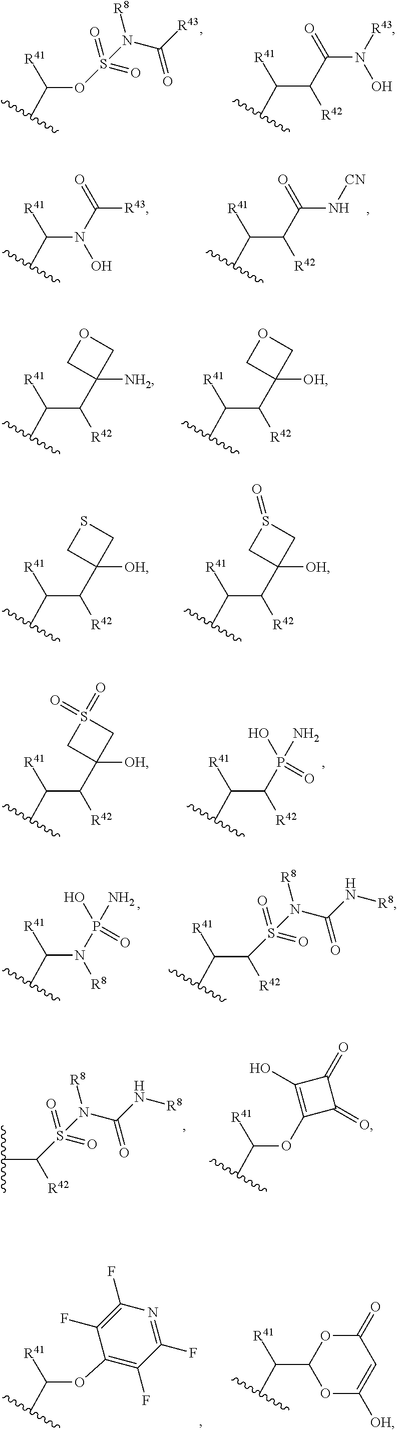 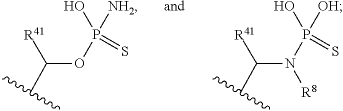
m is 1, 2, or 3;
R⁶ and R⁷ are independently selected at each occurrence from hydrogen, heteroalkyl, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, heteroalkyl, alkyl, arylalkyl, heteroarylalkyl, alkenyl, alkynyl, aryl, heteroaryl, and heterocycle;
R⁴¹ and R⁴² are independently selected from hydrogen, halogen, heteroalkyl, alkyl, arylalkyl, heteroarylalkyl, alkenyl, alkynyl, aryl, heteroaryl, and heterocycle;
R⁴³ is selected from alkyl, alkenyl, alkynyl, aryl, -alkyl-aryl, heteroaryl, -alkyl-heteroaryl, heterocycle, and -alkyl-heterocycle, each of which is optionally substituted with 1, 2, 3, or 4 substituents;
R⁴⁴ is selected from hydrogen, alkyl, cyano, alkenyl, alkynyl, aryl, heteroaryl, and heterocycle each of which except hydrogen and cyano is optionally substituted with 1, 2, or 3 substituents;
R⁴⁵ is selected from cycloalkyl, aryl, heteroaryl, and heterocycle each of which is optionally substituted with 1, 2, or 4 substituents;
R⁴⁶ is selected from hydrogen, alkyl, alkenyl, alkynyl, aryl, heteroaryl, and heterocycle each of which except hydrogen is optionally substituted with 1, 2, or 4 substituents;
Linker^{E} is selected from 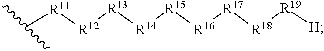
R¹¹, R¹², R¹³, R¹⁴, R¹⁵, R¹⁶, R¹⁷, R¹⁸, and R¹⁹ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —C(O)N(OH)—, —N(OH)C(O)—, —O—, —S—, —NR⁶—, —S(O)(═N—R⁴⁴)—, —S(O)(═NR), —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1 or 2 substituents independently selected from R²¹;
n is independently selected at each instance from 0, 1, 2, 3, 4, or 5;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, aryl, heteroaryl, and heterocycle, wherein the 1 or 2 R²¹ substituents are selected such that a stable compound results; and
each optional substituent is independently selected from alkyl, alkenyl, alkynyl, haloalkyl, —OR⁶, F, Cl, —NR⁶R⁷, heteroalkyl, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, 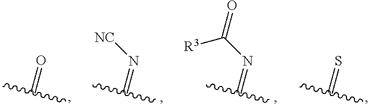
as allowed by valence wherein the optional substituent is selected such that a stable compound results.

2. The compound of claim 1, wherein R⁵ is selected from 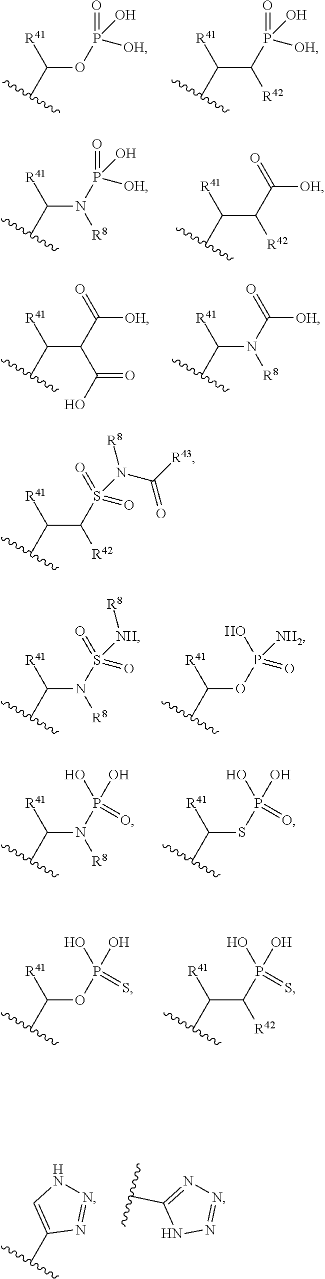
heteroaryl, heterocycle, and R⁵⁵.

3. The compound of claim 2, wherein R⁵ is selected from

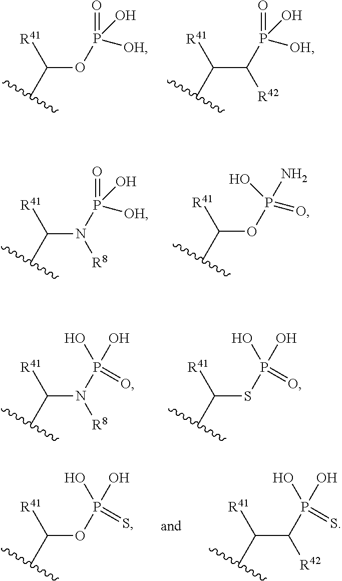

4. The compound of claim 2, wherein R⁵ is R⁵⁵.

5. The compound of claim 1, selected from

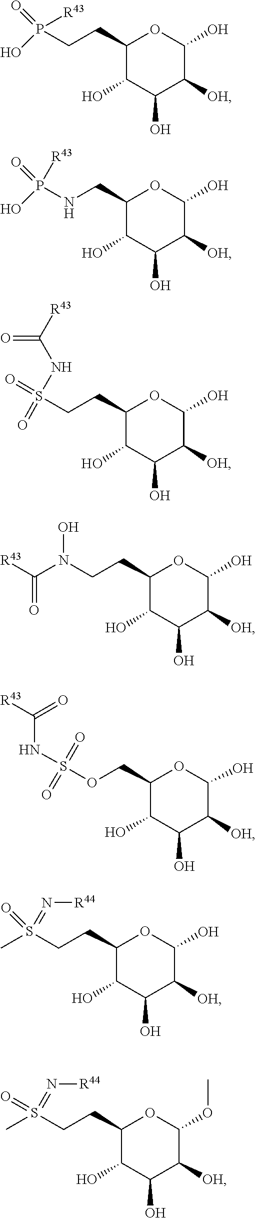

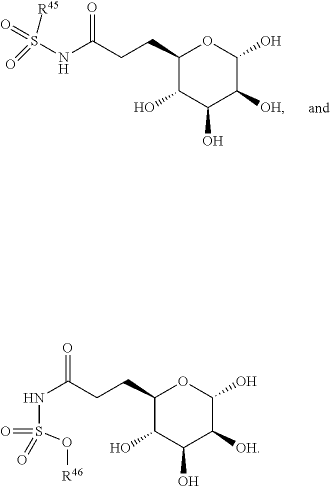

6. The compound of claim 5, wherein R⁴³ is selected from alkyl, alkenyl, alkynyl, aryl, -alkyl-aryl, heteroaryl, -alkyl-heteroaryl, heterocycle, and -alkyl-heterocycle each of which is optionally substituted with 1 or 2 substituents.

7. The compound of claim 5, wherein R⁴⁴ is selected from hydrogen, alkyl, cyano, alkenyl, alkynyl, aryl, heteroaryl, and heterocycle each of which except hydrogen is optionally substituted with 1 or 2 substituents.

8. The compound of claim 5, wherein R⁴⁶ is selected from hydrogen, alkyl, alkenyl, aryl, heteroaryl, and heterocycle each of which except hydrogen is optionally substituted with 1 or 2 substituents.

9. The compound of claim 1, wherein R¹¹, R¹², R¹³, R¹⁴, R¹⁵, R¹⁶, R¹⁷, R¹⁸, and R¹⁹ are independently at each occurrence selected from the group consisting of a bond, alkyl, —C(O)—, —C(O)O—, —OC(O)—, —SO₂—, —S(O)—, —C(O)NR⁶—, —NR⁶C(O)—, —O—, —S—, —NR⁶—, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, each of which is optionally substituted with 1 or 2 substituents independently selected from R²¹.

10. The compound of claim 9, wherein R¹¹, R¹², R¹³, R¹⁴, R¹⁵, R¹⁶, R¹⁷, R¹⁸, and R¹⁹ are independently at each occurrence selected from the group consisting of a bond, alkyl, —C(O)—, —C(O)O—, —OC(O)—, —SO₂—, —S(O)—, —C(O)NR⁶—, —NR⁶C(O)—, —O—, —S—, —NR⁶—, alkenyl, alkynyl, haloalkyl, alkoxy, aryl, heterocycle, and heteroaryl; each of which is optionally substituted with 1 or 2 substituents independently selected from R²¹.

11. The compound of claim 9, wherein R¹¹, R¹², R¹³, R¹⁴, R¹⁵, R¹⁶, R¹⁷, R¹⁸, and R¹⁹ are independently at each occurrence selected from the group consisting of a bond, alkyl, —C(O)—, —C(O)O—, —OC(O)—, —SO₂—, —S(O)—, —C(O)NR⁶—, —NR⁶C(O)—, —O—, —S—, and —NR⁶—.

12. A compound of Formula: 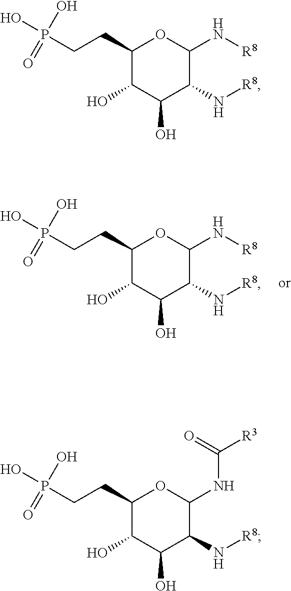
or a salt thereof,
wherein:
R³ at each occurrence is independently selected from hydrogen, alkyl, heteroalkyl, haloalkyl, arylalkyl, heteroarylalkyl, alkenyl, alkynyl, aryl, heteroaryl, heterocycle, —OR⁸, and —NR⁸R⁹; and
R⁸ are R⁹ are independently selected at each occurrence from hydrogen, heteroalkyl, alkyl, arylalkyl, heteroarylalkyl, alkenyl, alkynyl, aryl, heteroaryl, and heterocycle.

13. The compound of claim 12, wherein R⁸ and R⁹ are independently selected at each occurrence from hydrogen, alkyl, aryl, heteroaryl, and heterocycle.

14. The compound of claim 12, wherein R⁸ and R⁹ are independently selected at each occurrence from hydrogen, and alkyl.

15. The compound of claim 12, wherein R⁸ and R⁹ are both hydrogen.

16. The compound of claim 1, wherein the compound is of Formula: 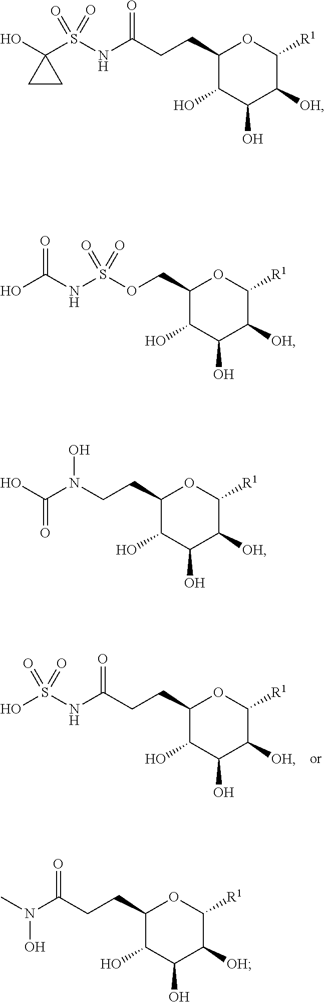
or a salt thereof.

17. The compound of claim 16, wherein R¹ is selected from hydrogen, methoxy, C₀-C₆alkyl-OR⁶, C₀-C₆alkyl-NR⁶R⁷, alkyl, heteroalkyl, alkenyl, alkynyl, aryl and heteroaryl, wherein alkyl, heteroalkyl, alkenyl, alkynyl, aryl and heteroaryl is optionally substituted with 1 or 2 substituents.

18. The compound of claim 16, wherein R¹ is selected from chloro, bromo, C₀-C₆alkyl-O—C(O)R³, C₁-C₆alkylN₃, and C₀-C₆alkyl-O—S(O)₂R³.

19. The compound of claim 1, wherein the compound of selected from 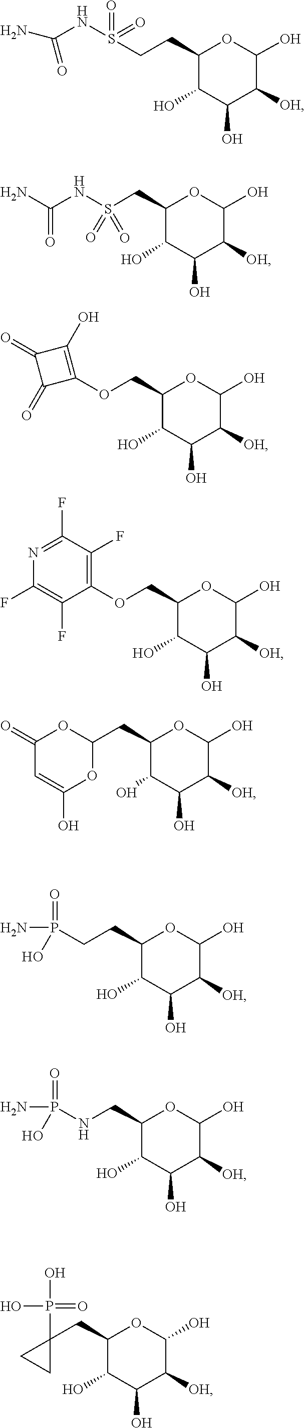 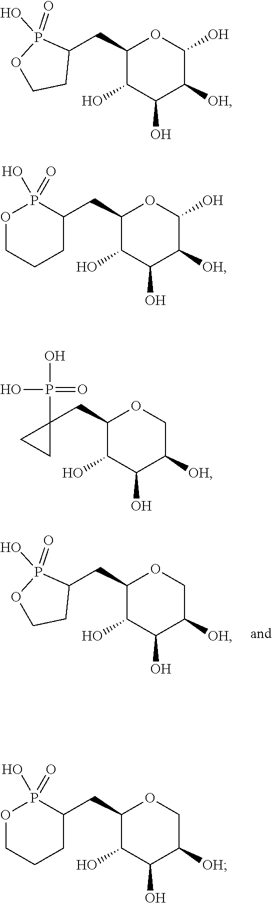 [SMILES: O=P1(O)OCCC1C[C@H]1O[C@H](O)[C@@H](O)[C@@H](O)[C@@H]1O ; O=P1(O)OCCCC1C[C@H]1O[C@H](O)[C@@H](O)[C@@H](O)[C@@H]1O ; O=P(O)(O)C1(C[C@H]2OC[C@@H](O)[C@@H](O)[C@@H]2O)CC1 ; O=P1(O)OCCC1C[C@H]1OC[C@@H](O)[C@@H](O)[C@@H]1O ; O=P1(O)OCCCC1C[C@H]1OC[C@@H](O)[C@@H](O)[C@@H]1O]
or a salt thereof.

20. The compound of claim 1, wherein the compound is selected from 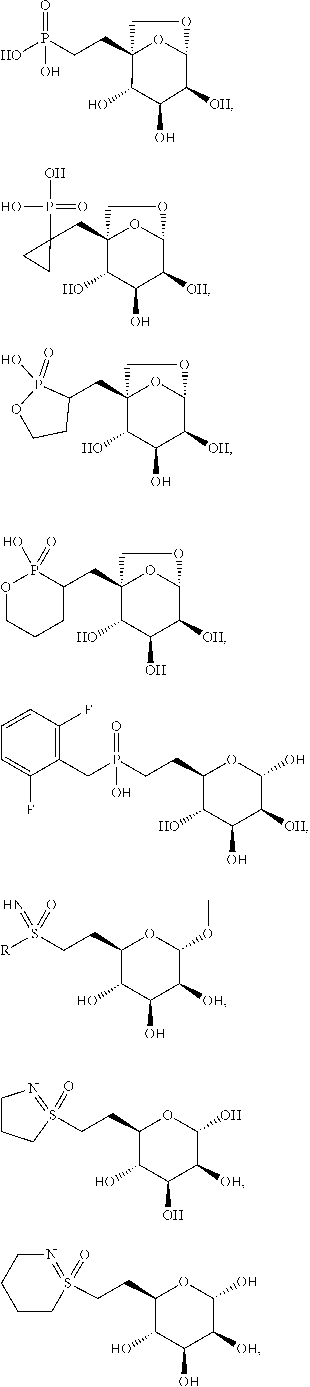 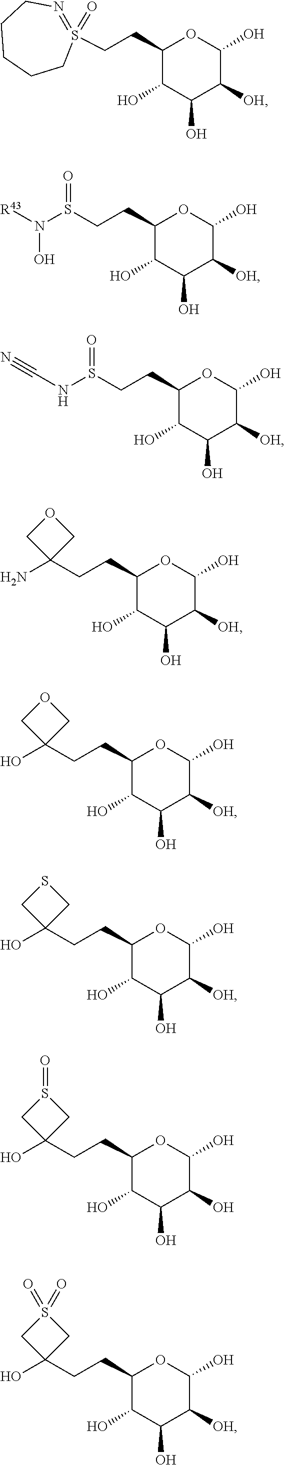 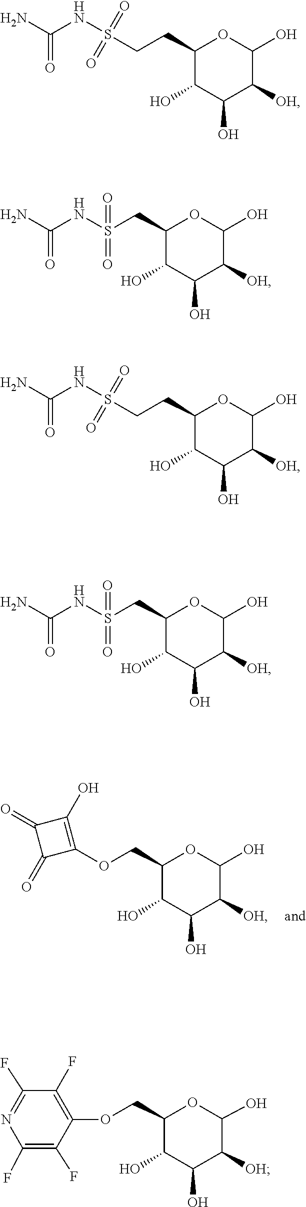 [SMILES: NC(=O)NS(=O)(=O)CC[C@H]1OC(O)[C@@H](O)[C@@H](O)[C@@H]1O ; NC(=O)NS(=O)(=O)C[C@H]1OC(O)[C@@H](O)[C@@H](O)[C@@H]1O ; NC(=O)NS(=O)(=O)CC[C@H]1OC(O)[C@@H](O)[C@@H](O)[C@@H]1O ; NC(=O)NS(=O)(=O)C[C@H]1OC(O)[C@@H](O)[C@@H](O)[C@@H]1O ; O=c1c(O)c(OC[C@H]2OC(O)[C@@H](O)[C@@H](O)[C@@H]2O)c1=O ; OC1O[C@H](COc2c(F)c(F)nc(F)c2F)[C@@H](O)[C@H](O)[C@@H]1O]
or a salt thereof.


SEQUENCE LISTING (390 sequences)

SEQ ID NO: 1 - protein, 6 aa, synthetic construct
MLKKIE

SEQ ID NO: 2 - protein, 19 aa, synthetic construct
VEPNCDIHVMWEWECFERL

SEQ ID NO: 3 - protein, 13 aa, synthetic construct
TVFTSWEEYLDWV

SEQ ID NO: 4 - protein, 13 aa, synthetic construct
  MOD_RES 7: note = N-Methylation
  MOD_RES 9: note = N-Methylation
  CROSSLNK 1..13: note = N-Terminal Acetyl to C-terminal Cysteine
  SITE 1: note = N-Terminal Acetyl
  SITE 6: note = L-4-phenyl-phenylalanine
  SITE 8: note = L-4-phenyl-phenylalanine
FVPTTXAXAEAPC

SEQ ID NO: 5 - protein, 13 aa, synthetic construct
  MOD_RES 7: note = N-Methylation
  MOD_RES 9: note = N-Methylation
  SITE 8: note = L-4-phenyl-phenylalanine
  CROSSLNK 1..13: note = N-Terminal Acetyl to C-Terminal Cysteine
  SITE 1: note = N-Terminal Acetylation
FVDTTSFXENSPC

SEQ ID NO: 6 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-terminal Cysteine
FVSTTXAXADRPC

SEQ ID NO: 7 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-terminal Cysteine
FVDTTSFXANSPC

SEQ ID NO: 8 - protein, 13 aa, synthetic construct
  MOD_RES 7: note = N-Methylation
  SITE 8: note = L-4-phenyl-phenylalanine
  MOD_RES 9: note = N-Methylation
  SITE 1: note = N-Terminal Acetyl
  CROSSLNK 1..13: note = N-Terminal Acetyl to C-terminal Cysteine
FVDSTTAXANHPC

SEQ ID NO: 9 - protein, 13 aa, synthetic construct
  SITE 1: note = N-Terminal Acetyl
  MOD_RES 7: note = N-Methylation
  MOD_RES 9: note = N-Methylation
  SITE 8: note = L-4-phenyl-phenylalanine
  CROSSLNK 1..13: note = N-Terminal Acetyl to C-Terminal Cysteine
FVDTTSFXAESPC

SEQ ID NO: 10 - protein, 13 aa, synthetic construct
  SITE 1: note = N-Terminal Acetyl
  CROSSLNK 1..13: note = N-Terminal Acetyl to C-Terminal Cysteine
  MOD_RES 7: note = N-Methylation
  MOD_RES 8: note = 4CF
  MOD_RES 9: note = N-Methylation
FVDTTSFFAESPC

SEQ ID NO: 11 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVDTTSAXAKSPC

SEQ ID NO: 12 - protein, 13 aa, synthetic construct
  SITE 1: note = N-Terminal Acetyl
  MOD_RES 7: note = N-Methylation
  SITE 6: note = L-4-phenyl-phenylalanine
  SITE 8: note = L-4-phenyl-phenylalanine
  MOD_RES 9: note = L-4-phenyl-phenylalanine
  CROSSLNK 1..13: note = N-Terminal Acetyl to C-Terminal Cysteine
FVSTTXAXADSPC

SEQ ID NO: 13 - protein, 14 aa, synthetic construct
  MOD_RES 7: note = N-Methylation
  SITE 1: note = N-Terminal Acetyl
  SITE 8: note = L-4-phenyl-phenylalanine
  MOD_RES 9: note = N-Methylation
  CROSSLNK 1..13: note = N-Terminal Acetyl to C-Terminal Cysteine
FVSTTSFXEADRPC

SEQ ID NO: 14 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVDTTXAXAESPC

SEQ ID NO: 15 - protein, 13 aa, synthetic construct
  SITE 1: note = N-Terminal Acetyl
  MOD_RES 6: note = 4CF3
  MOD_RES 7: note = N-Methylation
  MOD_RES 8: note = 4CF3
  MOD_RES 9: note = N-Methylation
  CROSSLNK 1..13: note = N-Terminal Acetyl to C-Terminal Cysteine
FVSTTFAFAERPC

SEQ ID NO: 16 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTSFXAESPC

SEQ ID NO: 17 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTXFXAESPC

SEQ ID NO: 18 - protein, 13 aa, synthetic construct
  SITE 1: note = N-Terminal Acetylation
  SITE 6: note = L-4-phenyl-phenylalanine
  MOD_RES 7: note = N-Methylation
  MOD_RES 8: note = 3,4diCI
  MOD_RES 9: note = N-Methylation
  CROSSLNK 1..13: note = N-Terminal Acetyl to C-Terminal Cysteine
FVSTTXAFADRPC

SEQ ID NO: 19 - protein, 13 aa, synthetic construct
  SITE 1: note = N-Terminal Acetyl
  SITE 6: note = L-4-phenyl-phenylalanine
  MOD_RES 7: note = N-Methylation
  MOD_RES 8: note = 3FI
  MOD_RES 9: note = N-Methylation
  CROSSLNK 1..13: note = N-Terminal Acetyl to C-Terminal Cysteine
FVSTTXAFADRPC

SEQ ID NO: 20 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVDTTAFXAEAPC

SEQ ID NO: 21 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = L-4-phenyl-phenylalanine
  CROSSLNK 1..13: note = N-Terminal Acetyl to C-Terminal Cysteine
FVDTTFAXAESPC

SEQ ID NO: 22 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVDTTAFXAQAPC

SEQ ID NO: 23 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVPTTXAXADRPC

SEQ ID NO: 24 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTXAXAPSPC

SEQ ID NO: 25 - protein, 13 aa, synthetic construct
  SITE 1: note = N-Terminal Acetyl
  SITE 8: note = L-4-phenyl-phenylalanine
  MOD_RES 7: note = N-Methylation
  MOD_RES 9: note = N-Methylation
  CROSSLNK 1..13: note = N-Terminal Acetyl to C-Terminal Cysteine
FVPTTAFXAEAPC

SEQ ID NO: 26 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVATTFAXAKAPC

SEQ ID NO: 27 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVNTTFAXAKAPC

SEQ ID NO: 28 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTFAXAEAPC

SEQ ID NO: 29 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTXAXAESPC

SEQ ID NO: 30 - protein, 13 aa, synthetic construct
  SITE 1: note = N-Terminal Acetyl
  SITE 6: note = L-4-phenyl-phenylalanine
  MOD_RES 7: note = N-Methylation
  MOD_RES 8: note = 3,4diCI
  MOD_RES 9: note = N-Methylation
  CROSSLNK 1..13: note = N-Terminal Acetyl to C-Terminal Cysteine
FVDTTXAFAESPC

SEQ ID NO: 31 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FENTTFAXAASPC

SEQ ID NO: 32 - protein, 13 aa, synthetic construct
  SITE 1: note = N-Terminal Acetyl
  MOD_RES 7: note = N-Methylation
  MOD_RES 8: note = 4CI
  MOD_RES 9: note = N-Methylation
  CROSSLNK 1..13: note = N-Terminal Acetyl to C-Terminal Cysteine
FVPTTFAFAPAPC

SEQ ID NO: 33 - protein, 13 aa, synthetic construct
  SITE 1: note = N-Terminal Acetyl
  MOD_RES 7: note = N-Methylation
  MOD_RES 8: note = 4CI
  MOD_RES 9: note = N-Methylation
  CROSSLNK 1..13: note = N-Terminal Acetyl to C-Terminal Cysteine
FVPTTFAFADAPC

SEQ ID NO: 34 - protein, 13 aa, synthetic construct
  SITE 1: note = N-Terminal Acetyl
  SITE 3: note = Hse
  MOD_RES 7: note = N-Methylation
  MOD_RES 8: note = 4CI
  MOD_RES 9: note = N-Methylation
  CROSSLNK 1..13: note = N-Terminal Acetyl to C-Terminal Cysteine
FVXTTFAFADAPC

SEQ ID NO: 35 - protein, 13 aa, synthetic construct
  SITE 1: note = N-Terminal Acetyl
  MOD_RES 7: note = N-Methylation
  MOD_RES 8: note = 4CI
  MOD_RES 9: note = N-Methylation
  CROSSLNK 1..13: note = N-Terminal Acetyl to C-Terminal Cysteine
FVATTFAFANAPC

SEQ ID NO: 36 - protein, 13 aa, synthetic construct
  SITE 1: note = N-Terminal Acetyl
  MOD_RES 7: note = N-Methylation
  MOD_RES 8: note = 4CI
  MOD_RES 9: note = N-Methylation
  CROSSLNK 1..13: note = N-Terminal Acetyl to C-Terminal Cysteine
FVPTTVAFAEAPC

SEQ ID NO: 37 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  MOD_RES 10: note = N-Methylation
  CROSSLNK 1..13: note = N-Terminal Acetyl to C-Terminal Cysteine
FVNTTFAXAEAPC

SEQ ID NO: 38 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVPTTFAXAEAPC

SEQ ID NO: 39 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVPTTXAXAAAPC

SEQ ID NO: 40 - protein, 16 aa, synthetic construct
HMVCLAYRGRPVCFAL

SEQ ID NO: 41 - protein, 16 aa, synthetic construct
HMVCLSYRGRPVCFSL

SEQ ID NO: 42 - protein, 16 aa, synthetic construct
HQVCLSYRGRPVCFST

SEQ ID NO: 43 - protein, 16 aa, synthetic construct
QMRCLSYKGRRVCLWL

SEQ ID NO: 44 - protein, 16 aa, synthetic construct
KRLCLQYKGSKVCFRL

SEQ ID NO: 45 - protein, 16 aa, synthetic construct
RMRCLTYRGRRVCLEL

SEQ ID NO: 46 - protein, 16 aa, synthetic construct
SMRCLQYRGSRVCLTL

SEQ ID NO: 47 - protein, 16 aa, synthetic construct
HLRCLRYKGTRVCFSL

SEQ ID NO: 48 - protein, 16 aa, synthetic construct
HVRCLSYKGREVCVQL

SEQ ID NO: 49 - protein, 16 aa, synthetic construct
PRMCLFIYKGRRVCIP

SEQ ID NO: 50 - protein, 16 aa, synthetic construct
HMRCLHYKGRRVCFLL

SEQ ID NO: 51 - protein, 16 aa, synthetic construct
HKRCLHYRGRMVCFLI

SEQ ID NO: 52 - protein, 16 aa, synthetic construct
QKRCLKYKGSRVCFFL

SEQ ID NO: 53 - protein, 16 aa, synthetic construct
HVRCLRYRGKNVCFLL

SEQ ID NO: 54 - protein, 16 aa, synthetic construct
SDVCLRYRGRPVCFQV

SEQ ID NO: 55 - protein, 16 aa, synthetic construct
RDVCLRYRGRPVCFQV

SEQ ID NO: 56 - protein, 16 aa, synthetic construct
HDVCLRYRGRPVCFQV

SEQ ID NO: 57 - protein, 16 aa, synthetic construct
SMVCLRYRGRPVCFQV

SEQ ID NO: 58 - protein, 16 aa, synthetic construct
SAVCLRYRGRPVCFQV

SEQ ID NO: 59 - protein, 16 aa, synthetic construct
SDVCLNYRGRPVCFQV

SEQ ID NO: 60 - protein, 16 aa, synthetic construct
SDVCLHYRGRPVCFQV

SEQ ID NO: 61 - protein, 16 aa, synthetic construct
SDVCLAYRGRPVCFQV

SEQ ID NO: 62 - protein, 16 aa, synthetic construct
SDVCLRYRGRPVCFAV

SEQ ID NO: 63 - protein, 16 aa, synthetic construct
SDVCLRYRGRPVCFQL

SEQ ID NO: 64 - protein, 16 aa, synthetic construct
SDVCLRYRGRPVCFQA

SEQ ID NO: 65 - protein, 14 aa, synthetic construct
HMVCLSYRGRPVCF

SEQ ID NO: 66 - protein, 15 aa, synthetic construct
HMVCLSYRGRPVCFS

SEQ ID NO: 67 - protein, 16 aa, synthetic construct
HQVCLSYRGQPVCFSL

SEQ ID NO: 68 - protein, 16 aa, synthetic construct
HQVCLSYRGRPTCFSL

SEQ ID NO: 69 - protein, 16 aa, synthetic construct
HQVCLSYRGRPVCYSL

SEQ ID NO: 70 - protein, 16 aa, synthetic construct
HQVCLSYRGQPVCFST

SEQ ID NO: 71 - protein, 16 aa, synthetic construct
HQVCLSYRGRPTCFST

SEQ ID NO: 72 - protein, 16 aa, synthetic construct
HQVCLSYRGQPTCFST

SEQ ID NO: 73 - protein, 16 aa, synthetic construct
  CROSSLNK 3..7: note = Tyr-Lys
  CROSSLNK 7..12: note = Lys-Lys
  CROSSLNK 11..16: note = Lys-Tyr
RTYRTYKRTYKKGRTY

SEQ ID NO: 74 - protein, 16 aa, synthetic construct
  SITE 1..16: note = D-amino acids
  CROSSLNK 3..7: note = Tyr-Lys
  CROSSLNK 11..16: note = Lys-Tyr
  CROSSLNK 7..12: note = Lys-Lys
RTYRTYKRTYKKGRTY

SEQ ID NO: 75 - protein, 16 aa, synthetic construct
  SITE 1..16: note = D-Amino Acids
  CROSSLNK 3..7: note = Tyr-Lys
  CROSSLNK 11..16: note = Lys-Tyr
  CROSSLNK 7..12: note = Lys-Lys
  MOD_RES 1: note = Phenylacetic acid
  MOD_RES 4: note = Phenylacetic acid
  MOD_RES 8: note = Phenylacetic acid
  MOD_RES 14: note = Phenylacetic acid
RTYRTYKRTYKKGRTY

SEQ ID NO: 76 - protein, 10 aa, synthetic construct
TWKTSRISIF

SEQ ID NO: 77 - protein, 10 aa, synthetic construct
FGRLVSSIRY

SEQ ID NO: 78 - protein, 13 aa, synthetic construct
  CROSSLNK 2..12: note = Cys-Cys
  SITE 13: note = C-Terminal Amide
DCAWHLGELVWCT

SEQ ID NO: 79 - protein, 12 aa, synthetic construct
DSAWHLGELWST

SEQ ID NO: 80 - protein, 13 aa, synthetic construct
DCHKRSFWADNCT

SEQ ID NO: 81 - protein, 13 aa, synthetic construct
DCRTQFRPNQTCT

SEQ ID NO: 82 - protein, 13 aa, synthetic construct
DCQLCDFWRTRCT

SEQ ID NO: 83 - protein, 13 aa, synthetic construct
DCFEDFNEQRTCT

SEQ ID NO: 84 - protein, 13 aa, synthetic construct
DCLAKFLKGKDCT

SEQ ID NO: 85 - protein, 13 aa, synthetic construct
DCWHRRTHKTFCT

SEQ ID NO: 86 - protein, 11 aa, synthetic construct
DCRTIQTRSCT

SEQ ID NO: 87 - protein, 13 aa, synthetic construct
DCIKLAQLHSVCT

SEQ ID NO: 88 - protein, 13 aa, synthetic construct
DCWRHRNATEWCT

SEQ ID NO: 89 - protein, 13 aa, synthetic construct
DCQNWIKDVHKCT

SEQ ID NO: 90 - protein, 13 aa, synthetic construct
DCAWHLGELVWCT

SEQ ID NO: 91 - protein, 13 aa, synthetic construct
DCAFHLGELVWCT

SEQ ID NO: 92 - protein, 13 aa, synthetic construct
DCAYHLGELVWCT

SEQ ID NO: 93 - protein, 11 aa, synthetic construct
  CROSSLNK 1..11: note = Pro-Pro
  SITE 11: note = D-Phenylalanine
PAWHLGELVWP

SEQ ID NO: 94 - protein, 15 aa, synthetic construct
  CROSSLNK 1..15: note = Pro-Pro
  CROSSLNK 3..13: note = Cys-Cys
  SITE 15: note = D-Amino Acid
PDCAWHLGELVWCTP

SEQ ID NO: 95 - protein, 15 aa, synthetic construct
  CROSSLNK 3..13: note = Cys-Cys
  CROSSLNK 1..15: note = Cys-Cys
CDCAWHLGELVWCTC

SEQ ID NO: 96 - protein, 12 aa, synthetic construct
EPIHRSTLTALL

SEQ ID NO: 97 - protein, 4 aa, synthetic construct
APAR

SEQ ID NO: 98 - protein, 6 aa, synthetic construct
  REPEAT 1..4: note = repeated twice
  CROSSLNK 4..5: note = His-Lys
  CROSSLNK 1: note = Cys-Cys
CFHHKG

SEQ ID NO: 99 - protein, 6 aa, synthetic construct
HWRGWV

SEQ ID NO: 100 - protein, 6 aa, synthetic construct
HYFKFD

SEQ ID NO: 101 - protein, 6 aa, synthetic construct
HFRRHL

SEQ ID NO: 102 - protein, 6 aa, synthetic construct
  NON_STD 3: note = Citrulline
HWXGWV

SEQ ID NO: 103 - protein, 6 aa, synthetic construct
  NON_STD 3: note = Citrulline
  MOD_RES 2: note = MeTrp
  MOD_RES 5: note = MeTrp
HWXGWV

SEQ ID NO: 104 - protein, 4 aa, synthetic construct
  REPEAT 1: note = repeated twice
  CROSSLNK 1..2: note = Asp-Ala
DAAG

SEQ ID NO: 105 - protein, 4 aa, synthetic construct
DAAG

SEQ ID NO: 106 - protein, 8 aa, synthetic construct
  NON_STD 1: note = (Nalpha-Ac)-S(A)
  NON_STD 8: note = Lact-E
  CROSSLNK 1..8: note = Nalpha-Ac-Lact-E
XRWHYFKX

SEQ ID NO: 107 - protein, 8 aa, synthetic construct
  CROSSLNK 1..8: note = Nalpha-Ac-Lact-E
  NON_STD 1: note = (Nalpha-Ac)-Dap(A)
  NON_STD 8: note = Lact-E
XRWHYFKX

SEQ ID NO: 108 - protein, 7 aa, synthetic construct
  CROSSLNK 1..7: note = Link M-Lys
  NON_STD 1: note = Link M
XWFRHYK

SEQ ID NO: 109 - protein, 8 aa, synthetic construct
NKFRGKYK

SEQ ID NO: 110 - protein, 8 aa, synthetic construct
NARKFYKG

SEQ ID NO: 111 - protein, 8 aa, synthetic construct
FYWHCLDE

SEQ ID NO: 112 - protein, 8 aa, synthetic construct
FYCHWALE

SEQ ID NO: 113 - protein, 8 aa, synthetic construct
FYCHTIDE

SEQ ID NO: 114 - protein, 16 aa, synthetic construct
FYWHCLDEFYCHTIDE

SEQ ID NO: 115 - protein, 4 aa, synthetic construct
RRGW

SEQ ID NO: 116 - protein, 7 aa, synthetic construct
KHRFNKD

SEQ ID NO: 117 - protein, 7 aa, synthetic construct
CPSTHWK

SEQ ID NO: 118 - protein, 7 aa, synthetic construct
NVQYFAV

SEQ ID NO: 119 - protein, 7 aa, synthetic construct
ASHTQKS

SEQ ID NO: 120 - protein, 7 aa, synthetic construct
QPQMSHM

SEQ ID NO: 121 - protein, 7 aa, synthetic construct
TNIESLK

SEQ ID NO: 122 - protein, 7 aa, synthetic construct
NCHKCWN

SEQ ID NO: 123 - protein, 7 aa, synthetic construct
SHLSKNF

SEQ ID NO: 124 - protein, 8 aa, synthetic construct
FDLLEHFY

SEQ ID NO: 125 - protein, 9 aa, synthetic construct
DLLHHFDYF

SEQ ID NO: 126 - protein, 13 aa, synthetic construct
GIFVIQSESLKKC

SEQ ID NO: 127 - protein, 9 aa, synthetic construct
SVLTLENCK

SEQ ID NO: 128 - protein, 8 aa, synthetic construct
SVLTLENC

SEQ ID NO: 129 - protein, 9 aa, synthetic construct
SVLTLDNCK

SEQ ID NO: 130 - protein, 8 aa, synthetic construct
SVLTEENC

SEQ ID NO: 131 - protein, 8 aa, synthetic construct
SVLTEENS

SEQ ID NO: 132 - protein, 8 aa, synthetic construct
SVLTDENC

SEQ ID NO: 133 - protein, 8 aa, synthetic construct
SVLTDENS

SEQ ID NO: 134 - protein, 9 aa, unidentified
PIQSESLKK

SEQ ID NO: 135 - protein, 9 aa, synthetic construct
VIDSESLKK

SEQ ID NO: 136 - protein, 9 aa, synthetic construct
PIDSESLKK

SEQ ID NO: 137 - protein, 9 aa, synthetic construct
VIQSESLKK

SEQ ID NO: 138 - protein, 17 aa, synthetic construct
  SITE 7: note = PEG
  SITE 9: note = PEG
PIESESXKXSVLTEENC

SEQ ID NO: 139 - protein, 17 aa, synthetic construct
  SITE 7: note = PEG
  SITE 9: note = PEG
VIQSESXKXSVLTLENC

SEQ ID NO: 140 - protein, 17 aa, synthetic construct
  SITE 7: note = PEG
  SITE 9: note = PEG
VIQSESXKXSVLTEENC

SEQ ID NO: 141 - protein, 17 aa, synthetic construct
  SITE 7: note = PEG
  SITE 9: note = PEG
PIDDESXKXSVLTLENC

SEQ ID NO: 142 - protein, 17 aa, synthetic construct
  SITE 7: note = PEG
  SITE 9: note = PEG
PIDDESXKXSVLTEENC

SEQ ID NO: 143 - protein, 19 aa, synthetic construct
VIQSESLKKCKSVLTLENC

SEQ ID NO: 144 - protein, 19 aa, synthetic construct
PIQSESLKKCKSVLTLENC

SEQ ID NO: 145 - protein, 19 aa, synthetic construct
VIESESLKKCKSVLTLENC

SEQ ID NO: 146 - protein, 19 aa, synthetic construct
VIDSESLKKCKSVLTLENC

SEQ ID NO: 147 - protein, 19 aa, synthetic construct
PIESESLKKCKSVLTLENC

SEQ ID NO: 148 - protein, 19 aa, synthetic construct
VIQSESLKKCIQAGKLENC

SEQ ID NO: 149 - protein, 19 aa, synthetic construct
PIQSESLKKCIQAGKLENC

SEQ ID NO: 150 - protein, 19 aa, synthetic construct
VIESESLKKCIQAGKLENC

SEQ ID NO: 151 - protein, 19 aa, synthetic construct
VIDSESLKKCIQAGKLENC

SEQ ID NO: 152 - protein, 19 aa, synthetic construct
PIESESLKKCIQAGKLENC

SEQ ID NO: 153 - protein, 19 aa, synthetic construct
PIQSESLKKCKSVLTLENK

SEQ ID NO: 154 - protein, 19 aa, synthetic construct
VIESESLKKCKSVLTLENK

SEQ ID NO: 155 - protein, 19 aa, synthetic construct
VIDSESLKKCKSVLTLENK

SEQ ID NO: 156 - protein, 19 aa, synthetic construct
PIESESLKKCKSVLTLENK

SEQ ID NO: 157 - protein, 19 aa, synthetic construct
VIQSESLKKCIQAGKLENK

SEQ ID NO: 158 - protein, 19 aa, synthetic construct
PIQSESLKKCIQAGKLENK

SEQ ID NO: 159 - protein, 19 aa, synthetic construct
VIESESLKKCIQAGKLENK

SEQ ID NO: 160 - protein, 19 aa, synthetic construct
VIDSESLKKCIQAGKLENK

SEQ ID NO: 161 - protein, 19 aa, synthetic construct
PIESESLKKCIQAGKLENK

SEQ ID NO: 162 - protein, 16 aa, synthetic construct
PIESESGSVLTLENCK

SEQ ID NO: 163 - protein, 17 aa, synthetic construct
PIESESGGSVLTLENCK

SEQ ID NO: 164 - protein, 18 aa, synthetic construct
PIESESGGGSVLTLENCK

SEQ ID NO: 165 - protein, 19 aa, synthetic construct
PIESESGGGGSVLTLENCK

SEQ ID NO: 166 - protein, 20 aa, synthetic construct
PIESESGGGGGSVLTLENCK

SEQ ID NO: 167 - protein, 16 aa, synthetic construct
VIQSESGSVLTLENCK

SEQ ID NO: 168 - protein, 17 aa, synthetic construct
VIQSESGGSVLTLENCK

SEQ ID NO: 169 - protein, 18 aa, synthetic construct
VIQSESGGGSVLTLENCK

SEQ ID NO: 170 - protein, 19 aa, synthetic construct
VIQSESGGGGSVLTLENCK

SEQ ID NO: 171 - protein, 20 aa, synthetic construct
VIQSESGGGGGSVLTLENCK

SEQ ID NO: 172 - protein, 28 aa, synthetic construct
KGCFVIQSESLKKSIQAGKSVLTLENCK

SEQ ID NO: 173 - protein, 21 aa, synthetic construct
LKKCIQAGKSVLTLENCKQAN

SEQ ID NO: 174 - protein, 22 aa, synthetic construct
WQDKGIFVIQSESLKKCIQAGK

SEQ ID NO: 175 - protein, 28 aa, synthetic construct
KGIFVIQSESLKKCIQAGKSVLTLENCK

SEQ ID NO: 176 - protein, 13 aa, synthetic construct
GIFVIQSESLKKC

SEQ ID NO: 177 - protein, 10 aa, synthetic construct
WSVLTLENCK

SEQ ID NO: 178 - protein, 31 aa, synthetic construct
WQDKGIFVIQSESLKKCIQAGKSVLTLENCK

SEQ ID NO: 179 - protein, 9 aa, synthetic construct
YDWIPSSAW

SEQ ID NO: 180 - protein, 9 aa, synthetic construct
AGAIWQRDW

SEQ ID NO: 181 - protein, 9 aa, synthetic construct
AGAIWQKDW

SEQ ID NO: 182 - protein, 8 aa, synthetic construct
VIQSESLK

SEQ ID NO: 183 - protein, 8 aa, synthetic construct
PIQSESLK

SEQ ID NO: 184 - protein, 8 aa, synthetic construct
PIESESLK

SEQ ID NO: 185 - protein, 9 aa, synthetic construct
SVLTEENCK

SEQ ID NO: 186 - protein, 8 aa, synthetic construct
  SITE 5: note = Any Naturally-Occurring Amino Acid
  SITE 8: note = Any Naturally-Occurring Amino Acid
SVLTXENX

SEQ ID NO: 187 - protein, 9 aa, synthetic construct
  SITE 5: note = Any Naturally-Occurring Amino Acid
SVLTXENCK

SEQ ID NO: 188 - protein, 8 aa, synthetic construct
  SITE 1: note = Any Naturally-Occurring Amino Acid
  SITE 3: note = Any Naturally-Occurring Amino Acid
  SITE 4: note = Any Naturally-Occurring Amino Acid
  SITE 6: note = Any Naturally-Occurring Amino Acid
XIXXEXLK

SEQ ID NO: 189 - protein, 14 aa, synthetic construct
  MOD_RES 5: note = N-Methylation
  MOD_RES 14: note = N-Methylation
  DISULFID 3..13: note = Cys-Cys
  SITE 1: note = D-Amino Acid
  MOD_RES 9: note = MeGly
YICVWQDWGAHRCI

SEQ ID NO: 190 - protein, 26 aa, synthetic construct
  DISULFID 2..12: note = Cys-Cys
ICVVQDWGHHRCTAGMANLTSHASAI

SEQ ID NO: 191 - protein, 13 aa, synthetic construct
  DISULFID 2..12: note = Cys-Cys
ICVVQDWGHHRCT

SEQ ID NO: 192 - protein, 11 aa, synthetic construct
  DISULFID 1..11: note = Cys-Cys
CVVQDWGHHAC

SEQ ID NO: 193 - protein, 13 aa, synthetic construct
  MOD_RES 1: note = AcIle
  DISULFID 2..12: note = Cys-Cys
  SITE 1: note = N-Terminal Acetyl
  SITE 13: note = C-Terminal Amide
ICVVQDWGHHRCT

SEQ ID NO: 194 - protein, 12 aa, synthetic construct
  DISULFID 1..11: note = Cys-Cys
  SITE 12: note = C-Terminal Amide
CVVQDWGHHRCT

SEQ ID NO: 195 - protein, 11 aa, synthetic construct
  DISULFID 1..11: note = Cys-Cys
  SITE 11: note = C-Terminal Amide
CVVQDWGHHRC

SEQ ID NO: 196 - protein, 13 aa, synthetic construct
  SITE 1: note = N-Terminal Acetyl
  DISULFID 2..12: note = Cys-Cys
  SITE 13: note = C-Terminal Amide
ICVVGDWGHHRCT

SEQ ID NO: 197 - protein, 13 aa, synthetic construct
  SITE 1: note = N-Terminal Acetyl
  DISULFID 2..12: note = Cys-Cys
  SITE 13: note = C-Terminal Amide
ICVVQPWGHHRCT

SEQ ID NO: 198 - protein, 17 aa, synthetic construct
  DISULFID 6..16: note = Cys-Cys
  SITE 1: note = N-Terminal Biotin
  SITE 17: note = C-Terminal Amide
KYSSICVVQDWGHHRCT

SEQ ID NO: 199 - protein, 27 aa, synthetic construct
  SITE 1: note = N-Terminal Acetyl
  DISULFID 2..12: note = Cys-Cys
  SITE 27: note = C-Terminal Biotin
ICVVQDWGHHRCTAGHMANLTSHASAK

SEQ ID NO: 200 - protein, 13 aa, synthetic construct
  SITE 1: note = N-Terminal Acetyl
  MOD_RES 4: note = N-Methylation
ICVWQDWGAHRCT

SEQ ID NO: 201 - protein, 14 aa, synthetic construct
  MOD_RES 9: note = MeGly
  MOD_RES 14: note = N-Methylation
  MOD_RES 5: note = 1-Methylation
YICVWQDWGAHRCI

SEQ ID NO: 202 - protein, 14 aa, synthetic construct
  SITE 1: note = PEG40K
  DISULFID 3..13: note = Cys-Cys
  MOD_RES 5: note = N-Methylation
  MOD_RES 9: note = MeGly
  MOD_RES 14: note = N-Methylation
YICVWQDWGAHRCI

SEQ ID NO: 203 - protein, 15 aa, synthetic construct
  SITE 1: note = N-Terminal Acetyl
  MOD_RES 5: note = N-Methylation
  MOD_RES 9: note = MeGly
  MOD_RES 14: note = N-Methylation
  DISULFID 3..13: note = Cys-Cys
YICVWQDWGAHRCIK

SEQ ID NO: 204 - protein, 15 aa, synthetic construct
  SITE 1: note = N-Terminal Acetyl
  DISULFID 3..13: note = Cys-Cys
  MOD_RES 5: note = N-Methylation
  MOD_RES 9: note = MeGly
  SITE 15: note = C-terminal PEG40K
YICVWQDWGAHRCIK

SEQ ID NO: 205 - protein, 13 aa, synthetic construct
  DISULFID 2..12: note = Cys-Cys
  SITE 1: note = N-Terminal Acetyl
  SITE 13: note = C-Terminal Amide
ICVWQDWGAHRCT

SEQ ID NO: 206 - protein, 13 aa, synthetic construct
  DISULFID 2..12: note = Cys-Cys
  SITE 1: note = N-Terminal Acetyl
  MOD_RES 5: note = N-Methylation
  MOD_RES 8: note = MeGly
  SITE 13: note = C-Terminal Amide
ICVWQDWGAHRCI

SEQ ID NO: 207 - protein, 13 aa, synthetic construct
  SITE 1: note = N-Terminal Acetyl
  MOD_RES 4: note = N-Methylation
  MOD_RES 8: note = MeGly
  MOD_RES 13: note = N-Methylation
  DISULFID 2..12: note = Cys-Cys
ICVWQDWGAHRCI

SEQ ID NO: 208 - protein, 13 aa, synthetic construct
  DISULFID 2..12: note = Cys-Cys
  SITE 1: note = N-Terminal Acetyl
  MOD_RES 4: note = N-Methylation
  SITE 13: note = C-Terminal Amide
ICVWQDWGAHRCT

SEQ ID NO: 209 - protein, 13 aa, synthetic construct
  DISULFID 2..13: note = Cys-Cys
  SITE 1: note = N-Terminal Acetyl
XCVXQDWGXXXCT

SEQ ID NO: 210 - protein, 13 aa, synthetic construct
  DISULFID 2..12: note = Cys-Cys
  SITE 1: note = N-Terminal Acetyl
  SITE 13: note = C-Terminal Amide
ICVVQDWGHHRCT

SEQ ID NO: 211 - protein, 13 aa, synthetic construct
  SITE 1: note = N-Terminal Acetyl
  DISULFID 2..12: note = Cys-Cys
  SITE 13: note = C-Terminal Amide
ICVVQDWGAHRCT

SEQ ID NO: 212 - protein, 13 aa, synthetic construct
  SITE 1: note = N-Terminal Acetyl
  DISULFID 2..12: note = Cys-Cys
  SITE 13: note = C-Terminal Amide
ICVTQDWGHHRCT

SEQ ID NO: 213 - protein, 13 aa, synthetic construct
  SITE 1: note = N-Terminal Acetyl
  DISULFID 2..12: note = Cys-Cys
  SITE 13: note = C-Terminal Amide
ICVSQDWGHHRCT

SEQ ID NO: 214 - protein, 13 aa, synthetic construct
  SITE 1: note = N-Terminal Acetyl
  DISULFID 2..12: note = Cys-Cys
  SITE 13: note = C-Terminal Amide
ICVHQDWGHHRCT

SEQ ID NO: 215 - protein, 13 aa, synthetic construct
  SITE 1: note = N-Terminal Acetyl
  DISULFID 2..12: note = Cys-Cys
  SITE 13: note = C-Terminal Amide
ICVFQDWGHHRCT

SEQ ID NO: 216 - protein, 13 aa, synthetic construct
  SITE 1: note = N-Terminal Acetyl
  DISULFID 2..12: note = Cys-Cys
  SITE 13: note = C-Terminal Amide
ICVYQDWGAHRCT

SEQ ID NO: 217 - protein, 13 aa, synthetic construct
  SITE 1: note = N-Terminal Acetyl
  DISULFID 2..12: note = Cys-Cys
  SITE 13: note = C-Terminal Amide
ICVWQDWGWHRCT

SEQ ID NO: 218 - protein, 13 aa, synthetic construct
  SITE 1: note = N-Terminal Acetyl
  DISULFID 2..12: note = Cys-Cys
  SITE 13: note = C-Terminal Amide
ICVWQDWGHHRCT

SEQ ID NO: 219 - protein, 13 aa, synthetic construct
  SITE 1: note = N-Terminal Acetyl
  DISULFID 2..12: note = Cys-Cys
ICVWQDWGAHRCT

SEQ ID NO: 220 - protein, 13 aa, synthetic construct
  SITE 1: note = N-Terminal Acetyl
  DISULFID 2..12: note = Cys-Cys
  SITE 13: note = C-Terminal Amide
ICVWQDWGAHRCT

SEQ ID NO: 221 - protein, 13 aa, synthetic construct
  SITE 1: note = N-Terminal Acetyl
  DISULFID 2..12: note = Cys-Cys
  SITE 10: note = D-Amino Acid
ICVWQDWGAHRCT

SEQ ID NO: 222 - protein, 13 aa, synthetic construct
  SITE 1: note = N-Terminal Acetyl
  DISULFID 2..12: note = Cys-Cys
  SITE 9: note = D-Amino Acid
ICVWQDWGAHRCT

SEQ ID NO: 223 - protein, 13 aa, synthetic construct
  SITE 1: note = D-Amino Acid, N-Terminal Acetyl
  DISULFID 2..12: note = Cys-Cys
ICVWQDWGAHRCT

SEQ ID NO: 224 - protein, 13 aa, synthetic construct
  DISULFID 2..12: note = Cys-Cys
  SITE 1: note = N-Terminal Acetyl
  SITE 13: note = D-Amino Acid
ICVWQDWGAHRCT

SEQ ID NO: 225 - protein, 13 aa, synthetic construct
  DISULFID 2..12: note = Cys-Cys
  SITE 1: note = N-Terminal Acetyl
  SITE 13: note = C-Terminal Amide
ICVWQDWGAHRCT

SEQ ID NO: 226 - protein, 13 aa, synthetic construct
  DISULFID 2..12: note = Cys-Cys
  SITE 13: note = C-Terminal Amide
WCVWQDWGTNRCW

SEQ ID NO: 227 - protein, 13 aa, synthetic construct
  SITE 1: note = N-Terminal Acetyl
  DISULFID 2..12: note = Cys-Cys
  SITE 13: note = C-Terminal Amide
DCVWQDWGTNKCW

SEQ ID NO: 228 - protein, 13 aa, synthetic construct
  DISULFID 2..12: note = Cys-Cys
  SITE 13: note = C-Terminal Amide
QCVWQDWGQNQCW

SEQ ID NO: 229 - protein, 13 aa, synthetic construct
  DISULFID 2..12: note = Cys-Cys
  SITE 1: note = N-Terminal Acetyl
  SITE 13: note = C-Terminal Amide
ICVWQDWGAHRCW

SEQ ID NO: 230 - protein, 13 aa, synthetic construct
  SITE 1: note = N-Terminal Acetyl
  DISULFID 2..12: note = Cys-Cys
  SITE 13: note = C-Terminal Amide
WCVWQDWGAHRCT

SEQ ID NO: 231 - protein, 13 aa, synthetic construct
  SITE 1: note = N-Terminal Acetyl
  DISULFID 2..12: note = Cys-Cys
  SITE 13: note = C-Terminal Amide
WCVWQDWGAHRCW

SEQ ID NO: 232 - protein, 13 aa, synthetic construct
  SITE 12: note = Homocysteine
  SITE 1: note = N-Terminal Acetyl
  CROSSLNK 2..12: note = Ala-Hcy
  SITE 13: note = C-Terminal Amide
IAVWQDWGAHRXT

SEQ ID NO: 233 - protein, 13 aa, synthetic construct
  SITE 1: note = N-Terminal Acetyl
  DISULFID 2..12: note = Cys-Cys
  MOD_RES 13: note = N-Methylation
  SITE 13: note = C-Terminal Amide
ICVWQDWGAHRCI

SEQ ID NO: 234 - protein, 13 aa, synthetic construct
  SITE 1: note = N-Terminal Acetyl
  DISULFID 2..12: note = Ala-Hcy
  MOD_RES 13: note = N-Methylation
  SITE 12: note = Homocysteine
  SITE 13: note = C-Terminal Amide
IAVWQDWGAHRXI

SEQ ID NO: 235 - protein, 13 aa, synthetic construct
  SITE 1: note = N-Terminal Acetyl
  MOD_RES 4: note = 5f
  DISULFID 2..12: note = Cys-Cys
  SITE 13: note = C-Terminal Amide
ICVWQDWGAHRCT

SEQ ID NO: 236 - protein, 13 aa, synthetic construct
  SITE 1: note = N-Terminal Acetyl
  MOD_RES 4: note = 5-Methylation
  SITE 13: note = C-Terminal Amide
  DISULFID 2..12: note = Cys-Cys
ICVWQDWGAHRCT

SEQ ID NO: 237 - protein, 13 aa, synthetic construct
  SITE 4: note = Naphthylalanine
  SITE 1: note = N-Terminal Acetyl
  DISULFID 2..12: note = Cys-Cys
ICVXQDWGAHRCT

SEQ ID NO: 238 - protein, 13 aa, synthetic construct
  SITE 1: note = N-Terminal Acetyl
  MOD_RES 7: note = 5f
  SITE 13: note = C-Terminal Acetyl
  DISULFID 2..12: note = Cys-Cys
ICVWQDWGAHRCT

SEQ ID NO: 239 - protein, 13 aa, synthetic construct
  SITE 1: note = N-Terminal Acetyl
  MOD_RES 7: note = 5-Methylation
  SITE 13: note = C-Terminal Amide
  DISULFID 2..12: note = Cys-Cys
ICVWQDWGAHRCT

SEQ ID NO: 240 - protein, 13 aa, synthetic construct
  SITE 1: note = N-Terminal Acetyl
  MOD_RES 7: note = 1-Methylation
  SITE 13: note = C-Terminal Amide
  DISULFID 2..12: note = Cys-Cys
ICVWQDWGAHRCT

SEQ ID NO: 241 - protein, 13 aa, synthetic construct
  SITE 1: note = N-Terminal Acetyl
  DISULFID 2..12: note = Cys-Cys
  SITE 13: note = C-Terminal Amide
ICVYQDWGAHRCT

SEQ ID NO: 242 - protein, 13 aa, synthetic construct
  SITE 1: note = N-Terminal Acetyl
  DISULFID 2..12: note = Cys-Cys
ICVWQDWGAHRCT

SEQ ID NO: 243 - protein, 13 aa, synthetic construct
  SITE 1: note = N-Terminal Acetyl
  DISULFID 2..12: note = Cys-Cys
ICVWQDWGAHRCT

SEQ ID NO: 244 - protein, 13 aa, synthetic construct
  SITE 1: note = N-Terminal Acetyl
  SITE 13: note = D-Amino Acid
  DISULFID 2..12: note = Cys-Cys
ICVWQDWGAHRCT

SEQ ID NO: 245 - protein, 13 aa, synthetic construct
  SITE 1: note = N-Terminal Acetyl
  SITE 4: note = Naphthylalanine
  DISULFID 2..12: note = Cys-Cys
ICVXQDWGAHRCT

SEQ ID NO: 246 - protein, 13 aa, synthetic construct
  SITE 1: note = N-Terminal Acetyl
  SITE 4: note = Naphthylalanine
  DISULFID 2..12: note = Cys-Cys
ICVXQDWGAHRCT

SEQ ID NO: 247 - protein, 13 aa, synthetic construct
  SITE 1: note = N-Terminal Acetyl
  SITE 4: note = Naphthylalanine
  DISULFID 2..12: note = Cys-Cys
ICVXQDWGAHRCT

SEQ ID NO: 248 - protein, 13 aa, synthetic construct
  SITE 1: note = N-Terminal Acetyl
  SITE 4: note = Lysoalanine
  DISULFID 2..12: note = Cys-Cys
ICVXQDWGAHRCT

SEQ ID NO: 249 - protein, 13 aa, synthetic construct
  SITE 1: note = N-Terminal Acetyl
  SITE 4: note = Lysoalanine
  DISULFID 2..12: note = Cys-Cys
ICVXQDWGAHRCT

SEQ ID NO: 250 - protein, 13 aa, synthetic construct
  SITE 1: note = N-Terminal Acetyl
  MOD_RES 5: note = htQ
  DISULFID 2..12: note = Cys-Cys
ICVDQDWGAHRCT

SEQ ID NO: 251 - protein, 13 aa, synthetic construct
  SITE 1: note = N-Terminal Acetyl
  SITE 4: note = Boa
  DISULFID 2..12: note = Cys-Cys
ICVXQDWGAHRCT

SEQ ID NO: 252 - protein, 13 aa, synthetic construct
  SITE 1: note = N-Terminal Acetyl
  SITE 4: note = 4-Benzoylphenylalanine
  DISULFID 2..12: note = Cys-Cys
ICVXQDWGAHRCT

SEQ ID NO: 253 - protein, 13 aa, synthetic construct
  SITE 1: note = N-Terminal Acetyl
  SITE 4: note = L-3-Benzothienylalanine
  DISULFID 2..12: note = Cys-Cys
ICVXQDWGAHRCT

SEQ ID NO: 254 - protein, 13 aa, synthetic construct
  SITE 1: note = N-Terminal Acetyl
  SITE 4: note = L-3-Benzothienylalanine
  DISULFID 2..12: note = Cys-Cys
ICVXQDWGAHRCT

SEQ ID NO: 255 - protein, 13 aa, synthetic construct
  SITE 1: note = N-Terminal Acetyl
  SITE 9: note = Abu
  DISULFID 2..12: note = Cys-Cys
ICVWQDWGXHRCT

SEQ ID NO: 256 - protein, 16 aa, synthetic construct
  MOD_RES 1: note = H-Gly
  DISULFID 3..13: note = Cys-Cys
GICVWQDWGAHRCTAN

SEQ ID NO: 257 - protein, 13 aa, synthetic construct
  SITE 1: note = N-Terminal Acetyl
  MOD_RES 4: note = 5f
  DISULFID 2..12: note = Cys-Cys
ICVWQDWGAHRCT

SEQ ID NO: 258 - protein, 13 aa, synthetic construct
  SITE 1: note = N-Terminal Acetyl
  MOD_RES 4: note = 5-Methylation
  DISULFID 2..12: note = Cys-Cys
ICVWQDWGAHRCT

SEQ ID NO: 259 - protein, 13 aa, synthetic construct
  SITE 1: note = N-Terminal Acetyl
  MOD_RES 4: note = 1-Methylation
  DISULFID 2..12: note = Cys-Cys
ICVWQDWGAHRCT

SEQ ID NO: 260 - protein, 13 aa, synthetic construct
  SITE 1: note = N-Terminal Acetyl
  MOD_RES 4: note = 5f
  DISULFID 2..12: note = Cys-Cys
ICVWQDWGAHRCT

SEQ ID NO: 261 - protein, 13 aa, synthetic construct
  SITE 1: note = N-Terminal Acetyl
  MOD_RES 4: note = 5f
  MOD_RES 7: note = 5f
  DISULFID 2..12: note = Cys-Cys
ICVWQDWGAHRCT

SEQ ID NO: 262 - protein, 13 aa, synthetic construct
  SITE 1: note = N-Terminal Acetyl
  MOD_RES 4: note = 5-Methylation
  MOD_RES 7: note = 5f
  DISULFID 2..12: note = Cys-Cys
ICVWQDWGAHRCT

SEQ ID NO: 263 - protein, 13 aa, synthetic construct
  SITE 1: note = N-Terminal Acetyl
  MOD_RES 4: note = 1-Methylation
  MOD_RES 7: note = 5f
  DISULFID 2..12: note = Cys-Cys
ICVWQDWGAHRCT

SEQ ID NO: 264 - protein, 15 aa, synthetic construct
  SITE 1: note = H-Gly, N-Terminal Acetyl
  MOD_RES 4: note = 6f
  MOD_RES 7: note = 6f
  DISULFID 2..12: note = Cys-Cys
GICVWQDWGAHRCTN

SEQ ID NO: 265 - protein, 13 aa, synthetic construct
  SITE 1: note = N-Terminal Acetyl
  MOD_RES 4: note = 1-formyl
  DISULFID 2..12: note = Cys-Cys
ICVWQDWGAHRCT

SEQ ID NO: 266 - protein, 13 aa, synthetic construct
  SITE 1: note = N-Terminal Acetyl
  MOD_RES 4: note = 1-methyoxy
  DISULFID 2..12: note = Cys-Cys
ICVWQDWGAHRCT

SEQ ID NO: 267 - protein, 15 aa, synthetic construct
  MOD_RES 1: note = H-Gly
  MOD_RES 5: note = 5f
  MOD_RES 7: note = 5f
  DISULFID 3..13: note = Cys-Cys
GICVWQDWGAHRCTN

SEQ ID NO: 268 - protein, 13 aa, synthetic construct
  DISULFID 2..12: note = Cys-Cys
  SITE 1: note = N-Terminal Acetyl
  MOD_RES 4: note = Methylation
  SITE 13: note = AEEA
ICVWQDWGAHRCT

SEQ ID NO: 269 - protein, 7 aa, synthetic construct
  SITE 1: note = N-Terminal Acetyl
AEAKAKA

SEQ ID NO: 270 - protein, 15 aa, synthetic construct
IALILEPICCQERAA

SEQ ID NO: 271 - protein, 15 aa, synthetic construct
  SITE 15: note = C-Terminal dPEG24
IALILEPICCQERAA

SEQ ID NO: 272 - protein, 15 aa, synthetic construct
  SITE 1: note = N-Terminal dPEG24
IALILEPICCQERAA

SEQ ID NO: 273 - protein, 15 aa, synthetic construct
RALILEPICCQERAA

SEQ ID NO: 274 - protein, 15 aa, synthetic construct
IRLILEPICCQERAA

SEQ ID NO: 275 - protein, 15 aa, synthetic construct
IARILEPICCQERAA

SEQ ID NO: 276 - protein, 15 aa, synthetic construct
IALIREPICCQERAA

SEQ ID NO: 277 - protein, 15 aa, synthetic construct
IALILEPICCRERAA

SEQ ID NO: 278 - protein, 15 aa, synthetic construct
IALILEPICCQRRAA

SEQ ID NO: 279 - protein, 15 aa, synthetic construct
IELILEPICCQERAA

SEQ ID NO: 280 - protein, 15 aa, synthetic construct
IAEILEPICCQERAA

SEQ ID NO: 281 - protein, 15 aa, synthetic construct
IALILEPICCQEEAA

SEQ ID NO: 282 - protein, 15 aa, synthetic construct
IALILEPICCQEREA

SEQ ID NO: 283 - protein, 15 aa, synthetic construct
IALILEEICCQERAA

SEQ ID NO: 284 - protein, 15 aa, synthetic construct
IALILEPECCQERAA

SEQ ID NO: 285 - protein, 30 aa, synthetic construct
PAICQRATATLGTVGSNTSGTTAIEACILL

SEQ ID NO: 286 - protein, 15 aa, synthetic construct
  DISULFID 1..12: note = Cys-Cys
CEGPFGPRHDLTFCW

SEQ ID NO: 287 - protein, 15 aa, synthetic construct
  SITE 1: note = Xb
  DISULFID 1..12: note = Xb-Cys
XEGPFGPRHDLTFCW

SEQ ID NO: 288 - protein, 7 aa, synthetic construct
QYYPFSX

SEQ ID NO: 289 - protein, 7 aa, synthetic construct
NPFNLAR

SEQ ID NO: 290 - protein, 12 aa, synthetic construct
QLQDMTSSPFWL

SEQ ID NO: 291 - protein, 12 aa, synthetic construct
NPFVIGRWHPPH

SEQ ID NO: 292 - protein, 12 aa, synthetic construct
SLAKFLNPFLYR

SEQ ID NO: 293 - protein, 12 aa, synthetic construct
ASTPRFEPFQLD

SEQ ID NO: 294 - protein, 12 aa, synthetic construct
SLHSQPYSPFML

SEQ ID NO: 295 - protein, 12 aa, synthetic construct
NILSSWSSPFVF

SEQ ID NO: 296 - protein, 12 aa, synthetic construct
NLPSSWTNPFYL

SEQ ID NO: 297 - protein, 7 aa, synthetic construct
SPFMLHP

SEQ ID NO: 298 - protein, 7 aa, synthetic construct
PSPFMLT

SEQ ID NO: 299 - protein, 7 aa, synthetic construct
IGPFHLH

SEQ ID NO: 300 - protein, 7 aa, synthetic construct
TNPFMLN

SEQ ID NO: 301 - protein, 7 aa, synthetic construct
NTTFLYP

SEQ ID NO: 302 - protein, 7 aa, synthetic construct
SHYTQYL

SEQ ID NO: 303 - protein, 7 aa, synthetic construct
NHHPNYW

SEQ ID NO: 304 - protein, 7 aa, synthetic construct
VHYPLSW

SEQ ID NO: 305 - protein, 12 aa, synthetic construct
HHLKYSDTSPPI

SEQ ID NO: 306 - protein, 13 aa, synthetic construct
SHMHERWDTSPPI

SEQ ID NO: 307 - protein, 12 aa, synthetic construct
SHMHERWDTSYQ

SEQ ID NO: 308 - protein, 12 aa, synthetic construct
SHIHSNAAWRIT

SEQ ID NO: 309 - protein, 7 aa, synthetic construct
WHYPHWQ

SEQ ID NO: 310 - protein, 7 aa, synthetic construct
SHYLYTQ

SEQ ID NO: 311 - protein, 7 aa, synthetic construct
AHYSFTQ

SEQ ID NO: 312 - protein, 7 aa, synthetic construct
THYPTFY

SEQ ID NO: 313 - protein, 7 aa, synthetic construct
EHNTSFW

SEQ ID NO: 314 - protein, 7 aa, synthetic construct
NHYKLTW

SEQ ID NO: 315 - protein, 7 aa, synthetic construct
NHSPYFQ

SEQ ID NO: 316 - protein, 7 aa, synthetic construct
SHYQHYQ

SEQ ID NO: 317 - protein, 30 aa, synthetic construct
PAICQRATATLGTVGSNTSGTTEIEACILL

SEQ ID NO: 318 - protein, 10 aa, synthetic construct
WLGLGGGYGW

SEQ ID NO: 319 - protein, 15 aa, synthetic construct
FYGPFFLNDSLRGIW

SEQ ID NO: 320 - protein, 15 aa, synthetic construct
LRFLNPFSLDGSGFW

SEQ ID NO: 321 - protein, 15 aa, synthetic construct
HSPFCLGVLECFGLV

SEQ ID NO: 322 - protein, 15 aa, synthetic construct
TCGAFYLYHDPFICG

SEQ ID NO: 323 - protein, 15 aa, synthetic construct
MQHCLASHELYLPWC

SEQ ID NO: 324 - protein, 15 aa, synthetic construct
FFVFGSGDAFAFSDM

SEQ ID NO: 325 - protein, 15 aa, synthetic construct
PCVIIDTGSSRWCYL

SEQ ID NO: 326 - protein, 15 aa, synthetic construct
HSPFCLGVLECFGLV

SEQ ID NO: 327 - protein, 15 aa, synthetic construct
HAAFEPRGDVRHTLL

SEQ ID NO: 328 - protein, 12 aa, synthetic construct
CRWDGSWGEVRC

SEQ ID NO: 329 - protein, 12 aa, synthetic construct
CYWVGTWGEAVC

SEQ ID NO: 330 - protein, 16 aa, synthetic construct
RWFPCPNKEGCCSISV

SEQ ID NO: 331 - protein, 16 aa, synthetic construct
RSTYCNKNKDSCHIPE

SEQ ID NO: 332 - protein, 16 aa, synthetic construct
QPPQCIKDGGFVICRV

SEQ ID NO: 333 - protein, 16 aa, synthetic construct
KGKKCKPEEHPCNEPM

SEQ ID NO: 334 - protein, 16 aa, synthetic construct
NKMTCSDDGKLCWEHL

SEQ ID NO: 335 - protein, 14 aa, synthetic construct
PLGRPCPTCPLAPS

SEQ ID NO: 336 - protein, 14 aa, synthetic construct
QRMRPCPSCPLAPW

SEQ ID NO: 337 - protein, 14 aa, synthetic construct
WPSRPCPSCPEVPP

SEQ ID NO: 338 - protein, 14 aa, synthetic construct
SCTKDCPTCPLVPV

SEQ ID NO: 339 - protein, 30 aa, synthetic construct
PAIAQRATATLGTVGSNTSGTTEIEACILL

SEQ ID NO: 340 - protein, 30 aa, synthetic construct
PAICQRATATLGTVGSNTSGTTEIEAAILL

SEQ ID NO: 341 - protein, 30 aa, synthetic construct; identical to SEQ ID NO: 285

SEQ ID NO: 342 - protein, 30 aa, synthetic construct
PAICQRATATLGTVGSNTSGTTEIAACILL

SEQ ID NO: 343 - protein, 15 aa, synthetic construct
PAICQRAEIEACILL

SEQ ID NO: 344 - protein, 15 aa, synthetic construct
PAIAQRAEIEAAILL

SEQ ID NO: 345 - protein, 27 aa, synthetic construct
PAICQRATATLGTNTSGTTEIEACILL

SEQ ID NO: 346 - protein, 23 aa, synthetic construct
PAICQRATATLSGTTEIEACILL

SEQ ID NO: 347 - protein, 19 aa, synthetic construct
PAICQRATATTEIEACILL

SEQ ID NO: 348 - protein, 29 aa, synthetic construct
AICQRATATLGTVGSNTSGTTEIEACILL

SEQ ID NO: 349 - protein, 28 aa, synthetic construct
ICQRATATLGTVGSNTSGTTEIEACILL

SEQ ID NO: 350 - protein, 27 aa, synthetic construct
CQRATATLGTVGSNTSGTTEIEACILL

SEQ ID NO: 351 - protein, 29 aa, synthetic construct
PAICQRATATLGTVGSNTSGTTEIEACIL

SEQ ID NO: 352 - protein, 28 aa, synthetic construct
PAICQRATATLGTVGSNTSGTTEIEACI

SEQ ID NO: 353 - protein, 27 aa, synthetic construct
PAICQRATATLGTVGSNTSGTTEIEAC

SEQ ID NO: 354 - protein, 15 aa, synthetic construct
  SITE 1: note = N-Terminal Acetyl
IALILEPICCQERAA

SEQ ID NO: 355 - protein, 30 aa, synthetic construct; identical to SEQ ID NO: 317
  SITE 1: note = N-Terminal Acetyl

SEQ ID NO: 356 - protein, 30 aa, synthetic construct; identical to SEQ ID NO: 339
  SITE 1: note = N-Terminal Acetyl

SEQ ID NO: 357 - protein, 30 aa, synthetic construct; identical to SEQ ID NO: 340
  SITE 1: note = N-Terminal Acetyl

SEQ ID NO: 358 - protein, 30 aa, synthetic construct; identical to SEQ ID NO: 285
  SITE 1: note = N-Terminal Acetyl

SEQ ID NO: 359 - protein, 30 aa, synthetic construct; identical to SEQ ID NO: 342
  SITE 1: note = N-Terminal Acetyl

SEQ ID NO: 360 - protein, 15 aa, synthetic construct
  SITE 1: note = N-Terminal Acetyl
PAICQRAEIEACILL

SEQ ID NO: 361 - protein, 15 aa, synthetic construct
  SITE 1: note = N-Terminal Acetyl
PAIAQRAEIEAAILL

SEQ ID NO: 362 - protein, 27 aa, synthetic construct
  SITE 1: note = N-Terminal Acetyl
PAICQRATATLGTNTSGTTEIEACILL

SEQ ID NO: 363 - protein, 23 aa, synthetic construct
  SITE 1: note = N-Terminal Acetyl
PAICQRATATLSGTTEIEACILL

SEQ ID NO: 364 - protein, 19 aa, synthetic construct
  SITE 1: note = N-Terminal Acetyl
PAICQRATATTEIEACILL

SEQ ID NO: 365 - protein, 28 aa, synthetic construct
  SITE 1: note = N-Terminal Acetyl
ICQRATATLGTVGSNTSGTTEIEACILL

SEQ ID NO: 366 - protein, 27 aa, synthetic construct
  SITE 1: note = N-Terminal Acetyl
CQRATATLGTVGSNTSGTTEIEACILL

SEQ ID NO: 367 - protein, 29 aa, synthetic construct
  SITE 1: note = N-Terminal Acetyl
PAICQRATATLGTVGSNTSGTTEIEACIL

SEQ ID NO: 368 - protein, 28 aa, synthetic construct
  SITE 1: note = N-Terminal Acetyl
PAICQRATATLGTVGSNTSGTTEIEACI

SEQ ID NO: 369 - protein, 27 aa, synthetic construct
  SITE 1: note = N-Terminal Acetyl
PAICQRATATLGTVGSNTSGTTEIEAC

SEQ ID NO: 370 - protein, 15 aa, synthetic construct
IVVTAPADLWDWIRA

SEQ ID NO: 371 - protein, 15 aa, synthetic construct
ITVTMPADLWDWIRA

SEQ ID NO: 372 - protein, 15 aa, synthetic construct
IVVTIPADLWDWIRA

SEQ ID NO: 373 - protein, 15 aa, synthetic construct
IVVTLPADLWDWIRA

SEQ ID NO: 374 - protein, 15 aa, synthetic construct
IVVTVPADLWDWIRA

SEQ ID NO: 375 - protein, 15 aa, synthetic construct
IVVTMPADLWDWIMA

SEQ ID NO: 376 - protein, 15 aa, synthetic construct
IVVTMPADLWDWINA

SEQ ID NO: 377 - protein, 15 aa, synthetic construct
IVVTMPADLWDWIQA

SEQ ID NO: 378 - protein, 15 aa, synthetic construct
IHVTIPADLWDWINK

SEQ ID NO: 379 - protein, 14 aa, synthetic construct
IHVTIPADLWDWIN

SEQ ID NO: 380 - protein, 36 aa, synthetic construct
  SITE 4: note = cyclohexyl-L-alanine
  SITE 14: note = 4-fluoro-L-phenylalanine
  SITE 29: note = gamma-carboxyglutamic acid
QSDXDCIHRLLEAXLDPNLTEEQRWEKIXKINDECE

SEQ ID NO: 381 - protein, 37 aa, synthetic construct
  SITE 4: note = cyclohexyl-L-alanine
  SITE 14: note = 4-fluoro-L-phenylalanine
  SITE 29: note = gamma-carboxyglutamic acid
  SITE 31: note = PEG30L
QSDXDCIHRLLEAXLDPNLTEEQRWERIXKXINDECE

SEQ ID NO: 382 - protein, 37 aa, synthetic construct; identical to SEQ ID NO: 381
  SITE 4: note = cyclohexyl-L-alanine
  SITE 14: note = 4-fluoro-L-phenylalanine
  SITE 29: note = gamma-carboxyglutamic acid
  SITE 31: note = PEG20Br

SEQ ID NO: 383 - protein, 37 aa, synthetic construct; identical to SEQ ID NO: 381
  SITE 4: note = cyclohexyl-L-alanine
  SITE 14: note = 4-fluoro-L-phenylalanine
  SITE 29: note = gamma-carboxyglutamic acid
  SITE 31: note = PEG40Br

SEQ ID NO: 384 - protein, 35 aa, synthetic construct
  SITE 3: note = homoleucine
FDXDCIHRLLEAFLDPNLTEQQRWEKIDKINDECE

SEQ ID NO: 385 - protein, 36 aa, synthetic construct
  SITE 4: note = cyclohexyl-L-alanine
  SITE 14: note = 4-fluoro-L-phenylalanine
  SITE 29: note = gamma-carboxyglutamic acid
QSDXDCIHRLLEAXLDPNLTEEQRWERIXKINDECE

SEQ ID NO: 386 - protein, 38 aa, synthetic construct
  SITE 6: note = cyclohexyl-L-alanine
  SITE 19: note = N-epsilon-acetyl-L-Lys
SWQSDXDCIHRLLEAFLDXNLTEEQRWERIDKINDECE

SEQ ID NO: 387 - protein, 38 aa, synthetic construct
  SITE 6: note = cyclohexyl-L-alanine
  MOD_RES 19: note = 40 kDa branched PEG-Lys
SWQSDXDCIHRLLEAFLDKNLTEEQRWERIDKINDECE

SEQ ID NO: 388 - protein, 29 aa, synthetic construct
  VARIANT 3: note = K, Q, R, S, W and Y
  VARIANT 4: note = selected from A, D, E, F, H, I, K, L, M, N, Q, R, S, T, V and Y
  VARIANT 7: note = selected from F, H, I, K, L, M, N, R, S, T, V, W and Y
  VARIANT 11: note = selected from A, I, K, L, M, N, R, S, T and V
  VARIANT 16: note = selected from N and T
  VARIANT 17: note = selected from A, I, T and V
  VARIANT 20: note = selected from I, L, M, R, T and V
  VARIANT 21: note = selected from A, S, T and V
  VARIANT 25: note = selected from I, M, Q, S, T, V and W
  VARIANT 26: note = selected from K and S
  VARIANT 28: note = selected from F, L, M and Y
  VARIANT 29: note = selected from D and R
  VARIANT 18: note = selected from D, E, G, H, K, N, Q, R, S and T
EEXXAWXEIHXLPNLXXXQXXAFIXXLXX

SEQ ID NO: 389 - protein, 29 aa, synthetic construct
  VARIANT 3: note = selected from A, F, H, K, Q, R, S, W and Y
  VARIANT 4: note = selected from A, D, E, F, H, I, K, L, M, N, Q, R, S, T, V and Y
  VARIANT 7: note = selected from F, H, I, K, L, M, N, R, S, T, V, W and Y
  VARIANT 11: note = selected from A, I, K, L, M, N, R, S, T and V
  VARIANT 16: note = selected from N and T
  VARIANT 17: note = selected from A, I, T and V
  VARIANT 18: note = selected from D, E, G, H, K, N, Q, R, S and T
  VARIANT 20: note = selected from I, L, M, R, T and V
  VARIANT 21: note = selected from A, S, T and V
  VARIANT 25: note = selected from I, M, Q, S, T, V and W
  VARIANT 26: note = selected from K and S
  VARIANT 28: note = selected from F, L, M and Y
  VARIANT 29: note = selected from D and R
EEXXAWXEIHXLPNLXXXQXXAFIXXLXX

SEQ ID NO: 390 - protein, 49 aa, synthetic construct
YYALSDAKEEEPRYKALRGENQDLREKERKYQDKIKKLEEKEKNLEKKS